(19) United States
(12) Patent Application Publication
(10) Pub. No.: US 2022/0283144 A1
(43) Pub. Date: Sep. 8, 2022

(54) COMPOSITIONS AND METHODS IDENTIFYING AND USING STEM CELL DIFFERENTIATION MARKERS

(71) Applicant: The Board of Trustees of the Leland Stanford Junior University, Stanford, CA (US)

(72) Inventors: Lei S. Qi, Stanford, CA (US); Yanxia Liu, Stanford, CA (US)

(73) Assignee: The Board of Trustees of the Leland Stanford Junior University, Stanford, CA (US)

(21) Appl. No.: 17/496,275

(22) Filed: Oct. 7, 2021

Related U.S. Application Data
(63) Continuation of application No. 15/863,005, filed on Jan. 5, 2018, now abandoned.
(60) Provisional application No. 62/443,401, filed on Jan. 6, 2017.

(51) Int. Cl. G01N 33/50; C12N 9/12; C12N 15/65; C12N 9/22; G01N 33/68; C12N 5/071

(52) U.S. Cl. CPC G01N 33/5073; C12N 9/1276; C12N 15/65; C12N 9/22; G01N 33/6887; C12N 5/0697; C12N 2310/20; C12N 2502/1323

(57) ABSTRACT

Provided herein are compositions and methods for identifying and using stem cell regulation factors. For example, in some embodiments, provided herein are compositions and methods for identifying stem cell regulation factors using marker gene expression libraries. Also provided herein are compositions and methods for generating differentiated cells lines and uses of such cell lines.

### CROSS REFERENCE TO RELATED APPLICATIONS

This application is a continuation of U.S. patent application Ser. No. 15/863,005, filed Jan. 5, 2018, which claims the benefit of U.S. Provisional Application No. 62/443,401, filed Jan. 6, 2017, both of which are incorporated herein by reference in their entireties.

### STATEMENT REGARDING FEDERALLY-SPONSORED RESEARCH

The invention was made with Government support under contract OD017887 awarded by the National Institutes of Health. The Government has certain rights in the invention.

### SEQUENCE LISTING

The instant application contains a Sequence Listing which has been filed electronically in ASCII format and is hereby incorporated by reference in its entirety. Said ASCII copy, created Oct. 6, 2021, is named 079445-1273450-006220US_SL.txt and is 1.47 MB (1,547,157) bytes in size.

### FIELD

Provided herein are compositions and methods for identifying and using stem cell differentiation regulation factors. For example, in some embodiments, provided herein are compositions and methods for identifying stem cell differentiation regulation factors using marker gene expression libraries. Also provided herein are compositions and methods for generating differentiated and induced cells lines and uses of such cell lines.

### BACKGROUND

Stem cells are cells that are capable of differentiating into many cell types. Embryonic stem cells are derived from embryos and are potentially capable of differentiation into all of the differentiated cell types of a mature body. Certain types of stem cells are “pluripotent,” which refers to their capability of differentiating into many cell types. One type of pluripotent stem cell is the human embryonic stem cell (hESC), which is derived from a human embryonic source. Human embryonic stem cells are capable of indefinite proliferation in culture, and therefore, are an invaluable resource for supplying cells and tissues to repair failing or defective human tissues in vivo.

Similarly, induced pluripotent stem (iPS) cells, which may be derived from non-embryonic sources, can proliferate without limit and differentiate into each of the three embryonic germ layers. It is understood that iPS cells behave in culture essentially the same as ESCs. Human iPS cells and ES cells express one or more pluripotent cell-specific markers, such as Oct-4, SSEA-3, SSEA-4, Tra 1-60, Tra 1-81, and Nanog (Yu et al. Science, Vol. 318. No. 5858, pp. 1917-1920 (2007); herein incorporated by reference in its entirety). Also, recent findings of Chan, indicate that expression of Tra 1-60, DNMT3B, and REX1 can be used to positively identify fully reprogrammed human iPS cells, whereas alkaline phosphatase, SSEA-4, GDF3, hTERT, and NANOG are insufficient as markers of fully reprogrammed human iPS cells. (Chan et al., Nat. Biotech. 27:1033-1037 (2009); herein incorporated by reference in its entirety).

The cell fate decision making of stem cells is governed by multistep dynamic processes, in which transcriptional networks play a critical role (Chambers and Tomlinson, 2009 Development 136, 2311-2322; Filipczyk et al., 2015 Nat. Cell Biol. 17, 1235-1246; Kim et al., 2008 Cell 132, 1049-1061; MacArthur et al., 2009 Nat. Rev. Mol. Cell Biol. 10, 672-681). Expression of different transcription factors coordinate to activate or suppress sets of genes specific to different lineages, serving as major regulators that maintain cell identities or drive cell fate transitions (Iwafuchi-Doi and Zaret, 2014 Genes Dev. 28, 2679-2692; Zaret and Carroll, 2011 Genes Dev. 25, 2227-2241). The successes of somatic cell reprogramming and directed lineage differentiation using transcription factors highlight their central role in cell fate determination (Davis et al., 1987 Cell 51, 987-1000; Takahashi and Yamanaka, 2006 Cell 126, 663-676; Vierbuchen et al., 2010 Nature 463, 1035-1041; Xu et al., 2015 Cell Stem Cell 16, 119-134). Over the past few decades, although individual or combinatorial transcription factors have been identified for cell differentiation, there is a dearth of systematically unbiased studies of how specific genetic programs determine cell fate maintenance and transitions. Because of this, the available tools to control stem cell differentiation are limited and the full promise of stem cells as therapeutic, drug screening, and research tools have gone unmet.

A systematic screening approach to profile and characterize all transcription factors is needed to offer new insights into their contributions to cell fate decisions, which greatly enhances the ability to manipulate cell fate for both basic research and therapeutic purposes.

### SUMMARY

Provided herein are compositions and methods for identifying and using stem cell differentiation regulation factors. For example, in some embodiments, provided herein are compositions and methods for identifying stem cell differentiation regulation factors using marker gene expression libraries. Also provided herein are compositions and methods for generating differentiated and induced cells lines and uses of such cell lines.

The compositions, systems, kits, and methods of the present disclosure overcome limitations of existing technologies to identify transcription factors and nucleic that drive differentiation of pluripotent cells. The transcription factors identified using the described methods find use in research, screening, and therapeutic applications.

In some embodiments, provided herein are systems and methods for identifying factors involved in (e.g., that regulate or control) the differentiation of stem cells by employing a CRISPR activation (CRISPRa)-mediated gain-of-function screening platform. In some such embodiments, a reporter stem cell line is generated that comprises components of a CRSIPR activation system. In some embodiments, the cell line is exposed to an sgRNA library targeting all putative transcription factors or other candidate factors that may be involved in a cellular differentiation process.

In some embodiments, the CRISPR activation system comprises a dCas9 construct under the transcriptional control of a first promoter. In some embodiments, the dCas9 is fused to a peptide epitope. In some embodiments, the activation system further comprises a VP64 transactivation domain under the transcriptional control of a second promoter. In some embodiments, the VP64 transactivation domain is fused to a peptide that specifically binds to the peptide epitope. In some embodiments, the activation system further comprises a selection marker under the transcriptional control of a third promoter. In some embodiments, each of the first, second, and third promoters are different than each other.

For example, in some embodiments, provided herein is a method of identifying pluripotent cell differentiation markers, comprising: a) generating a pluripotent cell line that expresses i) nuclease dead Cas9 fused to a plurality of peptide epitopes; ii) a single chain variable chain antibody fragment specific for the peptide epitope fused to a VP64 tranactivator domain; and iii) a transactivator polypeptide; b) contacting the cell line with a plurality of single guide RNAs (sgRNAs) specific for activation of pluripotent cell differentiation factors to generate a gene activation library; c) sorting the library to identify pluripotent cells that retain pluripotency or differentiate; and d) identifying cell differentiation factors that induce or prevent differentiation of the pluripotent cells. In some embodiments, the differentiation factors are transcription factors or non-coding (e.g., lincRNAs). In some embodiments, the cells are further contacted with a plurality of non-targeting sgRNAs (e.g., to serve as a negative control). In some embodiments, the cells further overexpress endogenous POU domain, class 3, transcription factor 2 (Brn2). In some embodiments, each cell differentiation factors is targeted with a plurality (e.g., at least 10, 20, 30, 40, 50, 60, 70, 80, 90, or 100) distinct sgRNAs. In some embodiments, the cells that retain pluripotency are identified by screening for expression of SSEA1 after culture in media lacking inhibitors of GSK3 and ERK pathways. In some embodiments, cells that differentiate are identified by expression of a differentiation marker. For example, in some embodiments, cells that differentiate into neuronal cells express Tuj1. In some embodiments, the identifying comprises sequencing of sgRNAs after selection for cells that retain pluripotency or differentiate. In some embodiments, the sequencing further comprises comparing the level of the sgRNAs to the level of non-targeting sgRNAs. In some embodiments, cell differentiation factors that retain pluripotency are one or more of the regulation factors shown in FIG. 3 or Table 3. In some embodiments, cell differentiation factors that are associated with differentiation into neuronal cells are one or more of the regulation factors shown in FIG. 6 or Tables 4 and 10. In some embodiments, the sgRNAs are dual-sgRNA-constructs comprising two sgRNAs. In some embodiments, the method further comprises contacting the cell differentiation factors with a fibroblast cell line and identifying cell differentiation factors that promote transdifferentiation of the fibroblast cell line. In some embodiments, the fibroblast cell line is contacted with combinations of two or more cell differentiation factors. In some embodiments, the cell differentiation factors that promote transdifferentiation are a combination of Ezh2 or Ngn1 and one or more additional markers (e.g., Ngn1+Brn2, Brn2+Ezh2, Mecom+Ezh2, Ngn1+Ezh2 or Ngn1+Foxo1).

In some embodiments, the pluripotent cells are induced pluripotent stem cells, adult stem cells, or embryonic stem cells. In some embodiments, the method further comprises the step of activating pairs or groups of pluripotent cell differentiation factors.

In some embodiments, the method comprises or further comprises the step of performing a CRISPR gene repression screen. For example, in some embodiments, the CRISP repression screen comprises: a) contacting a pluripotent cell that expresses dCas9 fused to a transcription repressor domain with a plurality of sgRNAs specific for repression of a plurality of cell differentiation factors; b) sorting the library to identify cells that retain pluripotency or differentiate; and c) identifying cell differentiation factors that induce or prevent differentiation of said pluripotent cells. In some embodiments, the CRISPR repression screen and the CRISPR activation screen are performed in the same or different pluripotent cells. In some embodiments, the CRISPR repression screen and the CRISPR activation screen are performed simultaneously using vectors comprising a first sgRNA specific for activation of a first cell differentiation factor and a second sgRNA specific for repression of a second cell differentiation factor.

Further embodiments provide a library of pluripotent cells generated by the methods descried herein.

Additional embodiments provide a kit or system, comprising: a) a pluripotent cell line that expresses i) nuclease dead Cas9 fused to a plurality of peptide epitopes; ii) a single chain variable chain antibody fragment specific for the peptide epitope fused to a VP64 tranactivator domain; and iii) a transactivator polypeptide; and b) a plurality of single guide RN As (sgRNAs) specific for activation of pluripotent cell differentiation factors. In some embodiments, the kit or system further comprises reagents for analysis of one or more properties (e.g., pluripotency or differentiation) of the cell lines. In some embodiments, the kit or system further comprises reagents for sequencing the cells to identify the presence of said sgRNAs. In some embodiments, the system comprises or further comprises a CRISPR repression system as described herein. In some embodiments, the system comprises one or more sgRNAs (e.g., 10 or more, 100 or more, 1000 or more, or 5000 or more) described in Table 13 (e.g., SEQ ID NOs:586-8317).

Yet other embodiments provide a method of determining the differentiation status of pluripotent or somatic cells, comprising: a) assaying the cells for the expression of one or more transcription factors or lincRNAs selected from those in FIGS. 3 and 6 and Tables 3 and 4; and b) determining the differentiation status of the cells based on the expression. In some embodiments, the presence or increased level of the cell transcription factors in FIG. 3 or Table 3 are indicative of cells that retain pluripotency. In some embodiments, the cell transcription factors are not Nanog, Sox2, Klf4, or Oct4. In some embodiments, the cell transcription factors selected from, for example, Mixip, Etv2, Zc3h11a, Zfp36, Isl2, Tfeb, Fig1a, Hsf2, or Hoxc11 are indicative of cells that retain pluiripotency. In some embodiments, the presence or increased level of the cell transcription factors shown in FIG. 6 or Table 4 is indicative of cells that have differentiated into neuronal cells. In some embodiments, the cell differentiation factors are not Neurog1, Brn2, or KIlf12. In some embodiments, the cell differentiation factors are selected from, for example, Ezh2, Suz12, or Jun.

Still further embodiments provide a method of differentiating pluripotent or somatic (e.g., fibroblast) cells into neuronal cells, comprising: inducing expression of one or more cell regulation factors shown in FIG. 6 or Table 4 in the pluripotent cells. In some embodiments, the cell differentiation factors are selected from, for example, Ezb2, Ngn1, Suz12, or Jun. In some embodiments, the inducing expression comprises contacting the pluripotent cells with a nucleic acid encoding one or more of the cell differentiation factors, contacting the pluripotent cells with an sgRNA that induces expression of one or more of the cell differentiation factors, or contacting the pluripotent cells with a small molecule that induces expression of the cell differentiation factors. In some embodiments, the method further comprises the step of determining the presence of increased level of expression of the cell differentiation factors shown in FIG. 6 or Table 4. In some embodiments, the presence or increased level of the cell differentiation factors shown in FIG. 6 or Table 4 is indicative of cells that have differentiated into neuronal cells.

Certain embodiments provide differentiated cells generated by the methods described herein.

Embodiments of the present disclosure provide a plurality of neuronal cells that express one or more cell differentiation regulation factors shown in FIG. 6 or Table 4 (e.g., one or more of Ezh2, Suz12 or Jun).

Further embodiments provide a method of inducing pluripotency or maintaining pluripotency of a cell line (e.g., a somatic or pluripotent cell line), comprising: inducing expression of one or more cell regulation factors shown in FIG. 3 or Table 3 in said cells (e.g., one or more of Mlxip, Etv2, Zinc Zc3h11a, Zfp36, Isl2, Tfeb, Fig1a, Hsf2, or Hoxc11).

Still other embodiments provide a plurality of pluripotent cells generated or maintained by the methods described herein.

In other embodiments, the present disclosure provides a plurality of pluripotent or iPSCs cells that express one or more cell regulation factors shown in FIG. 3 or Table 3 (e.g., one or more of Mlxip, Etv2, Zinc Zc3h11a, Zfp36, is12, Tfeb, Fig1a, Hsf2, or Hoxc11).

Some embodiments provide a method of transplanting cells, comprising: transplanting differentiated cells generated by the methods described herein into a subject in need thereof (e.g., a subject diagnosed with a disease or condition).

Further embodiments are described herein.

### BRIEF DESCRIPTION OF THE DRAWINGS

FIG. 1A-D shows that enhanced CRISPR activation mouse ES (CamES) cells allow efficient single sgRNA-directed gene activation and stem cell fate control. (A) Engineered eCRISPRa system in mouse ES cells for single sgRNA-mediated self-renewal and differentiation control. (B) A panel of sgRNAs tiling along the upstream regulatory region of Asc11 relative to transcription start site (TSS) in CamES cells show a gradient of efficient gene activation. (C) Effective neural (day 8) and muscle (day 12) differentiation of CamES cells using a single sgRNA to activate endogenous genes. (D) Time-course measurement of endogenous gene expression (Asc11, Brn2, Tuj1, and Map2) during differentiation for CamES cells −sgRNA, +negative control sgRNA, or +sgAsc11, and E14 mouse ES cells +Asc11 cDNA.

FIG. 2A-E shows the use of an sgRNA library to screen genes that maintain pluripotency and self-renewal in mouse ES cells. (A) Schematic representation of CRISPRa-mediated gain-of-function screening (dropout) of genes that maintain pluripotency and self-renewal in CamES cells using a sgRNA library. (B) Flow cytometry data of library-transduced CamES cells during serial passages and after SSEA1 sorting. Negative control, isotype antibody control. (C) Microscopic images showing bright Feld (BF), Oct4 staining, and DAPI of library-transduced CamES cells in −2i medium at passage 2, passage 10 before SSEA1 sorting and passage 10 after sorting. Scale bar, 100 μm. (D) Boxplot of normalized sgRNA counts for the plasmid library, library-transduced CamES cells at D0, and library-transduced CamES cells after SSEA1 sorting. (E) Detected sgRNA counts (sgRNAs with at least one count) in the plasmid library, library-transduced CamES cells at D0, and library-transduced CamES cells after SSEA1 sorting.

FIG. 3A-C shows validation of top hits from the CRISPRa self-renewal screen. (A) A scatter plot showing enrichment of sgRNAs for ranked top hit genes. (B) Fold change of mRNA expression measure by quantitative PCR for each gene using their individual sgRNA in CamES cells. (C) Microscopic images and flow cytometry analysis of pluripotency markers Oct4, Nanog, and SSEA1 in CamES cells transduced with 18 individual sgRNAs in −2i medium after 10 passages.

FIG. 4A-D shows functional characterization and deep sequencing analysis of sgMlxip-transduced CamES cells confirm maintenance of pluripotency in −2i medium. (A) Spontaneous differentiation of sgMlxip- or sgKlf2-transduced CamES cells after 10 passages shows generation of three germ layers. (B) RNA-seq paired scatter plot analysis of the Wnt pathway gene expression comparing CamES +sgMlxip in −2i medium with CamES +2i medium (left, R²=0.81), and comparing CamES −sgMlxip in −2i medium and CamES −2i medium at day 7 (right, R²=0.35). (C) RNA-seq scatter plot analysis of the MAPK pathway gene expression comparing CamES +sgMlxip in −2i medium with CamES +2i medium (left, R²=0.90), and comparing CamES +sgMlxip in −2i medium and CamES −2i medium at day 7 (right, R²=0.59). (D) Normalized mRNA expression for genes in the PI3K pathway for CamES +sgMlxip in −2i medium, CamES +2i medium, and CamES −2i medium at day 7.

FIG. 5A-D shows the use of sgRNA library to screen genes that promote neural differentiation of mouse ES cells. (A) Schematic representation of CRISPRa-mediated gain-of-function screening (non-dropout) of genes that promote neural differentiation in CamES cells using an sgRNA library. (B) Quantification by qPCR for neural marker Tuj1 and Map2 expression before and after MACS sorting. (C) Boxplot of normalized sgRNA counts for the plasmid library, sorted Tuj1-hCD8+ cells, and sorted Tuj1-hCD8− cells. (D) Detected sgRNA counts (sgRNAs with at least one count) in the plasmid library, sorted Tuj1-hCD8+ cells, and sorted Tuj1-hCD8− cells.

FIG. 6A-F shows validation of top hits from CRISPRa neural differentiation screen. (A) Scatter plot of sgRNA enrichment for ranked top hit genes. Only sgRNAs enriched in both replicates are shown. 20 genes and their most enriched sgRNAs (orange) are chosen for validation. (B) Fold change of mRNA expression measure by quantitative PCR for each gene using their individual sgRNA in Tuj1-hCD8 CamES cells. (C) Quantification of hCD8+ cells measured by flow cytometry in Tuj1-hCD8 CamES cells transduced without sgRNA, with 6 individual non-targeting sgRNAs, and with 20 individual sgRNA hits after 12-day differentiation. (D) Quantification of NCAM+ cells measured by flow cytometry in CamES cells transduced without sgRNA, with 6 individual non-targeting sgRNAs, and with 20 individual sgRNA hits after 12-day differentiation. (E) Microscopic images ofMap2 staining in Tuj1-hCD8 CamES cells transduced with individual sgRNAs after 12-day differentiation. Scale bar, 100 μm. (F) Characterization of staining various neural lineage markers (Tuj1, Map2, NeuN, Olig2, GFAP, and vGluT1) in Tuj1-hCD8 CamES cells transduced with individual sgRNAs after 12-day differentiation.

FIG. 7A-G shows functional characterization and deep sequencing analysis of sgJun-mediated CamES neural differentiation. (A) Representative traces of membrane potentials of differentiated neurons from Tuj1-hCD8 CamES cells transduced with sgJun in response to step-voltage (left) and step-current injections (right). (B) Principle component analysis of RNA-seq samples from D0, D2, D5, and D12 of sgJun-transduced CamES cells. (C) RNA-seq analysis showing time-course expression of 6 pluripotency genes and 6 neural lineage genes during differentiation of sgJun-transduced CamES cells. Error bars, s.d.±the mean of four biological replicates. (D) Gene ontology analysis of genes that are enriched in D5 and D12 differentiated neural cells (left, D5 versus D0; right, D12 versus D0). (E) Western blot showing protein expression of Jun and phosphorylated Jun at different time points during differentiation. P-Jun: phosphorylated Jun. (F) RNA-seq paired scatter plot analysis of the downstream genes targeted by the AP-1 complex formed between Jun and c-Fos. Left, D2 versus D0 (p=0.2); middle, D5 versus D0 (p=0.002); and right, D12 versus D0 (p=0.004). (G) Gaussian kernel density plot of expression of the Wnt pathway genes in sgJun-directed differentiated cells at different time points during the neural differentiation.

FIG. 8A-H shows generation of eCRISPRa by systematic optimization of the CRISPRa-SunTag system. (A) A multiple lentiviral eCRISPRa system. (B) Comparison of endogenous Brn2 activation efficiency using 12 individual sgRNAs targeting Brn2 or their mixture for the SFFV-driven scFv-stGFP-VP64 CRISPRa system. (C) Comparison of endogenous Brn2 activation efficiency for different promoters driving scFv-sfGFP-VP64. Data is normalized to the −sgRNA sample. (D) Comparison of endogenous Brn2 activation efficiency for 6 clonal cell lines each generated from EF1a- or PGK-driven scFv-sfGFP-VP64 systems. Data is normalized to the −sgRNA sample. (E) Comparison of endogenous Brn2 activation efficiency for 28 clonal cell lines generated from the PGK-driven scFv-sfGFP-VP64 system. (F) Characterization of CamES cells for the morphology, expression of pluripotency marker Oct4, and expression of eCRISPRa components. (G) Negative staining of Tuj1 (red) in CamES cells, CamES cells +sgControl, and E14 mouse ES cells +sgAsc11 after 12-day differentiation. DAPI is shown in blue. (H) Microscopic images showing cell morphology of CamES cells +sgAsc11 (top) and E14 mouse ES cells +Asc11 cDNA (bottom) at D0, D6, and D12 during differentiation.

FIG. 9A-B shows an experimental procedure and characterization of the CRISPRa self-renewal screen in mouse ES cells. (A) Time line scheme of the gain-of-function self-renewal screen using the sgRNA library. (B) Correlation of sequenced sgRNA counts in library-transduced CamES cells at D0 and after SSEA1 sorting.

FIG. 10 shows a ranked gene list based on the dropout self-renewal screen described in FIGS. 2 and 3.

FIG. 11A-C shows RNA sequencing and characterization of CamES cells +sgMlxip or +sgKlf2 cultured in −2i medium. (A) Heatmap illustrating mRNA expression of the pluripotency-associated genes and lineage specific genes for indicated samples. (B) Histogram plot showing distribution of ratios of the Wnt pathway gene expression for indicated samples. (C) mRNA expression of indicated MAPK pathway genes in CamES cells in −2i medium at D7, in +2i medium, and transduced with sgMlxip in −2i medium.

FIG. 12A-F shows an experimental procedure and characterization of CRISPRa gain-of-function neural differentiation screen. (A) Sequencing results of the Tuj1 locus in Tuj1-hCD8 CamES cells. (B) Flow cytometry data (right) showing the hCD8+ percentage of cells in sgAsc11-transduced Tuj1-hCD8 CamES cells after 8-day differentiation. (C) Comparison of Tuj1 and Map2 mRNA expression levels in differentiated cells with various initial seeding cell densities. (D) Quantification of Tuj1 and Map2 mRNA expression levels in CamES cells, CamES cells +sgControl, and CamES cells +sgLibrary during differentiation. (E) Staining of neural markers Tuj1 and Map2 in library-transduced Tuj1-hCD8 CamES cells. (F) Time line scheme of the gain-of-function neural differentiation screen using the sgRNA library in Tuj1-hCD8 CamES cells.

FIG. 13 shows a ranked gene list based on non-dropout neural differentiation screen shown in FIGS. 5 and 6.

FIG. 14A-F shows characterization of sgJun-directed neural differentiated cells and analysis of dropout and non-dropout screens. (A) Heatmap illustrating mRNA expression of representative pluripotency-associated, progenitor neural lineage, terminal neural lineage, endoderm lineage, and mesoderm lineage genes. (B) Time-course of normalized RNA-seq mRNA counts of 12 genes in the MAPK and Wnt pathways during sgJun-direct CamES cells differentiation. (C) A hypothesized model for endogenous Jun activation-induced neural differentiation by sgJun. (D) Toy example of dropout (left) and non-dropout screens (right). In dropout screens, negative cells drop out of the population and have little noticeable effect. (E) The percentage of screen hits in common with Tuj1-hCD8+/D0 for the Tuj1-hCD8−/D0. SSEA1+/D0, and Tuj1-hCD8+/Tuj1-hCD8− gene ranks at a given hit cutoff. (F) The top ten enriched genes as calculated for Tuj1-hCD8+ relative to day 0, Tuj1-hCD8− relative to day 0, and Tuj1-hCD8+ relative to Tuj1-hCD8-.

FIG. 15A-G shows a CRISPRi experimental screening platform for studying genetic interactions. (A) The experimental setup of the single and double CRISPRi screening platform for GI studies. (B-E) Characterization of biological replicates for single and double sgRNA libraries (R1—biological replicate 1; R2, biological replicate 2): (B) single library without Dox at day 20; (C) single library with Dox at day 20; (D) double library without Dox at day 16; (E) double library with Dox at day 16. (F) Comparison of single library with and without Dox at day 20. (G) Comparison of double library with and without Dox at day 16.

FIG. 16A-F shows a time-course comparison of sgRNA enrichment for single and double libraries and validation of sgRNA pairs for epistatic interactions. (A) Comparing day 0 sample to other time points (grey—day 3; red—day 7; blue—day 13) in the presence of Dox for the single library. (B) The 20 genes among 112 epigenetic factor genes that showed consistent depletion over time due to CRISPRi inhibition. (C) Comparing day 0 sample to other time points (grey—day 8; blue—day 16) in the presence of Dox for the double library. For the comparison without Dox, refer to Fig. S4B. (D) A selected combinations that showed consistent depletion over time due to multiplexed CRISPRi inhibition. (E-F) Validation of two pairwise sgRNAs (MRGBP & MED6; BRD7 & LEO1) for their combinatorial effects in suppressing cell growth and endogenous gene expression.

FIG. 17 shows a module map of chromatin-related genes based on a curated set of protein complexes.

FIG. 18A-E shows (A) Schematic representation of CRISPRa-mediated gain-of-function screenings that promote neuronal differentiation in CamES cells using an sgRNA library. (B) Frequency histograms of the top 3 enriched sgRNAs targeting genes indicated. (C) Quantification of PSA-NCAM+ cells were measured by flow cytometry in CamES cells transduced with three individual sgRNAs of each gene after 12-day differentiation. (D) Microscopic images of Map2 staining in CamES cells transduced with individual sgRNAs after 12-day differentiation. Scale bar, 100 μm. (E) Staining of various neuronal lineage markers (NeuN, Olig2, GFAP, GABA, and vGluT1) in CamES cells transduced with individual sgRNAs after 12-day differentiation.

FIG. 19A-F shows (A) Schematic representation of CRISPRa-mediated gain-of-function double screenings that promote neuronal differentiation in CamES cells using a double sgRNA library. (B) Schematic of the two-guide vector. (C) Reproducibility between the two replicates of the paired CRISPR screen of gene-targeting and negative control (or vice versa) guide pairs, mean±s.e.m. (standard error of the mean). (D) Interaction scores for a pair were computed by subtracting off the maximum of the guide-level effect sizes. (E) Interaction forming capacities of the two sgRNAs inducing different gene activation levels. (F) Quantification of PSA-NCAM+ cells measured by flow cytometry in CamES cells transduced with one single sgRNA or double sgRNAs after 12-day differentiation. Error bars represent standard deviation of three independent experiments.

FIG. 20A-E shows (A) Quantification of MAP2+ cells from MEFs infected with different gene combinations. Averages from 20 randomly selected visual fields are shown. Error bars indicate±s.d. (B) Representative images of Tuj1 staining of MEFs infected with different genes or gene combinations. Scale bar, 100 μm. (C) Ngn1 and Ezh2 induced MEF neuron cells express MAP2, Tuj1 and NeuN, synapsin, and GABA 14 days after infection. Scale bar, 100 μm. (D) Bar graph showing the percentage of Tbr1-positive neurons (Tbr1+) and GABA-positive neurons (GABA+) out of total neurons. (E) Ngn1 and Ezh2 induced perinatal TTF neuron cells express MAP2, Tuj1 and NeuN, synapsin, and GABA 26 days after infection. Scale bar, 100 μm.

FIG. 21A-I shows that MEF-derived induced neurons show functional synaptic properties. (A) Recording electrode patched onto a sfGFP-positive cell with a stimulation electrode (middle panel). The right panel is a merged picture of BF and fluorescence images showing that the recorded cell is sfGFP-positive. (B) Representative traces of whole-cell currents in voltage-clamp mode; cells were held at −80 mV. Step depolarization from 70 mV to +40 mV at 10-mV intervals was delivered (lower panel). (C) Representative trace of evoked membrane potential by +40 pA current injection (lower panel) in current-clamp mode held at −75 mV. Application of 100 nM tetrodotoxin (TTX), a selective blocker of voltage-gated sodium channels, inhibited the action potential. (D) Inward sodium currents were evoked from an induced neurons, and application of 500 nM TTX inhibited these currents. Step depolarization from −70 mV to +60 mV at 10-mV intervals was delivered; cells were held at −80 mV (right panel); a presentative trace of whole-cell current with and without TTX at −10 mV membrane potential in voltage-clamp mode is shown (left panel). (E) Outward potassium currents were evoked from an induced neurons, and application of 5 mM tetraethylammonium (TEA) inhibited these currents. Step depolarization from −70 mV to +60 mV at 10-mV intervals was delivered; cells were held at −80 mV (right panel); a presentative trace of whole-cell current with and without TEA at +60 mV membrane potential in voltage-clamp mode is shown (left panel). (F) Spontaneous EPSCs were recorded from induced neurons. (G) Spontaneous action potentials recorded from an induced neuron (left panel). Application of 100 nM TTX blocked the action potentials (middle panel). Washout of TTX reversed the blockade (right panel). (1-) Representative traces of evoked excitatory spontaneous postsynaptic currents (EPSCs) recorded from an induced neuron (left panel). Application of 30 μM DNQX (6,7-dinitroquinoxaline-2,3-dione), an AMPA/kainate glutamate receptor antagonist, blocked the response of EPSCs (middle panel). Washout of DNQX reversed the blockade (right panel). (1) Representative traces of evoked EPSCs recorded from an induced neuron (left panel). Application of 30 μM BIC (Bicuculline), a GABA receptor antagonist, slightly increased the frequency and amplitude of EPSCs (middle panel). Washout of BIC reversed the increase (right panel). F, and H-I, Cells were recorded at a holding potential (Vh) of −60 mV. Error bars indicate±s.d. of cell counts. Scale bar, 10 μm.

FIG. 22A-E shows generation of the CRISPRa and CRISPRa knock-in cell lines. (A) A multiple lentiviral CRISPRa system. (B) Characterization of CamES cells for the morphology, expression of pluripotency marker Oct4, and expression of CRISPRa components. Scale bars, 100 μm. (C) Schematic of the clonal CamES cell line carrying a biallelic IRES-hCD8 insertion at the Tuj1 locus. (D) Sequencing results of the Tuj1 locus in Tuj1-hCD8 CamES cells. (E) Quantification by qPCR for neuronal markers Tuj1 and Map2 expression before and after MACS sorting.

FIG. 23A-G shows (A) Time line scheme of the neural differentiation screens using the sgRNA library in Tuj1-hCDS CamES cells. (B) Quantification of Tuj1 and Map2 mRNA expression levels in CamES cells, CamES cells +sgControl, and CamES cells +sgLibrary during differentiation. Error bars, s.d.±the mean of three independent experiments. (C) Staining of neural markers Tuj1 and Map2 in library-transduced Tuj1-hCD8 CamES cells. Scale bar, 100 μm. (D) Boxplot. of normalized sgRNA counts for the plasmid library, sorted Tuj1-hCD8+ cells, and sorted Tuj1-hCD8− cells. (E) The top ten enriched genes as calculated for Tuj1-hCD8+ relative to day 0, Tuj1-hCD8− relative to day 0, and Tuj1-hCD8+ relative to Tuj1-hCD8−. (F) Toy example of sgRNA stochastic representation in the screening system. (G) The percentage of screen hits in common with Tuj1-hCDS+/D0 for the Tuj1-hCD8−/D0, SSEA1+/D0, and Tuj1-hCD8+/Tuj1-hCD8− gene ranks at a given hit cutoff.

FIG. 24A-D shows (A) Quantification of PSA-NCAM+ cells were measured by flow cytometry in CamES cells transduced with three individual sgRNAs of each gene after 12-day differentiation. (B) Quantification of hCD8+ cells measured by flow cytometry in Tuj1-hCD8 CamES cells transduced without sgRNA, with 6 individual non-targeting sgRNAs, and with 19 individual sgRNAs after 12-day differentiation. Error bars represent standard deviation of three independent experiments. (C) Quantification of PSA-NCAM+ cells were measured at day 10 by flow cytometry in E14 cells after induction of different transgenes or negative control transgene BFP. Error bars represent standard deviation of three independent experiments. (D) Staining of various neuronal lineage markers (Tuj1, NeuN, Olig2, GFAP, GABA, and vGluT1) in CamES cells transduced with individual sgRNAs after 12-day differentiation. Scale bar, 100 μm.

FIG. 25 shows quantification of MAP2+ cells from MEFs infected with different genes.

FIG. 26A-E shows (A) The distribution of guides for the top 19 hits in green against an equal number of randomly selected negative control guides. (B) Variable gene effects and mixing proportions. (C) The estimated gene effect sizes plotted versus the estimated gene specific mixing proportions. (D) The estimated feature coefficients and their 80% credible interval from the model described in Example 4. (E) The distribution of average log 2 fold change of guides in the corresponding feature (top).

FIG. 27A-D shows (A) Cloning strategy for final two-guides vector. (B) Sequencing strategy to analyze the sgRNA sequences for the double sgRNA library. (C) Empirical Bayes fit of the null distribution of the constructed test statistic using the R package locfdr. (D) Correlation of sequenced sgRNA counts in library-transduced CamES cells at D0, Tuj1-hCD8+ cells and Tuj1-hCD8− cells after hCD8 sorting.

FIG. 28A-1H shows (A) Ngn1 and Foxo1 induced MEF neuron cells express MAP2, Tuj1 and NeuN, synapsin, and GABA 14 days after infection. Scale bar, 100 μm. (B) Bar graph showing the percentage of Tbr1-positive neurons (Tbr1+) and GABA-positive neurons (GABA+) out of total neurons. (C) Ngn1 and Foxo1 induced perinatal TTF neuron cells express MAP2, Tuj1, and NeuN, synapsin, and GABA 26 days after infection. Scale bar, 100 μm. (D) Inward sodium currents were evoked from induced neurons, and application of 500 nM TTX inhibited these currents. (E) Outward potassium currents were evoked from an induced neurons, and application of 5 mM tetraethylammonium (TEA) inhibited these currents. (F) Spontaneous action potentials recorded from an induced neuron (left panel). Application of 100 nM TTX blocked the action potentials (middle panel). Washout of TTX reversed the blockade (right panel). ((G) Representative traces of evoked excitatory spontaneous postsynaptic currents (EPSCs) recorded from an induced neuron (left panel). Application of 30 μM DNQX (6,7-dinitroquinoxaline-2,3-dione), an AMPA/kainate glutamate receptor antagonist, blocked the response of EPSCs (middle panel). Washout of DNQX reversed the blockade (right panel). (H) Representative traces of evoked EPSCs recorded from an induced neuron (left panel). Application of 30 μM BIC (Bicuculline), a GABA receptor antagonist, slightly increased the frequency and amplitude of EPSCs (middle panel). Washout of BIC reversed the increase (right panel). G and H, Cells were recorded at a holding potential (Vh) of −60 mV. Error bars indicate±s.d. of cell counts.

FIG. 29 shows representative images of Tuj1 staining of MEFs infected with different gene combinations. Scale bar, 100 μm.

### DEFINITIONS

As used herein the term “stem cell” (“SC”) refers to cells that can self-renew and differentiate into multiple lineages. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. Stem cells may be derived, for example, from embryonic sources (“embryonic stem cells”) or derived from adult sources. For example, U.S. Pat. No. 5,843,780 to Thompson describes the production of stem cell lines from human embryos. PCT publications WO 00/52145 and WO 01/00650 (herein incorporated by reference in their entireties) describe the use of cells from adult humans in a nuclear transfer procedure to produce stem cell lines.

Examples of adult stem cells include, but are not limited to, hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

As used herein, the term “totipotent cell” refers to a cell that is able to form a complete embryo (e.g., a blastocyst).

As used herein, the term “pluripotent cell” or “pluripotent stem cell” refers to a cell that has complete differentiation versatility, e.g., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), a pluripotent cell, even a pluripotent embryonic stem cell, cannot usually form a new blastocyst.

As used herein, the term “induced pluripotent stem cells” (“iPSCs”) refers to a stem cell induced from a somatic cell, e.g., a differentiated somatic cell, and that has a higher potency than said somatic cell. iPS cells are capable of self-renewal and differentiation into mature cells.

As used herein, the term “multipotent cell” refers to a cell that has the capacity to grow into a subset of the mammalian body's approximately 260 cell types. Unlike a pluripotent cell, a multipotent cell does not have the capacity to form all of the cell types.

As used herein, the term “progenitor cell” refers to a cell that is committed to differentiate into a specific type of cell or to form a specific type of tissue.

As used herein, the term “embryonic stem cell” (“ES cell” or ESC”) refers to a pluripotent cell that is derived from the inner cell mass of a blastocyst (e.g., a 4- to 5-day-old human embryo), and has the ability to yield many or all of the cell types present in a mature animal.

As used herein the term “feeder cells” refers to cells used as a growth support in some tissue culture systems. Feeder cells may, for example, embryonic striatum cells or stromal cells.

As used herein, the term “chemically defined media” refers to culture media of known or essentially-known chemical composition, both quantitatively and qualitatively. Chemically defined media is free of all animal products, including serum or serum-derived components (e.g., albumin).

### DETAILED DESCRIPTION

Provided herein are compositions and methods for identifying and using stem cell differentiation regulation factors. For example, in some embodiments, provided herein are compositions and methods for identifying stem cell differentiation regulation factors using marker gene expression libraries. Also provided herein are compositions and methods for generating differentiated and induced cells lines and uses of such cell lines.

The RNA-guided microbial endonuclease CRISPR (clustered regularly interspaced short palindromic repeat)/Cas9 (CRISPR associated protein 9) system was recently repurposed as a tool for sequence-specific gene editing and transcriptional regulation (Cho et al., 2013 Nat. Biotechnol. 31, 230-232; Cong et al., 2013 Science 339, 819-823; Fu et al., 2014 Nat. Biotechnol. 32, 279-284; Jinek et al. Science 337, 816-821, 2012; Mali et al., 2013b Science 339, 823-826; Qi et al., 2013 Cell 152, 1173-1183; Ran et al., 2015 Nature 520, 186-191; Yu et al., 2015 Cell Stem Cell 16, 142-147). The nuclease-dead Cas9 (dCas9) fused with transcription activator domains allows endogenous genes activation, leading to CRISPR activation (CRISPRa) methods (Chavez et al., 2015 Nat. Method. 12, 326-328; Cheng et al., 2013 Cell Res. 23, 1163-1171; Gilbert et al., 2013 Cell 154, 442-451; Hilton et al., 2015 Nat. Biotechnol. 33, 510-517; Konermann et al., 2015 Nature 517, 583-588; Maeder et al., 2013 Nat. Method. 10, 977-979; Mali et al., 2013a Nat. Biotechnol. 31, 833-838; Perez-Pinera et al., 2013 Nat. Method. 10, 973-976; Tanenbaum et al., 2014 Cell 159, 635-646; Zalatan et al., 2015 Cell 160, 339-350). Previous work demonstrated that CRISPR activation of endogenous genes allowed, in principle, somatic cell reprogramming and directed cell differentiation (Black et al., 2016 Cell Stem Cell 19, 406-414; Chakraborty et al., 2014 Stem Cell Reports 3, 940-947; Chavez et al., 2015 Nat. Method. 12, 326-328; Wei et al., 2016 Sci. Rep. 6, 19648). However, since these studies relied on using a mixture of multiple sgRNAs for activating a single gene and inducing differentiation, applying these methods for large-scale activation screening has been a major challenge.

Unlike cell growth phenotypes that entail a dropout live-or-dead process, cell fate determination is a dynamic, stochastic process that often generates a heterogeneous cell population with diverse phenotypes (e.g., non-dropout) (Hanna et al., 2009 Nature 462, 595-601; Johnston and Desplan, 2010 Annu. Rev. Cell Dev. Biol. 26, 689-719). This imposes another challenge to simply perform dropout screens that distinguish lineage specification processes from spontaneous differentiation events. Furthermore, because developmental programs are highly dependent on the expression level of endogenous genes (Niwa et al., 2000 Nat. Genet. 24, 372-376; Papapetrou et al., 2009 Proc. Natl. Acad. Sci. USA 106, 12759-12764), gain-of-function screens that allow very efficient gene activation (comparable to cDNA overexpression) while covering a broad range of expression offer more promise for identifying candidate genes driving cell lineages. To date, two reports used CRISPRa for cell growth-based dropout screens (Gilbert et al., 2014 Cell 159, 647-661; Konermann et al., 2015 Nature 517, 583-588). However, the application of CRISPRa screens for the systematic inference of cell fate determination has not yet been established.

Experiments described herein overcame these challenges by developing a CRISPR activation (CRISPRa)-mediated gain-of-function screening approach to identify transcription factors (TFs) important for stem cell fate determination. An enhanced CRISPRa system was developed in mouse embryonic stem (ES) cells that efficiently activates endogenous genes and drives cell lineage differentiation. A single sgRNA was sufficient to induce neuron or muscle differentiation. Based on the system, a large-scale sgRNA library (>50,000 sgRNA) was used to target all putative endogenous TF genes (˜800) and a small set of noncoding RNA genes (50). Targeting a single gene using multiple sgRNAs (>60 sgRNA per gene) allowed activating each gene to a broad range of expression levels. A CRISPRa dropout screen was used to identify genes that promote stem cell self-renewal, as well as a non-dropout screen for inducing neural differentiation. The top gene hits were validated using individual sgRNAs, and it was observed that all hits could maintain self-renewal. For neural differentiation, it was confirmed that 19 out of top 20 gene hits could induce efficient neural differentiation. For both screens, the lists of gene hits include known TF factors and those TFs and noncoding RNAs that are not previously related to self-renewal maintenance or neural differentiation. Different identified TFs preferentially induced different types of neurons. Deep sequencing and functional analysis of a few gene hits (Mlxip for self-renewal and Jun for neural differentiation) confirmed their functions for driving desired cellular processes.

Thus, the compositions and methods provide herein allow for the identification of the relevant factors necessary, sufficient, and/or useful for controlling differentiation of stem cells into any desired fat. The transcription factors identified herein and identifiable using the compositions and methods described herein provide target and reagents for differentiation of cells an provide the cells made therefrom that find use as research tools, drug screening targets, and therapeutics (e.g., via cell transplantation into a host).

The CRISPRa gain-of-function screens and stem cell libraries described herein find use in research, therapeutic, and screening applications to determine differentiation factors for a variety of stem cells. The differentiation factors identified further find use in stem cell differentiation for research, screening, and clinical applications.

### 1. Identification of Differentiation Factors

As described herein, embodiments of the present disclosure provide compositions and methods for identifying stem cell differentiation regulation factors. In some embodiments, the methods utilize a modified pluripotent or multipotent (e.g., stem cell) line. The present disclosure is not limited to particular cell lines. Examples include, but are not limited iPSC, embryonic stem cells, adult stem cells, and the like.

In some embodiments, the CRISPR activation system comprises a dCas9 construct under the transcriptional control of a first promoter. In some embodiments, the dCas9 is fused to a peptide epitope. In some embodiments, the activation system further comprises a VP64 transactivation domain under the transcriptional control of a second promoter. In some embodiments, the VP64 transactivation domain is fused to a peptide that specifically binds to the peptide epitope. In some embodiments, the activation system further comprises a selection marker under the transcriptional control of a third promoter. In some embodiments, each of the first, second, and third promoters are different than each other.

In some embodiments, cell lines for determination of differentiation regulation factors are pluripotent cells modified with a dead Cas9/transactivator activation system. For example in some embodiments, cells comprise a nuclease dead Cas9 (dCas9). In some embodiments, the dCas9 is fused to a signal activation component (e.g., a plurality of peptide epitopes as described in Tanenbaum et al., (2014). Cell 159, 635-646; herein incorporated by reference in its entirety). In some embodiments, the cell lines further comprise a single chain variable chain antibody fragment specific for the peptide epitope fused to a tranactivator domain (e.g., VP64; See e.g., Beerli et al., Proc Natl Acad Sci USA. 1998 Dec 8; 95(25): 14628-14633; herein incorporated by reference in its entirety) and a transactivator polypeptide. In some embodiments, the activation components are provided on a vector (e.g., retroviral vector, adenoviral viral vector, adeno-associated vector, lentiviral vector, etc.). In some embodiments, cells further overexpress endogenous Brn2 (e.g., via an sgRNA that targets activation of Brn2).

In some embodiments, the cells lines are next contacted with a plurality of sgRNAs (e.g., targeting cell differentiation regulation factors). In some embodiments, sgRNAs target transcription factors or non-coding RNAs (e.g., lincRNAs). In some embodiments, more than one (e.g., at least 10, 20, 30, 40, 50, 60, 70, 80, 90, or 100) sgRNAs specific for each differentiation factor are utilized. In some embodiments, sgRNAs are provided on vectors (e.g., retroviral vector, adenoviral viral vector, adeno-associated vector, lentiviral vector, etc.). In some embodiments, cells are further contacted with a plurality of non-targeting sgRNAs (e.g., to serve as negative controls). In some embodiments, a double CRISPR screen is performed using dual-sgRNA-constructs comprising two (or more) sgRNAs to screen for interactions between multiple cell differentiation factors in combination.

In some embodiments, the method further comprises contacting the cell differentiation factors with a fibroblast or other cell line and identifying cell differentiation factors that promote transdifferentiation of the fibroblast cell line. In some embodiments, the fibroblast cell line is contacted with combinations of two or more cell differentiation factors. In some embodiments, the cell differentiation factors that promote differentiation are combinations of Ngn1+Brn2, Ezh2+Brn2, Mecom+Ezh2, Ngn1+Ezh2, or Ngn1+Foxo1.

In some embodiments, the method comprises or further comprises the step of performing a CRISPR gene repression screen. For example, in some embodiments, the CRISPR repression screen comprises: a) contacting a pluripotent cell that expresses dCas9 fused to a transcription repressor domain (e.g., KRAB) with a plurality of sgRNAs specific for repression of a plurality of cell differentiation factors; b) sorting the library to identify cells that retain pluripotency or differentiate; and c) identifying cell differentiation factors that induce or prevent differentiation of said pluripotent cells. In some embodiments, the CRISPR repression screen and the CRISPR activation screen are performed in the same or different pluripotent cells. In some embodiments, the CRISPR repression screen and the CRISPR activation screen are performed simultaneously using vectors comprising a first sgRNA specific for activation of a first cell differentiation factor and a second sgRNA specific for repression of a second cell differentiation factor.

The resulting gene activation library from CRISPR activation and/or repressor cells are then further analyzed as described below. For example, in some embodiments, following delivery of sgRNAs, cells are cultured and cells that retain pluripotency or differentiate are identified. In some embodiments, cells are sorted based on the presence or absence of differentiation or pluiptency markers.

In some embodiments, in order to identify regulation factors for pluipotency, cells are cultured under conditions that do not inhibit differentiation (e.g., in media lacking inhibitors of GSK3 and ERK pathways). In some embodiments, pluripotent cells are sorted by identifying and selecting (e.g., using flow cytometry) cells that express SSEA1 after culture.

In some embodiments, cells that differentiate are identified by sorting for cells that express differentiation markers specific to the final cell type. For example, in some embodiments, cells that differentiate into neuronal cells are identified by sorting for cells that express Tuj1.

In some embodiments, cell differentiation factors are activated and analyzed in pairs or groups (e.g., as described in Example 2 below) in order to identify combined effects of between different factors.

In some embodiments, after selection, cell differentiation regulation factors are identified by identifying sgRNAs that persist in the sorted cells. In some embodiments, sequencing (e.g., deep sequencing) is used to identify sgRNAs. In some embodiments, sequencing methods further comprises comparing the level of said sgRNAs to the level of non-targeting sgRNAs.

In deep sequencing, a high number of replicates of each sequencing read (e.g., at least 10, 20, 30, 40, 50, or 100) are used to improve accuracy. The present disclosure is not limited to a particular sequencing technique. Exemplary sequencing techniques are described below. A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

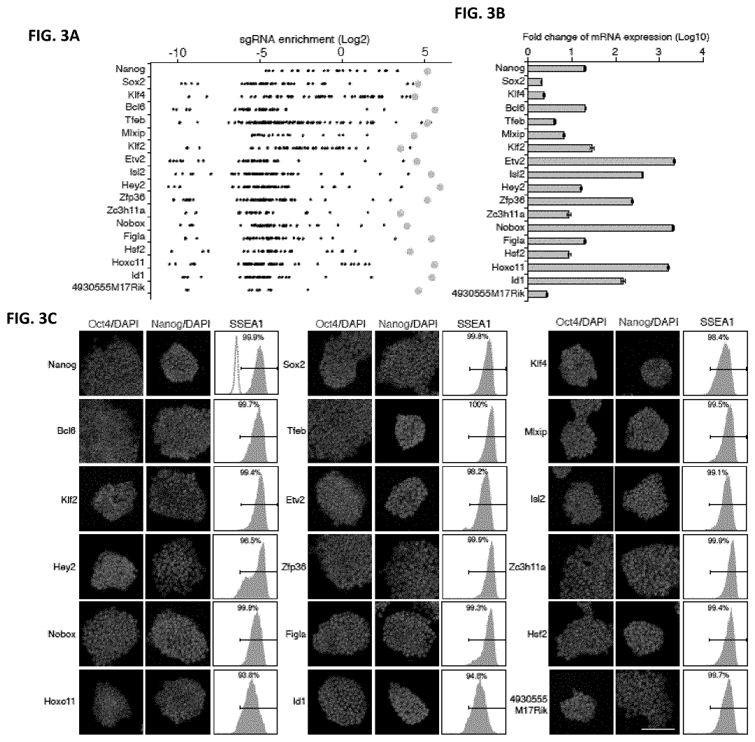

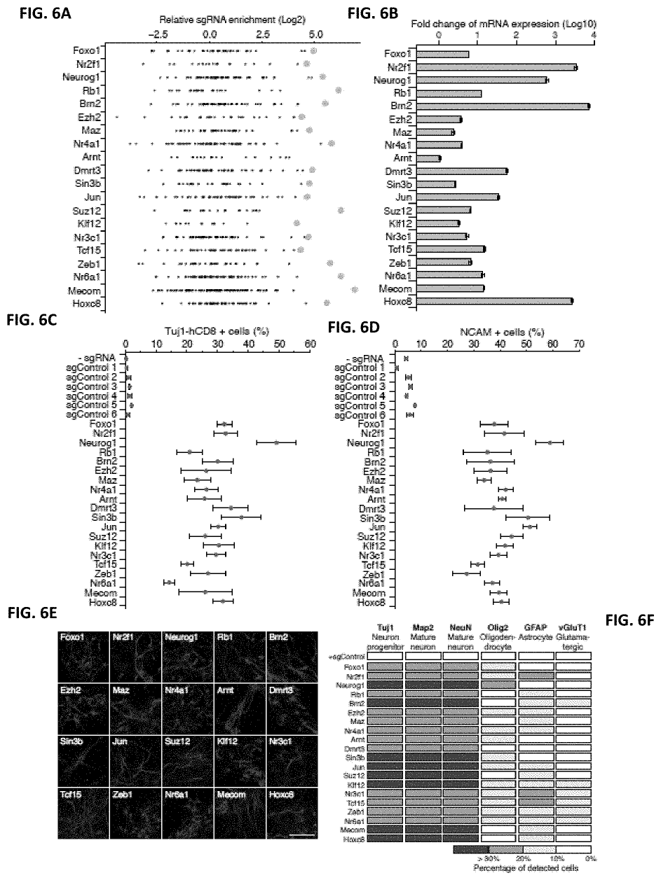

Exemplary cell regulation factors indicative of cells that retain pluripotency or differentiate are described in the Figures and Tables herein. For example, in some embodiments, cell transcription factors that retain pluripotency are one or more of the regulation factors shown in FIG. 3 or Table 3 and cell differentiation factors that are associated with differentiation into neuronal cells are one or more of the regulation factors shown in FIG. 6 or Table 4.

The cell differentiation factors identified using the described methods find use in a variety of applications. Exemplary uses are described herein.

### II. Cell Lines and Libraries and Uses Thereof

In some embodiments, the present disclosure provides cells lines, kits, and systems for use in the described methods. For example, in some embodiments, provided herein are libraries of modified pluripotent cells as described above. For example, in some embodiments, the cells comprise a dCas9 construct under the transcriptional control of a first promoter. In some embodiments, the dCas9 is fused to a peptide epitope. In some embodiments, the cells comprise a VP64 transactivation domain under the transcriptional control of a second promoter. In some embodiments, the VP64 transactivation domain is fused to a peptide that specifically binds to the peptide epitope. In some embodiments, the cells comprise a selection marker under the transcriptional control of a third promoter. In some embodiments, each of the first, second, and third promoters are different than each other.

In some embodiments, cells express i) nuclease dead Cas9 fused to a plurality of peptide epitopes; ii) a single chain variable chain antibody fragment specific for said peptide epitope fused to a VP64 tranactivator domain; and iii) a transactivator polypeptide.

In some embodiments, the cell lines described herein find use in screening (e.g., drug screening) and research applications as described below.

In some embodiments, provided herein are kits and systems comprising the cell lines described herein. In some embodiments, kits and systems further comprise a plurality of sgRNAs specific for activation of pluripotent cell differentiation factors. In some embodiments, the kit or system comprises one or more sgRNAs (e.g., 10 or more, 100 or more, 1000 or more, or 5000 or more) described in Table 13 (e.g., SEQ ID NOs:586-8317).

In some embodiments, kits and systems further comprise reagents for analysis of one or more properties of the cell lines (e.g., pluripotency or differentiation), reagents for sequencing the cells to identify the presence of sgRNAs, reagents for further downstream analysis (e.g., molecular analysis, toxicity screening, drug screening, or cellular activity assays), or computer software and computer systems for analyzing data.

### III. Differentiation Methods

In some embodiments, the present disclosure provides compositions and methods for differentiating cells into multipotent or specific cell types. The present disclosure is not limited to particular target cell types. Examples include, but are not limited to, epithelial cells (e.g., exocrine secretory epithelial cells, hormone secreting cells (e.g., islet cells), keratinizing epithelial cells (e.g., skin cells), central nervous system cells (e.g., neuronal cells), blood cells, and organ cells.

In some embodiments, differentiation is induced by increasing expression of cellular regulation factors identified using the methods described herein. In some embodiments, expression is induced by exogenously introduced differentiation genes. In one embodiment, the exogenously introduced gene may be expressed from a chromosomal locus different from the endogenous chromosomal locus of the gene. Such chromosomal locus may be a locus with open chromatin structure, and contain gene(s) dispensible for a somatic cell. In other words, the desirable chromosomal locus contains gene(s) whose disruption will not cause cells to die. Exemplary chromosomal loci include, for example, the mouse ROSA 26 locus and type II collagen (Col2a1) locus (See Zambrowicz et al., 1997) The exogenously introduced pluripotency gene may be expressed from an inducible promoter such that their expression can be regulated as desired.

In some embodiments, the exogenously introduced gene is transiently transfected into cells, either individually or as part of a cDNA expression library. The cDNA library is prepared by conventional techniques. Briefly, mRNA is isolated from an organism of interest. An RNA-directed DNA polymerase is employed for first strand synthesis using the mRNA as template. Second strand synthesis is carried out using a DNA-directed DNA polymerase which results in the cDNA product. Following conventional processing to facilitate cloning of the cDNA, the cDNA is inserted into an expression vector such that the cDNA is operably linked to at least one regulatory sequence. The choice of expression vectors for use in connection with the cDNA library is not limited to a particular vector. Any expression vector suitable for use in mammalian cells is appropriate. In one embodiment, the promoter which drives expression from the cDNA expression construct is an inducible promoter. The term regulatory sequence includes promoters, enhancers and other expression control elements. Exemplary regulatory sequences are described in Goeddel: Gene Expression Technology: Methods in Enzymology, Academic Press, San Diego, Calif. (1990). For instance, any of a wide variety of expression control sequences that control the expression of a DNA sequence when operatively linked to it may be used in these vectors to express cDNAs. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

In some embodiments, the CRISPR activation and/or repression system is expressed from an inducible promoter. The term “inducible promoter”, as used herein, refers to a promoter that, in the absence of an inducer (such as a chemical and/or biological agent), does not direct expression, or directs low levels of expression of an operably linked gene (including cDNA), and, in response to an inducer, its ability to direct expression is enhanced, Exemplary inducible promoters include, for example, promoters that respond to heavy metals (CRC Boca Raton, Fla. (1991), 167-220; Brinster et al. Nature (1982), 296, 39-42), to thermal shocks, to hormones (Lee et al. P.N.A.S. USA (1988), 85, 1204-1208; (1981), 294, 228-232; Klock et al. Nature (1987), 329, 734-736; Israel and Kaufman, Nucleic Acids Res. (1989), 17, 2589-2604), promoters that respond to chemical agents, such as glucose, lactose, galactose or antibiotic.

A tetracycline-inducible promoter is an example of an inducible promoter that responds to an antibiotics. See Gossen et al., 2003. The tetracycline-inducible promoter comprises a minimal promoter linked operably to one or more tetracycline operator(s). The presence of tetracycline or one of its analogues leads to the binding of a transcription activator to the tetracycline operator sequences, which activates the minimal promoter and hence the transcription of the associated cDNA and the expression of CRISPR activation and/or repression system. Tetracycline analogue includes any compound that displays structural homologies with tetracycline and is capable of activating a tetracycline-inducible promoter. Exemplary tetracycline analogues includes, for example, doxycycline, chlorotetracycline and anhydrotetracycline.

In some embodiments, expression of cell differentiation factors is induced via activating sgRNAs as described herein (e.g., Example 1). One or more sgRNAs are introduced into a pluripotent cell that expresses a CRISPR activation system (e.g., those described herein or other suitable system).

In some embodiments, differentiation is induced via small molecules that active expression or activity of cell differentiation genes or downstream signaling partners.

In some embodiments, cells are cultured under conditions that promote differentiation. In some embodiments, cultures are adherent cultures, e.g., the cells are attached to a substrate. The substrate is typically a surface in a culture vessel or another physical support, e.g. a culture dish, a flask, a bead or other carrier. In some embodiments, the substrate is coated to improve adhesion of the cells and suitable coatings include laminin, poly-lysine, poly-ornithine and gelatin. In some embodiments, the cells are grown in a monolayer culture or in suspension or as balls or clusters of cells. At higher densities, cells may begin to pile up on each other, but the cultures are essentially monolayers or begin as monolayers, attached to the substrate.

Cells differentiated using the methods described herein find use in a variety of research, screening, and clinical applications. In some embodiments, cells are used to prepare antibodies and cDNA libraries that am specific for the differentiated phenotype. General techniques used in raising, purifying and modifying antibodies, and their use in immunoassays and immunoisolation methods are described in Handbook of Experimental Immunology (Weir & Blackwell, eds.), Current Protocols in Immunology (Coligan et al., eds.); and Methods of Immunological Analysis (Masseyeff et al., eds., Weinheim: VCH Verlags GmbH). General techniques involved in preparation of mRNA and cDNA libraries are described in RNA Methodologies: A Laboratory Guide for Isolation and Characterization (R. E. Farrell, Academic Press, 1998); cDNA Library Protocols (Cowell & Austin, eds., Humana Press); and Functional Genomics (Hunt & Livesey, eds., 2000). Relatively homogeneous cell populations are particularly suited for use in drug screening and therapeutic applications.

In some embodiments, the cells generated by methods provided herein or the above-described cell lines are used to screen for agents (e.g., small molecule drugs, peptides, polynucleotides, and the like) or environmental conditions (such as culture conditions or manipulation) that affect the cells. Particular screening applications relate to the testing of pharmaceutical compounds in drug research. Assessment of the activity of candidate pharmaceutical compounds generally involves combining the cells with the candidate compound, determining any change in the morphology, marker phenotype, or metabolic activity of the cells that is attributable to the compound (compared with untreated cells or cells treated with an inert compound), and then correlating the effect of the compound with the observed change. Any suitable assays for detecting changes associated with test agents may find use in such embodiments. The screening may be done, for example, either because the compound is designed to have a pharmacological effect on specific cell types, because a compound designed to have effects elsewhere may have unintended side effects, or because the compound is part of a library screen for a desired effect. Two or more drugs can be tested in combination (by combining with the cells either simultaneously or sequentially), to detect possible drug-drug interaction effects. In some applications, compounds are screened for cytotoxicity.

In some embodiments, methods and systems are provided for assessing the safety and efficacy of drugs that act upon the differentiated cells, or drugs that might be used for another purpose but may have unintended effects upon the cells. In some embodiments, cells described herein find use in high throughput screening (ITS) applications. In some embodiments, a HTS screening platform is provided (e.g., cells and plates) that allows for the rapid testing of large number (e.g., 1×10³, 1×10⁴, 1×10⁵, 1×10⁶ (or more) of agents (e.g., small molecule compounds, peptides, etc.).

In some embodiments cells generated using methods and reagents described herein are utilized for therapeutic delivery to a subject (e.g., a subject with a disease or other condition). Cells may be placed directly in contact with subject tissue or may be otherwise sealed or encapsulated (e.g., to avoid direct contact). In embodiments in which cells are encapsulated, exchange of factors, nutrients, gases, etc. between the encapsulated cells and the subject tissue is allowed. In some embodiments, cells are implanted/transplanted on a matrix or other delivery platform.

If appropriate, cells are co-administered with one or more pharmaceutical agents or bioactives that facilitate the survival and function of the transplanted cells.

Support materials suitable for use for purposes of the present disclosure include tissue templates, conduits, barriers, and reservoirs useful for tissue repair. In particular, synthetic and natural materials in the form of foams, sponges, gels, hydrogels, textiles, and nonwoven structures, which have been used in vitro and in vivo to reconstruct or regenerate biological tissue, as well as to deliver chemotactic agents for inducing tissue growth, are suitable for use in practicing the methods of the present disclosure. See, for example, the materials disclosed in U.S. Pat. Nos. 5,770,417, 6,022,743, 5,567,612, 5,759,830, 6,626,950, 6,534,084, 6,306,424, 6,365,149, 6,599,323, 6,656,488, U.S. Published Application 2004/0062753 A1, U.S. Pat. Nos. 4,557,264 and 6,333,029.

Cells generated with methods and reagents herein may be implanted as dispersed cells or formed into implantable clusters. In some embodiments, cells are provided in biocompatible degradable polymeric supports; porous, permeable, or semi-permeable non-degradable devices; or encapsulated (e.g., to protect implanted cells from host immune response, etc.). Cells may be implanted into an appropriate site in a recipient. Suitable implantation sites depend on the cell type and may include, for example, the brain, spinal cord, skin, liver, natural pancreas, renal subcapsular space, omentum, peritoneum, subserosal space, intestine, stomach, or a subcutaneous pocket.

In some embodiments, cells or cell clusters are encapsulated for transplantation into a subject. Encapsulation techniques are generally classified as microencapsulation, involving small spherical vehicles, and macroencapsulation, involving larger flat-sheet and hollow-fiber membranes (Uludag, H. et al. Technology of mammalian cell encapsulation. Adv Drug Deliv Rev. 2000; 42: 29-64, herein incorporated by reference in its entirety).

Methods of preparing microcapsules include those disclosed by Lu M Z, et al. Biotechnol Bioeng. 2000, 70: 479-83; Chang T M and Prakash S, Mol Biotechnol. 2001, 17: 249-60; and Lu M Z, et al., J. Microencapsul. 2000, 17: 245-51; herein incorporated by reference in their entireties. For example, microcapsules may be prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56; herein incorporated by reference in its entirety). In some embodiments, microcapsules are based on alginate, a marine polysaccharide (Sambanis. Diabetes Technol. Ther. 2003, 5: 665-8; herein incorporated by reference in its entirety) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

In some embodiments, cells generated using methods and reagents described herein are microencapsulated for transplantation into a subject (e.g., to prevent immune destruction of the cells). Microencapsulation of cells provides local protection of implanted/transplanted cells from immune attack (e.g., along with or without the use of systemic immune suppressive drugs). In some embodiments, cells and/or cell clusters are microencapsulated in a polymeric, hydrogel, or other suitable material, including but not limited to: poly(orthoesters), poly(anhydrides), poly(phosphoesters), poly(phosphazenes), polysaccharides, polyesters, poly(lactic acid), poly(L-lysine), poly(glycolic acid), poly(lactic-co-glycolic acid), poly(lactic acid-co-lysine), poly(lactic acid-graft-lysine), polyanhydrides, poly(fatty acid dimer), poly(fumaric acid), poly(sebacic acid), poly(carboxyphenoxy propane), poly(carboxyphenoxy hexane), poly(anhydride-co-imides), poly(amides), poly(ortho esters), poly(iminocarbonates), poly(urethanes), poly(organophasphazenes), poly(phosphates), poly(ethylene vinyl acetate), poly(caprolactone), poly(carbonates), poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide, polystyrene, polysaccharides, alginate, hydroxypropyl cellulose (HPC), N-isopropylacrylamide (NIPA), polyethylene glycol, polyvinyl alcohol (PVA), polyethylenimine, chitosan (CS), chitin, dextran sulfate, heparin, chondroitin sulfate, gelatin, etc., and their derivatives, co-polymers, and mixtures thereof. In some embodiments, cells are microencapsulated in an encapsulant comprising or consisting of alginate. Cells may be embedded in a material or within a particle (e.g., nanoparticle, microparticle, etc.) or other structure (e.g., matrix, nanotube, vesicle, globule, etc.). In some embodiments, microencapsulating structures are modified with immune-modulating or immunosuppressive compounds to reduce or prevent immune response to encapsulated cells. For example, in some embodiments, cells are encapsulated within an encapsulant material (e.g., alginate hydrogel) that has been modified by attachment of an immune-modulating agent (e.g., the immune modulating chemokine, CXCL12 (also known as SDF-1). In some embodiments, such an immune modulating agent is a T-cell chemorepellent and/or a pro-survival factor.

In some embodiments, cells generated using methods and reagents described herein are macroencapsulated for transplantation into a subject. Macroencapsulation of cells, for example, within a permeable or semi-permeable chamber, provides local protection of implanted/transplanted cells from immune attack (e.g., along with or without the use of systemic immune suppressive drugs), prevents spread of cells to other tissues or areas of the body, and/or allows for efficient removal of cells. Suitable devices for macroencapsulation include those described in, for example, U.S. Pat. No. 5,914,262; Uludag, et al., Advanced Drug Delivery Reviews, 2000, pp. 29-64, vol. 42, herein incorporated by reference in their entireties.

Other encapsulation (micro or macro) devices and methods may find use in embodiments described herein. For example, methods and devices described in U.S. Pub No. 20130209421, U.S. Pat. No. 8,785,185, each of which are herein incorporated by reference in their entireties, are within the scope of embodiments described herein.

### IV. Differentiation Factors

As described above and in the examples below, a number of new transcription factor and other regulatory factors involved in the regulating the differentiation processes have been discover using the screening methods described herein. These factors find use in generating stem cells or differentiated cells have desired properties for use in research, drug screening, and therapeutic applications.

In some embodiments, individual or combinations of these factors are used to induce differentiation in a stem cell to obtain differentiated cells or multipotent cells of a particular lineage (e.g., neural stem cells). In some embodiments, such factor are introduced exogenously to stem cells in vitro or in vivo (e.g., via expression vector, etc). In some embodiments, endogenous factors are up or down regulated by providing activators or inhibitors of endogenous expression.

In some embodiments, individual or combinations of these factors are used to induce differentiation in a somatic cell (e.g., fibroblast, neuronal cell, etc).

In some embodiments, individual or combinations of these factors are used to maintain or induce pluripotency in a cell line. In some embodiments, such factor are introduced exogenously to stem cells or somatic cells in vitro or in vivo (e.g., via expression vector, etc). In some embodiments, endogenous factors are up or down regulated by providing activators or inhibitors of endogenous expression.

In some embodiments, one or more of the markers described in Tables 3 and 4 are targeted. In some embodiments, provided herein are one or more sgRNAs (e.g., 10 or more, 100 or more, 1000 or more, or 5000 or more) described in Table 13 (e.g., SEQ ID NOs:586-8317) for use in targeting the described markers.

In some embodiments, provided herein are cell generated by such methods and the use of such cells, for example, in drug screening, diagnostic, and therapeutic indications.

Where transcription factors are introduced as peptides, in some embodiments they are complexed with cell membrane permeable peptides (e.g., Tat protein, penetratin, etc.) to facilitate entry into target cells.

### EXPERIMENTAL

### Example 1

### CRISPR Activation Screens Identify Genes Promoting Self-Renewal and Neuronal Differentiation of Stem Cells

### Methods

sgRNA Library Construction

The oligo library was PCR amplified, gel purified and ligated to the linearized backbone vector (pSLQ1373) digested with BstXI and BlpI using In-Fusion cloning (Clontech).

Cell Culture

E14 mouse ES cells and CamES cells were maintained on gelatin coated tissue culture plates with basal medium (50% Neurobasal, 50% Dulbecco modified Eagle medium (DMEM) Ham's nutrient mixture F12, 0.5% NEAA, 0.5% Sodium Pyruvate, 0.5% GlutaMax, 0.5% N2, 1% B27, 0.1 mM β-mercaptoethanol and 0.05 g/L bovine albumin fraction V; all from Thermo Fisher Scientific) supplemented with LIF (Millipore) and 2i (Stemgent), Human embryonic kidney (HEK293T) cells (ATCC) were cultured in 10% fetal bovine serum (Thermo Fisher Scientific) in DMEM (Thermo Fisher Scientific).

Lentiviral Production

HEK293T cells were seeded at ˜30% confluence one day before transfection. Lentivirus were produced by cotransfecting with pHR plasmids and encoding packaging protein vectors (pMD2.G and pCMV-dR8.91) using TransIT-LT1 transfection reagents (Mirus). Viral supernatants were collected 3 days after transfection and filtered through 0.45 μm strainer. Supernatant was used for transduction immediately or kept at −80° C. for long-term storage.

High-Throughput Pooled Screening

Screens were performed in two independent replicates for both self-renewal and neural differentiation. For both screens, 10⁸ CamES cells were transduced with the pooled lentiviral library with an MOI of 0.3, treated with puromycin, and cultured in specified medium. After a period of time indicated for each screen, cells were harvested and FACS/MACS sorted. Deep sequencing was performed to profile the sgRNA counts in each sample, and computationally analyzed to infer top sgRNA and gene hits.

Plasmid Design and Construction

To clone sgRNA vectors, the optimized sgRNA expression vector (pSLQ1373) was linearized and gel purified (Chen et al., 2013 Cell 155, 1479-1491). New sgRNA sequences were PCR amplified from pSLQ1373 using different forward primers and a common reverse primer, gel purified and ligated to the linearized pSLQ1373 vector using In-Fusion cloning (Clontech). Primers used to construct individual sgRNAs are shown in Table 1. To change the promoter of scFv-sfGFP-VP64, the EF1α and PGK promoters were PCR amplified, gel purified, and ligated to linearized pSLQ1504 using In-Fusion cloning (Clontech).

sgRNA Library Design

Putative transcription factor (TF) genes were selected according to the TRANSFAC database, and TSS (transcription start site) for each gene was determined using the Gencode and refFlat databases. All possible transcripts were selected if multiple TSSs existed for a gene. All sgRNAs targeting was −3 kb to 0 relative to TSS. Using the CRISPR-era algorithm (Liu et al., 2015 Bioinformatics 31, 3676-3678), the targeting sequences of sgRNAs adjacent to an NGG PAM (protospacer adjacent motif) were computed, starting with a G (for more efficient U6 promoter activity) with a length of 20 bp. The sgRNAs containing homopolymers spanning greater than 3 nucleotides (nt) were discarded. To avoid off-target effects, sgRNA sequences alignment to the mouse genome was computed using the short read aligner Bowtie, and those with less than 2 mismatches with another genomic region were excluded. Furthermore, sgRNA sequences that contained certain restriction sites (BstXI and XhoI) were also removed. sgRNAs with a GC content between 30% and 70% were used. An average of about 60 sgRNAs were selected for each target gene. Sequences for non-targeting negative control sgRNAs were generated using a randomized mouse gene TSS region and selected using the same rules as described above.

sgRNA Library Construction

The oligonucleotide pool was synthesized by Custom Array. The oligo library was PCR amplified, gel purified and ligated to the linearized pSLQ1373 digested with BstXI and BlpI using in-Fusion cloning.

Construction of the CamES Cell Line

Mouse ES cells were co-transduced with multiple lentiviral constructs that expressed dCas9-SunTag from a TRE3G promoter, scFV-sfGFP-VP64 from the EF1a or PGK promoter, and rtTA from the EF1a promoter. After adding Doxycycline, polyclonal cells were sorted by flow cytometry using a BD FACS Aria2 for GFP+ and mCherry+ cells. After verification of gene activation using a sgBrn2, monoclonal cells were further sorted, and one efficient cell line was selected as CamES cells.

Construction of the Tuj-1-hCD8 CamES Cell Line

Construction of CRISPR/Cas9 vector for Tuj1 knockin. The pX330-derived pSLQ1654 encoding the nuclease Cas9 and an optimized sgRNA sequence was first linearized by a BbsI digest and gel purified. Two primers sgTuj-1 F and sgTuj-1 R were phosphorylated, annealed, and ligated to the linearized vector pSLQ1654 to generate pSLQ1654-sgTuj1. sgTuj-1 F: caccgcccaagtgaagttgctcgcagc (SEQ ID NO:378). sgTuj-1 R: aaacgctgcgagcaacttcacttgggc (SEQ ID NO:379).

Construction of DNA template. The Tuj1-IRES-hCD8 vector (pSLQ1760) was assembled with three fragments (5′ homologous arm of Tuj1, IRES-hCD8 and 3′ homologous arm of Tuj1) and a modified pUC19 backbone vector by using Gibson Assembly Master Mix (New England Biolabs). Both 5′ and 3′ homology arms were PCR amplified from the genomic DNA extracted from mouse ES cells with Herculase 11 Fusion DNA polymerase (Agilent). The IRES-hCD8 was PCR amplified from pSLQ1729 (gift from Wendell Lim). The backbone vector was linearized by digestion with PmeI and Zra1. All DNA fragments and the backbone vector were gel purified followed by a Gibson assembly reaction. Primers: 5′ homologous arm F: aaagtgccacctgacactcagtccLagatgtcgtgcgg. 5′ (SEQ ID NO:380) homologous arm R: tcacttgggcccctgggct (SEQ ID NO:381). IRES-human CD8 F: caggggcccaagtgaactagtaaaattcgcccctctccctc (SEQ ID NO:382). IRES-human CD8 R: cagctgcgagcaactttaacctgcaaaaagggagcagtuaaagg (SEQ ID NO:383). 3′ homologous arm F: agttgctcgcagctggggt (SEQ ID NO:384). 3′ homologous arm R: agctggagaccgttttttctgactgactggatacagggcat (SEQ ID NO:385).

Electroporation and clonal Tuj1-hCD8 CamES cells: 2.5 μg pSLQ1654-sgTuj1, 12.5 μg Tuj1-1RES-hCD8 template DNA in 100 μL. Nucleofector solution (Amaxa) were electroporated into 1×10⁶ CamES cells using program A-030. Both plasmids were maxiprepped using the Endofree Maxiprep Kit (Qiagen). After 3 days of culture, sorted single cells were seeded in a 96-well plate with one cell per well. All clonal cell lines were analyzed using PCR and sequencing (Yu et al., 2015 Cell 16, 142-147).

Quantitative RT-PCR

Cells were harvested using Accutase (STEMCELL), and total RNA was isolated using the RNeasy Plus Mini Kit (QIAGEN), according to manufacturer's instructions. Reverse transcription was performed using iScript cDNA Synthesis kit (Bio-Rad). Quantitative PCR reactions were prepared with iTaq Universal SYBR Green Supermix (Bio-Rad). Reactions were run on a LightCycler thermal cycler (Bio-Rad). Primers used are summarized in Table 2.

High-Throughput Pooled Self-Renewal Screening

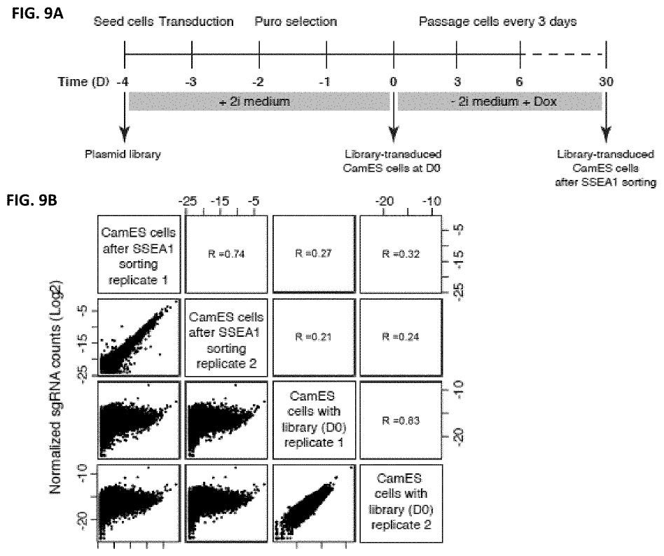

Screens were performed in two independent replicates. For both screens. 10⁸ CamES cells were transduced with the pooled lentiviral library with an MOI of 0.3 on day −3. On day −2, CamES cells were treated with puromycin (Invitrogen, 1 μg/mL) in basal medium supplemented with LIF and 2i. After 48 hours of puromycin selection, cells were harvested as the day 0 sample. Another 10⁸ CamES cells with the same treatment were passaged for 10 times under the basal medium supplemented with LIF and Doxycycline (Invitrogen, 100 ng/mL), without 2i. Cells were passaged every 3 days. After 30 days, cells were harvested, stained with mouse anti-SSEA1 (BD, 1:50), and FACS sorted using BD FACS Aria2 as SSEA1+ sample (FIG. 9A). For the individual sgRNA validation experiments, a similar protocol was used, except that the CamES cells were infected with a high MOL. Top 100 hits are summarized in Table 3.

High-Throughput Pooled Neural Differentiation Screening

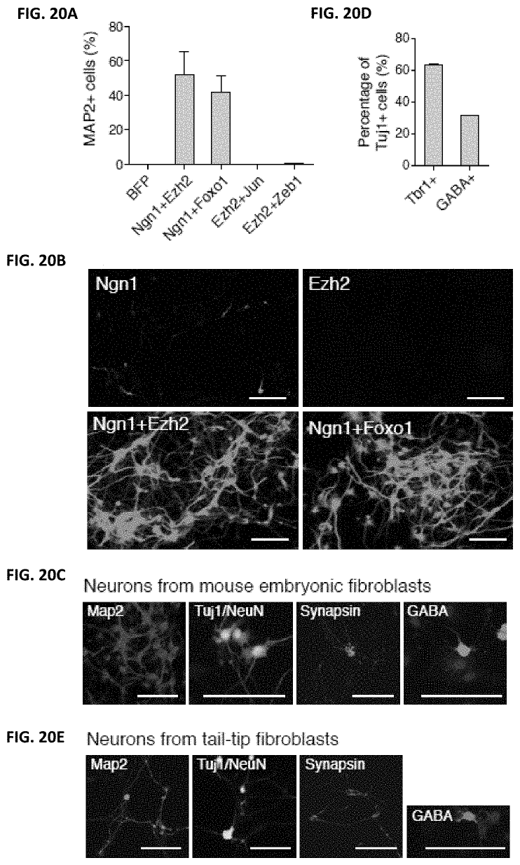

The neural differentiation screens were performed as two independent replicates. For both screens, 10⁸ CamES cells were seeded at 40,000 cells/cm² density at day −1. Cells were transduced with pooled lentiviral sgRNA library with an MOI of 0.3 at day 0 in basal medium supplemented with LIF and 2i. At day 1, puromycin was added at 1 μg/mL in ES2N medium (Millipore) with Doxycycline for another 24 hours. Fresh ES2N medium was changed with Doxycycline every day starting day 2. On day 12, cells were harvested and sorted for hCD8+ and hCD8− cells using EasySep human CD8 isolation kit (STEMCELL Technologies) (FIG. 20F). For the individual sgRNA validation experiments, a similar protocol except that CamES cells were cultured in basal medium seeded at 5,500 cells/cm² after puromycin selection and transduced with a high MOI was used. Top 100 hits are summarized in Table 4.

Flow Cytometry Analysis

Cells were harvested, washed, and adjusted to a concentration of 10⁶ cells/mL, in ice cold PBS with 2% FBS. Cells were stained and incubated with diluted primary antibodies at 4° C. for 30 mins in Eppendorf tubes. After staining, cells were washed three times by centrifugation at 400 g for 5 mins and resuspended in 500 μL to 1 mL in ice cold PBS. Cells were kept in dark on ice and analyzed using BD Accuri C6 Cytometer.

Immunocytochemistry

Experiments were performed on cells seeded on plate (IBIDI) that had been coated with gelatin (0.1%) overnight at 37° C. Cells were washed twice with PBS, fixed in 4% Paraformaldehyde (Wako) for 15 mins at room temperature, permeabilized and blocked with 0.1% Triton X-100, 5% donkey serum in PBS (blocking buffer) for 1 h at room temperature. After three times wash with PBS, cells were incubated with primary antibodies. The following primary antibodies with indicated dilution in blocking buffer were used: Rabbit anti-Oct4 (Santa Cruz, 1:200), Rabbit anti-Nanog (Abcam, 1:500), Mouse anti-Tuj1 (Covance, 1:1000), Rabbit anti-Map2 (Cell Signaling Technology, 1:200), Rabbit anti-NeuN (Abcam, 1:1000), Rabbit anti-vGluT1 (Synaptic Systems, 1:200). Rabbit anti-GFAP (Dako, 1:500), Rabbit anti-Olig-2 (Millipore, 1:500) Cells were incubated with primary antibodies at 4° C. for overnight, then washed three times with PBS. After staining with corresponding secondary antibodies in blocking buffer for 1 hour at room temperature, cells were washed three times with PBS and stained with DAPI (Vector Labs) for 5 mins. Washed cells were examined using a Nikon Spinning Disk Confocal microscope with TIRF.

Electrophysiology

External bath solution for whole cell patch clamp recordings contains (in mM) 140 NaCl, 5 KCl, 2 cacl₂, 2 MgC₂, 20 HEPES, and glucose 10, pH 7.4. Action potentials were recorded current-clamp while sodium and potassium currents were recorded under voltage clamp. The internal pipette solution contained (in mM): 123 K-gluconate, 10 KCl, 1 MgCl2, HEPES, 1 EGTA, 0.1 CaCl2, 1 MgATP, 0.3 Na4GTP and glucose 4, pH 7.2. For current clamp experiments, currents were injected to keep membrane potentials around −65 mV, and action potentials were elicited by stepwise current injections.

Western Blot

Samples were collected with NP40 buffer with protease inhibitor and phosphatase inhibitor, and boiled in 1×SDS loading buffer, separated by SDS-PAGE gels, and transferred onto a nitrocellulose (NC) membrane, which was blocked with 5% non-fat dry milk and incubated with primary antibodies at 4° C. overnight. Rabbit anti-Jun antibody (Cell Signaling Technology, 1:1000), rabbit anti-β-actin antibody (Cell Signaling Technology, 1:5000), rabbit anti-phospho-Jun antibody (Cell Signaling Technology. 1:1000) were used as primary antibodies. HRP-conjugated donkey anti-rabbit IgG (Jackson ImmunoResearch, 1:5000) were used as secondary antibodies. Signals were detected using SuperSignal West Femto Maximum Sensitivity Substrate (Thermo Scientific). β-actin was used as a loading control.

Differentiation of Mouse ES Cells Through Embryoid Body Formation

The sgKlf2- and sgMlxip-transduced CamES cells were trypsinized, plated on ultralow attachment plates, and cultured in Knockout DMEM supplemented with 10% FBS, without Doxcycline. After 6 days, aggregated cells were collected and seeded onto gelatin-coated plates. Four days later, cells were fixed and stained with markers for three germ layers.

RNA-Seq

CamES cells were transduced with individual sgRNAs, expanded, and differentiated after 2 days of puromycin selection in 6 well plates. Total RNA was purified using RNeasy Plus Mini Kit (Qiagen). Libraries were prepared using TruSeq Stranded mRNA LT Sample Prep kit (Illumina) according to the manufacturer's instructions. Samples were combined and purified using Ampure XP Agencourt beads (Beckman Coulter) and sequenced on a Hi-Seq 4000 (Illumina), to generate paired-end 150 bp reads. Each sample was sequenced to an average depth of 40 million reads.

Reads were mapped with kallisto (Bray et al., 2016 Nature biotechnology 34, 525-527) to the provided GRCm38 downloaded from bio math at Berkley. Normalized gene expression and differentially expressed genes were estimated using sleuth (Pimentel et al., 2016 bioRxiv) and DESeq2 (Love et al., 2014 Genome Biol 15, 550) for the self-renewal and neural data, respectively. Gene ontology analysis was performed using the Bioconductor package gage (Luo et al., 2009 BMC Bioinformatics 10, 161). AP-1 targets were defined as genes that have an AP-1 consensus binding motif (Biddie et al., 2011 Mol Cell 43, 145-155; Rauscher et al., 1988 Genes & Development 2, 1687-1699; Shaulian and Karin, 2002 Nat Cell Biol 4, E131-E136; Zhou et al., 2005 DNA Research 12, 139-150) within 500 bases upstream of the TSS.

Bioinformatic Analysis of sgRNA and Gene Hits

Data processing was conducted with custom scripts. Reads were mapped allowing for a mismatch for the first and last base pair of the spacer, which uniquely identified sgRNA.

Each sample was normalized by the total read count. This gave a frequency for each sgRNA:

$${f}_{sgRNA} = \frac{sgRNA counts}{\sum sgRNA counts}$$

For the self-renewal screen, in each condition (CamES cells and SSEA+ cells), frequency for each sgRNA was averaged across replicates. sgRNA with less than 20 counts at time 0 were discarded. The sgRNA enrichment (E_{sg}) was calculated as the log 2 fold change from the average time 0 frequency to the average SSEA+ frequency.

For the neuronal differentiation screen, the paired Tuj1-hCD8+ and Tuj1-hCD8− were used to compute the enrichment scores. Specifically, frequencies were computed as above, sgRNA with less than 1 count in the Tuj1-hCD8− library was discarded. Enrichment for each sgRNA in each replicate was calculated as the log 2 fold-change from the Tuj1-hCD8− sample to the Tuj1-hCD8− libraries. Enrichment was averaged across replicates and used as E_{sg} in subsequent analysis.

For each gene, an enrichment score (ES_{gene}) was calculated from the sgRNA enrichment above, as follows. An unnormalized enrichment score (E_{gene.top3}) was calculated by averaging F_{sg} for the 3 sgRNA with highest E_{sg}. E_{gene.top3} was normalized by the distribution of nontargeting sgRNA as follows (Gilbert et al., 2014 Cell 159, 647-661).

Suppose a gene had N targeting sgRNA. Then, 10000 bootstrap samples of size N were drawn from the nontargeting sgRNA. For each sample of size N, E_{sample.top3} was computed as above. This gave an empirical estimate of the distribution of E_{gene.top3} if the all the sgRNA targeting that gene had been negative control sgRNA. For the final, normalized gene enrichment score (ES_{gene}), the unnormalized enrichment score was divided by the 0.9 quantile of thie smpirical distribution:

$${ES}_{gene} = \frac{{E}_{gene , top 3}}{{quantile}_{samples} ( {E}_{sample , top 3} , 0.9 )}$$

After ranking genes by ES, the most enriched sgRNA for each gene was selected to subsequently validate.

### Results

Generation of CRISPRa Mouse Embryonic Stem Cells for Single sgRNA-Mediated Gene Activation and Cell Fate Control

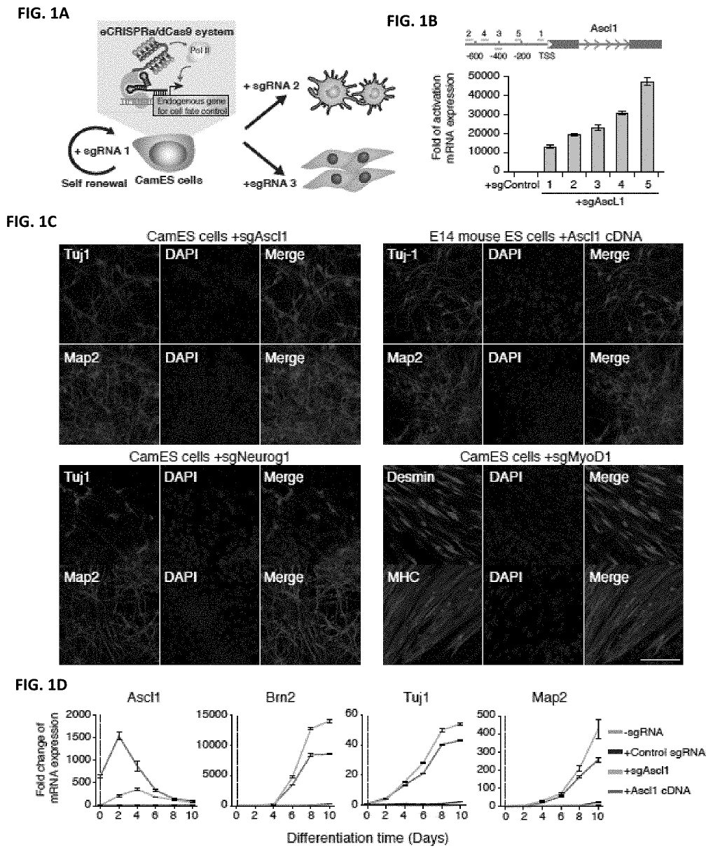

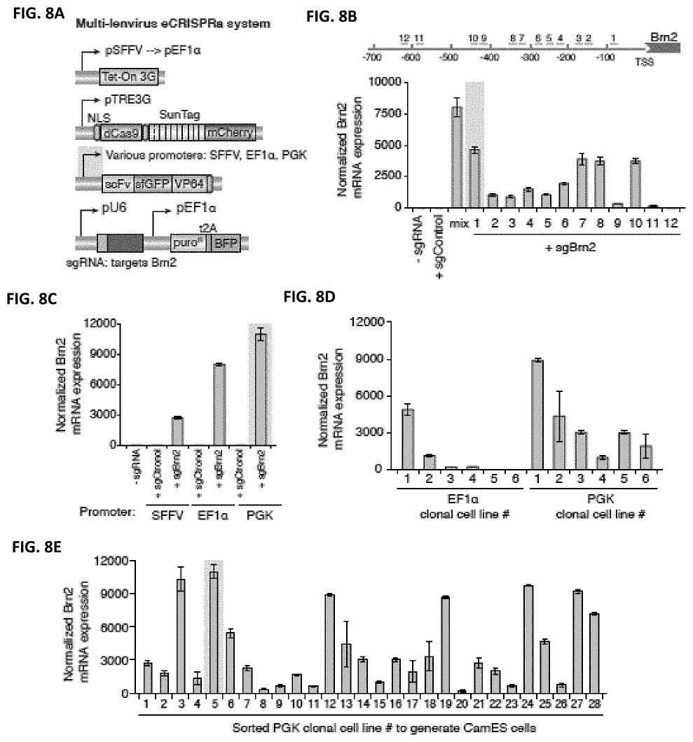

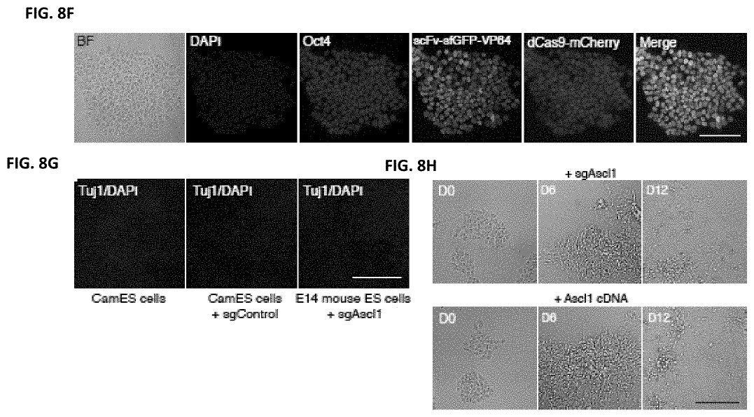

Single sgRNA-mediated efficient endogenous gene activation is useful for large-scale pooled screens of sophisticated cell differentiation phenotypes (FIG. 1A). To establish such a highly efficient CRISPRa system, a reported CRISPRa system based on a polypeptide array, SunTag, was used (Tanenbaum et al., (2014). Cell 159, 635-646). A panel of individual or mixed sgRNAs was used to activate endogenous Brn2 (FIGS. 8A and 8B), a gene driving neuron formation in mouse ES cells (Sokolik et al., 2015 Cell Systems 1, 117-129). Mixed sgRNAs showed better activation compared to individual sgRNAs, whereas none of them induced neural differentiation.

The dCas9-SunTag system contains two components, a SunTag polypeptide domain fused to dCas9 and a VP64 transactivator domain fused to a single chain fragment variable (scFv). It was investigated whether their expression ratio was a key factor determining the activation efficiency. To facilitate fine-tuning their ratio, each component was cloned onto a lentiviral vector (FIG. 8A). The dCas9-Suntag fusion was expressed using a Doxycycline (Dox)-inducible promoter pTRE3G, and the SFFV promoter was replaced with an EF1a promoter for Tet-On 3G transactivator expression, as silenced SFFV activity was observed during ES cell differentiation. It was tested if promoters (PGK, EF1a, and SFFV) with different strengths driving scFv-VP64 fusion could lead to various activation efficiencies. It was observed the PGK promoter exhibited best endogenous Brn2 expression using both bulk and clonal cells (FIGS. 8C and 8D). By tuning the stoichiometry ratio between the two components, an enhanced CRISPRa (eCRISPRa) system with better activation of endogenous genes was obtained.

Twenty eight clonal cell lines with the PGK promoter were sorted, and one cell line (#5) showing best Brn2 activation was obtained, which was named CamES (CRISPR-activating mouse ES) cells (FIG. 8E). It was confirmed that this cell line could be stably cultured in ES cell conditions, while maintaining stem cell morphology and pluripotency and expressing eCRISPRa components over a long-term passage (FIG. 8F). It was determined if CamES cells allowed efficient activation of another gene, Asc11, using a single sgRNA. All 5 Asc11 sgRNAs showed strong activation (>10,000 fold) compared to using a control sgRNA (FIG. 1B). In addition, the activation efficiency varied among 5 sgRNAs, showing that a broad range of gene activation can be achieved.

It was next tested if this promoted neural differentiation (Chanda et al., 2014 Stem Cell Reports 3, 282-296). Using a single sgAsc11, robust differentiation of CamES cells into a neuronal phenotype was observed at day 8, which stained positively for the neuronal markers Tuj1 (class III beta-tubulin) and Map2 (Microtubule-associated protein 2) (FIG. 1C). All negative controls (CamES cells without sgRNA, CamES cells with non-target control sgRNA, and E14 mouse ES cells with sgAsc11) showed no neural differentiation morphology or neural marker expression, confirming neurons were indeed induced by eCRISPRa-mediated target gene activation. Another neural transcription factor, Neurog1 (Velkey and O'Shea, 2013 Dev Dyn. 242, 230-253), was tested with a single sgRNA, and similarly observed neuron formation (FIG. 1C). The cell line also showed efficient skeletal muscle differentiation using a single sgRNA activating MyoD1 (FIG. 1C) (Shani et al., 1992 Symp. Soc. Exp. Biol. 46, 19-36). These experiments together demonstrate that CamES cells allow single sgRNA-mediated endogenous gene activation and cell differentiation.

The CamES cells activating endogenous Asc11 were compared with overexpression of exogenous Asc11 cDNA for neural differentiation. A similar neuronal phenotype was observed using the two approaches (FIG. 1C). It was found that cells using two systems showed similar morphogenetic features characterized by the formation of neural rosettes after 6 days of differentiation and extensive neurite outgrowth between days 8-12 (FIG. 9H). Though overexpression of exogenous cDNA showed higher total Asc11 expression, CRISPRa-mediated endogenous Asc11 activation exhibited comparable or even better neural differentiation as seen by the fold change of other neural markers Brn2, Tuj1, and Map2 over a 10-day differentiation process (FIG. 1D). The data demonstrated that modulating endogenous genes is a better strategy for directed cell differentiation compared to cDNA expression. Taken together, these results showed that the CamES cells were able to induce high-level endogenous gene expression using only a single sgRNA for controlling cell fate.

CamES Cells Allow an eCRISPRa-Mediated Dropout Screen to Identify Transcription Factors that Maintain Self-Renewal

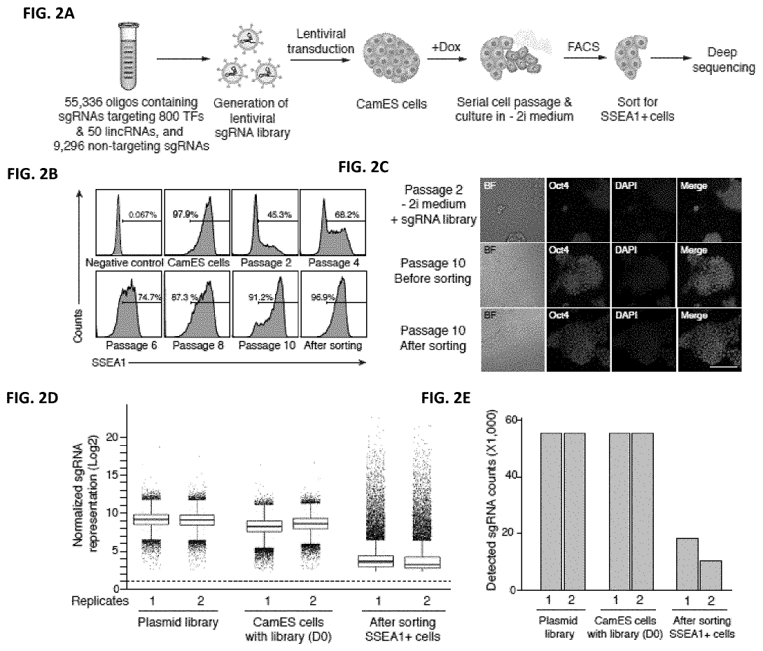

CamES cells were used as an unbiased screening platform to identify key factors among the set of all putative transcription factors that direct cell fate determination. Initial studies focused on factor contributing to the maintenance of ES cell self-renewal. An sgRNA library targeting all putative TFs (˜800) and a small set of lincRNAs (long intergenic noncoding RNAs) (˜50) was generated. Multiple sgRNA (60 sgRNAs per gene on average) were designed to target each gene to cover a broad range of gene activation. An additional 9,296 non-targeting negative control sgRNAs were included. Altogether, a library with a total of 55,336 sgRNAs was generated (FIG. 2A).

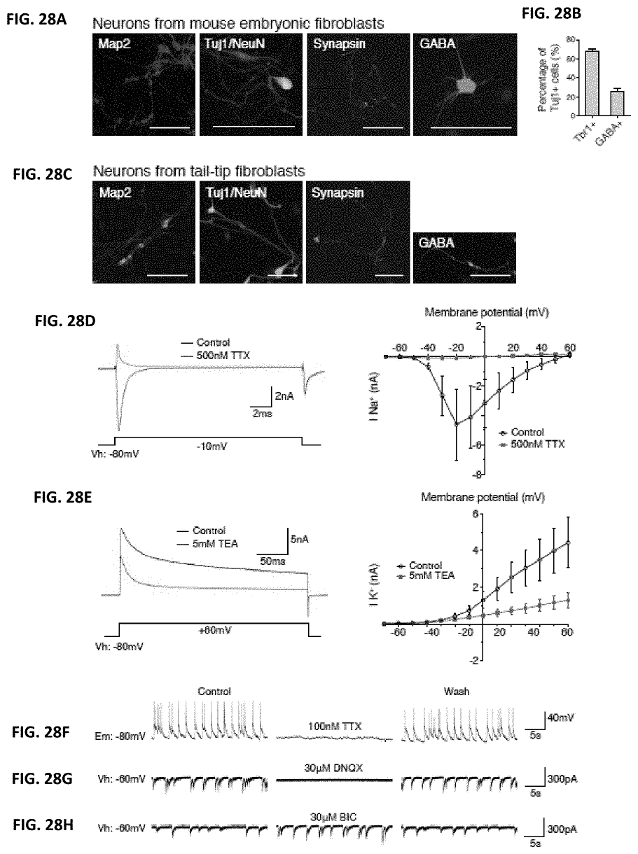

The sgRNA library was introduced into CamES cells as a gain-of-function screen to study stem cell self-renewal. Self-renewal of mouse ES cells in serum-free conditions requires simultaneous inhibition of the GSK3 and ERK pathways, which is typically achieved by using two small molecule inhibitors (2i) (Ying et al., 2008 Nature 453, 519-523). It was determined whether activating transcription factors could functionally rescue the loss of 2i to support self-renewal over a long period of time. To do this, the lentiviral sgRNA library was transduced into CamES cells, cultured the transduced cells in −2i medium, and passaged every three days (FIGS. 2A and 9A). For library transduction, MOI (multiplicity of Infection) was kept below 0.3 such that the majority of cells were transduced only with a single sgRNA. Over half of cells quickly lost pluripotency markers (SSEA1 and Oct4) and initiated spontaneous differentiation within two passages post library transduction (FIGS. 28 and 2C). Repeated passaging of cells removed most differentiated cells, while the SSEA1+ population gradually increased over time, providing a dropout screen. After 10 passages, SSEA1+ cells were sorted using FACS (flow cytometry activated sorting), which further increased SSEA1+ cell percentage to 96.9% (FIG. 2B). The sorted cells showed mouse ES cell morphology and were Oct4+, confirming maintenance of pluripotency (FIG. 2C).

To identify genes whose gain-of-function maintains self-renewal of ES cells, deep sequencing was used to read out the sgRNA representation (FIG. 2A). The overall distribution of sgRNAs from samples collected from the original plasmid library, CamES cells with sgRNA library at day 0, and sorted SSEA1+ cells after passage 10 were compared (FIG. 9A). Only a small fraction of sgRNAs were detected after sorting compared to the plasmid library and day 0 samples (FIGS. 2D and 2E), indicating an efficient selection process.

Gene-level enrichment scores were obtained by considering the enrichment of the top three sgRNAs targeting each gene and normalizing by the empirical distribution of the non-targeting sgRNA. A good correlation was obtained between both sgRNA enrichment and gene-level scores across independent library transductions (FIG. 9B).

Validation of Top Enriched sgRNAs Promoting Long-Term Maintenance of Self-Renewal in ES Cells

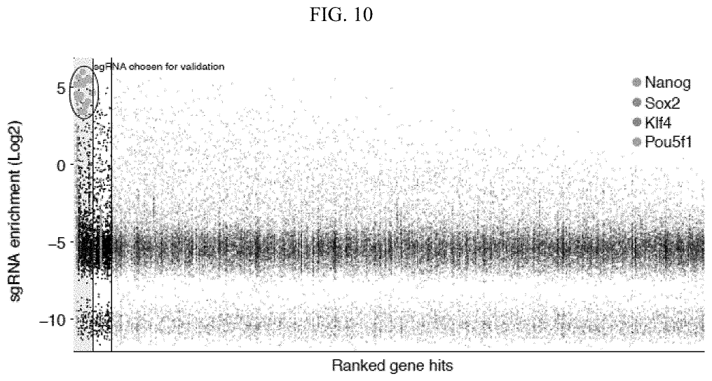

Using the non-targeting sgRNA normalized gene scoring method, all detected sgRNAs and their targeting genes were ranked (FIG. 10). For each gene, the majority of designed sgRNAs were depleted, implying either most genes had no function in self-renewal or the depleted sgRNAs were unable to sufficiently activate gene expression for functional genes. Major pluripotency factors such as Nanog, Sox2, Klf4, and Oct4 appeared as top enriched hits, consistent with previous works showing their critical roles in maintaining stem cell self-renewal (Chambers et al., 2003 Cell 113, 643-655; Masui et al., 2007 Nat. Cell Biol. 9, 625-635; Mitsui et al., 2003 Cell 113, 631-642; Niwa et al., 2000 Nat. Genet. 24, 372-376; Zhang et al., 2010 J. Biol. Chem. 285, 9180-9189).

The most enriched sgRNAs of the top 18 genes were selected for validation (FIG. 3A). The 18-gene list contained pluripotency genes (Klf2 and Id1) (Jiang et al., 2008 Nat. Cell Biol. 10, 353-360; Yeo et al., 2014 Cell Stem Cell 14, 864-872; Ying et al., 2003a Cell 115, 281-292), lineage specific genes (Etv2 and Isl2) (Koyano-Nakagawa et al., 2012 Stem Cells 30, 1611-1623; Thaler et al., 2004 Neuron 41, 337-350), and one lincRNA gene (4930555M17Rik). For validation, 18 individual sgRNAs were constructed and transduced into CamES cells. Six individual non-targeting sgRNAs were included as negative controls. None of the negative control sgRNAs was able to maintain stem cell self-renewal in −2i medium condition beyond passage 2.

Quantitative PCR results confirmed activation of target genes by each sgRNA (FIG. 3B). All 18 sgRNAs maintained stem cell morphology and expressed pluripotency markers Oct4, Nanog, and SSEA1 after culturing in −2i condition over 30 days (FIG. 3C). Notably, 9 out of 18 validated genes (Mlxip, Etv2, Zc3h11a, Zfp36, Isl2, Tfeb, Fig1a, Hsf2, and Hoxc11) are not previously annotated for maintenance of pluripotency and self-renewal. The high rate of validated true hits indicates that the screening method provides an effective dropout screen of genes promoting self-renewal and maintaining pluripotency.

Deep Sequencing and Functional Validation Confirmed the Function of Positive Hits for Self-Renewal Maintenance

sgMlxip was chosen to explore its role in promoting self-renewal. The MLXIP protein forms a heterodimer with MLX (Max-like protein X) and modulates transcriptional regulation in response to cellular glucose levels (Stoltzman et al., 2008 Proc. Natl. Acad. Sci. USA 105, 6912-6917), and its function related to ES cell self-renewal is unknown.

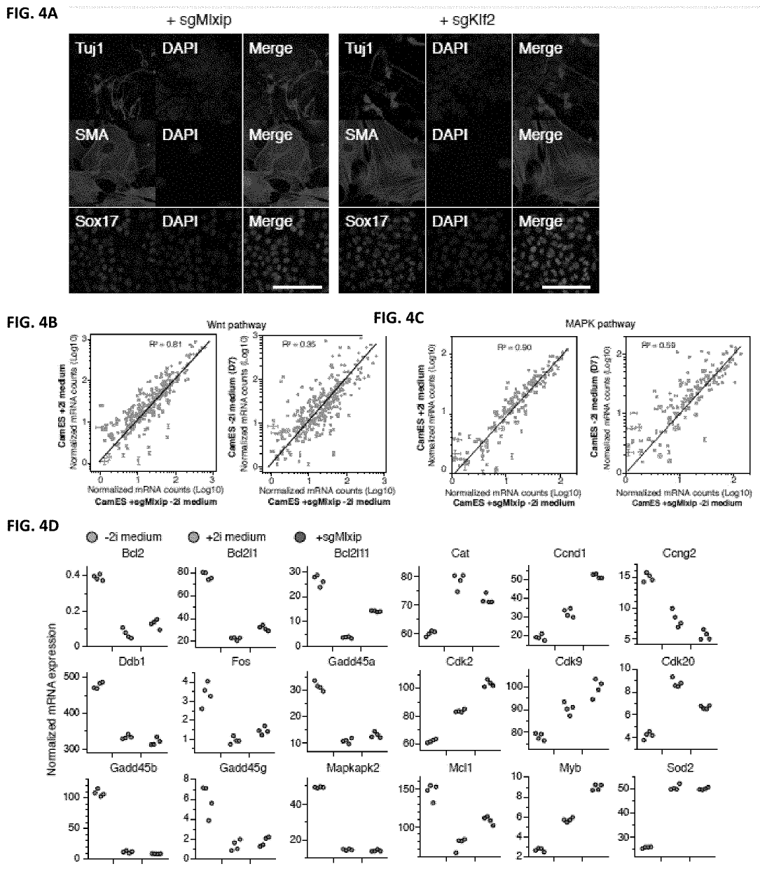

The developmental potential of CamES +sgMlxip cells cultured in −2i conditions for generating the three germ layers was evaluated using CamES +sgKlf2 as a comparison. After removal of Dox to switch of eCRISPRa activity, spontaneous differentiation of both samples in serum-based medium via embryoid body formation generated cells representative of ecdoderm (Tuj1+), mesoderm (SMA+), and endoderm (Sox17+) lineages (FIG. 4A). This confirmed the differentiation potential of these cells cultured in −2i medium.

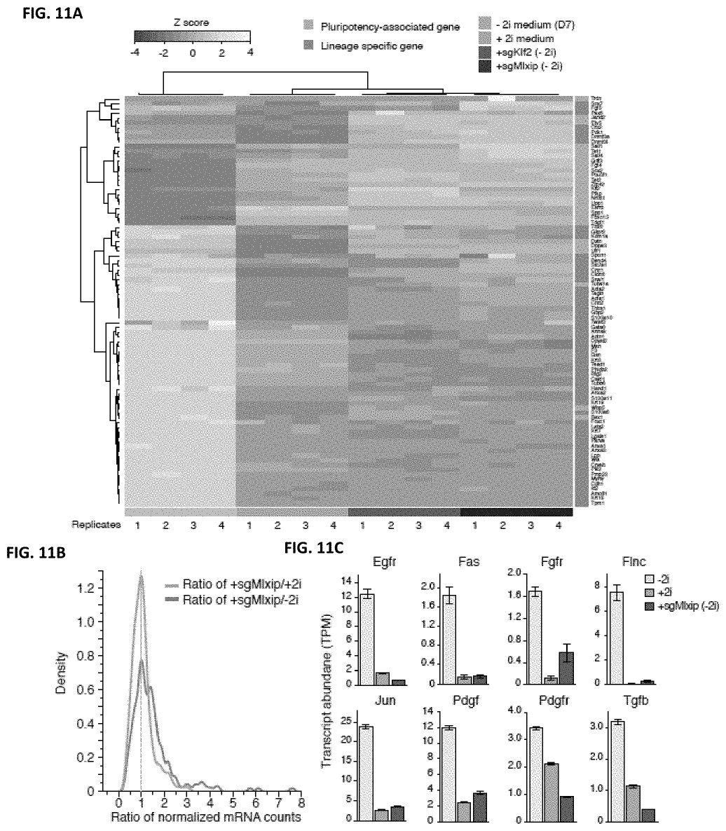

RNA-seq analysis was performed on CamES +sgMlxip and CamES +sgKlf2 cells cultured in −2i conditions, and compared to CamES cells cultured with or without 2i. Both samples exhibited high mRNA expression for most pluripotency genes and low expression for most lineage specific genes, with a pattern similar to ES cells cultured in 2i medium and distinct from cells without 2i (FIG. 11A), indicating that the CamES +sgMlxip and CamES +sgKlf2 cells maintained a similar gene expression profile as the undifferentiated stem cells in 2i medium.

The 2i cocktail contains two small molecules that maintain pluripotency by inhibiting GSK3 (CHIR99021) and MEK1/2 (PD0325901) (Ying et al., 2008 Nature 453, 519-523). Via activation of the Wnt pathway and inhibition of the MAPK pathway, the 2i molecules inhibit differentiation while promoting proliferation of ES cells. The RNA-seq gene expression profiles for the Wnt and MAPK pathways were compared among the samples. For the Wnt pathway genes, CamES-sgMlxip cells correlated well with CamES cells in +2i medium (R²=0.81), while poorly with CamES cells in −2i medium (R²=0.35) (FIG. 4B). A different ratio distribution of corresponding gene expression between +sgMlxip/+2i and +sgMlxip/−2i was found (FIG. 11B) (Zhang et al., 2013 Stem Cells 31, 2667-2679).

Similar results were observed for the MAPK pathway: there was a good correlation between CamES +sgMlxip and CamES +2i samples (R²=0.91), compared to a poor correlation between CamES-sgMlxip and CamES-2i (R²=0.59). Gene expression related to the MAPK pathway showed a similar pattern at the transcript level in both CamES +sgMlxip and CamES +2i cells. For example, inhibition of Jun, a major transcription factor of the MAPK pathway, was observed in both CamES +sgMlxip and CamES +2i cells, as well as inhibition of other MAPK related genes (EGF, FAS, FGF, PDGF and TGFb) (FIG. 11C). These results together indicate that CamES +sgMlxip cells possess similar Wnt and MAPK pathway activities as CamES +2i cells.

The PI3K pathway, which is important in the regulation of ES cell pluripotency and proliferation (Yu and Cui, 2016 Development 143, 3050-3060), was also investigated. The CamES +sgMlxip cells also showed a similar expression pattern as CamES +2i cells (FIG. 4D). For example, PI3K-related genes such as Fos, Mapkapk2, Gadd45b, and Gadd45g were downregulated in both CamES +sgMlxip and CamES +2i cells, while Ccnd1, Cdk2, Cdk9, and Sod2 were similarly upregulated (FIG. 4D). The PI3K gene expression further confirms the similarity between CamES +sgMlxip cells and ES cells cultured in 2i medium.

In summary, both functional tests and gene expression indicate that true positive hits identified using the CRISPRa screening method maintain self-renewal of stem cells.

Engineered CamES Cells Allow an eCRISPRa-Mediated Non-Dropout Screen to Identify Key Factors Promoting Neural Differentiation

A eCRISPRa gain-of-function screen was performed to identify TFs that promote the dynamic, complex neural differentiation process. Transcription factor-mediated lineage specification is heterogeneous and stochastic: unlike in the dropout screen, a desired differentiated cell type may only represent a small subset of the total population; and spontaneous differentiation may generate the desired cell type even when a non-functional factor is present.

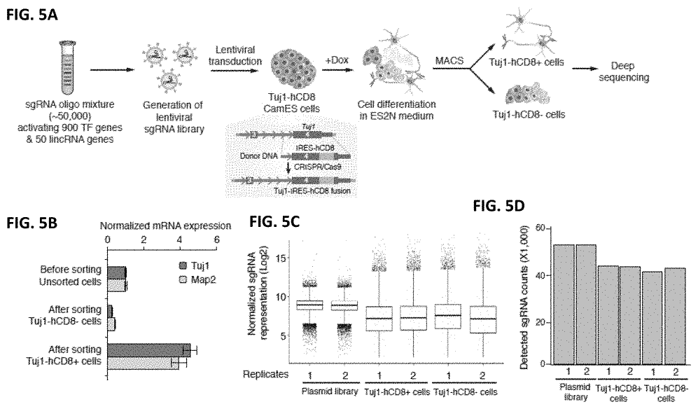

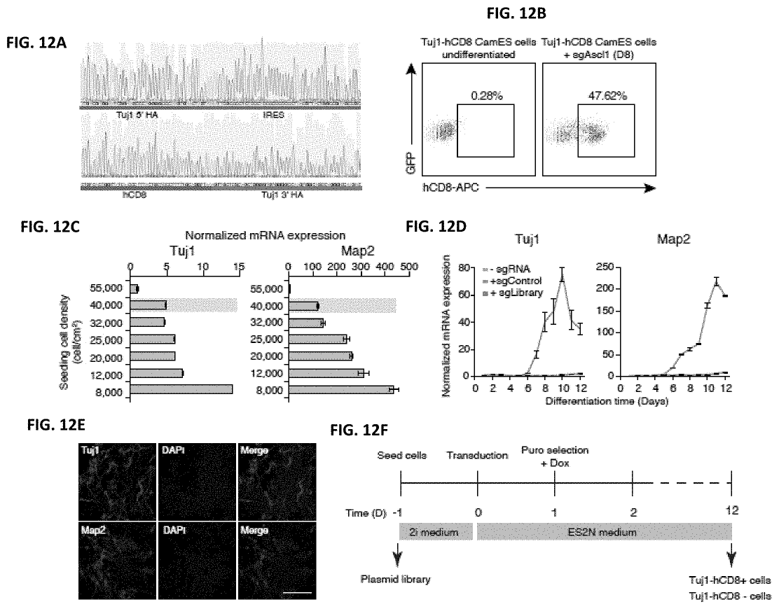

To address these challenges, a clonal reporter CamES (Tuj1-hCD8 CamES) cell line carrying a biallelic human CD8 (hCD8) gene cassette appended downstream to endogenous Tuj1 via an IRES (internal ribosome entry site) was established (FIGS. 5A and 12A). Upon transduction with sgAsc11 and Dox induction, differentiated Tuj1-hCD8 CamES cells expressed both Tuj1 and hCD8 (Figure S5B). MACS (magnetic-activated cell sorting) was used to isolate hCD8+ and hCD8− cells, and observed hCD8+ cells expressed a higher level of neural markers (Tuj1 and Map2) compared to hCD8− cells and unsorted cells (FIG. 5B). This demonstrates that sorted hCD8+ cells are positively correlated with differentiated neuron cells.

The parameters of cell density and differentiation time for screening, which affected neural differentiation efficiency, were determined. 40,000 cells/cm² was chosen as the seeding density, as Tuj1-hCD8 CamES cells transduced the sgRNA library maximized the seeding cell number and showed detectable neural marker expression Tuj1 and Map2 (FIG. 12C). Day 12 was chosen as the sample collection time point, when differentiated cells showed neuronal morphology and expression of neural markers (FIGS. 12D and 12E). With these conditions, MACS was performed to sort and isolate Tuj1-hCD8 positive and negative populations (FIGS. 5A and 12F).

Deep sequencing was used to identify sgRNAs for transcription factors that enhance neural differentiation. The overall distributions of sgRNA from samples collected from plasmid library, sorted Tuj1-hCD8+ and Tuj1-hCD8− cells was compared (FIGS. 5A and 12F). In contrast to the self-renewal screen, a larger fraction of sgRNAs were detected after sorting compared to the plasmid library (FIGS. 5C and 5D). In addition, Tuj1-hCD8+ and Tuj1-hCD8− cells exhibited similar sgRNA depletion.

Stem cell differentiation is affected by stochastic factors. In these experiments, activation of Asc11, a powerful neural inducer, led to only 47.6% of cells being Tuj1-hCD8+(FIG. 12B). In addition, the effects of spontaneous differentiation and less proliferative capacity of desired differentiated cells may affect the overall screening outcome. Thus, most sgRNAs in the non-dropout neural differentiation screen cannot be depleted as strongly as in the dropout screen (FIGS. 5C and 5D). It was contemplated that normalizing positive population against the negative population would more accurately identify the TFs that drive neural differentiation. Thus, paired comparative analysis of Tuj1-hCD8+ and Tuj1-hCD8-cell populations was used to rank the most enriched genes and their sgRNAs.

Validation of Top Enriched sgRNAs Promoting Neural Differentiation

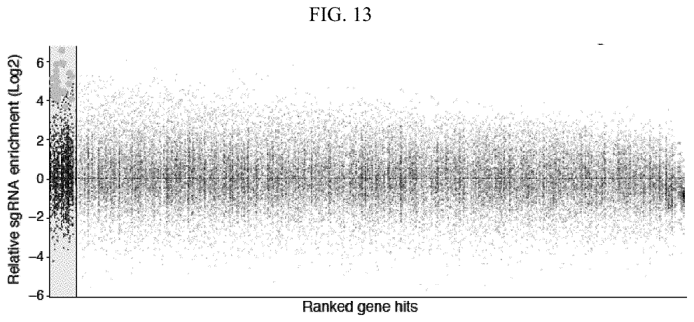

Among the ranked gene hits, the top 20 most effective sgRNAs were chosen for validation (FIGS. 13 and 6A). The 20-gene list contained known neuron-driving transcription factors (Neurog1, Brn2, and Klf12) (Theodorou et al., 2009 Genes Dev. 23, 575-588), and genes that were not previously linked to neural early development including epigenetic regulators (Ezh2, Suz12) and signaling proteins (Jun).

Twenty individual sgRNAs for the top gene hits, as well as 6 non-targeting negative control sgRNAs were tested, Quantitative PCR results showed activation (10 to 10,000 fold) of 19 genes out of 20 tested by their cognate sgRNA (FIG. 6B). Using Tuj1-hCDg CamES cells, Tuj1-hCD8 expression was measured after 12 days of differentiation in basal medium by FACS. All 20 sgRNAs transduced-cells showed expression of hCD8 in a significant percentage of cells (10-50%), while all 6 negative control sgRNAs or cells without a transduced sgRNA showed no hCD8+ cells (FIG. 6C).

Another neuronal marker, NCAM, was used to test differentiation of CamES cells. Similarly, all 20 sgRNAs generated NCAM+ cells (20-60%) after 12 days of differentiation in basal medium, and all negative control sgRNAs showed much less NCAM+ cells (below 10%) (FIG. 6D). Positive immunostaining of neural marker Map2 in all 20 sgRNAs differentiated cells was observed (FIG. 6E). One sgRNA targeting Arnt failed to activate target expression at the time it was assayed for activation. However, this sgRNA was able to induce neural differentiation, which may be due to a longer latency of activation, activation of nearby regulatory elements (e.g., a cis-acting lincRNA), or off-target effects.

Activation of different endogenous genes induced different neural subtypes (FIG. 6F). Most genes induced a high percentage cells expressing neuron markers (Tuj14+, Map2+, and NeuN+). Some hits such as Nr2f1, Nr3c1, and Tcf15 induced more cells with a positive astrocyte marker GFAP. The oligodendrocyte marker Olig2 and the Glutamatergic neuron marker vGluT1 were assayed, and varying levels of expression across the top 20 sgRNAs was observed.

Functional Test and Transcriptome Profiling Confirmed sgJun-Induced Neural Differentiation

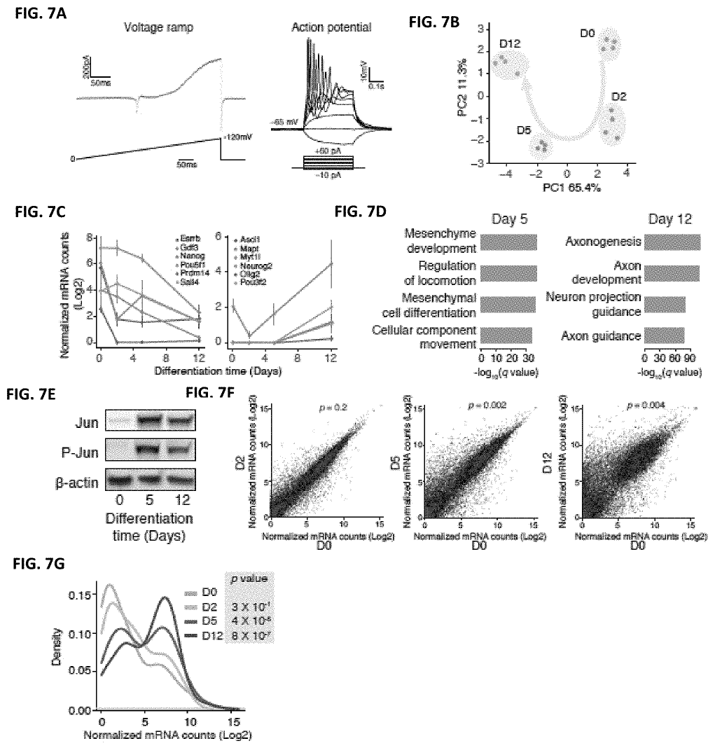

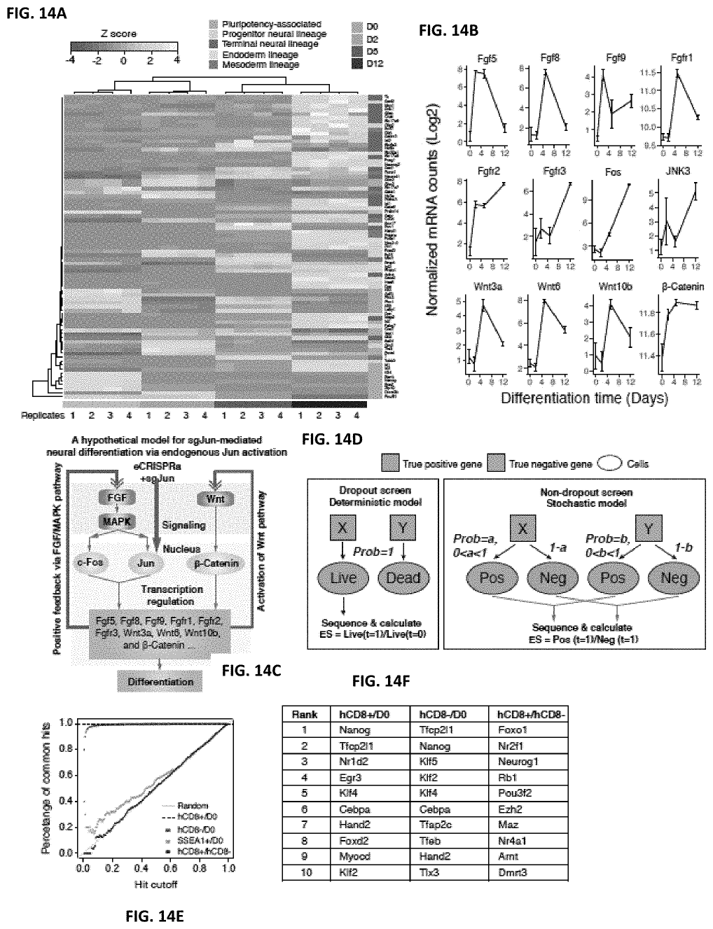

The role of Jun for promoting neural differentiation was examined. Jun has not previously been tied to early neural development. It was observed that sgJun could induce functional neurons that were able to generate action potentials upon current injection (FIG. 7A). RNA-seq was performed to profile the transcriptome of CamES +sgJun cells at various time points (day 0, 2, 5, and 12) (FIG. 14A). Cells were analyzed at different time points using PCA (Principal component analysis), and four distinct clusters that correlated with a dynamic process of neural differentiation were identified (FIG. 7B). It was found that the pluripotency genes were consistently downregulated starting at day 2 after sgJun transduction, and neural marker genes were upregulated throughout the process (FIG. 7C). Meanwhile, day 12 cells were highly enriched for Gene Ontology (GO) terms associated with neural fate and functions, such as axonogenesis and neuron projection guidance (FIG. 7D).

Jun regulates downstream target genes through its phosphorylation and the AP-1 complex formation with c-Fos (Rauscher et al., 1988 Genes Dev. 2, 1687-1699). It was confirmed that endogenous Jun induced by sgJun also was phosphorylated (FIG. 7E). Analysis of AP-1 target genes showed that they were activated at days 5 and 12 (FIG. 7F). It was also found that expression of both FGF ligands and receptors (Fgf5, Fgf8, Egf9, Fgfr1, Fgfr2, and Fgfr3) were rapidly increased at day 2 (FIG. 14B). Meanwhile, key genes of the Wnt pathway (Wnt3a, Wnt6, Wnt10b, and β-catenin) were also upregulated in sgJun-induced cells at days 5 and 12 (FIGS. 7G and 14B).

Previous work reported that overexpression of β-catenin in mouse ES cells induce neurogenesis (Otero et al., 2004: Development 131, 3545-3557). The excessive expression of Wnt genes in the cells indicates that the Wnt pathway plays an important role in sgJun-induced neurogenesis (FIG. 14C). Furthermore, since MAPK, the downstream pathway of FGF, activates Jun via phosphorylation, sgJun-activated endogenous Jun likely maintains its stable expression and sustained activity via a FGF/MAPK positive feedback loop (FIG. 14C), which is consistent with works showing the important role of FGF/MAPK pathway in neural fate commitment of ES cells (Chen et al., 2010 Journal of Biomedical Science 17, 1-11; Ying et al., 2003b Nat. Biotechnol. 21, 183-186). Together, modulation of these pathways through endogenous Jun activation indicates a functional role of Jun for induced neural differentiation of mouse ES cells.

Paired-Analysis is Useful in the Non-Dropout Cell Differentiation Screen

In dropout screens, cells that are negative for the phenotype of interest are almost completely removed from the selected population. Therefore, one can calculate enrichment of the selected population relative to initial pool of sgRNAs to infer functional genes (FIG. 14D). In non-dropout screens, the phenotype of interest may arise stochastically (FIG. 14D). If activation of a gene confers a proliferative advantage, then even if the probability of the phenotype of interest is small (spontaneous differentiation), with more cells it would appear that the gene is enriched in the selected population when compared to the initial population. In fact, a high correlation of enriched genes between the positive and negative Tuj1-hCD8 populations was found (FIG. 14E). The top hits relative to initial sgRNA pool in both populations contain many proliferative genes, but few are related to neural phenotype (FIG. 14F). Those proliferative genes disappear, and several known neural genes are identified when the Tuj1-hCD8+ population was normalized against Tuj1-hCD8− population. The final rankings show little correlation with the enrichment in the positive population (FIGS. 14E and 14F), indicating that these proliferative genes were mostly false positives.

TABLE 1
Primers used to construct individual sgRNAs.

| Primers | sgRNA sequence | SEQ ID NO |
|---|---|---|
| Forward | gtatcccttggagaaccaccttgttgnnnn | 386 |
| primer | nnnnnnnnnnnnnnnngtttaagagctaag | |
| | ctggaaacagca | |
| Reverse | gatcctagtactcgagaaaaaaagcaccga | 387 |
| primer | ctcggtgccac | |
| sgBrn2-1 | gggagagagcttgagagcgc | 388 |
| sgBrn2-2 | gcccaggcgcgtgccgctgcgag | 389 |
| sgBrn2-3 | gcggtatccacgtaaatcaaa | 390 |
| sgBrn2-4 | gctccggtctgggaggttgctag | 391 |
| sgBrn2-5 | gcaccaatcactggctccggtc | 392 |
| sgBrn2-6 | gactgagaagactgggcgcccg | 393 |
| sgBrn2-7 | gaatctgaatcgctgagcta | 394 |
| sgBrn2-8 | gaggccggggacagaagaga | 395 |
| sgBrn2-9 | gagcgcctggaccgaccgcc | 396 |
| sgBrn2-10 | gaaatcgtagtcctgctggctgact | 397 |
| sgBrn2-11 | gtgtgtgtgttcctaggagaa | 398 |
| sgBrn2-12 | gtctagctttggctctcgttct | 399 |
| sgAscl1-1 | ggctgggtgtcccattgaaa | 400 |
| sgAscl1-2 | gaatggagagtttgcaaggag | 401 |
| sgAscl1-3 | gtctggagggaaaagtgtctt | 402 |
| sgAscl1-4 | gagttactgcggagagaagaaa | 403 |
| sgAscl1-5 | gagggaaaggctgctcagaca | 404 |
| Neurog1 | ggctgctgggagttgtgcaa | 405 |
| Myod1 | ggtctccagagtggagtccg | 406 |
| Nanog | ggaagtttcaggtcaagtgg | 407 |
| Mlxip | ggcactccacgtggtgggta | 408 |
| Sox2 | gcctttgcaccctttggatg | 409 |
| Klf2 | gagggtaatagagagaggga | 410 |
| Etv2 | gttcgtggctcacctctggc | 411 |
| Klf4 | gtgcgtatgcgagagagggc | 412 |
| Zc3h11a | gcattatcccttagatgcca | 413 |
| Hsf2 | ggattcgcatggaaagggtt | 414 |
| Hey2 | ggtgtgtctagacaggagac | 415 |
| ZFP36 | ggttgtgtacgaccaactgg | 416 |
| Isl2 | gagaggagaaaggagagggt | 417 |
| Tfeb | gacatgggcaataacagggt | 418 |
| Nobox | gcctgcttgatggaaaggta | 419 |
| Figla | ggcatctgaaaccaggagga | 420 |
| Bcl6 | ggtgggaagagagagagaga | 421 |
| Id1 | ggctcaagaactgaaagggt | 422 |
| Hoxc11 | ggaggagagagagagagggt | 423 |
| M17Rik | gctgataaggtagaaaggta | 424 |
| Foxo1 | ggttcaggatgagtggaggc | 425 |
| Nr2f1 | ggagccaagagaagggctgc | 426 |
| Rb1 | ggctacatacagtctaggtt | 427 |
| Pou3f2 | gaggaaggactgagaagact | 428 |
| Ezh2 | ggttcctttcggcaccttgg | 429 |
| Maz | ggaaggcatctctgggaagc | 430 |
| Nr4a1 | gctaacgtgtagtctcgttg | 431 |
| Arnt | gtttgaaactccaggttaat | 432 |
| Dmrt3 | gaggagttgatagttgttcc | 433 |
| Sin3b | gtgcaagaattcagtccaca | 434 |
| Jun | gagaataaagtgttgtgccg | 435 |
| Suz12 | gaagctctcaaggcgagaaa | 436 |
| Klf12 | gatttgaccatctcttgccg | 437 |
| Nr3c1 | gtcactgctctttaccaaga | 438 |
| Tcf15 | gggatatgctcactttggga | 439 |
| Zeb1 | gaaggaactaagtttcttct | 440 |
| Nr6a1 | gatgacggtcggccgtagtt | 441 |
| Mecom | gattctcaggcagggctcta | 442 |
| Hoxc8 | gctctttcctctaacagccc | 443 |

TABLE 2
Primers used for quantitative PCR.

| | | SEQ |
|---|---|---|
| Gene name | Primer sequence | ID NO |
| RiboL7 F | accgcactgagattcggatg | 444 |
| RiboL7 R | gaaccttacgaacctttgggc | 445 |
| Ascl1 F | aagaagatgagcaaggtggagacg | 446 |
| Ascl1 R | gagatggtgggcgacagga | 447 |
| Brn2 F | tttcctcaaatgccctaagc | 448 |
| Brn2 R | ggaggggtcatccttttctc | 449 |
| Tuj1 F | agtcagcatgagggagatcg | 450 |
| Tuj1 R | agtcccctacatagttgccg | 451 |
| Map2 F | agcactgattgggaagcact | 452 |
| Map2 R | caattcaaggaagttgtaaagtagtgaag | 453 |
| | tttg | |
| Nanog F | aaccaaaggatgaagtgcaagcgg | 454 |
| Nanog R | tccaagttgggttggtccaagtct | 455 |
| Mlxip F | aagctcttcgagtgcatgac | 456 |
| Mlxip R | ttgttgagccggatcttgtc | 457 |
| Sox2 F | acaagagaattgggaggggt | 458 |
| Sox2 F | ttttctagtcggcatcaccg | 459 |
| Klf2 F | ccttcggtcttttcgagga | 460 |
| Klf2 R | cttggcctccagcagctc | 461 |
| Etv2 F | acgtagaaggctgctggaa | 462 |
| Etv2 R | tgtccagtctcgcgacca | 463 |
| Klf4 F | aaaagaacagccacccacac | 464 |
| Klf4 R | cgtcccagtcacagtggtaa | 465 |
| Zc3h11a F | catcggttcggtaaagtttctgt | 466 |
| Zc3h11a R | ccactcagccacagaaatcg | 467 |
| Hsf2 F | tgaagcagagttccaacgtg | 468 |
| Hsf2 R | ttgctcatccaagaccagaa | 469 |
| Hey2 F | tgaagatgctccaggctaca | 470 |
| Hey2 F | tctgtcaagcactctcggaa | 471 |
| Zfp36 F | tctcttcaccaaggccattc | 472 |
| Zfp36 R | tatgttccaaagtcctccga | 473 |
| Isl2 F | agtcgaggtgcagacgtac | 474 |
| Isl2 R | ttgcctagggagcctgact | 475 |
| Tfeb F | caacagtgctcccaacagtc | 476 |
| Tfeb R | ttgatgtagcccagcacgc | 477 |
| Nobox F | acggagaagctctgcaagaa | 478 |
| Nobox R | ttgtcttgatcatcctggatgg | 479 |
| Figla F | actcggctgtgttctggaag | 480 |
| Figla R | tgggtagcatttcccaagag | 481 |
| Bcl6 F | ttggactgtgaagcaaggca | 482 |
| Bcl6 R | actccggaggcgattaagg | 483 |
| Id1 F | ctgaacggcgagatcagtg | 484 |
| Id1 R | tttcctcttgcctcctgaag | 485 |
| Hoxc11 F | aacacgaatcccagctcgt | 486 |
| Hoxc11 R | ggatctggaatttcgaataagggc | 487 |
| M17Rik F | cctgagactaatactgtatgatttggaaa | 488 |
| M17Rik R | cacaggtttagagataaccaaagtgg | 489 |
| Foxo1 F | gagtggatggtgaagagcgt | 490 |
| Foxo1 R | tgctgtgaagggacagattg | 491 |
| Nr2f1 F | ccaacaggaactgtcccatc | 492 |
| Nr2f1 R | attcttcctcgctgaaccg | 493 |
| Neurog1 F | cggcttcagaagacttcacc | 494 |
| Neurog1 R | ggcctagtggtatgggatga | 495 |
| Rb1 F | gcagcatcttgattctggaac | 496 |
| Rb1 R | tgtcaagttggcttccacttt | 497 |
| Pou3f2 F | tttcctcaaatgccctaagc | 498 |
| Pou3f2 R | ggaggggtcatccttttctc | 499 |
| Ezh2 F | acttctgtgagctcattgcg | 500 |
| Ezh2 R | cgactgcattcagggtcttt | 501 |
| Maz F | gtggcaagatgctgagctc | 502 |
| Maz R | cattggacaaacctcaccagtac | 503 |
| Nr4a1 F | gctagaaggactgcggagc | 504 |
| Nr4a1 R | attgagcttgaatacagggca | 505 |
| Arnt F | ggcgactacagctaacccag | 506 |
| Arnt R | gccctctgtacaacagctcc | 507 |
| Dmrt3 F | agcgcagcttgctaaacc | 508 |
| Dmrt3 R | gcttttgacaacatctgggg | 509 |
| Sin3b F | agagttcggacagttcctgc | 510 |
| Sin3b R | tcctcattcttctgcccact | 511 |
| Jun F | gaaaagtagcccccaacctc | 512 |
| Jun R | aatcagacaggggacacagc | 513 |
| Suz12 F | tcgaaattccagaacaagca | 514 |
| Suz12 R | tgtggaagaaaccggtaaatg | 515 |
| Klf12 F | ccataaagaatctcagcgcc | 516 |
| Klf12 R | ccatatcggggtagttgtgg | 517 |
| Nr3c1 F | ggacaacctgacttccttgg | 518 |
| Nr3c1 R | ctggacggaggagaactcac | 519 |
| Tcf15 F | tctgcaccttctgtctcagc | 520 |
| Tcf15 R | aaccagggatccaggttcat | 521 |
| Zeb1 F | acagagaatggaatgtatgcatgtg | 522 |
| Zeb1 R | agattccacactcgtgaggc | 523 |
| Nr6a1 F | gcaacggtttctgtcaggat | 524 |
| Nr6a1 R | ggttcgttgttcagctcgat | 525 |
| Mecom F | acagcatgagatccaaaggc | 526 |
| Mecom R | ttatcccatctgcatcagca | 527 |
| Hoxc8 F | aaatcctccgccaacactaa | 528 |
| Hoxc8 R | tgtaagtttgtcgaccgctg | 529 |

TABLE 3
Top 100 gene hits from CRISPRa self-renewal screen.

| Rank | Gene name | Enrichment score |
|---|---|---|
| 1 | Nanog | 6.436538099 |
| 2 | Sox2 | 5.110480488 |
| 3 | Klf4 | 4.679609611 |
| 4 | Bc16 | 4.250485879 |
| 5 | Tfeb | 4.160094948 |
| 6 | Mlxip | 3.992616854 |
| 7 | Klf2 | 3.911099626 |
| 8 | Etv2 | 3.644806172 |
| 9 | Isl2 | 3.468873873 |
| 10 | Hey2 | 3.189713541 |
| 11 | Zfp36 | 2.929649816 |
| 12 | Zc3h11a | 2.83067826 |
| 13 | Sox18 | 2.813521887 |
| 14 | Nobox | 2.627399442 |
| 15 | Figla | 2.607875769 |
| 16 | 4921504A21Rik | 2.594074135 |
| 17 | Hsf2 | 2.552027639 |
| 18 | Hoxc11 | 2.518020583 |
| 19 | Tfcp211 | 2.460616651 |
| 20 | Spi1 | 2.383834061 |
| 21 | Id1 | 2.277631872 |
| 22 | Tlx2 | 2.237444329 |
| 23 | 4930555M17Rik | 1.890123174 |
| 24 | Nov | 1.874188087 |
| 25 | Klf5 | 1.852149928 |
| 26 | Crygf | 1.829064836 |
| 27 | Sox11 | 1.807058979 |
| 28 | Atf5 | 1.774675631 |
| 29 | Esrrg | 1.746829472 |
| 30 | Tsn | 1.744364085 |
| 31 | Thrb | 1.602037631 |
| 32 | Nfe212 | 1.593264465 |
| 33 | Lhx1 | 1.548863185 |
| 34 | Pou5fl | 1.518892786 |
| 35 | Ebfl | 1.452433199 |
| 36 | Dlx5 | 1.403820123 |
| 37 | Mycl | 1.371065103 |
| 38 | Atfl | 1.36137151 |
| 39 | Tftdp1 | 1.326446848 |
| 40 | Irx6 | 1.194061551 |
| 41 | Zfp2 | 1.191847857 |
| 42 | Nfatc1 | 1.188011066 |
| 43 | Crem | 1.049272101 |
| 44 | Nr3c1 | 1.042323412 |
| 45 | Pax5 | 1.024334324 |
| 46 | Foxfl | 1.00419091 |
| 47 | Snai1 | 0.960150521 |
| 48 | Zfp423 | 0.947760908 |
| 49 | Esrrb | 0.904004441 |
| 50 | Pbx2 | 0.899430618 |
| 51 | Foxd4 | 0.895608808 |
| 52 | Sox1 | 0.878521398 |
| 53 | Lbx1 | 0.841046411 |
| 54 | Mecom | 0.820757135 |
| 55 | Ncor2 | 0.780231219 |
| 56 | Nr0b2 | 0.752404477 |
| 57 | Trp53 | 0.743632306 |
| 58 | Lmo3 | 0.732198452 |
| 59 | En1 | 0.731989985 |
| 60 | Rfx1 | 0.725576385 |
| 61 | Maz | 0.700348134 |
| 62 | Alx4 | 0.686172162 |
| 63 | Nr1d2 | 0.679722158 |
| 64 | Tcf15 | 0.620553011 |
| 65 | Egr3 | 0.617223131 |
| 66 | Nr5a1 | 0.614144289 |
| 67 | Tfe3 | 0.60710143 |
| 68 | Spdef | 0.593267507 |
| 69 | Tcfl2 | 0.564881228 |
| 70 | Dlx2 | 0.541542994 |
| 71 | Vezfl | 0.534712227 |
| 72 | Gata1 | 0.504194994 |
| 73 | Arf6 | 0.491842327 |
| 74 | Sox21 | 0.477561748 |
| 75 | Lmx1a | 0.447377739 |
| 76 | Pou4f2 | 0.410773196 |
| 77 | Nr1b2 | 0.406668822 |
| 78 | Fox11 | 0.394295617 |
| 79 | Stat5b | 0.369982068 |
| 80 | Evx2 | 0.360115239 |
| 81 | Sox5 | 0.348850095 |
| 82 | Hivep3 | 0.324844194 |
| 83 | Tfap2a | 0.303852954 |
| 84 | Glis3 | 0.277004435 |
| 85 | Mafk | 0.265635614 |
| 86 | Hoxb5 | 0.256534563 |
| 87 | Myf5 | 0.252944449 |
| 88 | Nkx2-5 | 0.251102596 |
| 89 | Lhx6 | 0.244502182 |
| 90 | Foxs1 | 0.242003106 |
| 91 | Rnps1 | 0.2417908 |
| 92 | Mitf | 0.229103445 |
| 93 | Drd1a | 0.21477535 |
| 94 | Lmx1b | 0.191984237 |
| 95 | Vax2 | 0.183188363 |
| 96 | Hoxa11 | 0.1661187439 |
| 97 | Otp | 0.163494265 |
| 98 | Mxd4 | 0.160929842 |
| 99 | Plag11 | 0.137545433 |
| 100 | Smad5 | 0.128584689 |

TABLE 4
Top 100 gene hits from CRISPRa neural differentiation screen.

| Rank | Gene name | Enrichment score |
|---|---|---|
| 1 | Foxo1 | 2.49122811 |
| 2 | Nr2fl | 2.448600182 |
| 3 | Neurog1 | 2.43849068 |
| 4 | Rb1 | 2.435300527 |
| 5 | Pou3f2 | 2.385360453 |
| 6 | Ezh2 | 2.380072461 |
| 7 | Maz | 2.361103604 |
| 8 | Nr4a1 | 2.351837703 |
| 9 | Arnt | 2.317336958 |
| 10 | Dmrt3 | 2.304207908 |
| 11 | Sin3b | 2.280599668 |
| 12 | Jun | 2.277732884 |
| 13 | Suz12 | 2.276236754 |
| 14 | KIfl2 | 2.269476929 |
| 15 | Nr3cl | 2.249983644 |
| 16 | Tcfl5 | 2.229200027 |
| 17 | Zeb1 | 2.221200461 |
| 18 | Nr6a1 | 2.208496165 |
| 19 | Mecom | 2.207944981 |
| 20 | Trim24 | 2.206262504 |
| 21 | Hoxc8 | 2.184103377 |
| 22 | Foxk1 | 2.171388615 |
| 23 | 2410080102RiK | 2.171161939 |
| 24 | Nr4a3 | 2.168779599 |
| 25 | Trp73 | 2.16579857 |
| 26 | Foxs1 | 2.162897697 |
| 27 | Ikzf3 | 2.15938851 |
| 28 | Nkx2-6 | 2.15063949 |
| 29 | Sox11 | 2.140964961 |
| 30 | 1110054M08Rik | 2.139005342 |
| 31 | Crem | 2.133968618 |
| 32 | Meis3 | 2.131453549 |
| 33 | Bmyc | 2.130409666 |
| 34 | Epas1 | 2.129339686 |
| 35 | Nr2f6 | 2.128397081 |
| 36 | Nacc1 | 2.120269011 |
| 37 | Bsx | 2.120136772 |
| 38 | Foxd3 | 2.114601186 |
| 39 | Myog | 2.107435864 |
| 40 | Smad3 | 2.105254748 |
| 41 | Wt1 | 2.091731056 |
| 42 | Taz | 2.091306567 |
| 43 | Smad7 | 2.071136269 |
| 44 | Stra13 | 2.06971649 |
| 45 | Hoxc4 | 2.062634453 |
| 46 | Pou3f3 | 2.058607569 |
| 47 | Zbtb12 | 2.051837502 |
| 48 | Atf5 | 2.042025795 |
| 49 | Gtf2a2 | 2.041587014 |
| 50 | Pura | 2.040735147 |
| 51 | Snai1 | 2.040229657 |
| 52 | Ncor1 | 2.038396405 |
| 53 | Pcbp2 | 2.036271048 |
| 54 | E2f2 | 2.028758908 |
| 55 | Nfkbib | 2.023153101 |
| 56 | Gli2 | 2.021010016 |
| 57 | Nr0b1 | 2.020715359 |
| 58 | B230110C06Rik | 2.016733057 |
| 59 | T | 2.014396786 |
| 60 | Runx3 | 2.011724145 |
| 61 | Rxra | 2.011600497 |
| 62 | Mafk | 2.009964981 |
| 63 | Foxnl | 2.006315586 |
| 64 | Smad4 | 1.999197443 |
| 65 | Meis2 | 1.998728368 |
| 66 | Hoxa1 | 1.996287157 |
| 67 | Zic1 | 1.992579239 |
| 68 | Sebox | 1.99248237 |
| 69 | Nfyc | 1.983084664 |
| 70 | Lmx1b | 1.980716237 |
| 71 | Lhx3 | 1.979175342 |
| 72 | Hmx2 | 1.978886945 |
| 73 | Arf6 | 1.977331424 |
| 74 | Nfatc3 | 1.975872129 |
| 75 | Neurod6 | 1.973516686 |
| 76 | Smarca4 | 1.972359038 |
| 77 | Twist1 | 1.971479015 |
| 78 | Gzfl | 1.963483117 |
| 79 | Hoxcl0 | 1.962998475 |
| 80 | Tbx4 | 1.962626034 |
| 81 | Npas2 | 1.962608209 |
| 82 | Ctbp1 | 1.960624385 |
| 83 | Gcm2 | 1.960206991 |
| 84 | Is12 | 1.957324105 |
| 85 | Arid5a | 1.956887379 |
| 86 | Lef1 | 1.955552772 |
| 87 | RP24-399L6.2 | 1.953337042 |
| 88 | Smad5 | 1.949029539 |
| 89 | Lbx1 | 1.948838891 |
| 90 | Pax3 | 1.945680745 |
| 91 | Foxj1 | 1.944149198 |
| 92 | Tbx5 | 1.943975816 |
| 93 | Barh11 | 1.943598679 |
| 94 | Hoxd11 | 1.9410811 |
| 95 | Pou1fl | 1.939557398 |
| 96 | Klf3 | 1.938997548 |
| 97 | Pcbp1 | 1.937292841 |
| 98 | Evx2 | 1.935442174 |
| 99 | Irx5 | 1.934100096 |
| 100 | Nkx6-3 | 1.928635054 |

### Example 2

Quantitative Genetic Interaction Mapping Using CRISPRI

### A. Methods

The vectors used in this study were constructed by using standard molecular cloning techniques, including PCR, restriction enzyme digestion and ligation. Custom oligonucleotides were from Integrated DNA Technologies. E. coli strain D1H5a was used for the transformation and selected by 100 μg/ml of carbenicillin, or 50 μg/ml of Kanamycin. DNA was extracted and purified using Plasmid Mini or Midi Kits (Macherey-Nagel). Sequences of the vector constructs were verified with Quintarabio's DNA sequencing service.

Construct Design

The dCas9-KRAB plasmid and sgRNA expressing plasmid are previously described vectors (Du, D. & Qi, L S. Cold Spring Harbor Protocols 2016, (2016)). The SpeI and Sail sites were mutated in the sgRNA expression plasmid. The single sgRNA expression plasmids were cloned as described previously with minor modifications. Briefly, the plasmids were cloned by PCR from an existing sgRNA template using a unique 50 primer containing the desired protospacer (N is the protospacer) and a common primer with (SpeI and SalI sites). The PCR products and the lentiviral mice 16 (mU6) based sgRNA expression vector were digested with BstXI and XhoI and the two pieces of DNA were ligated together. The single vector was introduced unique SpeI and SalI sites to enable the insertion of the mU6-sgRNA expression cassettes.

To construct a lentiviral vector for mU6-driven expression of combinatorial gRNAs, mU6-sgRNA expression cassettes were prepared from digestion of the storage vector with XbaI and XhoI enzymes, and inserted into the target single sgRNA expression vector backbone, using ligation via the compatible sticky ends generated by digestion of the target single sgRNA expression vector with SpeI and SalI enzymes.

The Single Library Cloning

A library of 336 sgRNAs targeting a set of 112 genes encoding epigenetic regulators (3 sgRNAs/gene) was constructed using top prediction hits from the CRISPR-ERA algorithm (Liu, H, et al Bioinformatics 31, 3676-3678 (2015)). The library also included 30 non-targeting negative control sgRNAs. sgRNAs containing XbaI, XhoI, SpeI, and SalI restriction sites, which were used for double sgRNA library construction, were excluded. Individual oligos encoding sgRNAs were synthesized in a 384-well format, pooled, and the single sgRNA expression vectors were constructed individually by ligating the oligos into a common sgRNA lentiviral vector with SpeI and SalI sites. After sequencing validation, 336 sgRNA constructs were manually mixed with equal amount for the single sgRNA screens and double sgRNA library construction. The sgRNA sequence and corresponding genes are listed in Table 5.

Combinatorial sgRNA Library Pool

To generate the pooled storage vector library, the 336 single sgRNA expression vectors were mixed equally. Pooled lentiviral vector libraries harboring combinatorial gRNA(s) were constructed with the same strategy as for the generation of combinatorial sgRNA constructs described above, except that the assembly was performed with pooled inserts and vectors, instead of individual ones. Briefly, the pooled mU6-sgRNA inserts were generated by a single-pot digestion of the pooled storage vector library with XbaI and XhoI. The destination lentiviral vectors were digested with SpeI and SalI. The digested inserts and vectors were ligated via their compatible ends (i.e., XbaI+SalI & XhoI+SpeI) to create the pooled double sgRNA library (336×336=112,896 total combinations) in the lentiviral vector. The lentiviral sgRNA library pools were prepared in DHS ultra-competent cells (Agilent Technologies) and purified by Plasmid Midi Kit (Macherey-Nagel). The sequences of the deep sequencing is listed in Table 6.

Cell Culture

1HEK293T and HEK293 cells were cultured in DMEM supplemented with 10%/6 fetal bovine serum, 100 units/ml streptomycin and 100 mg/ml penicillin at 3TC, with 5% CO₂. To generate inducible CRISPRi HEK293 (TetOn-dCas9-KRAB) cell line, the cells were lentivirally transduced with constructs that express dCas9-KRAB from the TRE3G promoter and rtTA. Pure polyclonal populations of CRISPRi cell line were treated with doxycycline, and sorted by flow cytometry using a BD FACS Aria2 for mCherry expression. These cells were then grown in the absence of doxycycline until mCherry fluorescence reduced to uninduced levels.

Lentivirus Production and Transduction

Lentiviruses were produced and packaged in HEK293T cells as described previously with minor modification (Du et al., 2016, supra). Briefly, HEK 293′T were transfected with standard packaging vectors using Mirus TransIT-LT1 transfection reagent (Mirus MIR 2300) according to the manufacturer's instructions. Viral supernatant was harvested 48-72 h following transfection and either filtered through a 0.45 μm syringe filter or snap-frozen.

Growth Competition Assay

Cells were grown at minimum library coverage of 1,000 for the screens. The target cells were infected in the presence of 8 μg/ml polybrene (Sigma) at a multiplicity of infection of about 0.3 to ensure single copy integration in most cells, which is corresponded to an infection efficiency of 30-40%. For single library screens, cells were grown in the flasks and harvested at 0, 12 and 20 days after puromycin selection; for double library screens, cells were grown in the flasks and harvested at 0, 8 and 16 days after puromycin selection. Cells were maintained at least 1,000 cells per sgRNA for each screen.

After the cell samples were collected, the genomic DNA was isolated using QIAamp DNA Blood Maxi Kit (Qiagen) according to the manufacturer's protocol, the cassette encoding the sgRNA was amplified by PCR, and relative sgRNA abundance was determined by next generation sequencing on an Illumina Miseq for single screens or an lllumina HiSeq-2500 for double screens using custom primers with previously described protocols at high coverage (Bassik, M. C. et al. Cell 152, 909-922(2013); Roguev, A. et al. Nat. Methods 10, 432-437 (2013)). Two biological replicates of each screen were performed.

For the cell growth validation experiments, the viruses with single sgRNAs or double sgRNA were transduced into HEK293 (TetOn-dCas9-KRAB) cells, followed by the selection with 2 μg/ml puromycin to remove the uninfected cells. Three days after the cells were treated with or without Dox (0.5 ug/ml), the cell viability was measured by XTT assay (Biotium) according to the manufacturer's experimental protocol. 2,000 to 10,000 cells were plated into 96-well tissue culture plates for the growth assay. For each 96 well, 30 μl of XTT solution was added to the 100 ul cell cultures at the time points indicated. Cells were incubated for 6 hours at 37 C with 5% CO₂. Measure the absorbance signal of the samples with a spectrophotometer at a wavelength of 450-500 nm. Measure background absorbance at a wavelength between 630-690 nm. The normalized absorbance values were obtained by subtracting background absorbance from signal absorbance.

Validation of Gene Hits

Cells were harvested and total RNA was isolated using the RNAeasy Kit (Qiagen), according to manufacturer's instructions. RNA was converted to cDNA using iScript™ cDNA Synthesis Kit according to manufacturer's instructions (Bio-rad). Quantitative PCR reactions were prepared with a 2× master mix according to the manufacturer's instructions (Bio-rad). Reactions were run on CFX96 Touch™ Real-Time PCR Detection System (Bio-rad). Primer sequences for qPCR are listed in Table 3.

### Results

To develop a CRISPRi combinatorial screening approach, a single library consisting of 336 sgRNAs using was constructed using a computational algorithm (Liu, H. et al. Bioinformatics 31, 3676-3678 (2015)), which sequence-specifically targeted 112 genes (3 sgRNA/gene) involved in chromatin regulation (for the gene list and their sgRNAs, see Table 5). The library also included 30 negative control sgRNAs without target sites in the human genome. Pooled cloning of 336 sgRNAs onto itself generated a mixed double sgRNA library containing 112,896 (336×336) combinations. Both libraries were prepared as lentivirus pools ready for large-scale mammalian cell transduction at a low multiplicity of infection (MOI=0.3).

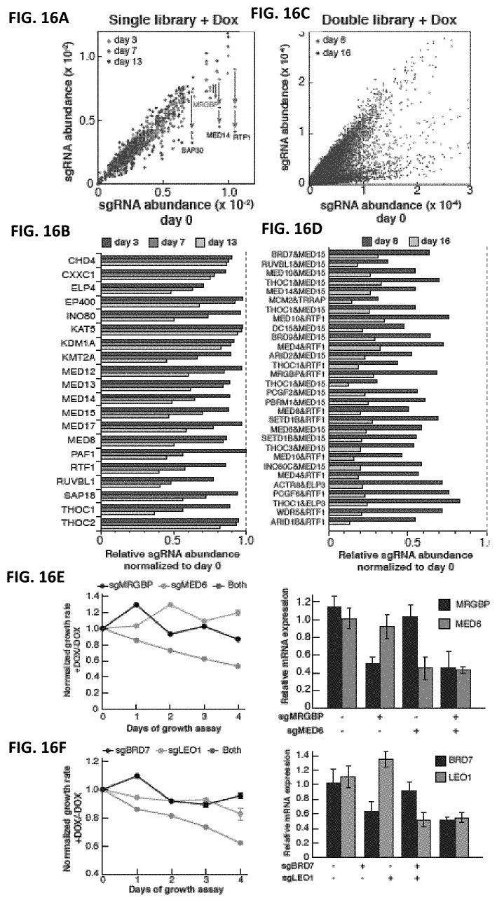

The repressive dCas9-KRAB protein was conditionally expressed under the control of the Doxycycline (Dox)-inducible promoter TetON-3G in the human embryonic kidney 293 (HEK293) cells. Transducing both libraries into clonal HEK293-dCas9-KRAB cells generated two pooled cell populations (FIG. 16A): one with 336 single perturbations and the other with 112,896 double perturbations. Adding Dox to cells could induce expression of dCas9-KRAB to repress target gene(s) guided by co-expressed sgRNA(s) and monitored the growth phenotype from single or double gene perturbations. Pair-ended deep sequencing of sgRNA library distribution for each library (Mi-seq for single library and Hi-seq for double library) was performed with and without Dox, as well as at different time points.

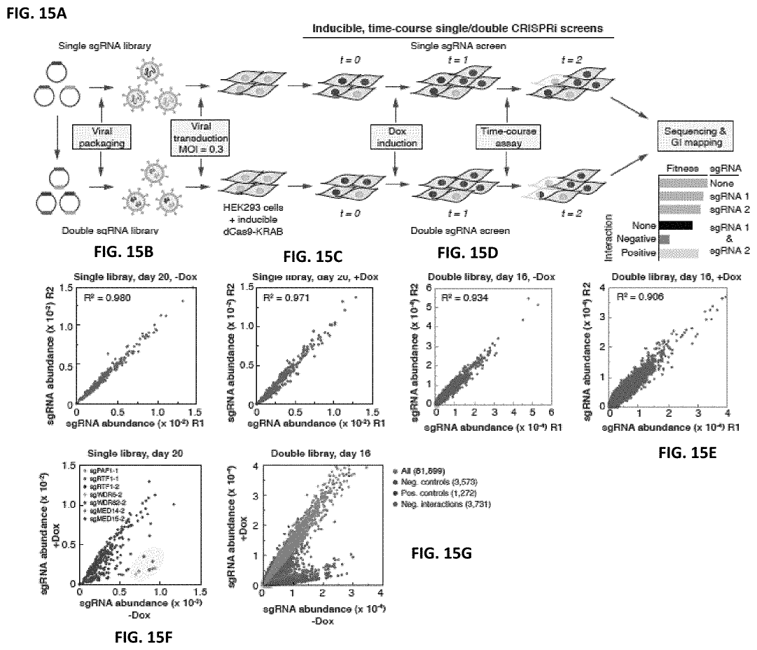

It was first investigated if sgRNA distribution remained consistent between biological replicates before and after library screening. Sequencing single and double libraries with or without Dox at different time points exhibited consistently high coefficient of determination (R²) (FIG. 15B-E). For example, R² was 0.980 without Dox induction and 0.971 with Dox for the single library (day 20) (FIG. 15B-C); and for the double library (day 16), 0.934 without Dox and 0.906 with Dox (FIG. 15D-E). sgRNA distribution from biological replicates was assayed at other time points and similarly high correlation was observed. Together these data demonstrate that the experimental platform produces data of very high reproducibility.

It was next determined if inducible expression of dCas9-KRAB allowed one to identify single and double gene perturbations that influenced cell growth (FIGS. 15F & G). It was observed that repression of a set of individual genes dramatically slowed down cell growth in the presence of Dox compared to without (FIG. 15F). This list of genes included gene components of the mediator complex (MED14 and MED15), components of the histone H3-Lys4 methyltransferase complex (WDR82 and WDR5), and RNA polymerase II associated factors (PAF1 and RTF1). Double library culture showed a large number of combinatorial perturbations significantly reduced cell growth with Dox, with an overall bifurcation pattern, wherein the negative controls fell along the diagonal line and the positive controls were biased from the diagonal line (FIG. 15G).

The above inducible experiments were performed at end time points. sgRNA distribution was further compared for both single and double libraries with and without Dox induction at intermediate time points (day 12 for single library and day 8 for double library). Consistent phenotypes at these time points compared to end time points were observed. For example, a similar list of genes whose repression slowed down cell growth, including ME14, MED15, WDR82, PAF1, and RIF1. The absence of WDR5 at day 12 indicates that WDR5 has a moderate role for growth compared to other gene hits. For the double library, a similar bifurcation pattern was observed, with a difference that the bifurcation degree (measured by the angle between the two populations) is smaller at earlier time points.

The consistent gene hits and dropout pattern for both libraries between different time points propelled a comparison of datasets across a broad time course. It was investigated if the trend of dropout effects could provide another layer of identification of true positive hits (FIG. 16). For the single library in the presence of Dox, sgRNA enrichment was compared at days 0, 3, 7, and 13 (FIG. 16A). While some genes showed consistent depletion (e.g., RTF1, MED14, SAP30), many other genes showed inconsistent enrichment (e.g., MRGBP). Among 112 epigenetic factors, 20 genes were observed to exhibit consistent depletion over time, showing inhibition of these genes constantly slowed down cell growth (FIG. 168). The double library similarly showed temporal dropout of pairwise sgRNAs assayed at days 0, 8 and 16 (FIG. 16C). Over time, a large number of combinations were consistently depleted as a selection of these was plotted as in FIG. 16D.

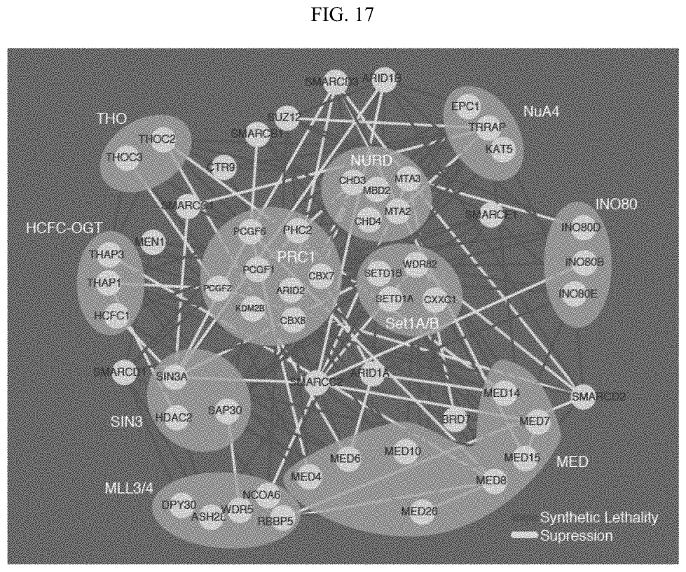

The time-course sgRNA enrichment was compared in the absence of Dox for both single and double libraries. No significant changes of sgRNA distribution were observed over time for both libraries without Dox. For the single library, comparing the day 0 sample with day 12 or day 20 samples (+/− Dox) showed only dropout of gene hits with Dox (FIGS. 17A-B), and for the double library comparing day 0 with day 8 or day 16 (+/− Dox) confirmed similar conclusions. Altogether, these experiments confirmed that the system enables inducible, temporal screens of genetic interactions.

Two negative interactions were validated, demonstrating their ability to suppress cell proliferation and causing repression of target endogenous genes. Two pairs were chosen for testing: MGBRP/MED6, and BRD7/LEO1. MRGBP is a component of the NuA4 histone acetyltransferase complex involved in gene activation by acetylation of histones; BRD7 is a member of the bromodomain-containing protein family; and LEO1 is a component of the PAF1 complex (PAF1C) involved in transcription of RNA Pol II. The results confirmed the validity of the double repression and synthetic lethality-based growth effects. As shown in FIGS. 16E & 16F, repression of two genes simultaneously (MGBRP & MED6 for FIG. 16E; and BRD7 & LEO1 for FIG. 16F) suppressed cell growth over time, while repression of individual genes did not cause significant growth inhibition. Quantitative PCR results further confirmed the repressive effects of the sgRNAs on the corresponding genes either individually or combinatorically delivered into cells. Notable, the moderate repression effects of the tested sgRNAs supported the strong growth effects of the genetic interacting pairs. Future optimization of CRISPRi repression efficacy allow one to perform screens at different strengths (weak, medium, strong) of gene repression.

Based on the curated set of protein complexes and pathways, a GI map depicting the genetic cross-talk between different functional modules involved in chromatin was created (FIG. 17). Using a scoring system similar to the S-score (Collins, et al., J. Meth. Enzymol. 470, 205-231 (2010)), 68 negative and 47 positive genetic interactions were identified. Contained within this map are modules corresponding to the INO80 chromatin remodeling complex; the mediator complex (MED); the NuA4 histone acetyltransferase (HAT) complexes; the Nucleosome Remodeling Deacetylase NURD complex; the histone methyltransferase (HMT) complex SET1A/B; the Polycomb complex PRC1; the histone 3 lysine-4 methyltransferases MLL3/4; the SIN3 transcription repressor; Host Cell Factor C (HCFC)-glycosyltransferase (OGT) complex; and nuclear THO transcription elongation complex. Notably, the mediator complex occupies a large set of interactions on the map, interacting strongly, both positively and negatively, with many other functional modules. For example, strong positive GIs were observed between the MED complex and modules corresponding to PRC1 and the SET1A/B complex. Furthermore, strong negative interactions were observed between components of the SIN3 complex and many other modules of mediator components and SWI/SNF family of protein SMARCC2.

The nuclease Cas9 for gene editing-mediated knockout allows complete loss of function, yet knockout can be heterogeneous among alleles due to existence of in-frame indels. On the contrary, CRISPRi-based dCas9 transcription knockdown leads to partial, homogeneous loss of function (Mandegar, M. A. et al. Cell Stem Cell 18, 541-553 (2016)). Applying the two methods to higher-order genetic screening needs to consider these important differences. For example, as epistatic genetic screens require simultaneous perturbation of multiple genes (usually 2 genes, 4 alleles), the heterogeneity of gene knockout in pooled CRISPR screens may result in a significant portion of cells without proper epistatic perturbation. Among the cells that are properly perturbed, complete knockout of function offers a highly sensitive way to discover novel gene combinations whose perturbation leads to measurable phenotypes (e.g., growth). Yet, combinatorial multiple gene knockouts may easily cause lethal effects by itself, precluding testing other phenotypes (e.g., differentiation or host-pathogen interaction).

On the contrary, partial knockdown by CRISPRi, while being less sensitive than CRISPR knockout, likely avoids major dominating lethal effects. The homogeneous transcriptional repression could generate cell populations with consistent multi-gene perturbation. Furthermore, sgRNAs binding at various loci along the promoter lead to varying levels of CRISPRi repression, which is contemplated to provide dosage-dependent combinatorial screening distinct from binary perturbation from CRISPR. The demonstration of the inducible and titratable features of CRISPRi combinatorial screening showed the method allows assaying genetic interactions temporally and potentially in a dose-dependent manner.

Compared to RNAi-based methods, the major approach for genetic interaction mapping, CRISPRi presents a few advantages as well. CRISPRi knockdown is specific (Gilbert, L. A. et al., Cell 159, 647-661 (2014)), with less concerns about multiple sgRNAs in the same cells causing unexpected off-target perturbation. As CRISPR activation (CRISPRa) is based on somewhat similar setup as CRISPRi, by changing a repressive effector into an activating effector, the same approach can be expanded into gain-of-function screening of pairwise of genes. Furthermore, combining CRISPRi and CRISPRa into the some cells is contemplated to allow simultaneous activating a gene while repressing another gene. These dramatically expand the modes of epistatic screens that can be performed.

Development of high-throughput epistasis-mapping technologies has made it possible to interrogate complex biological phenomena. Mapping the PPI networks and GI networks have become major methodologies to study epistasis. The PPI networks report on gene products that interact physically; (GIs, in contrast, illustrate functional relationships between genes including, but not limited to, physical interactions of their gene products. They often reveal how groups of proteins and complexes work together to carry out biological functions and can describe the cross-talk between pathways and processes. Therefore, the method for mapping GI networks in mammalian cells provides a useful, natural complement to PPI mapping methods and other existing GI mapping methods. Integrating the two types of information is extremely powerful in understanding complex biology in broader contexts of basic and translational research.

TABLE 5
Gene list and sgRNA sequences

| Gene name | sgRNA sequence | SEQ ID NO |
|---|---|---|
| ACTL6A_1 | GTGGGTGGCGGTGGAAGTTA | 1 |
| ACTL6A_2 | GGCCGCGACTGCGAGTCTCG | 2 |
| ACTL6A_3 | GCGCCGGCAGCAGCCATGAG | 3 |
| ACTR8_1 | GCGCTGCAGCCACGACTGCC | 4 |
| ACTR8_2 | GTCTCCGGCCATAATGACCC | 5 |
| ACTR8_3 | GCGGCCCATCGTGCCCGCGC | 6 |
| ARID1A_1 | GGCTCTGTAGGCTCGGGACC | 7 |
| ARID1A_2 | GGAGAAGACGAAGACAGGGC | 8 |
| ARID1A_3 | GCCCCCCTCATTCCCAGGCA | 9 |
| ARID1B_1 | GCATCCTCTTCCTCCTCGTC | 10 |
| ARID1B_2 | GGGGAGCAGCCCCGTCTCCA | 11 |
| ARID1B_3 | GAGGCGGCTCTCAAGGAGGG | 12 |
| ARID2_1 | GGAACTGCCGCAGCTCGTCC | 13 |
| ARID2_2 | GAACCGGGGGGGCAGCGCCG | 14 |
| ARID2_3 | GGGGTCCCGGCTGACAAGTG | 15 |
| ASH2L_1 | GGAGCGGTCGCAAATGCAAC | 16 |
| ASH2L_2 | GCAGCCGCTCCTCCTGGAGA | 17 |
| ASH2L_3 | GTGGCCGTGATGGCGGCGGC | 18 |
| BRD7_1 | GTCGGACAAACACCTCTACG | 19 |
| BRD7_2 | GGGCTTCCGCTCTTTCCCAG | 20 |
| BRD7_3 | GCAGGCCCAGGCCGGCGAAG | 21 |
| BRD9_1 | GCTGGCACCCGGTCGGACCT | 22 |
| BRD9_2 | GAGTGGCGCTCGTCCTACGA | 23 |
| BRD9_3 | GCGAGCGCGGGCGGCCAGCC | 24 |
| CBX2_1 | GTACTCCAGCTTGCCCTGCG | 25 |
| CBX2_2 | GCTGAGCAGCGTGGGCGAGC | 26 |
| CBX6_1 | GTGGGTGCCGCTGAGCAAGA | 27 |
| CBX6_2 | GCTGTCTGCAGTGGGCGAGC | 28 |
| CBX6_3 | GCATCGAGTACCTGGTGAAA | 29 |
| CBX7_1 | GCTGTCAGCCATCGGCGAGC | 30 |
| CBX7_2 | GTGCGGAAGGTGAGGCTGCC | 31 |
| CBX7_3 | GCACCGCTCCCTCCACGCTG | 32 |
| CBX8_1 | GCTCCTGGAAGCGGCCAAGG | 33 |
| CBX8_2 | GGTGGGGGAGCGGGTGTTCG | 34 |
| CBX8_3 | GCACGGAGGCCCTAGGCCCG | 35 |
| CHD3_1 | GCTCCCACTCGGGCTTGGGG | 36 |
| CHD3_2 | GTCTGCCGCCTTCATCACAC | 37 |
| CHD3_3 | GAGGAAAAGAAATCCTCAGC | 38 |
| CHD3_4 | GTTTTAGGCTACTTGGGAGG | 39 |
| CHD4_1 | GCTCCGGCTCCTCCTCGCCG | 40 |
| CHD4_2 | GCGCGACCTGCGGCGGCTCC | 41 |
| CHD4_3 | GGCCGTGAGGGGCGTCTCTT | 42 |
| CNOT1_1 | GTCGAGGAGAGCCGGAGTCG | 43 |
| CNOT1_2 | GGAGCCGCCTGAGGTGAGGC | 44 |
| CNOT1_3 | GTTTCTCTACAAAATGGCGC | 45 |
| CNOT2_1 | GAGCCTAGGGGAGTGGAGTC | 46 |
| CNOT2_2 | GCCGCCTTCTCTTCTCCCCC | 47 |
| CNOT2_3 | GCAGCTCCAGATCCTAGGCC | 48 |
| CNOT3_1 | GTCAGCTTCCGCGGAGCCAT | 49 |
| CNOT3_2 | GTTGTTCTGACGACGGGGGT | 50 |
| CNOT3_3 | GCCGCTATCGCGATAGCGCC | 51 |
| CTR9_1 | GTGAGTGACGGCTCCGGCTC | 52 |
| CTR9_2 | GGAGACTACCGGCTGCGGAG | 53 |
| CTR9_3 | GATGGAGCCCCGCGACATGA | 54 |
| CXXC1_1 | GAATGAATACAACTTGATCC | 55 |
| CXXC1_2 | GAACCTCTCTGCCTGACAAA | 56 |
| CXXC1_3 | GGACGGCTGTGTGCCTTGCG | 57 |
| DMAP1_2 | GGCCGTTAGGAACATCCAAG | 58 |
| DMAP1_2 | GCGGGCCAAGAGGAGAAGGG | 59 |
| DMAP1_3 | GACCCAGGTGCGGAAGTGCG | 60 |
| DPY30_1 | GAGTGGGACAGTCCACGACT | 61 |
| DPY30_2 | GTGCTCCCGCGCCCAGGTGG | 62 |
| DPY30_3 | GATTTCAACACGAAGACTCC | 63 |
| EED_1 | GAGAAGAGGCGAAACTCAAA | 64 |
| EED_2 | GCTGAAACGTCTTTGGAAGG | 65 |
| EED_3 | GTAAGGTCCGTTGGATTAAG | 66 |
| ELP2_1 | GGACTCCCCGCACCCGGTTT | 67 |
| ELP2_2 | GTCATAGAGCACCACGGAGC | 68 |
| ELP2_3 | GGTGCCACCATGTCGCCAAC | 69 |
| ELP3_1 | GAAGCGGAAAGGTGCGAAAG | 70 |
| ELP3_2 | GCCTGGGCGTTCGCCCCTTT | 71 |
| ELP3_3 | GCAGCCACAAACTCAGACCA | 72 |
| ELP4_1 | GCCAGCGTGACCAACGACAG | 73 |
| ELP4_2 | GGTAGTGTTGCCGCGAGTAC | 74 |
| ELP4_3 | GCAACGTCACCAGTTTCCAG | 75 |
| EP400_1 | GCGTCAGGAGGGCGGGAGGA | 76 |
| EP400_2 | GGTAAGTGAGGGCGGAGGCG | 77 |
| EP400_3 | GGCTACGCGACCCCGGACCC | 78 |
| EPC1_1 | GGCACTAACACCAGCCGGGA | 79 |
| EPC1_2 | GCTGCCGGGGACTTGAGGGG | 80 |
| EPC1_3 | GTTGGCTGAAGAGCGCACAG | 81 |
| EZH1_1 | GTGAGTAAACAAGCCTGGGC | 82 |
| EZH1_2 | GGAAATTGGAAGGAATCCGA | 83 |
| EZH1_3 | GGCGCCCCTCCTCATTCCGA | 84 |
| EZH2_1 | GGATTTCGGGGTGCGTCGTG | 85 |
| EZH2_2 | GCTGCCCTCGCCGCCTGGTC | 86 |
| EZH2_3 | GGGGATGTACACAATGAAGT | 87 |
| HCFC1_1 | GAAAGGAGCAACAAGCGCCG | 88 |
| HCFC1_2 | GGGCTACGACTGAGGAAGGG | 89 |
| HDAC1_1 | GGGACGGGAGGCGAGCAAGA | 90 |
| HDAC1_2 | GGCTGAGGCTGGAGCGCCGA | 91 |
| HDAC1_3 | GCTCGGAGAGGAGGCTGCGA | 92 |
| HDAC2_1 | GGCTCGGTACCACCCGGCAG | 93 |
| HDAC2_2 | GGCGATAGTCCCGCGGGGAA | 94 |
| HDAC2_3 | GGCACCAACTCGCGAGGAGG | 95 |
| IKBKAP_1 | GTTTGGGCAGATGGGCAAGA | 96 |
| IKBKAP_2 | GCCTGGCACCGTAGAGGTAG | 97 |
| IKBKAP_3 | GGCGAGGCCGGGCCCGCTTC | 98 |
| ING3_1 | GAGGGAACAAGGGGGTCCAG | 99 |
| ING3_2 | GGAAAGTGAGTGCGCGGCGC | 100 |
| ING3_3 | GAGTTTTGTCCCCTCCAATA | 101 |
| INO80_1 | GGGGTCCCAGGAGCCGCGGA | 102 |
| INO80_2 | GGTTCGCTCTCTGAGGCCGT | 103 |
| INO80B_1 | GAAAGGGGACTAGAAATGGT | 104 |
| INO80B_2 | GCGGCGTGGGAGCACCTCTG | 105 |
| INO80B_3 | GCGAATAGATCAAGCAATTT | 106 |
| INO80C_1 | GAAGACTCGGAGTGCGATGG | 107 |
| INO80C_2 | GTTCCGGACTATTCCGGGAG | 108 |
| INO80C_3 | GGAAGTTCCAAGGCCCGCGC | 109 |
| INO80D_1 | GGCTGACAGATCAGAGTGAG | 110 |
| INO80D_2 | GGAGCCCGGGGATGTGGGCC | 111 |
| INO80E_1 | GGTAGCGGGAGGGCAGACTC | 112 |
| INO80E_2 | GTCATGAACGGGCCGGCGGA | 113 |
| INO80E_3 | GTGCTGCCGCGGGAAGGCTG | 114 |
| JARID2_1 | GACTCGGCGAGCCCTCGCTG | 115 |
| JARID2_2 | GTTACATCTTGGAAAAGAAA | 116 |
| JARID2_3 | GGGGGGGGAGTGAAGGGCGT | 117 |
| KAT5_1 | GCAAGACTGCCCCTGTGACT | 118 |
| KAT5_2 | GCCTCACGAAGCCCCTGTAG | 119 |
| KAT5_3 | GCCACTGGCTGTGCACGTTA | 120 |
| KDM1A_1 | GACAGAGCGAGCGGCCCCTA | 121 |
| KDM1A_2 | GGCGGCCCGAGATGTTATCT | 122 |
| KDM1A_3 | GCGTGAAGCGAGGCGAGGCA | 123 |
| KDM2B_1 | GCTCGGCTTCCATACCTATA | 124 |
| KDM2B_2 | GCGGACCCGCCATGTGGAGG | 125 |
| KDM2B_3 | GTCGGCCACACAGGTAATGT | 126 |
| KDM6A_1 | GCAGCCACAGGCGGGGACGG | 127 |
| KDM6A_2 | GAAAGCCGCCGCTGCCGACC | 128 |
| KDM6A_3 | GGAGCACTGAGGGGATTCGT | 129 |
| KMT2A_1 | GAGGCGGCGGCCGCTCCCCC | 130 |
| KMT2A_2 | GGCCGGCCCTGAAGAGGCTG | 131 |
| KMT2A_3 | GGCGCTTCCCCGCCCGACCC | 132 |
| KMT2D_1 | GATAAAGATTCAGAACCGGC | 133 |
| KMT2D_2 | GTGCCAGGACCAGAAATGTA | 134 |
| KMT2D_3 | GAGATTATCCAAAACCTGAG | 135 |
| LEO1_1 | GTGAGCGATAATGGCGGATA | 136 |
| LEO1_2 | GCGAAGCTGAGCGTAAAGGT | 137 |
| LEO1_3 | GCGTGGCAGGCCTTCCGCTG | 138 |
| MBD2_1 | GGATTCCAAGGGCTCGGTTA | 139 |
| MBD2_2 | GGGCTGGATGCGCGCGCACC | 140 |
| MBD2_3 | GGACCTAAGAGGCGGTGGCC | 141 |
| MBD3_1 | GGAAGAAGTGCCCAGAAGGT | 142 |
| MBD3_2 | GAGCCCGTTGAGGCCCTGCG | 143 |
| MBD3_3 | GCGCAATGGAGCGGAAGAGG | 144 |
| MED1_1 | GATCAATCTGAAGTCCCCGG | 145 |
| MED1_2 | GGCTCGGGATCCCGGGACGC | 146 |
| MED1_3 | GAAGCTAGATCCGCCACAAA | 147 |
| MED10_1 | GGAGAAGTTTGACCACCTAG | 148 |
| MED10_2 | GTTGAGCCCGGCCTGGCTGC | 149 |
| MED10_3 | GGTCTCCCCAGGGCCTGGCC | 150 |
| MED12_1 | GCGGCCGAGAGACAACAAGG | 151 |
| MED12_2 | GAGGGAGCCGAAAAGGGGGG | 152 |
| MED12_3 | GTAGCGCCGGAGGCACCAGC | 153 |
| MED13_1 | GCCGGCGGCGGCTGCTGTGA | 154 |
| MED13_2 | GGTTACAGTGACAATCTTCC | 155 |
| MED13_3 | GGTGCGCCCTTGGGCCGTGG | 156 |
| MED14_1 | GACTCTGCCCGCTCCCGTTT | 157 |
| MED14_2 | GTGTGCCGTTGCGCCAAGCC | 158 |
| MED14_3 | GTGGTTCTCCAGCTGCACTG | 159 |
| MED15_1 | GATACGGGCGGCGGGAGCTG | 160 |
| MED15_2 | GGTCAGTCAAATGTGAGTAG | 161 |
| MED15_3 | GCCGCCTCAGTCACAGAGCC | 162 |
| MED17_1 | GGGAGCTTGCGGTGCGTTCT | 163 |
| MED17_2 | GCGTTGCGTTCGGTTTCCCG | 164 |
| MED17_3 | GAGGCTTCCCTGCGGAGAGC | 165 |
| MED23_1 | GGAATATAGGGGCAGAGGGG | 166 |
| MED23_2 | GGCGGGGGTGATAGTACAGA | 167 |
| MED26_1 | GGCGGCTCCTCCTCCTCCTT | 168 |
| MED26_2 | GTCACTCACTCGCCGGCCTC | 169 |
| MED26_3 | GGCGTCTCCGCAGCAGATCA | 170 |
| MED4_1 | GCGGCTGCTGTCTGCGCTTG | 171 |
| MED4_2 | GGCGAGCCTGAGAGCCGGGC | 172 |
| MED4_3 | GGAGCGGCTGGGAGGCGGTT | 173 |
| MED6_1 | GTTTCGCTAGATCACAGCCT | 174 |
| MED6_2 | GATTGTCTGTGGACCAGTTT | 175 |
| MED6_3 | GCGTTTACAGGTTCTCTTTC | 176 |
| MED7_1 | GAAAGACGAAAGACCGCCTT | 177 |
| MED7_2 | GTGCGGTCTCTCCGAGAGCG | 178 |
| MED7_3 | GGCTCTAAGCGTGGCAGTCT | 179 |
| MED8_1 | GACCGAGAGTGGGCTGGCTA | 180 |
| MED8_2 | GGCAGAACCCACGGCTGATA | 181 |
| MED8_3 | GCGTTGGGCGTACTAGCGGC | 182 |
| MEN1_1 | GTGGGATGTAAGCGCGGAGG | 183 |
| MEN1_2 | GACAGACTTTACAGCCCCGG | 184 |
| MEN1_3 | GGACTCTCCTTGGGGTTTGG | 185 |
| MRGBP_1 | GCTCGGCCGGGCCGCGGCCA | 186 |
| MRGBP_2 | GCCGCAGGCGACAAGGGCCC | 187 |
| MRGBP_3 | GACAGTGGTGTGGAGCCCCG | 188 |
| MTA1_1 | GCCGCCAACATGTACAGGGT | 189 |
| MTA2_1 | GTTGGGCTCTGCCGGCCGCA | 190 |
| MTA2_2 | GAACGAGCTCGGCTCCTGCC | 191 |
| MTA2_3 | GCCTCAGCGTCCCGGAGTG | 192 |
| MTA3_1 | GCCCCAGAACGTGGGGGCCG | 193 |
| MTA3_2 | GTCCAGGCGCGCTACACGTT | 194 |
| MTA3_3 | GGGGAGGAACGCCTTGTCAC | 195 |
| NCOA6_1 | GTCGGGCTGGCTTCGCGGGG | 196 |
| NCOA6_2 | GACCGTGCCACTCGGTCGCC | 197 |
| NCOA6_3 | GACGGCGGCGCGGGCCCGTA | 198 |
| OGT_1 | GCTCTGGAGGGCTTGAGCGG | 199 |
| OGT_2 | GCTCCAGATGGCGTCTTCCG | 200 |
| OGT_3 | GATGGTCAATTAGAGTTCCC | 201 |
| PAF1_1 | GTGAACGCGCAGGCAGCACC | 202 |
| PAF1_2 | GCGGAAAGTGGGTTGAGATG | 203 |
| PAF1_3 | GCGGCCTGAGGAGACCCGTT | 204 |
| PBRM1_1 | GGGTAAGGCCGGGCCCAGGG | 205 |
| PBRM1_2 | GGCCCGGCAGCTGACCAAGG | 206 |
| PBRM1_3 | GCAGGTGCGACAAGGCTACT | 207 |
| PCGF1_1 | GCCTCATCGCGATCGCAATC | 208 |
| PCGF1_2 | GATGGACCCGCTACGGAACG | 209 |
| PCGF1_3 | GTCGGCCAGCGGTGCGAATT | 210 |
| PCGF2_1 | GCTTACCTGGGTTCGGGGTC | 211 |
| PCGF2_2 | GCCTGTAACCCTCTGGGGAT | 212 |
| PCGF2_3 | GGGGGGTGCGAAGGCAGGAT | 213 |
| PCGF6_1 | GTAGGCGCTGCCAAAACCGA | 214 |
| PCGF6_2 | GGCGCCTCTGTCTGAGACGG | 215 |
| PCGF6_3 | GGTGTCTCTCCCGACCATGG | 216 |
| PHC1_1 | GAAGGTAACCGGGCGACCGA | 217 |
| PHC1_2 | GGGCGTTACACAGATGGAGG | 218 |
| PHC2_3 | GCTCAGCGCCGGAGGTAGGC | 219 |
| PHC2_1 | GACTGGCAGCTCATTCTCCA | 220 |
| PHC2_2 | GTACACAGAAATCTGGGGCC | 221 |
| PHC2_3 | GGTAAGAGTCTAATTGATCT | 222 |
| PHC3_1 | GTGACTGATGTCGTAACTAG | 223 |
| PHF10_1 | GGGCCCACGCCCCGGCACCC | 224 |
| PHF10_2 | GTCGCTGTCGCACGGCCGCG | 225 |
| RBBP4_1 | GGCACCCTCACCTTCCTTGT | 226 |
| RBBP4_2 | GCTGAGCCGCGGCCTCGACA | 227 |
| RBBP4_3 | GGGGGCGCAGGAAACAATAG | 228 |
| RBBP5_1 | GTTGTTGCCGGAGCTGAGAC | 229 |
| RBBP5_2 | GCTGCGTTTTAGAGAAGCGT | 230 |
| RBBP5_3 | GGTGGACGCCGCGAAGAGAC | 231 |
| RBBP7_1 | GGAGCGCAGCCGCTGGAGGA | 232 |
| RBBP7_2 | GCGCGCGCGTTGACCGCCTC | 233 |
| RBBP7_3 | GCCCTTGTCCGGGGGTTGCT | 234 |
| RTF1_1 | GGCGGGCAAGAGGGGAGTCC | 235 |
| RTF1_2 | GGACCACCATGGTAAAGAAG | 236 |
| RTF1_3 | GCGCGGGCCGGCGGAGCCAG | 237 |
| RUVBL1_1 | GGGCGCACTGTCCTAGCTGC | 238 |
| RUVBL1_2 | GCCTCCCACAGCCACGTGAA | 239 |
| RUVBL1_3 | GCAGGCGGCCTCAGGGCTTG | 240 |
| SAP18_1 | GGTCAGGGCGAGCGTCTCGC | 241 |
| SAP18_2 | GGAGTCGCGCGTTACCCAGG | 242 |
| SAP18_3 | GATCGACCGCGAGAAGGTGA | 243 |
| SAP30_1 | GTGAGCGGGGTCCCCGCTCC | 244 |
| SAP30_2 | GGCCCGGGACAGTTGGTGTT | 245 |
| SAP30_3 | GCAGAGTGAATTGCCGCTGC | 246 |
| SETD1A_1 | GAATAGCCCGCTTCTGTCCC | 247 |
| SETD1A_2 | GCCAGCAGGGATTGGCTAAC | 248 |
| SETD1A_3 | GACTCCACCAAGGCGGATGA | 249 |
| SETD1B_1 | GGTTCCTCCTCTCGCCCGAA | 250 |
| SETD1B_2 | GATTGACCCGGCTCTGAAAA | 251 |
| SETD1B_3 | GCACGGCTGGGGGGGCGCGC | 252 |
| SIN3A_1 | GGGCTAGTCCGCCGGCCGCT | 253 |
| SIN3A_2 | GCTCGGTCCCAGGGCCCGCA | 254 |
| SIN3A_3 | GGCCTGTCCCTCGCCTACCT | 255 |
| SIN3A_4 | GCGGCCGCTTCTCTGTTACC | 256 |
| SIN3A_5 | GCCTGTGACCGCTTCGTTAG | 257 |
| SIN3B_1 | GGGACGCCACTCACGTGCAC | 258 |
| SIN3B_2 | GAGGGCCGAGGTGAGAGGTG | 259 |
| SMARCA4_1 | GGGCGGTTTGAATGGAGCCG | 260 |
| SMARCA4_2 | GGCGCGCCCTGTGCGGGGCC | 261 |
| SMARCA4_3 | GGGAAGGCCACAGTGTCGCG | 262 |
| SMARCB1_1 | GGCCTGGTCGTCGTCTGCGG | 263 |
| SMARCB1_2 | GGGCCGAGGGAAACCGAAGC | 264 |
| SMARCB1_3 | GCGAGGGATCAGGAGGGCTG | 265 |
| SMARCC1_1 | GCTGTTTATCGACGGAAGGA | 266 |
| SMARCC1_2 | GACGGTGTCCCAGCTGGATT | 267 |
| SMARCC1_3 | GGTGGGTTCGCGCGCCCGTG | 268 |
| SMARCC2_1 | GACAACGTGCGGCTGTGGCT | 269 |
| SMARCC2_2 | GACCGCGGCCCTGCAGCCCC | 270 |
| SMARCC2_3 | GCCTCGTAGTACTTCACGTT | 271 |
| SMARCD1_1 | GTGGCTCCAAGCGGCGGCGC | 272 |
| SMARCD1_2 | GCCGCACAAAGAACCGGAAC | 273 |
| SMARCD2_1 | GACTCGGGCGGCCAAACCTC | 274 |
| SMARCD2_2 | GCCCGGGAGATTCCGGATCC | 275 |
| SMARCD2_3 | GGAACTCGCGAACTTGGATT | 276 |
| SMARCD3_1 | GAATGGGAGTCTGCCAGTCA | 277 |
| SMARCD3_2 | GCCAGGCAGCGATGGGGAGG | 278 |
| SMARCD3_3 | GAAAGTGCTCGGCAGGGGGG | 279 |
| SMARCE1_1 | GCGGGTGAGTGTTTCCAAGT | 280 |
| SMARCE1_2 | GAACTCGGGGTCTAGCCAAG | 281 |
| SMARCE1_3 | GGCCTCAAGGAGGCCTCAAC | 282 |
| SRCAP_1 | GTCAGTCCGTCGGGAGGGCT | 283 |
| SRCAP_2 | GCTCGGGTCTTGGGAACGTG | 284 |
| SRCAP_3 | GTGTGAACCCGCAGGAGGCC | 285 |
| SUZ12_1 | GGGCGAGCGGTTGGTATTGC | 286 |
| SUZ12_2 | GGCGGGTAGCTGGCGGGGGG | 287 |
| SUZ12_3 | GCCTCAGAAGCACGGCGGTG | 288 |
| THAP1_1 | GTGATGGTGGCCTCCCTCGG | 289 |
| THAP1_2 | GTTCTCAGTGTCGCTGCGCT | 290 |
| THAP1_3 | GCTAATGCAAACAACAAAAC | 291 |
| THAP3_1 | GCTGCCCCCAACAAAGATGG | 292 |
| THAP3_2 | GGGTCCCCGCCTCTTACCGG | 293 |
| THAP3_3 | GGGCCCGCGGACCGACTCCG | 294 |
| THOC1_1 | GCTTCGGGCAAACTGAAGAG | 295 |
| THOC1_2 | GGCAAAATTCGAGTAATTTC | 296 |
| THOC1_3 | GTCCGCCTCAGCGTCCGCTC | 297 |
| THOC2_1 | GAGGCGAATTGTGAGTGTTC | 298 |
| THOC2_2 | GCTGCACTCTCACCTGTAGT | 299 |
| THOC2_3 | GACCATCCACGCCCGCCGCC | 300 |
| THOC3_1 | GCTGCTGCAGTGTTGTGAGT | 301 |
| THOC3_2 | GGCGGTCCCCGCTGCAGCCA | 302 |
| THOC3_3 | GCCCCGGCTCGATGGCCCCG | 303 |
| TRRAP_1 | GGGTCGCGGGCCGGGCCTGC | 304 |
| TRRAP_2 | GGCGGGCGTCCGAACGGCCC | 305 |
| TRRAP_3 | GCGGCCGAGCGGTTGCGACG | 306 |
| WDR5_1 | GGCCGCACAGGAGACAAGGG | 307 |
| WDR5_2 | GCTCTGGCGGCCTCGGTCTC | 308 |
| WDR5_3 | GGCACGCACCTTGCTCTGAG | 309 |
| WDR82_1 | GAGGTGGCTGTGAGGACGAA | 310 |
| WDR82_2 | GGAGGAGGCGGCCCAACTGT | 311 |
| WDR82_3 | GCGGAGCTTCCGCGTCGCTA | 312 |
| DC13_1 | GGGCTGAACGCGTATTCGCG | 313 |
| DC14_1 | GGCGATTCGGCGACCTTAGT | 314 |
| DC14_2 | GGCGCGAGTACGAAATTAAT | 315 |
| DC14_3 | GATTATCAGACGCGCTGCGT | 316 |
| DC15_1 | GCGCGGCTAGAATAGACTTG | 317 |
| DC15_2 | GGTTCGTGCGGTAGTGTGCG | 318 |
| DC15_3 | GTATCGTCTTCCGTCCTCGT | 319 |
| DC15_4 | GGCTACTCTATGCGTCGATT | 320 |
| DC15_5 | GCTTAACAAAGCGAGCGACC | 321 |
| DC15_6 | GGCACTGGACGATATCCGAC | 322 |
| DC15_7 | GTTCATCTCAACGGTAATCG | 323 |
| DC1617_1 | GGTTATATTGACGTCCTGCC | 324 |
| LC_1 | GCTAGTCTGCGTGACGCGTCT | 325 |
| LC_2 | GAAGTAACTGAAGGATCAATAT | 326 |
| LC_3 | GCGGGAAAACCGCGCCCCGGA | 327 |
| LC_4 | GCTCAGGGCCGTGACGCGTGGG | 328 |
| LC_5 | GTAGGAGCGCGTGCTGATTGT | 329 |
| LC_6 | GGACGAACTAATGTATTGTGGC | 330 |
| LC_7 | GGTTTATGGACCTTCAGGGAG | 331 |
| LC_8 | GGCGTACCCGTGGTTTCACCGT | 332 |
| LC_9 | GCTTGGGAGCAAGCCGGCGGTA | 333 |
| LC_10 | GTGTGGCGACCCTGGTCTCAT | 334 |
| LC_11 | GGGCCTCTGTGAGGTCGTGGT | 335 |
| LC_12 | GTATGATACTCGTGCTTAGT | 336 |
| LC_13 | GCGAAGTCGAATGTTGGTCG | 337 |
| LC_14 | GGCCCAACATCCTCGTGTCCA | 338 |
| LC_15 | GTGGCGGAGCCTAGCCGAGAGT | 339 |
| LC_16 | GGCGCGAACTTTAAGGTGGAC | 340 |
| LC_17 | GATTAGTTCGCGTATGGCAGCA | 341 |
| LC_18 | GCCGTAAGGACGGGTAGAGGT | 342 |
| LC_19 | GGGGGCGGAAATCGAGCCCT | 343 |
| LC_20 | GAAGTGAGAGGAGGGAGCAGCC | 344 |
| LC_21 | GTAAATCCCGGAGTCAGA | 345 |
| LC_22 | GTGAGCGGCGACCCCCCCTG | 346 |
| LC_23 | GGTGCGGACCCCCGCCGGGGG | 347 |
| LC_24 | GGTGAGCCGGTTTGTGAGAAG | 348 |
| LC_25 | GAGAGTGCGCTGCAATGGATAT | 349 |
| LC_26 | GGATGTGCCATGGTGAGGGCTG | 350 |
| LC_27 | GGATGCGCCTAGGCGAAAGAAA | 351 |
| LC_28 | GAGCCGATGCAGGGCGTAGGG | 352 |
| LC_29 | GCCATTCTCTATGTTCGATAAG | 353 |
| MCM2_PC | GGATCGTGGTACTGCTATGG | 354 |
| INTS9_PC | GGCAGGTGGCGGAGATTGCAC | 355 |
| GEMIN5_PC | GGCGTGAGGCTACGAGCGGT | 356 |
| CENPA_PC | GCCAAGCACCGGCTCATGTG | 357 |
| POLR1D_PC | GGAAGCAAGGACCGACCGA | 358 |

TABLE 6
Regular primers for cloning and sequencing
primers to clone single sgRNA:

| | |
|---|---|
| Forward | GGAGAACCACCTTGTTGGN19GTTTAAGAGCTATG |
| primer | CTGGAAACAGCA (SEQ ID NO: 359) |
| (N19 is the | |
| targeting | |
| sequence): | |
| Reverse | CTAGTACTCGAGNNNNNNNNNNGCGTCGACCCTAG |
| primer | GGCTAGCACTAGTAAAAAAAGCACCGACTCGGTGC |
| (N10 is the | CAC (SEQ IDNO: 360) |
| barcode | |
| sequence): | |

PRIMERS TO AMPLIFY GENOMIC DNA FOR SINGLE
SCREENS:

| | |
|---|---|
| Forward | AATGATACGGCGACCACCGAGATCTACACGGTAAT |
| primer: | ACGGTTATCCACGCGG (SEQ ID NO: 361) |
| Reverse | CAAGCAGAAGACGGCATACGAGATNNNNNNNNGCA |
| primer | CAAAAGGAAACTCACCCT (SEQ ID NO: 362) |
| (NNNNNNNN | |
| is the | |
| index): | |

CUSTOM PRIMERS FOR M1SEQ:

| | |
|---|---|
| Read2 | GTGTGTTTTGAGACTATAAGTATCCCTTGGAGAAC |
| primer: | CACCTTGTTGG (SEQ ID NO: 363) |
| Index read | GTCTCAAAACACACAATTACTTTACAGTTAGGGTG |
| primer: | AGTTTCCTTTTGTGC (SEQ ID NO: 364) |

PRIMERS TO AMPLIFY GENOMIC DNA FOR DOUBLE
SCREENS:

| | |
|---|---|
| Forward | AATGATACGGCGACCACCGAGATCTACACTGAGAC |
| primer: | TATAAGTATCCCTTGGAGA |
| | (SEQ ID NO: 365) |
| Reverse | CAAGCAGAAGACGGCATACGAGATNNNNNNCTGGC |
| primer | GAACTACTTACTCTAGCTTCCCGGCAACGCCTTAT |
| (NNNNNN | TTAAACTTGCTATGCTGT |
| is the | (SEQ ID NO: 366) |
| index): | |

CUSTOM PRIMERS FOR H1SEQ-2500:

| | |
|---|---|
| Read1 | CGAAGTTATAAACAGCACAAAAGGAAACTCACCCT |
| primer: | AACTGTAAAGTAATTGTGTG |
| | (SEQ ID NO: 367) |
| Index read | GTTTAAATAAGGCGTTGCCGGGAAGCTAGAGTAAG |
| primer: | TAGTTCGCCAG (SEQ ID NO: 368) |
| Read2 | GCACCGACTCGGTGCCACTTTTTCAAGTTGATAAC |
| primer: | GGAC (SEQ ID NO: 369) |

TABLE 7
qPCR primer sequences

| Gene | | |
|---|---|---|
| name | Forward primer | Reverse primer |
| SIN3B | TTACTGCATGTCCAAGTTCAAGA | CCAGGTGTCGTTCAGTA |
| | (SEQ ID NO: 370) | CCC |
| | | (SEQ ID NO: 371) |
| MED4 | GGTGGTAACAGCACACGAGA | TTGCCAGCATTTCTATA |
| | (SEQ ID NO: 372) | AGTTCC |
| | | (SEQ ID NO: 373) |
| MED6 | TGCAGAGGCTAACATTAGAACAC | GCTGTTGCTTCCGAATG |
| | (SEQ ID NO: 374) | ATGA |
| | | (SEQ ID NO: 375) |
| MRGBP | TGAACCGACACTTCCACATGA | TGGTCCCAGATGACCTT |
| | (SEQ ID NO: 376) | GGAT |
| | | (SEQ ID NO: 377) |

### Example 3

### Repression Screening Platform

Besides gene activation, gene repression also can facilitate cell fate conversion. For example, knockdown of many epigenetic modulators increases the efficiency of reprogramming or transdifferentiation processes. This example describes, a repression screen platform to identify cell fate conversion barriers genes.

To perform gene repression screens, a clonal mouse ES cell line carrying Staphylococcus aureus (SaCas91-KRAB is co-transfected with Cas9, sgRNA targeting mouse Rosa 26 loci, and a vector containing dCas9-KRAB with a Zeocin-resistance gene. Zeocin-resistant cells are sorted into a 96-well plate. After a week of culture, the genome is purified and the correct integration of SadCas9-KRAB into Rosa 26 loci is confirmed. This clonal cell is used as a platform to identify gene barriers of differentiation processes.

To perform single gene repression screens, a genome-wide gene repression SadCas9 sgRNA library is generated. The library includes sgRNAs targeting −50 bp to +300 bp region relative to all putative genes in the mouse genome. All the available sgRNAs are blasted through mouse genome and excluded if there is predicted off-target binding. Other design criteria and construction method are similar to the design of activation sgRNA library described in Example 1. This repression library is transduced into the SadCas9 repression mouse ES cells, and neural differentiation is performed as in the single screen. On day 12, cells are harvested and sorted for hCD8+ and hCD8−. The sgRNAs are sequenced, paired-analyzed for enriched genes in hCD8+ and hCD8− populations, and a list of top hits for neural differentiation barrier genes is identified.

Over the past years, the literature has shown that the activation of combinatorial transcription factors can control a cell fate. For example, the transcription factors Oct4, Klf4, Sox2, and c-Myc are used to reprogram somatic cells to induced pluripotent stem (iPS) cells. Moreover, activation of combinatorial transcription factors also induces the generation of many cell types, such as cardiomyocytes, neurons, and hepatocytes, directly from somatic cells. These works indicate that single TFs are not sufficient to achieve a cell fate conversion process in most cases. Thus, a platform that allows combinatorial screen is in urgent need to facilitate cell fate determination studies.

To perform a second-round combinatorial activation screen, an sgRNA library that achieves double gene activation is generated. In this library, two different sgRNA cassettes are constructed into one vector. The first cassette contains sgRNAs targeting top hit genes from the single activation screen, which are driven by a human U6 promoter. Meanwhile, each vector contains the second cassette, which is a sgRNA with a different stemloop sequence driven by a mouse U6 promoter. The sgRNAs of the second cassettes also target top hit genes from the first round activation single screen. This construct expresses sgRNAs targeting two different genes, as well as avoids recombination of repeated sgRNA sequences. Two different sgRNAs bind to dCas9 and achieve the activation of two different top hit genes simultaneously in the dCas9-activation system. This allows the combinatorial double activation screen.

In some embodiments, this double activation library is transduced into CamES cells, and neural differentiation is performed as in the single screen. On day 12, cells are harvested and sorted for hCD8+ and hCD8−. The sgRNAs are sequenced and paired-analyze enriched genes in hCD8+ and hCD8− populations are identified. The screen identifies optimal TF combinations that drive neural differentiation of mouse ES cells.

Additionally, the combination of gain-of-function and loss-of-function techniques accelerates cell fate conversion, and sheds light on the fully revelation of cellular reprogramming mechanisms. However, a platform to perform gain-of-function and loss-of-function screen simultaneously is not available at present.

To perform a simultaneous activation/repression screen, a clonal ES cell line carrying gene activation/repression cassettes is generated. Vectors containing two cassettes separately are constructed. One vector contains the activation cassette, which is a dead Streptococcus pyogenes Cas9 (SpCas9)-activation system, with a eGFP gene cassette. The other vector comprises SadCas9-KRAB, with a zeocin-resistance gene cassette following. The two vectors, together with Cas9 and sgRNA targeting mouse Rosa26 loci are co-transfected into mouse ES cells. To select mouse ES cells carrying these two system, transfected ES cells are selected with zeocin. After seven days, remaining zeocin-resistant cells are analyzed with flow cytometry and single GFP+ cells are sorted into 96-well plates. One week later, the genome of clonal cells is analyzed to confirm the correct integration of both activation and repression cassettes. This clonal cell line allows the activation and repression of different genes simultaneously.

An sgRNA library that achieves gene turning-on and -off simultaneously is constructed. In this library, two different sgRNA cassettes are constructed into one vector. The first cassette contains sgRNAs of SpCas9 targeting top hit genes from the single activation screen, which are driven by a human U6 promoter. Meanwhile, each vector contains the second cassette, which is a sgRNA of SaCas9 driven by a mouse U6 promoter. The sgRNAs of SaCas9 in the second cassettes target top hit genes from the first round repression screen. This construct expresses sgRNAs of SpCas9 and SaCa9, and thus allows simultaneous gene activation and repression.

This activation/repression library is applied to clonal turning-on/off mouse ES cells, and neural differentiation is performed as in the single screen. On day 12, cells are harvested and sorted for hCD8+ and hCD8−. The sgRNAs and paired-analyze enriched genes in hCD8+ and hCD8− populations are sequenced. A series of gene combinations having both TF determinants and neural differentiation barriers is identified. The simultaneous turning-on of IT determinants and turning-off of neural differentiation barriers generates very high efficiency of neural cells of mouse ES cells.

### Example 4

### Experimental Procedures

### Plasmid Design and Construction

To clone sgRNA vectors, the optimized sgRNA expression vector (pSLQ1373) was linearized and gel purified (Chen et al., 2013). New sgRNA sequences were PCR amplified from pSLQ1373 using different forward primers and a common reverse primer, gel purified and ligated to the linearized pSLQ1373 vector using In-Fusion cloning (Clontech). Primers used to construct individual sgRNAs are shown in Table 8. To change the promoter of scFv-sfGFP-VP64, the EF1α and PGK promoters were PCR amplified, gel purified, and ligated to linearized pSLQ504 using In-Fusion cloning (Clontech).

Two-guide expression vectors were assembled by a two-step cloning procedure. First, new sgRNA sequence (integrated DNA Technologieds) were PCR amplified from pSLQ5004 and ligated into BstXI and XhoI-digested pSLQ5004 parental vector, which contained a modified human 136 promoter (hU6). The same single sgRNA expression constructs were cloned into pSLQ1373 as previously described, which contained a modified mouse U6 promoter (mU6) and an optimized stem loop sequence of sgRNA. Second, the two-guide expression cassettes were then assembled from PCR amplified single cassettes using two sgRNA forward and reverse primers from pSLQ5004-based single sgRNA constructs and inserted into NsiI-digested pSLQ1373 single sgRNA constructs. Primers used to construct individual sgRNAs are shown in Table 11.

sgRNA Library Design

Putative transcription factor (TF) genes were selected according to the TRANSFAC database, and TSS (transcription start site) for each gene was determined using the Gencode and refFlat databases. All possible transcripts were selected if multiple TSSs exist for a gene. All sgRNAs targeting −3 kb to 0 relative to TSS were kept. Using the CRISPR-era algorithm (Liu et al., 2015), the targeting sequences of sgRNAs adjacent to an NGG PAM (protospacer adjacent motif) were computed, starting with a G (for more efficient U6 promoter activity) with a length of 20 bp. The sgRNAs containing homopolymers spanning greater than 3 nucleotides (nt) were discarded. To avoid off-target effects, sgRNA sequences alignment to the mouse genome was computed using the short read aligner Bowtie, and those with less than 2 mismatches with another genomic region were excluded. Furthermore, sgRNA sequences that contained certain restriction sites (BstXI and BlpI) were also removed. sgRNAs with a GC content between 30% and 70% were kept. An average of about 60 sgRNAs were selected for each target gene. Sequences for non-targeting negative control sgRNAs were generated using a randomized mouse gene TSS region and selected using the same rules as described above.

sgRNA Library Construction

The oligonucleotide pool was synthesized by Custom Array. The oligo library was PCR amplified, gel purified and ligated to the linearized backbone vector (pSLQ1373) digested with BstXI and BlpI using In-Fusion cloning. Libraries and parental vector will be made available on addgene.org.

### Cell Culture

E14 mouse ES cells and CamES cells were maintained on gelatin coated tissue culture plates with basal medium (50% Neurobasal, 50% Dulbecco modified Eagle medium (DMEM)/Ham's nutrient mixture F12, 0.5% NEAA, 0.5% Sodium Pyruvate, 0.5% GlutaMax, 0.5% N2, 1% B27, 0.1 mM β-mercaptoethanol and 0.05 g/L bovine albumin fraction V; all from Thermo Fisher Scientific) supplemented with LIF (Millipore) and 2i (Stemgent). Human embryonic kidney (HEK293T) cells (ATCC) were cultured in 10% fetal bovine serum (Thermo Fisher Scientific) in DMEM (Thermo Fisher Scientific).

### Construction of the CamES Cell Line

Mouse ES cells were co-transduced with multiple lentiviral constructs that expressed dCas9-SunTag from a TRE3G promoter, scFV-sfGFP-VP64 from the EF1a or PGK promoter, and reverse tetracycline-controlled transactivator (rtTA) from the EF1a promoter. After adding Doxycycline, polyclonal cells were sorted by flow cytometry using a BD FACS Aria2 for GFP+ and mCherry+ cells. After verification of gene activation using a sgBrn2, monoclonal cells were further sorted, and one efficient cell line was chosen as CamES cells.

### Construction of the Tuj-1-hCD8 CamES Cell Line

Construction of CRISPR/Cas9 vector for Tuj1 knockin. The pX330-derived pSLQ1654 encoding the nuclease Cas9 and an optimized sgRNA sequence was first linearized by a BbsI digest and gel purified. Two primers sgTuj-1 F and sgTuj-1 R were phosphorylated, annealed, and ligated to the linearized vector pSLQ1654 to generate pSLQ1654-sgTuj1. sgTuj-1 F: caccgcccaagtgaagttgctcgcagc. sgTuj-1 R: aaacgctgegagcaacttcacttgggc.

Construction of DNA template. The Tuj1-IRES-hCD8 vector (pSLQ1760) was assembled with three fragments (5′ homologous arm of Tuj1, IRES-hCD8 and 3′ homologous arm of Tuj1) and a modified pUC19 backbone vector by using Gibson Assembly Master Mix (New England Biolabs). Both 5′ and 3′ homology arms were PCR amplified from the genomic DNA extracted from mouse ES cells with Herculase 11 Fusion DNA polymerase (Agilent). The IRES-hCD8 was PCR amplified from pSLQ1729. The backbone vector was linearized by digestion with PmeI and ZraI. All DNA fragments and the backbone vector were gel purified followed by a Gibson assembly reaction. Primers: 5′ homologous arm F: aaagtgccacctgacactcagtcctagatgtcgtgegg (SEQ ID NO:380). 5′ homologous arm R: tcacttgggcccctgggct (SEQ ID NO:381). IRES-human CD8 F: caggggcccaagtgaactagtaaaattcgcccctctccctc (SEQ ID NO:382). IRES-human CD8 R: cagctgcgagcaactttaacctgcaaaaagggagcagtaaagg (SEQ ID NO:383). 3′ homologous arm F: agttgctcgcagctggggt (SEQ ID NO:384). 3′ homologous arm R: agctggagaccgttttttctgactgactggalacagggcat (SEQ ID NO:385).

Electroporation and clonal Tuj1-hCD8 CamES cells: 2.5 μg pSLQ1654-sgTuj1, 12.5 μg Tuj1-IRES-hCD8 template DNA in 100 μL Nucleofector solution (Amaxa) were electroporated into 1×10⁶ CamES cells using program A-030. Both plasmids were maxiprepped using the Endofree Maxiprep Kit (Qiagen). After 3 days of culture, sorted single cells were seeded in a 96-well plate with one cell per well. All clonal cell lines were analyzed using PCR and sequencing (Yu et al., 2015).

### Lentiviral Production

HEK293T cells were seeded at ˜30% confluence one day before transfection. Lentivirus were produced by cotransfecting with pHR plasmids and encoding packaging protein vectors (pMD2.G and pCMV-dR8.91) using TransIT-LT1 transfection reagents (Mirus). Viral supernatants were collected 3 days after transfection and filtered through 0.45 μm strainer. Supernatant was used for transduction immediately or kept at −80° C. for long-term storage.

### Quantitative RT-PCR

Cells were harvested using Accutase (STEMCELL), and total RNA was isolated using the RNeasy Plus Mini Kit (QIAGEN), according to manufacturer's instructions. Reverse transcription was performed using iScript cDNA Synthesis kit (Bio-Rad). Quantitative PCR reactions were prepared with iTaq Universal SYBR Green Supermix (Bio-Rad). Reactions were run on a LightCycler thermal cycler (Bio-Rad). Primers used are summarized in Table 9.

### High-Throughput Pooled Neural Differentiation Screens

The neural differentiation screens were performed as two independent replicates. For both screens, 10⁸ CamES cells were seeded at 40,000 cells/cm² density at day −2. Cells were transduced with pooled lentiviral sgRNA library with an MOI of 0.3 at day −1 in basal medium supplemented with LIF and 2i. At day 0, puromycin was added at 1 μg/mL in ES2N medium (Millipore) with Doxycycline for another 24 hours. Fresh ES2N medium was changed with Doxycycline every day starting day 2. On day 12, cells were harvested and sorted for hCD8+ and hCD8− cells using EasySep human CD8 isolation kit (STEMCELL Technologies). Populations of cells expressing this library of sgRNAs were either harvested at the outset of the experiment (the day 0 time point: after 24 hours puromycin selection), hCD8+, or hCD8− cells. Genomic DNA was harvested from all samples; the sgRNA-encoding regions were then amplified by PCR using HiSeq forward and reverse primers and sequenced on an lllumina HiSeq-4000 using HiSeq custom primer with previously described protocols at high coverage (Bassik et al., 2013; Kampmann et al., 2014). Primers used are summarized in Table 12.

For the individual sgRNA validation experiments, a similar protocol was used, except that CamES cells were cultured in basal medium seeded at 5,500 cells/cm⁷ after puromycin selection and transduced with a high MOI. Top 100 hits are summarized in Table 10.

Combinatorial sgRNA Library Construction

A library of 44 sgRNAs including a set of 19 genes was designed by using the top prediction hits from the single screens and six nontargeting negative-control sgRNAs. Any sgRNAs containing NsiI restriction sites, which were used for combinatorial sgRNA library construction, were excluded. Individual oligonuclotides encoding sgRNAs were synthesized in a 96-well format (Integrated DNA Technologieds), and cloned into pSLQ1373 individually as previously described. At the same time, the same sgRNA sequence was synthesized (Integrated DNA Technologies) using different forward sequence. These sgRNAs were cloned into pSLQ5004 individually as previously described. After sequencing validation, all pSLQ1373-sgRNA constructs were manually mixed and all pSLQ5004-sgRNA constructs separately mixed in equal amounts for combinatorial sgRNA library construction. To generate the pooled combinatorial sgRNA library, the sgRNA sequence were PCR amplified using two sgRNA forward and reverse primers from pooled pSLQ5004-sgRNA constructs, gel purified and ligated into the NsiO-digested pooled pSLQ1373-sgRNA constructs using In-Fusion cloning (Clontech). The combinatorial sgRNA-library pools were prepared in Stellar competent cells (TaKaRa) and purified with a Plasmid Maxi Kit (Qiagen). The representation of each of the double-sgRNA constructs was then quantified by NGS with the oligonucleotides listed in Table 11.

### High-Throughput Pooled Combinatorial Screens

The double neural differentiation screens were performed as two independent replicates. For both screens, 6 millions CamES cells were seeded at 40,000 cells/cm² density at day −1. Cells were transduced with pooled lentiviral double sgRNA library with an MOI of 0.3 at day 0 in basal medium supplemented with LIF and 2i. At day 1, puromycin was added at 1 μg/mL in basal medium with Doxycycline for another 24 hours. Fresh basal medium was changed with Doxycycline every day starting day 2. On day 12, cells were harvested and sorted for CD8+ and CD8− cells using Aria II cell sorter (BD Biosciences). Genomic DNA was harvested from all samples; the double sgRNA-encoding regions were then amplified by PCR using MiSeq forward and reverse primers and sequenced on an Illumina Miseq using HiSeq custom primer, which for the first sgRNA, and MiSeq custom primer, which for the second sgRNA. Primers used are summarized in Table 12.

For the individual double sgRNA validation experiments, a similar protocol was used, except that CamES cells were transduced with a high MOI.

### Primary Neurons Culture, Primary Astrocytes Culture and Induced Neurons Replating

Primary cultures of cortex neurons were prepared from postnatal day 1 wild-type black rat. Rats were decapitated, and their brains were removed in pre-cooled physiological saline. The cortex was dissected. Tissues were slightly minced and placed into a Papain Dissociation solution (Worthington Biochemical Corporation) containing 20 units/ml papain and 0.005% DNase in Earle's Balanced Salt Solution (Thermo Fisher Scientific). The solution was equilibrated in 95% O2, 5% CO2 before the tissue was incubated at 37° C. for 1 hour. After incubation, the tissue and solution mixture was triturated. Undissociated tissue was allowed to settle and the cloudy suspension was removed and centrifuged at 300×g for 5 minutes. The supernatant was then discarded and the cell pellet was resuspended in a DNase/albumin-inhibitor solution. A discontinuous density gradient was prepared by gently layering the cell suspension on top of an albumin-inhibitor solution in a centrifuged tube. The mixture was centrifuged at 145×g for 5 minutes. The supernatant was discarded and the neurons were resuspended in Neurobasal (Invitrogen) medium containing 2% B27 supplement, 2 mM glutamine and 0.5% penicillin/streptomycin. A total of 250,000 cells were plated onto a well of 24-well plates that had been pre-treated with 12.5 μg/ml poly-D-lysine (Sigma). The plates were incubated at 37° C. in a 5% CO2/95% air incubator and half of the medium was changed every three days.

Rat Primary Cortical Astrocytes (Thermo Fisher Scientific) were cultured and plated according to manufacturer's instructions. The astrocytes were fed every three days with fresh medium.

One week after culturing primary neurons and astrocytes, the induced neurons were gently removed from the dishes by trypsin dissociation and were replated onto primary neurons or astrocytes. Electrophysiological recordings were performed between day 14 and day 21 after replating.

### Generation of Induced Neurons

Preparation Before Induction
1. Embryonic skin-derived fibroblasts were isolated from E13.5 embryos of C57BL/6 mice as previously described (2010 nature, Vierbuchen et al.). Isolated fibroblasts were cultured and expanded in MEF media (Dulbecco's Modified Eagle Medium, Life Technologies) containing 10% Fetal Bovine Serum (Life Technologies), non-essential amino acids (Life Technologies), and sodium pyruvate (Life Technologies)) for 2 passages before use. Tail tip fibroblasts were isolated from the bottom third of tails from 4-day-old pups as previously described. Tail tip cells were expanded for 2 passages in MEF media before use.
2. Matrigel (growth factors reduced; BD Biosciences) was thawed on ice according to the manufacturer's instruction and dilute it in pre-cold PBS with a ratio of 1:30.
3. Diluted matrigel was added to 24-well plates. It was ensured that the quantity used was sufficient to cover the entire growth surface of the plates and keep the plates in 37° C. for 30 minutes to be ready to use.
4. Passage 1-2 MEFs were thawed and seeded into the matrigel-coated plates at a preferentially density of 25,000 cells per well of a 24-well plates. Cells were grown in the MEF medium for 4-5 days until confluent.

Induction of Induced Neurons
1. When MEFs were grown confluent, cells were infected with lentiviruses containing expression constructs of rtTA (driven by ubiquitin promoter) and additional lentiviruses overexpressing Asc11-Neurog1/Ezh2-Foxo1/Brn2/Nr4a1/Dmrt3/Jun/Suz12/Nr3c1/Tcf15/Zeb1/Mecom/Hoxc 8/Nr2f1 (driven by Tet-on promoter) in the presence of polybrene (8 mg/ml).
2. The next day, media was exchanged with basal medium (50% Neurobasal, 50% Dulbecco modified Eagle medium (DMEM)/Ham's nutrient mixture F12, 0.5% NEAA, 0.5% Sodium Pyruvate, 0.5% GlutaMax, 0.5% N2. 1% B27, 0.1 mM β-mercaptoethanol and 0.05 g/L bovine albumin fraction V; all from Thermo Fisher Scientific) containing doxycycline (2 mg/ml).
3. Culture medium was refreshed every 3-4 days during the induction period.

### Maturation of Induced Neurons

After lentiviruses infection for about 14 days (extensive neurites outgrowth should be observed in this stage), the induced cells were progressed for further maturation: Re-plate and co-culture directly with primary neurons/astrocytes.
1. Mouse primary postnatal cortical neurons or astrocytes were isolated and cultured for about 6 days before re-plating the induced cells.
2. The induced cells were dissociated by using 0.05% trypsin from the culture plate.
3. Cells were centrifuged for 3 min at 1000 rpm at room temperature.
4. The supernatant was discarded, fresh differentiation medium (basal media with addition of 200 μM ascorbic acid, 2 μM db-cAMP, 25 ng/ml BDNF, 25 ng/ml NT3, and 50 ng/ml GDNF) was added to gently re-suspend the cells and cells were re-plated to co-culture with pre-existing primary neurons/primary astrocytes.
5. Re-plated cells were co-cultured for about 14 days or longer (depending on the maturation process of the induced cells, which can be observed based on the development of the extensive neuritis outgrowth) to become functional mature. Half of the maturation medium was changed every 2-3 days.

### Flow Cytometry, Cell Surface Staining and Cell Sorting

The antibody CD8-APC was purchased from BD Biosciences. and Anti-PSA-NCAM-APC was from Miltenyi Biotec. Cells were harvested, washed, and adjusted to a concentration of 10⁶ cells/mL in ice cold PBS with 2% FBS. Cells were stained and incubated with diluted primary antibodies at 4° C. for 30 mins in Eppendorf tubes. After staining, cells were washed three times by centrifugation at 400 g for 5 mins and resuspended in 500 μL to 1 mL in ice cold PBS. Cells were kept in dark on ice and analyzed using BD Accuri C6 Cytometer. Cell sorting was performed by using Aria II cell sorter (BD Biosciences).

### Immunocytochemistry

Experiments were performed on cells seeded on plate (IBIDI) that had been coated with gelatin (0.1%) overnight at 37° C. Cells were washed twice with PBS, fixed in 4% Paraformaldehyde (Wako) for 15 mins at room temperature, permeabilized and blocked with 0.1% Triton X-100, 5% donkey serum in PBS (blocking buffer) for 1 h at room temperature. After three times wash with PBS, cells were incubated with primary antibodies. The following primary antibodies with indicated dilution in blocking buffer were used: Rabbit anti-Oct4 (Santa Cruz, 1:200), Mouse anti-Tuj1 (Covance, 1:1000), Rabbit anti-Map2 (Cell Signaling Technology, 1:200), Rabbit anti-NeuN (Abcam, 1:1000), Rabbit anti-vGluT1 (Synaptic Systems, 1:200), Rabbit anti-GFAP (Dako, 1:500), Rabbit anti-Olig-2 (Millipore, 1:500), Rabbit anti-Tbr1 (Abcam, 1:100), Rabbit anti-Synapsin I (Abcam, 1:200), Rabbit anti-GABA (Sigma, 1:250). Cells were incubated with primary antibodies at 4° C. for overnight, then washed three times with PBS. After staining with corresponding secondary antibodies in blocking buffer for 1 hour at room temperature, cells were washed three times with PBS and stained with DAPI (Vector Labs) for 5 mins. Washed cells were examined using a Nikon Spinning Disk Confocal microscope with TIRF.

### Efficiency Calculation

The following method was used to calculate the efficiency of neuronal induction. The total number of Map2+ cells with a neuronal morphology, defined as cells having a circular, three-dimensional appearance that extend a thin process at least three times longer than their cell body, were quantified 14 days after infection. The Map2+ and DAPI+ cells were counted from at least 20 randomly selected images at 20× magnification for each condition. The Map2+ cell number was divided by the number of DAPI+ cells to get a percentage of neuron-like cells.

### Electrophysiology

Lentivirus infections (with an additional sfGFP-expression virus) and transgene induction were performed similarly to as described for the fibroblast-induced neurons production, using basal medium. Patch-clamp electrophysiological recordings were performed on sfGFP positive fibroblast-induced neurons. GFP positive neurons located using a Lambda DG-4 illumination system and Q Imaging Fast 1394 Rolera-Mgi Plus camera controlled by Micro-Manager (Version 1.4) mounted on an Olympus BX51WI fluorescence microscope. Whole-cell responses were recorded using an MultiClamp 7008 (Molecular Devices) amplifier and headstage and low-pass filtered at 10 KHz before digitization using a DigiData 1440 data acquisition system (Molecular Devices). Data was stored on a PC running pClamp software (Version 10.4, Molecular Devices). Patch-pipettes were fabricated from 1.5 mm OD borosilicate capillary glass (Warner Instruments) using a microipette puller (Sutter Instrument, Model P-87) to give tip resistances of 2-4 MO. The series resistance for all recordings was under 10MΩ (Mean: 5.62MΩ, SEM: 0.38, n=12). Capacitance transients and series resistance errors were compensated for (70%) using the amplifier circuitry. The sodium and potassium currents currents were recorded in the voltage-clamp configuration at a holding potential of −80 mV. Spontaneous postsynaptic currents were recorded in the voltage-clamp configuration at a holding potential of −60 mV or −70 mV. Spontaneous action potentials were recorded in neurons held at −60 mV to −80 mV. Action potentials were also evoked by applying depolarizing current.

All experiments were performed at ambient room temperature (25° C.). The external solution contained (in mM): NaCl (130), HEPES-Na (10), KCl (5), CaCl2(2), Glucose (10). For voltage-gated sodium currents, tetraethylammonium (TEA, 5 mM) was added to the external solution and the internal solution contained (in mM): CsF (120), HEPES (10), EGTA (11), CaCl2 (1), MgCl2 (1), TEA-Cl (10), KOH (11). For voltage-gated potassium currents, tetrodotoxin (TTX, 500 nM) was added to the external solution and the internal solution contained (in mM): KF (120), HEPES (10), EGTA (11), CaCl2) (1), MgCl2 (1), KCl (10), KOH (11). For current clamp recordings of action potentials, 2 mM MgATP was added to the internal solution. All recording solutions had pH values of 7.3-7.4 with osmolality of 290-300 mOsm/kg. Drug applications were administered via local perfusion approximately 200 μm from the recorded cells at a flow rate of 0.2 ml/min and solutions were continually withdrawn from the recording chamber by vacuum aspiration. Drugs were applied until responses reached a steady-state level. Electrophysiological data were analyzed offline using Clampfit 10.4 and data was plotted using Graphpad Prism software.

Bloinformatic Analysis of sgRNA and Gene Hits

Data processing was conducted with custom scripts. Reads were mapped allowing for a mismatch for the first and last base pair of the spacer, which uniquely identified sgRNA. Each sample was normalized by the total read count. This gave a frequency for each sgRNA:

$${f}_{sgRNA} = \frac{sgRNA counts}{\sum sgRNA counts}$$

The paired Tuj1-hCD8+ and Tuj1-hCD8− were used to compute the enrichment scores. Specifically, frequencies as above were computed as above, and sgRNA with less than 1 count in the Tuj1-hCD8− library were discarded. Enrichment was computed for each sgRNA in each replicate as the log 2 fold-change from the Tuj1-hCD8− sample to the Tuj1-hCD8+ libraries. Enrichment was averaged across replicates and used as E_{sg} in subsequent analysis. For each gene, an enrichment score (ES_{gene}) was computed from the sgRNA enrichment above, as follows. An unnormalized enrichment score (E_{gene.top3}) was computed by averaging E_{sg} for the 3 sgRNA with highest E_{sg}. E_{gene.top3} was normalized by the distribution of nontargeting sgRNA as follows (Gilbert et al., 2014, supra).

Suppose a gene had N targeting sgRNA. 10000 bootstrap samples of size N were drawn from the nontargeting sgRNA. For each sample of size N, E_{sample.top3} was computed as above. This gave an empirical estimate of the distribution of E_{gene.top3} if the all the sgRNA targeting that gene had been negative control sgRNA. For the final, normalized gene enrichment score (ES_{gene}), the unnormalized enrichment score was divided by the 0.9 quantile of this empirical distribution:

$${ES}_{gene} = \frac{{E}_{gene , top 3}}{{quantile}_{samples} ( {E}_{sample , top 3} , 0.9 )}$$

After ranking genes by ES, the most enriched sgRNA was selected for each gene to subsequently validate.

### Bioinformatic Analysis of Double Screen

The count matrix was calculated by exact match for both ends, throwing all other reads out. The correlation of counts between replicates of the same condition was high (0.942-0.992), indicating high reproducibility of the double screen. Effect sizes for each gene pair was calculated using MAGeCK MLE (Li et al Genome Biology 2015, 16:281).

Suppose the null hypothesis that the guide pair of genes i and j have an effect size equal to the maximum of the individual effect size. This will be the case if one gene is the primary driver of neuronal differentiation. Note that the coefficients estimated by MACeCK (βᵢⱼ for genes i and j, in that order) arise from a generalized linear regression and should, if the model posited by MACeCK is correct, be normally distributed.

Consider the null hypothesis H₀: the effect of guide targeting two genes is less than the maximal effect of guides targeting either gene. The order of the guide is taken into account. A consistent but smaller effect is predicted with the order of the guides reversed. Let signm(x, y) be the function that returns the sign of the larger of the absolute values of the inputs. Under the null hypothesis βᵢⱼ=signm(βᵢ₀, β₀ⱼ) max(|βᵢ₀|, |β₀ⱼ|).

To this end, note that the standard deviation of βᵢⱼ is bounded above by

\sqrt{8_{β}_{ᵢ₀}²+8_{β}_{₀ⱼ}²}.

Therefore the difference βᵢⱼ−signm(βᵢ₀, β₀ⱼ) max(|βᵢ₀|, |β₀ⱼ|) has standard error bounded above by

$$\sqrt{{s}_{{β}_{ij}}^{2} + \sqrt{{s}_{{β}_{i 0}}^{2} + {s}_{β 0 j}^{2}}}$$

One can construct a test statistic to test H₀ as

$${t}_{i , j} = \frac{1}{2} \frac{{β}_{i , j} - signm ( {β}_{i 0} , {β}_{0 j} ) max ( \vert {β}_{i 0} \vert , \vert {β}_{0 j} \vert )}{\sqrt{{s}_{{β}_{i , j}}^{2} + \sqrt{{s}_{{β}_{i 0}}^{2} + {s}_{{β}_{0 j}}^{2}}}} + \frac{1}{2} \frac{{β}_{j , i} - signm ( {β}_{i 0} , {β}_{0 j} ) max ( \vert {β}_{j 0} , \vert {β}_{0 i} \vert )}{\sqrt{{s}_{{β}_{ji}}^{2} + \sqrt{{s}_{{β}_{j 0}}^{2} + {s}_{{β}_{0 i}}^{2}}}} .$$

The test statistic constructed does not have an exactly normal distribution due to the high correlation between estimates (since all gene-gene pairs are tested) and therefore an empirical Bayes approach is used to determine significant genes while appropriately controlling the false discovery rate (Efron Large-scale inference: empirical Bayes methods for estimation, testing, and prediction, volume 1. Cambridge University Press. 2012; Efron et al R package 2011).

### Determinants of CRiSPRa Guide Activity

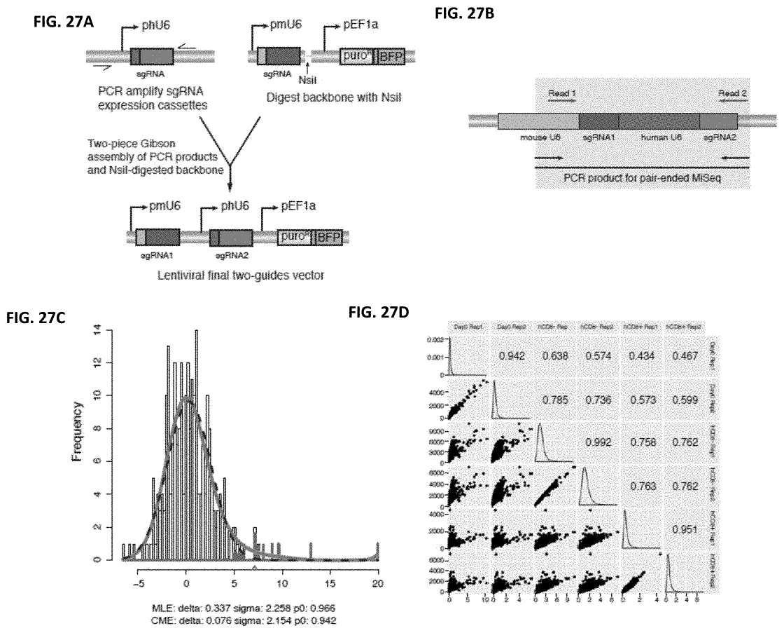

Since large variation gene effect size was observed (FIG. 27C) and an apparent mixture distribution in the top hits, a Bayesian hierarchical logistic regression mixture model was fit using stan (Carpenter et al 2017 J. of Stat. Software, Volume 76, Issue 1). Specifically, the following model was fit.
xᵢ=log₂ fold change of guide i;
gᵢ=gene associated with guide i;
xᵢ˜ZᵢN(μ_{g}_{ᵢ}, σ²)+(1−Zᵢ)N(0, 1.4²);
μ_{g}˜ N(3, 1.5²);
Zᵢ˜ Bernoulli(qᵢ);
yᵢⱼ=Indicator variable if guide i is in feature j;
gcᵢ=GC content of guide i;
dᵢ=distance from the TSS for guide i;

$${q}_{i} = logistic ( {β}_{0} + {\sum }_{j = 1}^{J} {β}_{j} {Y}_{ij} + {β}_{J + 1} {gc}_{i} + {β}_{J + 2} {d}_{i} )$$

βⱼ˜Laplace(0.2);
β₀˜N(0, 5).
   In this mixture model, features have a linear effect on the log-odds that the guides belong to the second component. In this way one can separate out gene-specific effects and compare guides targeting the same genes, but pooling the information across all genes. To shrink the feature effects towards zero, a Laplace prior is used. Eight chains were fit and good mixing in all chains and Rhat values near 1 was observed, indicating a good fit of the model.

TABLE 8

| Primers | sgRNA sequence |
|---|---|
| pSLQ1373- | gtatcccttggagaaccaccttgttgnnnnnnn |
| Forward | nnnnnnnnnnnnngttaagagctaagctggaaa |
| primer | cagca (SED ID NO: 386) |
| pSLQ1373- | gatcctagtactcgagaaaaaaagcaccgactc |
| Reverse | ggtgccac |
| primer | (SEQ ID NO: 387) |
| sgAscl1 | gaatggagagtttgcaaggag |
| | (SEQ ID NO: 401) |
| sgNeurog1-1 | ggctgctgggagttgtgcaa |
| | (SEQ ID NO: 405) |
| sgNeurog1-2 | gtgcactactgaatccaaga |
| | (SEQ ID NO: 530) |
| sgNeurog1-3 | gtcaatcagtagcaggcaaa |
| | (SEQ ID NO: 531) |
| sgMyod1 | ggtctccagagtggagtccg |
| | (SEQ ID NO: 406) |
| sgFoxo1-1 | ggttcaggatgagtggaggc |
| | (SEQ ID NO: 425) |
| sgFoxo1-2 | gaagacttcactcatcttgg |
| | (SEQ ID NO: 532) |
| sgFoxo1-3 | gtctcagcgatcggattgct |
| | (SEQ ID NO: 533) |
| sgNr2f1-1 | ggagccaagagaagggctgc |
| | (SEQ ID NO: 426) |
| sgNr2f1-2 | gaagtatatcatagtttcgg |
| | (SEQ ID NO: 534) |
| sgNr2f1-3 | gtttggagtttgagcatcct |
| | (SEQ ID NO: 535) |
| sgBrn2-1 | gaggaaggactgagaagact |
| | (SEQ ID NO: 428) |
| sgBrn2-2 | gtgtaagggatctttgttac |
| | (SEQ ID NO: 536) |
| sgBrn2-3 | gtgtttatgaaagtgtatgg |
| | (SEQ ID NO: 537) |
| sgEzh2-1 | ggttcctttcggcaccttgg |
| | (SEQ ID NO: 429) |
| sgEzh2-2 | gataactgaacagggagtgg |
| | (SEQ ID NO: 538) |
| sgEzh2-3 | gttcggccctctgattggac |
| | (SEQ ID NO: 539) |
| sgNr4a1-1 | gctaacgtgtagtctcgttg |
| | (SEQ ID NO: 431) |
| sgNr4a1-2 | gccacctaggagaagaagtg |
| | (SEQ ID NO: 540) |
| sgNr4a1-3 | ggtttcctttagcttagact |
| | (SEQ ID NO: 541) |
| sgDmrt3-1 | gaggagttgatagttgttcc |
| | (SEQ ID NO: 433) |
| sgDmrt3-2 | gttacaatagactttgaggc |
| | (SEQ ID NO: 542) |
| sgDmrt3-3 | ggcaggtattaatactcaag |
| | (SEQ ID NO: 543) |
| sgJun-1 | gagaataaagtgttgtgccg |
| | (SEQ ID NO: 435) |
| sgJun-2 | gtttacatccaggctttgag |
| | (SEQ ID NO: 544) |
| sgJun-3 | gtttggctgtctagtgacgg |
| | (SEQ ID NO: 545) |
| sgSuz12-1 | gaagctctcaaggcgagaaa |
| | (SEQ ID NO: 436) |
| sgSuz12-2 | gattctgtggaattgggttg |
| | (SEQ ID NO: 546) |
| sgSuz12-3 | gctcagtctcatctccactg |
| | (SEQ ID NO: 547) |
| sgNr3c1-1 | gtcactgctctttaccaaga |
| | (SEQ ID NO: 438) |
| sgNr3c1-2 | gttatggtttcaggctggaa |
| | (SEQ ID NO: 548) |
| sgNr3c2-3 | gactcttctgctcagtttgc |
| | (SEQ ID NO: 549) |
| sgTcf15-1 | gggatatgctcactttggga |
| | (SEQ ID NO: 439) |
| sgTcf15-2 | ggtcgtcgccttatagccgg |
| | (SEQ ID NO: 550) |
| sgTcf15-3 | gaagtgacaggatcagctat |
| | (SEQ ID NO: 551) |
| sgZeb1-1 | gaaggaactaagtttcttct |
| | (SEQ ID NO: 440) |
| sgZeb1-2 | gtgacaggtgatctaggcgc |
| | (SEQ ID NO: 552) |
| sgZeb1-3 | ggaaccttgttgctagggcc |
| | (SEQ ID NO: 553) |
| sgMecom-1 | gattctcaggcagggctcta |
| | (SEQ ID NO: 442) |
| sgMecom-2 | gaccagttcactgaaagatg |
| | (SEQ ID NO: 554) |
| sgMecom-3 | ggcagttctcttgcctagtg |
| | (SEQ ID NO: 555) |
| sgHoxc8-1 | gctctttcctctaacagccc |
| | (SEQ ID NO: 443) |
| sgHoxc8-2 | gaggtgagagttagtaagtc |
| | (SEQ ID NO: 556) |
| sgHoxc8-3 | gtcatcaaagaaagaatggc |
| | (SEQ ID NO: 557) |

TABLE 9

| | | SEQ |
|---|---|---|
| Gene | | ID |
| name | Primer sequence | NO: |
| RiboL7 F | accgcactgagattcggatg | 444 |
| RiboL7 R | gaaccttacgaacctttgggc | 445 |
| Ascl1 F | aagaagatgagcaaggtggagacg | 446 |
| Ascl1 R | gagatggtgggcgacagga | 447 |
| Brn2 F | tttcctcaaatgccctaagc | 448 |
| Brn2 R | ggaggggtcatccttttctc | 449 |
| Tuj1 F | agtcagcatgagggagatcg | 450 |
| Tuj1 R | agtcccctacatagttgccg | 451 |
| Map2 F | agcactgattgggaagcact | 452 |
| Map2 R | caattcaaggaagttgtaaagtagt | 453 |
| | gaagtttg | |
| Foxo1 F | gagtggatggtgaagagcgt | 490 |
| Foxo1 R | tgctgtgaagggacagattg | 491 |
| Nr2f1 F | ccaacaggaactgtcccatc | 492 |
| Nr2f1 R | attcttcctcgctgaaccg | 493 |
| Neurog1 F | cggcttcagaagacttcacc | 494 |
| Neurog1 R | ggcctagtggtatgggatga | 495 |
| Pou3f2 F | tttcctcaaatgccctaagc | 498 |
| Pou3f2 R | ggaggggtcatccttttctc | 499 |
| Ezh2 F | acttctgtgagctcattgcg | 500 |
| Ezh2 R | cgactgcattcagggtcttt | 501 |
| Nr4a1 F | gctagaaggactgcggagc | 504 |
| Nr4a1 R | attgagcttgaatacagggca | 505 |
| Dmrt3 F | agcgcagcttgctaaacc | 508 |
| Dmrt3 R | gcttttgacaacatctgggg | 509 |
| Jun F | gaaaagtagcccccaacctc | 512 |
| Jun R | aatcagacaggggacacagc | 513 |
| Suz12 F | tcgaaattccagaacaagca | 514 |
| Suz12 R | tgtggaagaaaccggtaaatg | 515 |
| Nr3c1 F | ggacaacctgacttccttgg | 518 |
| Nr3c1 R | ctggacggaggagaactcac | 519 |
| Tcf15 F | tctgcaccttctgtctcagc | 520 |
| Tcf15 R | aaccagggatccaggttcat | 521 |
| Zeb1 F | acagagaatggaatgtatgcatgtg | 522 |
| Zeb1 R | agattccacactcgtgaggc | 523 |
| Mecom F | acagcatgagatccaaaggc | 526 |
| Mecom R | ttatcccatctgcatcagca | 527 |
| Hoxc8 F | aaatcctccgccaacactaa | 528 |
| Hoxc8 R | tgtaagtttgtcgaccgctg | 529 |

TABLE 10

| | | Enrichment |
|---|---|---|
| Rank | Gene name | score |
| 1 | Foxo1 | 2.49122811 |
| 2 | Nr2f1 | 2.448600182 |
| 3 | Neurog1 | 2.43849068 |
| 4 | Rb1 | 2.435300527 |
| 5 | Pou3f2 | 2.385360453 |
| 6 | Ezh2 | 2.380072461 |
| 7 | Maz | 2.361103604 |
| 8 | Nr4a1 | 2.351837703 |
| 9 | Arnt | 2.317336958 |
| 10 | Dmrt3 | 2.304207908 |
| 11 | Sin3b | 2.280599668 |
| 12 | Jun | 2.277732884 |
| 13 | Suz12 | 2.276236754 |
| 14 | Klf12 | 2.269476929 |
| 15 | Nr3c1 | 2.249983644 |
| 16 | Tcf15 | 2.229200027 |
| 17 | Zeb1 | 2.221200461 |
| 18 | Nr6a1 | 2.208496165 |
| 19 | Mecom | 2.207944981 |
| 20 | Trim24 | 2.206262504 |
| 21 | Hoxc8 | 2.184103377 |
| 22 | Foxk1 | 2.171388615 |
| 23 | 2410080102Rik | 2.171161939 |
| 24 | Nr4a3 | 2.168779599 |
| 25 | Trp73 | 2.16579857 |
| 26 | Foxs1 | 2.162897697 |
| 27 | Ikzf3 | 2.15938851 |
| 28 | Nkx2-6 | 2.15063949 |
| 29 | Sox11 | 2.140964961 |
| 30 | 1110054M08.Rik | 2.139005342 |
| 31 | Crem | 2.133968618 |
| 32 | Meis3 | 2.131453549 |
| 33 | Bmyc | 2.130409666 |
| 34 | Epas1 | 2.129339686 |
| 35 | Nr2f6 | 2.128397081 |
| 36 | Nacc1 | 2.120269011 |
| 37 | Bsx | 2.120136772 |
| 38 | Foxd3 | 2.114601186 |
| 39 | Myog | 2.107435864 |
| 40 | Smad3 | 2.105254748 |
| 41 | Wt1 | 2.091731056 |
| 42 | Taz | 2.091306567 |
| 43 | Smad7 | 2.071136269 |
| 44 | Stra13 | 2.06971649 |
| 45 | Hoxc4 | 2.062634453 |
| 46 | Pou3f3 | 2.058607569 |
| 47 | Zbtb12 | 2.051837502 |
| 48 | Atf5 | 2.042025795 |
| 49 | Gtf2a2 | 2.041587014 |
| 50 | Pura | 2.040735147 |
| 51 | Snai1 | 2.040229657 |
| 52 | Ncor1 | 2.038396405 |
| 53 | Pcbp2 | 2.036271048 |
| 54 | E2f2 | 2.028758908 |
| 55 | Nfkbib | 2.023153101 |
| 56 | Gli2 | 2.021010016 |
| 57 | Nr0b1 | 2.020715359 |
| 58 | B230110C06Rik | 2.016733057 |
| 59 | T | 2.014396786 |
| 60 | Runx3 | 2.011724145 |
| 61 | Rxra | 2.011600497 |
| 62 | Mafk | 2.009964981 |
| 63 | Foxn1 | 2.006315586 |
| 64 | Smad4 | 1.999197443 |
| 65 | Meis2 | 1.998728368 |
| 66 | Hoxa1 | 1.996287157 |
| 67 | Zic1 | 1.992579239 |
| 68 | Sebox | 1.99248237 |
| 69 | Nfyc | 1.983084664 |
| 70 | Lmx1b | 1.980716237 |
| 71 | Lhx3 | 1.979175342 |
| 72 | Hmx2 | 1.978886945 |
| 73 | Arf6 | 1.977331424 |
| 74 | Nfatc3 | 1.975872129 |
| 75 | Neurod6 | 1.973516686 |
| 76 | Smarca4 | 1.972359038 |
| 77 | Twist1 | 1.971479015 |
| 78 | Gzf1 | 1.963483117 |
| 79 | Hoxc10 | 1.962998475 |
| 80 | Tbx4 | 1.962626034 |
| 81 | Npas2 | 1.962608209 |
| 82 | Ctbp1 | 1.960624385 |
| 83 | Gem2 | 1.960206991 |
| 84 | Is12 | 1.957324105 |
| 85 | Arid5a | 1.956887379 |
| 86 | Lef1 | 1.955552772 |
| 87 | RP24-399L6.2 | 1.953337042 |
| 88 | Smad5 | 1.949029539 |
| 89 | Lbx1 | 1.948838891 |
| 90 | Pax3 | 1.945680745 |
| 91 | Foxj1 | 1.944149198 |
| 92 | Tbx5 | 1.943975816 |
| 93 | Barh11 | 1.943598679 |
| 94 | Hoxd11 | 1.9410811 |
| 95 | Poulf1 | 1.939557398 |
| 96 | Klf3 | 1.938997548 |
| 97 | Pebp1 | 1.937292841 |
| 98 | Evx2 | 1.935442174 |
| 99 | Irx5 | 1.934100096 |
| 100 | Nkx6-3 | 1.928635054 |

TABLE 11

| | |
|---|---|
| pSLQ5004- | tggaaagccagaaacatgnnnnnnnnnnnnnnn |
| Forward | nnnnngttttagagctagaaatagcaagttaaa |
| primer | ataaggctagtcc |
| | (SEQ ID NO: 558) |
| pSLQ5004- | gatcctagtactcgaggtacctctaggc |
| Reverse | (SEQ ID NO: 559) |
| primer | |
| Two | accgtattaccgccagccttttgctcattaat |
| sgRNA | taaggtaccgagg |
| forward | (SEQ ID NO: 560) |
| primer | |
| Two | TGACGGGCACatgcatggtacctctaggctag |
| sgRNA | cgaattcAAAAAAAg |
| reverse | (SEQ ID NO: 561) |
| primer | |
| Scramble | GAACGACTAGTTAGGCGTGTA |
| sgRNA-1 | (SEQ ID NO: 562) |
| Scramble | GTTTAGTAGTTCGTCACACC |
| sgRNA-2 | (SEQ ID NO: 563) |
| Scramble | GCGACATGTCTGTTGGGCGA |
| sgRNA-3 | (SEQ ID NO: 564) |
| Scramble | GTATATAAGCCGGGCGCACG |
| sgRNA-4 | (SEQ ID NO: 565) |
| Scramble | GTCGAACCACGCGTTGATCG |
| sgRNA-5 | (SEQ ID NO: 566) |
| Scramble | GACCCATGACGGTCGACGGA |
| sgRNA-6 | (SEQ ID NO: 567) |
| sgFoxo1-1 | GGTTCAGGATGAGTGGAGGC |
| | (SEQ ID NO: 568) |
| sgFoxo1-2 | GAAGACTTCACTCATCTTGG |
| | (SEQ ID NO: 532) |
| sgNr2f1-1 | GGAGCCAAGAGAAGGGCTGC |
| | (SEQ ID NO: 426) |
| sgNr2f1-2 | GAAGTATATCATAGTTTCGG |
| | (SEQ ID NO: 534) |
| sgNeurog1-1 | GTGCACTACTGAATCCAAGA |
| | (SEQ ID NO: 405) |
| sgNeurog1-2 | GTGCACTACTGAATCCAAGA |
| | (SEQ ID NO: 530) |
| sgRb1-1 | GGCTACATACAGTCTAGGTT |
| | (SEQ ID NO: 427) |
| sgRb1-2 | GAGGAATCGAGAACTTAATT |
| | (SEQ ID NO: 569) |
| sgPou3f2-1 | GAGGAAGGACTGAGAAGACT |
| | (SEQ ID NO: 428) |
| sgPou3f2-2 | GTGTAAGGGATCTTTGTTAC |
| | (SEQ ID NO: 536) |
| sgEzh2-1 | GGTTCCTTTCGGCACCTTGG |
| | (SEQ ID NO: 429) |
| sgEzh2-2 | GATAACTGAACAGGGAGTGG |
| | (SEQ ID NO: 538) |
| sgMaz-1 | GGAAGGCATCTCTGGGAAGC |
| | (SEQ ID NO: 430) |
| sgMaz-2 | GCTCTGCAGGACACCCATGT |
| | (SEQ ID NO: 570) |
| sgNr4a1-1 | GCTAACGTGTAGTCTCGTTG |
| | (SEQ ID NO: 431) |
| sgNr4a1-2 | GCCACCTAGGAGAAGAAGTG |
| | (SEQ ID NO: 540) |
| sgDmrt3-1 | GAGGAGTTGATAGTTGTTCC |
| | (SEQ ID NO; 433) |
| sgDmrt3-2 | GTTACAATAGACTTTGAGGC |
| | (SEQ ID NO: 542) |
| sgSin3b-1 | GTGCAAGAATTCAGTCCACA |
| | (SEQ ID NO: 434) |
| sgSin3b-2 | GTGGTCAAGGTACACACCTA |
| | (SEQ ID NO: 571) |
| sgJun-1 | GAGAATAAAGTGTTGTGCCG |
| | (SEQ ID NO: 435) |
| sgJun-2 | GTTTACATCCAGGCTTTGAG |
| | (SEQ ID NO: 544) |
| sgSuz12-1 | GAAGCTCTCAAGGCGAGAAA |
| | (SEQ ID NO: 436) |
| sgSuz12-2 | GATTCTGTGGAATTGGGTTG |
| | (SEQ ID NO: 546) |
| sgKlf12-1 | GATTTGACCATCTCTTGCCG |
| | (SEQ ID NO: 437) |
| sgKlf12-2 | GAGTCACATTGATCCTGCAA |
| | (SEQ ID NO: 572) |
| sgNr3c1-1 | GTCACTGCTCTTTACCAAGA |
| | (SEQ ID NO: 438) |
| sgNr3c1-2 | GTTATGGTTTCAGGCTGGAA |
| | (SEQ ID NO: 548) |
| sgTcf15-1 | GGGATATGCTCACTTTGGGA |
| | (SEQ ID NO: 439) |
| sgTcf15-2 | GGTCGTCGCCTTATAGCCGG |
| | (SEQ ID NO: 550) |
| sgZeb1-1 | GAAGGAACTAAGTTTCTTCT |
| | (SEQ ID NO: 440) |
| sgZeb1-2 | GTGACAGGTGATCTAGGCGC |
| | (SEQ ID NO: 552) |
| sgNr6a1-1 | GATGACGGTCGGCCGTAGTT |
| | (SEQ ID NO: 441) |
| sgNr6a1-2 | GAATCAGGAAGGCTGTAGCA |
| | (SEQ ID NO: 573) |
| sgMecom-1 | GATTCTCAGGCAGGGCTCTA |
| | (SEQ ID NO: 442) |
| sgMecom-2 | GACCAGTTCACTGAAAGATG |
| | (SEQ ID NO: 554) |
| sgHoxc8-1 | GCTCTTTCCTCTAACAGCCC |
| | (SEQ ID NO: 443) |
| sgHoxc8-2 | GAGGTGAGAGTTAGTAAGTC |
| | (SEQ ID NO: 556) |
| sgOct4-1 | GTCTGGACAGGACAACCCTT |
| | (SEQ ID NO: 574) |
| sgOct4-2 | GAGTGCCTGTCTGCAAGGGA |
| | (SEQ ID NO: 575) |
| sgNanog-1 | GGAAGTTTCAGGTCAAGTGG |
| | (SEQ ID NO: 407) |
| sgNanog-2 | GCTGTAAGGTGACCCAGACT |
| | (SEQ ID NO: 576) |
| sgEsrrb-1 | GGTTAGTGGGCTCCAAGTGT |
| | (SEQ ID NO: 577) |
| sgEsrrb-2 | GGTGAGTGAGTGACACCCTC |
| | (SEQ ID NO: 578) |
| sgKlf2-1 | GAAAGGACCTGTGGACAGTT |
| | (SEQ ID NO: 579) |
| sgKlf2-2 | GCAAGAGGGTAATAGAGAGA |
| | (SEQ ID NO: 580) |

TABLE 12

| | |
|---|---|
| HiSeq | aatgatacggcgaccaccgagatctacacagat |
| forward | cggaagagcacacgtctgaactccagtcacnnn |
| primer | nnngcacaaaaggaaactcaccct |
| | (SEQ ID NO: 581) |
| HSeq | caagcagaagacggcatacgagatcgactcggt |
| reverse | gccactttttc |
| primer | (SEQ ID NO: 582) |
| HiSeq | gtgtgttttgagactataagtatcccttggaga |
| custom | accaccttgttg |
| primer | (SEQ ID NO: 583) |
| (the | |
| first | |
| sgRNA) | |
| MiSeq | aatgatacggcgaccaccgagatctacacagat |
| forward | cggaagagcacacgtctgaactccagtcacnnn |
| primer | nnngcacaaaaggaaactcaccct |
| | (SEQ ID NO: 581) |
| MiSeq | caagcagaagacggcatacgagatggtacctct |
| reverse | aggctagcgaattc |
| primer | (SEQ ID NO: 584) |
| MiSeq | ccactttttcaagttgataacggactagcctta |
| custom | ttttaacttgctatttctagctctaa |
| primer | (SEQ ID NO: 585) |
| (the | |
| second | |
| sgRNA) | |

### Results

### CRISPRa Screening Strategy for Neuronal-Fate-Inducers

This example describes the identification of novel TFs driving direct neuronal reprogramming from fibroblasts. Using primary fibroblasts as a screening platform is technically challenging. Firstly, as primary cells have limited expansion capacities, it is difficult to modify them to generate a homogenous population, which achieves consistent CRISPR activation activities. Secondly, the neuronal transdifferentiation of fibroblasts is inefficient and not well suited for the enrichment of the desired cell population for the subsequent sgRNA sequencing.

Thus, mouse ES cells were chosen as a screening platform for the generation of candidate TFs driving neuronal-fate. The ectopic expression of individual key TFs that are critical for neuronal transdifferentiation can also drive neuronal differentiation of mouse ES cells, which supports the use of mouse ES cell differentiation as a discovery tool for neuronal-inducing TFs. Besides, as a model of developmental biology, ES cells have been successfully used to elucidate roles of many master transdifferentiation TFs of other lineages. Finally, mouse ES cells are technically easy to be equipped with CRISRP activation tools and suitable for single sgRNA screens.

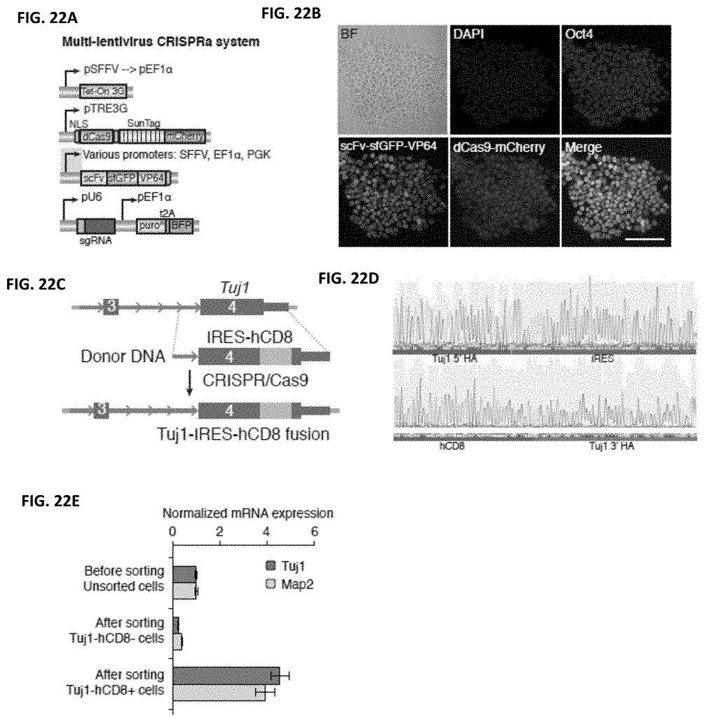

A polypeptide-based SunTag CRISPRa system in mouse ES cells (Tanenbaum et al., 2014, supra) was modified (FIG. 22A). After several rounds of optimization and clonal cell selection based on endogenous gene activation efficiency, a CRISPR-activating mouse ES (CamES) cell line containing lentivirus-transduced CRISPRa elements was generated (FIG. 22B). Next, the CamES cell line was modified with a neuronal reporter. The reporter CamES cell line carrying a biallelic human CD8 (hCD8) gene cassette appended downstream to endogenous Tuj1 via an IRES (internal ribosome entry site) (Tuj1-hCD8 CamES) (FIGS. 22C and 22D). The magnetic-activated cell sorting (MACS)-enriched differentiated hCDS+ cells expressed much higher neuronal markers (Tuj1 and Map2) than hCD8− cells (FIG. 22E), demonstrating that hCD8 expression is positively correlated with differentiated neuronal cells.

### An Unbiased Screen for Key Factors Promoting Neuronal Differentiation

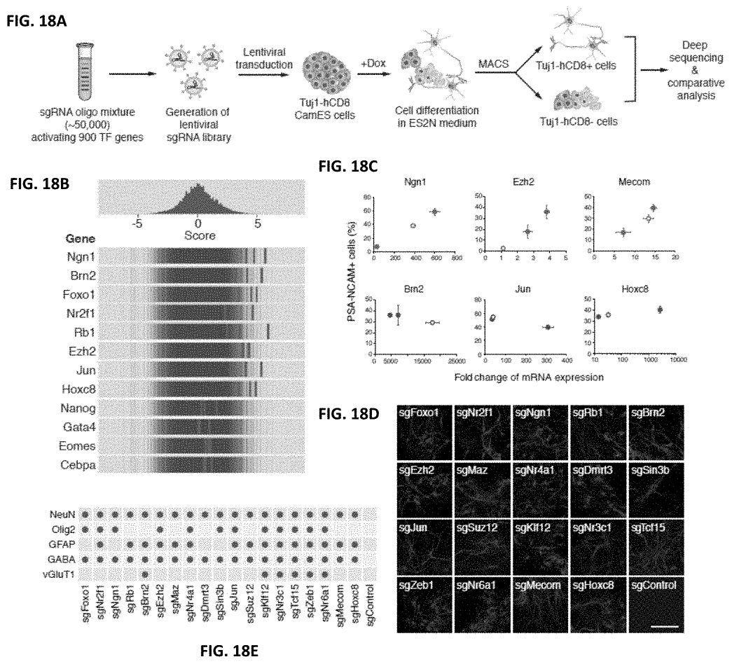

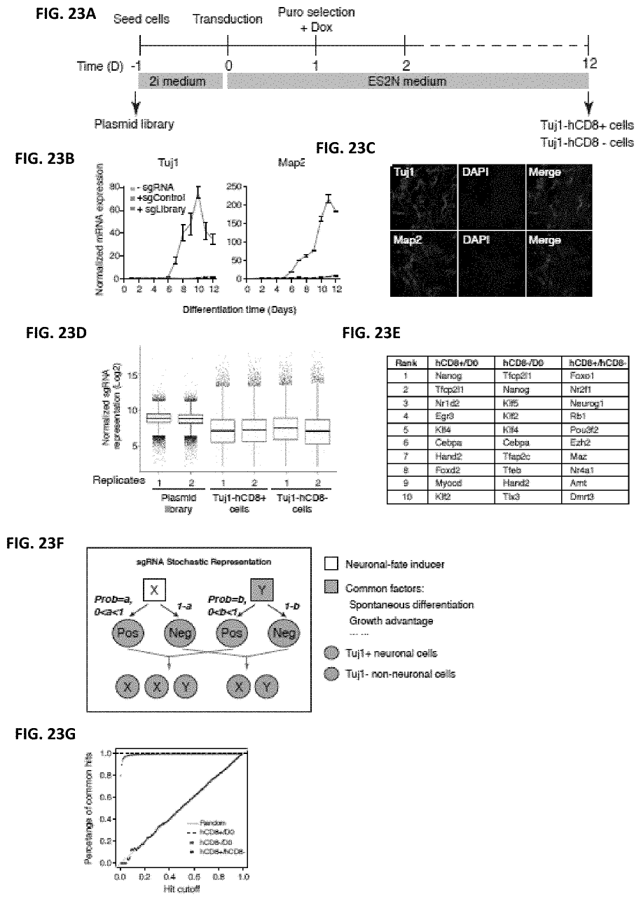

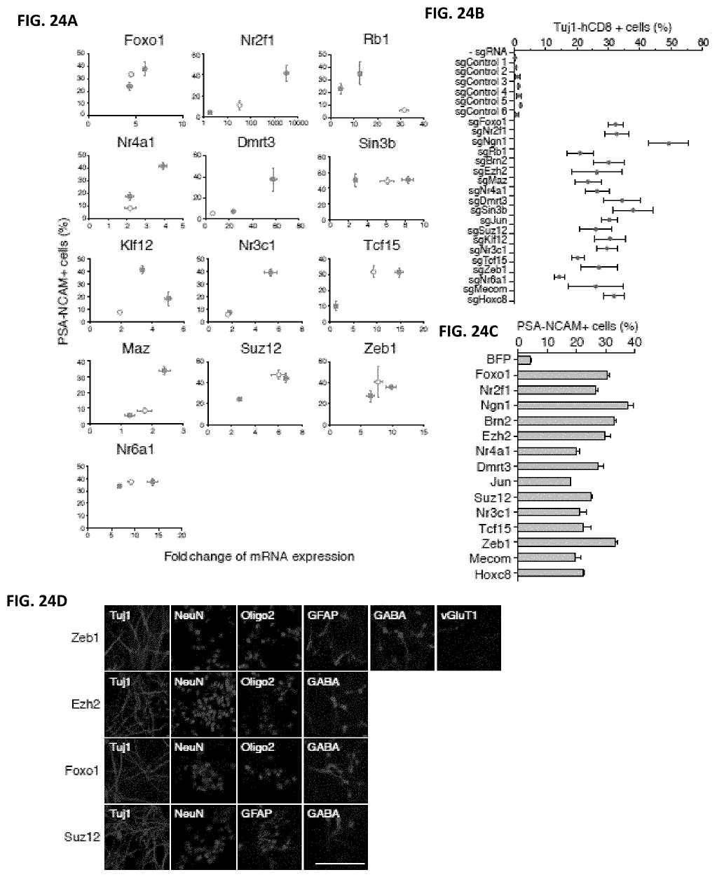

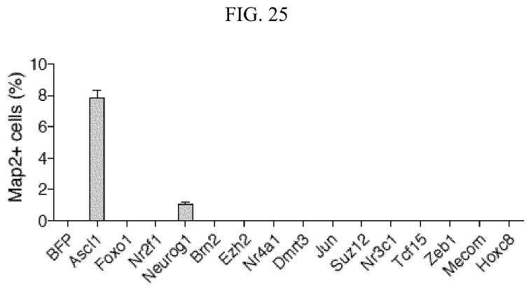

An sgRNA library targeting all putative TFs (˜800), with an average of 60 sgRNAs per gene was constructed. This sgRNA library also contained 9,296 non-targeting negative control sgRNAs, leading to a total of 55,336 sgRNAs (FIG. 18A). The sgRNA library was transduced into Tuj1-hCD8 CamES cells and 2i+Lif was removed from ES medium to allow neuronal differentiation (FIG. 23A). The Tuj1-hCD8 CamES cells showed highest neuronal marker expression between day 10 and 11 post-transduction (FIG. 23B). MACS were used to sort Tuj1-hCD8+ and Tuj1-hCD8− populations on day 12 (FIG. 23C), and the sgRNA distributions of these two samples were compared, as well as the plasmid library (FIG. 18A). The Tuj1-hCD8+ and Tuj1-hCD8− cell populations exhibited similar sgRNA depletion when compared to plasmid library (Figure S2D). A high correlation of enriched genes between the positive and negative Tuj1-hCD8 populations was found (FIG. 23E). The top hits relative to the plasmid pool in both populations contain many proliferation and self-renewal genes, but few are related to neuronal phenotypes (FIG. 23E). It was contemplated that this is because the predominant factors that determine sgRNA representation in both the Tuj1-hCD8+ and Tuj1-hCD8− populations are in common, such as the growth advantage of cells expressing proliferation and self-renewal genes, less proliferative capacity of desired neuronal cells, and the spontaneous differentiation (FIG. 23F). To control this bias and generate gene-level enrichment scores, sgRNA representation was normalized in Tuj1-hCD8+ samples to Tuj1-hCD8− samples, the enrichment of the top three guides for each gene was examined, and the empirical distribution of the non-targeting guides was used to normalize enrichment scores (FIGS. 18B, 25G, Table 10 and Experimental Procedures). Top-ranked genes (Table 10) were used to transduce individual sgRNAs to CamES cells and look for signs of neuronal differentiation. Among 20 sgRNAs tested, 19 efficiently induced neuronal differentiation, as measured by the expression of neuronal markers, NCAM, Tuj1 and Map2 (FIGS. 18C, 18D, 24A and 24B). A large fraction of validated genes has been previously characterized to act in early neural development. Examples included neuronal fate-inducing TFs such as Ngn1, Brn2, Klf12, Tcf15, and Mecom. These results were consistent with previous studies showing that the forced expression of these genes induce neuronal phenotypes of pluripotent cells. On the other hand, the function of the remaining hits varied considerably. Major categories included neuronal survival (Jun and Maz), cellular senescence (Sin3b and Rb1), homeostasis/metabolism (Foxo1, Nr4a1 and Nr3c1), and epigenetic regulations (Ezh2 and Suz12). In addition, the neuronal-inducing effects of the majority of hit genes were confirmed via the overexpression of their cDNA in unmodified mouse ES cells (FIG. 24C).

Cells expressing varied neuronal lineage markers resulted from the activation of different endogenous genes were detected (FIGS. 18E and 24D). For example, NeuN and GA BA expressing cells were found for all identified neuronal-fate-inducers. In addition, most hits also induced GFAP and Olig2 positive cells, which indicates the presence of astrocytes and oligodendrocytes. The Glutamatergic neuron marker vGluT1 expressed at varied levels across several hits, such as Zeb1, Brn2, and Nr6a1 (FIGS. 18E and 24D).

It was next tested if these neuronal factors induce transdifferentiation. As reported, Asc11 alone can induce neuronal transition from mouse fibroblasts. cDNAs of individual genes was transduced into mouse embryonic fibroblasts, cultured cells under transdifferentiation condition, and stained them with neuronal marker Map2. Among the 19 genes tested, only Ngn1 induced neuronal marker expression (FIG. 25). However, compared to Asc11, the transdifferentiation driven by Ngn1 was inefficient (7% vs 1% Map2+ cells). All of the other tested genes failed to induced Map2− cells.

### Neuronal-Fate-Inducing Activity of CRISPRa

To generate a deep view of how sgRNA design and gene activation level affects neuronal differentiation, other high-ranking sgRNAs of the 19 hit genes were investigated. Quantitative PCR results showed that effective endogenous gene activation (10 to 10,000 fold) was achieved by most of their cognate sgRNAs (FIGS. 18C and 24A). It was observed that, for the majority of hit genes, a higher gene expression level generally induced more efficient neuronal differentiation. Outliers of this trend included Jun, Brn2, Suz12, Tcf12, Zeb1, and Hoxc8. Cognate sgRNAs that induced higher expression levels of these genes generated similar amount of neuronal cells.

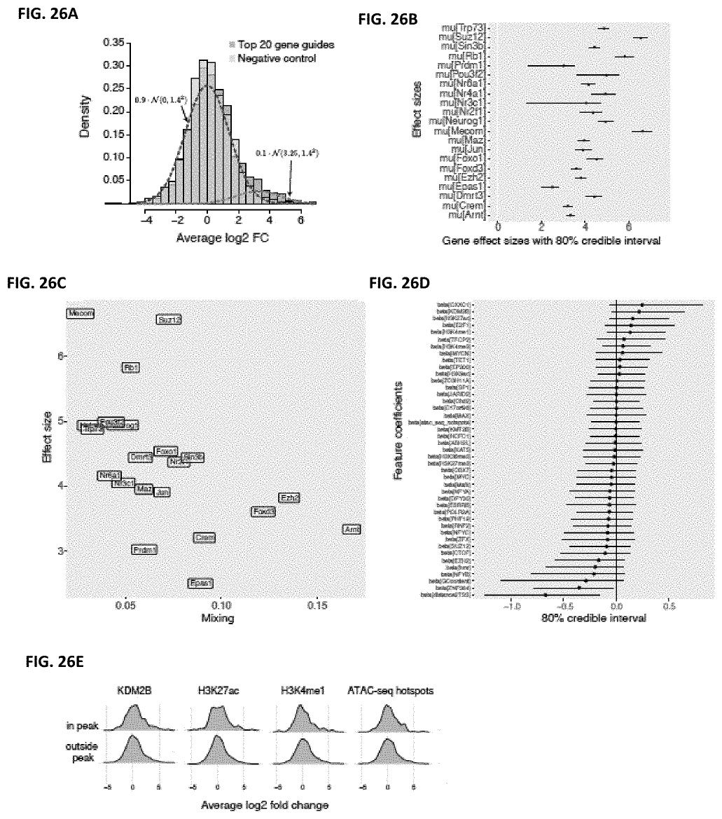

To investigate the determinants of CRISPRa activation in more depth, the targeting locations of top-ranked sgRNAs of the 19 hit genes was investigated. The observed signal followed a mixture distribution (FIG. 26A) (Horlbeck et al 2016 eLife 2016; 5:e19760). To determine what factors contribute to high neuronal signal, a hierarchical logistic regression mixture model was fit to estimate what genomic features can contribute to or prevent efficient activation (FIGS. 26B and 26C). It was found that KDM2B binding sites, H3K27ac peaks, and H3K4me1 peaks contribute to efficient activation (the top feature CXXC1 was primarily associated with a single gene, FIG. 26D). H3K27ac and H3K4me1 are known marks for areas of primed activation (Calo and Wysocka 2013 Mol Cell. 2013 Mar. 7:49(5):825-37), while KDM2B helps to maintain the stem cell state by recruitment of the polycomb repressive complex 1 (He et al. 2013 Nature Cell Biology 15, 373-384). Indeed, when controlling for other factors, being in a KDM2B increases the average observed log 2 fold change by nearly 1 (0.93, p=0.077, FIG. 26E). On the other hand, it was found that hotspots of open chromatin had little effect of guide efficiency (two-sided t-test, p=0.54, FIG. 26E). These indicated that the epigenetic features of sgRNA binding sites are important for CRISPRa activities.

### A Double-sgRNA Screen for Genetic Interactions Driving Neuronal-Fate

The strategy to use ES cells differentiation as a tool to discover lineage reprogramming factors was justified by the fact that Ngn1, a hit of the primary screen, is able to convert fibroblasts to neurons. However, as most hits failed to achieve transdifferentiation, the difference between the two processes was highlighted. Compared to ES cell differentiation, a direct lineage programming process utilizes profound transcriptional, epigenetic, and metabolic changes of target cells. These complex mechanisms tend to be initiated by synergistic genetic interactions, instead of a single factor. In most cases, direct lineage reprogramming can only be mediated by the ectopic expression of a gene cocktail. Thus, it was hypothesized that novel gene interactions greatly facilitate direct neuronal reprogramming.

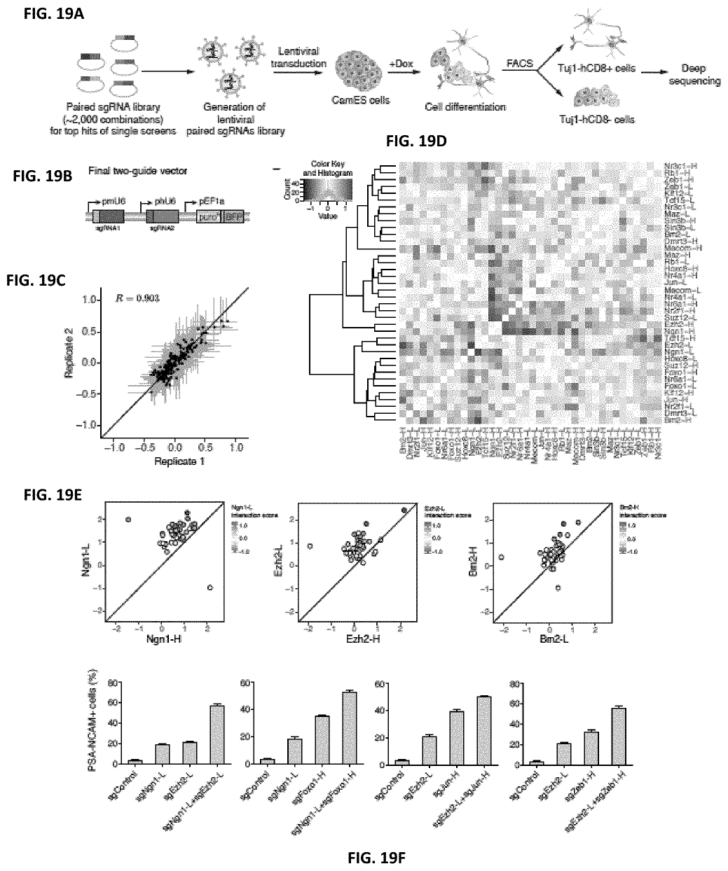

Current gain-of-function techniques, such as cDNA overexpression, are difficult to apply in a pairwise manner, even for a moderate number of genes. In addition, optimal gene expression levels are important for cell fate determinations. Overexpression libraries have limitations owing to dosage and functional issues, and thus may fail to cover genes' optimal expression level. To address these problems, a strategy to determine the gene interactions between the primary hits based on double sgRNA screen was developed. A library of dual-sgRNA-constructs targeting the top neuronal inducers was generated (FIG. 27A). For each hit gene, two sgRNAs were included. These sgRNA-High (H) and sgRNA-Low (L) were validated individually to drive different target gene activation levels (FIGS. 18C and 24A). The double sgRNA construction contains two sgRNAs driven by either human or mouse U6 promoter (FIG. 19B). Thus, two sgRNAs express independently. The library was generated through the ligation of two sgRNA elements, which can be easily scaled up (FIG. 27A). The library also included negative-control sgRNAs, i.e. non-targeting sgRNAs.

With the same strategy as in single CRISPRa screening, double CRISPRa screening was performed (FIGS. 19A and 27B). Pairwise interactions of sgRNAs were enriched relative to individual sgRNAs, and interaction scores were generated for each sgRNA pair (FIGS. 19D, 27B and 27D). It is noted that the correlations between two independent screening replicates are very high (FIGS. 19C and 27C), which indicates high reproducibility.

Hierarchical clustering of sgRNAs based on the correlation of their interactions shows that a fraction of sgRNAs tended to form a high number of interactions (FIG. 19D). These interaction-prone sgRNAs included many that drove low levels of neuronal differentiation compared to their counterparts. For example, Ngn1-H and Ezh2-H, which drove high gene activation and mediated efficient neuronal differentiation when applied individually, did not form strong interactions with other sgRNAs (FIG. 19E). On the contrary, their second top counterparts, Ngn1-1, and Ezh2-L, had synergistic effects with almost all other sgRNAs. A hypothesis to explain this is that in the screening system, some top sgRNAs already trigger saturated readout (neuronal differentiation), thus their interactions with other sgRNAs (even those synergistic) failed to be scored higher than themselves.

On the other hand, for genes whose higher activation lead to similar neuronal differentiation, such as Brn2 and Jun, a targeting sgRNA achieving highest activation tend to form stronger interactions then their counterparts (FIG. 19E). Foxo1-L and Foxo1-H, which mediated quite similar activation activities and differentiation efficiencies, both appeared as interaction-tendency hits (FIG. 19D). All together, these results showed that a library that covers a broad range of induced expression, including a “goldilocks” zone, is optimal for a gain-of-function double screen.

Gene Combinations Identified in Double CRISPRa Screen Convert Fibroblasts into Neurons

Based on false discovery rate, a list of gene pairs that showed strong synergistic effects was identified. Strong synergies included Ngn1+Ezh2, Ngn1+Foxo1, Tcf15+Zeb1, Tcf15+Foxo1, and Zeb1+Ezh2. To confirm these interactions, constructs expressing corresponding single and double sgRNAs were generated, and their effects in neuronal differentiation of CamES cells was tested. All of the identified sgRNA pairs showed additive effects in neuronal differentiation of mouse ES cells (FIG. 19F).

The synergistic links to Ngn1, the top hit in the single guide screen, that was identified have not been previously reported to drive neuronal transdifferentiation from fibroblasts. The ability of the above identified synergistic gene pairs to drive neuronal transdifferentiation from fibroblasts was investigated.

One gene pair, Ngn1+Ezh2, induced over 50% Map2+ cells, which is almost 50-fold more than Ngn1 alone (FIGS. 20A and 20B). Another double screening hit, Ngn1+Foxo1, induced nearly 45% neuronal cells. Zeb1+Ezh2, induced strong Map2 expression on neuronal cells (FIG. 20A). On the other hand, neither is able to mediate neuronal transdifferentiation alone. These results highlighted the power of double screen to discover strong synergies to mediate cell fate transitions.

Here, two new powerful neuronal inducing cocktails were identified: Ngn-1+Ezh2 and Ngn1+Foxo1. It was tested whether the induced cells possess neuron functions. The expression of other mature neuron markers in Ngn1+Ezb2 and Ngn1+Foxo1 induced cells, including Synapsin and NeuN was confirmed (FIGS. 20C and 28A). Furthermore, a large part of induced cells were Tbr1 positive, while a small part was GABA positive (FIGS. 20D and 28B). Moreover, these two combinations also induced neuronal transdifferentiation from tail tip fibroblasts with an extended culture time (FIGS. 20E and 28C).

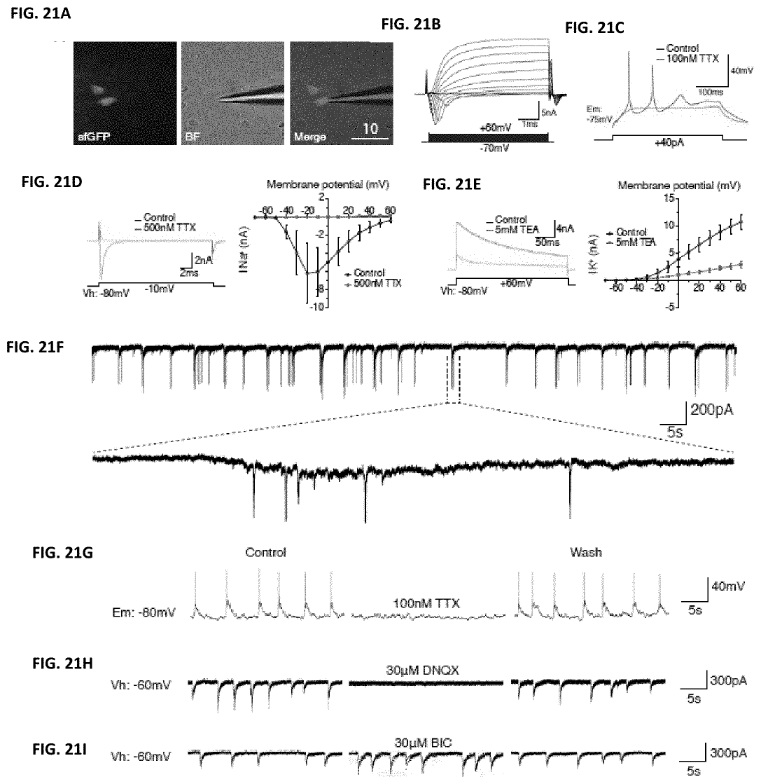

It was next assessed whether the induced neurons using Ngn1+Ezh2 and Ngn1+Foxo1 were capable of synaptically integrating into pre-existing neural networks. After 7 days' co-infection of cDNAs and a superfold GFP (sfGFP) reporter, the induced neuron cells were re-plated onto rat neonatal cortical neurons that had been cultured for 7 days in vitro. One week after re-plating, patch-clamp recordings from sfGFP-positive induced neuron cells were performed (FIG. 21A), In voltage-clamp mode, it was observed a fast activating and inactivating inward current followed by a slow activating and inactivating current (FIG. 21B). The action potentials could also be elicited by depolarizing the membrane held at −75 mV in current-clamp mode, which could be inhibited by the application of 100 nM tetrodotoxin (TTX), a selective blocker of voltage-gated sodium (Na+) channels (FIG. 21C). Inward currents could be blocked by the application of 500 nM TTX (FIGS. 21D and 28D), and outward currents could be inhibited by the application of 5 mM tetraethylammonium (TEA), a selective blocker of voltage-gated potassium channels (FIGS. 20E and 28E). Together the voltage-clamp studies show that these induced neurons express functional voltage-gated Na+ and K+ channels, which are critical in the ability of neurons to fire action potentials.

For all the induced neuron cells analyzed (5/5), action potentials that fired spontaneously were observed (FIG. 20F). Application of 100 nM TTX blocked the spontaneous action potentials, and washout of TTX completely reversed the blockade (FIGS. 21G and 28F). Spontaneous postsynaptic currents were recorded in induced neuron cells held at −60 mV in the voltage-clamp configuration. These currents could be blocked by application of 30 μM 6,7-dinitroquinoxaline-2,3-dione (DNQX), an AMPA and kainate receptor antagonist (FIGS. 20H and 28G). The blockade is reversible upon removal of DNQZ. On the contrary, the presence of 30 μM Bicuculline (BIC), a GABA_{A} receptor antagonist, slightly increased the observed frequency and amplitude of the spontaneous postsynaptic currents (FIGS. 20I and 28H). These experiments demonstrated the induced eurons were mostly glutamatergic excitatory neurons, which fired AMPA/kainate receptor-mediated spontaneous excitatory postsynaptic currents (EPSCs). The emergence of AMPA receptor mediated synaptic transmission is a key step in the development of mature glutamatergic synapses (Wu et al., Maturation of a central glutamatergic synapse. Science. 1996; 274:972), These data indicated that these induced neurons can form mature neurons as they form electrically active networks of cells in vitro. Overall these experiments demonstrate that functional synapses can be formed with induced neurons using Ngn1+Ezh2 or Ngn1+Foxo1.

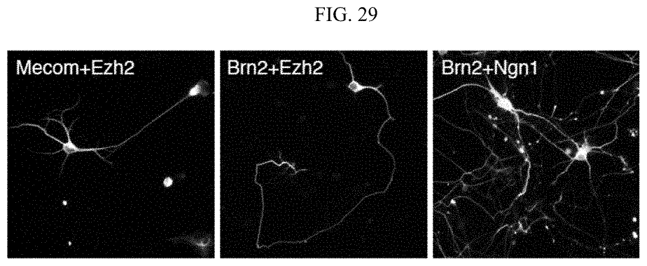

FIG. 29 shows three additional powerful neuronal inducing cocktails: Ngn1+Brn2, Brn2+Ezh2, and Mecom+Ezh2; which could drive neuronal transdifferentiation from fibroblasts.

Table 13 Shows Exemplary sgRNAs for Genes Targeted in Examples 1-4.

TABLE 13

| | | SEQ |
|---|---|---|
| gene | guide | ID |
| 6430411K18Rik | GTTGCTGGTTGATGAAGTTG | 586 |
| 6430411K18Rik | GCAGTTCCAAGTACCGGTGC | 587 |
| 6430411K18Rik | GGAAGGCAGCGCCATTCTGG | 588 |
| 6430411K18Rik | GACTCAGAGGACCCAAGAAA | 589 |
| 6430411K18Rik | GGGTCGAGTCCAGGATGAGT | 590 |
| 6430411K18Rik | GGTGGACTTGCTTGCAGGGT | 591 |
| 6430411K18Rik | GGATGATGAAGAAGAGGAGG | 592 |
| 6430411K18Rik | GCACACACTCCGACTCATCC | 593 |
| 6430411K18Rik | GGCTCCTTGGCACAGTACTC | 594 |
| Adipoq | GAACCTGGTTTAATCCAGCT | 595 |
| Adipoq | GGTAGAGAATGGCCAAAGCC | 596 |
| Adipoq | GTCCCATATAGGAACACTGC | 597 |
| Adipoq | GTTTCTAGAGAAATCACGTT | 598 |
| Adipoq | GCTGGGTCTGGTAGACACCC | 599 |
| Adipoq | GAAGCCAGAAGCCAGTAAGA | 600 |
| Adipoq | GTGAAGACCACGAGGCATTG | 601 |
| Adipoq | GTACAGGAAGGTTCCTGGTG | 602 |
| Adipoq | GGAGTCTTAAGGCAGCTGCC | 603 |
| Aebp1 | GATGTCACTTCCCTAGGCAT | 604 |
| Aebp1 | GGCACAGCGGGTTAGAGCAC | 605 |
| Aebp1 | GAGCACTCAAAGGGTCCAAG | 606 |
| Aebp1 | GAGGGATCACACAACAGCAC | 607 |
| Aebp1 | GTCATACTTGGACTGAATTT | 608 |
| Aebp1 | GAGTGGAGAGCTCTCCTCAC | 609 |
| Aebp1 | GGAATTCGAGCAGAGGAGCT | 610 |
| Aebp1 | GACAGAGAGGGTGAGGGTGA | 611 |
| Aebp1 | GGTGATCGCCAGTACCCTCG | 612 |
| Aebp1 | GGATGTCACTTCCCTAGGCA | 613 |
| Aes | GCCTGGACACCCAGGCTTCA | 614 |
| Aes | GAGGAAGCCTTAGAGACTGC | 615 |
| Aes | GATTCTGGTATCCCGGAGGC | 616 |
| Aes | GGCCTCTGATTCTGGTATCC | 617 |
| Aes | GAGTCTGTGGCCTTGGGACT | 618 |
| Aes | GTCTCTGTCTGTCTCAGGTA | 619 |
| Aes | GACACCTGTCCCACAGAGGT | 620 |
| Aes | GGATGGGACACCACTGAGGG | 621 |
| Aes | GACTCAGCAGCTTAAGAGGA | 622 |
| Ahr | GATGAGAAGGAAAGAAGCAC | 623 |
| Ahr | GCCCAAGCAGAAATGAGATC | 624 |
| Ahr | GTTGAGTGCCATGTAAGTTA | 625 |
| Ahr | GCCTTCCTTGTTGAAATAAC | 626 |
| Ahr | GCAGAGATGATAAAGGAAGA | 627 |
| Ahr | GGAAATGACAACAGGAAAGT | 628 |
| Ahr | GATTTAATGGGAGTGATGAG | 629 |
| Ahr | GTCATCACGTGCTGCGAAGA | 630 |
| Ahr | GTCCTTTAATAAGGTCTTCC | 631 |
| Ahr | GAATGTGTATGCCCTGTGAT | 632 |
| Ahrr | GGGAAGCTCCTGCTACCCAG | 633 |
| Ahrr | GTGTGAAATACCTTAAGAGT | 634 |
| Ahrr | GTCAGAACCTTGCATAGATG | 635 |
| Ahrr | GAGGCATCTGGAAGTGCAGA | 636 |
| Ahrr | GGATTTGGTGCACAAACTGG | 637 |
| Ahrr | GTGCCTAGGTGGAAGGTGGG | 638 |
| Ahrr | GGTGGGAGGGACTGGATGAG | 639 |
| Ahrr | GGGTAGCAGGAGCTTCCCAG | 640 |
| Aire | GTACAATCTCACTTTGCTGG | 641 |
| Aire | GCACCACGACACCCAAGGAA | 642 |
| Aire | GGGCCCAGCTTTCGAAAGCT | 643 |
| Aire | GAACAGGGAGCAAGGGACTG | 644 |
| Aire | GCTTGGAGGCCCTGTCTTTC | 645 |
| Aire | GAGATTCCTCACTGGCATGA | 646 |
| Aire | GTTTAGCCTAGAGCCAATCA | 647 |
| Aire | GGTCAGTCACTTCAGAGCCG | 648 |
| Aire | GCTAGAGACTGCCCTGCCTT | 649 |
| Alx1 | GCGGCTGTTAACCGGCTTGC | 650 |
| Alx1 | GGGCACAAGGCCAAGCAGAA | 651 |
| Alx1 | GCATCCGACAGCAAACGAGA | 652 |
| Alx1 | GACAGCAAACGAGAAGGCCA | 653 |
| Alx1 | GGGAGTCAGGGCTCTAAGAT | 654 |
| Alx1 | GTCGAGGCGACTACGATTCT | 655 |
| Alx1 | GCAGAACTGTTAAGTGAAGT | 656 |
| Alx1 | GTTGCTTGCTCCAcCTTCTC | 657 |
| Alx1 | GACAAATGCCAGGAGAGACA | 658 |
| Alx1 | GAAGCTTGAAATAACAGGCT | 659 |
| Alx3 | GAGAAGAGAGGCCTCTACTG | 660 |
| Alx3 | GAATGGAGAGTCTTGTAGGG | 661 |
| Alx3 | GCTGTAAATCAAGGCCAAAC | 662 |
| Alx3 | GACTGCAGGCTAGGCAGAGA | 663 |
| Alx3 | GGTTTCACAGTGGTCTGCCC | 664 |
| Alx3 | GAATGTTGGAGGAGGGATGG | 665 |
| Alx3 | GTCCTTGGTTGAGGGCAGTC | 666 |
| Alx3 | GCCATAACACTGTTTCTGAT | 667 |
| Alx3 | GCTTAAAGATCCCTTAGGTC | 668 |
| Alx4 | GTGAGGAGAATTCCAAAGAA | 669 |
| Alx4 | GTTAGCTTTGAGGTCTCCAT | 670 |
| Alx4 | GTTGAAGCAAAGGTCACCAA | 671 |
| Alx4 | GGATGAGAGGAGTGGGAAGA | 672 |
| Alx4 | GAAACCTGTGTCTGTCTCTC | 673 |
| Alx4 | GCTGGAGCAGATTGGAGGTA | 674 |
| Alx4 | GAGATAGGTGAGATTGGAGG | 675 |
| Alx4 | GATTCGACCCGGAGAAGCCT | 676 |
| Alx4 | GGAATTTCAACAGTGTGGTG | 677 |
| Alx4 | GTCAGCATCTGGATGCCTGA | 678 |
| Alyref | GTTCCCTAATGTCTAATTAC | 679 |
| Alyref | GACCAATCGCCGCTCGCTTC | 680 |
| Alyref | GAACTGCGGCATCTGCAGGA | 681 |
| Alyref | GAAGCGAGCGGCGATTGGTC | 682 |
| Alyref | GCCCAGCAAGCATGACAATA | 683 |
| Alyref | GGCACACGCCTCTAATCCCG | 684 |
| Alyref | GTCTTACCTCTGTAGCATCC | 685 |
| Amer2 | GTGTAGGGAAGGCTCCTTGC | 686 |
| Amer2 | GCGTTCTAAATCAACCTGAG | 687 |
| Amer2 | GACAAAGCAGCTTTCAGTGT | 688 |
| Amer2 | GCATTGTTCTTTGTGGACAT | 689 |
| Amer2 | GAAAGAGGAAGACTGAGCCC | 690 |
| Amer2 | GTGAGAGAGAGCAGTTTCCA | 691 |
| Amer2 | GTATTCTTTCTCCTCTGTGG | 692 |
| Amer2 | GCTAATTGGTATTTGACTGA | 693 |
| Amer2 | GAGAACAACCTGTGTGGGTA | 694 |
| Ap2b1 | GTTTCCCTGTCTCAGGGATA | 695 |
| Ap2b1 | GGGTGCGCGGGAGAACCAAA | 696 |
| Ap2b1 | GATCTCCAAACCTGATGGTC | 697 |
| Ap2b1 | GGCTACCTGGCAGTGAGGAA | 698 |
| Ap2b1 | GGGCTGGAGAGATGGCTCAG | 699 |
| Ap2b1 | GGTTTCCCTGTCTCAGGGAT | 700 |
| Ap2b1 | GGAGAGATGGCTCAGTGGGC | 701 |
| 4p2b1 | GGTCAAGATTTCCTGATTAA | 702 |
| Ap2b1 | GGCTGGAGAGGTGGCTCAGT | 703 |
| Ap2b1 | GATCAATCATGGTTAGCCAG | 704 |
| Ar | GCCTAGTCAGCTCCTGGAGA | 705 |
| Ar | GGCTTTAGAGAACGTAGTGC | 706 |
| Ar | GCACAGAGGTAAACTCCCTT | 707 |
| Ar | GAAACTTCACCGAAGAGGAA | 708 |
| Ar | GGGTCTACAACCTTTCTCTA | 709 |
| Ar | GAGTTAACTGAAACCTCAAG | 710 |
| Ar | GGAGTTAACTGAAACCTCAA | 711 |
| Ar | GCCCACCAGGACAAGCAGAA | 712 |
| Ar | GCGTCCCTTAAGCTTCTGTA | 713 |
| Arf2 | GCTGGTATGTGGGAGGAGCC | 714 |
| Arf2 | GACCAATGGAAATGGCAATA | 715 |
| Arf2 | GGGCTCTGGTAGGAGATTAC | 716 |
| Arf2 | GATTGGTCGTCTGTGGCTTC | 717 |
| Arf2 | GCAATGGTATTGAAGAGGCA | 718 |
| Arf2 | GCGCAAGAGTTCCCAGGAGG | 719 |
| Arf2 | GAGGCTTTGGGAGACTGCTA | 720 |
| Arf2 | GTAGGAGATTACTGGAACTC | 721 |
| Arf2 | GAATCTGGGTATTTCTGACC | 722 |
| Arf6 | GCTTCGTCGGCCCTTAGGAC | 723 |
| Arf6 | GAAGTCAGTGAAAGGGAGCA | 724 |
| Arf6 | GCTAGTTACTGAAGACGTTC | 725 |
| Arf6 | GGAACATGGCACCTGACCAG | 726 |
| Arf6 | GAGTTTAAACTTTCAAAGGC | 727 |
| Arf6 | GTCTGTTTCTTAAGAAATGC | 728 |
| Arf6 | GCAAGGGAAGGTGACAGAGG | 729 |
| Arf6 | GAGAGGCAGGTTGTAAGTGG | 730 |
| Arid3a | GCAGGGATATATTTAGCCAA | 731 |
| Arid3a | GGACCTGAGCACCACCTATG | 732 |
| Arid3a | GTCCTGGGAAAGCTTGGAAA | 733 |
| Arid3a | GAGGTGGTGGGTGTCTCTCC | 734 |
| Arid3a | GTCCCTTGTTAGACTGTTGT | 735 |
| Arid3a | GAACCGTGACGACCGTACCT | 736 |
| Arid3a | GCTCCTAGGTACGGTCGTCA | 737 |
| Arid3a | GGGCTTCAACCCAGCAGTGG | 738 |
| Arid3a | GGAGAAGAATGCTGGTGTGC | 739 |
| Arid3a | GTGACTTTCCGCTCAGAGGT | 740 |
| Arid3b | GCCTAGAGAACATTTATACT | 741 |
| Arid3b | GAGGAGGGACAGGCAGTAAG | 742 |
| Arid3b | GTTACATCTCTAGAGCAAGG | 743 |
| Arid3b | GAGACGCGGGCTAGTGAAGC | 744 |
| Arid3b | GTCCGTTGCTCTCGGTTTGG | 745 |
| Arid3b | GGTAAGGGAAATGGTCACCA | 746 |
| Arid3b | GCTTTCCTCAGCAAGGGAGA | 747 |
| Arid3b | GTTTGCCATGGTAGCACTTA | 748 |
| Arid3b | GAGGACCTGACCAGGGAAGT | 749 |
| Arid3b | GGCAGCGGCTTTCAGCAGAT | 750 |
| Arid5a | GTTCGCAGGTTGCCCGAGAC | 751 |
| Arid5a | GCTAGAGTCTTGGATCTCTT | 752 |
| Arid5a | GAACCGGCCAGGACCACTTC | 753 |
| Arid5a | GAAGTATGGTCACTGTCTCC | 754 |
| Arid5a | GAAATTGTCCCTTGGTGATC | 755 |
| Arid5a | GGATTAGCTGTGGCTTTGAA | 756 |
| Arid5a | GGCTGTGTCCCAGATCACCA | 757 |
| Arid5a | GTAGCTTGCCAAAGACTGGG | 758 |
| Arid5a | GCAGATCTCCATACCTAACC | 759 |
| Arid5a | GGATGGAGAGTGATGGAGGG | 760 |
| Arid5b | GTCTGCTCGGAATATGAATT | 761 |
| Arid5b | GTGATGTGCAGGTCATAATT | 762 |
| Arid5b | GTATATTATTCCTGTAGCGC | 763 |
| Arid5b | GCAAACCGCGCAATGCTCCA | 764 |
| Arid5b | GGCACCAATCTTTCCAGAGT | 765 |
| Arid5b | GATTGCATCAGGTCCTGGCA | 766 |
| Arid5b | GAGGTTTAATACACAATCCA | 767 |
| Arid5b | GAAATATTCAGAGCTGGGTT | 768 |
| Arid5b | GGCCTATCCGATACTGAGAA | 769 |
| Arnt | GTTTGAAACTCCAGGTTAAT | 770 |
| Arnt | GTGTAGTGGAGTCGTCTTTA | 771 |
| Arnt | GAAACAGTAAGTCGCCATAG | 772 |
| Arnt | GAGTTGGCTCTGAAGCTGGT | 773 |
| Arnt | GTGAGCCGACCAACTGGAGT | 774 |
| Arnt | GACTGACCGCGCCCATAGTT | 775 |
| Arnt | GGATTAGGGAAACAGCTGGT | 776 |
| Arnt | GCATTTCACTGACGTCAATT | 777 |
| Arnt | GGCCGGATTAGGGAAACAGC | 778 |
| Arnt | GCGTGTCTTCTGCCCAGGAT | 779 |
| Arntl | GAGAGATTCCTTCACAGAAC | 780 |
| Arntl | GACGAAGTGGCCTTGCTATC | 781 |
| Arntl | GAGGAGGAGGGAGAGCTGAG | 782 |
| Arntl | GGCTTCTCCTTGTGCAAACC | 783 |
| Arntl | GTGCCAATTGGTCCACTCCT | 784 |
| Arntl | GGTGCCAGTAGAAGATAAAC | 785 |
| Arntl | GTGGAGCTGICATTCCCGAT | 786 |
| Arntl | GGACCAATTGGCACGCTCTG | 787 |
| Arntl | GATAAATTCATTGTTCTGGA | 788 |
| Arntl2 | GGGAGCTTCATGTGCAGAGT | 789 |
| Arntl2 | GCAGCCTCACTTCCTGGCTC | 790 |
| Arntl2 | GCTGCTGGTGTCTGAGGAGT | 791 |
| Arntl2 | GACAACACCATTCAGTTGTT | 792 |
| Arntl2 | GGGTGTTCATTTATTTCTGG | 793 |
| Arntl2 | GCAGTCTGGAAGCTCAGGGT | 794 |
| Arntl2 | GGTCTGGAACCGGTTGGAGG | 795 |
| Arntl2 | GGAGGATGCTATTGATGGGT | 796 |
| Arntl2 | GGTGGGTGTTCATTTATTTC | 797 |
| Arntl2 | GGGATCGGTGAGAGAGCAAT | 798 |
| Arx | GAGGTCCATTGGTCCTAGAA | 799 |
| Arx | GGGAATGAGGGTGTCCATTC | 800 |
| Arx | GGCAGAGTGAATATTAAGTT | 801 |
| Arx | GAGTATTCAGAGAGGTGAAA | 802 |
| Arx | GGAGTCCTCAACGCAACTTG | 803 |
| Arx | GACCCAACTTCACTCAGGGT | 804 |
| Arx | GTCCTCAACGCAACTTGAGG | 805 |
| Arx | GAGGGAGGTGGGTAAGAGGT | 806 |
| Arx | GATGGTTGCCTCTGACACGT | 807 |
| Ascl1 | GTTGTTGCAGTGCGTGCGCC | 808 |
| Ascl1 | GTTCCCTAAGAAGCTGAGGC | 809 |
| Ascl1 | GAGGCAGGAGAATAAGTTGG | 810 |
| Ascl1 | GATGTTTGAGGATGACGTCA | 811 |
| Ascl1 | GAGGGAAAGGCTGCTCAGAC | 812 |
| Ascl1 | GGGCACAACTCGCTAAGGGT | 813 |
| Ascl1 | GCCTGAGACAGGGAGGGACA | 814 |
| Ascl1 | GCTAGACGCTATGGGAAAGG | 815 |
| Ascl1 | GAAGCAGAGACTGTGGAATG | 816 |
| Ascl2 | GCAGTGTGTATGGAGGTTGG | 817 |
| Ascl2 | GAGCATGTACTGCCAGTGTG | 818 |
| Ascl2 | GATTGTATTCTCTCAGGTCA | 819 |
| Ascl2 | GGTGACAGTTCCCTAGGGAT | 820 |
| Ascl2 | GGGAGGAAACAGGGCAGCAG | 821 |
| Ascl2 | GGAGCATGTACTGCCAGTGT | 822 |
| Ascl2 | GCTGGCTGTAAGGTGCAGAT | 823 |
| Ascl2 | GCACCTACCTAGTCCTTTGA | 824 |
| Ascl2 | GCCAGAAGGAAGCGTACGCC | 825 |
| Atf1 | GCTGGCCTGTTGTTCAGGCC | 826 |
| Atf1 | GTGGAAGTGCTGGATAAGAA | 827 |
| Atf1 | GAACTATCTGAGAGGATCCC | 828 |
| Atf1 | GTGACCTACAAAGTAAGGTC | 829 |
| Atf1 | GCTTGGAGATAGGCTGGCTG | 830 |
| Atf1 | GGACTTAGCATGTCCTTGTG | 831 |
| Atf1 | GATAGCGACTCCAGAGAGGT | 832 |
| Atf1 | GCCAAGGTCAGAGCATGGAT | 833 |
| Atf2 | GGCAACACCCATATTATCTC | 834 |
| Atf2 | GCTTCTCGGTACACGGAGAG | 835 |
| Atf2 | GACCGTTGTTTCGGTAACCA | 836 |
| Atf2 | GAAGAAAGCGGCAGGGATGC | 837 |
| Atf2 | GAGAGAAGAAAGGTGAGGTC | 838 |
| Atf2 | GGACCGTTGTTTCGGTAACC | 839 |
| Atf2 | GAGGGAATAGACCTGGTGTT | 840 |
| Atf2 | GTCAGCTGCTCATTACTGGT | 841 |
| Atf2 | GCCGACAATATCTAACCCAA | 842 |
| Atf2 | GGGAAGATCGGCTCCAGTTC | 843 |
| Atf3 | GAAGGAAGAGCCCTAAGGTC | 844 |
| Atf3 | GACAATCTCCCGCGTGAAGG | 845 |
| Atf3 | GACCGGAGCTGATCTGCATA | 846 |
| Atf3 | GGGATTACAGCAGCATCGCG | 847 |
| Atf3 | GGGTTGTGGAGGTGTGGAGC | 848 |
| Atf3 | GCGCAGGGATAAGAAAGGGC | 849 |
| Atf3 | GCCTTGGACTTGAGGAACCC | 850 |
| Atf3 | GGGTTTACCTGCCGGCAGGT | 851 |
| Atf3 | GATCTGCATACGGGCTCCCG | 852 |
| Atf3 | GGAGGGCAGAGGCCTGTGAA | 853 |
| Atf4 | GTGAGTCACTTAAACAGAAG | 854 |
| 4tf4 | GGAACTGACCCTATACAAAC | 855 |
| Atf4 | GGACTTGGCCTCAGAGACCA | 856 |
| Atf4 | GTCCCTGTCCCAGGACTGAC | 857 |
| Atf4 | GAGGCCTTGACCAGTACCTG | 858 |
| Atf4 | GGCAGTGAGGGCCTCTATGA | 859 |
| Atf4 | GCAGAGCCAATAGGAACTTG | 860 |
| Atf4 | GGAGGAGCCACCAGAGGTTC | 861 |
| Atf4 | GAGGAGCCACCAGAGGTTCC | 862 |
| Atf4 | GGCATAGGAGGTTAGACCTG | 863 |
| Atf5 | GGGCTTAACCCACGAGGTCT | 864 |
| Atf5 | GGACACACAGAACGATCATA | 865 |
| Atf5 | GACTTAACAAAGCCCATAGC | 866 |
| Atf5 | GGCCTCTAGGGACTTGCTAG | 867 |
| Atf5 | GCTACCTAGGAGCTGTTGCC | 868 |
| Atf5 | GAGGCCTCACAGGACAGGGT | 869 |
| Atf5 | GGAGTTGTGATCATCCCTGG | 870 |
| Atf5 | GAGAGAACAGCCTTGTGTGA | 871 |
| Atf5 | GTCCCTCTTGTCCTTCACCG | 872 |
| Atf5 | GCGCATGCGCAACAGGTTGT | 873 |
| Atoh1 | GCGGAACATTTCAACGGCAC | 874 |
| Atoh1 | GAATTTCCAGAACTGACTAG | 875 |
| Atoh1 | GACAACGTGAGAGCCTGGAA | 876 |
| Atoh1 | GCTTGGAGGGATCCCAGGCC | 877 |
| Atoh1 | GCTCAGATGAGACCCAGGGT | 878 |
| Atoh1 | GCAATCCCATGGACACGCTC | 879 |
| Atoh1 | GCCTGAGATCCCTCCAAGCC | 880 |
| Atoh1 | GAGAGCACTAGGAGCAAGCT | 881 |
| Atoh1 | GTTGAAATGTTCCGCTAGCA | 882 |
| Atox1 | GAGTGGTATCAGTTCCCTTT | 883 |
| Atox1 | GATGGCCAATTCCAGTTCAC | 884 |
| Atox1 | GGACACCAAAGCTGCGCTTC | 885 |
| Atox1 | GTCATTTCTGAAACAGGGCT | 886 |
| Atox1 | GGATTCACATCAGCTCCTTC | 887 |
| Atox1 | GTGGCTCTTGCATCAGCCTC | 888 |
| Atox1 | GCTAGGTTCCTCCCTTGGGC | 889 |
| Atox1 | GTTCTGGCTGGGAGGCTTCT | 890 |
| Atox1 | GTGCAGATTAAAGTCATGGC | 891 |
| Bach1 | GCGAGCTCCGTGTAACGTTG | 892 |
| Bach1 | GTAGCCCTGGCAGGACTTGT | 893 |
| Bach1 | GCCTTCAGCAGGTGAGGAAG | 894 |
| Bach1 | GCACTGTCTGTGTGTGTTTA | 895 |
| Bach1 | GCTTATTCCATGCTATTCTA | 896 |
| Bach1 | GCACCAGGTCACCACTTACA | 897 |
| Bach1 | GAAGCTAGTGATCATTCAGA | 898 |
| Bach1 | GCTCTTACTAGCGGAGGGCG | 899 |
| Bach1 | GTGGTTCTGACAGACATTCG | 900 |
| Bach1 | GTGGTCTACCAGGCTGTGAG | 901 |
| Bach2 | GAGGCAAAGACCGGAGCTCT | 902 |
| Bach2 | GGTTGTGTTAGTTGCTGTGC | 903 |
| Bach2 | GACTGAAATTGACCTCTACT | 904 |
| Bach2 | GAAGGCCAGTGTGGCACGTG | 905 |
| Bach2 | GTTTGTCCTTTGTTGCAATC | 906 |
| Bach2 | GGCTGGAGCAACACTTTGGA | 907 |
| Bach2 | GAGAAACACTAGAACCACTG | 908 |
| Bach2 | GCATGTGGCATTGCTAGCTT | 909 |
| Bach2 | GGGCCTGATTCAGCTTTCCA | 910 |
| Barhl1 | GTTGCAGCTACTTGGAGACC | 911 |
| Barhl1 | GGATCAGCCACTGCTAGTGC | 912 |
| Barhl1 | GGAGCTGCTAGGAACCCTTG | 913 |
| Barhl1 | GCCCTAGAGAGGCCACTGAC | 914 |
| Barhl1 | GGGTCCTAAGAGGTTGGGTT | 915 |
| Barhl1 | GGAAATTAGGAGGAAGAAAG | 916 |
| Barhl1 | GTTGAGCCACACACTCACCC | 917 |
| Barhl1 | GCACAGACCTAACTATTTAC | 918 |
| Barhl1 | GTTGCGCATCTGGGCAGCAG | 919 |
| Barhl1 | GAGAATTGTGGTGTTCTACA | 920 |
| Barhl2 | GAGCAGACATTTATTTATCA | 921 |
| Barhl2 | GTAGTCTTTGGCGCCAGAAC | 922 |
| Barhl2 | GCTGACGGCACAGCTTGTGC | 923 |
| Barhl2 | GAGTAGAGCTCAGACGTTGC | 924 |
| Barhl2 | GAGTGCTATCTAGATGGTCT | 925 |
| Barhl2 | GGATGACAAGCCAACGCGCG | 926 |
| Barhl2 | GACCAAGGCCTAACCTGGGA | 927 |
| Barhl2 | GTCGCGTTCCAGGTCCCAAA | 928 |
| Barhl2 | GCGCGTTGGCTTGTCATCCG | 929 |
| Barhl2 | GGGATGGAAGCATTGGAGGG | 930 |
| Barx1 | GGCGCACCTGTGGAATGGAG | 931 |
| Barx1 | GAAACTGGCCGCTCTGGGAG | 932 |
| Barx1 | GCTCTGTGGAGAGCATTCGT | 933 |
| Barx1 | GAGCTGTAGCAATGAGCTTT | 934 |
| Barx1 | GGAATGCCTGAACCAGTCCT | 935 |
| Barx1 | GCCTGATGTTGAGCCCTCCA | 936 |
| Barx1 | GCGCATAGTGTTCAAATACA | 937 |
| Barx1 | GCACCTGTGGAATGGAGTGG | 938 |
| Barx1 | GGGATCCAGTGCAATACACC | 939 |
| Barx1 | GTGAGTAGAAGCGGCTTTCT | 940 |
| Barx2 | GGCGCTAAGGGAGGACAGAG | 941 |
| Barx2 | GAAAGTTTGTAAGGCACCGA | 942 |
| Barx2 | GCTGTGGGCTGGGTTGAGAG | 943 |
| Barx2 | GGCAATCAAGTTTGCAACCT | 944 |
| Barx2 | GCTTGCGCACACCACTTCAG | 945 |
| Barx2 | GCATTTGTGAACCCGTACCC | 946 |
| Barx2 | GCGAGTACCAACGAGGGAAA | 947 |
| Barx2 | GCCATGAACACATGATTGTA | 948 |
| Barx2 | GTATAACACACTTCTGGTAT | 949 |
| Barx2 | GCTCCGAGCATGGTTAGCCG | 950 |
| Bbx | GTGAAAGACACATGGCAAAG | 951 |
| Bbx | GTCAGTGGGACCTGACCGTG | 952 |
| Bbx | GTCCAATTAGTGTTAATGTC | 953 |
| Bbx | GATCCCTTCTGCACTGAGTT | 954 |
| Bbx | GCCCATATCCACGTGGACTA | 955 |
| Bbx | GAGGCAGGGACAAACCAGGT | 956 |
| Bbx | GACTGGGACGTGAGAGCACA | 957 |
| Bbx | GAAAGGTAACAAATCAAACA | 958 |
| Bbx | GCTACTTAGTCTTT3AACTA | 959 |
| Bbx | GCTCAACACCTGGGAACTAA | 960 |
| Bcl6 | GTGGGAAGAGAGAGAGAGAA | 961 |
| Bcl6 | GTGGCTCGTTAAATCACAGA | 962 |
| Bcl6 | GCTCTGTTGATTCTTAGAAC | 963 |
| Bcl6 | GTCCTTTCTTCTCTCTTTAT | 964 |
| Bcl6 | GGTGGGAAGAGAGAGAGAGA | 965 |
| Bcl6 | GGTAGAGCCAGCCAGAGTGG | 966 |
| Bcl6b | GGCCTCTCCCTTCTGTTCTT | 967 |
| Bcl6b | GGTCGTATCGTGGATGGCTT | 968 |
| Bcl6b | GTCTGCATCCTTCCACGAGA | 969 |
| Bcl6b | GCGGAGGGTGGTAATATGGG | 970 |
| Bcl6b | GCTGAGAGCTTGATTGATGG | 971 |
| Bcl6b | GAGGTTAGTGGTGCGGAGGG | 972 |
| Bcl6b | GAAGCTAGGAGAGGATCTGA | 973 |
| Bcl6b | GCCGAAGAGAGCAGGGACCT | 974 |
| Bcl6b | GAGAAGGAGGAGTGATTGAC | 975 |
| Bcl6b | GCATGAGTACGCAACTAATT | 976 |
| Bhlhe41 | GCATTTAGCAGGAAGAAACG | 977 |
| Bhlhe41 | GGTTCCTCGAGTAGGACGAC | 978 |
| Bhlhe41 | GATAAGCCACGCCGAGAGTG | 979 |
| Bhlhe41 | GGCTTCCTCCAGTTCTTAAC | 980 |
| Bhlhe41 | GAGCAATTTACACCTTGAGC | 981 |
| Bhlhe41 | GGCGAGCCCACGTTTACTAC | 982 |
| Bhlhe41 | GAGACTCAAGTTTAAGGCAG | 983 |
| Bhlhe41 | GGAGGTGCCAGTAGTAAACG | 984 |
| Bhlhe41 | GGCTTATCGCACGAGGGAGA | 985 |
| Bhlhe41 | GGAGACAGGATTAAGGAGGG | 986 |
| Bmp7 | GGCACTTCCTCCTAAAGTCT | 987 |
| Bmp7 | GGCCAGGGACTCAGTACTGG | 988 |
| Bmp7 | GTGCTTCTGTGGTGGGAAGA | 989 |
| Bmp7 | GCGTGTTTGTTCTGTCACTT | 990 |
| Bmp7 | GACTGGAGCAAATGGAGTGT | 991 |
| Bmp7 | GTCCAGCACCCAAGGGATCC | 992 |
| Bmp7 | GTCTCTCTGTGGAGACTCAG | 993 |
| Bmp7 | GTTAGACAGTGAGGTACCAA | 994 |
| Bmp7 | GCACCGAAGAAGGGAGAGAT | 995 |
| Bmp7 | GGCAAGCATGGCAACCTCCA | 996 |
| Bmyc | GAACTGGGTCCAGATAGGAA | 997 |
| Bmyc | GGCATGATAGCCCAGGAGCT | 998 |
| Bmyc | GTTGGTCTGCTCACATGTTA | 999 |
| Bmyc | GTGCAAAGCCCAGTGGAGGC | 1000 |
| Bmyc | GAGCAGAATTCCAGCAGGAC | 1001 |
| Bmyc | GGTCCTGCCTCCAAGTGTCC | 1002 |
| Bmyc | GGACACTTGGAGGCAGGACC | 1003 |
| Bmyc | GATACTTTGAGCTTGGCGTC | 1004 |
| Bmyc | GAGCCTGGTGAAGGGACTGT | 1005 |
| Bnc1 | GGGTGCAGAAATATGTGGAG | 1006 |
| Bnc1 | GAAGCAGGTGCAACAAATTG | 1007 |
| Bnc1 | GCCTTCTGCCIGGTCCACAC | 1008 |
| Bnc1 | GGGACTGGCTGTTTGGGTCT | 1009 |
| Bnc1 | GGCTGTTTGGGTCTTGGGTC | 1010 |
| Bnc1 | GACCCAGAACAGGCACCTGG | 1011 |
| Bnc1 | GAGAGCAATGTGAAGGGCCA | 1012 |
| Bnc1 | GGTGACTTCGCTCTCAGAGT | 1013 |
| Bnc1 | GAAAGGACTCAGAGACAAGA | 1014 |
| Bnc1 | GTGCCTGTTCTGGGTCTACC | 1015 |
| Bptf | GCGAGAGGGAAGAAACAAGA | 1016 |
| Bptf | GGTTTGGGAGACACGCATTG | 1017 |
| Bptf | GAGGTGTGGAAGTTCGTAGC | 1018 |
| Bptf | GGCTCAACACAGGGTCCTCG | 1019 |
| Bptf | GTTGTTCCAGGAATGCACGC | 1020 |
| Bptf | GTCAGGTAGACATTATTTCC | 1021 |
| Bptf | GAGTGGTAAACTTACCCACA | 1022 |
| Bptf | GAACTTAAGGATAGGAAGGA | 1023 |
| Bptf | GCCTTTGGGAGCTCTCTATT | 1024 |
| Bsx | GAAGAGACTGAATGTCACTT | 1025 |
| Bsx | GTGGCTGGGAACCIAGACAT | 1026 |
| Bsx | GTTATGGGTAAGGGTGGCGG | 1027 |
| Bsx | GCGATTCCTCGAGCAATCTG | 1028 |
| Bsx | GTGTTCCTCTTTGTCTGGAA | 1029 |
| Bsx | GCCTGGCAGCAGCCAGTGAA | 1030 |
| Bsx | GTGAGGATCTACACAGTGGC | 1031 |
| Bsx | GAGGCAAGAAGACAAGCGCC | 1032 |
| Bsx | GGGAGCCCGGCGAACCAATA | 1033 |
| Carf | GGCCTACAAGATATCTCAGT | 1034 |
| Carf | GTCACTCAGAATAAAGAAGC | 1035 |
| Carf | GGTACTTGATGTGTGCGGGC | 1036 |
| Carf | GAACGAGTCGGAAGGGAACT | 1037 |
| Carf | GTAGTTGTAGATGAGGAATC | 1038 |
| Carf | GGAAAGGAGAACGAGTCGGA | 1039 |
| Carf | GCATAGTCCCATTTGTACCA | 1040 |
| Carf | GAGCTGAAGTGCTTGTGTCC | 1041 |
| Carf | GTCAACTGGACAATTAATGA | 1042 |
| Cav1 | GGTGCAAGGAAGAAGCACGG | 1043 |
| Cav1 | GGCACCTTGGAGGAATGGGC | 1044 |
| Cav1 | GCTCTGGAATCATAAAGATT | 1045 |
| Cav1 | GCCTCTTGGCTGTTCGCCAG | 1046 |
| Cav1 | GGCTTTCCCTCTGCTGGTTT | 1047 |
| Cav1 | GAGAAGGAATACAGAGGAGG | 1048 |
| Cav1 | GCCTCCTTTGTCTTATTGTA | 1049 |
| Cav1 | GGCGGTGGTACTTGTGAGGG | 1050 |
| Cav1 | GAGAGTGATCTAAGTAAGGG | 1051 |
| Cbfb | GGGAAAGGAGAAACAGAAGT | 1052 |
| Cbfb | GTAAGCATCTAACCAAATCA | 1053 |
| Cbfb | GCGCTAATTGTTTCTCATAT | 1054 |
| Cbfb | GCTGATACAGACCACTCAGT | 1055 |
| Cbfb | GGCTGATGCTAGCGTTTGCC | 1056 |
| Cbfb | GAACAGATTAGGTGCATGAA | 1057 |
| Cbfb | GAGGACTTTGCATACAGGGA | 1058 |
| Cbfb | GTGGACTGTGCACTGAAAGG | 1059 |
| Cbfb | GAAGTGCTGAGGAAGGAGCA | 1060 |
| Ccnt1 | GAGCTTCGTTTGAGTGTTTG | 1061 |
| Ccnt1 | GGATCTCCGGTAGAACGGAA | 1062 |
| Ccnt1 | GAGATGCTGATACAAGACTA | 1063 |
| Ccnt1 | GAACTACTGACCTGACGCGC | 1064 |
| Ccnt1 | GGTGGAGGAGAAAGGATCTC | 1065 |
| Ccnt1 | GACGTGACGAACTTCCTCCA | 1066 |
| Ccnt1 | GGTTCCTGGGTTCTTAGCTC | 1067 |
| Ccnt1 | GTAGACATTCTAAAGAGAAG | 1068 |
| Ccnt1 | GTGTGGCAAGGCACTGAGCA | 1069 |
| Ccnt1 | GTGTAGACATTCTAAAGAGA | 1070 |
| Cdkn1c | GGGAGTCGAGAAGGTGACTC | 1071 |
| Cdkn1c | GCTAACCAGGTCCAAGGTCG | 1072 |
| Cdkn1c | GCACACTGCTTCCAGAAGCA | 1073 |
| Cdkn1c | GAGGAACAGAATGAGGGCTG | 1074 |
| Cdkn1c | GTCTGTTGCGAGGAGGAAAC | 1075 |
| Cdkn1c | GAAGTACCCATTCTGCCCAA | 1076 |
| Cdkn1c | GGTCCAAGGTCGAGGTCCCA | 1077 |
| Cdkn1c | GGGCTCCTTTGTCTGCAGGC | 1078 |
| Cdkn1c | GGAGTGTGGTCCTGTGACCA | 1079 |
| Cdkn1c | GAGTTGGTTCGAAGAGCTGG | 1080 |
| Cdx1 | GATCCTCGTTGGTAATGGAA | 1081 |
| Cdx1 | GAGTTCTGCCCTTTCCTCTC | 1082 |
| Cdx1 | GCCAAGCTAGAGAATTCTTT | 1083 |
| Cdx1 | GGCGGTATGTCCACCCTTTG | 1084 |
| Cdx1 | GGTAGTGGCTTAGAGATGGA | 1085 |
| Cdx1 | GTAAGGTAGCGGGCGTCTCT | 1086 |
| Cdx1 | GGTTCCGTCTGTAAGGTAGC | 1087 |
| Cdx1 | GAAGGCCTAGCATGGAGGGC | 1088 |
| Cdx1 | GCCTGCCTGCCTGTCTTCAA | 1089 |
| Cdx1 | GACGCCCGCTACCTTACAGA | 1090 |
| Cdx2 | GAAATGATACTGACAGGAAC | 1091 |
| Cdx2 | GGGATGTGAAGGGTGGAAGG | 1092 |
| Cdx2 | GCAGTAATGAATAGCGACAA | 1093 |
| Cdx2 | GCATTCGGAAGACACAGGCT | 1094 |
| Cdx2 | GAGAGCATTGTCAGCATCCT | 1095 |
| Cdx2 | GAAGCTCGTAGCTAGCAAGA | 1096 |
| Cdx2 | GTCTTTGAACCTGTGATTGG | 1097 |
| Cdx2 | GGTGAGTACAGTAGCTCTGT | 1098 |
| Cdx2 | GAGCTTCCTCCTTCCAACCT | 1099 |
| Cdx2 | GCACTTTAACCTCCAATCAC | 1100 |
| Cdx4 | GTACATAGATGAGCAAGAGA | 1101 |
| Cdx4 | GGTCAATTACTCTTGAGTGT | 1102 |
| Cdx4 | GAAATGAGCAAGTGTCATTG | 1103 |
| Cdx4 | GAGCAGACTGCTCCTGCTCC | 1104 |
| Cdx4 | GAGTATGCGGCTCAGAGCAA | 1105 |
| Cdx4 | GAAAGGCAGGCCTCAGTGAA | 1106 |
| Cdx4 | GGTAGCCAGGTCACAACACA | 1107 |
| Cdx4 | GTCCCTGAAGTGGCGCTGAT | 1108 |
| Cdx4 | GCTGATGGGCTAGGAGCTAG | 1109 |
| Cdx4 | GTCATTGTGGTGGACCTGCA | 1110 |
| Cebpa | GAGAGACGTGGGTGCTCACC | 1111 |
| Cebpa | GCAGGTTTGTTTACCTGGGA | 1112 |
| Cebpa | GCTGGGTAGCAACGTCTGCC | 1113 |
| Cebpa | GTGAGCAGAGGATCGCTCTC | 1114 |
| Cebpa | GGTCACGGAACACGGACAAA | 1115 |
| Cebpa | GATCGAAGGCGCCAGTAGGA | 1116 |
| Cebpa | GTGACTTAGAGGCTTAAAGG | 1117 |
| Cebpa | GGAAAGTCACAGGAGAAGGC | 1118 |
| Cebpa | GTGCTAGTGGAGAGAGATCG | 1119 |
| Cebpa | GACTTRCCAAGGCGGTGAGT | 1120 |
| Cebpb | GGTCCCTGAACTGGCCTCTC | 1121 |
| Cebpb | GGAGAAAGTCTCCCAAGCCT | 1122 |
| Cebpb | GAAATGTTGGCAGGAAGCTA | 1123 |
| Cebpb | GCCTATTGAGCAAAGAACCT | 1124 |
| Cebpb | GTGGCCAGACCAACCAAGAA | 1125 |
| Cebpb | GCTCAGAGACAGCAGAGGGC | 1126 |
| Cebpb | GTCATTTCTCCAGCTCTTGG | 1127 |
| Cebpb | GGCTGCAAAGGTCTCTGGTG | 1128 |
| Cebpb | GGGTTCTGCCACACTGTGTC | 1129 |
| Cebpb | GATCTGTTTCCCAAGAGTTG | 1130 |
| Cebpd | GTTGTGTTTACAAGACAGCG | 1131 |
| Cebpd | GAACCACGGTTCACTAGTTC | 1132 |
| Cebpd | GATGCTATGCTACCACCAGG | 1133 |
| Cebpd | GAAACGCACCGCGGTTAGGG | 1134 |
| Cebpd | GCTCCTACCTTCAGTTCCTG | 1135 |
| Cebpd | GCCTTCAGACATAGCAAAGG | 1136 |
| Cebpd | GACATAGCAAAGGCGGAACA | 1137 |
| Cebpd | GTCCTGCTTTGCGCGTGTCG | 1138 |
| Cebpd | GACGCCTTCAGACATAGCAA | 1139 |
| Cebpd | GTTGCTGAACCTAACCTCGA | 1140 |
| Cebpe | GTTTAGACCAAGTTGGCACT | 1141 |
| Cebpe | GCAGCTACCAGCTTCTcCTT | 1142 |
| Cebpe | GGTAGGTGGAGTTCAGGACT | 1143 |
| Cebpe | GAAGCCTTCCCTAGCCCAGC | 1144 |
| Cebpe | GGAAGCCTTCCCTAGCCCAG | 1145 |
| Cebpe | GTAGATAGGGAAGCAGGAGA | 1146 |
| Cebpe | GGGCTGCCAGGACATAGCTG | 1147 |
| Cebpe | GATCCTTTCTGTTGGTTCTG | 1148 |
| Cebpe | GAAACTGGTCCCGCTGGGCT | 1149 |
| Cebpe | GGGTTAGTAGAAGATCAAGA | 1150 |
| Cebpg | GAGCTATTCATATGAAGTAT | 1151 |
| Cebpg | GAACTGTTCCCGGGAGACCC | 1152 |
| Cebpg | GACTCCTGGGCATTGACTGC | 1153 |
| Cebpg | GCCACCACCGACAGCCTAAG | 1154 |
| Cebpg | GGATTCCTCGAAGTCTTATG | 1155 |
| Cebpg | GCTTCTATTGGTCACGGCGG | 1156 |
| Cebpg | GCATGATGCAGATCTGTGAA | 1157 |
| Cebpg | GATAGAACTTTGCTTGCCAT | 1158 |
| Cebpg | GGAGGACCACAGTGTGACTG | 1159 |
| Cebpg | GAGGGTGTTCCTAGAATAGA | 1160 |
| Cebpz | GTGACGCACTTCCTATTGCG | 1161 |
| Cebpz | GTATGTCCAATGACCTATAT | 1162 |
| Cebpz | GCTCCTCTGTGTACACACAC | 1163 |
| Cebpz | GATTGCTTATTTGTGCCATG | 1164 |
| Cebpz | GGTGGAACTTGGCCCTGGTC | 1165 |
| Cebpz | GCCTTCCCTGTATTTGGAGA | 1166 |
| Cebpz | GGCGGTGGCTCAACACCTGA | 1167 |
| Cebpz | GTGTACACAGAGGAGCCCGA | 1168 |
| Cebpz | GCCGCGCCATACGGTTTCCA | 1169 |
| Cebpz | GAAGCTCACTCTCAGGGTGA | 1170 |
| Clock | GAACCTAAGCGAGCAGCAGA | 1171 |
| Clock | GCAGAAACTGTGCCTTTCGA | 1172 |
| Clock | GGGTCGTCCAGGTCCATCTC | 1173 |
| Clock | GGACAGAGTGGAGAATGGGT | 1174 |
| Clock | GCACATGGTGTTTAAGGCCA | 1175 |
| C1ock | GTGGTCCAGGCAGGACACTG | 1176 |
| Clock | GACAATGAAACCATTAAAGG | 1177 |
| Clock | GAGGACAATGAAACCATTAA | 1178 |
| Cnot3 | GACTTAAGAAGGTGAAACCT | 1179 |
| Cnot3 | GTACGTCGCTCTGCGCCGTT | 1180 |
| Cnot3 | GAACTGCTTCTAGCTCTATC | 1181 |
| Cnot3 | GAAGCTTATCTAGTGGGAGA | 1182 |
| Cnot3 | GATCAGATAACAGCCTAGAC | 1183 |
| Cnot3 | GAATTTCCATGGATCATTTC | 1184 |
| Cnot3 | GCGTGGGACTGACGTTTCTC | 1185 |
| Cnot3 | GTTTGCAACCTAGTCAGCAA | 1186 |
| Cnot3 | GCATAGCGTGTGAGTGTTAA | 1187 |
| Cnot3 | GACTGAGAAACACAAGGCGT | 1188 |
| Creb1 | GAAACATGCTACAAGAAGAA | 1189 |
| Creb1 | GCACGATCCGAGCCTCACTG | 1190 |
| Creb1 | GCTAAGAACCGTGGGAGGAA | 1191 |
| Creb1 | GCTATGGCACAGGTGGCATG | 1192 |
| Creb1 | GAGCAGTTGCGGTAGCTTTG | 1193 |
| Creb1 | GGCTCAGATGACTCCTGCAC | 1194 |
| Creb1 | GGAACTTTGACGCGCCGCGA | 1195 |
| Creb1 | GGTTTGTGTGTAGCCAGATT | 1196 |
| Creb3l2 | GCCGGAGCTGGTTCTTTGCT | 1197 |
| Creb3l2 | GAGCGTCGCAATGGACCAAT | 1198 |
| Creb3l2 | GTCACTGGCCTGGAAGGAGG | 1199 |
| Creb3l2 | GAAACATAGATCAATGAGCT | 1200 |
| Creb3l2 | GGGCAGAGCTCAAGAGCCCA | 1201 |
| Creb3l2 | GGTCAGGTCAATATAGAAGG | 1202 |
| Creb3l2 | GAGGAGACTGAAGAAATCCA | 1203 |
| Creb312 | GAGGGAACCCAGGTCACAGA | 1204 |
| Creb3l2 | GAAGAGGCTAGTGTGGTCCA | 1205 |
| Creb3l2 | GGGAGGGACATGGATGAGAA | 1206 |
| Crebbp | GTGTGGCACACCCAAGTGAG | 1207 |
| Crebbp | GGAAGTCCCTCTAACACTTT | 1208 |
| Crebbp | GTTCAGAGGCCTCCGAATTG | 1209 |
| Crebbp | GACCAGCATCACTGCATCTG | 1210 |
| Crebbp | GCCATTACTAGCATAGGGCG | 1211 |
| Crebbp | GTTGTCTACTAGTCTGTCCC | 1212 |
| Crebbp | GTGAATGTAGGATGCTGGTG | 1213 |
| Crebbp | GGGCCCATCTCAGATCCAGG | 1214 |
| Crebbp | GTTTACCAACAGTATCCTTT | 1215 |
| Crem | GTTAGCTACAGTACTACAGA | 1216 |
| Crem | GAAAGAAAGATTGGAATTCA | 1217 |
| Crem | GGACCAGACTCTCTTCAGGA | 1218 |
| Crem | GCAAGTGAAGATTAAAGATG | 1219 |
| Crem | GCAAATAGAACTTAGCATTG | 1220 |
| Crem | GAACAACCATTTGTGAGTTT | 1221 |
| Crem | GAAGGTTACAAATAGGCCAG | 1222 |
| Crem | GCAGCCTCTTGGCTACTAAC | 1223 |
| Crem | GACCTCAATCCCAAAGTGTG | 1224 |
| Crx | GGTGTCACTGGGAAGCATGG | 1225 |
| Crx | GTTCTGCTTCTCTAAACACC | 1226 |
| Crx | GGGTGGTGGGATTAAGCAGA | 1227 |
| Crx | GAAGGCTAAACTATGCAGAC | 1228 |
| Crx | GAAACAATCCTTCAGGCCAG | 1229 |
| Crx | GTCACTGGGAAGCATGGAGG | 1230 |
| Crx | GATCTGGAAGGGTAATCCCA | 1231 |
| Crx | GCTCTCTGAAGCTTGACAGG | 1232 |
| Crx | GGCCCTAATCTCTCCTAGCA | 1233 |
| Crx | GCCCTAATCTCTCCTAGCAG | 1234 |
| Crygf | GGCAACAGAGGTGAATTGCC | 1235 |
| Crygf | GTAGAGAGAAGAAACCTCCT | 1236 |
| Crygf | GAAAGAATGGAAGGCAGGGA | 1237 |
| Crygf | GGATAAGTCTGTCAGATTCA | 1238 |
| Crygf | GAGATCATGATGAGTGTATG | 1239 |
| Crygf | GCAGGAAGAGGTGGAAGGCA | 1240 |
| Crygf | GAATCTGACAGACTTATCCC | 1241 |
| Ctbp1 | GGTCTCTTTGGTTGGGTACA | 1242 |
| Ctbp1 | GAAGGATGCTGAAGGCCATA | 1243 |
| Ctbp1 | GTGTCCCAGAAGTTGAGGGA | 1244 |
| Ctbp1 | GGAAAGTACAGCTTTGCCAG | 1245 |
| Ctbp1 | GCAGGGACCATCCCTGGAGT | 1246 |
| Ctbp1 | GTAGAGATGTGGAGATGCAC | 1247 |
| Ctbp1 | GGTTTCCTGGGAGGCCCTAA | 1248 |
| Ctbp1 | GCCTCAGCAGATATGTAGGT | 1249 |
| Ctbp1 | GGTTTCCGAGGTTTCCTGGG | 1250 |
| Ctbp1 | GGAATTTGGGCAGCCTGAGA | 1251 |
| Ctbp2 | GTGAGAATAGAGGACCACGA | 1252 |
| Ctbp2 | GGGATGTGATGTGTTGGACA | 1253 |
| Ctbp2 | GGTCTTCAAAGTTGTGACCT | 1254 |
| Ctbp2 | GCACACAGGACAGACCTTGC | 1255 |
| Ctbp2 | GAGACACATTCATCTCCATG | 1256 |
| Ctbp2 | GTGTCACACTCCTCCCTAAA | 1257 |
| Ctbp2 | GAGTAGTGGGTTGGCCACCA | 1258 |
| Ctbp2 | GCGCCTCCCTTGAGACTCTG | 1259 |
| Ctbp2 | GAGCACAGCCACTGGAAAGG | 1260 |
| Ctbp2 | GTGTGCTTCTAAGCCCAGGC | 1261 |
| Ctcf | GAGTCACATTCCAAGGCTAT | 1262 |
| Ctcf | GTCGGAGAAGTGAGAGAGTG | 1263 |
| Ctcf | GGGATTAAGTACCACCGACT | 1264 |
| Ctcf | GTAACCTTAGGACTGCTTTC | 1265 |
| Ctcf | GGTATCAGAAGCCAGGAATA | 1266 |
| Ctcf | GCAAATAAAGGCATTGTCTT | 1267 |
| Ctcf | GATTAGAACACCTGCCAATA | 1268 |
| Ctcf | GGGACAGAGTCACCTCAGTC | 1269 |
| Ctcf | GGTGTGGTCTGCTATATCTC | 1270 |
| Ctcf | GGCATTGTCTTTGGAAAGAA | 1271 |
| Ctnnb1 | GTGAAGGAAGCGGGAGGTGA | 1272 |
| Ctnnb1 | GAGTAAACTCTGCTGCTGGC | 1273 |
| Ctnnb1 | GTTGATGACGTGTTTCTTTC | 1274 |
| Ctnnbl | GTCTTCCTTCCCAGGGTTAT | 1275 |
| Ctnnb1 | GGTCAGTAGAACCAGGCGTG | 1276 |
| Ctnnb1 | GGAGGTGATGGGTACGGAGG | 1277 |
| Ctnnb1 | GGATCCTATCCCAATAACCC | 1278 |
| Ctnnb1 | GCTAGAGGAATATGAATACA | 1279 |
| Ctnnb1 | GAAGCGGGAGGTGATGGGTA | 1280 |
| Ctnnbl | GGTAACACACTTCACATAGA | 1281 |
| Cux1 | GTGGCAGGGCTGCAAAGAAG | 1282 |
| Cux1 | GACGCAATGTACGTCATATA | 1283 |
| Cux1 | GGGTGGCAGGGCTGCAAAGA | 1284 |
| Cux1 | GGCCATCTACGTTTGTGCGG | 1285 |
| Cux1 | GTGCAATTGTGTCGTGGTAA | 1286 |
| Cux1 | GAGGGCTCATATGATTACAA | 1287 |
| Cux1 | GTCACCCTCCTTCCTGAGGG | 1288 |
| Cux1 | GAAGTCTATGCAGCAAACCA | 1289 |
| Cux1 | GTTGTCTTTGTGGGTGTCGA | 1290 |
| Cux1 | GAACTCGCGCGCGCTAAAGA | 1291 |
| Cux2 | GGTAAATATGCAGGCGACAA | 1292 |
| Cux2 | GGATGCTTGCTGCGTTTCTA | 1293 |
| Cux2 | GGACAATAGATCAATACCGT | 1294 |
| Cux2 | GCAGGAATTTATTGCACCAC | 1295 |
| Cux2 | GCCACTCGGAATTGCTAACT | 1296 |
| Cux2 | GTCTTTCTGAGGCCCTGGGA | 1297 |
| Cux2 | GCCTCTGTGGGACACACTGC | 1298 |
| Cux2 | GAGATAGCGTCTGCTCCATC | 1299 |
| Cux2 | GCGAATTTATGAGCCTTTAA | 1300 |
| Dbp | GAAAGAAGTGGGCTTCGGGA | 1301 |
| Dbp | GTGTTGGAGGGTCAGGTGAG | 1302 |
| Dbp | GGCATATCCCTTCATCTCAT | 1303 |
| Dbp | GGCGCAGTTCACTGAGTCGG | 1304 |
| Dbp | GGCGGGCGTAATCCTCGTTG | 1305 |
| Dbp | GTGAGGAAACTCAGAACAGG | 1306 |
| Dbp | GCTGAGAATGGCCAGGCCGT | 1307 |
| Dbp | GGTGTCAGTCACCTGGAGGG | 1308 |
| Dbp | GGCCTTCTTCCCTCCCTACA | 1309 |
| Dbx1 | GCGAAAGTGAGGGTTCGCGG | 1310 |
| Dbx1 | GTGTACGTGCAAGATCTGTT | 1311 |
| Dbx1 | GAGAAGTGTGCAGCCCTGCC | 1312 |
| Dbx1 | GAACGCACTAAATTTATCTG | 1313 |
| Dbx1 | GGACTCACTGTATAGCAGAG | 1314 |
| Dbx1 | GGAGGGTAGCTAGCCTTCCA | 1315 |
| Dbx1 | GTGGAATTCCCAGCCCGGTT | 1316 |
| Dbx1 | GGAAGAACTAAGTTCACACA | 1317 |
| Dbx1 | GACAGGTTTGCGCTAGCTAC | 1318 |
| Dbx1 | GTGGCAAAGAGCGAAAGTGA | 1319 |
| Dbx2 | GTTAACAGAAGGGAATAAAG | 1320 |
| Dbx2 | GATCAGACAATTCTGTGCTG | 1321 |
| Dbx2 | GGATGCTTCAAGACAAAGGA | 1322 |
| Dbx2 | GGAGATAGGTGCACTGTGTC | 1323 |
| Dbx2 | GAAAGGCAAAGTAAGGGTGG | 1324 |
| Dbx2 | GCGACCAAGTACATGTACCC | 1325 |
| Dbx2 | GATCTAGCTGAGAACCACAA | 1326 |
| Dbx2 | GGACTCCAGCAGCAGGGTCA | 1327 |
| Dbx2 | GTAACTATTGAGATGAGTGG | 1328 |
| Ddit3 | GGATTGGCCACCAGTGGCCT | 1329 |
| Ddit3 | GTTCAGGAAGGACAGCCGTT | 1330 |
| Ddit3 | GCACAGCAGTGGCCAGACAC | 1331 |
| Ddit3 | GTCAATCCAGGTGAACAAAT | 1332 |
| Ddit3 | GGAGTCAGGAATGTCAGGTC | 1333 |
| Ddit3 | GCAATTGCTTGGTGACCTGT | 1334 |
| Ddit3 | GCCGTGAGACTCCTGAGTGG | 1335 |
| Ddit3 | GAGAAGCGGGTGGACTATCA | 1336 |
| Ddit3 | GACATGTTGACCTGGAGAGG | 1337 |
| Ddit3 | GAACTCAGACAGCTAGAGGC | 1338 |
| Deaf1 | GACAAAGGTAGACTATATGT | 1339 |
| Deaf1 | GGTGTGATATGGTTGTATAC | 1340 |
| Deaf1 | GGCTTCTAGAGCTGAAGTGG | 1341 |
| Deaf1 | GTGTGCTCAGGATGAGCCAT | 1342 |
| Deaf1 | GCTGAGAGCACCTGAGAGTG | 1343 |
| Deaf1 | GATCACTGAGAGTCTAGGGT | 1344 |
| Deaf1 | GAGTGTATTGTGGATATGCC | 1345 |
| Deaf1 | GCATCTGAAGAGACCCAGGC | 1346 |
| Deaf1 | GCAGGTGAGCACTTCAGCCA | 1347 |
| Deaf1 | GTCTCCTCAGCAGCCAAGGA | 1348 |
| Dlx1 | GTAGACCCATGGTCGCTCTC | 1349 |
| Dlx1 | GTACAACAAATGGTCTAGTG | 1350 |
| Dlx1 | GTCGGATGGCCGGATTGCCT | 1351 |
| Dlx1 | GGGACAATTATTGCAGGTGA | 1352 |
| Dlx1 | GACGCCTAACCCTGAACCGC | 1353 |
| Dlx1 | GTTGAACCTACCTTCAGGGT | 1354 |
| Dlx1 | GAGGAGGAGGTGGGAAGCTG | 1355 |
| Dlx1 | GTGGTGTGTGGTAGTAGTGG | 1356 |
| Dlx1 | GCTTCCCACCTCCTCCTCCA | 1357 |
| Dlx1 | GCAATAATTGTCCCAGTGGT | 1358 |
| Dlx2 | GATTCTGAGGTTCCCTCCTT | 1359 |
| Dlx2 | GTAGGAGGTTGTTACAGGCC | 1360 |
| Dlx2 | GCCTTCAAAGTCGTTTGCAT | 1361 |
| Dlx2 | GTGGATCAAGCTACACTCTG | 1362 |
| Dlx2 | GCATCCACTTCCCAGGCTAC | 1363 |
| Dlx2 | GTCAGCCACTTTGCACCTGA | 1364 |
| Dlx2 | GGAGCCTTATGTCCTGTTGC | 1365 |
| Dlx2 | GAGATGTAAATCGTTAGACT | 1366 |
| Dlx2 | GCCTTCAGGACAGGCTTGAT | 1367 |
| Dlx2 | GGATGGACTCAGCGCAGTGA | 1368 |
| Dlx3 | GCTGGCTTTCTGTGTTCTTC | 1369 |
| Dlx3 | GTGTCTCTGTATGTAGTGTG | 1370 |
| Dlx3 | GCTGAGGCACAGTTGATGGA | 1371 |
| Dlx3 | GTTGATGGAAGGCCTGAAGC | 1372 |
| Dlx3 | GGCTGCAAGTCTTGCCTTCG | 1373 |
| Dlx3 | GGAGAAGCCTCCTTCCTCCA | 1374 |
| Dlx3 | GCTCCCAAACCTATCCTTGG | 1375 |
| Dlx3 | GTGGCTCTTCCATTCATGAA | 1376 |
| Dlx3 | GGGCTTAGGTGAGATGAGGA | 1377 |
| Dlx3 | GGTAAGCAGGCAGACAGGAA | 1378 |
| Dlx4 | GCTGGAGGGAATCTGCTGTC | 1379 |
| Dlx4 | GTAACGATGTTCAAGGTGCT | 1380 |
| Dlx4 | GATGTGCTTTGAGGCAGGGC | 1381 |
| Dlx4 | GTGATCCTGGAGCTCAGATT | 1382 |
| Dlx4 | GACAGGTCCAACTTTCTTTC | 1383 |
| Dlx4 | GCAACAGATGCTTGCATACA | 1384 |
| Dlx4 | GAACAGAGACAGGCAAATCC | 1385 |
| Dlx4 | GAATCTAGTTTGATGGCTCC | 1386 |
| Dlx4 | GGAGATCCTCTTTGTCTGGT | 1387 |
| Dlx4 | GATTCCCTCCAGCAGCCTCA | 1388 |
| Dlx5 | GTTTCCAGTATCAGGGTCAT | 1389 |
| Dlx5 | GCAAGGAACCAAGTCCGCTT | 1390 |
| Dlx5 | GGCAAGGAACCAAGTCCGCT | 1391 |
| Dlx5 | GGCCAGTCTTTCAGCACTTC | 1392 |
| Dlx5 | GCTCCCTGCTGAGACATGTA | 1393 |
| Dlx5 | GAGATTGGTGAATTTCAAAG | 1394 |
| Dlx5 | GGAGAACAGCATTGTCTTAG | 1395 |
| Dlx5 | GCAGCTCCAGATTCCAGAGA | 1396 |
| Dlx5 | GCAGGAGGTCAGTCCCTCTC | 1397 |
| Dlx5 | GAATCTTCTGGTTCCTCTTC | 1398 |
| Dlx6 | GACTGGGTGGGAGAAATCTG | 1399 |
| Dlx6 | GGTGTGTCTGGAGGTTGCGG | 1400 |
| Dlx6 | GGTAAGCTCTAGGAGCTTGC | 1401 |
| Dlx6 | GGTTCTCCTACCTGGTGGCT | 1402 |
| Dlx6 | GTCCATCTTTGAAACAGAAG | 1403 |
| Dlx6 | GCCTGTAATGATTATGGACT | 1404 |
| Dlx6 | GCTCCCTTGGGAGTAGAGTT | 1405 |
| Dlx6 | GAGTTACTGAACCGGCACCC | 1406 |
| Dlx6 | GTCGAATGGTTTGTCTCCAA | 1407 |
| Dmbx1 | GGAGCATGCATATGCAATTA | 1408 |
| Dmbx1 | GATGAGCATAGGACCCAACC | 1409 |
| Dmbxl | GACTGAACGGATGGAGGTCT | 1410 |
| Dmbx1 | GTGTGTGTTCTATGCTTGTG | 1411 |
| Dmbx1 | GCACACACCTCAGACACACA | 1412 |
| Dmbx1 | GGAAGAGGTCGTTATGCAGG | 1413 |
| Dmbx1 | GGGAAATGATGGACGCTGCC | 1414 |
| Dmbx1 | GTAGCCAATCTTGCACTACA | 1415 |
| Dmbx1 | GGGATCCTGGTGGGAGAGAA | 1416 |
| Dmbx1 | GGCTCCCTGCCTCTAACTCT | 1417 |
| Dmrt1 | GATAACAGATATTAGCTGCC | 1418 |
| Dmrt1 | GAACCTTCCGAGGATTGCGT | 1419 |
| Dmrt1 | GTACTGGTCCAAGCTGGAAG | 1420 |
| Dmrt1 | GCCTCTTGGCTAACAGAGAC | 1421 |
| Dmrt1 | GACACTGGCAGAGAGCAGGT | 1422 |
| Dmrt1 | GTGGTCCTGAGATGGAAGCC | 1423 |
| Dmrt1 | GAGGAGGCAGTGGTACACAT | 1424 |
| Dmrt1 | GAGCGCCAATGGTTGCTTGG | 1425 |
| Dmrt1 | GCAATTACATGTGTACCATC | 1426 |
| Dmrt1 | GGTAGGTGAATGGTTGCATG | 1427 |
| Dmrt2 | GTTCTCGAGAAGGTAACTAA | 1428 |
| Dmrt2 | GGTGGTGGATAATACTAGGA | 1429 |
| Dmrt2 | GTGTATGAACCAGTCAGATG | 1430 |
| Dmrt2 | GCAGAGAGTAGAGCCGGGAG | 1431 |
| Dmrt2 | GATAGGGAGCCCTAAGACAG | 1432 |
| Dmrt2 | GAACTTAAACGCACCCACCC | 1433 |
| Dmrt2 | GGCAAAGACCAGGCTCTCTA | 1434 |
| Dmrt2 | GATCATGTGGATAACGGGCT | 1435 |
| Dmrt2 | GACCACAAATGAGGAAACTA | 1436 |
| Dmrt2 | GTGGGAAAGTGGTTCCCTGG | 1437 |
| Dmrt3 | GAGGAGTTGATAGTTGTTCC | 1438 |
| Dmrt3 | GTTACAATAGACTTTGAGGC | 1439 |
| Dmrt3 | GATGTGCACTGGAGTGAAAC | 1440 |
| Dmrt3 | GGGTGAAAGTTAACGTAAAC | 1441 |
| Dmrt3 | GGGAATTGAGGGTACTCCGC | 1442 |
| Dmrt3 | GAATGGCTGAGGCCAAGGGT | 1443 |
| Dmrt3 | GCCAAGGGTGGGAAGGAAAG | 1444 |
| Dmrt3 | GCTTTAACAACTCAGTGGGA | 1445 |
| Dmrt3 | GAAGGGACCAGGGAAGGAAG | 1446 |
| Dmrt3 | GAAGGAGCCAACGGAAGTCC | 1447 |
| Dmrta1 | GTGCAGACTTCATCTAGGAA | 1448 |
| Dmrta1 | GCGGTTTCTTGCTCTGGGAC | 1449 |
| Dmrta1 | GCTCTCTGTTTCTACTAAGT | 1450 |
| Dmrta1 | GGGCGGAGAGTGGGACTTTC | 1451 |
| Dmrta1 | GTCTAGACTCAGAGGCTCAC | 1452 |
| Dmrta1 | GACAGGTTAATTCAGAGTCA | 1453 |
| Dmrta1 | GAGCACATGCAGATTATACA | 1454 |
| Dmrta1 | GAGGACCTAGGGCGGAGAGT | 1455 |
| Dmrta2 | GCTCCGAGGTAGTTGAGAGC | 1456 |
| Dmrta2 | GCAGAAGCTAACATCAGGAA | 1457 |
| Dmrta2 | GAGTGTGCATACTCGCGACC | 1458 |
| Dmrta2 | GACTGTGTCACCCTCCATGC | 1459 |
| Dmrta2 | GAAAGGCAAGGAGGGCACAG | 1460 |
| Dmrta2 | GGCATTCACGTGAAGAATTA | 1461 |
| Dmrta2 | GCTTGGACCCACGTTCCTCC | 1462 |
| Dmrta2 | GCATTAAAGGTGATAGAGGG | 1463 |
| Dmrta2 | GGAAAGGCAAGGAGGGCACA | 1464 |
| Dmrta2 | GGGAGCACATATCCAACAGG | 1465 |
| Dmrtb1 | GTCAGGGATGAAAGATTCGC | 1466 |
| Dmrtb1 | GCCTCCTGACTGGAGAGTCT | 1467 |
| Dmrtb1 | GCCCTGCTGTGAAATCTTTC | 1468 |
| Dmrtb1 | GGAATAAAGGCCATCCTGGA | 1469 |
| Dmrtb1 | GGGTGTCATCTGAAGTGGGT | 1470 |
| Dmrtb1 | GGTGTCATCTGAAGTGGGTA | 1471 |
| DMrtb1 | GCAAGTGAAGCAGGAATGAG | 1472 |
| Dmrtb1 | GACAAAGCATGTGTTCCAGT | 1473 |
| Dmrtb1 | GAAATCTTTCTGGTGATGCC | 1474 |
| Dmrtc2 | GTCTGTATCTACTCTCTCCC | 1475 |
| Dmrtc2 | GCAATCAGTGAGCTGGAAAG | 1476 |
| Dmrtc2 | GATGTCTCCTCATGTATTGG | 1477 |
| Dmrtc2 | GAGTGATGAGAGGTGTCCTT | 1478 |
| Dmrtc2 | GGTGCTATAAGGCCACACAT | 1479 |
| Dmrtc2 | GATTGTTGCCGCGGAGAAGC | 1480 |
| Dmrtc2 | GCAAGATAATTGCATTTCCC | 1481 |
| Dmrtc2 | GGATCAGCACCATGGCCAGG | 1482 |
| Dmrtc2 | GGTGCTTTCTGCCCAGCCTG | 1483 |
| Dmrtc2 | GAAGTGAACGCTTAAGCGGT | 1484 |
| Drd1a | GATCACCAGTCTGTGGAACT | 1485 |
| Drd1a | GCTCCAGCCTTGGCACACAG | 1486 |
| Drd1a | GGACTGACTGAGTCCATATC | 1487 |
| Drd1a | GGTGACCTGAGGGCAATTTG | 1488 |
| Drd1a | GTGGCAGCAAGACTGCCAGT | 1489 |
| Drd1a | GCCAGAATCTGGACGGTGAG | 1490 |
| Drd1a | GAGGCTGCTGAGTTTATGCC | 1491 |
| Drd1a | GGAGCACTTTCCCTCCCTGA | 1492 |
| Drd1a | GCAACAATGTAGTAACACTT | 1493 |
| Drd1a | GAATCTGGACGGTGAGAGGC | 1494 |
| E2f1 | GCAATCAGAAATGCTGATGG | 1495 |
| E2f1 | GATCAACACATTATCTGGGA | 1496 |
| E2f1 | GGGAGCCAGGAAATGAGTAA | 1497 |
| E2f1 | GTTAAGAATTGGAGAGGCCA | 1498 |
| E2f1 | GAGTAATGTGGTCAGAGTTG | 1499 |
| E2f1 | GAGCATTGGTTGCGGCGTGC | 1500 |
| E2f1 | GGCCGTCTCCAGTTCTCATG | 1501 |
| E2f1 | GCTACAGGGAGCTCTCAAGC | 1502 |
| E2f1 | GCTGCTTCTCAGGCCCTTTC | 1503 |
| E2f2 | GCGAATCTGTGAATGACCCG | 1504 |
| E2f2 | GATTCAGGAAGGAAGAGTGC | 1505 |
| E2f2 | GGTAAGACCAGGGAGTCGGA | 1506 |
| E2f2 | GACAGGCACAGCGTGGGTGA | 1507 |
| E2f2 | GTAAGACCAGGGAGTCGGAG | 1508 |
| E2f2 | GGAATGGAGGTGGCAGGGAG | 1509 |
| E2f2 | GGACCCTTCCATGGATTCCG | 1510 |
| E2f2 | GGAGTTTCGCTGCCTGGGAA | 1511 |
| E2f2 | GGAGTCACAGAGAAATCTCA | 1512 |
| E2f2 | GAGAAAGCTGCTACTCGGCC | 1513 |
| E2f3 | GGGATACGGTTTACGCGCCA | 1514 |
| E2f3 | GGTAAGCAGGACAIAAACCT | 1515 |
| E2f3 | GCTCTATGCAAATAGAGCCC | 1516 |
| E2f3 | GCTTTCCTGCGGACGTTGGG | 1517 |
| E2f3 | GGGCTAATCATGAAGCTGCC | 1518 |
| E2f3 | GTCTGGAGAGAGGAGGGTCC | 1519 |
| E2f3 | GGCAAAGTCCTACTCTCCCA | 1520 |
| E2f3 | GGTTTGCAAAGACTGGAATC | 1521 |
| E2f3 | GAGCAGGCTTCTTAGGAGGT | 1522 |
| E2f4 | GCTGAGGCTCTACCACATAG | 1523 |
| E2f4 | GTTAGACTGGGCTGGAGGGC | 1524 |
| E2f4 | GCGCCATTTCCTGTTGGGTG | 1525 |
| E2f4 | GGGCGTTACAGAGCAGGAAA | 1526 |
| E2f4 | GGTTCTCGCTTCTCAACTGC | 1527 |
| E2f4 | GGCTACAAGCAGGTGAGTGG | 1528 |
| E2f4 | GCACTAGGAAAGGGATTACA | 1529 |
| E2f4 | GTCAGTGGTGCAGTCCTACC | 1530 |
| E2f4 | GAGCCTCGTTGGCTGGGCTT | 1531 |
| E2f4 | GTCTCGGACCTCACAAACCC | 1532 |
| E2f5 | GGCAGGTAAGGAAAGAGCTG | 1533 |
| E2f5 | GCCTAGTAACGCACTCTCCG | 1534 |
| E2f5 | GTCTACTTCCTTCACCGTCA | 1535 |
| E2f5 | GCAGGTAAGGAAAGAGCTGG | 1536 |
| E2f5 | GTAACGCACTCTCCGCGGAG | 1537 |
| E2f5 | GAATGCCCAAATTAACAGTA | 1538 |
| E2f5 | GATCAGGTGCAAGTATTGTA | 1539 |
| E2f5 | GTAGAAGTAGAATACAACTG | 1540 |
| E2f5 | GGACTTAGTGAGGGCGGAAG | 1541 |
| E2f5 | GTCATACATCTTCATCAACC | 1542 |
| E2f6 | GTGTGTGGTGGGATGGGTTG | 1543 |
| E2f6 | GTTTGGCATTCAACAGAGGA | 1544 |
| E2f6 | GAGAGTTTCTCAGAGCAACT | 1545 |
| E2f6 | GACCTGGGACTTAGTGAGGG | 1546 |
| E2f6 | GCGCTGCGCATGTGCAAACG | 1547 |
| E2f6 | GAAGCTGCGGGAGTGAGACC | 1548 |
| E2f7 | GTGAACCCTGGTTAGCACCT | 1549 |
| E2f7 | GGACTTTGTTGCTTTAATTT | 1550 |
| E2f7 | GGAACAGTCAAGAATATCTC | 1551 |
| E2f7 | GTAATACACTCTGAAACCCA | 1552 |
| E2f7 | GTTTCTAGTAAGGACTAGCT | 1553 |
| E2f7 | GTGCTTTGTACTTACATAAG | 1554 |
| E2f7 | GCCAGGTGACACGTGAACCC | 1555 |
| E2f7 | GTCTTAGCCGTTCCGTGCAA | 1556 |
| E4f1 | GTGGAGTTGACCTGAGCAAG | 1557 |
| E4f1 | GGGCGTGGCTTGTGTTAAAT | 1558 |
| E4f1 | GTTGCAATGTCAGAATTTCC | 1559 |
| E4f1 | GCGAGCAGGGACTGAGCAAG | 1560 |
| E4f1 | GCCAGACATCAGGGCGGAAG | 1561 |
| E4f1 | GGAGTTGACCTGAGCAAGTG | 1562 |
| E4f1 | GGTCCAAAGTGAACTATCCG | 1563 |
| E4f1 | GCGGTCTAGCGCGTCAGTAG | 1564 |
| Ebf1 | GATGACGTTATGCAAAGAAG | 1565 |
| Ebf1 | GAAGAGCTGGACACCTGGGA | 1566 |
| Ebf1 | GAAGCCCTAGCTTAAGACTT | 1567 |
| Ebf1 | GGCTGCCAAGGACTCCTTGG | 1568 |
| Ebf1 | GCGGTCTACTAAAGTCGTAT | 1569 |
| Ebf1 | GTAGACAGATACACCGGAGG | 1570 |
| Ebf1 | GGGCAGAGGGAAGGAGATGG | 1571 |
| Ebf1 | GCCCAACAGCATTCGTGTCT | 1572 |
| Ebf1 | GGTCTGTCCAGGGAGGAAAG | 1573 |
| Ebf1 | GCTAAGGAGGAAATGAGTGG | 1574 |
| Ebf2 | GTTTGTCAAGGTCTTAGGGA | 1575 |
| Ebf2 | GTATGAGAGAAGCCGAGGAT | 1576 |
| Ebf2 | GAGCTGATCAAAGTCTCCTT | 1577 |
| Ebf2 | GATAACTGCCGAATGCAACT | 1578 |
| Ebf2 | GAAGCAATCATTTCGTGCGA | 1579 |
| Ebf2 | GCGGATTTGCCTCTAGATGC | 1580 |
| Ebf2 | GAACTTGTCACTGGGAAGGA | 1581 |
| Ebf2 | GACCCTACAGATTCATTCCC | 1582 |
| Ebf2 | GGTTATTCTCACGTAGCTGG | 1583 |
| Ebf2 | GCAGCTGATTGTCTGCTCCA | 1584 |
| Ebf3 | GACCTCTCCTAAAGGTCAGA | 1585 |
| Ebf3 | GCAGAGATGAAGTTGGGAAA | 1586 |
| Ebf3 | GGGTGGAGACCCTTCCTGGA | 1587 |
| Ebf3 | GGCTCCTCTGCAGCAGGCTA | 1588 |
| E3f3 | GCTCACACTGGGTGAGCGAC | 1589 |
| Ebf3 | GGCAAAGCCTGCTGAATACA | 1590 |
| Ebf3 | GAGGAGATTCCAGGAGAGGG | 1591 |
| Ebf3 | GGAATACTTCCCACCCTCCA | 1592 |
| Ebf3 | GGAGGAACCTGTCTCCGACG | 1593 |
| Ebf3 | GGGAGTGTGGATCCCTAGAA | 1594 |
| Egr1 | GAGAGATCCCGCTGGTCTCC | 1595 |
| Egr1 | GGTTGAGGATCCCACCTTTG | 1596 |
| Egr1 | GGAGACTGGGCAAAGTCAAG | 1597 |
| Egr1 | GAGAGCCTTAGACGCAGTGA | 1598 |
| Egr1 | GCAAAGAGCCCAGGAGGGAC | 1599 |
| Egr1 | GTGGGAAGGGTCTGTAGGTA | 1600 |
| Egr1 | GGGAGGGCTTCACGTCACTC | 1601 |
| Egr1 | GCCCTCCCATCCAAGAGTGG | 1602 |
| Egr1 | GGATCTGTTGGTTCTTGTGA | 1603 |
| Egr1 | GTCACTTTCCAGGTGTCACC | 1604 |
| Egr2 | GGGCGTTTGAAGTAATGGCG | 1605 |
| Egr2 | GAAGCTCTAAGCAAGGGCGT | 1606 |
| Egr2 | GGTGTGTAGTGTGTAGCGTA | 1607 |
| Egr2 | GATGAAGGCAGTGTCTTCCT | 1608 |
| Egr2 | GAAGTGGTTCCATACCATCA | 1609 |
| Egr2 | GTAGCGTAAGGTGTGTTGAG | 1610 |
| Egr2 | GCTCCGGGATCTACGTAGCC | 1611 |
| Egr2 | GCAAATAGAGGTCCCGGCGG | 1612 |
| Egr2 | GTAACCTGAGTCCCACCGCC | 1613 |
| Egr2 | GGCTCGGAGTATTTATGGGC | 1614 |
| Egr3 | GCTACGTCACGGAGCTTTCC | 1615 |
| Egr3 | GTTTGGAGGAGAACATTGGG | 1616 |
| Egr3 | GAGTGGGAGTGTTGACAAGA | 1617 |
| Egr3 | GTTGTCCTCATTGCTGCCTG | 1618 |
| Egr3 | GGCTCAGATAAATAGACTGG | 1619 |
| Egr3 | GGCTGGAGAGCCAGGCAATT | 1620 |
| Egr3 | GCAAAGAGGGTAATCCTCTC | 1621 |
| Egr3 | GGCACCCTCAGGCAGCAATG | 1622 |
| Egr3 | GTGTTGACAAGAAGGAAGAG | 1623 |
| Egr3 | GAATCACACCGGGTTGGCGG | 1624 |
| Elf1 | GAGTAACATAATTAGATGGC | 1625 |
| Elf1 | GTGGACCCAATTATTCTGCT | 1626 |
| Elf1 | GGCTCAAGGCTTTCAGCATA | 1627 |
| Elf1 | GCCATATATCCCTTCATATA | 1628 |
| Elf1 | GGTCAAACATGCAAATGCAC | 1629 |
| Elf1 | GACATCAGIGAGCGGGATCG | 1630 |
| Elf1 | GGATGGCTGACTGAGCACTG | 1631 |
| Elf1 | GCAAGAAGTCCACTGTTCAC | 1632 |
| Elf1 | GGGTTAATGAGTAGCCAGGT | 1633 |
| Elf1 | GCAGCTTGTTCCAAGGTGTA | 1634 |
| Elf2 | GATTAAGCTACATATCCTTG | 1635 |
| Elf2 | GGTGAAGGAGCGCGTGTGTG | 1636 |
| Elf2 | GAGGATCGTTTATTAGCCAT | 1637 |
| Elf2 | GACAGTAATATAACGCGATA | 1638 |
| Elf2 | GAGGTAAGGTTAGGATTACT | 1639 |
| Elf2 | GTCCCTGGAGGTCTTGGGAG | 1640 |
| Elf2 | GTTGGGCGCTGAGAAGAGGG | 1641 |
| Elf2 | GCTGCAAACGCAGGACATCC | 1642 |
| Elf2 | GGTCCCTGGAGGTCTTGGGA | 1643 |
| Elf2 | GGGAGTATAAATAGCCGGCC | 1644 |
| Elf3 | GCAGCCCTGACCTAGAGGAA | 1645 |
| Elf3 | GCAGATACTAATGGAGTGGG | 1646 |
| Elf3 | GCAGGCAGATACTAATGGAG | 1647 |
| Elf3 | GACGTACGCCGAAGACCTGG | 1648 |
| Elf3 | GCTTCAGCAACCATCGCGTT | 1649 |
| Elf3 | GAGTCATTACAAAGACAAAC | 1650 |
| Elf3 | GGACGGAATCAATACTCAGG | 1651 |
| Elf3 | GCTGGTTCTCCCACATTCCA | 1652 |
| Elf3 | GAGAGCGCCACAGGCACCAA | 1653 |
| Elf5 | GGAAAGCTTCACTATGCCTG | 1654 |
| Elf5 | GCAAATCTCTAGCCATGGGT | 1655 |
| Elf5 | GACGGCCTAGGCAGTCATCT | 1656 |
| Elf5 | GAGGCCTTACTCAGGCTGCC | 1657 |
| Elf5 | GAAAGCTTCACTATGCCTGT | 1658 |
| Elf5 | GGAAAGGCCTAGGCTGGGTA | 1659 |
| Elf5 | GTGTAGGCAGAGCAGAGGGC | 1660 |
| Elf5 | GGTGTAGCAGGGTCCTGGAA | 1661 |
| Elf5 | GGAACGGAACCCACGAAAGG | 1662 |
| Elf5 | GCCTGAGATTGAGAGAGGAA | 1663 |
| Elk1 | GTAGGACTCAACTCTGTGGA | 1664 |
| Elk1 | GTGCTTTAATATTGGAGGCT | 1665 |
| Elk1 | GAAACAGGACTTATTTAGAA | 1666 |
| Elk1 | GCCAAGGATCCTAAGCACAG | 1667 |
| Elk1 | GTGTACAGCACCACCTACTT | 1668 |
| Elk1 | GCGTCCTCCTGCTTGCTGAT | 1669 |
| Elk1 | GCAGTCCTCCTTGACCCAAT | 1670 |
| Eik1 | GCTGGGAAGATGCAGTCAAT | 1671 |
| Elk1 | GACAGGAGAAAGCCAAAGAA | 1672 |
| Elk1 | GGACAACGTATACTGAACCG | 1673 |
| Elk3 | GAGTTTAGGGACAGGAGGGA | 1674 |
| Elk3 | GATCCTGGCCATTGTCCTCA | 1675 |
| Elk3 | GTACCCTGTGGTTTCAAGAC | 1676 |
| Elk3 | GAAGAGGCTTAAGTTATTTG | 1677 |
| Elk3 | GTCGGATAGAGTTACTGTCG | 1678 |
| Elk3 | GAGAGTTGGGCATTGCTCGG | 1679 |
| Elk3 | GGCTGGAGGAACTGTATACA | 1680 |
| Elk3 | GCGTACTTATCCCAGACCAA | 1681 |
| Elk3 | GACACAAGGCTCCTAGTTTG | 1682 |
| Elk4 | GTTGTCATCTTCTCTTTAAC | 1683 |
| Elk4 | GATGGTACAAGGTAGACACT | 1684 |
| Elk4 | GAAACAAGTCACACTTGGTC | 1685 |
| Elk4 | GTGCACGGGACGGACTAACA | 1686 |
| Elk4 | GGACCAAGCTAAGTTGGTAA | 1687 |
| Elk4 | GAAATCAACACCCAATTCCA | 1688 |
| Elk4 | GGTAGACACTTGGTAAATAG | 1689 |
| Elk4 | GGACATTCGTACTTCCTCGC | 1690 |
| Elk4 | GGCTTAGTTATCTTATGCTA | 1691 |
| Emx1 | GGAGCCTGAGGATGACCTGT | 1692 |
| Emx1 | GCAGAGATCCGGAGAAGGCA | 1693 |
| Emx1 | GGTCTCCTTGGAGCAAGGTC | 1694 |
| Emx1 | GGAGTGGCATCCTAGCTTCT | 1695 |
| Emx1 | GTTCTCTGGAGAATCTAGGC | 1696 |
| Emx1 | GTCGCATATGGCGGGAGAGG | 1697 |
| Emx1 | GATGCAGAGTGGAGGGTAGG | 1698 |
| Emx1 | GAGAGCCCTAACACCGAGTT | 1699 |
| Emx1 | GCTTCTCCAGACCAAGGCTC | 1700 |
| Emx1 | GCAGAGTGGAGGGTAGGAGG | 1701 |
| Emx2 | GTCTCCTGTTTGGTTTCTTG | 1702 |
| Emx2 | GCTAATGATGCTAATGCTGG | 1703 |
| Emx2 | GGCCTCCAGTCTCTTGCATG | 1704 |
| Emx2 | GAAGCGGGTTAGCCCTTGCC | 1705 |
| Emx2 | GCTAGGCCATCTATGAGCTC | 1706 |
| Emx2 | GGTGTGGGTGCAGTAGGAGG | 1707 |
| Emx2 | GACATCTGTTGTCCCAGGGC | 1708 |
| Emx2 | GAAGACTGGAGCCCAAAGAA | 1709 |
| Emx2 | GACTGCAAACGCGTGGACCC | 1710 |
| Emx2 | GGAAAGGAGTCTTGGGTCCT | 1711 |
| En1 | GACTTTGCGGATAAATAATC | 1712 |
| En1 | GTTCTGCCAGGATCTCCAAC | 1713 |
| En1 | GTGGGTGAGAAGCTACAGCG | 1714 |
| En1 | GAGAATCTCCCGACTTCTCT | 1715 |
| En1 | GTGAGGGCAACTGGAGATTT | 1716 |
| En1 | GCCAGGATGGCAGACAGGTA | 1717 |
| En1 | GATCCGAGAAAGCTAGAATT | 1718 |
| En1 | GTCAGAAACTATGACATTTG | 1719 |
| En1 | GCTTGCCAGGACGTCAGCAC | 1720 |
| En1 | GTGGAGAAGCCTCAGAAAGT | 1721 |
| En2 | GTGCAGGAGACGCATGCATA | 1722 |
| En2 | GAGGCACGTGTCCAGGAGAC | 1723 |
| En2 | GAACTGCCAGGTCCTGGTGA | 1724 |
| En2 | GAGCCTACAGAACCCAGGCA | 1725 |
| En2 | GTGGCCTGGTGGCTCAACAT | 1726 |
| En2 | GCAAGGGCAATAACTCCCAA | 1727 |
| En2 | GGGCACGGCCACTTTAAAGG | 1728 |
| En2 | GTGGCTCAACATAGGAAATG | 1729 |
| En2 | GCCTCCTATAAGGAACTGCC | 1730 |
| En2 | GGCCTGGTATGTAAGTGGGA | 1731 |
| Eomes | GATGATACCATCTTGGCCTG | 1732 |
| Eomes | GTGTTTCTTTAAGCGTCTTT | 1733 |
| Eomes | GCTTGGAAACTTGTGAGCGG | 1734 |
| Eomes | GGTGTTTCTTTAAGCGTCTT | 1735 |
| Eomes | GACTGTTTGCGGAAACGCAG | 1736 |
| Eomes | GGCACCGTTCAGACCCACTC | 1737 |
| Eomes | GCCCGAGACCAAATCGGAGC | 1738 |
| Eomes | GAGGGTGTGCGCAGAGACTT | 1739 |
| Eomes | GCTCTATGGCGCCGGAGAAA | 1740 |
| Eomes | GTCCTGCTGTTTGTGCACCC | 1741 |
| Ep300 | GCTCCTAAGTCTAGTGTGTA | 1742 |
| Ep300 | GTTTGGGATCCTCAAATATA | 1743 |
| Ep300 | GGTACCTGGCTGGAGAGCAG | 1744 |
| Ep300 | GAGTGAGGAGGGTACCTGGC | 1745 |
| Ep300 | GTTCCAAAGATCAACCTGAG | 1746 |
| Ep300 | GAACCTGCCTGAAACTTCCA | 1747 |
| Ep300 | GCCGCTACCGCTATCCTGTA | 1748 |
| Ep300 | GCTACCGCTATCCTGTAAGG | 1749 |
| Ep300 | GAATCCTCCTTACAGGATAG | 1750 |
| Epas1 | GAAAGCACGGTCCCTCAAAT | 1751 |
| Epas1 | GACTTGCATAGAGCAGAGCC | 1752 |
| Epas1 | GGGAGCCCACGGTGATACTG | 1753 |
| Epas1 | GTTAGCGCAGGACTGAGTAA | 1754 |
| Epas1 | GAAATCAGTTGACACACCTG | 1755 |
| Epas1 | GAATAAAGATGGTACGGTTT | 1756 |
| Epas1 | GAGCGCAGCTCCAGAGAAAG | 1757 |
| Epas1 | GAGGATTGTACGGCCGCCTC | 1758 |
| Epas1 | GACAAGAACAAGAGCCGACA | 1759 |
| Epas1 | GGGCGATACCTGTAACCCGC | 1760 |
| Erf | GAGAGTGGGTAGGAGAAGTA | 1761 |
| Erf | GCTGAATAGGAACCCAACAA | 1762 |
| Erf | GCTGCAGCAGATAGGAGGAA | 1763 |
| Erf | GAGTTACCAAAGGAAGAGAT | 1764 |
| Erf | GACCAAAGGCCCGAGCGTAG | 1765 |
| Erf | GAGGAAAGGAATTATATGAA | 1766 |
| Erf | GGAAGTGACCAGAATGCATT | 1767 |
| Erf | GATGCGGCAAGCAAGAGGGA | 1768 |
| Erf | GCGCACTCACACACGCTTGC | 1769 |
| Esr1 | GTCACTGAGCATCTTATTCA | 1770 |
| Esr1 | GACAGTAGTCAGTAGGCTAT | 1771 |
| Esr1 | GTTTACAGACAGTAGTCAGT | 1772 |
| Esr1 | GAGCGTGCAAACTATGGGTT | 1773 |
| Esr1 | GCATCTGCTGTCTTGAGGTT | 1774 |
| Esr1 | GTGGAAGTAAGAATGGTATC | 1775 |
| Esr1 | GTTTGGTCCAGAGTCTGCAC | 1776 |
| Esr1 | GACTCTACTCTTAGAGAAGC | 1777 |
| Esr1 | GGAGAATGATGTTGGGTGTT | 1778 |
| Esr1 | GAGTGAAGTGTTGGGTCGGG | 1779 |
| Esr2 | GTGAGAGAGACAGGGAGACA | 1780 |
| Esr2 | GCTGGGTTAAGCTTGCACTG | 1781 |
| Esr2 | GGCAGGTAAAGGTGGTGTGA | 1782 |
| Esr2 | GTTCACAGAACCCAAGGAGG | 1783 |
| Esr2 | GAGTCCATCCTGGTGAGGAT | 1784 |
| Esr2 | GACCTGGAAAGAGTGTGGGA | 1785 |
| Esr2 | GCTCCCGGTTTGTGGTCACG | 1786 |
| Esr2 | GCTTTCATAGACATCTTCCA | 1787 |
| Esr2 | GGGACATTCTATCTCACAAA | 1788 |
| Esrra | GGGTGGAGTGCTCACTGATG | 1789 |
| Esrra | GCTCACTCTAACTAGTTATC | 1790 |
| Esrra | GGTGGAGTGCTCACTGATGA | 1791 |
| Esrra | GGAAGCCACATCGAACCTAC | 1792 |
| Esrra | GTTCTGGATCTCAGCCGGGT | 1793 |
| Esrra | GATGGGTGTGCCATAAGGGT | 1794 |
| Esrra | GTGCGATGTGAAGAATGGAG | 1795 |
| Esrra | GGGACACTGGTTTCAGCCCT | 1796 |
| Esrra | GTAGGCACAGGCCGACTCAA | 1797 |
| Esrra | GCGTCCTACTAGGAGGAACC | 1798 |
| Esrrb | GTCAATTCAGAAGTCAACCT | 1799 |
| Esrrb | GTATCTGTATCCCAGTAGAG | 1800 |
| Esrrb | GCAGGAACCACAAGGCTATG | 1801 |
| Esrrb | GTCATGTAGAAACCAACTCA | 1802 |
| Esrrb | GTCCACCTCTTACATCATGG | 1803 |
| Esrrb | GGTGAGTGAGTGACACCCTC | 1804 |
| Esrrb | GCTTCAGGTATTGGAATGAA | 1805 |
| Esrrb | GGTGGGACTGTTGGAAGGGA | 1806 |
| Esrrb | GCAGGGAGACTGTGTAGGTA | 1807 |
| Esrrb | GGTTAGTGGGCTCCAAGTGT | 1808 |
| Esrrg | GAGAGGGCCTGTGCTTCTGT | 1809 |
| Esrrg | GGGATATTAAGGCAGGATGC | 1810 |
| Esrrg | GAAGAGGGTTGAAGGTAAAC | 1811 |
| Esrrg | GACAAAGGTCTAAGGAGTAT | 1812 |
| Esrrg | GAGCGATTGTAAATGTGTGA | 1813 |
| Esrrg | GTGAATGCGTGCAATGAGCT | 1814 |
| Esrrg | GGTAAGACTTCAAATGCAGG | 1815 |
| Esrrg | GGGAGGGCGGGAAGTTGTTA | 1816 |
| Esrrg | GCCAACTCACGAGCCAGGAA | 1817 |
| Esrrg | GAAGACTTGCAGGAAGAGTG | 1818 |
| Esx1 | GTGGGACTACACTGTAGGGT | 1819 |
| Esx1 | GGAAACACTCCTATTTCTAA | 1820 |
| Esx1 | GACATTTGAATTGGCTTCTT | 1821 |
| Esx1 | GTAGTCCCACCCATTCCGAA | 1822 |
| Esx1 | GGCATAAAGGGTTTCTTGCA | 1823 |
| Esx1 | GAAGAAGCCACGGAAACCAA | 1824 |
| Esx1 | GGAGTGTTTCCATTCGGAAT | 1825 |
| Esx1 | GGGTGGGACTACATTGTAGG | 1826 |
| Esx1 | GTCTTGCCCAACCATTCCAC | 1827 |
| Esx1 | GTCTAGGCAGGAACCCTCGC | 1828 |
| Esx1 | GCCAGATAACAAGATGAGTG | 1829 |
| Esx1 | GAGCTGCCCTTTGTTTCTTA | 1830 |
| Esx1 | GTGAGGAAATCCCTGTATAA | 1831 |
| Esx1 | GACGGACTTTCCCGAGACTG | 1832 |
| Esx1 | GAGTGTCAATCTCTGGGTCT | 1833 |
| Esx1 | GCAAGAGTCACCTTTATACA | 1834 |
| Ets2 | GCCAGGCTAGGCTTTAACTC | 1835 |
| Ets2 | GGCACTTGGGTTGGGTGGTT | 1836 |
| Ets2 | GTTGGGTGGTTAGGCTTCTG | 1837 |
| Ets2 | GCTCAAAGGCTCTATCTTGG | 1838 |
| Ets2 | GGCTGAGAACTTGGTAGGGA | 1839 |
| Ets2 | GCCCTTTGAACCCAGAGGGT | 1840 |
| Ets2 | GTGGGCCAACCACAAAGCAG | 1841 |
| Ets2 | GTTCGGGCGTTATGCCCAGG | 1842 |
| Ets2 | GCAGGGCTGAGAACTTGGTA | 1843 |
| Ets2 | GGCAAGCTCAGGCAAGGCCA | 1844 |
| Etv1 | GGAGCCGAAAGGTGGAGTGG | 1845 |
| Etv1 | GGGTCAGCAATAAACAACAA | 1846 |
| Etv1 | GCAGGATTTATTGAGATACT | 1847 |
| Etv1 | GGACTTCTATCAACCTAGAG | 1848 |
| Etv1 | GGAGTGTTAGGACATGCTCT | 1849 |
| Etv1 | GAGAACGGGAGCCAAGAGAA | 1850 |
| Etv1 | GAGAGGTGGCGCTGGAAGAG | 1851 |
| Etv1 | GCCTTATCCGAATCACTCAA | 1852 |
| Etv1 | GAGTCAAATAGTTAACAGGT | 1853 |
| Etv1 | GAGCGAGAGATGCGAAGGGA | 1854 |
| Etv2 | GGACAAGATGGTGACATTTA | 1855 |
| Etv2 | GCATCAGCCTACGTCACAAT | 1856 |
| Etv2 | GTTGAGAAAGGAAAGTTCTA | 1857 |
| Etv2 | GGGTGACAGACAGCCAGATC | 1858 |
| Etv2 | GCCTGGAGGATGAATGAATT | 1859 |
| Etv2 | GGGTCAAGTTGCAGGGATGG | 1860 |
| Etv2 | GTTCGTGGCTCACCTCTGGC | 1861 |
| Etv2 | GTCTGAACTAGGAAGGACAG | 1862 |
| Etv2 | GCTCTGGGCTTATCTGCAAC | 1863 |
| Etv2 | GTGTCTGAACTAGGAAGGAC | 1864 |
| Etv4 | GGTCAGATTCTGGGTCTCCC | 1865 |
| Etv4 | GGAGGAACTCCGAGTCAGAC | 1866 |
| Etv4 | GTGACAAGCTGAGTTACCTC | 1867 |
| Etv4 | GAGGCGTGAGCTAACGCCAG | 1868 |
| Etv4 | GCCATCTTACTCCTTATGAT | 1869 |
| Etv4 | GGCTCAAACCGGCTTTCTCA | 1870 |
| Etv4 | GAACCCGTGGAGAAGCTGCC | 1871 |
| Etv4 | GGTCTCCATGAAGGTTCAGG | 1872 |
| Etv4 | GAAAGCTAAGAAAGACACCA | 1873 |
| Etv4 | GCCAGGGCTCTCCAGAGAAG | 1874 |
| Etv5 | GCAGGACGAGGAGTTGGAAG | 1875 |
| Etv5 | GTGTGAGTACGGGCTGCCCA | 1876 |
| Etv5 | GGTCAGCGAGTTTCTGTGTG | 1877 |
| Etv5 | GCAAGCAACACTGCTTCTCC | 1878 |
| Etv5 | GATAGCCACAGTATCATATG | 1879 |
| Etv5 | GGGATGAGAACAGGGAGGGA | 1880 |
| Etv5 | GACACAAGAAGAATGTCCCA | 1881 |
| Etv5 | GAGTCAGTGAAGCTCTTAAA | 1882 |
| Etv5 | GTGTTGCTTGCCAAAGGATC | 1883 |
| Etv6 | GAGTCTGGGAAACCCTCAGC | 1884 |
| Etv6 | GAACCAGGCTTGCTGGTCCT | 1885 |
| Etv6 | GGAGAAGGACATGTCAGGAA | 1886 |
| Etv6 | GAGAGATGAACCAGGCTTGC | 1887 |
| Etv6 | GGGAATACAGAGGTGAGTCT | 1888 |
| Etv6 | GTTCTTGGAGGGAACCTCCA | 1889 |
| Etv6 | GTCCAGTCACCTACGTCGGT | 1890 |
| Etv6 | GACCTGGGCCACGCACAGTA | 1891 |
| Etv6 | GGCATAGTGCATAGTGGCCC | 1892 |
| Etv6 | GGAAAGCCACCCTGTGGTAT | 1893 |
| Evx1 | GAGAGTGCTGGAGAAAGACA | 1894 |
| Evx1 | GGCAGGTGGGCCAGATTGAG | 1895 |
| Evx1 | GCGGCCAGTTCTTCGAGGAT | 1896 |
| Evx1 | GGTAGGGAGAGGTTCAAGTA | 1897 |
| Evx1 | GCATCGGCATAGGTAGGGAG | 1898 |
| Evx1 | GAACAGAATTGTGAGATCAA | 1899 |
| Evx1 | GCCCGGCTAGGAGGGATAGA | 1900 |
| Evx1 | GCAGCTGTGGGTAGATTGTG | 1901 |
| Evx1 | GAAGGTTATTTACTGAGCAG | 1902 |
| Evx1 | GACCCAGGAAGGAGACTAAA | 1903 |
| Evx2 | GAAATGCTATCCTCTGCTAA | 1904 |
| Evx2 | GGGCGCGTCAAGAATGTAAG | 1905 |
| Evx2 | GCTTGCCTGTAGAAATAAGT | 1906 |
| Evx2 | GGCCTGCCTTTAAATAAGAC | 1907 |
| Evx2 | GGTCTAGGCTAGGCTCCATG | 1908 |
| Evx2 | GTGTCTCAAGGCGGGAAGGA | 1909 |
| Evx2 | GCATCTGAGTCGGGCAGGGT | 1910 |
| Evx2 | GTAAGGGCCTAGGGTGGAGG | 1911 |
| Evx2 | GAAATCTTCCTAGGCCACTG | 1912 |
| Evx2 | GCTTTCTTGCTACGTGGCTG | 1913 |
| Ezh2 | GGTTCCTTTCGGCACCTTGG | 1914 |
| Ezh2 | GATAACTGAACAGGGAGTGG | 1915 |
| Ezh2 | GTTCGGCCCTCTGATTGGAC | 1916 |
| Ezh2 | GTATGAATACTAGCTTCTAA | 1917 |
| Ezh2 | GACACTGGTGGAAGTCATCC | 1918 |
| Ezh2 | GGCGACCAGATTTCTCTGAA | 1919 |
| Ezh2 | GAAAGCCATGGACAGGCAGG | 1920 |
| Ezh2 | GCAGCTCATTTCTAITCCTC | 1921 |
| Fev | GGATGATAGAGAAATTGTTG | 1922 |
| Fev | GCTCAGTCTGACAGGGATCT | 1923 |
| Fev | GAATCCTATGGAAACTGGGA | 1924 |
| Fev | GCATATATTGGCTGTGAGAC | 1925 |
| Fev | GCAGGGAGAAGAGTTCAGAG | 1926 |
| Fev | GATGGCTAGAAAGAGGGCTC | 1927 |
| Fev | GAGGGAGATGGCTAGAAAGA | 1928 |
| Fev | GCCAGACGAGACAGGAAACC | 1929 |
| Fev | GACTCTCA6CAAACATCGGT | 1930 |
| Fgf3 | GTTCAACGGCTACATCCTGT | 1931 |
| Fgf3 | GGATAGACCACTCCCACTTA | 1932 |
| Fgf3 | GCAGAGACAGAAATAAAGGT | 1933 |
| Fgf3 | GACTGCTTAAGATTTCTCAG | 1934 |
| Fgf3 | GGATTCATTTGTGACATCTT | 1935 |
| Fgf3 | GCATCCTTCATTTGAGTCCC | 1936 |
| Fgf3 | GTCACAGAGCCTTAGAGCCC | 1937 |
| Fgf3 | GGCAGAGACA6AAATAAAGG | 1938 |
| Fgf3 | GAAGGACCACACCAGGGTGC | 1939 |
| Fgf3 | GCCAGGCACAGGAAGGTAAC | 1940 |
| Figla | GGAAATATTTGCATGCATTC | 1941 |
| Figla | GGAACAAAGCCCGTAGACCA | 1942 |
| Figla | GGGCCAAATAAATAATGGAA | 1943 |
| Figla | GCTGCGACTTCTTACTTTCC | 1944 |
| Figla | GGCTGAGGGTGTGACTGCTG | 1945 |
| Figla | GTTGAGATCATTTCCTCACA | 1946 |
| Figla | GGACTCTAGGACAGGAAGAG | 1947 |
| Figla | GGCATCTGAAACCAGGAGGA | 1948 |
| Figla | GCACGTGTGCAGCCTGAACA | 1949 |
| Figla | GACTTAACCTGACTCACCTG | 1950 |
| Fli1 | GTAAACCGAGTCTCAATTGC | 1951 |
| Fli1 | GGAAAGAGGCCAGAGGCGTT | 1952 |
| Fli1 | GCTGCCATTCCTGAGCTGCA | 1953 |
| Fli1 | GGCGTTTGGCTTTGGATTTG | 1954 |
| Fli1 | GGTGGTAACCACATTAAACA | 1955 |
| Fli1 | GCTGGCCAGGAACAATGACG | 1956 |
| Fli1 | GAGGGTCTCCTTCCAGGCAC | 1957 |
| Fli1 | GAAGGGAAGAGCAAGAGGGC | 1958 |
| Fli1 | GCTTAACCCTTTCCTGCCTG | 1959 |
| Fli1 | GAGGGTGTGCACCACTGTGT | 1960 |
| Fos | GGATGGACTTCCTACGTCAC | 1961 |
| Fos | GATCTAAGGATGGAGTAGCA | 1962 |
| Fos | GCAGTTATGAGTGGAAGGCA | 1963 |
| Fos | GAGGGTTCAAGACAGGACTC | 1964 |
| Fos | GAGAGGATTAGGACAGCGGA | 1965 |
| Fos | GGCCGGTCCCTGTTGTTCTG | 1966 |
| Fos | GAAAGATGTATGCCAAGACG | 1967 |
| Fos | GATCCAAACCCAGCGGGAGC | 1968 |
| Fos | GTAAAGGAO6GAGGGATTGA | 1969 |
| Fosb | GGTGAGTCTTCAGGCTTTGA | 1970 |
| Fosb | GAATCCGTGACAAAGCTAGT | 1971 |
| Fosb | GTCACGGATTCTGTGTGACT | 1972 |
| Fosb | GTCTCCTGAGCTAAGTGGGA | 1973 |
| Fosb | GATATCTCCAGGTGTAGGGA | 1974 |
| Fosb | GAGTTGCACCTTCTCCAACC | 1975 |
| Fosb | GCGGGAAGGGAGAGTTTGGG | 1976 |
| Fosb | GTATAAGCAGACCTGGGATC | 1977 |
| Fosl1 | GTTCCCAATGAAGACAGCCC | 1978 |
| Fosl1 | GGAGTGTGTGTACGTGAcTT | 1979 |
| Fosl1 | GCTTTAATCCAGGCCTCTAC | 1980 |
| Fosl1 | GCTCTCTGCCTGTAGAGGCC | 1981 |
| Fos11 | GAGAGGAGCGGTCTTAAGTC | 1982 |
| Fosl1 | GAGCATCACCTCCTGCTCCC | 1983 |
| Fosl1 | GAGCGCCTACAGAAGGACAT | 1984 |
| Fosl1 | GCTTGTGATAGCTCCAGAGA | 1985 |
| Fosl1 | GCTGTCTTCATTGGGAACAA | 1986 |
| Fosl1 | GTCCTGAGACACAGTCAGTA | 1987 |
| Fosl2 | GGTAATCCCAAACAGTACTA | 1988 |
| Fosl2 | GTACAGATAAGCGCTGTACC | 1989 |
| Fosl2 | GGGCTGGAGAATAAAGAGTG | 1990 |
| Fosl2 | GGAAACGCAGGCGCTTTATA | 1991 |
| Fosl2 | GCGCCCTTGGTCTGTTCCAT | 1992 |
| Fosl2 | GCCTGAGTTTCCCGGCGACT | 1993 |
| Fosl2 | GGATACAGATGCACTGCATA | 1994 |
| Fosl2 | GTACACGCACGCACCAGCCT | 1995 |
| Fosl2 | GAGTCGCCGGGAAACTCAGG | 1996 |
| Foxa1 | GCACGGAGTGTGTGTGTGTT | 1997 |
| Foxa1 | GGAGTGACTTCTAGTCACAG | 1998 |
| Foxa1 | GTACGTTCCCGCAATGCCGG | 1999 |
| roxa1 | GCCCTGTCTTCTATGTCATA | 2000 |
| Foxa1 | GCCTTCACTTTCTGCTTAGT | 2001 |
| Foxa1 | GCTTAGTTGGTACCCAGATA | 2002 |
| Foxa1 | GGGTCAAGAATCAGGATGAG | 2003 |
| Foxa1 | GCAGGAACAAGGAAGCTTCT | 2004 |
| Foxa1 | GCAGATGCGTTCCAGCACCC | 2005 |
| Foxa1 | GATCAGTAGGAGAGCAGAGA | 2006 |
| Foxa2 | GAAGTAGTGCTGGCGGCAAT | 2007 |
| Foxa2 | GAAATAGTTGGCCCAAATCC | 2008 |
| Foxa2 | GTTTAGCTGCAGCCAATACC | 2009 |
| Foxa2 | GTGTGAGCTGATTATTCAAA | 2010 |
| Foxa2 | GGCTGGTCACTGAATGCCAG | 2011 |
| Foxa2 | GACCCATTTGAGTAGAAGGA | 2012 |
| Foxa2 | GAATTGCACAGCGTTAAGCA | 2013 |
| Foxa2 | GGAGCACTTGGGTGGAGATG | 2014 |
| Foxa2 | GATTATTCAAATGGGCTGCC | 2015 |
| Foxa3 | GTGTGGTCGAACTTGTTATT | 2016 |
| Foxa3 | GATCCACTCTTTAGGATAAC | 2017 |
| Foxa3 | GGTGGAGGAGGAGGAGGTGA | 2018 |
| Foxa3 | GCTCCCGCTCTGTTGCTCTA | 2019 |
| Foxa3 | GGAAGGAGGGAGGCAAACGG | 2020 |
| Foxa3 | GCTCCGTACAGAGTCAGGGT | 2021 |
| Foxa3 | GGAAACGTGCTGTTATCCTT | 2022 |
| Foxa3 | GAAGCCAAAGAAGGCAAGGA | 2023 |
| Foxa3 | GCGGATTTGAGGAAGGAGGG | 2024 |
| Foxa3 | GCTTCGCACACGGCCAGTCT | 2025 |
| Foxb1 | GATAGATATATTTGGACAGC | 2026 |
| Foxb1 | GTATTTAACCTAGTGGCATG | 2027 |
| Foxb1 | GTATCTTACTGGTTGCCATT | 2028 |
| Foxb1 | GAAAGAAACCAGCGCTGGCC | 2029 |
| Foxb1 | GAAGCATTGACCCGTCTCTG | 2030 |
| Foxb1 | GATTGGATGGGTTGTTCAAA | 2031 |
| Foxb1 | GCAATCGCGGCTTTAAGCCA | 2032 |
| Foxb1 | GGTCCAACTGATTTAATCTT | 2033 |
| Foxb1 | GAAGCTTGGTGGAGGTGGGA | 2034 |
| Foxb2 | GGTCTGCTGTTCCACAGCAA | 2035 |
| Foxb2 | GGTGTATCCTCTTGTTTCTT | 2036 |
| Foxb2 | GGATGACTAGACTTGAGCTC | 2037 |
| Foxb2 | GACCAGTGGAAATGGAGAAG | 2038 |
| Foxb2 | GCCTCTCAGCTGTAAGGTTT | 2039 |
| Foxb2 | GGGAAGTGAAAGCGAAGGGT | 2040 |
| Foxb2 | GGCTGCAGCTGCAGCTCAGA | 2041 |
| Foxb2 | GGAGCCAGAAGGTTCCCTGC | 2042 |
| Foxb2 | GCCAAACAGCAGGAGCCAGA | 2043 |
| Foxb2 | GGGCGTCCTAGGAATTCCTC | 2044 |
| Foxc1 | GACGGCAAAGTGATTGCCCG | 2045 |
| Foxc1 | GAGTCTGGGAGTGAGTGGGT | 2046 |
| Foxc1 | GGAAAGGAGAGGACAAGGGA | 2047 |
| Foxc1 | GTGGTGACACCACAGGAATG | 2048 |
| Foxc1 | GAGGGATATTCGGAAAGGAG | 2049 |
| Foxc1 | GCGGTATTGGAGGATCTGAG | 2050 |
| Foxc1 | GAACGTGAAGATGCAGTCTT | 2051 |
| Foxc1 | GTGTGTTAGTGAGGGAAAGA | 2052 |
| Foxc1 | GTTTCCACATTCCAGCGGGC | 2053 |
| Foxc2 | GAGTCGCTGTGCGTCAAGGT | 2054 |
| Foxc2 | GAGGGAAGGAGCACGCTTGA | 2055 |
| Foxc2 | GAGTCCTCCAAACAATTTCG | 2056 |
| Foxc2 | GAAAGCGCTGTCTGGAGGTT | 2057 |
| Foxc2 | GGTTTAAATTTGGCATACGC | 2058 |
| Foxc2 | GGCTGGGAGGGAAGGCTTAG | 2059 |
| Foxc2 | GTCCGGTGTAGATCTGGGTA | 2060 |
| Foxc2 | GAGATCTGGCTAAGAGCATC | 2061 |
| Foxc2 | GCTGGGAGGGAAGGCTTAGT | 2062 |
| Foxc2 | GTGGGAATGCCAAACTGGGA | 2063 |
| Foxd1 | GATCTAGTAGTCTCCTCTGA | 2064 |
| Foxd1 | GAGAAAGTTCACCCGCAGGA | 2065 |
| Foxd1 | GATCAATGAAGGTACAGAAC | 2066 |
| Foxd1 | GATTCTGAGAGCTAGGGACC | 2067 |
| Foxd1 | GGAGAAGAAGCCTGTTGTGC | 2068 |
| Foxd1 | GCTTTCAGGCCAAGGAGTGG | 2069 |
| Foxd1 | GGTAGGGTGTCCCAGCTCTC | 2070 |
| Foxd1 | GCAGACAGGCGTCCTAACCA | 2071 |
| Foxd1 | GGGCCTGTGACAAAGATGAA | 2072 |
| Foxd1 | GAAACAGCCCTTTAACCCTT | 2073 |
| Foxd2 | GGAGTGCACAGCAGGTATTG | 2074 |
| Foxd2 | GGGATGAGGTTAAGTTTCTT | 2075 |
| Foxd2 | GTGGCCAAGGCTCCAGCATA | 2076 |
| Foxd2 | GCAACACTGGCCCAGGGATG | 2077 |
| Foxd2 | GCGGTGCACACCAGGAAAGT | 2078 |
| Foxd2 | GCCAGAACATTCCACTTCCA | 2079 |
| Foxd2 | GCCCTATTCTCTGGGAGGGA | 2080 |
| Foxd2 | GGGTGCCCTATTCTCTGGGA | 2081 |
| Foxd2 | GCCTAAGGCGGAAGAACTGT | 2082 |
| Foxd2 | GCCACTGTGAGGCGCTGTTG | 2083 |
| Foxd3 | GACTTTGTCCGCCTCGTTGA | 2084 |
| Foxd3 | GCTGGAAACGGAGCAGGCAT | 2085 |
| Foxd3 | GTTATGACGTCTTTGTTTAT | 2086 |
| Foxd3 | GGGAAATCCCAGAGATGCTG | 2087 |
| Foxd3 | GGGCTCCAAGCAGCTCTGGA | 2088 |
| Foxd3 | GTTCAGGGAATTGTCAACAA | 2089 |
| Foxd3 | GCGGTCTTGGGTAAGTGGAG | 2090 |
| Foxd3 | GGCTTAATATCGATTTCTAG | 2091 |
| Foxd3 | GCTGACTAGACAGTCTTCTC | 2092 |
| Foxd4 | GCTGGCCTCTGACCTCTACA | 2093 |
| Foxd4 | GAACTTCCACAGTTTATGCT | 2094 |
| Foxd4 | GTAGTTGGTAAAGACAACGA | 2095 |
| Foxd4 | GGAACCGAGTCTCTCCAGCA | 2096 |
| Foxd4 | GAGGGAAGGAGCCATTTCTC | 2097 |
| Foxd4 | GCAGTGTGGATGCCTTACCA | 2098 |
| Foxd4 | GAACCGAGTCTCTCCAGCAG | 2099 |
| Foge1 | GCCAGTACCTTTCCTGAGCA | 2100 |
| Foxe1 | GGGTAAGAACTGGACTAAAG | 2101 |
| Foxe1 | GTCTACAGCTGAAGACGACG | 2102 |
| Foge1 | GGTGGAAGGTACAACCCAAG | 2103 |
| Foxe1 | GAATTCTGCTTCCCTCTGCT | 2104 |
| Foge1 | GAAAGCCTCCTCGCCGCATC | 2105 |
| Foxe1 | GAAGCAGAATTCTGGAAGAA | 2106 |
| Foxe1 | GCCGCATCAGGGTCCTTAGG | 2107 |
| Foxe1 | GTTTGCTGGCGCCTTTAAGG | 2108 |
| Foxe1 | GGAAAGAGACACACTGTGGA | 2109 |
| Foxe3 | GCTAGCAAAGACTGCTGGAG | 2110 |
| Foxe3 | GAGCGGGAACTAGAAGCATG | 2111 |
| Doxe3 | GCATCCTATGTAGCTGGTCA | 2112 |
| Foxe3 | GGGATGGTACTTACTGAGAC | 2113 |
| Foxe3 | GGGCTGGGAAAGCAAATTAG | 2114 |
| Foxe3 | GGGAAAGCAAATTAGAGGGC | 2115 |
| Foxe3 | GAGGAAAGCGAGAAAGGCTA | 2116 |
| Foxe3 | GGACAGTTACACACAGGGAA | 2117 |
| Foxe3 | GACTGACTCAGGGATGAGGG | 2118 |
| Foxe3 | GACCCAGAGGGACTAGACCA | 2119 |
| Foxf1 | GCGGATTTCAGAGTTAAGCG | 2120 |
| Foxf1 | GCAAGCCTGCGCGTCTAAGT | 2121 |
| Foxf1 | GGACAGACTTTAGAACTCTG | 2122 |
| Foxf1 | GAGCCCACTGAATAGCTACG | 2123 |
| Foxf1 | GGTGATTAGAGGATTCGCTT | 2124 |
| Foxf1 | GAGCCACAGGATCACAAGAA | 2125 |
| roxf1 | GGGTGTGGGAATGTGTGGCC | 2126 |
| Foxf1 | GGCACATCTGTGCGAGGGTC | 2127 |
| Foxf1 | GTCTGTCCTGGAGAAAGGAA | 2128 |
| Foxf1 | GACCCTCGCACAGATGTGCC | 2129 |
| Foxf2 | GGCACGGATCGCTAGGTTGG | 2130 |
| roxf2 | GGGCACGGATCGCTAGGTTG | 2131 |
| Foxf2 | GGGTCTGAGGAACAGAGGAA | 2132 |
| Foxf2 | GGGATCAGCATGAAATAAAT | 2133 |
| Foxf2 | GGAGCTTTGTGGGCAGACTT | 2134 |
| Foxf2 | GGAGTAATCGAGTCTGGCCA | 2135 |
| Foxf2 | GCTCAGAGAGAGATGGCCCT | 2136 |
| Foxf2 | GCCTGGGAAGATGGGAGACA | 2137 |
| Foxf2 | GTTGACAGATGGTGTCAGTT | 2138 |
| Foxg1 | GAATGCGAGTCTCTCAAAGC | 2139 |
| Foxg1 | GCTTTATGTGCAGAGGGAAG | 2140 |
| Foxg1 | GCCCAGCATTTCCCAGGGAT | 2141 |
| Foxg1 | GATTACCTTCAGAAGACAGA | 2142 |
| Foxg1 | GTGAAATGATTCGGTGTAAC | 2143 |
| Foxg1 | GTAGGAAGAGATCCAAGCAG | 2144 |
| Foxg1 | GCGCGCCACGTTGTAAGCAG | 2145 |
| Foxg1 | GCTAGAGAGATCTGTGAGCC | 2146 |
| Foxg1 | GGTGCCAAAGGTTGATTTCT | 2147 |
| Foxg1 | GCTCACAGATCTCTCTAGCT | 2148 |
| Foxh1 | GTGAGGGTCGGGTCTATCTG | 2149 |
| Foxh1 | GACTTTCCTGTGCTTCATGT | 2150 |
| Foxh1 | GCTTTAACTGGAACCAAGGA | 2151 |
| Foxhl | GTCATCCACTGTAGATTGAC | 2152 |
| Foxh1 | GTCATGGTGATGGGACTTTC | 2153 |
| Foxh1 | GAGGTTCCAGGIGAGAAGGC | 2154 |
| Foxh1 | GGTACAGTCATGAGTGGAGG | 2155 |
| Foxh1 | GCAGCAGTTTGGGTGATGGT | 2156 |
| Foxh1 | GGAGTCTGCTCAGGACTTGA | 2157 |
| Foxh1 | GATGGATTTGCCCGACCAAC | 2158 |
| Foxi1 | GCTTACTTACTTTGAACCTC | 2159 |
| Foxi1 | GGCAAATGAAAGCAATTCTG | 2160 |
| Foxi1 | GAGCATGTGTCAGTGCCTGG | 2161 |
| Foxi1 | GGATAAGCCACCTTTAAGCT | 2162 |
| Foxi1 | GTGACCTGGCACAACTGTCC | 2163 |
| Foxi1 | GAAGATGATGGCACCAAGAG | 2164 |
| Foxi1 | GTAAAGCAGAGAGAGAGGTT | 2165 |
| Foxi1 | GCCTAGCTCCCTCAGTGCCA | 2166 |
| Foxh1 | GAAATCGTCCTTGCTGAGGG | 2167 |
| Foxh1 | GACTCAGGAGAAGAAAGTAG | 2168 |
| Foxi2 | GTTAACGAGGCAGTATGACA | 2169 |
| Foxi2 | GTGCCTAAGGCAAGGGCATC | 2170 |
| Foxi2 | GAGTTCCAAGACTGTTTGTC | 2171 |
| Foxi2 | GACCTGTTTGTCATGTAGCC | 2172 |
| Foxi2 | GGTTACAGCTGTGGCACAGA | 2173 |
| Foxi2 | GCCCAGGCTACATGACAAAC | 2174 |
| Foxi2 | GGTTGGTTAAGTAAAGGCAG | 2175 |
| Foxi2 | GTCCAATCTGGCCTGGCTTC | 2176 |
| Foxi2 | GAGAGAGAGGCTGGTGGCTT | 2177 |
| Foxi2 | GAGAGCAGAGCCTTAGGAGC | 2178 |
| Foxi2 | GGCCACTTCCCTGCAGTGCT | 2179 |
| Foxj1 | GGGAAGCAGGGTGTTCAGAA | 2180 |
| Foxj1 | GAATGAGCAAGGCAGAGCAA | 2181 |
| Foxj1 | GAGACTTGGTCTGCAGAATC | 2182 |
| Foxj1 | GGGCAAAGACTTCAAGGGCA | 2183 |
| FoKj1 | GGCAGATGCAGAAGCAGGTA | 2184 |
| Foxj1 | GGCAACTCTCTGGAACTCTC | 2185 |
| Foxj1 | GGGAGGAATGCACTAGGGTA | 2186 |
| Foxj1 | GCTTCCAACCAATAGTTCGG | 2187 |
| Foxj1 | GACTTGGTCTGCAGAATCCG | 2188 |
| Foxj2 | GCTGTCTGGAAGAGAAAGAG | 2189 |
| Foxj2 | GTCAGTGAAAGATTGGATCG | 2190 |
| Foxj2 | GTAGTGAGACCTGAAGGACC | 2191 |
| Foxj2 | GATGCCAGCGTCCACGCTAA | 2192 |
| Foxj2 | GGAAGGGTAGTGAGACCTGA | 2193 |
| Foxj2 | GTCACTTGACTTTAGCACAA | 2194 |
| Foxj2 | GGTCTCACTCCGGGTCCTTC | 2195 |
| Foxj2 | GGGAGGGAAGAGGCTTTGTT | 2196 |
| Foxj2 | GAGAAAGGGAGCCATGCCTG | 2197 |
| Foxj2 | GGTCCTGGCTTGTGCGTATC | 2198 |
| Foxk1 | GACACAGACCTTCCAGTGCT | 2199 |
| Foxk1 | GATGAATCCAAGCACCCTTC | 2200 |
| Foxk1 | GGTGGAGCAATTGAGCACAC | 2201 |
| Foxk1 | GGGAATTAGCATCCCAGTGC | 2202 |
| Foxk1 | GAGGCTGGAGTTTAAAGTCC | 2203 |
| Foxk1 | GCACTGGAAGGTCTGTGTCT | 2204 |
| Foxk1 | GCTGACGGCCAAGTGIGGGA | 2205 |
| Foxk1 | GAGGGTGAGCTGGCACAGGT | 2206 |
| Foxl1 | GAGGCAGGATGTGGAGGGAC | 2207 |
| Foxl1 | GAAACACACTCCGACCCTCT | 2208 |
| Foxl1 | GAACAGAGACTGCCTCCTCA | 2209 |
| Foxl1 | GTTGAAGTCACAGAGGAAAG | 2210 |
| Foxl1 | GAATCTACAGCGGAATGTGG | 2211 |
| Foxkl1 | GGTTGGACACTTAAGGAATC | 2212 |
| Foxkl1 | GCACCGCCCACTTTAGTCGT | 2213 |
| Foxkl1 | GTGGGTGGGTGTGTGTGTGG | 2214 |
| Foxl2 | GCAGAGCCTCTAACTTCTGC | 2215 |
| Foxl2 | GACTTCTTGCTGTCCTTTGC | 2216 |
| Foxl2 | GATGAGACCCAGGGTCAGCT | 2217 |
| Foxl2 | GGTGGAGTGGCCGAACTTTG | 2218 |
| Foxl2 | GAGAGGTGATCCAAGCCTCT | 2219 |
| Foxl2 | GCATCTTCTCCTTCCAGCAT | 2220 |
| Foxl2 | GCTTCCCACTTTGAGATGAA | 2221 |
| Foxl2 | GCACAAGTGTCACGCGTGGA | 2222 |
| Foxl2 | GAAGTCAGCCTCTGGCCATC | 2223 |
| Foxl2 | GAAGGAGAAGATGCAGGTAA | 2224 |
| Foxm1 | GTTGAGTGTGGAGAATAATG | 2225 |
| Foxm1 | GCCTTCTAGTACACAATGGC | 2226 |
| Foxm1 | GCCACGTAACCGCAAGTCTA | 2227 |
| Foxm1 | GTATGAAGAGAGCTGCAGGG | 2228 |
| Foxm1 | GCAAGICACTTGCAATGACT | 2229 |
| Foxm1 | GCCAAGGCGTTGTCACAGAA | 2230 |
| Foxm1 | GGGAAGAAGGTCTGAGCCTC | 2231 |
| Foxm1 | GAAGAGAGCTGCAGGGTGGA | 2232 |
| Foxm1 | GCAAGCTTTGACCCTGAGGA | 2233 |
| Foxn1 | GACATGGGAGGGAAGTCACA | 2234 |
| Foxn1 | GCAGGCATGCCCACAGACAT | 2235 |
| Foxn1 | GTTAACCATCGTGTACAGAT | 2236 |
| Foxn1 | GGGTTCTGGGAAGCAGCACA | 2237 |
| Foxn1 | GAGGGAAGTCACATGGATTT | 2238 |
| Foxn1 | GTGCATGTCCCAACAGGCCT | 2239 |
| Foxn1 | GCTACACACTGCCACACATA | 2240 |
| Foxn1 | GGAGACACAAGCCTGAGTAC | 2241 |
| Foxn1 | GAGTCAATTCACCGTTCTCT | 2242 |
| Foxnl | GGACATGCTGTGTATGGATG | 2243 |
| Foxn2 | GGAGATTCTTGTATACCAAG | 2244 |
| Foxn2 | GTATTGCCTACACTGTATTG | 2245 |
| Foxn2 | GGTCTGGGTGTGGTCAGTCA | 2246 |
| Foxn2 | GCTTCATTTGGTTCCTGATG | 2247 |
| Foxn2 | GCTTTGTTGAGCAAGCAGAC | 2248 |
| Foxn2 | GGTGTGGTCAGTCACGGCAG | 2249 |
| Foxn2 | GCTCCACTTAGTTCAAAGTC | 2250 |
| Foxn2 | GGCAACAGTGATGCTATGTA | 2251 |
| Foxn2 | GACAAAGGTGCCCAGGCTTG | 2252 |
| Foxn2 | GCCCGGAAGGACCTATGGGA | 2253 |
| Foxn4 | GCCACGGTCGCATGTTGAAG | 2254 |
| Foxn4 | GCAGTGCATGACCCAGCCAG | 2255 |
| Foxn4 | GACCGGTTTACGTATTACTC | 2256 |
| roxn4 | GAGTTGGACAGCTCCAGAGA | 2257 |
| Foxn4 | GTGCAGTGCATGACCCAGCC | 2258 |
| Foxn4 | GAAGCAACGGGCTCTTTCTG | 2259 |
| Foxc8 | GCGAGCTCGGGAGACGAAAG | 2260 |
| Foxn4 | GATCAATCCTGTTAGGGAAC | 2261 |
| Foxn4 | GCCTGAACTTAAGGGTTCCC | 2262 |
| Foxo1 | GGTTCAGGATGAGTGGAGGC | 2263 |
| Foxo1 | GAAGACTTCACTCATCTTGG | 2264 |
| Foxo1 | GAGGCGGCAGTAGGTTGGTG | 2265 |
| Foxo1 | GCACCTTAAACGGTTCATAG | 2266 |
| Foxo1 | GGTGAAGACCCGTCGCTCTG | 2267 |
| Foxo1 | GTCCTCGGCACCTCTGGTTC | 2268 |
| Foxo1 | GCAGGTGTGCACAGGTAGGG | 2269 |
| Foxo1 | GACGTCACTGAGCATCTTAC | 2270 |
| Foxo1 | GCGAAGGCCAAATTCACAGC | 2271 |
| Foxo3 | GTCTGGAGCCCAGAGACTGG | 2272 |
| Foxo3 | GGGAGGAGGGAAAGGAGGTA | 2273 |
| Foxo3 | GTGCACACACCTGGACCACA | 2274 |
| Foxo3 | GCGTCGAACTAGCTTGGTGC | 2275 |
| Foxo3 | GGCAATATAGATGGTGATGT | 2276 |
| Foxo3 | GCATTCTGACCCTGAAGGTA | 2277 |
| Foxo3 | GAAGAGGAGCGAGAGGCGTC | 2278 |
| Foxo3 | GAGGCACGGATCGTGGGATA | 2279 |
| Foxo3 | GACAGCGGGAGGACTAGAGG | 2280 |
| Foxo4 | GAAGTAGCAAGTTACAGAAG | 2281 |
| Foxo4 | GGGATTCAGTTCTGGAGTTG | 2282 |
| Foxo4 | GTTTCCTCTGTCAGCTATGC | 2283 |
| Foxo4 | GTAGTCTTCGAGAACGACCA | 2284 |
| Foxo4 | GGTGGAACTTTAATGATTAG | 2285 |
| Foxo4 | GGTAAACAGAGACGTCTGGC | 2286 |
| Foxo4 | GGTCACTCTTGAGAGGGTCA | 2287 |
| Foxo4 | GTCTCTGTTTACCACTCGCT | 2288 |
| Foxo4 | GAAGGCCCAGTGTATGAAGA | 2289 |
| Foxp1 | GGGAAGGAATCACACCACCA | 2290 |
| Foxp1 | GCAGTGAGGGTTTCTAACCG | 2291 |
| Foxp1 | GGTGGCCTCTGGATCCGCAA | 2292 |
| Foxp1 | GACAGTCTTCTGAAGCAGGC | 2293 |
| Foxp1 | GTAATTTGTCTGTAGAACCC | 2294 |
| Foxp1 | GCAATTAAGAATTCACTCCA | 2295 |
| Foxp1 | GAAGGCTAGGAATCTTCTTC | 2296 |
| Foxp1 | GCTTTGGTGTTGATGACAGT | 2297 |
| Foxp1 | GTAGAGTAGTAGGGTCTCAG | 2298 |
| Foxp2 | GAGCTGCTGGCAAATGAAAC | 2299 |
| Foxp2 | GAAACTCTAACTGCTTGCTT | 2300 |
| Foxp2 | GTAGAGAAGAATGACTACAG | 2301 |
| Foxp2 | GACCACATACCTTGCCACGG | 2302 |
| Foxp2 | GGGTCTTGTGACTTGAATCT | 2303 |
| Foxp2 | GAGGACCCTGTCAGAATGAA | 2304 |
| Foxp2 | GTAGCCTAGCAGGGTTGGTG | 2305 |
| Foxp2 | GGCGCACACACAGGAGAGAA | 2306 |
| Foxp2 | GACCCTGTCAGAATGAAAGG | 2307 |
| Foxp2 | GAGAGAGGCGACTTGAGCAG | 2308 |
| Foxp3 | GGGTCTGTGGAAGCTGAGAC | 2309 |
| Foxp3 | GAGCAGGGACCATTAACTTT | 2310 |
| Foxp3 | GCTAAGGAAATACTGAGGTT | 2311 |
| Foxp3 | GAGAAGACAGACCCATGCTG | 2312 |
| Foxp3 | GGGATGAGGTCCTCTTACTT | 2313 |
| Foxp3 | GGCAGAGAGGTATTGAGGGT | 2314 |
| Foxp3 | GCCATTGACGTCATGGCGGC | 2315 |
| Foxp3 | GGCAACAAGGAGGAAGAGAA | 2316 |
| Foxp3 | GTAGCCTTTCTTTCCACAGA | 2317 |
| Foxp3 | GCCCAAGTGTACAGGGAGCA | 2318 |
| Foxp4 | GGAGGACTAAATTGGGTAGC | 2319 |
| Foxp4 | GGATGAACTGGGTAAGGACT | 2320 |
| Foxp4 | GAGCTTGTGTTTAGCACTTC | 2321 |
| Foxp4 | GGACCACGTGGACCAAACTT | 2322 |
| Foxp4 | GTAGCAATGAGAGACTGACT | 2323 |
| Foxp4 | GTTCCTTCCTTGCTCCCACA | 2324 |
| Foxp4 | GAGTTAATAAAGCCTCCCAT | 2325 |
| Foxp4 | GCCTCAATTAGGACAAGATG | 2326 |
| Foxp4 | GACTGAGTAGGCCTGAGTAG | 2327 |
| Foxp4 | GTGGGCCAGGAGCTGAAAGG | 2328 |
| Foxq1 | GAGAATTCATTCACCTTCTA | 2329 |
| Foxq1 | GGCCATAGAGAGGAAGTAAG | 2330 |
| Foxq1 | GGCCGAGGGACTGGTTGCAT | 2331 |
| Foxq1 | GACAAAGCATTGATTTGGCC | 2332 |
| Foxq1 | GATGGATTGATAAGTGCCTG | 2333 |
| Foxq1 | GCCTAACCAAGATCAAGGTA | 2334 |
| Foxq1 | GCACGGGTGTCAAACAGGAA | 2335 |
| Foxq1 | GAAGCCGGCTAAGAAACAAG | 2336 |
| Foxq1 | GAAGCTGGCGTGGTAGGCAT | 2337 |
| Foxs1 | GCTGCCCTGAGCCTGAGCTT | 2338 |
| Foxs1 | GTTCTGTCCTCAGGGCAGAC | 2339 |
| Foxs1 | GTACTGGGAGTTCTGTGAAC | 2340 |
| Foxs1 | GTACCAGCACCAATACCTAG | 2341 |
| Foxs1 | GCCTGAATAGTATAGCCCGG | 2342 |
| Foxs1 | GAAGGAAAGAGAGAGAGAGA | 2343 |
| Foxs1 | GAGAGTGGGAGACACAGCAG | 2344 |
| Foxs1 | GTAGACTTTGGAGGGCTACA | 2345 |
| Foxs1 | GATAGTTGTGTGGAGATGGG | 2346 |
| Gab1 | GAGTTGACTGATGTGATGCT | 2347 |
| Gab1 | GGTAGAACAGCTCCTGGGTC | 2348 |
| Gab1 | GGAGAATTCACCCTTCAAGA | 2349 |
| Gab1 | GTTCCTCTCTGGCTGCCTCG | 2350 |
| Gab1 | GTGTGTTTGAAACAAAGCCT | 2351 |
| Gab1 | GCTTGAGTGAGTTCTCCTCC | 2352 |
| Gab1 | GACCTCTTCCTTAAAGCATA | 2353 |
| Gab2 | GCTGGCTATTAATTCCTCTT | 2354 |
| Gab2 | GAGTTAACTTACAGTGAAGC | 2355 |
| Gab2 | GTAGATCAAAGGGTGCTTGG | 2356 |
| Gab2 | GCTTCGCTAGATTGTAATTT | 2357 |
| Gab2 | GAGGATTTGTGGCCAGCAGC | 2358 |
| Gab2 | GCGCTCTCCCATAGTGCCTC | 2359 |
| Gab2 | GCCATCTCCTCCACAAAGCC | 2360 |
| Gab2 | GGACTCATTTCAGCCAGAAT | 2361 |
| Gab2 | GCATGTCCTTGCAGGGCTTA | 2362 |
| Gab2 | GAGTGTGGCTTTGAATGTTA | 2363 |
| Gabpa | GATGGCGGAGTCTTAGCTGA | 2364 |
| Gabpa | GCCTCCTGGGACTGAGCTTC | 2365 |
| Gabpa | GGGTTGTGTGGCCTTTATCA | 2366 |
| Gabpa | GGATATCATAGAAGTGCGGT | 2367 |
| Gabpa | GCTGTGCTACAGTGCTTACG | 2368 |
| Gabpa | GAGTACGCTAAATTAGACGT | 2369 |
| Gabpa | GCGATCTGTTCATGATCACA | 2370 |
| Gabpa | GACAGAAGCCAAACAGGAGG | 2371 |
| Gabpa | GGACTCAGTGCAAGGTGACT | 2372 |
| Gabpa | GGTTTCTTCACGAGGAGAGA | 2373 |
| Gabpb1 | GAGACAACCGAGAATAACCT | 2374 |
| Gabpb1 | GGCCAGTAGGCTGATGTCTA | 2375 |
| Gabpb1 | GCAAAGGGAGAGGACTGCTA | 2376 |
| Gabpb1 | GTTCATTCCTTCAGCAGTGC | 2377 |
| Gabpb1 | GGTGAAGGCCATCCAGTCGA | 2378 |
| Gabpb1 | GTACCCGGAATCGCTGGTTG | 2379 |
| Gabpb1 | GGCATGGAGAAGCAGTAGTT | 2380 |
| Gabpb1 | GTTAGGTTTGGTTGGTTGGT | 2381 |
| Gabpbl | GATTTAACTTACCGTGGTCC | 2382 |
| Gata1 | GTGTATCTGAAGTTTGTTAC | 2383 |
| Gata1 | GAGGGCTAAATATAATCCCA | 2384 |
| Gata1 | GTCAGTCGGACCACTTAACA | 2385 |
| Gata1 | GTGATCTTATCCCAATCCTC | 2386 |
| Gata1 | GCCCTGGAAATCCGTAGGCT | 2387 |
| Gata1 | GCAAGGGTGAGAATTGGAGG | 2388 |
| Gata1 | GTGCCATTGGTGTGAGGATG | 2389 |
| Gata1 | GCCTAGCCTACGGATTTCCA | 2390 |
| Gata1 | GTGGAGGGACAATGGCTGGT | 2391 |
| Gata1 | GATCCTGATACTAATAGCAG | 2392 |
| Gata2 | GCAGTATGAGGCCCAGAACT | 2393 |
| Gata2 | GCGTCTGATGCGGGTCTGCT | 2394 |
| Gata2 | GTTCTTTGAATTTCTCAGAG | 2395 |
| Gata2 | GAGGTTCCTAAGATACCTTC | 2396 |
| Gata2 | GAATAACCGTTCTAATGAGG | 2397 |
| Gata2 | GACCACCAGGCTGAACTGCC | 2398 |
| Gata2 | GCTGAATAACCGTTCTAATG | 2399 |
| Gata2 | GTCGTCCGTAGCAGTGGAGG | 2400 |
| Gata2 | GGAGTCAGTTGGATTTGGGC | 2401 |
| Gata2 | GTCCGTAATTGGGTAACTGG | 2402 |
| Gata3 | GGTGCAGGGIGAACTCAGAA | 2403 |
| Gata3 | GGACGCCCGCGTTATTGTTA | 2404 |
| Gata3 | GGGCTTCCTCTTCCCTTGGC | 2405 |
| Gata3 | GTAGCAAAGCCGATTCATTC | 2406 |
| Gata3 | GGCACTGGATCCAGCCTGTA | 2407 |
| Gata3 | GGTCTGAGGTAGTTTAGGGT | 2408 |
| Gata3 | GCTTGGCTTTAGAGGGTTAC | 2409 |
| Gata3 | GTCTCTGGGACAGGGTCTGG | 2410 |
| Gata3 | GGGACACGATCCTCAGCACA | 2411 |
| Gata3 | GTTGCAGTTTCCTTGTGCTG | 2412 |
| Gata4 | GATTCTGACTGGCATTGTTT | 2413 |
| Gata4 | GCAGTCAGTCCTCGAACCTA | 2414 |
| Gata4 | GTCTCTAGGCACTGACCTTA | 2415 |
| Gata4 | GTTTCCAATACAAGATTAGA | 2416 |
| Gata4 | GAAAGCTACAGACTTAAGGC | 2417 |
| Gata4 | GGGAGGCCAAAGAGAGGAGG | 2418 |
| Gata4 | GAAGGAAAGCACTCAGTGCC | 2419 |
| Gata4 | GCACAGAGGTCGCCTAGTTC | 2420 |
| Gata4 | GCAACGCTGAGGATCAGACT | 2421 |
| Gata4 | GTGCCATCCCGAGCCTTCTC | 2422 |
| Gata5 | GATGGAGCTAGAAGGACCTA | 2423 |
| Gata5 | GTCAGTGCCCAGGTCTAGAC | 2424 |
| Gata5 | GGGAGCCTCAGCCAGTCTTT | 2425 |
| Gata5 | GTCCTCCAACTTGGCCACTC | 2426 |
| Gata5 | GCATTGACCAGTGGGCAGCA | 2427 |
| Gata5 | GGATTAAACTCAGTTCAGAT | 2428 |
| Gata5 | GGGTGCTGCAGACAGATACG | 2429 |
| Gata5 | GGACCGACTAGAAGAGAGAA | 2430 |
| Gata5 | GGGAGCTCGGAATAGACGTG | 2431 |
| Gata5 | GGGACCGACTAGAAGAGAGA | 2432 |
| Gata6 | GAAGGTGGCACACCAACCTA | 2433 |
| Gata6 | GATAACGCGTTGAGAAGGAG | 2434 |
| Gata6 | GTATATCACTGCTGCTGCCT | 2435 |
| Gata6 | GCTAAAGGACACCAAGGGAG | 2436 |
| Gata6 | GAACGGTTTATAGACCTACT | 2437 |
| Gata6 | GTTACAGCGCTGGATGATTA | 2438 |
| Gata6 | GGCGAGGTAGGGAATACACA | 2439 |
| Gata6 | GAGAAGGGAAATGACTTACT | 2440 |
| Gata6 | GTCAGTTACTAGCAACTGCG | 2441 |
| Gata6 | GACCTGAGCATCCCGAAACA | 2442 |
| Gbx1 | GCTTCCTCTCTCAAACACAG | 2443 |
| Gbx1 | GATTGATGAGGCCGGACCCG | 2444 |
| Gbx1 | GGACTCGGTTCTCTAAGCTC | 2445 |
| Gbx1 | GTAGCTAGTATCATGTGTTG | 2446 |
| Gbx1 | GAATACCTGCCCAATCAGAA | 2447 |
| Gbx1 | GACCTCACTGTAAATGGAGG | 2448 |
| Gbx1 | GAGGACAGAGCTGGGCTGAA | 2449 |
| Gbx1 | GGACAGTCAAGGCGGAATGG | 2450 |
| Gbx1 | GCTTTGTGAAAGTTTGCGGC | 2451 |
| Gbx1 | GAGCCAGGGAGATAGAGTGA | 2452 |
| Gbx2 | GATTGCGCCGAAAGGAAAGT | 2453 |
| Gbx2 | GCCTCTCATGAGGCCTCCAC | 2454 |
| Gbx2 | GAAGCTTCGGTCTGAGCAAG | 2455 |
| Gbx2 | GGCTGGCGGTGAAAGGGAAG | 2456 |
| Gbx2 | GAGCAGGGATCGCTCACGGA | 2457 |
| Gbx2 | GTATTATTATTACCTGGAGG | 2458 |
| Gbx2 | GAAAGGCACTGGCCAGCAGG | 2459 |
| Gbx2 | GTTGCGGCACACACTGTCCC | 2460 |
| Gbx2 | GTTATTTCCCAACTATGGCC | 2461 |
| Gbx2 | GCGGCTGAACTTCCCTGGTG | 2462 |
| Gcm1 | GTTATGAATGCCACAGAGAG | 2463 |
| Gcm1 | GAGCTTCAGACTCTTGGACT | 2464 |
| Gcm1 | GGTAAGGTCAGCTACTCCAC | 2465 |
| Gcm1 | GATGAGGCATGGCAGAACTG | 2466 |
| Gcm1 | GGAATCCCAGGTAGTCTGCT | 2467 |
| Gcm1 | GCTTTCAGCCAGGGACAGGT | 2468 |
| Gcm1 | GGAGTGTCTCTGCAAATTCA | 2469 |
| Gcm1 | GTCACCCATAGCATGCCTGA | 2470 |
| Gcm1 | GTCTAAAGGTGAGACTGGAA | 2471 |
| Gcm1 | GAGATGGCTTTCAGTGTTTC | 2472 |
| Gcm2 | GTCACTCTTAGACTCAGCGG | 2473 |
| Gcm2 | GCAGGTCACTGTTAGAGGAG | 2474 |
| Gcm2 | GCAAATCTGAAGGTTGGGAC | 2475 |
| Gcm2 | GGTTGAGGCTCTGGAAGCAA | 2476 |
| Gcm2 | GCTAAGGCTGACAATGGAAT | 2477 |
| Gcm2 | GCTGGTGCGCTTTACAGCCA | 2478 |
| Gcm2 | GTTTCCAACTTGGTCTGTTT | 2479 |
| Gcm2 | GAAGTTAAGCAGTAGGCAGT | 2480 |
| Gcm2 | GGTTTCCAACTTGGTCTGTT | 2481 |
| Gcm2 | GTGACCCAAACAGACCAAGT | 2482 |
| Gfi1 | GAAATGAGATCCTGGAGGAC | 2483 |
| Gfi1 | GTGAGCAGTTTAAGTGCTGC | 2484 |
| Gfi1 | GCGACGAACAGAAGCGAAAG | 2485 |
| Gfi1 | GCAGAAAGAAACCTGCGCCT | 2486 |
| Gfi1 | GGGAGCATAGGAGGAAGGCA | 2487 |
| Gfi1 | GCATAGGAGGAAGGCAGGGA | 2488 |
| Gfi1 | GAAGGCAGGGAAGGGAGAAG | 2489 |
| Gfi1 | GGACTATGTGCTGCAGTGGC | 2490 |
| Gfi1 | GACGAACAGAAGCGAAAGAG | 2491 |
| Gfi1 | GTTTCTCCTGGGACAAGTGT | 2492 |
| Gfi1b | GAGCATGGAACTTTGGAACA | 2493 |
| Gfi1b | GGTTTGGGTCAGGGCTGTAT | 2494 |
| Gfi1b | GGACTGGACCAGGAGTTCTC | 2495 |
| Gfi1b | GATGGATGCCTCCAGAGATG | 2496 |
| Gfi1b | GTTTGAGGCCTTTCTGCTGA | 2497 |
| Gfi1b | GATTGAATACCCAAGTACCA | 2498 |
| Gfi1b | GTGTAGCACAACCAGTTCAA | 2499 |
| Gfi1b | GAAATGCCCTGCGCTGGCCT | 2500 |
| Gfi1b | GAACATGGACCCAGATGTGG | 2501 |
| Gfi1b | GCTTTAGACAAGGAACCGGT | 2502 |
| Gli1 | GTCACAGTAGAAACAGATAA | 2503 |
| Gli1 | GTGCGACCTATGGAACATGA | 2504 |
| Gli1 | GTATAGGGTCCCTCAAGGGA | 2505 |
| Gli1 | GTGGTCCAGGGCTGGAAACT | 2506 |
| Gli1 | GGCAGTATAGGGTCCCTCAA | 2507 |
| Gli1 | GGTGACTGGACACAGAGAGA | 2508 |
| Gli1 | GGATATACGAGGGAAGTGAG | 2509 |
| Gli1 | GATATACGAGGGAAGTGAGC | 2510 |
| Gli1 | GGGTGGATAGAAGCTAGAGA | 2511 |
| Gli2 | GGTTTGTTTACATGTATTGG | 2512 |
| Gli2 | GGTAACAAGAAAGGAAGAAT | 2513 |
| Gli2 | GATCCAATCAGTGAGTAACA | 2514 |
| Gli2 | GGGTTTGTTTACATGTATTG | 2515 |
| Gli2 | GAAGGAGCTTTCTCTAAGGC | 2516 |
| Gli2 | GTCCACTCCAAGAAGCAAGC | 2517 |
| Gli2 | GAACAACCAGCGGAGGGCTG | 2518 |
| Gli2 | GCTACGGCGCACAGAGGATC | 2519 |
| Gli2 | GTAGCTGGAACTTTCTGGTA | 2520 |
| Gli3 | GGCCTGGATGTGTCTGTGTG | 2521 |
| Gli3 | GAAACATCCTTCACTCATCT | 2522 |
| Gli3 | GATACATTGITTCTGGCATT | 2523 |
| Gli3 | GTTATCTCTTAACTCAGTAG | 2524 |
| Gli3 | GGAAGTTTCAGGCTTGGCCT | 2525 |
| Gli3 | GAGAGGTGGGCAACTCAGAT | 2526 |
| Gli3 | GTTCTGATTTGGTTCAACCG | 2527 |
| Gli3 | GTTCTGATAGCGTGGTGGGA | 2528 |
| Gli3 | GAAACAAGAGGAATTCTTGA | 2529 |
| Gli3 | GTGAACATGGTTTACAGAAA | 2530 |
| Glis1 | GGAATGGGTACAGGAGAACG | 2531 |
| Glis1 | GCCTGAACTCTCCATTCAAT | 2532 |
| Glis1 | GGCCTGGGTTCAGATGACAA | 2533 |
| Glis1 | GGCAGCAGGGTCTCAACTGT | 2534 |
| Glis1 | GAAGACACTGGCGTGGGAGT | 2535 |
| Glis1 | GTCTGCTACAGCAGGTAGCC | 2536 |
| Glis1 | GTGTGTTTCTGCAACCGGCC | 2537 |
| Glis1 | GTGCAGGCGATGAGCTGTTA | 2538 |
| Glis1 | GGGTCCACACTTTAGAATTG | 2539 |
| Glis1 | GTAAATGAGTTTGTTGCTGT | 2540 |
| Glis2 | GTCCTGGATCTGGACTGGGC | 2541 |
| Glis2 | GATAATGTCGCAGTGCTGCC | 2542 |
| Glis2 | GGATAATGTCGCAGTGCTGC | 2543 |
| Glis2 | GCACATCGGTAGTGTGAAAC | 2544 |
| Glis2 | GTAGTTCAGCACGTGTTCCT | 2545 |
| Glis2 | GTGAGATACTGCACTAGGGC | 2546 |
| Glis2 | GGCCTTGTGCTTATTTCACC | 2547 |
| Glis2 | GCCTACCTTCGCACCAGACC | 2548 |
| Glis2 | GGTCCTGGATCTGGACTGGG | 2549 |
| Glis3 | GCTAAAGTTCCAAGCATCAC | 2550 |
| Glis3 | GGTAACTGGCATGAAGCTGA | 2551 |
| Glis3 | GACCACTGGAGTGTACAATG | 2552 |
| Glis3 | GCTGGAATAAATTCCATGTG | 2553 |
| Glis3 | GGACCTGTTGTCAACTCTCA | 2554 |
| Glis3 | GAAGGTGATGGCCAAAGGTA | 2555 |
| Glis3 | GGTACAGIACGAAGGCCAGG | 2556 |
| Glis3 | GTCAAGAGGGAGACACTGGC | 2557 |
| Glis3 | GGCAAGCTCTCTGAGGTAAC | 2558 |
| Glis3 | GCTAGCTAAAGTGAACAATG | 2559 |
| Gm4736 | GTGACTGAAGTACTATATAG | 2560 |
| Gm4736 | GGCCTACAAAGTCATCATGA | 2561 |
| Gm4736 | GGAAGGCCACAGTCTTTATA | 2562 |
| Gm4736 | GGCCACAGTCTTTATAAGGA | 2563 |
| Gmeb1 | GTCAGCTCGAAGGAGCACAT | 2564 |
| Gmeb1 | GGCAGTGAATGGAGCTTGTA | 2565 |
| Gmeb1 | GTGGAGTCCTCTCTGAGGCT | 2566 |
| Gmeb1 | GGTCAGCCACTGGGTTCAGC | 2567 |
| Gmeb1 | GAAAGCCTAGGTCAGCCACT | 2568 |
| Gmeb1 | GTGTGAGGAGGGAAGTAGGT | 2569 |
| Gmeb1 | GATGTCCTCTGTAAAGGATG | 2570 |
| Gmeb1 | GGCAATGTGGTCAGGCCTTG | 2571 |
| Gmeb1 | GTGGTGGAACTCAGGTGGTC | 2572 |
| Gmeb1 | GGTGGGTAAGTGCTCTGACA | 2573 |
| Gsc | GAACCTATCGGCACCCACGC | 2574 |
| Gsc | GTGAACAGCCTCTTCCTTCT | 2575 |
| Gsc | GTTTGCCAGGTGGCAATGTT | 2576 |
| GsC | GTTAGGAGCTAGGGAGAGTC | 2577 |
| Gsc | GCGCAGAACTAGGCAGTGCG | 2578 |
| GSc | GCCACTCAATATGTTGAGAA | 2579 |
| Gsc | GGGTCCGGGAGCTTCTTTCT | 2580 |
| Gsc | GATAGAGACCGGCTTCAGTT | 2581 |
| Gsc | GGAGAGATGCCAAGAGGAGG | 2582 |
| Gsc2 | GCCAAGTATTTGTTCTCAGT | 2583 |
| Gsc2 | GCAGCCATTCTGTAACCATG | 2584 |
| Gsc2 | GAGGGAATGAGGGAAGCCAG | 2585 |
| Gsc2 | GGAGGGAATGAGGGAAGCCA | 2586 |
| Gsc2 | GCCAGGCTCTGTGCACTTGG | 2587 |
| Gsc2 | GAAGCCCATAGAGTCCTCAC | 2588 |
| Gsc2 | GCACCATGTCATCTTCCTAC | 2589 |
| Gsc2 | GGACTTGGTAAAGTGGGAGA | 2590 |
| Gsc2 | GGGATTAGCACGCGCGAACG | 2591 |
| Gsc2 | GGGATTAGCACGCGCGAACG | 2592 |
| Gsx1 | GAGCAATTAGAACGGGAATT | 2593 |
| Gsx1 | GGGAGTGAGAGCCGAATTCG | 2594 |
| Gsx1 | GTTGCCAGCGCCTTCTCTTC | 2595 |
| Gsx1 | GGAACGCAGAGGCAGAAGGC | 2596 |
| Gsx1 | GAAGCTGTGTACACAGAGCG | 2597 |
| Gsx1 | GAGAGAAGAGACTCCACAGG | 2598 |
| Gsx1 | GATCGCCAGCGCAAAGCCAA | 2599 |
| Gsx1 | GTAACAGAAAGAAAGGGACC | 2600 |
| Gsx1 | GGAAGAAGTAACAGAAAGAA | 2601 |
| Gsx1 | GAGTGCACCGGCGTGTCTAG | 2602 |
| Gsx2 | GCCGAATAAATCCTTCCACG | 2603 |
| GsX2 | GAGGGAGAAGACAGATATAG | 2604 |
| Gsx2 | GAGCTCTAATTGCCAGGACT | 2605 |
| Gsx2 | GTGGTCACAGAGATGGAAAG | 2606 |
| Gsx2 | GGGCAGGGAACAGCAGTTGG | 2607 |
| Gsx2 | GAGAGTGATGGAGGGAGAGG | 2608 |
| Gsx2 | GCCTACCTTCCTCCCTCGCT | 2609 |
| Gsx2 | GAGAGTAGGTTGGTCGGAGC | 2610 |
| Gsx2 | GCTGGTTAGAAAGATGCACA | 2611 |
| Gsx2 | GGTAGGTTATCTACAGTCCT | 2612 |
| Gft2a2 | GCGTGAAAGGCTTCAGTGTG | 2613 |
| Gtf2a2 | GGTTGGTATCAGTCTCCACC | 2614 |
| Gtf2a2 | GACTGCAGTGTAGGGAAACC | 2615 |
| Gtf2a2 | GCAGCTATAGGTACTGCAGA | 2616 |
| Gtf2a2 | GCAAGAGGTGCCAGGAAGTG | 2617 |
| Gtf2a2 | GTTTACCAGCCGTGAAGGGT | 2618 |
| Gtf2a2 | GAGCCAAAGTATAACAGAGA | 2619 |
| Gtf2a2 | GTCTATATACAAAGGTACCA | 2620 |
| Gtf2a2 | GGTAGCTGTCAGTTACTCCA | 2621 |
| Gtf2a2 | GAAACAGATCACGTATGGTG | 2622 |
| Gtf2f2 | GTCCTGACGTAGTCGTGCGC | 2623 |
| Gtf2f2 | GTTTGAAAGAGGCTCTGAAA | 2624 |
| Gtf2f2 | GTGTAAAGATCAGGGAAAGC | 2625 |
| Gtf2f2 | GCAGGTGGATGGGCTTGGTG | 2626 |
| Gtf2f2 | GCATCACACACTATCATATG | 2627 |
| Gtf2f2 | GCAGTAAGGTATTGGAAGAA | 2628 |
| Gtf2f2 | GAACCGTGCGTTTACAGCAA | 2629 |
| Gtf2h1 | GGTGGAAGCAAGAAGGCACG | 2630 |
| Gtf2h1 | GCCCAGTATGTAAAGATCTT | 2631 |
| Gtf2h1 | GATGACAGGTGGAAGCAAGA | 2632 |
| Gtf2h1 | GTTCAGGATAGCTGAATAAT | 2633 |
| Gtf2h1 | GTTCTTCCGCTGGGAGGGAC | 2634 |
| Gtf2h1 | GCCTTCGGGCAGTAGATTAA | 2635 |
| Gtf2h1 | GCCAGCGTTTGTTAGGAGGG | 2636 |
| Gtf2h1 | GCCTCACTTCCTTCGTTCTC | 2637 |
| Gtf2h1 | GGTAAGTTGAGACCGAAGAA | 2638 |
| Gtf2h1 | GGCGTGATCGTCACGTGACG | 2639 |
| Gtf2h2 | GCCAGGCTTGCTCTTTGCTT | 2640 |
| Gtf2h2 | GTTCTCTTGAACACAAGGAA | 2641 |
| Gtf2h2 | GACAGATCACCTCCCACATG | 2642 |
| Gtf2h2 | GAGGGCAACTACGTATGGTG | 2643 |
| Gtf2h2 | GACTGCCGGTACTTCCGGTG | 2644 |
| Gtf2h2 | GTTCACCAATATTTCTGCTG | 2645 |
| Gtf2h2 | GTGTAGACAAGTGTGAGACC | 2646 |
| Gtf2h2 | GGGAGGTGATCTGTCCTGCC | 2647 |
| Gtf2h2 | GCTGCCAGAAGAGGGAGCTA | 2648 |
| Gtf2i | GGCCTGCTGGAGAAGGAAGG | 2649 |
| Gtf2i | GTTCATGCCGCAAGGCTGTC | 2650 |
| Gtf2i | GGGTTCAGAACTACAACTCC | 2651 |
| Gtf2i | GTGGCCTGCTGGAGAAGGAA | 2652 |
| Gtf2i | GTTTACTTTCTTTGTAGCTG | 2653 |
| Gtf2i | GTAAACTTAAGACCCTCCTC | 2654 |
| Gtf2i | GAGGGCGCCCGAATATTCGG | 2655 |
| Gtf2i | GGCGGACATAAGCGGTGGGA | 2656 |
| Gtf2i | GGACAGGCAACGGATGGGAG | 2657 |
| Gtf2i | GTCGCCTGATTTGCAGAGGG | 2658 |
| Gtf2ird1 | GGGATCAGAAACAAGGCCAT | 2659 |
| Gtf2ird1 | GTAGCTGGCAGAGAGGCTAT | 2660 |
| Gtf2ird1 | GGACAGGATCAGTAGAGGGA | 2661 |
| Gtf2ird1 | GGCTAGGCCTTTGCTGGGAT | 2662 |
| Gtf2ird1 | GTGTCCAAGGTCAGAAGGGA | 2663 |
| Gtf2ird1 | GATGAGGGATGATGGAGATG | 2664 |
| Gtf2ird1 | GTAGAGGGAGGGAGGGAAGG | 2665 |
| Gtf2ird1 | GGGACAGGATCAGTAGAGGG | 2666 |
| Gtf2ird1 | GTAGTATACAGGAGGTCAGA | 2667 |
| Gtf2ird1 | GATCTAGAAGGAGACCAGGT | 2668 |
| Gzf1 | GGTAAAGCAATGATTTACCG | 2669 |
| Gzf1 | GGTGGGTCAAGTCTTGGCGT | 2670 |
| Gzf1 | GCAGAGCTATTTGACAAAGT | 2671 |
| Gzf1 | GTGTGAGGGACAAAGCGCTG | 2672 |
| Gzf1 | GTGTTATGGAGCCAACCACA | 2673 |
| Gzf1 | GCCTGAGTCTCCCAGTGTGA | 2674 |
| Gzf1 | GGAGACTCAGGCAGCCACTG | 2675 |
| Gzf1 | GCTAAGGCGCAACCAAAGGA | 2676 |
| Gzf1 | GTGGGTCAAGTCTTGGCGTA | 2677 |
| Hand1 | GAGGTGGAAGTGGGAGGGAA | 2678 |
| Hand1 | GTAACTTAGGAGACTGAAGC | 2679 |
| Hand1 | GTTGTGCAAGAGATTGTGAG | 2680 |
| Hand1 | GTGTAAGACAATTACCAGGC | 2681 |
| Hand1 | GTTCAGTACAGGGAGTGAGC | 2682 |
| Hand1 | GAAGTGGGAGGGAAAGGGAG | 2683 |
| Hand1 | GTGAGTGTCCATTGTCCTTG | 2684 |
| Hand1 | GTGATCTGGGATCTCAGGCA | 2685 |
| Hand1 | GGGCACTGACCAGTTTGTTC | 2686 |
| Handl | GTGGGAGCCTGAAGGCCATT | 2687 |
| Hand2 | GCCAGGTAAACTTGCTGCTT | 2688 |
| Hand2 | GCTTGTACAGCCCAAGAGTG | 2689 |
| Hand2 | GGCTGTACAAGCAGGCCCTC | 2690 |
| Hand2 | GTCTGGAAGGCCACATCAGA | 2691 |
| Hand2 | GTAGCTGGACCTAGTCTTGC | 2692 |
| Hand2 | GGACCTGAGGAGGCAAGCAG | 2693 |
| Hand2 | GTACCCTGGGAGCAAGAAGA | 2694 |
| Hand2 | GAAGAAGGTCCCTGTGTAAT | 2695 |
| Hand2 | GTGCTGTCAGTGAGGAGTGA | 2696 |
| Hand2 | GTGATTATGAGGGAACTAAC | 2697 |
| Hbp1 | GTTGCATCATCAAAGATTTG | 2698 |
| Hbp1 | GTATCTGAAAGTTGTACACT | 2699 |
| Hbp1 | GGTGCTGAAATACCCAACCA | 2700 |
| Hbp1 | GTTTCTCTTTCTACTTTGTT | 2701 |
| Hbp1 | GGCCTAGAGCGTCCTTGGTT | 2702 |
| Hbp1 | GTTGGCGGCGTATTGAGTCA | 2703 |
| Hbp1 | GCCAAGTGCCATGTACTGTA | 2704 |
| Hbp1 | GGCTGTGTCTCAACTAATTC | 2705 |
| Hdac2 | GTTGGACACAGTTTCACAAG | 2706 |
| Hdac2 | GGAAGAAGACTAGCATGAGT | 2707 |
| Hdac2 | GGGAAGAAGACTAGCATGAG | 2708 |
| Hdac2 | GAGTAATTCTAAGTCTCTTG | 2709 |
| Hdac2 | GGTTGGGTCAGGGACCACAG | 2710 |
| Hdac2 | GTGTTTATTACGAGCAGGTA | 2711 |
| Hdac2 | GATAAAGTAGACAAAGCACG | 2712 |
| Hdac2 | GGAGTAATTCTAAGTCtCTT | 2713 |
| Hdac2 | GGTAGCGGGTGTGTGTGTGG | 2714 |
| Hdx | GCACTTATCTGCTAAATCTG | 2715 |
| Hdx | GCAATCACCTGTGAATTACA | 2716 |
| Hdx | GGAAGAGGCAGCCCTACTAC | 2717 |
| Hdx | GGACCCAGTTTGAGCACACT | 2718 |
| Hdx | GTTGTACACTTACTTTGTTC | 2719 |
| Hdx | GAATATGGCAAAGTGAAAGA | 2720 |
| Hdx | GTAGTAGGGCTGCCTCTTCC | 2721 |
| Hdx | GAAAGCAAAGTACAAATTGT | 2722 |
| Helt | GGGAGAGCTTCTGGAGACGG | 2723 |
| Helt | GCTGTGAGATGCAGGACTTC | 2724 |
| Helt | GGATGTCCGGACAAATAAAG | 2725 |
| Helt | GTTAGACAGTGAGACTGGGT | 2726 |
| Helt | GCAGCACCTAGGAAGCTCCG | 2727 |
| Helt | GTAAATCACCCGGAGATCCA | 2728 |
| Helt | GTATATTCACTCGCACACAA | 2729 |
| Helt | GTGCCTGGAGGGTGTGGAAT | 2730 |
| Helt | GGTATATTCACTCGCACACA | 2731 |
| Helt | GAAGTTGATCCTCTTACTGT | 2732 |
| Hes1 | GGCTTTCTGGACAATGCTTG | 2733 |
| Hes1 | GTTCTATAACTGAGGACATC | 2734 |
| Hes1 | GAGAGGAAGGGAGCTACCGA | 2735 |
| Hes1 | GCAGTTTGACATCAGCCGGC | 2736 |
| Hes1 | GCTGATGTCAAACTGCAGCT | 2737 |
| Hes1 | GATATATATAGAGGCCGCCA | 2738 |
| Hes1 | GAGAGGAATGAATGGGCTAG | 2739 |
| Hes1 | GTAAGGGCATGTTTAGCGTG | 2740 |
| Hes1 | GGCTCCTAAGTGGCACAGGT | 2741 |
| Hes1 | GCTTCTAGTAGGGCTACTGG | 2742 |
| Hes2 | GGTTGTTCGGGTCTCGCCTT | 2743 |
| Hes2 | GTGCTTGAGGAGCGGAGCCA | 2744 |
| Hes2 | GTCTTTGATCAGTGTAGGGT | 2745 |
| Hes2 | GCTTGTACAAAGTAACTCCT | 2746 |
| Hes2 | GCTCCATTGAGGGCTTTGGT | 2747 |
| Hes2 | GTCACATGACAGACGAGTGG | 2748 |
| Hes2 | GGGACACTGGACTGAGTTGG | 2749 |
| Hes2 | GATCAGTGTAGGGTGGGCTT | 2750 |
| Hes2 | GCGTCTGTCAGGAGCCTTTC | 2751 |
| Hes3 | GACAGACTCATCACTGCCCT | 2752 |
| Hes3 | GCACAAACTGGTATGGGTGC | 2753 |
| Hes3 | GAAGCCCTGAAATGACTCAG | 2754 |
| Hes3 | GACTGGGACGAGAGCTTCCT | 2755 |
| Hes3 | GGCTTCTTCCCTTCCCGCTC | 2756 |
| Hes3 | GTGTGGTTTGACAGGGAGCA | 2757 |
| Hes3 | GGGATACAGTCACACAGAGA | 2758 |
| Hes3 | GAGCTCCGAGGAATTCTAAG | 2759 |
| Hes3 | GCTCAGTGGTTAGCACATTC | 2760 |
| Hes3 | GAAACCCTGCTTATGCAAAC | 2761 |
| Hesx1 | GAGAGATACACGTTTACATG | 2762 |
| Hesx1 | GCAACAGGGACTGAGCGAGC | 2763 |
| Hesx1 | GAATATGAGAGTGCAAGTGG | 2764 |
| Hesx1 | GGCATTTGACAAAGCTTTGC | 2765 |
| Hesx1 | GCACTCTGTGTTAATAACAC | 2766 |
| Hey1 | GTGGATGGAGAACTGGACCT | 2767 |
| Hey1 | GTCATCTGCAGCTCAGAAAG | 2768 |
| Hey1 | GGGATTGCAGGCTCCAAGAG | 2769 |
| Hey1 | GTGATATGAGGCTCTGAAGA | 2770 |
| Hey1 | GCAGATTGGCAGCCGCATGG | 2771 |
| Hey1 | GTGTTAGATGGAGATGTAAT | 2772 |
| Hey1 | GATAAGGAGAAGGGAGAGAA | 2773 |
| Hey1 | GCACCTTCTGATAAGGAGAA | 2774 |
| Hey1 | GAAACATGGGATGGCGTCAA | 2775 |
| Hey1 | GGGTGCTCCGTCACTTTAGG | 2776 |
| Hey2 | GCACACACCGGAGAAACTGG | 2777 |
| Hey2 | GTGAGCGTGTGTGACGTCTA | 2778 |
| Hey2 | GACACAGAAACTGGAGGGAG | 2779 |
| Hey2 | GGCTGTCTGCTCTGTCCCTG | 2780 |
| Hey2 | GAGTTCAAAGTTCTCGGATT | 2781 |
| Hey2 | GCGTGTGTGACGTCTAGGGT | 2782 |
| Hey2 | GGTGTGTTTAGACAGGAGAC | 2783 |
| Hey2 | GCTGCACACACCGGAGAAAC | 2784 |
| Hey2 | GACTGGACTGGGCGCAGATT | 2785 |
| Hey2 | GCAAATCACAGGATCATCGG | 2786 |
| Heyl | GAAATGCCTAGTGCACACAT | 2787 |
| Heyl | GGCAGGGAGATGGTGGAGGT | 2788 |
| Heyl | GTGCTATGCTGTCAGTTCAG | 2789 |
| Heyl | GGCAGAAGAAGAAGGAGAGC | 2790 |
| Heyl | GACTGAAGAATTACTTCCAA | 2791 |
| Heyl | GAGCCTTCGGCTTCTCTTTC | 2792 |
| Heyl | GGCACCAGGGAGAGGAAGAG | 2793 |
| Heyl | GTAGGGTGTGGTGGTTGGTG | 2794 |
| Heyl | GTGCAGAGGGAAGCTGAGGG | 2795 |
| Hhex | GAGTTGGGCAGTTTCTGCTA | 2796 |
| Hhex | GAGAAGCGATGGGACTCTGC | 2797 |
| Hhex | GACTGCGACCGTCGAAGAGG | 2798 |
| Hhex | GCAGTGTTCTTCGATCCAAT | 2799 |
| Hhex | GGCTTAGTAGTAAGGGTTAC | 2800 |
| Hhex | GTGAACTACTGGAAGGTTGC | 2801 |
| Hhex | GGCCAGAAGGCTGCGCTTCT | 2802 |
| Hhex | GATTCCGTTAGCATCCAGGG | 2803 |
| Hhex | GAATCTGAAGCCAGCGCCAT | 2804 |
| Hhex | GTTCGTTTCCTGCTTCCACC | 2805 |
| Hic1 | GACCGGCAAGACAGACCGAC | 2806 |
| Hic1 | GTGTCTTCCCTAGAGGACTC | 2807 |
| Hic1 | GTGTGGAGCATGCAGGACGG | 2808 |
| Hic1 | GGGCTCAATAGCTTGGCAGA | 2809 |
| Hic1 | GTGGTATCCTCGCTCTCTCC | 2810 |
| Hic1 | GGGACTCCGGAGTGAGGATG | 2811 |
| Hic1 | GCTCTCTCCTGGTGTGTGTG | 2812 |
| Hic1 | GAGTGAATAAACACAGAACG | 2813 |
| Hic1 | GTAAGTGGATTAGATGGAGG | 2814 |
| Hic1 | GACCACCAACAGTCGGAGAT | 2815 |
| Hif1a | GCCATAAATAGATACCACCA | 2816 |
| Hif1a | GCAGTCCTGTCAAGGTCTGT | 2817 |
| Hif1a | GACACAACTGAGTCTGAATC | 2818 |
| Hif1a | GTAAGGTCTGCAAAGTGAGT | 2819 |
| Hif1a | GGCACTTTAACAGTTGAAAC | 2820 |
| Hif1a | GCTGAGAGCAACGTGGGCTG | 2821 |
| Hif1a | GCTCTCAGCCAATCAGGAGG | 2822 |
| Hif1a | GTTGCTCTCAGCCAATCAGG | 2823 |
| Hif1a | GTTGTGCAGATTGTGAAATG | 2824 |
| Hira | GCGCATTTATTAGAAGAGCG | 2825 |
| Hira | GTGTCTGACGTGTGCCTGGC | 2826 |
| Hira | GGAACTTTGGATGCTTTCTT | 2827 |
| Hira | GGTCTGGGATTCCGAGAGGC | 2828 |
| Hira | GAAATCTGCTTGCTAACCCA | 2829 |
| Hira | GAAGTGAACGTGCTGAACTA | 2830 |
| Hira | GGGTGATGCTGTGTGCTGCG | 2831 |
| Hira | GTCTGCCGCTAGATGCATGC | 2832 |
| Hira | GTCCACTGTCTTCCCGAGGA | 2833 |
| Hira | GGGCGCATTTATTAGAAGAG | 2834 |
| Hivep1 | GGGCGTGAGAGGAAACGCTG | 2835 |
| Hivep1 | GGGCTGGGTTGTTGACTTGG | 2836 |
| Hivep1 | GTGGGCGTGAGAGGAAACGC | 2837 |
| Hivep1 | GCTTAGGCTCTGGGAAGCAC | 2838 |
| Hivep1 | GGTTCAAACAGCTCGGCTGG | 2839 |
| Hivep1 | GCTTGGCTTGGGAAGAGCCC | 2840 |
| Hivep1 | GAACTTTGGAAGCCGAAGAG | 2841 |
| Hivep1 | GGAACTTTGGAAGCCGAAGA | 2842 |
| Hivep1 | GGAATAACCTTGGCTTTCCT | 2843 |
| Hivep1 | GAGAGCATCGGTCCAACCCG | 2844 |
| Hivep2 | GAAGTTCTCTGATCCTACAA | 2845 |
| Hivep2 | GACTCGCCAGTGTTTCTGCG | 2846 |
| Hivep2 | GAACGCTCGAATCCAAAGAG | 2847 |
| Hivep2 | GAAGGGAATCCCAAGCGAGT | 2848 |
| Hivep2 | GCGAGAAATCCTTGGTACGC | 2849 |
| Hivep2 | GGCTAGAGAGGGAAGGGAAT | 2850 |
| Hivep2 | GAGAACCAGAAGCGCGCAGC | 2851 |
| Hivep2 | GGACTCGTGTGCACCCTCAA | 2852 |
| Hivep3 | GTGGGCTTCAGAGTGCATGA | 2853 |
| Hivep3 | GGAGAAACATATGCAAATAC | 2854 |
| Hivep3 | GTTGGATCAGAATGAGGTCA | 2855 |
| Hivep3 | GCGGTCTTGACGTTGAGCGC | 2856 |
| Hivep3 | GAACCTCCAACTTAACCTCT | 2857 |
| Hivep3 | GGGATTAAGCTGGAGGTGGA | 2858 |
| Hivep3 | GTAGTTGGCATGCACAGTTT | 2859 |
| Hivep3 | GTGATGGAGGAGCCTGCTGA | 2860 |
| Hivep3 | GTATTGGAGAATAGCAGCCT | 2861 |
| Hivep3 | GGATCCCTAGCTATTGAAAG | 2862 |
| Hltf | GTCAGACGCTCCCTATCTGA | 2863 |
| Hltf | GGCTTCTTGAGTGAGCCACA | 2864 |
| Hltf | GCTCAAGGTTCTGACGGACT | 2865 |
| Hltf | GTATGCGAGACCCTGAGTTC | 2866 |
| Hltf | GCTAAGAATAAATAGAGTCA | 2867 |
| Hltf | GTTCACGAGGTGAAGGGCTG | 2868 |
| Hltf | GAGGCACCAATGCATTGTCG | 2869 |
| Hltf | GAAATGCAGGTATCCCACCC | 2870 |
| Hltf | GTAAGGTCCGAGGTGGTGGC | 2871 |
| Hltf | GTGGTGTGGACACGTCTCAC | 2872 |
| Hlx | GATGTCCCAGTATCAGGGAC | 2873 |
| Hlx | GCTATGATGTCCCAGTATCA | 2874 |
| Hlx | GGCTACTATCAGCTCAGGAT | 2875 |
| Hlx | GTAGACTTGGGTCGGGATTC | 2876 |
| Hlx | GGCTATGATGTCCCAGTATC | 2877 |
| Hlx | GTCTAGCAGGGAGCAGAGGG | 2878 |
| Hlx | GCCTGTGGTCTGTTTGGGAG | 2879 |
| Hlx | GGGAGCTCCGATTAGGCCTC | 2880 |
| Hlx | GCCAAAGCGACTGGTCTACA | 2881 |
| Hlx | GTTGCGTTGTGCACCTAGTC | 2882 |
| Hmbox1 | GTCTAGCATCCATGGTATTC | 2883 |
| Hmbox1 | GCTGGAAGCTGTAGTTCCCT | 2884 |
| Hmbox1 | GATGGAAAGGAAGGATGAAT | 2885 |
| Hmbox1 | GCGGCGGCGATGAATTTGAG | 2886 |
| Hmbox1 | GACTTTCACAGGTGCACATG | 2887 |
| Hmbox1 | GTTTCCACTACTAAGTCAGA | 2888 |
| Hmbox1 | GTTTATTCAAACCCTTTGGT | 2889 |
| Hmbox1 | GAAGACCTCCTGACAGATGC | 2890 |
| Hmbox1 | GAATCTTCCTAATTGCTACG | 2891 |
| Hmga1 | GTGTTTGCCTACTTCTAGAG | 2892 |
| Hmga1 | GGATACCCTTCCTTCCTGGA | 2893 |
| Hmga1 | GGCGGCCCTGCTGTTTAAGT | 2894 |
| Hmga1 | GGTTCGAGTTTCCCGCCTCT | 2895 |
| Hmga1 | GAGATCCCAACTGGAATGTC | 2896 |
| Hmga1 | GGGCACAAAGATGGAGGGCG | 2897 |
| Hmga1 | GCTCCTTTGAAGCCTGCACC | 2898 |
| Hmga1 | GTTGCAAGGAAGTCCTGTTC | 2899 |
| Hmga1 | GTAGGAGATGCAGGAAGCAC | 2900 |
| Hmga1 | GAAGACCAGACAAGAGGCAG | 2901 |
| Hmga2 | GAAGTTTCCGGAAGCATTCA | 2902 |
| Hmga2 | GAGTTCTGAGTCTTCTCATT | 2903 |
| Hmga2 | GTTATGGGCGTCCCAGCACG | 2904 |
| Hmga2 | GGCATTTCTCAGTGGAGCGG | 2905 |
| Hmga2 | GTGCACGCTTGTTTGTGCGC | 2906 |
| Hmga2 | GACAGCAGGTGAAGGAGAAA | 2907 |
| Hmga2 | GCTTGGAGAGGGAAGAGACT | 2908 |
| Hmga2 | GCGGCACTGCACAGATGCAG | 2909 |
| Hmga2 | GCACCCAAATTTATAAAGCA | 2910 |
| Hmga2 | GGTAGAAGCCAAGCTCTCCA | 2911 |
| Hmx1 | GAAGTCTGGGTTACCCTCTG | 2912 |
| Hmx1 | GATGGAATGCTCTCATATCC | 2913 |
| Hmx1 | GGATAGGTGAGACAGAAACA | 2914 |
| Hmx1 | GCTTGGGAGCACTAGAAAGA | 2915 |
| Hmx1 | GTCTTACCCAGCACTCCCTC | 2916 |
| Hmx1 | GGACCAGGCAGACTCTGCTA | 2917 |
| Hmx1 | GGAGAGCCTTGCTCACCCTC | 2918 |
| Hmx1 | GATCCAATCGCGCAGATTTA | 2919 |
| Hmx1 | GATCTGTCAGGAAACCTGCC | 2920 |
| Hmx1 | GTTGCCTTCTCCTGGACAGT | 2921 |
| Hmx2 | GATCAGGTAACAGGTGCTCT | 2922 |
| Hmx2 | GAGAGCACTGACTGGTGTTG | 2923 |
| Hmx2 | GCGACACTAAGAAGTTTGCC | 2924 |
| Hmx2 | GGGAGTGAAGTTTGGTCACG | 2925 |
| Hmx2 | GTGTGGGAAGGCGAGCTGTG | 2926 |
| Hmx2 | GCATCCTGAAACAGAAAGCC | 2927 |
| Hmx2 | GGAGTCTGAAAGAGGAGGTG | 2928 |
| Hmx2 | GTCACCGCATTAACCTCTTC | 2929 |
| Hmx2 | GGAGCTTTGCTGCTCTGGGC | 2930 |
| Hmx2 | GGGAGAGGCCACAAGAAGGA | 2931 |
| Hmx3 | GCCGAGATICICCAGGGACT | 2932 |
| Hmx3 | GAAAGATAAAGAACGGGCTG | 2933 |
| Hmx3 | GATTTCGTATAAGGCTTTAC | 2934 |
| Hmx3 | GCTACTTACAAGGCAATAGT | 2935 |
| Hmx3 | GCGGGCCTCTGAGGAATAGC | 2936 |
| Hmx3 | GCCGGAAATCAGACCATAAA | 2937 |
| Hmx3 | GGAGAGAACTCTTCCAAAGG | 2938 |
| Hmx3 | GGCCAAGGAACTATCACCAG | 2939 |
| Hmx3 | GCTGCCTCTTAACTCTTCTT | 2940 |
| Hmx3 | GACACCTGCAGCATGTCCCA | 2941 |
| Hnf1a | GCTGGGACAGCAGGAAGCTC | 2942 |
| Hnf1a | GCTAGAGACCTGCATAGGAA | 2943 |
| Hnf1a | GGGAGTCATGGCCTGCAATT | 2944 |
| Hnf1a | GTGGTTGGTGGCACGATTGT | 2945 |
| Hnf1a | GCCTGTTTCTTTGGGCCGCT | 2946 |
| Hnf1a | GAGTGAGCAGAAGGGAGGGT | 2947 |
| Hnf1a | GCAATTGGGAGTGAGCAGAA | 2948 |
| Hnf1a | GCCCAACATCAGACTTCCCA | 2949 |
| Hnf1a | GGCAGTTTCCAGAATCTTCA | 2950 |
| Hnf1b | GATCACCTGTGGGAGGACTC | 2951 |
| Hnf1b | GCAGTAACTCCTCCAAGGCC | 2952 |
| Hnf1b | GAAGACCACCTGTGCAAAGC | 2953 |
| Hnf1b | GGAGCCGACTTAGGGAAGCC | 2954 |
| Hnf1b | GTGCCTCCTTGCTTCCTCTC | 2955 |
| Hnf1b | GGACGGCAGTAACTCCTCCA | 2956 |
| Hnf1b | GAACCTAAGGGACAGTCCAA | 2957 |
| Hnf1b | GTCTGAAAGCTAAAGGGTGG | 2958 |
| Hnf1b | GCTCTGGCAAGTCCCAATCC | 2959 |
| Hnf1b | GTTTGGCTGATAAACAGAAT | 2960 |
| Hnf4a | GGGTGCCTGCCTTGGAAGAT | 2961 |
| Hnf4a | GAAAGACCCAAGTGTGGGCT | 2962 |
| Hnf4a | GAGAACCACAAATCCACTTG | 2963 |
| Hnf4a | GCAGGACCTTAGGAAGCTTC | 2964 |
| Hnf4a | GTGAGTTTAGAAACTCTCTG | 2965 |
| Hnf4a | GACTATTAATGAGCGGGAGG | 2966 |
| Hnf4a | GTTGGTTTCTGACTGACACC | 2967 |
| Hnf4a | GTCCTCTGGGAGACTCAGCC | 2968 |
| Hnf4a | GACTCCCACTAGCTGGAGAA | 2969 |
| Hnf4g | GACATATTGTTGGACTTGAA | 2970 |
| Hnf4g | GGCTGTAAACAGCACACCTG | 2971 |
| Hnf4g | GGGTAAGAACATTAAGGGAG | 2972 |
| Hnf4g | GACATGCCAATGTTGCAGAG | 2973 |
| Hnf4g | GATTTCCATCATATGATCAT | 2974 |
| Hnf4g | GCCTAAGAGATCCAGATGAA | 2975 |
| Hnf4g | GCATCTGCAGTCCTGCTCCC | 2976 |
| Hnf4g | GATCCTCTGAGAGCTTTCTG | 2977 |
| Hnf4g | GTGTTGCAGTCACTGAGGGA | 2978 |
| HnF4g | GCTTTGTTCTGCAAGAGTTC | 2979 |
| Homez | GGGAACCAAACACCTGACTC | 2980 |
| Homez | GGGAAGAGTCTGTGCTTGAA | 2981 |
| Homez | GAGATCTGAAGGTGACCTCT | 2982 |
| Homez | GCCAATC3CGGACCTCTGCT | 2983 |
| Homez | GGAAGGAGATCCACACAATT | 2984 |
| Homez | GAGTTCGTGAAATGAGGAAA | 2985 |
| Homez | GTCTTCCGAGGGCCTTCCTG | 2986 |
| Homez | GCTCTTCTGATTAATGGACT | 2987 |
| Homez | GGGCTGGGAACATGTCTTCC | 2988 |
| Homez | GGAAGGACCACAGGATGCAG | 2989 |
| Hoxa1 | GAGGCCTCCTGGCTCTCTTG | 2990 |
| Hoxa1 | GTAATTTACGTGTGAGTTTG | 2991 |
| Hoxa1 | GTCCCTCTACATTCCGAGGC | 2992 |
| Hoxa1 | GGTGAAGAAAGAGGGCTTGG | 2993 |
| Hoxa1 | GCCTCCTGGCTCTCTTGTGG | 2994 |
| Hoxa1 | GAGCATGCTCACTCTAAAGT | 2995 |
| Hoxa1 | GAGCCTCCTCGGGAAAGCTT | 2996 |
| Hoxa1 | GCAGAGGATTATTTCACTCA | 2997 |
| Hoxa1 | GGGAGGGACAGATGACTGAG | 2998 |
| Hoxa1 | GTGGATGGGACCCTTTCCAA | 2999 |
| Hoxa10 | GAACTGTGGTTTGGGAGGTC | 3000 |
| Hoxa10 | GCTGCCTCAAAGTGGAGGTT | 3001 |
| Hoxa10 | GATGAGGAAGTCCATTCCCT | 3002 |
| Hoxa10 | GACCAGCAATAGAAGCCTGA | 3003 |
| Hoxa10 | GTGTGAGATCCAGACAGGGA | 3004 |
| Haxa10 | GAGCGAGAGAGAAAGCAGTG | 3005 |
| Hoxa10 | GATAGCACTCTGAGAGGGAG | 3006 |
| Hoxa10 | GCAAAGAGTGAGAGGGCGAT | 3007 |
| Hoxa10 | GCCAGATCTCTCATGCTGAA | 3008 |
| Hoxa10 | GGAATGAGGGATTTGGGAGG | 3009 |
| Hoxa11 | GAAGAAAGGGAGGTCTCTGA | 3010 |
| Hoxa11 | GCTACTATTGAGCAGCCTTA | 3011 |
| Hoxa11 | GCTTTGCCTGTTGGCGGTTT | 3012 |
| Hoxa11 | GTGTGCTCTTATCCCTAGTT | 3013 |
| Hoxa11 | GGCTGACAGAGCAATTCGAC | 3014 |
| Hoxa11 | GAAGCCGCCTCTTCTAGAAA | 3015 |
| Hoxa11 | GTGGGTGAGGGATACTCTCT | 3016 |
| Hoxa11 | GAAAGGAAGCCGAGGAGGGA | 3017 |
| Hoxa11 | GAGAGTATCCCTCACCCACC | 3018 |
| Hoxa11 | GCTACAAAGAAAGGAAGCCG | 3019 |
| Hoxa13 | GGGTCCCAGGACATTTCTCT | 3020 |
| Hoxa13 | GTAGTGGGTTCAAGGTGCCG | 3021 |
| Hoxa13 | GAATGCAACAGTGGATTGCC | 3022 |
| Hoxa13 | GATGCAGCAGCTATTCTCTC | 3023 |
| Hoxa13 | GGGCAAATCAATATTTACCC | 3024 |
| Hoxa13 | GCGGTGTTTACAGGCTGGAC | 3025 |
| Hoxa13 | GAACTGGTCAGACATCCAGA | 3026 |
| Hoxa13 | GCTAGACCCTCCCAAGGATG | 3027 |
| Hoxa13 | GCAGTAAGAAGGTAAACTCG | 3028 |
| Hoxa13 | GAAAGGACTCCCTGGGTGTG | 3029 |
| Hoxa2 | GAGGCAAGGAGGAAGCCAAA | 3030 |
| Hoxa2 | GTTTCATACCCGTAGGGCTC | 3031 |
| Hoxa2 | GTTCAAATGCTGATTATCTC | 3032 |
| Hoxa2 | GAAGGTGCTTTGCAGATGGA | 3033 |
| Hoxa2 | GATGGAAGGGTGGTGGCTTT | 3034 |
| Hoxa2 | GAAGCTGAGATGTGTTCTTA | 3035 |
| Hoxa2 | GACCTGCGTGTGGAGATTGG | 3036 |
| Hoxa2 | GGAGGGTAGACGACGACGTG | 3037 |
| Hoxa2 | GCTCCTAAACGCTGCTCTCT | 3038 |
| Hoxa2 | GTGGGTAGAGGCCATGATGA | 3039 |
| Hoxa3 | GTAGGAAAGACATGGAATTC | 3040 |
| Hoxa3 | GATAAGAATGGAGACCTTCG | 3041 |
| Hoxa3 | GGTATTGGCCGGGTGTGTGA | 3042 |
| Hoxa3 | GGACATGAAGGAGGCTTCTT | 3043 |
| Haxa3 | GCCAGAGAAAGAGGGATTCT | 3044 |
| Hoxa3 | GAAACTGGCCCAGCCTAGTC | 3045 |
| Hoxa3 | GGACGGGACATGAGGAGACA | 3046 |
| Hoxa3 | GCATCAAGGTCCAGCCTGGG | 3047 |
| Hoxa3 | GGCACTCCCAAACTACCTAT | 3048 |
| Hoxa3 | GCCATTAACCCTACTTCAGG | 3049 |
| Hoxa4 | GCAGTGCATGTGTATTTGTA | 3050 |
| Hoxa4 | GACCGATTGACAATTAGACC | 3051 |
| Hoxa4 | GAAGGCAAGAGATGCTTCTT | 3052 |
| Hoxa4 | GGCTGTGGAAGGTTCAGGAA | 3053 |
| Hoxa4 | GAGGTTCTATTAAGGAGGAT | 3054 |
| Hoxa4 | GCTCTGGAAAGGAGAGAGAA | 3055 |
| Hoxa4 | GTTCTGAAACGCGAAGTTAC | 3056 |
| Hoxa4 | GCTCGCTTCTCCCACCCTGA | 3057 |
| Hoxa4 | GCAGGGACTCCCTAACAGCC | 3058 |
| Hoxa5 | GGGTCCTGAAAGCTGCGAGG | 3059 |
| Hoxa5 | GGTGCCGTGTATGGGAGTCA | 3060 |
| Hoxa5 | GGCTGCTTGGAAGCTGGGAT | 3061 |
| Hoxa5 | GTCTGTGAAAGACGCTATCC | 3062 |
| Hoxa5 | GCAGTGCCCTGTTTGGTGCC | 3063 |
| Hoxa5 | GCGCGTTAGCGATCTCGATG | 3064 |
| Hoxa5 | GGCTGCTACTCTCCCACTGA | 3065 |
| Hoxa5 | GAGGACTGTGTTGGGCTGTC | 3066 |
| Hoxa5 | GCCAGGTGTGAGGTTCAGGC | 3067 |
| Hoxa5 | GGCACCTGTGGGCAGAAATG | 3068 |
| Hoxa6 | GAGCCTGGCTTGCAGGTGTG | 3069 |
| Hoxa6 | GCTTGTCAGGTTTCCTGTTT | 3070 |
| Hoxa6 | GTCCTGACAGAGTGGAGACC | 3071 |
| Hoxa6 | GCCGATGGTCAAGGTAATTC | 3072 |
| Hoxa6 | GGAGGGCGGTACTGAGAAGA | 3073 |
| Hoxa6 | GCGTCCCAAAGGCGTCCTGA | 3074 |
| Hoxa6 | GAGATTTGACTGGATGGAGG | 3075 |
| Hoxa6 | GATCCTTTGAGTGAAGCTCT | 3076 |
| Hoxa6 | GCCTGTACAAACAGTCTCCA | 3077 |
| Hoxa6 | GGAAGGCCCTGGCTTTGGTG | 3078 |
| Hoxa7 | GCTTAGAAAGGTGAAGCCGC | 3079 |
| Hoxa7 | GGGAACCACTTAGTCCTTTC | 3080 |
| Hoxa7 | GAGACCTGACAACCAGAGTT | 3081 |
| Hoxa7 | GGCTGTCTTGTGTAGATCTT | 3082 |
| Hoxa7 | GACCCTAAGGCGGCAATATC | 3083 |
| Hoxa7 | GAGTAAGAGAGAGAAAGAGA | 3084 |
| Hoxa7 | GCTGCTGAGATTGGCGGAGG | 3085 |
| Hoxa7 | GAGCCGCCAGGAGTGTATGA | 3086 |
| Hoxa7 | GCCAACAGATATACTAACAT | 3087 |
| Hoxa7 | GCAGTTTATGAGGCGTTTAG | 3088 |
| Hoxa9 | GGTGGAGAGCCTAATATTTG | 3089 |
| Hoxa9 | GTAGAGACCCAGCCAGAGAC | 3090 |
| Hoxa9 | GTTAGGGTGGTGTCTCTGTC | 3091 |
| Hoxa9 | GAAGGGTAAGCAACAAGGCC | 3092 |
| Hoxa9 | GATCAGGGAGGGCACAAACT | 3093 |
| Hoxa9 | GTCTCTGGCTGGGTCTCTAC | 3094 |
| Hoxa9 | GTCCTGCCTTGTGCAACTGA | 3095 |
| Hoxa9 | GGAGCCCTCTTCATCCACCA | 3096 |
| Hoxa9 | GTGTCGTGCTGTCGAGAGAA | 3097 |
| Hoxb1 | GAACCTATTGAAGGCCTTGG | 3098 |
| Hoxb1 | GTGATCTCTCCCAGGCCAAT | 3099 |
| Hoxb1 | GGTAACCCTTGAAACTTCTC | 3100 |
| Hoxb1 | GCCTGAGCTAGGGCAAGTCC | 3101 |
| Hoxb1 | GCGGAGGAAGCCAAAGCAGG | 3102 |
| Hoxb1 | GATGAGTTGATGGATAGGTA | 3103 |
| Hoxb1 | GATGCCGCATGGAAAGAGGA | 3104 |
| Hoxb1 | GAGAGGCTGAGGGAGAGAAA | 3105 |
| Hoxb1 | GGAGGGCAAGAGTTCAGGGA | 3106 |
| Hoxb1 | GCCTCAAATACATAAATCCA | 3107 |
| Hoxb13 | GGGAAATAGAGCCAATGTCT | 3108 |
| Hoxb13 | GTCCCAAGATTGCAGGAGCT | 3109 |
| Hoxb13 | GGTGAACAACAACCTGGATT | 3110 |
| Hoxb13 | GAAGGGCTGGGAGGCCACTT | 3111 |
| Hoxb13 | GGGAGCCAAGGCTGGITTCG | 3112 |
| Hoxb13 | GGGAGCAAAGCAGGAATCCT | 3113 |
| Hoxb13 | GCCAATCAGCGCTCATGCCC | 3114 |
| Hoxb13 | GGGTCTGGATTTCCGTTTAA | 3115 |
| Hoxb13 | GCTGCCTCAAAGGAGAACCC | 3116 |
| Hoxb13 | GGCTGCCTCAAAGGAGAACC | 3117 |
| Hoxb3 | GGCGACGCAGCTTTAAACAG | 3118 |
| Hoxb3 | GAACCGAGATTGGAGTCATA | 3119 |
| Hoxb3 | GTCCTGCGATGGTTTCGTTT | 3120 |
| Hoxb3 | GTCTTCTGGTTTCATTCTAA | 3121 |
| Hoxb3 | GGAACAGCGAGCACCGAAGG | 3122 |
| Hoxb3 | GAGGCAACGTAGCTGCATCC | 3123 |
| Hoxb3 | GCCAAGCATCCTAGAGGGTA | 3124 |
| Hoxb3 | GAAGCAGAGAGGCCTCCCTA | 3125 |
| Hoxb3 | GTTGCCTGTAGCCCTGGAGG | 3126 |
| Hoxb3 | GCTGCATCCTGGGCCATGAC | 3127 |
| Hoxb4 | GGGATAGAGAGATGCAAAGC | 3128 |
| Hoxb4 | GAACAAGGACCCAAGCTTCC | 3129 |
| Hoxb4 | GGAGAGGTGTCTGGGTGTGA | 3130 |
| Hoxb4 | GCTCCCACCTGCAGGCAACT | 3131 |
| Hoxb4 | GTCTTCTTGAAGGCAGTCAC | 3132 |
| Hoxb4 | GGCCTTGTGGGTTAAAGGGA | 3133 |
| Hoxb4 | GATCACAAACTAAAGGCTGT | 3134 |
| Hoxb4 | GCAGTTCATTTCCGAATGAA | 3135 |
| Hoxb4 | GACAGAGGCGGCGGCTTTAG | 3136 |
| Hoxb4 | GAGCTCCAAGGGAGAGGAAT | 3137 |
| Hoxb5 | GAGAGACACAACCAACGCTG | 3138 |
| Hoxb5 | GCCAGAATCTATCATCGAGT | 3139 |
| Hoxb5 | GCATCTGGCGAGCTTGTTAA | 3140 |
| Hoxb5 | GTCTTTCAGGTCCCTGCTGA | 3141 |
| Hoxb5 | GCGAAGGGAGAGGTCTGTGG | 3142 |
| Hoxb5 | GACGCGAAGGGAGAGGTCTG | 3143 |
| Hoxb5 | GGTAGTGTCTCACAGCTCCC | 3144 |
| Hoxb5 | GTTGCACAGAGCCAGCAAAG | 3145 |
| Hoxb5 | GTCTCAGCTCAGTGCGGAGG | 3146 |
| Hoxb5 | GAGTCCAGGAGGGAATCTGG | 3147 |
| Hoxb6 | GAGCAAGCATGCCAGTTTGA | 3148 |
| Hoxb6 | GAAGCTGTCTTTGTGAACTG | 3149 |
| Hoxb6 | GGGTTGCAGCGGTCAGTTCT | 3150 |
| Hoxb6 | GAGGCCAGGCCAGCAAGTAG | 3151 |
| Hoxb6 | GCTGCAAACCGCACAGGTGG | 3152 |
| Hoxb6 | GTTGGATACACTGTTTGTCT | 3153 |
| Hoxb6 | GAACCACCTCGGAGCTCTTA | 3154 |
| Hoxb6 | GCACACACACACACAGGAGG | 3155 |
| Hoxb6 | GGATTTATTTGGCTGCAATG | 3156 |
| Hoxb7 | GTAGTAACTAGATGTGACCA | 3157 |
| Hoxb7 | GGAAGGGAGGAAGGAGGCTT | 3158 |
| Hoxb7 | GCAACTTGGTGGGTGGGTGC | 3159 |
| Hoxb7 | GAGTCAGATAGGGATTAAAT | 3160 |
| Hoxb7 | GGAGAAAGAGAAGCTGGAGC | 3161 |
| Hoxb7 | GGGAAGAGATCTACCCAGGC | 3162 |
| Hoxb7 | GCCGTCATACCATTGGCCGA | 3163 |
| Hoxb7 | GAACTCCTTCTCCAGCTCCA | 3164 |
| Hoxb7 | GGAGGAGAGAGGATCGAGGG | 3165 |
| Hoxb7 | GAGGAGAGAGGATCGAGGGA | 3166 |
| Hoxb8 | GGAAGCCGCAGCTCTCACCT | 3167 |
| Hoxh8 | GGAATAAAGTGCAGGACAAT | 3168 |
| Hoxb8 | GACAATGGGTCAGGTGAGAC | 3169 |
| Hoxb8 | GGAAAGAGAAGAAGCCACAC | 3170 |
| Hoxb8 | GGAAGCCAGTCCTTCTGGGA | 3171 |
| Hoxb8 | GAAATAATAGGCACAAATCA | 3172 |
| Hoxb8 | GATTCTCTCTTCAGCAGGTG | 3173 |
| Hpxh8 | GTCATGATTTGAGGACTCAC | 3174 |
| Hoxb8 | GCTGAAATGAGACCGATTAT | 3175 |
| Hoxb8 | GCATAAcACAGCAGTAACCA | 3176 |
| Hoxb9 | GACTGTGTGTGTGCTCTCGG | 3177 |
| Hoxb9 | GGAGGCTAAGGAGGGAGTCA | 3178 |
| Hoxb9 | GGCCCTGGAACTAGAGTTTC | 3179 |
| Hoxb9 | GCAGCTGAGAGAGGCGAAAG | 3180 |
| Hoxb9 | GGATGGAAAGGAAGGTAACC | 3181 |
| Hoxb9 | GGAGCGAATGAATCATAGTT | 3182 |
| Hoxb9 | GCAAAGCCCGGGAGAGGAAT | 3183 |
| Hoxb9 | GCCCGACAGGGTAATTAAAG | 3184 |
| Haxb9 | GGGAGGACCAGCATACAGGG | 3185 |
| Hoxb9 | GTTAAGTATCTGTAGGTCCT | 3186 |
| Hoxc10 | GCCAGGCAGGGACAATAGGA | 3187 |
| Hoxc10 | GACTGTCCCAAGTCTGGTCT | 3188 |
| Hoxc10 | GAGAGCGCTTGTGTGGGTCC | 3189 |
| Hoxc10 | GCAGGAAGCATTTCTCCTGA | 3190 |
| Hoxc10 | GGACTGTCCCAAGTCTGGTC | 3191 |
| Hoxcl0 | GAAAGTGTAAGGTGAAGAGA | 3192 |
| Hoxc10 | GGTCTAGCCGTCACATGGTG | 3193 |
| Hoxc10 | GTGTTATTCAGGGCAAGGTT | 3194 |
| Hoxc10 | GTGGAGTGT6TGGCCAGCAG | 3195 |
| Hoxc10 | GAGTCTCCAGTGTCTGGAGT | 3196 |
| Hoxc11 | GTAATAGCCAAAGGGACTGG | 3197 |
| Hoxc11 | GGAAGTCTCTTCTACAATAT | 3198 |
| Hoxc11 | GCACAGCCTTGGAGAGAGGT | 3199 |
| Haxc11 | GCTAGACAAAGTTGGGACAC | 3200 |
| Hoxc11 | GCAAGGAGGGTTTATAGACT | 3201 |
| Hoxc11 | GGAGGAGAGAGAGAGAGGGT | 3202 |
| Hoxc11 | GACTTGGAGAAGGGCAGGGT | 3203 |
| Hoxc11 | GAGCACTTCGCAGACGTAGG | 3204 |
| Hoxc11 | GCAACAGAATCTTCTGTTTC | 3205 |
| Hoxc11 | GCTCTGATTCTTCAGGTAGA | 3206 |
| Hoxc12 | GAACATCTGCAAGTCAACAT | 3207 |
| Hoxc12 | GGCTAAGGGAGGGAACCAGG | 3208 |
| Hoxc12 | GGTGATAAGATAATACATCT | 3209 |
| Hoxc12 | GGAGATTAGCATTGTCGGAA | 3210 |
| Hoxc12 | GCGTCCIGTAGAGGAGAGAG | 3211 |
| Hoxc12 | GTGTTGCACAGAAGGAAGAG | 3212 |
| Hoxc12 | GAGAAATCCACGTCTGAAGA | 3213 |
| Hoxc12 | GGTTGCAGAGAGAATGAGAA | 3214 |
| Hoxc12 | GAAGGAGAACCGGCCAAGCG | 3215 |
| Hoxc12 | GAGATTACCCTACAACCTGC | 3216 |
| Hoxc13 | GACTACCGAAGTCTCTAAAT | 3217 |
| Hoxc13 | GTAATTACATCTCATTTCGG | 3218 |
| Hoxc13 | GTAGCAGGCACGGAAGGTCT | 3219 |
| Hoxc13 | GCTGCTGGAGTCCAAGGTCA | 3220 |
| Hoxc13 | GGTCTAGGATTAGTCTTGAT | 3221 |
| Hoxc13 | GCGTAGTGGGAATGCGGCTA | 3222 |
| Hoxc13 | GGCTCCGGTTCTCAAACAGA | 3223 |
| Hoxc13 | GTGATAAGCGCTAAGGAGCC | 3224 |
| Hoxc13 | GCTGTGGTCACGTGGGAACC | 3225 |
| Hoxc13 | GGGAGCTTGGCACAATTCCA | 3226 |
| Hoxc4 | GACTTGAGGATCCGTGAATG | 3227 |
| Hoxc4 | GAACTACAAGTTGCTGGAAG | 3228 |
| Hoxc4 | GGGAAGGACAGTGGGTAGCA | 3229 |
| Hoxc4 | GACAGGGTCCCAGCAGTACT | 3230 |
| Hoxc4 | GGGCTTCAGTGCAGGTTGGA | 3231 |
| Hoxc4 | GATGTCATTTCTGGAAGTCT | 3232 |
| Hoxc4 | GTGTGTGGGTGACAGAGGGA | 3233 |
| Hoxc4 | GGGAAAGCAGCCAGAGGCAC | 3234 |
| Hoxc4 | GCCCACACAGGCTTCCCTTG | 3235 |
| Hoxc5 | GCAGGAAGAAAGGCCCGCGT | 3236 |
| Hoxc5 | GATGACTGAGAAAGAGAGTT | 3237 |
| Hoxc5 | GAAGCTTGAGTGAGCCGGGT | 3238 |
| Hoxc5 | GGGAGGTTAGTGATGGAAGC | 3239 |
| Hoxc5 | GATGAGCAAGGGAGAAGAGA | 3240 |
| Hoxc5 | GCCTTCTAGCAGTCAGTTTG | 3241 |
| Hoxc5 | GGTCTCCTAGGCCTAGGCGA | 3242 |
| Hoxc5 | GAAGTCTACCCAAGTTCACC | 3243 |
| Hoxc5 | GAGACCTTGACCTTTAGTTT | 3244 |
| Hoxc5 | GCTCAGAAGCCGAAGATCCC | 3245 |
| Hoxc6 | GCTGTTGGAAACCTCTGCCC | 3246 |
| Hoxc6 | GCATCCCGAAAGAGGAAATT | 3247 |
| Hoxc6 | GAAATGGACTTTCTCCCTTT | 3248 |
| Hoxc6 | GTTTCCCTGGAGTGTCACTA | 3249 |
| Hoxc6 | GGACCCTCTTTCTACTGGGA | 3250 |
| Hoxc6 | GCCTTACAACTCAGGTCCAG | 3251 |
| Hoxc6 | GCAAGCCAGATGTCAAGAAA | 3252 |
| Hoxc6 | GAAACATGGTGCACAGAGGA | 3253 |
| Hoxc8 | GCTCTTTCCTCTAACAGCCC | 3254 |
| Hoxc8 | GTCATCAAAGAAAGAATGGC | 3255 |
| Hoxc8 | GGGTACATGATCACCATGCT | 3256 |
| Hoxc8 | GCTGACATTTCTGGCCAGAG | 3257 |
| Hoxc8 | GTGTGCTTCTAAGCCCAGGC | 3258 |
| Hoxc8 | GTTTGCAGGTTAGGCAAGGA | 3259 |
| Hoxc8 | GAGATGGGTCCTCACTCTAC | 3260 |
| Hoxc8 | GGTGGCCTCACATACTGTAG | 3261 |
| Hoxc8 | GAGGGTCCAGATCCTCTCTG | 3262 |
| Hoxc8 | GCTCAGTACAGAACTGAACA | 3263 |
| Hoxc9 | GTGACGTGAAGGCGGCAAAC | 3264 |
| Hoxc9 | GGCGAGACATCTCAGAGATC | 3265 |
| Hoxc9 | GCTTTGTGTGGGTCCTTGCT | 3266 |
| Hoxc9 | GAAGTGGAGCAAGGTCTCAT | 3267 |
| Hoxc9 | GTCTCAGACAGACAGGCAAG | 3268 |
| Hoxc9 | GTCCAGAGCAGGTTGTCCGC | 3269 |
| Hoxc9 | GGATTCTCTGAAACTCGGCA | 3270 |
| Hoxc9 | GATGATGGATTTAAAGGAGG | 3271 |
| Hoxc9 | GCACACAGCCAGTTTGGGTA | 3272 |
| Hoxc9 | GGTGAAGGAAGATATGTATA | 3273 |
| Hoxd1 | GAGCCCTGGACATCAGCTCC | 3274 |
| Hoxd1 | GAATGAAATGACCAGAGGTT | 3275 |
| Hoxd1 | GCTCCTGGGACAGGTATTGC | 3276 |
| Hoxd1 | GTTTATAATCATCTGAGGAG | 3277 |
| Hoxd1 | GGGCTCACTCCTGGACTATG | 3278 |
| Hoxd1 | GGCTAAGTTGGCAGCAAGGC | 3279 |
| Hoxd1 | GTGTGCTTAAGTATCTCCCA | 3280 |
| Hoxd1 | GTCCACCTCACTAGCATAAT | 3281 |
| Hoxd1 | GACCTTCTCAGAGGGAGGGC | 3282 |
| Hoxd1 | GGTATTGCGGGAGAAAGGCA | 3283 |
| Hoxd10 | GAAGGACGGCTCCCACACAC | 3284 |
| Hoxd10 | GTGGAGGCTCTGGGCCTAAG | 3285 |
| Hoxd10 | GGCCGGGAGAAATTCCTTTA | 3286 |
| Hoxd10 | GGAGAGACGCTTTCGCGAAT | 3287 |
| Hoxd10 | GGTGCTAATCAGTGGTTGTT | 3288 |
| Hoxd10 | GTCTTCCGTTTCCTCTGGTG | 3289 |
| Hoxd10 | GTCTCTGGGCCTGAAATCCA | 3290 |
| Hoxd10 | GGGCAAGAAGGGAATAAAGA | 3291 |
| Hoxd10 | GTGGTTGTTCTTTAATGAGC | 3292 |
| Hoxd10 | GGGCTGGTTAATTTAGTACT | 3293 |
| Hoxd11 | GAAGGGAGTGGTACTAAGCC | 3294 |
| Hoxd11 | GAGATTGCTCAGGGCTTAGT | 3295 |
| Hoxd11 | GATTTCTGTGGATCAGTAAA | 3296 |
| Hoxd11 | GCCATGTCGTTGAACTTGAA | 3297 |
| Hoxd11 | GAGAACCAACCGATCTCCCT | 3298 |
| Hoxd11 | GATGTTGTGCATCTTGCTAA | 3299 |
| Hoxd11 | GATAGGTGAGGCTGGAGCAG | 3300 |
| Hoxd11 | GCCAGCAGACTTCACTTTAG | 3301 |
| Hoxd11 | GGACTAGGTGTGAGAGTGTG | 3302 |
| Hoxd11 | GAAGCATTTCTCTCTCTACG | 3303 |
| Hoxd12 | GTTCAACTAACTTGCACATC | 3304 |
| Hoxd12 | GAACAGCGTGAAGATTCCTT | 3305 |
| Hoxd12 | GAGGGAAGGTGGGAGGAGGA | 3306 |
| Hoxd12 | GGAATGAAGTGGGTCGATTA | 3307 |
| Hoxd12 | GGGTCAGTTGCTACAACCTC | 3308 |
| Hoxd12 | GCAGCCTGCGAAATAAGGGC | 3309 |
| Hoxd12 | GAGCCAAAGCCTGTTGAGGG | 3310 |
| Hoxd12 | GGTCCTGCTTTAGGCTAGCG | 3311 |
| Hoxdl2 | GTGATGTGCTTCCCTTTCCA | 3312 |
| Hoxd13 | GTGAGCTCTGATTTGAATCT | 3313 |
| Hoxd13 | GTGGCTGCAAAGTCAACTCC | 3314 |
| Hoxd13 | GGATGAGCTGTCTCGAATTT | 3315 |
| Hoxd13 | GGGTGCGTGAGCCTCAAAGT | 3316 |
| Hoxd13 | GGTTAGTCAAGAGTGCTGGG | 3317 |
| Hoxd13 | GCTCTGATTTGAATCTAGGT | 3318 |
| Hoxd13 | GACTGCTGAGGCTGATTATG | 3319 |
| Hoxd13 | GGATTTGGAGTTCTACCTGT | 3320 |
| Hoxd13 | GTGTAGGTTATGAGAGGTAC | 3321 |
| Hoxd13 | GATTGCGCGACGGCCCATCT | 3322 |
| Hoxd3 | GGGCTGCTTAGTTCTGGGTC | 3323 |
| Hoxd3 | GAATTTACTGCAATTCCTTG | 3324 |
| Hoxd3 | GTCCTCTTTGGAGATACCGC | 3325 |
| Hoxd3 | GTTTCCAAATAAAGACCTTG | 3326 |
| Hoxd3 | GAAGTCCGATGGGTTGAGTG | 3327 |
| Hoxd3 | GGTTATCAGGATGCTCAAAC | 3328 |
| Hoxd3 | GGTTAGAGGGACAGGAAAGG | 3329 |
| Hoxd3 | GCCTTTCTGGAACAGGGCTA | 3330 |
| Hoxd3 | GGCTCTGGGAAAGCAGAATG | 3331 |
| Hoxd3 | GTGTCCATGGGRGAAAGGGC | 3332 |
| Hoxd4 | GGCGGTGATGGTACTCACAG | 3333 |
| Hoxd4 | GAGGCAATACCCAGTTTACT | 3334 |
| Hoxd4 | GATTGAAATAAAGGCGGTGA | 3335 |
| Hoxd4 | GGCTCTAGCTAAATGAGAAG | 3336 |
| Hoxd4 | GGATTTATGCTTAAGTACAC | 3337 |
| Hoxd4 | GTTCCTACTGGGAGTTGCAA | 3338 |
| Hoxd4 | GGAGTTGCAATGGCAGCGGA | 3339 |
| Hoxd4 | GCCTTCCTGCAGCATCTGTA | 3340 |
| Hoxd4 | GTGTACTTAAGCATAAATCC | 3341 |
| Hoxd4 | GAAGTGGGTTTGCAAATGGC | 3342 |
| Hoxd8 | GGGTGGCATTTCCTAGGGCA | 3343 |
| Hoxd8 | GTCCTCAAGATCAGAGAGCC | 3344 |
| Hoxd8 | GGGAAGAAGGAATCATGCCG | 3345 |
| Hoxd8 | GTGCTGAACCCTTTATCCTT | 3346 |
| Hoxd8 | GAATAGGTCTGGGAATGGAA | 3347 |
| Hoxd8 | GCCTCCGCCAGCCTTAAAGG | 3348 |
| Hoxd8 | GGAGCCGGAGAGGAAACTGG | 3349 |
| Hoxd8 | GTTTCCTCTCCGGCTCCAGG | 3350 |
| Hoxd8 | GCAGATTTGCACAGGTGCCA | 3351 |
| Hoxd8 | GGAGGCGAGAGAATGTGGGA | 3352 |
| Hoxd9 | GTTCCTTCTGCTTTCTGAAA | 3353 |
| Hoxd9 | GGGAAAGAGAAAGGGACTTG | 3354 |
| Hoxd9 | GGAGATCGCAGGACCCAGAG | 3355 |
| Hoxd9 | GTTATGAAGACTGAGCTCTC | 3356 |
| Hoxd9 | GTTGCTTGTTCCTGCAATGG | 3357 |
| Hoxd9 | GGAACCGCATCTCCGAGGGT | 3358 |
| Hoxd9 | GGAGCCGACAGTGATGGCCA | 3359 |
| Hoxd9 | GTTCTTACTTACCAGGCTCA | 3360 |
| Hoxd9 | GCAGTGGGTTCTTACTTACC | 3361 |
| Hoxd9 | GGTTCCTGCAGGCCTCTCTG | 3362 |
| Hsbp1 | GAAGGCGGAGCTAAGAACTG | 3363 |
| Hsbp1 | GTTTAGTAAAGGAGAGAAAC | 3364 |
| Hsbp1 | GAATTGTACAGGCACATGGA | 3365 |
| Hsbp1 | GGAACATGGAGAACTCTGGC | 3366 |
| Hsbp1 | GCGCCGAGAAACCGAAGTGT | 3367 |
| Hsbp1 | GTGTCCTAGGAAGGAAAGAG | 3368 |
| Hsbp1 | GTAATTCCCATCTCTGTGTT | 3369 |
| Hsbp2 | GGCGTAGCTTATCCGCAGGC | 3370 |
| Hsf1 | GCTTTCCGACTTGTCCGTCA | 3371 |
| Hsf1 | GGAGCTCAAATGTGTGACGA | 3372 |
| Hsf1 | GTGTCAGTTTCAGGACCCTT | 3373 |
| Hsf1 | GAAAGAGGAAGTGCCTGCCT | 3374 |
| Hsf1 | GCAGCGATGCCGGTGACATG | 3375 |
| Hsf1 | GGGTGAGGGACCAGCTTCCA | 3376 |
| Hsf1 | GGCTCTCCTGCACATGAGGA | 3377 |
| Hsf1 | GTCCACAGAGGCAGGAGAGG | 3378 |
| Hsf1 | GGTCTTGCCAAGTGCCTGCA | 3379 |
| Hsfl | GCTCTGATTGGTGAGCAGCC | 3380 |
| Hsf2 | GTGCATCCGAAGCCTTGGAT | 3381 |
| Hsf2 | GCAGAGTGCAGAGAAGCCCA | 3382 |
| Hsf2 | GCGAGTCCAATCCAAGGCTT | 3383 |
| Hsf2 | GGATTGGACTCGCTGAGACC | 3384 |
| Hsf2 | GCACTACAGAGCAAAGCGAT | 3385 |
| Hsf2 | GGATTCGCATGGAAAGGGTT | 3386 |
| Hsf2 | GTCTCAGCGAGTCCAATCCA | 3387 |
| Hsf2 | GATTCGCATGGAAAGGGTTT | 3388 |
| Hsf4 | GAATTTATGGTGCCTGAGAT | 3389 |
| Hsf4 | GCAGGTCGAGGTGGCGAAGT | 3390 |
| Hsf4 | GACTCGAGATCACAGGACGC | 3391 |
| Hsf4 | GGAAATACACAAAGGCTGGA | 3392 |
| Hsf4 | GACATTCAAGGAGACCTCAA | 3393 |
| Hsf4 | GCCTTTGTACTGTGACTGTG | 3394 |
| Hsf4 | GACACATTGAAGCCTAGGTA | 3395 |
| Hsf4 | GGCCTTTGTACTGTGACTGT | 3396 |
| Hsf4 | GGGCCTTTGTACTGTGACTG | 3397 |
| Hsp90ab1 | GAAGGTCTCTGTGAATAATG | 3398 |
| Hsp90ab1 | GCTGACCTGGATCGGTCACA | 3399 |
| Hsp90ab1 | GGTCGTTCAACCCTGGCCCA | 3400 |
| Hsp90ab1 | GATCTGCTACCATGACGTCA | 3401 |
| Hsp90ab1 | GCAGGCCACCTTTAGAACAG | 3402 |
| Hsp90ab1 | GGACTGAAAGAGAATGGAGG | 3403 |
| Hsp90ab1 | GGAGCATGCATATGCAATTA | 3404 |
| Hsp90ab1 | GAGAGGCTAACAGACAGTGC | 3405 |
| Hsp90ab1 | GGCCTCCTTAAAGTTGGACA | 3406 |
| Hsp90ab1 | GTACAGCACAGCTTCAGGCT | 3407 |
| Id1 | GAGTGTGAGGAGCTGAGGAG | 3408 |
| Id1 | GACGCTGACACAGACCAGCC | 3409 |
| Id1 | GAACGTTCTGAACCCGCCCT | 3410 |
| Id1 | GGTCTCTTTCTCACTTCTCC | 3411 |
| Id1 | GCGAAGGAATCCAACTCAGC | 3412 |
| Id1 | GGCTCAAGAACTGAAAGGGT | 3413 |
| Id1 | GCATAGGTAGAGCAGCTAGT | 3414 |
| Id1 | GATCCAGAGGTGGGACCCAG | 3415 |
| Id1 | GGCTCAGACCGTTAGACGCC | 3416 |
| Id1 | GACCAGCCCGGGAAAGGAAA | 3417 |
| Id2 | GACGTGCCCAGCTGCAGTAA | 3418 |
| Id2 | GTCTTAAGTTTCGAGTGATT | 3419 |
| Id2 | GTAACCCTGCCTCATTCTTG | 3420 |
| Id2 | GGGTGGTGGGAAGGTGTGAA | 3421 |
| Id2 | GGGCATCCCTGAATTGCCAT | 3422 |
| Id2 | GCAGCCAATGCCTGTAGGGT | 3423 |
| Id2 | GCCTCCTTAGAGAGAAGCCC | 3424 |
| Id2 | GATCCCGCCCTTACTGCAGC | 3425 |
| Id2 | GCCTCATTACCCCAACAGAA | 3426 |
| Id2 | GCTCATTATCATCCAGCCCA | 3427 |
| Id3 | GCTGATACCGAGGAGAGGCG | 3428 |
| Id3 | GCTGATACCGAGGAGAGGCG | 3429 |
| Id3 | GCTTTCTTAATCAATCAGCC | 3430 |
| Id3 | GCTTACCTGTGATGTGATAC | 3431 |
| Id3 | GGCGTGGAAAGGACTGAATG | 3432 |
| Id3 | GTGCAAAGTGTGCAAAGGGA | 4433 |
| Id3 | GTTCTATGTATGCCCGTGGA | 3434 |
| Id3 | GCAGGCAAGAGAGAGTCTTT | 3435 |
| Id3 | GAACGCATGACGTCCCACCC | 3436 |
| Id3 | GAGTGTGCAAAGTGTGCAAA | 3437 |
| Id4 | GTTACATCCATCTATGAAAC | 3438 |
| Id4 | GGGAATGACGCTCGGGCCAA | 3439 |
| Id4 | GGGACTCTGAGCCTTGTTTG | 3440 |
| Id4 | GGTGGCACTGTCCTCCTGAT | 3441 |
| Id4 | GAGCTTAAAGGTAGCAGTAT | 3442 |
| Id4 | GCCTTCTCTATAGACAGCGT | 3443 |
| Id4 | GAGGAGCATGAAGCCCATCC | 3444 |
| Id4 | GCCTTCTGACCCTCCAAAGG | 3445 |
| Id4 | GCCTCTAAGGATTTAGAGGG | 3446 |
| Id4 | GACGCTCGGGCCAATGGGAA | 3447 |
| Ikzf1 | GGAAGGCTCTGGGCCTCAAA | 3448 |
| Ikzf1 | GTGCACACGCCAGCGTGGAA | 3449 |
| Ikzf1 | GCTAGACTGGTGGTAAGGAA | 3450 |
| Ikzf1 | GCTCAGGGTTAACGCCTGTC | 3451 |
| Ikzf1 | GAGGAGTGAGCCACAGGGAC | 3452 |
| Ikzf1 | GCGCCAACCCAAAGTTTGCA | 3453 |
| Ikzf1 | GGCTGCAAAGTTTGTGTGCG | 3454 |
| Ikzf1 | GGGCTTTCTGATATCATCTT | 3455 |
| Ikzf1 | GCTGGTGGAAAGGAAGACAC | 3456 |
| Ikzf2 | GTTGGCCTAGGTTCCTTGCT | 3457 |
| Ikzf2 | GGCAGTGGATCTGTAGCTAA | 3458 |
| Ikzf2 | GGTTATCCAATCTTTCTTCT | 3459 |
| Ikzf2 | GGGAAGTTCTCTCTCTGGCC | 3460 |
| Ikzf2 | GCTCCGCCGGATCGGTTTCT | 3461 |
| Ikzf2 | GCCACATCCACCCGAGTCAA | 3462 |
| Ikzf2 | GTCGATTCTTAAGGAACCGG | 3463 |
| Ikzf2 | GCAGTGCACAAACACACGTT | 3464 |
| Ikzf2 | GGTTTCTCAGAAATGTTGTT | 3465 |
| Ikzf2 | GAGGATCTGGGACACTGAGC | 3466 |
| Ikzf3 | GTCAGATGACAAGGTTTGTC | 3467 |
| Ikzf3 | GCACTAGTTAATGTGAGCTC | 3468 |
| Ikzf3 | GCATTTCTAACGATGCCAAC | 3469 |
| Ikzf3 | GAGGAACTGTACACTTTCAC | 3470 |
| Ikzf3 | GGGTAGAGGGACTAAGTCAA | 3471 |
| Ikzf3 | GTTCCAGGTCCTTCCGTGTC | 3472 |
| Ikzf3 | GAAAGCATTTGACAGGAGGG | 3473 |
| Ikzf3 | GACGTCTACTTGAGAAACAC | 3474 |
| Il6 | GAGAAAGCCTCTTCCAGATG | 3475 |
| Il6 | GAAAGCACACGGCAGGGAAT | 3476 |
| Il6 | GCAGAGAATAGGCTTGGACT | 3477 |
| Il6 | GAACTCTTGTCAGCCTCATC | 3478 |
| Il6 | GAAGCCCTGGTCTTCACAAA | 3479 |
| Il6 | GAGAATGCAGAGAATAGGCT | 3480 |
| Il6 | GTGAAGACCAGGGCTTCACA | 3481 |
| Il6 | GTAACCCAGTGTAAACACAC | 3482 |
| Il6 | GGTCATGCAAGGAGGCTTGA | 3483 |
| Il6 | GTCTGTAGCTCATTCTGCTC | 3484 |
| Ilf2 | GTGAATGGTAAGCTCTTCTA | 3485 |
| Ilf2 | GGAGTAGATCCTAAGGACCC | 3486 |
| Ilf2 | GGCTTCTTTCACTTGTCCCA | 3487 |
| Ilf2 | GACTCACCTGGACTTGGCTG | 3488 |
| Ilf2 | GAACAGGTAGAAGACTCACC | 3489 |
| Ilf2 | GAGGGAATAGTGGTGGTAAG | 3490 |
| Ilf2 | GCCAATAAGAAGACATAACA | 3491 |
| Ilf2 | GTGAGTCTTCTACCTGTTCC | 3492 |
| Ilf3 | GGTGGATCGCCCACGTGATG | 3493 |
| Ilf3 | GGTCCGGAGCTCTTCATATC | 3494 |
| Ilf3 | GGGAACACCGGCAAGGTAAG | 3495 |
| Ilf3 | GCACATGCGGTTGTCAACAC | 3496 |
| Ilf3 | GCCAAGAGGACGCTAGTGAC | 3497 |
| Ilf3 | GTGGATCGCCCACGTGATGC | 3498 |
| Ilf3 | GATGGAAGAGCCGAGCGAAG | 3499 |
| Ilf3 | GATATGAAGAGCTCCGGACC | 3500 |
| Ilf3 | GACCTGAGGTGCTTCTGATC | 3501 |
| Insm1 | GCCTGTGGAGTTGACCCAAG | 3502 |
| Insm1 | GGCTCCCTCTGAGGACAGAT | 3503 |
| Insm1 | GGTGTCCACTTGGGACACTT | 3504 |
| Insm1 | GGACCGCAGCTGCATCCATA | 3505 |
| Insm1 | GGGTGAGCTTTGCTCTTCTC | 3506 |
| Insm1 | GAAGGAGGAGACCCACAGGT | 3507 |
| Insm1 | GACAGGGACGCGTCCATGAA | 3508 |
| Insm1 | GTACATCTGCCGCACCTACC | 3509 |
| Insm1 | GAGCAGCAGACCGTGAAGGG | 3510 |
| Irf1 | GTTCTAGCTAGCGGTGACCA | 3511 |
| Irf1 | GAGCGATTCGCAGAGGGTGC | 3512 |
| Irf1 | GACGAAGGAGTGGTGCGCAC | 3513 |
| Irf1 | GCTGGGAGTCTGCAGAAAGA | 3514 |
| Irf1 | GCTTTCAGTAGGGTCTCTGT | 3515 |
| Irf1 | GCACGGGACACCAGGAAGTG | 3516 |
| Irf1 | GCGAAAGATGCCCGAGATGC | 3517 |
| Irf1 | GAGACACTCTGACCAGCCAA | 3518 |
| Irf2 | GCAGGTGTAACTAAATGTAA | 3519 |
| Irf2 | GAACTTCCGCACCTCCAGGC | 3520 |
| Irf2 | GTTCTGAGCACTTAAGCCAC | 3521 |
| Irf2 | GTCTTCTCTTCCCTAGAACA | 3522 |
| Irf2 | GATTCCACAGACAGGGATAA | 3523 |
| Irf2 | GTGGTGGCCGTAGGGAAGGA | 3524 |
| Irf2 | GCCTTTCCTCTCCCTGTTCT | 3525 |
| Irf2 | GCTAGACCGTGTGGGAAAGA | 3526 |
| Irf2 | GCAGGAACGTTGTTAGTTCC | 3527 |
| Irf3 | GAACTCACCTGGGTGGAGTT | 3528 |
| Irf3 | GGTGCTTGGAAGTCACAGCT | 3529 |
| Irf3 | GGTGTGACAAAGACTTGAAA | 3530 |
| Irf3 | GGTCACTTGTGAAACTTTCA | 3531 |
| Irf3 | GTCAGATTACCAACTGGCCA | 3532 |
| Irf3 | GAAGAGGGCGCCTAACTCCA | 3533 |
| Irf3 | GATCTTCCATGAAAGGATGA | 3534 |
| Irf3 | GTTCCCAGCATGCCTGTAGG | 3535 |
| Irf3 | GAGCAATTCCGTGGTTGACC | 3536 |
| Irf3 | GAGACCCAACTCTTCAGAGC | 3537 |
| Irf4 | GTGGTTGTCAGGGCTCACAG | 3538 |
| Irf4 | GAAGTGATAGTTTAACAGTG | 3539 |
| Irf4 | GTTGCCATGATTGAAACTTT | 3540 |
| Irf4 | GAAACAAGGTCTCCGTCTCT | 3541 |
| Irf4 | GGCAGACTGGTTAAAGACAT | 3542 |
| Irf4 | GAACTTTATAGACCGGGAGG | 3543 |
| Irf4 | GTTCTTAGTGGTCAGCTAGA | 3544 |
| Irf4 | GGAGGAGCTGAAGAAAGCCA | 3545 |
| Irf4 | GCTTCGGACTAGAGCCCACC | 3546 |
| Irf4 | GGTATGCTGTTTGCAAGGAA | 3547 |
| Irf5 | GCAATTGTGAACTGGCAGGC | 3548 |
| Irf5 | GGCAGGAGGGAGCTTCTGTG | 3549 |
| Irf5 | GATCTCTGAGTTGTCCCATC | 3550 |
| Irf5 | GCTTTGCAGGTTCTCTGGAC | 3551 |
| Irf5 | GAAGGCCCGTTTATGGAACC | 3552 |
| Irf5 | GAGCTGTGTGCCGACAGGGT | 3553 |
| Irf5 | GTCGATGGAGCCACACTCCA | 3554 |
| Irf5 | GTTGCCTTGAACTGGGTGTG | 3555 |
| Irf5 | GCTAGCTAAAGTGAACAATG | 3556 |
| Irf5 | GGACCAGAAAGGATGTGGAC | 3557 |
| Irf6 | GTGACATCCCAAACTGAGCT | 3558 |
| Irf6 | GTGCAGGGTCACTACGGGAG | 3559 |
| Irf6 | GGACATTTGCTTGGTTTCAA | 3560 |
| Irf6 | GGGAAAGCTCAGGTCTTCCC | 3561 |
| Irf6 | GATGTCACCGGGCAAAGGCT | 3562 |
| Irf6 | GTGGCACTTGTCAGGCACAC | 3563 |
| Irf6 | GTGTTGTGATCGACTGAGGG | 3564 |
| Irf6 | GTCCACCACTCAGGAGACTG | 3565 |
| Irf6 | GAAGGGTTTGCCTCACTGCC | 3566 |
| Irf7 | GGCTGCTTTGGCAATGAACA | 3567 |
| Irf7 | GAATTCCAGAGTCTTAAGGC | 3568 |
| Irf7 | GCTTTCCTCTTAGCTACAGT | 3569 |
| Irf7 | GAACGTGCGTGTGGAGTGGA | 3570 |
| Irf7 | GACAGCTTCACGTGAGGGAG | 3571 |
| Irf7 | GTGGGTAGACCTTTAGGGAA | 3572 |
| Irf7 | GCCAGTGCCTCGGGAAGTGA | 3573 |
| Irf7 | GCATGCCATGACTGCTGTTC | 3574 |
| Irf7 | GTGTGTAACTACCGTAGCCC | 3575 |
| Irf7 | GGTGTTTGGGACCCTCATGA | 3576 |
| Irf8 | GAGCACCGATTCTCCTCAGA | 3577 |
| Irf8 | GCGCGAGCTAATTGAGGAGC | 3578 |
| Irf8 | GGGAGAGGTGTTTGTTCATT | 3579 |
| Irf8 | GCAGGAAATCTGGGAAACCA | 3580 |
| Irf8 | GCATGTGCAGGGCTTAACTA | 3581 |
| Irf8 | GGAAACCCTGACCTCAGCAG | 3582 |
| Irf8 | GCCTGAGCAGCTGACACTCA | 3583 |
| Irf8 | GGCCTCTAAGGATGAGGGTG | 3584 |
| Irf8 | GTGGCCCAGGGCTGAATGAA | 3585 |
| Irf9 | GAAGGACCACCAAGAAGCCT | 3586 |
| Irf9 | GTGAACATATGCAAGATGGA | 3587 |
| Irf9 | GCAGTAAGCTGAGGTCTCTG | 3588 |
| Irf9 | GGCAGTAAGCTGAGGTCTCT | 3589 |
| Irf9 | GGCAACACGGCTTAGTCATT | 3590 |
| Irf9 | GGAGAATTGAAACTTAGGGT | 3591 |
| Irf9 | GAGAATTGAAACTTAGGGTG | 3592 |
| Irf9 | GATGGGCAATAGCTCCCTGC | 3593 |
| Irf9 | GCCATAAGATGCCTCTTTAT | 3594 |
| Irf9 | GGGTTCAGGGATGAAGCTTG | 3595 |
| Irx1 | GTCGTGGGAGACTCAAAGAC | 3596 |
| Irx1 | GGGATTGCGTTTCTACAGCT | 3597 |
| Irx1 | GTGGACTCCCTGGTCAGGTC | 3598 |
| Irx1 | GCAAGGGTGTGGACTCAGTT | 3599 |
| Irx1 | GGCCTGGCTGCTCTGTTCTC | 3600 |
| Irx1 | GAGGAGAGCTCCTAGAGGGT | 3601 |
| Irx1 | GCTGAGAGGTCGCTGCCTAG | 3602 |
| Irx1 | GTTTACAGCTGTCTGACACC | 3603 |
| Irx1 | GCAAGCAAGTGTGCCTTAGC | 3604 |
| Irx1 | GTTGTGGGAGTAATGACAAG | 3605 |
| Irx2 | GGACTCTGAAACCTGGCGCG | 3606 |
| Irx2 | GGGTAGATGCTTGGCAGCCC | 3607 |
| Irx2 | GCTTCAAGGAGACACCTGTT | 3608 |
| Irx2 | GCTCTACCTAGCAAGCTTCA | 3609 |
| Irx2 | GTTGGAAACCAAGAGCAGTT | 3610 |
| Irx2 | GTCACGGATCTGGCTGCTGC | 3611 |
| Irx2 | GCTCTGAAGCTAGTAGAGGG | 3612 |
| Irx2 | GTCCAGGTCCCAGGGAACCA | 3613 |
| Irx2 | GCTATCTTCAGGGTGATGAG | 3614 |
| Irx2 | GCCTCAGCCGOGAGTACAGG | 3615 |
| Irx3 | GAAAGTCATACTGAAATTCC | 3616 |
| Irx3 | GAACGTGCTGCCTGGGAGTT | 3617 |
| Irx3 | GGGTTCGATGTCAGCATGTG | 3618 |
| Irx3 | GGTGCATCGGGAGTTGATTG | 3619 |
| Irx3 | GGTTGGCTTTAAGGTGAGCC | 3620 |
| Irx3 | GTGCCTGTTGGGAGAAAGAG | 3621 |
| Irx3 | GGCGACAGAGCCAGATTGCA | 3622 |
| Irx3 | GCTTTGTGCACTGTGCCTGT | 3623 |
| Irx3 | GGAATGGATTTCTTTCTCCC | 3624 |
| Irx3 | GCTACTTATCAGAACTTTGC | 3625 |
| Irx4 | GAAGCTAAGGCTCACGGGAG | 3626 |
| Irx4 | GACTCATTTCATGCTCACCG | 3627 |
| Irx4 | GCTCTGGAGCCTTCCATGGG | 3628 |
| Irx4 | GGGAAGCTGCCTTGCACAGT | 3629 |
| Irx4 | GGAGTCTTCAAGGGAGCGAA | 3630 |
| Irx4 | GCAAAGTCCAGGTGAGGAGG | 3631 |
| Irx4 | GATGGTCCGGAAGGGAGAAG | 3632 |
| Irx4 | GCCCTCACAATGCTATCCTT | 3633 |
| Irx4 | GCCTGGGTCTTTGTAATCTG | 3634 |
| Irx4 | GAGTAAGCTCCCGCCCAGAA | 3635 |
| Irx5 | GGCAAAGCTTGATCATTAGC | 3636 |
| Irx5 | GGGATGTGATTGTCATCCTG | 3637 |
| Irx5 | GGGCCGTTCGGGAACACAAA | 3638 |
| Irx5 | GATTACGTCATCCAGAGGAG | 3639 |
| Irx5 | GCAAGCAGTTTGCTCGGTTG | 3640 |
| Irx5 | GCCTCTTCTCGTCGTCTGCC | 3641 |
| Irx5 | GAAGCCACTGTGGAGCGTGG | 3642 |
| Irx5 | GTGTTCCGAGAACTCTGCCT | 3643 |
| Irx5 | GCCTCTCGGGCTGACCATTC | 3644 |
| Irx5 | GTCAGGTCTGTGGAGCCGGA | 3645 |
| Irx6 | GGCTGCACCTCGATCTGGAG | 3646 |
| Irx6 | GGCCAGGTCCTTGACCTCTT | 3647 |
| Irx6 | GCGTTCTCTGTGGTCCAAAC | 3648 |
| Irx6 | GATTCATTAAGTTAGTCCCT | 3649 |
| Irx6 | GAAGGAGTTTATTACACCAT | 3650 |
| Irx6 | GACAGGGCGACTGAAAGGTA | 3651 |
| Irx6 | GTCCCGGGAGCTCTTAGGGT | 3652 |
| Irx6 | GCTGCACCTCGATCTGGAGG | 3653 |
| Irx6 | GATTCTAACAACCAAGCGCC | 3654 |
| Isl1 | GAACTCAGTAATAGTAGGAT | 3655 |
| Isl1 | GTAGCATGCCCTTGTACGGA | 3656 |
| Isl1 | GTAGGTCCTTCCTGTGAAGC | 3657 |
| Isl1 | GCTTTCTAATTTGTTTCCTC | 3658 |
| Isl1 | GTCAACCTGGCTCTATAGAA | 3659 |
| Isl1 | GAATCTTATATAGGTGAGGG | 3660 |
| Isl1 | GCTCTCTGACATCCTATGTG | 3661 |
| Isl1 | GTTCCTCCTGAGCTCCCTGC | 3662 |
| Isl1 | GGGAGGAAAGGAACCAACCT | 3663 |
| Isl1 | GGGAACTGCTTCTCTGGGCT | 3664 |
| Isl2 | GGTGGGCCTGAGCCTTTGTT | 3665 |
| Isl2 | GTCCAGTGCTGGCATGAGAG | 3666 |
| Isl2 | GCCCGAGATCTATCTAATTC | 3667 |
| Isl2 | GGAAAGCCCTGGAGAAAGCC | 3668 |
| Isl2 | GTAATTTACCGTCTTCCCGG | 3669 |
| Isl2 | GAGAGGAGAAAGGAGAGGGT | 3670 |
| Isl2 | GAGATTGGCTGGGAGGAAGT | 3671 |
| Isl2 | GGCTTTCTCTAGGTAGGAGA | 3672 |
| Isl2 | GACGAGCTCTCTGTCATACA | 3673 |
| Isl2 | GCTCCCAGCAAGGGCAAGAA | 3674 |
| Isx | GGAGTGACAGGAGGAATTTA | 3675 |
| Isx | GTGTAACCAAGGAGGAGGGT | 3676 |
| Isx | GAGGTTTATAGGGTGAACCT | 3677 |
| Isx | GGTGTTGGGTGGAGAGCTGA | 3678 |
| Isx | GCTGTCTTGAAGACAGTAAA | 3679 |
| Isx | GCTCCAAGCCCAGAGTTTAC | 3680 |
| Isx | GAGCCTCACCCATCACACCC | 3681 |
| Isx | GTCTTACTAGTACAAATCCA | 3682 |
| Isx | GAAACCGAGGCTCAGAACAA | 3683 |
| Jun | GAGAATAAAGTGTTGTGCCG | 3684 |
| Jun | GTTTGGCTGTCTAGTGACGG | 3685 |
| Jun | GATATGACTCCACCAGTGAG | 3686 |
| Jun | GTAAGTGCGTTGAAGTTGAG | 3687 |
| Jun | GAGGAACTCGGTTTCCATTT | 3688 |
| Jun | GGGCTGCGGAGAGAGGAACT | 3689 |
| Jun | GAGGTTTGCTTGGCGAGGGA | 3690 |
| Jun | GCTGGAAGCTCAGTTGGGAA | 3691 |
| Jun | GCAAGCCAATGGGAAAGCCT | 3692 |
| Jun | GCAAATCAGGGAGGGAGGAA | 3693 |
| Junb | GTGTCTGCAGGAGACTAACC | 3694 |
| Junb | GGACTGTTCCATTGGCCGGC | 3695 |
| Junb | GGTAATCGGAGTAGAAAGAT | 3696 |
| Junb | GCTAGGCCAAAGCCAAGTCC | 3697 |
| Junb | GAAGAGAAGAGTGGGAGGCT | 3698 |
| Junb | GGTCTCTGGTAAGATAAAGG | 3699 |
| Junb | GGTGAGTCAGTGTGGTCTCC | 3700 |
| Junb | GGAAAGGGCCAAGACACAAC | 3701 |
| Junb | GGTGACTAAGGGAGGGCTTT | 3702 |
| Junb | GTAAACAGCGGCCACGAGCC | 3703 |
| Jund | GGAGCCTGCAAATGAGAATC | 3704 |
| Jund | GGCAACAACTGGTCAAGGCT | 3705 |
| Jund | GCGAAGGTCCTGAGGTGCAA | 3706 |
| Jund | GCTCCTGCTGATGGAAGTTC | 3707 |
| Jund | GGTGCAAAGGAGCTCCAATG | 3708 |
| Jund | GAGTGTGAGGGCAGAGCTTG | 3709 |
| Jund | GCTGGGAACGGAGGTGGAAG | 3710 |
| Jund | GGATCTCTCACCTTCCAGCT | 3711 |
| Jund | GCATACTGTACTAATTAAGA | 3712 |
| Jund | GCAACCAAGTTTCCAAATAA | 3713 |
| Kat2a | GCTGTTGGTAGTTCTGATGG | 3714 |
| Kat2a | GTGTCTGAAGTGACCTGTGA | 3715 |
| Kat2a | GACCATCAGCTGATTCTGAA | 3716 |
| Kat2a | GGCCAGAATCTGACGGTGAC | 3717 |
| Kat2a | GCCCTTCTGGATGGGAAGAG | 3718 |
| Kat2a | GTGACAATTACTATCCTCTT | 3719 |
| Kat2a | GCCTGATCAGCTGCCAGAGA | 3720 |
| Kat2a | GCCTGCCCTATTGTGGCTGC | 3721 |
| Kat2b | GGGTGGTATGCTTATGCTCT | 3722 |
| Kat2b | GGAAGACCAAGAATGAGCAA | 3723 |
| Ktt2b | GTTTGCAGAGTGAATGCTGA | 3724 |
| Kat2b | GCATTCACTCTGCAAACATT | 3725 |
| Kat2b | GTGGAAGTAAACAGGAGTGA | 3726 |
| Kat2b | GAGTCATCTCCCTCCCTCTC | 3727 |
| Kat2b | GAACCGAATATGACCAGAGA | 3728 |
| Kat2b | GTATCTCATGGAAGAATTCC | 3729 |
| Kat2b | GAGGAGGATTGGCCGCTGAC | 3730 |
| Kin | GTTGTATATTAGAATGCCCA | 3731 |
| Kin | GGCGCTCCAGCACTGAACTA | 3732 |
| Kin | GGGAGCAGCGTGACCCTTTA | 3733 |
| Kin | GCCTTGAAGGTCGGGCAGAC | 3734 |
| Kin | GGTGCTAGAGACTCACCTCA | 3735 |
| Kin | GTGAACCTCTCGAGCCTTTA | 3736 |
| Kin | GTTTCTGTACCAGCCTCCAG | 3737 |
| Kin | GTGTTCACTAGTTAGCTAGT | 3738 |
| Klf1 | GCCTAACGGCTCATTGTGTG | 3739 |
| Klf1 | GACCCTCACTGGCTACTGCA | 3740 |
| Klf1 | GTGCCCTATGAGTCAGGGTA | 3741 |
| Klf1 | GGGTGCTGGTGGTTGTCTAG | 3742 |
| Klf1 | GGCTACTGCAIGGAGCTGAA | 3743 |
| Klf1 | GGTGCCCTATGAGTCAGGGT | 3744 |
| Klf1 | GATAATGCCTGAAAGGGAGC | 3745 |
| Klf1 | GGGTGTGTGCGATATGTGTG | 3746 |
| Klf1 | GTCTGGGTGGCTAAATAGAC | 3747 |
| Klf10 | GAGTGATCACAGCAGGAAAG | 3748 |
| Klf10 | GGGTAGGAGAAACTGGGTAG | 3749 |
| Klf10 | GAAGGACAGTGCTTATTGAA | 3750 |
| Klf10 | GTACCAAAGAGCTAGTGGCG | 3751 |
| Klf10 | GCGGTTTCTTGGTAGGGCGT | 3752 |
| Klf10 | GGAGAACCAGGGCGAGATGG | 3753 |
| Klf10 | GGAGGACTGAAGGCTAGGGT | 3754 |
| Klf10 | GGGCGGAGGACTGAAGGCTA | 3755 |
| Klf12 | GATTTGACCATCTCTTGCCG | 3756 |
| Klf12 | GAGTCACATTGATCCTGCAA | 3757 |
| Klf12 | GGCTGTATAGCTCTTCACCA | 3758 |
| Klf12 | GAAAGTTGCAGGTCATGTTA | 3759 |
| Klf12 | GCAATCAGCTCTAACTTCTT | 3760 |
| Klf12 | GGAGTAGGGAAATGCAAGCC | 3761 |
| Klf12 | GCTGTATAGCTCTTCACCAA | 3762 |
| Klf12 | GGGAAGCCACCTGACGATGG | 3763 |
| Klf13 | GAGAGGGTTCTACTGGCCGC | 3764 |
| Klf13 | GGATATTTCTATCTGGGTTT | 3765 |
| Klf13 | GGTGAGGAGGTGGCTGGAGA | 3766 |
| Klf13 | GTGTGTTAAGATTGGTTCAA | 3767 |
| Klf13 | GTTTGAAGCCTCCAGGACCG | 3768 |
| Klf13 | GCCACAGACAGTCATCTCAT | 3769 |
| Klf13 | GATAGAGACAGTCTCTCCTC | 3770 |
| Klf13 | GGTGGCGTATCGGTCCCTAT | 3771 |
| Klf13 | GTCTAGACTTTAGAGCAAGG | 3772 |
| K1f13 | GCCACCAGAGAACTCCGCTG | 3773 |
| Klf16 | GGTGCTCTGCTGGTACACGA | 3774 |
| Klf16 | GGTGGCAGAGGTCCTTGCTC | 3775 |
| Klf16 | GTGCTCTGCTGGTACACGAG | 3776 |
| Klf16 | GAAGCTGAACCAGGCTTCAT | 3777 |
| Klf16 | GGGCTGCTACATGCAATGGC | 3778 |
| Klf16 | GGAACCCAAAGTTCTCAACG | 3779 |
| Klf16 | GGAGCGATTGGAAACTTCCA | 3780 |
| Klf16 | GACCTCTGCACAAATCTAGC | 3781 |
| Klf16 | GGGACCATCATTTCACAACT | 3782 |
| Klf16 | GCCTTGGGAGGTGACGATCC | 3783 |
| Klf2 | GAGGAAGTATGTGGTGAGCC | 3784 |
| Klf2 | GCTGGCTCAGTGCTTAAGAG | 3785 |
| Klf2 | GAGGGTAATAGAGAGAGGGA | 3786 |
| Klf2 | GCACTAGAAGGATTTATGTG | 3787 |
| Klf2 | GTATGTTTGTGGGAGGTGAA | 3788 |
| Klf2 | GCAAGAGGGTAATAGAGAGA | 3789 |
| Klf2 | GCGGTATATAAGCCTGGCGG | 3790 |
| Klf2 | GGCGACGGCGTCAACAAACC | 3791 |
| Klf2 | GACGGAAACGCGTCCCGGAT | 3792 |
| Klf3 | GTTTCTTGGGTGACTCAGTT | 3793 |
| K1f3 | GACGTAGGGACAGGGCATCC | 3794 |
| Klf3 | GTGGCCTCACGCAGCCTTTC | 3795 |
| Klf3 | GACCTTTCTATCTGTACCGA | 3796 |
| Klf3 | GCCTGGGCTGTTTAGGAAGC | 3797 |
| Klf3 | GATGCCCTGTCCCTACGTCG | 3798 |
| Klf3 | GAATGAATGGTAAGAGGGTA | 3799 |
| Klf3 | GAGGATTTAGATAAGCCGGA | 3800 |
| Klf3 | GAACAGGACATTCGTCATGA | 3801 |
| Klf3 | GCTTGGTGCTAGGCTAGAGT | 3802 |
| Klf4 | GGATGACTGCCCAGCTGTGG | 3803 |
| Klf4 | GGCGTTCCAGATTTACATTG | 3804 |
| Klf4 | GAAAGGGATGAGTTGTGAGC | 3805 |
| Klf4 | GGGACCCTAGTGCTCCAAAG | 3806 |
| Klf4 | GGCAGTAGCCAGAGCTAGGG | 3807 |
| Klf4 | GTGCGTATGCGAGAGAGGGC | 3808 |
| Klf4 | GCAGTTGGCAGATGATGTAA | 3809 |
| Klf4 | GATAATGGAAGGAACAAGGA | 3810 |
| Klf4 | GAATCTCAGAAGCTAGGAGA | 3811 |
| Klf4 | GACAAGCGCGTACGCGAGCA | 3812 |
| Klf5 | GTGCTCAAATAACTCTGAGA | 3813 |
| Klf5 | GTCAACAGCGGTGTTTGTCT | 3814 |
| Klf5 | GTAATTTCTGGCATAGAGAT | 3815 |
| Klf5 | GGGCTGCAATCCTCTTTCTG | 3816 |
| Klf5 | GTGAATGTTTGTGCCTTCTT | 3817 |
| Klf5 | GCGGGTGGAATCTAGGAAGA | 3818 |
| Klf5 | GTGCAGCCAGCCAGTGTGAA | 3819 |
| Klf5 | GAGAGGAGCGGGTGGAATCT | 3820 |
| Klf5 | GGCTAGGAGGGTAAGCATAG | 3821 |
| Klf5 | GGAAGGTGAGTGGTTTGGTT | 3822 |
| Klf7 | GTCCTCTCCGAGTGCCGCAT | 3823 |
| Klf7 | GCAAATGGCAGTAAAGGCCT | 3824 |
| Klf7 | GTTTGGTGGAACCCACACTC | 3825 |
| Klf7 | GTGACCATGTAAGGTAAACA | 3826 |
| Klf7 | GTACCCAGTGCAGATCCGAG | 3827 |
| Klf7 | GTAACTTCATGGAGCAGGTA | 3828 |
| Klf7 | GACCATGTAAGGTAAACAGG | 3829 |
| Klf7 | GGCACTCGGAGAGGACCATG | 3830 |
| Klf7 | GACCATGAATTTGAGAGGGA | 3831 |
| Klf7 | GATCTCCCTGCTGCCTTACA | 3832 |
| Klf9 | GAGAGGTGCGTCTAGAACTA | 3833 |
| Klf9 | GAAGGGCCCTTCTGACTGGC | 3834 |
| Klf9 | GGATGGGCGTAACTGCCTAG | 3835 |
| Klf9 | GGGCCTGGATTGTGACGTGA | 3836 |
| Klf9 | GGGATGGGCGTAACTGCCTA | 3837 |
| Klf9 | GGCTCCTCTAAAGCAGAGTT | 3838 |
| Klf9 | GCACTCCTCCCTGTTCCTGC | 3839 |
| Klf9 | GGCTACCAAAGATTAAGGGC | 3840 |
| Lbx1 | GGCAGAAATGCTGATAGTAG | 3841 |
| Lbx1 | GATCCTCCCTATAGGCAGAG | 3842 |
| Lbx1 | GTGTTATAACAGGGAAGGGC | 3843 |
| Lbx1 | GCAAGATTGCAGAAGGAGGT | 3844 |
| ibx1 | GGGAGTGGGACAGAAAGAAT | 3845 |
| Lbx1 | GGAAGGAACAAGAGGGAGAA | 3846 |
| Lbx1 | GAGATTGGGAGGTGGGAGGC | 3847 |
| Lbx1 | GTACCCTGTGCCCTCCTTCA | 3848 |
| Lbx1 | GAACAGGCTTCTTCGGTGCA | 3849 |
| Lbx1 | GTAAGAACTGGAGCCCAGGC | 3850 |
| Lbx2 | GGCCTCAGAATCAGAGGGAA | 3851 |
| Lbx2 | GCATATTAAGTGAAACCACA | 3852 |
| Lbx2 | GAGTCCAGTCCTCACTAGCC | 3853 |
| Lbx2 | GGCTGGTACGACTTGCTCAG | 3854 |
| Lbx2 | GGCTCAGGTAAGGAAGGGAT | 3855 |
| Lbx2 | GGCTCCTGGCTAGTGAGGAC | 3856 |
| Lbx2 | GCTGCTGTCACTGAGCTGAC | 3857 |
| Lbx2 | GGTCACTCACATCTCCTATT | 3858 |
| Lbx2 | GAGCTGAAATAAGGCAACTC | 3859 |
| Lbx2 | GGAGAGGTTGCAGTGTCTGT | 3860 |
| Ldb1 | GACTCTGACCTATCATTCAA | 3861 |
| Ldb1 | GGGCAAGTGGTCCCAGGACT | 3862 |
| Ldb1 | GTCAAGTCCTTCTATGCCCT | 3863 |
| Ldb1 | GGGACACACTCACATGGCAA | 3864 |
| Ldb1 | GATTCCGATCACCTACCTGG | 3865 |
| Ldb1 | GTGGTGCTGTCAGGGTAAGG | 3866 |
| Ldb1 | GGAAACACACACGCACAGGC | 3867 |
| Ldb1 | GGCTGTCATACAGCTCAAGA | 3868 |
| Ldb1 | GGAAGAGTTCTTCCCTTCCA | 3869 |
| Ldb2 | GGGAAGGGTGTTCCCTAGAA | 3870 |
| Ldb2 | GTACCTGCTGTACTTCGGAT | 3871 |
| Ldb2 | GAAGAGGTAAATACAAACTC | 3872 |
| Ldb2 | GGAGCACAGCTCTCCCTTTG | 3873 |
| Ldb2 | GATGGTTCCACATTCAGGTC | 3874 |
| Ldb2 | GTGCCAGTGTTGTTGTGTTT | 3875 |
| Ldb2 | GGGTGGATGTTTCTTGCAGG | 3876 |
| Ldb2 | GCTAAGTCAGCGGGTTTAAG | 3877 |
| Ldb2 | GTGCACAGCTGACCCAAAGG | 3878 |
| Ldb2 | GCCCGGAGGAATCTTCCAGA | 3879 |
| Lef1 | GGGTGCTAGGAAATGAACTA | 3880 |
| Lef1 | GTCGCCAGTGCTATGCCTCT | 3881 |
| Lef1 | GAACTCTAGCGAACCACTGG | 3882 |
| Lef1 | GTAGAGTAAATAGAGACACG | 3883 |
| Lef1 | GAGCATTTAATCTGCTGGAG | 3884 |
| Lef1 | GGAGGGAGTCTGTTAGGAGG | 3885 |
| Lef1 | GACTAGAAGTGAGGCGCCGG | 3886 |
| Lef1 | GGGCAGAAAGTTGCCATTTA | 3887 |
| Lef1 | GATTGGGCGAGTGGGATCCT | 3888 |
| Lef1 | GAAGGAAAGAAGCTCTAACG | 3889 |
| Lef1 | GACTTGTTCTAGGAAGTGTT | 3890 |
| Lhx1 | GGTATTAATCGACTTGTTCT | 3891 |
| Lhx1 | GGGTGGGAGAAAGAGTGGGT | 3892 |
| Lhx1 | GGTGAAGTAACCCAGCAGCG | 3893 |
| Lhx1 | GCGAGATCTGGAAGCTTGGG | 3894 |
| Lhx1 | GACTTTGAAGGATGGAGGGT | 3895 |
| Lhx1 | GGGTGGACTTTGGATGGACA | 3896 |
| Lhx1 | GCTCGAGTCTAAGGAGAGGT | 3897 |
| Lhx1 | GACCTTCCCACCTAAAGGGC | 3898 |
| Lhxl | GGACTGCACCGTAGCAGCAG | 3899 |
| Lhx2 | GACGCAATAGTGTCTATTGG | 3900 |
| Lhx2 | GTGAAGCAGGGTATGGAAGC | 3901 |
| Lhx2 | GGTGTCTGGTGGAACAGGAA | 3902 |
| Lhx2 | GACTGCCCTTGGTTTCTTAG | 3903 |
| Lhx2 | GTAACCGTGCCCAAGAGGCA | 3904 |
| Lhx2 | GCAGGATGTGCCAGTGGCTC | 3905 |
| Lhx2 | GAGTGGGAGAGCTAGTGGGA | 3906 |
| Lhx2 | GACGTCGCTTTGCCCTGTCC | 3907 |
| Lhx2 | GTGACTTGTCCGAAGTCCCA | 3908 |
| Lhx2 | GTGCCTGACACCTACTTACC | 3909 |
| Lhx3 | GTCTAACATGGAGGCTGGGA | 3910 |
| Lhx3 | GTGGGTTCAGAGACAATCTG | 3911 |
| Lhx3 | GAAAGGTGCACAGTCTCCAG | 3912 |
| Lhx3 | GTGAGGACAAGGTAACAGCA | 3913 |
| Lhx3 | GTAGTAGGAGCCCTCAGTGA | 3914 |
| Lhx3 | GATGTGGAAATCCAGGTGCA | 3915 |
| Lhx3 | GGTCACAGTCCTAGGGATGG | 3916 |
| Lhx3 | GGTCCAGAGTGTCAGAGTTG | 3917 |
| Lhx3 | GCTTCTAGGCACCTCGGTTC | 3918 |
| Lhx3 | GCACCTCGGTTCCGGCTGAA | 3919 |
| Lhx4 | GCTACACTGGTTTGTTTGGT | 3920 |
| Lhx4 | GATGGGTCTTTACAACCAAA | 3921 |
| Lhx4 | GAAACCTACCGGGTCAGCCC | 3922 |
| Lhx4 | GAACTCGGAGCGCCAACCCA | 3923 |
| Lhx4 | GGTGGCTGTGTGTGCTACTT | 3924 |
| Lhx4 | GCAACAGTGTCTCCTCAACC | 3925 |
| Lhx4 | GCCCGGGAGAGCGAGATCAA | 3926 |
| Lhx4 | GTATAAATACTGCGGCGGGC | 3927 |
| Lhx4 | GCCCGCCGCAGTATTTATAC | 3928 |
| Lhx4 | GTCCTCTAGGATCAAGGAGG | 3929 |
| Lhx5 | GCAGGTGTGTGGGTACCAGC | 3930 |
| Lhx5 | GGGATTCTCCTCATGGATTA | 3931 |
| Lhx5 | GGCCATCTGTCAGTGCTGTT | 3932 |
| Lhx5 | GATTCTCCTCATGGATTAGG | 3933 |
| Lhx5 | GTCTTGGCACAATTCCTCTA | 3934 |
| Lhx5 | GACTCTGAAGGGCTGTGTGT | 3935 |
| Lhx5 | GTTTGTGTGTGTGTGTGTGG | 3936 |
| Lhx5 | GCGAAGCTGCCTTTGGCTCT | 3937 |
| Lhx5 | GGTAAATACTTACTTAGCTT | 3938 |
| Lhx5 | GIGACATCCCTGAGTCAACC | 3939 |
| Lhx6 | GTGTTTGAGGAAGAAGGCTG | 3940 |
| Lhx6 | GCTGTTTACATCTGTAAATG | 3941 |
| Lhx6 | GGTAAATCTTGAAGTGGAAG | 3942 |
| Lhx6 | GATGAGATTTACATAGTCTG | 3943 |
| Lhx6 | GGAGCCTGTGCTAGTGAGAG | 3944 |
| Lhx6 | GCTAGTGAGAGTGGGAGGGT | 3945 |
| Lhx6 | GATACTACTTCAGATTCTTC | 3946 |
| Lhx6 | GGTCAGCCCATCTACAAGGC | 3947 |
| Lhx6 | GAACTCAGTCACGTAAGTGG | 3948 |
| Lhx8 | GAAATTTCAGTCCAATAGGA | 3949 |
| Lhx8 | GCTTCCGGGCTTAGAGAAGG | 3950 |
| Lhx8 | GCTCTTTCAGCGGCTCACGG | 3951 |
| Lhx8 | GATAATGAAGGGACAAACGA | 3952 |
| Lhx8 | GGGTTTGGGCTGGAGATGGG | 3953 |
| Lhx8 | GGAACCTCGCAGAGAGGAGG | 3954 |
| Lhx8 | GCCTTTGATAGGAATCGCCA | 3955 |
| Lhx8 | GAATGCTGGCTCCAGCAGGT | 3956 |
| Lhx8 | GGGAAAGGAAGTGCCGGAGC | 3957 |
| Lhx8 | GACACAGGCAATTATGCTGC | 3958 |
| Lhx9 | GCAAGGCAAAGGCAGGCTAG | 3959 |
| Lhx9 | GTGGCCTCAGAACGGGTGTC | 3960 |
| Lhx9 | GGCATGGACCAAGGACTGGA | 3961 |
| Lhx9 | GGGTTTCTAATGCCCAGCTA | 3962 |
| Lhx9 | GAGGCTACAGTGTCTCAGCT | 3963 |
| Lhx9 | GGATTATTGAGAGGCTGGCA | 3964 |
| Lhx9 | GAAAGGTGGGAATGAAGCAG | 3965 |
| Lhx9 | GTCTATGCGGCTCTGAGTGT | 3966 |
| Lhx9 | GCAGGAAGTCTTTGGAAAGG | 3967 |
| Lhx9 | GAAACTAGGTACTGGAGCAG | 3968 |
| Lmo1 | GTGCTGCCCAGCAAGTCTCC | 3969 |
| Lmo1 | GGCAGGGAAGTCAGGCTTTG | 3970 |
| Lmo1 | GTGCAAACCTCATACATTGA | 3971 |
| Lmo1 | GAGTCTAGGAGGAGAGGCAC | 3972 |
| Lmo1 | GTGCCTCAGGCTTGGGAAGC | 3973 |
| Lmo1 | GCTCTAGAATATCTGGGATG | 3974 |
| Lmo1 | GGCATCTTAGGATTCCACCC | 3975 |
| Lmo1 | GCTGCACCAGTTGGGCTGAG | 3976 |
| Lmo1 | GTGGTCTCTCTTAAACTTAT | 3977 |
| Lmo1 | GAGCTCTAGAATATCTGGGA | 3978 |
| Lmo1 | GTAGATTTCACATACTAAGA | 3979 |
| Lmo2 | GGGAGATACTTTCATGACTT | 3980 |
| Lmo2 | GTTCAGCTGAGTTCACATGA | 3981 |
| Lmo2 | GGTACCTTCTTCAAGCACCC | 3982 |
| Lmo2 | GGGCTGTTCTTACTAAACAA | 3983 |
| Lmo2 | GAGTGGTTACTTTCAGCCTG | 3984 |
| Lmo2 | GGAGGACTTTGCTCAGTACG | 3985 |
| Lmo2 | GTCTTTCACAACTCTTTGGA | 3986 |
| Lmo2 | GCTATTGCTAGGGAGAAATC | 3987 |
| Lmo2 | GAACTTGTCTTCAAGCTTGA | 3988 |
| Lmo3 | GGTCCAGTTGGTTTGGGACT | 3989 |
| Lmo3 | GTGTGATAGGCATGGGTGGG | 3990 |
| Lmo3 | GAGCTCCAAAGGAGAAGGGT | 3991 |
| Lmo3 | GAAATGCATTAAAGCTGACA | 3992 |
| Lmo3 | GACCGGCTATGCCAGGACTT | 3993 |
| Lmo3 | GAGCTGTTCATTTAATTCCA | 3994 |
| Lmo3 | GTGGGCGAGTCCTGGAGGTA | 3995 |
| Lmo3 | GCAGTAGCATAGAGTCACCA | 3996 |
| Lmo3 | GATCCCTGGAGAACAATACA | 3997 |
| Lmo3 | GCTTTGTTGCTAATTTCCCA | 3998 |
| Lmo4 | GGTCTGGTIGGTCTTTGTGG | 3999 |
| Lmo4 | GAGAAACACTAGGACTTTAT | 4000 |
| Lmo4 | GAGCAGATAGCTGGGAGCCT | 4001 |
| Lmo4 | GCAAATGCTCGCATCGCTTT | 4002 |
| Lmo4 | GAATGTCTTGAGCAGATAGC | 4003 |
| Lmo4 | GGCTTTATCTGGGATCCATT | 4004 |
| Lmo4 | GCAGTTTAAAGACCTAGGGC | 4005 |
| Lmo4 | GAATCTGCATTTCCTGCCCT | 4006 |
| Lmo4 | GAAACTTACTTTCCCAGAAA | 4007 |
| Lmo4 | GAGCTCTGCCTAGGGAAGTG | 4008 |
| Lmx1a | GTTCGCTCCTGCTCTCTCCC | 4009 |
| Lmx1a | GCCTCTCTAGAGGCAGGAAC | 4010 |
| Lmx1a | GTCTGCCATCCAGATAGAAC | 4011 |
| Lmx1a | GATGTGTTTATTGAGTCACT | 4012 |
| Lmy1a | GGGAACGTCTGCAGGAGCAA | 4013 |
| Lmx1a | GTTAGGAGAACGCAGTTAGG | 4014 |
| Lmx1a | GAGTACCATAGTTCTAGTGG | 4015 |
| Lmx1a | GGCCACATTAGTATAGGATG | 4016 |
| Lmx1a | GGGTTAGGAGAACGCAGTTA | 4017 |
| Lmx1a | GGGCAGCAGACTGGAGCATC | 4018 |
| Lmx1b | GACAGCCTGGTGTGCTGAGA | 4019 |
| Lmx1b | GGATCTGGACCGCCTTCTCT | 4020 |
| Lmx1b | GGATCAGATTTGGAGCCTGA | 4021 |
| Lmx1b | GGCCTGGCAGAAATAGGGCG | 4022 |
| Lmx1b | GAACGCAGCGACTTCTCCAG | 4023 |
| Lmx1b | GAGCCGCTCGGTTTAGAGCT | 4024 |
| Lmx1b | GGCGACGGCACTATTTGACG | 4025 |
| Lmx1b | GGTCCCTTAGCCACAATGAA | 4026 |
| Lmx1b | GAGACCAAGAGAGTGTTAAG | 4027 |
| Lmx1b | GCTCCTAGGGTCGAGGGATG | 4028 |
| Lrrc41 | GGTCAACCAAAGAATTCTGA | 4029 |
| Lrrc41 | GGCTCCCGACATGGGACTAG | 4030 |
| Lrrc41 | GACTAGTAAGGGTCACTCGA | 4031 |
| Lrrc41 | GCATTTGTCTTGTCTACTTC | 4032 |
| Lrrc41 | GCTTTGTTGAGCTAGGTCCC | 4033 |
| Lrrc41 | GGACCGCTCCATAAGGGATA | 4034 |
| Lrrc41 | GTAAAGAGCAGAGGTTACAG | 4035 |
| Lrrc41 | GGGCTCCTGGGATCAAACTC | 4036 |
| Lrrc41 | GCCCAATTTGTGCGTGTGTT | 4037 |
| Lrrc41 | GTTAGTCTTTAACGTAGCTT | 4038 |
| Lyl1 | GGAGGAAATGCCTGGATAGC | 4039 |
| Lyl1 | GGCTGGGCAAAGACAAAGTG | 4040 |
| Lyl1 | GAAGGAGCCAGCTGAGGACC | 4041 |
| Lyl1 | GCTCAGGAGAGCAGTTCATC | 4042 |
| Lyl1 | GGCCTCAGAGGACCGGAAAG | 4043 |
| Lyl1 | GCTAGAGGAGTCACTAGGGT | 4044 |
| Lyl1 | GCAGTTCATCAGGTGGCCAC | 4045 |
| Lyl1 | GTGGTAATGTTGTAGAAGTG | 4046 |
| Lyl1 | GCTCCGGAAGGAGACAATTC | 4047 |
| Lyl1 | GCTGTGCTAGAGGAGTCACT | 4048 |
| Maf | GTTATTGCCACAAATCGGGT | 4049 |
| Maf | GCTCTTTCAAAGGGCTGGCA | 4050 |
| Maf | GGATTCTAGTGTACATTCGA | 4051 |
| Maf | GTGCGAAGTTTAGTGCACCA | 4052 |
| Maf | GGAGGATGGTTTGCTTTCCT | 4053 |
| Maf | GATCACCTCACTTGCAGAGA | 4054 |
| Maf | GTGTGCACGTTCGAGCTTTC | 4055 |
| Maf | GGGTTTCCGGACTTGTCCGG | 4056 |
| Mafa | GATCCCAACCGAAGATAGAA | 4057 |
| Mafa | GGAGGAGGAGGGCAGGATTG | 4058 |
| Mafa | GACCTCGTGCTCTAACTCAA | 4059 |
| Mafa | GTCTCCTTTGGAACAGGCTG | 4060 |
| Mafa | GCTGTGGTTCATCTAGGACA | 4061 |
| Mafa | GGGATCTGGAATTCTGGAGG | 4062 |
| Mafa | GGACACTGAGGGAAGGAGCT | 4063 |
| Mafa | GGCCTGGAGTCTCCAGAATG | 4064 |
| Mafa | GAGGAACAGAAGGAGGAGGA | 4065 |
| Mafa | GGTGTCTCAGATCCATTAGG | 4066 |
| Mafb | GGAGGCTGGACCATTGAAAT | 4067 |
| Mafb | GGTTTAGATCAGTGAACTGC | 4068 |
| Mafb | GGTGAGTGTGTCCTAGCTGC | 4069 |
| Mafb | GGAGGAGGAAGGCAGAACAC | 4070 |
| Mafb | GAGCCACTGAGTGCACAGAC | 4071 |
| Mafb | GTGGCAGCCTGGAGAGAGAA | 4072 |
| Mafb | GCAAACCCTCCTGGGAACAC | 4073 |
| Mafb | GTGGAAACCTTACAACTCCG | 4074 |
| Mafb | GTTGCGCACCGTGGCCACTT | 4075 |
| Mafb | GCGGGCCGAGTGAATGTGTG | 4076 |
| Maff | GTAAGGACGCGTCAGGGACG | 4077 |
| Maff | GATCGGGACCGCAGTTCACT | 4078 |
| Maff | GCAAGAACTCCGAGGTTTCA | 4079 |
| Maff | GGTTTCACGGGTCCTGGGTC | 4080 |
| Maff | GGTTTGTTTACGTCTCCCGG | 4081 |
| Maff | GGTGACGTCACTGCATGACT | 4082 |
| Maff | GCTCGCCTTACAACTGCGCG | 4083 |
| Maff | GACAAGCACGCACTGAGCGC | 4084 |
| Maff | GAAACAAGGCTACCAGACCC | 4085 |
| Maff | GCTCTGAAGCCTCTTCTCCC | 4086 |
| Mafg | GACCTGTGAGTTGGAGGCAA | 4087 |
| Mafg | GGCTGATCCTTGCTTGCTGT | 4088 |
| Mafg | GGGCTCTGGACCACTCATTC | 4089 |
| Mafg | GACCGTGCTCCTGCAGAGAC | 4090 |
| Mafg | GCACAGGAAAGTGCAGAGTG | 4091 |
| Mafg | GGTGTATGTGTGTTGAGGGT | 4092 |
| Mafg | GCCTCAGGGCTCAGGGTTAA | 4093 |
| Mafg | GGAGAACGGCTCAGGAAGGG | 4094 |
| Mafg | GGAGAGAAGACCTACGTAGG | 4095 |
| Mafg | GCCATTCAGGGTCACAGAGA | 4096 |
| Mafk | GGTGGTGGCAGTGAGGATGA | 4097 |
| Mafk | GTCAGGTTAGAGGCAGAGGG | 4098 |
| Mafk | GGAAGGTGCCTGGAAGAAGG | 4099 |
| Mafk | GGACTGCCAGGATGTCGTGC | 4100 |
| Mafk | GGTGAAGGCACTTAGGGTGA | 4101 |
| Mafk | GTTTCTGGTCTCCCAGAATG | 4102 |
| Mafk | GAGGCTGACAGCAGGGTGCA | 4103 |
| Mafk | GTGCTGAGGAACTGCTTCCG | 4104 |
| Mafk | GTAAGGAGGGAGGAGGGATT | 4105 |
| Mafk | GACATCACTAATGTTGTTAT | 4106 |
| Mapk8ip1 | GCTTTGTAGCCAGGATGGGT | 4107 |
| Mapk8ip1 | GTGTCTATGTCCTCTCAGCA | 4108 |
| Mapk8ip1 | GATCTAGCCCGTGGTGGCTA | 4109 |
| Mapk8ip1 | GGATCGAAGCGTCAGCACTT | 4110 |
| Mapk8ip1 | GGAGAACCACACAGCCTGGC | 4111 |
| Mapk8ip1 | GAGTCCCAGACCTTACAGGC | 4112 |
| Mapk8ip1 | GTCCTGCTCCATTTATGTGA | 4113 |
| Mapk8ip1 | GAACCTAAAGCCAGAGGCCT | 4114 |
| Mapk8ip1 | GCTCCATTTATGTGAAGGGC | 4115 |
| Mapk8ip1 | GACGGAGGAGGTCACTACCA | 4116 |
| Max | GGACACATCATGCCATTCCT | 4117 |
| Max | GTTTCTGCACTCAATAGTCA | 4118 |
| Max | GCCAGATTTCAGGGAGGGTG | 4119 |
| Max | GACTTGTAGTCCTCGAGCGT | 4120 |
| Max | GAGAAACTACAAATCCCATC | 4121 |
| Max | GAGATGCCAGATTTCAGGGA | 4122 |
| Max | GATACCAGAAGTAGAGACAA | 4123 |
| Max | GAATCTAGTTTAGGCTTTGT | 4124 |
| Max | GGCTGTAAGGGAGACAAAGA | 4125 |
| Maz | GGAAGGCATCTCTGGGAAGC | 4126 |
| Maz | GGGACAGGAGGGACTCTAGA | 4127 |
| Maz | GGGTTGTTACCTCACTGAAG | 4128 |
| Maz | GAAGGGAGTGGACACAGCAC | 4129 |
| Maz | GGGTGGATCAAGCTCTCTGC | 4130 |
| Maz | GAGGACTTGGAACAGGTGGA | 4131 |
| Maz | GTTGCTGGGATCCATGGCGG | 4132 |
| Maz | GAAATAACGGCCGCTGGCGG | 4133 |
| Maz | GACACACAAGAGGCTGGAGC | 4134 |
| Maz | GCAGCCAATCCAAACACAAG | 4135 |
| Mbd2 | GCCTGTCTCAGAGATGAGTG | 4136 |
| Mbd2 | GTGTACAGATGGAGAAACCA | 4137 |
| Mbd2 | GAGTGGCAGAAGTGTACAGA | 4138 |
| Mbd2 | GACCAGTGACCTTCATGCAG | 4139 |
| Mbd2 | GTGTCGTGAAGGCAGAGGCT | 4140 |
| Mbd2 | GGCTCTTGATATAAACCTCC | 4141 |
| Mbd2 | GTGGCCCTGACTCCAAGGTC | 4142 |
| Mbd2 | GGGAGTTTGTGCAGGAGTGG | 4143 |
| Mbd2 | GCAAACAAAGGCTCTGAGCT | 4144 |
| Mecom | GATTCTCAGGCAGGGCTCTA | 4145 |
| Mecom | GACCAGTTCACTGAAAGATG | 4146 |
| Mecom | GGCAGTTCTCTTGCCTAGTG | 4147 |
| Mecom | GTAGTTTGGAAGCTCTGAAG | 4148 |
| Mecom | GGCTTCCCTGCATTGATCTT | 4149 |
| Mecom | GTGTTTCTGTCTTCTCTTGG | 4150 |
| Mecom | GATGGCAATCGCCGAGGAGG | 4151 |
| Mecom | GTGGTGGGTATTCTTAGATG | 4152 |
| Mecom | GTTACTATTGGAGAGAGGCA | 4153 |
| Mecom | GGGAAGTGAGAAGGGTGGAT | 4154 |
| Mef2a | GATACGAGATTACCAGACAC | 4155 |
| Mef2a | GAGGGTTTGTGCCCATTGCA | 4156 |
| Mef2a | GATGTGCACAAAGCAGCCAT | 4157 |
| Kaf2a | GCAAACAGAAGGCAGGGATG | 4158 |
| Mef2a | GAAGTTACAAAGGAAGCTGG | 4159 |
| Mef2a | GCTGGATCCTTGCTGTGACC | 4160 |
| Mef2a | GCCCGGGAGAGAAGAAAGAG | 4161 |
| Mef2a | GGTAATAAGAATGTGATGGC | 4162 |
| Mef2a | GAGGACTGCAAACAGAAGGC | 4163 |
| Mef2a | GCAGGGACTCAGCATTGCTC | 4164 |
| Mef2b | GGGCCAGAGGAAGACCCAAG | 4165 |
| Mef2b | GCAGAGGGAAAGTCACTGTG | 4166 |
| Mef2b | GACAAAGCTGGAGCTGGCTG | 4167 |
| Mef2b | GCTGCACTAGAATGCTGTTG | 4168 |
| Mef2b | GTCCTCCCAGTTGCTCCAGT | 4169 |
| Mef2b | GAATGTCAGGGTCAGAGGIC | 4170 |
| Mef2b | GGAAGTAAGGCCCAGAAATG | 4171 |
| Mef2b | GTCATCGCCTCTGGCTATTC | 4172 |
| Mef2b | GCTCGCCTCTGGCTTTGCAG | 4173 |
| Mef2b | GTGAAGGGCTTTGGGATGTG | 4174 |
| Mef2c | GATACTGGGTGATGCCATTC | 4175 |
| Mef2c | GTTGGCTTCAGTCTTGGTCG | 4176 |
| Mef2c | GCTTGTAACTCTAAGAGACT | 4177 |
| Mef2c | GCTAAACCAGGTACATTTAA | 4178 |
| Mef2c | GATATCAGCAAGTGTTCAGC | 4179 |
| Mef2c | GAAAGCTAGAAGACAGAGGA | 4180 |
| Mef2c | GAGTTACAAGCTTTCTAATT | 4181 |
| Mef2c | GTGTGATGAGAGAAAGAAAC | 4182 |
| Mef2c | GAGAATGTTTCTCTACACTT | 4183 |
| Mef2c | GTCATGGCACTTAAACGATT | 4184 |
| Mef2d | GCTTCTGGATGTTTCCTGTG | 4185 |
| Mef2d | GGAAATGACAGAGTCTGGCG | 4186 |
| Mef2d | GAGAGTGAIGGACAAGCAGG | 4187 |
| Mef2d | GTCCCTGTTCTGGCTTCTTG | 4188 |
| Mef2d | GCCATTGGGTCCCAGCTTGT | 4189 |
| Mef2d | GCAGAATAGTCCTATTGAAC | 4190 |
| Mef2d | GTCACAGGTAGAGGGAGCAG | 4191 |
| Mef2d | GAGGCAAGGGAGGTAGTGGT | 4192 |
| Mef2d | GCCTAGCTTGCGAGATGGGA | 4193 |
| Mef2d | GAACTCTCCAGATGGCGCAG | 4194 |
| Meis1 | GTGTAAGACGCGACCTGTTA | 4195 |
| Meis1 | GCGTCGCCGCTGAAAGAGCT | 4196 |
| Meis1 | GTCAAAGCCAGAGCAAGAAG | 4197 |
| Meis1 | GAGCACCGGTGAAATTCCCA | 4198 |
| Meis1 | GTGAACATATGTCAACCTTC | 4199 |
| Meis1 | GAGGGCTGCAAGAGAGGAGG | 4200 |
| Meis1 | GCCGCATTGGTCTGGAGCTG | 4201 |
| Meis1 | GCCAGAGCAAGAAGAGGAGC | 4202 |
| Meis1 | GGGAATGCAAACTGCCATTC | 4203 |
| Meis1 | GCAATCTAAGCCACGAGAGC | 4204 |
| Meis2 | GAGTGAGTGTCAGTAGGTGT | 4205 |
| Meis2 | GAACTCGGAGCATAGTCCCT | 4206 |
| Meis2 | GCTCGTAACCTTCAGTTCGG | 4207 |
| Meis2 | GCAGGAGCCAAGAGGAGTGG | 4208 |
| Meis2 | GGGTCCTGGCCTCAATCTGG | 4209 |
| Meis2 | GTCAGTAGGTGTTGGCAGGT | 4210 |
| Meis2 | GCTCAAAGGGAGAGAAGGCA | 4211 |
| Meis2 | GAAAGCAGCGCCTCCTGCAA | 4212 |
| Meis2 | GATATAAATCCTCTCCTACA | 4213 |
| Meis2 | GTTGGCAGGTTGGCTGCAGC | 4214 |
| Meis3 | GTCTGAGCTAGGAAGACTTA | 4215 |
| Meis3 | GAGAGGCGGTGACTTCGGGA | 4216 |
| Meis3 | GGCACACTCAGGACAATAAG | 4217 |
| Meis3 | GTGGTGGTGACAGAAATAAG | 4218 |
| Meis3 | GACTGCACAGCCATGGCTAA | 4219 |
| Meis3 | GAGCCACCTCACTCAGTCTA | 4220 |
| Meis3 | GGCCTGAGAGGCTATGGAGG | 4221 |
| Meis3 | GAGCTGCTGTGCTTCCCTCA | 4222 |
| Meis3 | GGCTAGGCAGAGAGGACCTG | 4223 |
| Meis3 | GCAGTGAGGACCAAGAGGGA | 4224 |
| Meox1 | GTGAGATGGAAGGAGCCCAC | 4225 |
| Meox1 | GTTCCCTGTCAAGGCCCTGT | 4226 |
| Meox1 | GACATGGAGGCAGGAACCCA | 4227 |
| Meox1 | GCTGACAAATGGGTTGCTGT | 4228 |
| Meox1 | GAGGTGAGGTGTGCTGTCCC | 4229 |
| Meox1 | GTGATTAGCCCGGAGAGGTG | 4230 |
| Mecx1 | GGTAGAGAGTCTTTAAATCA | 4231 |
| Meox1 | GTCTACGCTATACCTATACC | 4232 |
| Meox1 | GAGACAAAGATGGATGGAGG | 4233 |
| Meox1 | GCTTGTGTATGTGCTGTGTT | 4234 |
| Meox2 | GTCCTGCAATTGCATGACTT | 4235 |
| Meox2 | GGAACCTATGGGACAGATTG | 4236 |
| Meox2 | GGGATGTCTGCAGTAGCCTA | 4237 |
| Meox2 | GGTTCCAGCGTAAACACATT | 4238 |
| Meox2 | GTTTGCATGTGGTCAGCGCT | 4239 |
| Meox2 | GCAGCAAGGCTTTGACGGTA | 4240 |
| Meox2 | GTCCTGCCAGCAATGGGAAC | 4241 |
| Meox2 | GGAGCTTCCACCACAGCTAG | 4242 |
| Meox2 | GATTTCATTTCTCAAAGGAT | 4243 |
| Meox2 | GAGACACTGTGTGCTGGCTT | 4244 |
| Mesp2 | GTATACAGCAAATTGGCTAA | 4245 |
| Mesp2 | GAATGACTTCCAGCCCTCCC | 4246 |
| Mesp2 | GAAGTGGAAATGGAAGGAGG | 4247 |
| Mesp2 | GAGAGCCCTTGGGCAGTGAC | 4248 |
| Mesp2 | GGCTGGGAAGTGGAAATGGA | 4249 |
| Mesp2 | GGAAAGGCCTGGAGGTGGGA | 4250 |
| Mesp2 | GAAGGGAAAGGCCTGGAGGT | 4251 |
| Mesp2 | GCAATTTCAGGATTAATCCA | 4252 |
| Mesp2 | GCATTGTTTCATTAGGGAGA | 4253 |
| Mesp2 | GAGGCACGGGATAGACATCC | 4254 |
| Mga | GAATGTCTGCCCTCACATTC | 4255 |
| Mga | GGAAACCAAGAATGTAAGGA | 4256 |
| Mga | GGAAAGGAGAGACAGGAGAG | 4257 |
| Mga | GAAGCTTCATAAGTTCTTTC | 4258 |
| Mga | GTTTGGCCTCCTGATGTTGG | 4259 |
| Mga | GAGTCTTCTTGGGAAAGGCC | 4260 |
| Mga | GCTCTAGAAATTGTGAGAAG | 4261 |
| Mir101a | GTTGGAAAGTACCAGAACAC | 4262 |
| Mir101a | GGCTTGAAACTTAACCTTCC | 4263 |
| Mir101a | GTTTGAGATGTGACTGACAT | 4264 |
| Mir101a | GGCAAATCACAGAATGTCCC | 4265 |
| Mir101a | GCAAATCACAGAATGTCCCA | 4266 |
| Mir101a | GCTATCTTTGCACTTTGGAG | 4267 |
| Mir101a | GCACGTTTATGGTTCTTGAT | 4268 |
| Mir101a | GTGTGAGGCTAGAAATCTTT | 4269 |
| Mir101a | GTGCATAGGTGTGAGATTGG | 4270 |
| Mir101b | GGAGTTCAGCAGGAGCCCAT | 4271 |
| Mir101b | GGGCTCTGCAAATGGGCAGA | 4272 |
| Mir101b | GAGCCCTCCCTTCCAAATTG | 4273 |
| Mir101b | GTTCTGCTGCTCATGACCCT | 4274 |
| Mir101b | GGAAGAGGTAAGACGCACTT | 4275 |
| Mir101b | GGTGTACTGGGAAGAAGGCA | 4276 |
| Mir101b | GAGCCGCTCTTGTCTTCAGC | 4277 |
| Mir101b | GTCCCTTTCTAGGAGACCAT | 4278 |
| Mir101b | GGTCAGATTTCCTGTTTGTA | 4279 |
| Mir101b | GACCTCAATTAATCTAACAC | 4280 |
| Mir106a | GGTCCAAGAGGATAGATATT | 4281 |
| Mir106a | GTCTGACTCTTAAGAGTAAG | 4282 |
| Mir106a | GAGAGTTAACTAAGGTGGGA | 4283 |
| Mir106a | GAAGGGCAAGGCTGAGGGAG | 4284 |
| Mir124a-2 | GTCTTCTTTGTGACCTGTAA | 4285 |
| Mir124a-2 | GGTGCTTTAGGATGGGCGGT | 4286 |
| Mir124a-2 | GATGGAAAGAAGAAGAATGA | 4287 |
| Mir124a-2 | GGCACAGGTTTGGTTCACTG | 4288 |
| Mir124a-2 | GTTAGATGGGTAAGGGCGCG | 4289 |
| Mir124a-2 | GAGATTGGAGAATGCGGTTC | 4290 |
| Mir124a-2 | GTGTTCTCGGAGGAAAGAGG | 4291 |
| Mir124a-2 | GGTAGAAAGCAGAGACAGTT | 4292 |
| Mir124a-2 | GACTGGAGAGGAGGGACAGG | 4293 |
| Mir124a-2 | GCCAGCCTGGACCTTGACTG | 4294 |
| Mir124a-3 | GCTGCCTGTGCGCTAAGAGA | 4295 |
| Mir124a-3 | GGGACAGTGCCAAGGAAGCC | 4296 |
| Mir124a-3 | GGGCCTTTGTTCCTGCAGAC | 4297 |
| Mir124a-3 | GAAGGGTTGTCCTGGGTGTG | 4298 |
| Mir124a-3 | GCGGTTCGAGAGTGTCCAAG | 4299 |
| Mir124a-3 | GCTCTCTTCTCTTACGCCTC | 4300 |
| Mir124a-3 | GACTGGCACCTGCAAAGGGA | 4301 |
| Mir124a-3 | GGGTTGGGCATAAGCAAAGG | 4302 |
| Mir124a-3 | GCTTCTGAGCCTCTCTCTCC | 4303 |
| Mir124a-3 | GCACTCACGCACTCCTGGTG | 4304 |
| Mir125a | GACCTCATTTCTGAGTTGGG | 4305 |
| Mir125a | GACAACTGACTTTGGTCTAG | 4306 |
| Mir125a | GGCCGGCAGTGTAGCTATGG | 4307 |
| Mir125a | GAGACCAGAAGTAGGGAGGG | 4308 |
| Mir125a | GCCTGGGATATGAAACCTTT | 4309 |
| Mir125a | GAGACTGGAAGATGGGAGGA | 4310 |
| Mir125a | GTCTGGGAGGTTGGGAAGGA | 4311 |
| Mir125a | GCTTCCCTGGATCTGTGGGA | 4312 |
| Mir125a | GCTCTGAGCCAGGTTGGTTG | 4313 |
| Mir125a | GTCCAGGTTGCTCTGAGGAC | 4314 |
| Mir125b-1 | GTTCAATAGGACAGAGAATG | 4315 |
| Mir125b-1 | GTGTTCAATAGGACAGAGAA | 4316 |
| Mir125b-1 | GTAGCTGTCTGTGAAGATGG | 4317 |
| Mir125b-1 | GAGCTAAAGGTGATTAGAGG | 4318 |
| Mir125b-1 | GTGTGTGGATGCCAAACAAT | 4319 |
| Mir125b-1 | GAGCTGAACCTACAGAGGTG | 4320 |
| Mir125b-1 | GGAAGGCTGTTGGGTGGGAG | 4321 |
| Mir125b-1 | GGGTTGGAGCACGTTCAAGA | 4322 |
| Mir125b-1 | GGCCATATCAGGACAAGGAG | 4323 |
| Mir133a-1 | GGGACAGCTGATCTAAGTGC | 4324 |
| Mir133a-1 | GTTAGTGATACATTGATGTA | 4325 |
| Mir133a-1 | GAGCAACTGCACTTGCTGAC | 4326 |
| Mir133a-1 | GAGTATGGAAGTCATCCTCC | 4327 |
| Mir133a-1 | GCAAATTATAAAGAAGAGGG | 4328 |
| Mir133a-1 | GTGAGTACATGTTAAACTCT | 4329 |
| Mir133a-1 | GCTAAAGGAAACTTTCCAGG | 4330 |
| Mir133b | GTTGGGTGCTTTAAAGTATG | 4331 |
| Mir133b | GGTTCTCTCTGTTACAGGCT | 4332 |
| Mir133b | GTACCTTGATGATTCGAGAC | 4333 |
| Mir133b | GAGTCTATCGAGGGAAACAG | 4334 |
| Mir133b | GAGATCAAGTGTAGGTAAGA | 4335 |
| Mir133b | GAGTCCATCTGGAAGAAGCC | 4336 |
| Mir133b | GACTTTAGTAGAGTCTATCG | 4337 |
| Mir133b | GCATGCCACCCTATTCTTCT | 3338 |
| Mir134 | GTAGGTCAGAAGTCCTCTGC | 4339 |
| Mir134 | GAATGATTCGGTGGGCTGCA | 4340 |
| Mir134 | GCTCTGAAAGGCTGCTAAGA | 4341 |
| Mir134 | GATGGCAACTTGCAGAAAGA | 4342 |
| Mir134 | GCTCTAGAAACACACTGGAG | 4343 |
| Mir134 | GAGCCACAGCTGCCTCACCA | 4344 |
| Mir134 | GTCTTCCTAAGAATGGATTG | 4345 |
| Mir141 | GGCTCGCAGGTGGATAGTAG | 4346 |
| Mir141 | GTGGAGGCCAAGTCGGCTCT | 4347 |
| Mir141 | GACGCCGATGACACTGGGAC | 4348 |
| Mir141 | GATCTGCCGCTTCTCTTGAG | 4349 |
| mir141 | GAGATCTGCCGCTTCTCTTG | 4350 |
| Mir141 | GGAGGAAGGAGCCGCTGGAA | 4351 |
| Mir141 | GGAAGCCTCTGCAGGGATCA | 4352 |
| Mir141 | GAAGAGTTGGCTCCCACCAT | 4353 |
| Mir141 | GCGGGTCTGGTGCCAGGTAA | 4354 |
| Mir141 | GGTGGGAGCCAACTCTTCCC | 4355 |
| Mir150 | GGTATGGTGATACCCATCTT | 4356 |
| Mir150 | GGAGTAGAGCCACTAAGCAG | 4357 |
| Mir150 | GGATCCAGGTGTTCTGAGAC | 4358 |
| Mir150 | GAAGACATTTCCACCGGGAG | 4359 |
| Mir150 | GTGTGGAACTTTCTTTGGGT | 4360 |
| Mir150 | GCAGAGGTTATGTATGGTTA | 4361 |
| Mir150 | GCGGGTGAGGCTTCTCAGCA | 4362 |
| Mir150 | GTTGCAGAGTCTGTGAGGGA | 4363 |
| Mir150 | GACCTGTTTCAAACGAAGCC | 4364 |
| Mir150 | GGCATATCACCATTTCTCTG | 4365 |
| Mir150 | GCTTGGAAATTTCCAAACCA | 4366 |
| Mir155 | GCCATATTATTGACCCATTA | 4367 |
| Mir155 | GCCACATAGTGAATGGGACC | 4368 |
| Mir155 | GCAGGTGCTGCAAACCAGGA | 4369 |
| Mir155 | GTGATATGCCACATAGTGAA | 4370 |
| Mir155 | GTTGCATATATTCTCCCTAA | 4371 |
| Mir15a | GTTATCCTAAGATGATGTTC | 4372 |
| Mir15a | GTGGTTTATATTCTGGCCTA | 4373 |
| Mir15a | GAACATCATCTTAGGATAAC | 4374 |
| Mir15a | GAAGCTTTGTCCIATGGATT | 4375 |
| Mir15a | GACACTCAAAGGACAGTGTC | 4376 |
| Mir15a | GCTGGCACACTTGAAAGCAA | 4377 |
| Mir15a | GGAAACAAATAGAGTTGAAG | 4378 |
| Mir15a | GCGTGCTGGAGGAAGTGCTT | 4379 |
| Mir16-2 | GCATATGTGTGTAAAGAGTC | 4380 |
| Mir16-2 | GTTAAGGGAGAGGCAAAGAG | 4381 |
| Mir16-2 | GAGGTCTTGTTCGCCTTCCT | 4382 |
| Mir16-2 | GGCTGAAATTTGTGTTTGCT | 4383 |
| Mir16-2 | GAGGCTCTAGGTTAAGGGAG | 4384 |
| Mir16-2 | GCTGGATAACAGAAGTTTAG | 4385 |
| Mir16-2 | GCTCCTCACCTGGAGGCTCT | 4386 |
| Mir16-2 | GCTATCTCTGTAGGCGGTTC | 4387 |
| Mir181a-1 | GCATTGATCTGACAAATGAG | 4388 |
| Mir181a-1 | GATTCCAGAATGACTGGAGT | 4389 |
| Mir181a-1 | GCAAAGCACCGCAATGTGAG | 4390 |
| Mir181a-1 | GATTACAGGACAAGTGTCTC | 4391 |
| Mir181a-1 | GAATTTCAGGCAGTAGGCAT | 4392 |
| Mir181a-1 | GTTACAGGCTGTTAAAGACA | 4393 |
| Mir181a-1 | GTAAGAGAATAACTTCAGGA | 4394 |
| Mir181a-1 | GATCTGACAAATGAGAGGGA | 4395 |
| Mir181b-1 | GGTCCTTAGAATATGAGAGC | 4396 |
| Mir181b-2 | GCAACCAAGCCAGCCTTAAG | 4397 |
| Mir181b-2 | GAATCCCAAGGTACAGTCAA | 4398 |
| Mir181b-2 | GAACTCTGGTGTTCAAGTTC | 4399 |
| Mir181b-2 | GAGCATCACTAGCACTTCTG | 4400 |
| Mir181b-2 | GTGTCATTCTAGTCAGAAAT | 4401 |
| Mir181b-2 | GTGCTAATTTAAGGAATTCT | 4402 |
| Mir181b-2 | GCAACATATCCAACCAATAC | 4403 |
| Mir192 | GAGTTGCTGTTACAGAGGGT | 4404 |
| Mir192 | GGAGTTGCTGTTACAGAGGG | 4405 |
| Mir194-2 | GAAGGCTTGGCTTAGGGCTC | 4406 |
| Mir194-2 | GGAAGCCTCTAGAGTATGCT | 4407 |
| Mir194-2 | GCCAACTGGCCGAGAGAGTG | 4408 |
| Mir194-2 | GATCAAGGCTTAGACAGAGT | 4409 |
| Mir194-2 | GGCAGCTCTGCTGCTTCTCT | 4410 |
| Mir194-2 | GGGAGCCTTCAGCAGCCTTC | 4411 |
| Mir194-2 | GATGGCTTGGCAGGAAGGCT | 4412 |
| Mir194-2 | GGGTCCAGGAAGTACCAGAC | 4413 |
| Mir194-2 | GGGATAGATGCCATGTGGGT | 4414 |
| Mir194-2 | GAGAAGCAGCAGAGCTGCCA | 4415 |
| Mir196a-2 | GAGAGCAAACTGCAATCTTG | 4416 |
| Mir196a-2 | GATAGTCTCCCGTTAGTTTC | 4417 |
| Mir196a-2 | GAGGGTTTAGTCTAGACACT | 4418 |
| Mir196a-2 | GGAATAAACTTAACTGCCGG | 4419 |
| Mir196a-2 | GGCTGACAGCAAAGAGCGGA | 4420 |
| Mir196a-2 | GGGAAAGACAGAGAGAGGGA | 4421 |
| Mir196a-2 | GTCAAATGCACCCGATTAGA | 4422 |
| Mir196a-2 | GGAGCAGGACAACTTGGAGG | 4423 |
| Mir196a-2 | GCGGCAGCAAGAGAAGGAGG | 4424 |
| Mir196a-2 | GAACCGAGAGAATCGGATCC | 4425 |
| Mir196b | GGGCTGGGTTTGCTGCCTCT | 4426 |
| Mir196b | GCGTGGGTTCTTCTGGGACC | 4427 |
| Mir196b | GGCGCCTAGGAGGGAGAAGA | 4428 |
| Mir196b | GGTGTCTGGCCTGAGGTCAA | 4429 |
| Mir196b | GAACCCACGCCCGAAATCCG | 4430 |
| Mir196b | GGAAACTCAAAGGTGAATGA | 4431 |
| Mir196b | GTATGGAAGCATGGACATTC | 4432 |
| Mir196b | GAGGACCGGGTGTGGATTTG | 4433 |
| Mir199a-2 | GCAGGTACAAATAAGTTGTT | 4434 |
| Mir199a-2 | GGCTTCCTACAATAGCGTGG | 4435 |
| Mir199a-2 | GGCACATTTGCAGCAGACTA | 4436 |
| Mir199a-2 | GGCCTCCTTCTCCTTCTTTA | 4437 |
| Mir199a-2 | GGGTGACATCATCCCATATA | 4438 |
| Mir199a-2 | GATTCTAGCGGTCTCTCCAG | 4439 |
| Mir199a-2 | GGGCTGGAGAGTCCATATAT | 4440 |
| Mir199a-2 | GGACTAGGCATAGAAAGGGA | 4441 |
| Mir199a-2 | GGACTATTTGAGAGTGGTTA | 4442 |
| Mir199a-2 | GGGAATGATGACCAAGAGGA | 4443 |
| Mir1a-1 | GCTCCCATTGCGTCCGCACT | 4444 |
| Mir1a-1 | GTGTCTCCAGCTCTTTCTGT | 4445 |
| Mir1a-1 | GTAAAGACTGGAAGCAGACA | 4446 |
| Mir1a-1 | GTAAGTTTAGCCACAATCTC | 4447 |
| Mir1a-1 | GGCACTGAGACCTTCTCTCG | 4448 |
| Mir1a-1 | GGACTGATGGATCAGGAACT | 4449 |
| Mir1a-1 | GGATGTGACTTCCCTCTGTT | 4450 |
| Mir1a-1 | GTCGTAAGGAACCGCTCCCA | 4451 |
| Mir1a-1 | GACACCCACTGCAGGAGAGG | 4452 |
| Mir1a-1 | GAGTTCTCAGGGAGCCTAAG | 4453 |
| Mir200a | GAGGAAGGACTTAGCACCCA | 4454 |
| Mir200a | GACGGACTTGGGATGAGGAG | 4455 |
| Mir200a | GCATCTACTAGGCTTAGTTT | 4456 |
| Mir200a | GATCAAGGCACTCTGGAAAG | 4457 |
| Mir200a | GTCCCAAGTATCCTTGGGAC | 4458 |
| Mir200a | GGTCTGCTTTGTCCAAAGCA | 4459 |
| Mir200a | GCGGCTCCATTGCTGCATGC | 4460 |
| Mir200a | GCGGCCTCCATATCCAACTT | 4461 |
| Mir200a | GGATACTGGGATGAGGGACC | 4462 |
| Mir200a | GATCCGAGGAAATCAGTACA | 4463 |
| Mir200b | GTTGGAACTGCGTGTCTTCA | 4464 |
| Mir200b | GTCATCTTCAACTCCCTGCT | 4465 |
| Mir200b | GCCTGCCTCCCAGCTCTTTC | 4466 |
| Mir206 | GCGTCACTAACTGTGAGGCC | 4467 |
| Mir206 | GTCTGACTGATCACCCTGGA | 4468 |
| Mir206 | GGCAGCTGTTGAGCCATTCA | 4469 |
| Mir206 | GATCTCAGACTGAAGTGTAT | 4470 |
| Mir206 | GCCTAACAGGCAGAGCTTGT | 4471 |
| Mir206 | GACTAGTATGCTAGTATGCC | 4472 |
| Mir206 | GAACAGCCTTGGATCAGTCC | 4473 |
| Mir206 | GGCCAAACTTCCTGCACATT | 4474 |
| Mir206 | GACCAATCCACCAAATGTGC | 4475 |
| Mir21 | GCAGAGACGGACCTATGCCG | 4476 |
| Mir21 | GTTAGAGCCCTCCCAGTGTA | 4477 |
| Mir21 | GTTTCCTCGGTTCAACACTA | 4478 |
| Mir21 | GAGATCTAAGCGGGACTATG | 4479 |
| Mir21 | GGCCCTGTGAAGGTATCAGA | 4480 |
| Mir21 | GGGACAGTCAGAGAGAGGGA | 4481 |
| Mir21 | GAGCCCTCCCAGTGTAAGGC | 4482 |
| Mir21 | GTTCTGCTTTCTTTCCTACA | 4483 |
| Mir21 | GCAGGAGGGATCCTCACCTG | 4484 |
| Mir21 | GCCTGAGAGAGCTACCTCCA | 4485 |
| Mir218-2 | GACTAAGAGAAGGAAGGAAA | 4486 |
| Mir218-2 | GGTCCTGTAAACACCAAGGC | 4487 |
| Mir218-2 | GTACTAATCACGCTCAGTGG | 4488 |
| Mir218-2 | GGATCCTTTGGGTACAACAC | 4489 |
| Mir218-2 | GTGAGGGCCTTGGTATGAGT | 4490 |
| Mir218-2 | GGACACAACCTCTGATGGGA | 4491 |
| Mir218-2 | GAAGCCAGACGCCCTACCCA | 4492 |
| Mir218-2 | GGAGAAGCTGAAGCCAGAGC | 4493 |
| Mir218-2 | GCTAGGTCACTGCCATGGTG | 4494 |
| Mir23b | GACATTATCGCTTGCCATGG | 4495 |
| Mir23b | GGGCTAGAGCCACTTTGAAT | 4496 |
| Mir23b | GTCTGCAGGAGGCAGTGAAG | 4497 |
| Mir23b | GGTTCTCTGACCTGTAGAGT | 4498 |
| Mir23b | GACAATGGAGACAGAGTAGA | 4499 |
| Mir23b | GAGGGCTGCCAAACGGTCTT | 4500 |
| Mir23b | GCAGGTGTGGTGTGTAGGGA | 4501 |
| Mir23b | GACAGAGTCAAAGTGAGGGC | 4502 |
| Mir23b | GGAGAACAGGGTGTGTCCCA | 4503 |
| Mir6a-2 | GGCCTAAGGAACACTTGTGC | 4504 |
| Mir6a-2 | GATGTCTGCATCACTGTCTC | 4505 |
| Mir6a-2 | GGTCTCTCACCAATGCCTCG | 4506 |
| Mir6a-2 | GATTGGGCTTACTTCTTGTT | 4507 |
| Mir6a-2 | GGCAGTTTCCCTTTGAGGCA | 4508 |
| Mir6a-2 | GTGTTGGCTAGAGGGAAGTG | 4509 |
| Mir6a-2 | GATGTGGGCTAGGAGGGACT | 4510 |
| Mir6a-2 | GATCGGACTGTGTGAGACAA | 4511 |
| Mir6a-2 | GCTGGCTAAGAACTGCTCAG | 4512 |
| Mir6a-2 | GGGTATCTGTGACTCCAGGG | 4513 |
| Mir375 | GCCATTGGGAGGTGAGCAGC | 4514 |
| Mir375 | GGATGCACAAGAAGCTATGT | 4515 |
| Mir375 | GTTCTTAGTTTGGCCAGTGG | 4516 |
| Mir375 | GGGCAAATATTGACTCATGG | 4517 |
| Mir375 | GCTGACACCAGCAAACAGTC | 4518 |
| Mir375 | GATGTTCTGCCTTCGCTAGG | 4519 |
| Mir375 | GAGTGCTCTGAGTCCTGGCT | 4520 |
| Mir375 | GTCAGCATGCACAGGTCAGG | 4521 |
| Mir375 | GGTGGTAGGGCAATGATGCG | 4522 |
| Mir375 | GTGGGAAGATTCTATCTCCA | 4523 |
| Mir7b | GAAGGCCAACTGGACTGTTT | 4524 |
| Mir7b | GGACTCTGAGTCCTTGAACT | 4525 |
| Mir7b | GGAGGGTAAGTCAGTGAGTG | 4526 |
| Mir7b | GTGAGAGAGACTGTGTTAGA | 4527 |
| Mir7b | GCACTTGAGGGTGTTGAACC | 4528 |
| Mir7b | GGTGTTGAACCTGGCGGAGG | 4529 |
| Mir7b | GGTTCATTCTATACACCCTA | 4530 |
| Mir7b | GAGGGACTCGGAGCAGAGTT | 4531 |
| Mir92-2 | GGAGGGAAACCAAGGTAGGT | 4532 |
| Mir92-2 | GGCCTCTGATTAAATCACCA | 4533 |
| Mir92-2 | GTAATGTGTCTCTTGTGTTA | 4534 |
| Mir92-2 | GAGCGGGTCCTGTGTGTCAC | 4535 |
| Mir92-2 | GTGGTGCTGCGCGGACACTT | 4536 |
| Mir92-2 | GCTCTCCTAGCTGGTGGAGG | 4537 |
| Mir92-2 | GCACTGTTAGCACTTTGACA | 4538 |
| Mir92-2 | GATGGAATGTTTGTGTTGAT | 4539 |
| Mir92-2 | GAGCTTTCTCTGGAGGGCTG | 4540 |
| Mir92-2 | GTTGTGTAGAAGAACAAGCT | 4541 |
| Mirlet7a-2 | GGAACATACCATGGTACGGC | 4542 |
| Mirlet7a-2 | GACCCATACAACTCTGCAAG | 4543 |
| Mirlet7a-2 | GAAGACTGTGCAAGAGACTA | 4544 |
| Mirlet7a-2 | GAGGCCAGGTTGAAAGATTG | 4545 |
| Mirlet7a-2 | GGTTTGAGATTGCTCCGTGG | 4546 |
| Mirlet7a-2 | GTTGTATTGTAGATAACTGC | 4547 |
| Mirlet7a-2 | GTTTGAGATTGCTCCGTGGT | 4548 |
| Mirlet7a-2 | GGTCAAAGATTCAAAGAAGC | 4549 |
| Mirlet7b | GGAATAGCTAGAGACCACAT | 4550 |
| Mirlet7b | GTCTGAGGCCTGAAAGAAGC | 4551 |
| Mirlet7b | GCCCAGGTGAGAAGGCTGAG | 4552 |
| Mirlet7b | GGTAAAGACATCTAAGCTGA | 4553 |
| Mirlet7b | GCTAGTCGTTAGGGACAGAC | 4554 |
| Mirlet7b | GCTGCCTGGCTTCCTAGGTC | 4555 |
| Mirlet7b | GGCCCAGGTGAGAAGGCTGA | 4556 |
| Mirlet7b | GCCTAGAGAAAGGCCAGATG | 4557 |
| Mirlet7b | GCAGCAAGGCAGAAGAGGCG | 4558 |
| Mirlet7b | GAGGCGTGACAGTAGACGCT | 4559 |
| Mirlet7i | GGTGTTGCACTGCCTTATCT | 4560 |
| Mirlet7i | GGCGCTGTAAAGATGGCGGC | 4561 |
| Mirlet7i | GCAAGGATGCAGAGAGGAGA | 4562 |
| Mirlet7i | GTATGTATGAAACGTGTAGG | 4563 |
| Mirlet7i | GGACTGGGTGGGTGTGAGGT | 4564 |
| Mirlet7i | GGCAGTGCAACACCGGAACC | 4565 |
| Mirlet7i | GAGAGTAGGGAAACCAGCCG | 4566 |
| Mirlet7i | GGGCGCTGTAAAGATGGCGG | 4567 |
| Mitf | GAAGTCAGCAAATGGTGGTG | 4568 |
| Mitf | GACACTCCTGAAAGTTGGGC | 4569 |
| Mitf | GACACACTGGAAGTGGAATC | 4570 |
| Mitf | GCCATAAGCAGTCAGAATAT | 4571 |
| Mitf | GTGGGATGGACAGATGGAAA | 4572 |
| Mitf | GGGCTGTGTTGGGAAGAAGA | 4573 |
| Mitf | GAATTGTTACAGGGAGAACC | 4574 |
| Mitf | GTCTGGTCTGGACACCTCTT | 4575 |
| Mitf | GTAAGCTGTCTGTTGAGACT | 4576 |
| Mitf | GCTGACCTCAGCCTGGTAAA | 4577 |
| Mixl1 | GCGCCTTTGATGGTGACAGG | 4578 |
| Mixl1 | GGGAGGCGCGAACTTGAGTC | 4579 |
| Mixl1 | GAATTCTTCAACCTGCTACG | 4580 |
| Mixl1 | GTAAGGTCTAGCACATAGCA | 4581 |
| Mixl1 | GCTTGACCTGTCCACCAGCT | 4582 |
| Mixl1 | GCTAGGCTGTTTAACCAACC | 4583 |
| Mixl1 | GAAGAAGAAAGAAAGGGAGA | 4584 |
| Mixl1 | GGGCAGACAGAAGGTGGCAG | 4585 |
| Mixl1 | GGATTGGTGGTTGGACTGGC | 4586 |
| Mkl1 | GAACCACGAGTGTACGCTAT | 4587 |
| Mkl1 | GGGAAGGATGAGACTGCCCT | 4588 |
| Mkl1 | GGCAAATAGCAGTTGGATTC | 4589 |
| Mkl1 | GACCTCCTCCCACCTCTTGG | 4590 |
| Mkl1 | GTTAGGGCTAGCCCGATTTA | 4591 |
| Mkl1 | GCTCTTAAACACCGTGTTCT | 4592 |
| Mkl1 | GGCAGAGAGAGAGGCGTCAT | 4593 |
| Mkl1 | GTGCTTCACCAGAAAGAGTC | 4594 |
| Mkl1 | GGCATTTATTGTGTCCTTTC | 4595 |
| Mkl1 | GAAGTCTGGAACTGGCGGAG | 4596 |
| Mlx | GCGGCTTAACTGTCCCACTT | 4597 |
| Mlx | GCAATGAGGACACAGCTAAT | 4598 |
| Mlx | GATGACACACGGGTCAGGAA | 4599 |
| Mlx | GTTCAGGAACTTGTCTGTGG | 4600 |
| Mlx | GCATCTGACTGAGTTCCTGG | 4601 |
| Mlx | GGCCCATAGGGATCCAGCAG | 4602 |
| Mlx | GACTGAGCCTCGCCTCTTCC | 4603 |
| Mlx | GAACAGGTACTAGCCAGAGA | 4604 |
| Mlx | GGTCCAGATACCTCAGTCTC | 4605 |
| Mlx | GAGGCTGAAGCAGGTTTCCC | 4606 |
| Mlxip | GGACTCAGTTCCGGGTATGG | 4607 |
| Mlxip | GCACTCCACGTGGTGGGTAG | 4608 |
| Mlxip | GCTGAAGTTGTTGGGTCTGG | 4609 |
| Mlxip | GTTTAAGAGCGGTGATGCCC | 4610 |
| Mlxip | GTGGCTGAAGTTGTTGGGTC | 4611 |
| Mlxip | GGCACTCCACGTGGTGGGTA | 4612 |
| Mlxip | GGAGCTTGGGAATAGCCCTG | 4613 |
| Mlxip | GGAGAAAGCTGGCCTAATGT | 4614 |
| Mlxip | GAATTGCAGTAAAGACAACT | 4615 |
| Mlxip | GCCCAGAAGCCAAATTCCAA | 4616 |
| Mlxipl | GTTAGACTGTAGAGAGGCAC | 4617 |
| Mlxipl | GGCTGTGAACTCTGGGCATC | 4618 |
| Mlxipl | GGACAATCATAAGAGCGCCT | 4619 |
| Mlxipl | GGCCTCTCTTTCCCACTAGA | 4620 |
| Mlxipl | GGAGAGCAACCGATGGTTGG | 4621 |
| Mlxipl | GGCATCGGGTACTAGAGGGC | 4622 |
| Mlxipl | GCTAACCTTTCCACTGGGAC | 4623 |
| Mlxipl | GAACTTTGCTGTAGAGGCAT | 4624 |
| Mlxipl | GACATAGCTAACCTTTCCAC | 4625 |
| Mnt | GGAAATGGAGACATGCCAGT | 4526 |
| Mnt | GAGGAATAGCACAAGACAGA | 4527 |
| Mnt | GCCTGGTGATCTAGCCTAAT | 4628 |
| Mnt | GGGAATTGCGACAGACCGGA | 4629 |
| Mnt | GTCTGGGTCAGGAGGGCAAC | 4630 |
| Mnt | GTATGTTTATAGGTAAGACC | 4531 |
| Mnt | GCACTGGAGCTGTAAGTGTG | 4632 |
| Mnt | GAAGAGGGAAATGAATGGGA | 4633 |
| Mnt | GGGAGGGTAATGTAAAGCAG | 4634 |
| Mnt | GGAAGGGTGAGACACCTACA | 4635 |
| Msc | GGCTTTGTTAACAAACAGAC | 4636 |
| Msc | GAGTAATGAACTTGAATGAC | 4637 |
| Msc | GATTGCTTAAACTTGACTGT | 4638 |
| Msc | GGTGCAGGCAGAAAGATGGA | 4639 |
| Msc | GTAGTGAGCAGCTGCAGCTT | 4640 |
| Msc | GAAGTATCATAGCAGGTGGC | 4641 |
| Msc | GATGTGTGTTTGCTTATCCA | 4642 |
| Msc | GGCAGAAAGATGGAAGGCAG | 4643 |
| Msc | GGGCTGCTTGGTAGTCCTTT | 4644 |
| Msx1 | GTTATTTGTCAGAGTAGCAA | 4645 |
| Msx1 | GCCGATTTACACTCTGCGCT | 4646 |
| Msx1 | GGGTAATTATCCGAGCACGG | 4647 |
| Msx1 | GGAGGTATATCTTTGGTGCA | 4648 |
| Msx1 | GCAACTGTGTAGACAACTTC | 4649 |
| Msx1 | GATGCCCACCTGACTTAGCT | 4650 |
| Msx1 | GAGCCTCACATCTGCCCACA | 4651 |
| Msx1 | GGGCTGCCGTGGCCATTTAG | 4652 |
| Msx1 | GAGGTGATTGGCGGCTCACC | 4653 |
| Msx1 | GAGCAAAGAGGCCTAGCCTC | 4654 |
| Msx2 | GAGAAGGCTGTAGACGGGCC | 4655 |
| Msx2 | GCCAGAGCTTGGTACTCTGG | 4656 |
| Msx2 | GCACCAGAAACACTTTAAAG | 4657 |
| Msx2 | GAATGTTGGAAATCTGCGGA | 4658 |
| Msx2 | GCCTAGAGAGGAGACTCAAG | 4659 |
| Msx2 | GACTGTATCTCTGCCTAACC | 4660 |
| Msx2 | GGTGCTGGAGGGAGTATTTA | 4661 |
| Msx2 | GACACTGAAAGGGAAACGGT | 4662 |
| Msx2 | GTTGGAGAGGCGCCTGGCAA | 4663 |
| Msx2 | GAGGCGCCTGGCAAAGGGAT | 4664 |
| Msx3 | GATGAGTGTTTACCAAGGAG | 4665 |
| Msx3 | GGGCTAGAAAGACGCGTCCT | 4666 |
| Msx3 | GTCGACAGCAATGACTCATT | 4667 |
| Msx3 | GATGCAGTCTTTCCTTGACC | 4668 |
| Msx3 | GTCATCAGTTTGTGGACAAT | 4669 |
| Msx3 | GTCCATTCCTCCACTCCAGA | 4670 |
| Msx3 | GCCTCAGCCTTCTGGAGTGG | 4671 |
| Msx3 | GATCTCTTGAGGTCGAGTTG | 4672 |
| Msx3 | GGGCTACAGGGTAGGAGTGG | 4673 |
| Msx3 | GGGCTGAGTCTTCAATGGTG | 4674 |
| Mtf1 | GCTCAGGTAGAAGAAACAGG | 4675 |
| Mtf1 | GCCACAAAGGACTAGCTGCC | 4676 |
| Mtf1 | GAACTGGIGAATAAACTCTT | 4677 |
| Mtf1 | GCCTTGGAGTTGAGCAGAAA | 4678 |
| Mtf1 | GATGCTCAGTACGGGATATG | 4679 |
| Mtf1 | GCTTGGACAGTGGAAGCATC | 4680 |
| Mtf1 | GTTCTTGAGCTGAAACAGGT | 4681 |
| Mtf1 | GCAAGGGAGAAGAGAAGGGA | 4682 |
| Mtf1 | GGTTCCAACCTTCCTTAAGG | 4683 |
| Mtf1 | GGACTAACTGAAGTCCCTGA | 4684 |
| Mxd1 | GGCAATGACCTCCACCCAGC | 4685 |
| Mxd1 | GTTATAGGAGAGGACTGAGC | 4686 |
| Mxd1 | GTGACGTCATCGTAGCCGGG | 4687 |
| Mxd1 | GACAGTGGGCAACAGGTCGG | 4688 |
| Mxd1 | GGGATGGAAGGGATGGCCTC | 4689 |
| Mxd1 | GCGGTTTGAATTTAGTTCTG | 4690 |
| Mxd1 | GAGGTGACGTCATCGTAGCC | 4691 |
| Mxd1 | GAGTATCTAGCGCCATCTAC | 4692 |
| Mxd3 | GGTAGCCATACCTATGAGTC | 4693 |
| Mxd3 | GAGCAATCTGTAGCAGGAGA | 4694 |
| Mxd3 | GGCTGTTACCTATACCTCCT | 4695 |
| Mxd3 | GCTCCTCCTTCTCTTCCAAG | 4696 |
| Mxd3 | GCATCAGTTCTACTGCAGCA | 4697 |
| Mxd3 | GTAACAGTTTGTAGACTGAA | 4698 |
| Mxd3 | GAAGGACGGGAGAGCTAGGA | 4699 |
| Mxd3 | GTCCTGTCTGCCTCTGCTAC | 4700 |
| Mxd3 | GGCCCTATCTACATATTCAT | 4701 |
| Mxd4 | GCGTTCGGCCAGTCCCTATT | 4702 |
| Mxd4 | GCAGAATGAGCTGGCTTCCC | 4703 |
| Mxd4 | GACAGCAAGCCTGCTGCTCA | 4704 |
| Mxd4 | GATTGTGGGCTTGGTCAGAG | 4705 |
| Mxd4 | GTAGGCATTGCACGCCGATT | 4706 |
| Mxd4 | GTGCCATCTTCCCTCACAAA | 4707 |
| Mxd4 | GATCCGTGAGTGTCTGTTTG | 4708 |
| Mxd4 | GACATGTGTGGCTCACACCC | 4709 |
| Mxd4 | GTCTGGGCTCTGTCTATACA | 4710 |
| Mxd4 | GTTTGCGGTGCTTGGTCTGA | 4711 |
| Mxi1 | GGTGTGTCCACGCATACATG | 4712 |
| Mxi1 | GGGCGGGACTACATTTCCCA | 4713 |
| Mxi1 | GCTAGGATTTGCGGAGAGGC | 4714 |
| Mxi1 | GTCTCCAGGCTACCCTGTCC | 4715 |
| Mxi1 | GGAGGGAAGAAGAGGTTCCT | 4716 |
| Mxi1 | GGAAAGACTACATCTCCCGG | 4717 |
| Mxi1 | GACTTTATTTACTGAGAGGG | 4718 |
| Mxi1 | GTCTCTGGGCTGGTGAGGAC | 4719 |
| Mxi1 | GATCATCCGCACCCGCTCCA | 4720 |
| Mxi1 | GAATGAACCTACAGGACGGA | 4721 |
| Myb | GGATTCAAGAGGCTCAGGAA | 4722 |
| Myb | GTCCAGCAAGTGTTTGACGC | 4723 |
| Myb | GTGAGTGTCCCAAGTGCTTT | 4724 |
| Myb | GGAAGAGAATGCTTCTGTAA | 4725 |
| Myb | GGATGCAATAGATGCAACTT | 4726 |
| Myb | GGCGTGTGTCTAAGTGAGGG | 4727 |
| Myb | GTGGTAGGCACCTCCTAGGG | 4728 |
| Myb | GCTCCCGGGTGTGTTGAAGT | 4729 |
| Myb | GTTCAAGACTTGTGCTGACT | 4730 |
| Mybl1 | GCCGTTTGAATCTGCGCACG | 4731 |
| Mybl1 | GGCCAGTTTCCTTGTCCTTT | 4732 |
| Mybl1 | GCTGTGAGTCTCGCCACTTA | 4733 |
| Mybl1 | GGTGACAGGACACGGAACGC | 4734 |
| Mybl1 | GAGTAACTGAAATCTTGCAT | 4735 |
| Mybl1 | GCAACTTCTCAACAGTTACA | 4736 |
| Mybl1 | GAAGAACACTTGAAGTTCTC | 4737 |
| Myc | GACGAACGAATGAGTTATCT | 4738 |
| Myc | GGATACCGCGGATCCCAAGT | 4739 |
| Myc | GAGCTCCTCGAGCTGTTTGA | 4740 |
| Myc | GAATTGCCAACCCAGATCTG | 4741 |
| Myc | GATGACCGGAAGCTTGTCTT | 4742 |
| Myc | GAAGTCCGAACCGGAGGTGC | 4743 |
| Myc | GCCCGAACAACCGTACAGAA | 4744 |
| Myc | GACGAGCGTCACTGATAGTA | 4745 |
| Myc | GCCTTGGCTTCAGAGGCTGA | 4746 |
| Mycl | GACTGTTCGAGAGGCTCCCG | 4747 |
| Mycl | GTCTAACTACTCAGAACTAC | 4748 |
| Mycl | GCTAATGGTTACTGAAGCAA | 4749 |
| Mycl | GTGCGTCCCACCCATGACAG | 4750 |
| Mycl | GAACACTATCAAGATCTCGC | 4751 |
| Mycl | GGGAAGTAGACTAGCAGGGT | 4752 |
| Mycl | GGACGCACCTGAACCTGGTG | 4753 |
| Mycl | GACCAGTTCAGCCAGGAGGT | 4754 |
| Mycl | GAGGATGAATTCTGGGAGGC | 4755 |
| Mycs | GACCTGGTGGGTGGATTCAA | 4756 |
| Mycs | GCTCTCAAGAGCATCTTCCC | 4757 |
| Mycs | GGCACAGGACATGATGCTCC | 4758 |
| Mycs | GCACAGGACATGATGCTCCT | 4759 |
| Mycs | GTGGATTCAAAGGAGGGTGG | 4760 |
| Myef2 | GGTTAAGGGAATGATCACTT | 4761 |
| Myef2 | GCTAGTAATAGTAACCAGAT | 4762 |
| Myef2 | GGAGAATTTAATTCCCTCAC | 4763 |
| Myef2 | GCCTTTGGATGAGAGGACTA | 4764 |
| Myef2 | GCTTATGATAATCTAGAACT | 4765 |
| Myef2 | GTGAATTCATAAAGAGCTAA | 4766 |
| Myef2 | GGACACTAGAGCTCTGCTGG | 4767 |
| Myef2 | GAGTTCAATGTTGCCTTCTG | 4768 |
| Myef2 | GAACTGAAATGCCTCAGCCG | 4769 |
| Myef2 | GGGACAATTTAGCTGGAAGA | 4770 |
| Myf5 | GAACAATAAATCAACCGTGC | 4771 |
| Myf5 | GGAAGGATGGAAGCTCGGAG | 4772 |
| Myf5 | GGGAAGGATGGAAGCTCGGA | 4773 |
| Myf5 | GGAGGTTGGTCCCTGTAGCT | 4774 |
| Myf5 | GTCCCAAAGGGCCCTCCACA | 4775 |
| Myf5 | GACACGTGTGCTGGGAAGGA | 4776 |
| Myf5 | GTTTGTGTACTGGTAACAGT | 4777 |
| Myf5 | GGTTAGGGCTGTCTTTGGTA | 4778 |
| Myf6 | GAATCCTAAGCAACCAACTT | 4779 |
| Myf6 | GAATTCAGTTGAACTCTGGA | 4780 |
| My46 | GGGCTGGAATTGGAGTGTGT | 4781 |
| Myf6 | GTGAACTAATGTTTACTGCA | 4782 |
| Myf6 | GGTATGCAACCGCATTAACT | 4783 |
| Myf6 | GAATTATGAGAAGACAGAGC | 4784 |
| Myf6 | GATGACTCTCTGTCTTGATA | 4785 |
| Myf6 | GCACTAATTAAATGCCATCT | 4786 |
| Myf6 | GTGTTAACTATAAGCTGTTT | 4787 |
| Myocd | GGTACATCTCCAGAACGCGC | 4788 |
| Myocd | GATGGATGGGTAGGGAGGCA | 4789 |
| Myocd | GCTTACTGCAGGGCTCTGGA | 4790 |
| Myocd | GCAGCTGACTTCTGCCCTCC | 4791 |
| Myocd | GGAGTGTATCTGCTTGTCCT | 4792 |
| Myocd | GTCTTTCTGACCCAGAGGGA | 4793 |
| Myocd | GGTCCCTTTCCCACTATGAA | 4794 |
| Myocd | GACTAATCTCTGCCCTGATC | 4795 |
| Myocd | GTTTCACAGAGTTTCCTCCA | 4796 |
| Myocd | GGCAGCCTATGACATCAGCC | 4797 |
| Myod1 | GCTGGTTATGCTATGCAAGC | 4798 |
| Myod1 | GCAAAGCCAGAGAAGGTTGC | 4799 |
| Myod1 | GCATGAACATCCCAGGGTTG | 4800 |
| Myod1 | GAGCTGGAAAGGGAGGCTGG | 4801 |
| Myod1 | GGTGCTCATGGCCACTCAGA | 4802 |
| Myod1 | GGAGCCATTAAGAAGAATGG | 4803 |
| Myod1 | GGACAGAAAGGTGATCCATT | 4804 |
| Myod1 | GGTCTCCAGAGTGGAGTCCG | 4805 |
| Myod1 | GGATGTGGAAATGTCAGTGG | 4806 |
| Myod1 | GAGATCTGGCAGAGGGCTCT | 4807 |
| Myog | GCTGGGTGAAAGGTGGCCAG | 4808 |
| Myog | GCTGGTGGACAGGGCAGGAA | 4809 |
| Myog | GCGTTGGCTATATTTATCTC | 4810 |
| Myog | GTCCAAGGCAGCTGGTGGAC | 4811 |
| Myog | GCAGGAAGGGAACAAGAAAG | 4812 |
| Myog | GAAAGGAGCAGATGAGACGG | 4813 |
| Myog | GATTGAAGTAAGAGAACACA | 4814 |
| Myog | GCTTCTTCACTTTGAGGAGG | 4815 |
| Myog | GGCAAAGACAGAAACCCAGA | 4816 |
| Myog | GAGAGAGTAGGCAGGAGGCC | 4817 |
| Mzf1 | GTTGTATCTGACCTGAATTC | 4818 |
| Mzf1 | GCAAACCAGGAAGTCTCTTA | 4819 |
| Mzf1 | GTGAGACATCGAAACTCTAG | 4820 |
| Mzf1 | GATTAGAACCACAACTCTCA | 4821 |
| Mzf1 | GCTTTCTGGGAGTCGTAGTT | 4822 |
| Mzf1 | GAGGTAATGTTTAAGTAGTC | 4823 |
| Mzf1 | GCGGGACTCCATGGTAACTA | 4824 |
| Mzf1 | GTTTGGTCCCTTAGTTACCA | 4825 |
| Mzf1 | GGAAGAAGAGAGAAGCAGAA | 4826 |
| Mzf1 | GGTAACTAAGGGACCAAACC | 4827 |
| Nab1 | GTAAGGTAACAATTATGGAG | 4828 |
| Nab1 | GTGTCCTCAGAACTTAACTT | 4829 |
| Nab1 | GTCCTTCCTTGTTTATGTTC | 4830 |
| Nab1 | GGAGTTGCTGTTGAAGTCAC | 4831 |
| Nab1 | GGAGCATAAACACTGACAAT | 4832 |
| Nab1 | GAGTTTAGGAATGGGAAGGA | 4833 |
| Nab1 | GCCCAGAACATAAACAAGGA | 4834 |
| Nab1 | GGTATCCTTAAGGCTCTTTC | 4335 |
| Nab1 | GTGGAAAGGTAGAGGTTAAT | 4836 |
| Nab1 | GGCCAGCCAGGAAGTGGGAA | 4537 |
| Nacc1 | GTTGGATCCTGTGAGCGGAA | 4838 |
| Nacc1 | GAGTCAAGAACAGAAGAGTG | 4839 |
| Nacc1 | GTTTGTCCGGGTGTGTGTGT | 4840 |
| Nacc1 | GGAACAGTTTAGGCTCTTTG | 4841 |
| Nacc1 | GCCTGAACCTCCACTCACTC | 4842 |
| Nacc1 | GATCACAGCACACCTGGAGG | 4843 |
| Nacc1 | GTCTGTGTGAATACAACTAC | 4844 |
| Nacc1 | GCAGTAAGGAAGGGACTTTA | 4845 |
| Nacc1 | GAGCCACTCAGACTGAGTGT | 4846 |
| Nacc1 | GAACCGAAGCGCTCGAAGCG | 4847 |
| Nanog | GTGGGAAGTTTCAGGTCAAG | 4848 |
| Nanog | GGGAAGTTTCAGGTCAAGTG | 4849 |
| Nanog | GCTTTCCCTCCCTCCCAGTC | 4850 |
| Nanog | GTGAATTCACAGGGCTGGTG | 4851 |
| Nanog | GCGCTCTGCGTTTCTCCAGC | 4852 |
| Nanog | GGAAGTTTCAGGTCAAGTGG | 4853 |
| Nanog | GGGATTAACTGTGAATTCAC | 4854 |
| Nanog | GCTGTAAGGTGACCCAGACT | 4855 |
| Nanog | GGAGGGAGGGAAAGCTTAGG | 4856 |
| Ncoa1 | GGGACGCTAAGGGACACTCT | 4857 |
| Ncoa1 | GTACCACTCACTGTTCTCTC | 4858 |
| Ncoa1 | GTGGTACTGTAAAGAAGGTG | 4859 |
| Ncoa1 | GAGAACAGGTAGAAAGAATG | 4860 |
| Ncoa1 | GACCAGGAAACAGACTCCAC | 4861 |
| Ncoa1 | GTCTTAAGGAAGTGTGAGAA | 4862 |
| Ncoa1 | GGAATGAACACAGGGATGGA | 4863 |
| Ncoa1 | GCTCATTTGTAAGCACCAGA | 4864 |
| Ncoa1 | GTCCCTTAGCGTCCCTGAGC | 4865 |
| Ncoa2 | GTCCTCAGCATCTCCCTGGC | 4866 |
| Ncoa2 | GAAGAAATCTAAGTGGCAAT | 4867 |
| Ncoa2 | GAGCGGTGACAGCGTTCGCT | 4868 |
| Ncoa2 | GCTGTAACAAATGTTAACAT | 4869 |
| Ncoa2 | GGTCTAGGGACCGTGACCTA | 4870 |
| Ncoa2 | GGGATTGCCTGACAAAGCAA | 4871 |
| Ncoa2 | GACAGGAGAAGAAATCTAAG | 4872 |
| Ncoa2 | GCTTAGTCTGGAGAATGAGA | 4873 |
| Ncoa2 | GTGCACTGAGTAACACAGCA | 4874 |
| Ncoa3 | GCAGGGATTTAAAGCCAAGT | 4875 |
| Ncoa3 | GAGGTTCTGCTGTCACCTCA | 4876 |
| Ncoa3 | GCCTGTGACTTGTGTTTCCT | 4877 |
| Ncoa3 | GATGGTGGCAAGGGCATGTG | 4878 |
| Ncoa3 | GGCAGACATGCCGCTGCTTT | 4879 |
| Ncoa3 | GAGTGAGGTCTCAGAACAGA | 4880 |
| Ncoa3 | CATGTAAAGAACAGACCACC | 4881 |
| Ncoa3 | GTACAAGAAGGCTGTGTGCA | 4882 |
| Ncoa6 | GCTCTTACATGAAGCTACTT | 4883 |
| Ncoa6 | GGAAACTACCTATAGATATT | 4884 |
| Ncoa6 | GGCTCTTACATGAAGCTACT | 4885 |
| Ncoa6 | GCTTTCCTTTCAGTGCAGGT | 4886 |
| Ncor1 | GTTTGTTCTTTCTCAGATGG | 4887 |
| Ncor1 | GCATGCTTGCTTACTGTGAG | 4888 |
| Ncor1 | GTGCCTGACCTGTTATCCTG | 4889 |
| Ncor1 | GATTCCGCCACCGAGGAGAC | 4890 |
| Ncor1 | GCCGTGGCTGTCCTGACTTG | 4891 |
| Ncor1 | GGAACTCAGCGGAACGAATG | 4892 |
| Ncor1 | GTCCAGTCATCACCATATTT | 4893 |
| Ncor1 | GTAGGAGGGTCGCTGGGTTA | 4894 |
| Ncor1 | GTTCCGCTGAGTTCCAAACC | 4895 |
| Ncor2 | GAAGGAGAAGCCATGGAGGC | 4896 |
| Ncor2 | GGCTTTGCCTTATAGAGACT | 4897 |
| Ncor2 | GGAAGTTCATTTCAGCCTTT | 4898 |
| Ncor2 | GGCAAGGTGTGCTGAGGTGG | 4899 |
| Ncor2 | GTTAAAGATCTAAGGCAGAG | 4900 |
| Nccr2 | GTAGGAGCCAGGGAGGACAA | 4901 |
| Nccr2 | GCTGGGTAGCGGCACTACTC | 4902 |
| Ncor2 | GAGCCCTCACATTGCCAGCC | 4903 |
| Ncor2 | GAGTCATCCTCGCCATCCCA | 4904 |
| Ncor2 | GTTCTAGCTTTAAGCCTGCC | 4905 |
| Neurod1 | GCATAGTTCTTGGATACCTT | 4906 |
| Neurod1 | GTTATCTCCGCTTGCCTGAC | 4907 |
| Neurod1 | GTCGCCAGTTAGAGACTCCG | 4908 |
| Neurod1 | GCGCATAAGAACAAGGCAGC | 4909 |
| Neurod1 | GGTAGGAGCAGGTGACCGTT | 4910 |
| Neurod1 | GGTCGGGCTACCTAACTCCA | 4911 |
| Neurod1 | GTAACTGCAAGGCCCTTAGA | 4912 |
| Neurod1 | GAACTATGCTGGGTAACAGT | 4913 |
| Neurod1 | GTCAGAACCTTGCCTTCTAA | 4914 |
| Neurod1 | GTGAAAGTATGTGTGTGTTG | 4915 |
| Neurod6 | GTGCATCTGGGTACCAGGGA | 4916 |
| Neurod6 | GATTAGAAGAGCCACTCTGG | 4917 |
| Neurod6 | GTGTCTGTGTGTAAACCTGG | 4918 |
| Neurod6 | GAGGGTTCATCCAGGATTCA | 4919 |
| Neurod6 | GAGAGGGAAAGTTTCATATG | 4920 |
| Neurod6 | GTAGAGCTAAAGTGAGTCTT | 4921 |
| Neurod6 | GTTGTAACATGGGAGATCCA | 4922 |
| Neurod6 | GTGTCACCGCTATGATTCTT | 4923 |
| Neurod6 | GTGCTGCTGCCACATGTCAA | 4924 |
| Neurog1 | GGCTGCTGGGAGTTGTGCAA | 4925 |
| Neurog1 | GTGCACTACTGAATCCAAGA | 4926 |
| Neurog1 | GTCAATCAGTAGCAGGCAAA | 4927 |
| Neurog1 | GATTGGCCGGCGGTAATTAC | 4928 |
| Neurog1 | GAATTGTCACAAGGTCAGAC | 4929 |
| Neurog1 | GAGCAAGATTTCAGGAGAAG | 4930 |
| Neurog1 | GCATAATTTATGCTCGCGGG | 4931 |
| Neurog1 | GCTGTCACAGGGACAGAAAG | 4932 |
| Neurog1 | GGCCCTGTATTTATTTCTTT | 4933 |
| Neurog1 | GGCTGGCTGTCTATTAAGTC | 4934 |
| Neurog2 | GAATAAAGGATGGGAACAGT | 4935 |
| Neurog2 | GTTTCCTCTCAAGTCCAGCA | 4936 |
| Neurog2 | GTATGACCTCTGCTCCGCTC | 4937 |
| Neurog2 | GTCACGTACGTGTGCCAGAC | 4938 |
| Neurog2 | GGACTTCAACACACGCCATC | 4939 |
| Neurog2 | GATGAAAGGAGAGTCTTGGG | 4940 |
| Neurog2 | GAGGGCTACGGAGCAGGATT | 4941 |
| Neurog2 | GCCAAACAGACCCTTAGTGG | 4942 |
| Neurog2 | GAAACGTGTCTATGACTGTT | 4943 |
| Neurog3 | GGGAGGTGGTAGGATTGGGT | 4944 |
| Neurog3 | GGATTCCGGACAAAGGGCAG | 4945 |
| Neurog3 | GCCCATTAGTCTCACGGGAT | 4946 |
| Neurog3 | GTGAAGCTGCTAGTCCTCTC | 4947 |
| Neurog3 | GCATGGGAGGAAGCTATGGC | 4948 |
| Neurog3 | GGGTAGACCTTCCTGTGAAC | 4949 |
| Neurog3 | GAGGACAGAGTGACCAGAGA | 4950 |
| Neurog3 | GCCCTTTAAGTCACTTTCCC | 4951 |
| Neurog3 | GACAATGTCTTAAGGCTCAC | 4952 |
| Nf1 | GTATCTTCCTATGTGGCTAA | 4953 |
| Nf1 | GCCATGCATAGTGGTGTGAC | 4954 |
| Nf1 | GGGAATTCTAGTCTCCAACT | 4955 |
| Nf1 | GGCAATGACAGCCTACGCAC | 4956 |
| Nf1 | GTCCTTCAAACTCTGGTTCT | 4957 |
| Nf1 | GGCCCAGTGGTGATCCAAGT | 4958 |
| Nf1 | GTCTCGGACTGTGATGGCTG | 4959 |
| Nf1 | GATGGTGTGTGTGTGTGTGG | 4960 |
| Nf1 | GAGCAAGAAGCCAGCAGTGA | 4961 |
| Nf1 | GAAAGGATCCCACTTCCGGT | 4962 |
| Nfat5 | GTGTCCTCCTAAGTACACCA | 4963 |
| Nfat5 | GTGTTATGGGCCAACGTGTT | 4964 |
| Nfat5 | GGGAATGGAGTTCCACAGCT | 4965 |
| Nfat5 | GTGAATGGTCGAATTTACTC | 4965 |
| Nfat5 | GCTAATGTCAATGACAGTTT | 4967 |
| Nfat5 | GTGTAATGCACACGCGTGCG | 4968 |
| Nfat5 | GTATCAGAIGTTCAGATGAA | 4969 |
| Nfat5 | GCTGATCCCGGGCTGGGAAA | 4970 |
| Nfat5 | GAGCTGATTTGTAGCCAGGA | 4971 |
| Nfat5 | GACCTGGATGTCAGCCAGGA | 4972 |
| Nfatc1 | GGGACGAAACGGGAAGGAAA | 4973 |
| Nfatc1 | GCCGCTTGTTTATGTAAACC | 4974 |
| Nfatc1 | GGACCCAGTACAGGGCTGAC | 4975 |
| Nfatc1 | GACTCCTGGGAAAGAGTTGA | 4976 |
| Nfatc1 | GACCAGCCGGACGCATTGAG | 4977 |
| Nfatc1 | GGCTAACTTGAGCATCACGT | 4978 |
| Nfatc1 | GCTAGATGCTGCTGGAAGAG | 4979 |
| Nfatc1 | GACGGAACGGATTGGAGGGT | 4980 |
| Nfatc1 | GGCCGTGGGAAAGCACCTTG | 4981 |
| Nfatc1 | GTCTTGAGACAGCCAGACCC | 4982 |
| Nfatc2 | GTCTCTTTGGAGGGTGGCCC | 4983 |
| Nfatc2 | GCTTCTGCTGGTTTCTCTCC | 4984 |
| Nfatc2 | GTTTGCACGCAGCTCCTGCA | 4985 |
| Nfatc2 | GAGATAAAGCCAGCTTTGAT | 4986 |
| Nfatc2 | GCGTAAACACATGCGTTGCC | 4987 |
| Nfatc2 | GTTTGTAGAAACCTATGCCT | 4988 |
| Nfatc2 | GGTGATGACTCACTAGCCCT | 4989 |
| Nfatc2 | GCACAGTAAGAGGAGATTGG | 4990 |
| Nfatc3 | GAAGTTGGTATGGAGGGATG | 4991 |
| Nfatc3 | GGAGCTCATGTCGAGGAAGT | 4992 |
| Nfatc3 | GGTGAAAGGAGTATGCATGT | 4993 |
| Nfatc3 | GTGAAAGGAGTATGCATGTT | 4994 |
| Nfatc3 | GCTACAGGAGTAGTAGAAAC | 4995 |
| Nfatc3 | GCGATAGGTCGGTGAGGAGG | 4996 |
| Nfatc3 | GATGGTGAGCAAGAGCTTTA | 4997 |
| Nfatc3 | GCTACTAAGTGAGCCTCAGG | 4998 |
| Nfatc3 | GCATTCAGATCAGCAGGAAG | 4999 |
| Nfatc3 | GGGAACCCACGTAGGCCAAT | 5000 |
| Nf8tt4 | GGAGAACAGACCCGGAAACT | 5001 |
| Nfatc4 | GGTCTTCCAGACGAGGGAAG | 5002 |
| Nfatc4 | GGCAGGGAGGAGAAGCTTGG | 5003 |
| Nfatc4 | GGCTCTGAGCTGCTCTGTAG | 5004 |
| Nfatc4 | GACAGTGAGGTGCCCTTTCT | 5005 |
| Nfatc4 | GTGGTGGCTAAGAACTGCAA | 5006 |
| Nfatc4 | GGAGATTTGCCAGGTTTATT | 5007 |
| Nfatc4 | GAAACACTGCCCAGGATCAA | 5008 |
| Nfatc4 | GAACTGCAAAGGCTCCTTGG | 5009 |
| Nfatc4 | GTAACCTGAGAAGAACCCAA | 5010 |
| Nfe2 | GATCCTCAAGGAGTGTGTTG | 5011 |
| Nfe2 | GGGAATATGGAGGCAGGATG | 5012 |
| Nfe2 | GACAGAGCTCTGCCTTGGGA | 5013 |
| Nfe2 | GACACTATGGGAACTTGCTA | 5014 |
| Nfe2 | GGGCAATTTCCGCCAGAACT | 5015 |
| Nfe2 | GAAGTGGGCTGTAATGCCTC | 5016 |
| Nfe2 | GCAAATTGGACTCAGATACC | 5017 |
| Nfe2 | GTCTATGCAATCCACTCAGG | 5018 |
| Nfe2 | GGGATGGCTTTATAGCAAGA | 5019 |
| Nfe2 | GTGTCTCCTAAAGACCGACA | 5020 |
| Nfe2l1 | GTGGGTAACTGGCATATCTG | 5021 |
| Nfe2l1 | GAATTGTTGGTCATTGTGAT | 5022 |
| Nfe2l1 | GGGTGGTGCAGTGAGAGTCC | 5023 |
| Nfe2l1 | GACTAGCCATCGTCTTCTTA | 5024 |
| Nfe2l1 | GTAAACTCCCTTTAGCTCCT | 5025 |
| Nfe2l1 | GGCAGCCTAGGTAACAAGTT | 5026 |
| Nfe2l1 | GCTAGGTAACAGGCGGTGGG | 5027 |
| Nfe2l1 | GACCCTCAAGGACGGAATCT | 5028 |
| Nfe2l1 | GGGTACCGGTTTCCGTTGCC | 5029 |
| Nfe2l1 | GCAGCTAGGTAACAGGCGGT | 5030 |
| Nfe2l2 | GATGTTTGTATGCGACAGTG | 5031 |
| Nfe2l2 | GGTTCTGCAGGTCCAAATCA | 5032 |
| Nfe2l2 | GTGAGACATCTAAGGCAAGA | 5033 |
| Nfe2l2 | GAGATTACTGTATGACCTTG | 5034 |
| Nfe2l2 | GGCATTCCTTTCTTCACCTC | 5035 |
| Nfe2l2 | GAGAGGAGGATCAACAGTGG | 5036 |
| Nfe2l2 | GGCAGTTAAAGAAGTATGTT | 5037 |
| Nfe2l2 | GCTCTCCTGCCGACAGAGGT | 5038 |
| Nfe2l2 | GGAGCTGCCACTCCCTGATT | 5039 |
| Nfe2l2 | GGGCACGTGGGAGAAGTGGA | 5040 |
| Nfe2l3 | GTGGTCCAGGTCACTACCAC | 5041 |
| Nfe2l3 | GGAAAGTTGGAGAAGTTGGG | 5042 |
| Nfe2l3 | GGGTGGGAGTGGAGGAAAGT | 5043 |
| Nfe2l3 | GTGCTGCAATGCTGGCAGCT | 5044 |
| Nfe2l3 | GCTGCCAGCATTGCAGCACT | 5045 |
| Nfe2l3 | GTCACTACCACAGGGCTGCC | 5046 |
| Nfe2l3 | GCAATCTCCAACAGCACACG | 5047 |
| Nfe2l3 | GGAGAAGTTGGGAGGAGACA | 5048 |
| Nfe2l3 | GGACACACTCATATCTGTTC | 5049 |
| Nfia | GATAGGAGAGAAAGCAGGAG | 5050 |
| Nfia | GCAAAGGCTGTAGTTGGAAC | 5051 |
| Nfia | GCCAACTGAACCAGAAAGCA | 5052 |
| Nfia | GTAGTTATATAGGCTAGTGT | 5053 |
| Nfia | GATGCCGTAGAAATGAATTC | 5054 |
| Nfia | GTTCACAATCTTGAGGAGGG | 5055 |
| Nfia | GAAACAACAGTGGTTTAGCT | 5056 |
| Nfia | GGATTTACCCTTCCTAACAA | 5057 |
| Nfia | GCATAGGACATTCGGGATCC | 5058 |
| Nfia | GTTTGCTTAAGCACATCCTG | 5059 |
| Nfib | GTTTGAGCATTTCCCTAATG | 5060 |
| Nfib | GCTCCATGTCGCCCTAGCTT | 5061 |
| Nfib | GAAATAACCTCTCCCTGGGC | 5062 |
| Nfib | GAACTTGATTCCCGGGACCC | 5063 |
| Nfib | GGGTGCCAGGATTTCGCTGG | 5064 |
| Nfib | GTTAAAGCTGGTATTATCAG | 5065 |
| Nfib | GAACGCGCGTTTGCAGGAGG | 5066 |
| Nfib | GAAGCAATAACAGTGTGGTG | 5067 |
| Nfib | GAGAAAGCAGAGGTCTCAGG | 5068 |
| Nfib | GAGAGAGTGCCCGCGCGAAA | 5069 |
| Nfic | GGGCGCGCATCCAATCTGAC | 5070 |
| Nfic | GTGCTGTCCCTAATATAGGG | 5071 |
| Nfic | GACTTGTGAGTGGACACTGG | 5072 |
| Nfic | GTCACTCACAGGCATCTCCT | 5073 |
| Nfic | GTTGGCTCGGTAGTGACACC | 5074 |
| Nfic | GCTGCTGCAGGGACTCAGGT | 5075 |
| Nfic | GAGCTATCCATTTGTAGAGG | 5076 |
| Nfic | GGTGGTTTGGTCAGTATCCG | 5077 |
| Nfic | GACATGGGATGTGAGGGCTG | 5078 |
| Nfic | GTCACTAACCCAGCAGGGTT | 5079 |
| Nfil3 | GAAATGGGAGACAGAGCATC | 5080 |
| Nfil3 | GTGCGTCACTGTCAGGAATA | 5081 |
| Nfil3 | GATCCCTAAGTAGGTAGAAT | 5082 |
| Nfil3 | GAAATGTCCCGCTCCTCTCC | 5083 |
| Nfil3 | GCGTCCGGTGTTACACCCTG | 5084 |
| Nfil3 | GAACTTGCCTGACTCACCCA | 5085 |
| Nfil3 | GAGGATAAATCTCCTTTCAC | 5086 |
| Nfil3 | GGTGGCAAGGTCCTTGAGCT | 5087 |
| Nfil3 | GTTTCCCGGAGAGLCACAGA | 5088 |
| Nfix | GGGAGGAATAGAGCAAATGA | 5089 |
| Nfix | GCCATTGAACAGAAAGGCCA | 5090 |
| Nfix | GGGAAAGTCCACACAAGTTG | 5091 |
| Nfix | GGCCATGTTTGCAATTGTTT | 5092 |
| Nfix | GGCGCTGCCTTCCCGTATAT | 5093 |
| Nfix | GTGCTGCCCGTTTAGGGTAT | 5094 |
| Nfix | GCGTCCATGCTCATAAACCA | 5095 |
| Nfix | GTCCCAAACCTCTGAGATGG | 5096 |
| Nfix | GTAGGACATAGAGAACTGTT | 5097 |
| Nfix | GAAGGCAGAGGGCCTTTAGG | 5098 |
| Nfkb1 | GACTCTCTAATATACAGTGT | 5099 |
| Nfkb1 | GAAATTGTAACCTACGGGCC | 5100 |
| Nfkb1 | GATTTGTAGAAGTTTGAGTG | 5101 |
| Nfkb1 | GCCATTACTGAGGCGTTGAA | 5102 |
| Nfkb1 | GATCGCTCCATAGAGCGGAC | 5103 |
| Nfkb1 | GTCTCACTACTGAGTTCAAG | 5104 |
| Nfkb1 | GTTGATTACAGGGCTCTTTA | 5105 |
| Nfkb1 | GTTCTAACCAATGATGCCTA | 5106 |
| Nfkb1 | GAGGCTCTGGAGAACTCCCA | 5107 |
| Nfkb1 | GTTTGGTTGTTCCATGGCAG | 5108 |
| Nfkb2 | GTTTGCTCCAGGCTGCGGAG | 5109 |
| Nfkb2 | GATGTTTATTCTGTAAGTGG | 5110 |
| Nfkb2 | GAGGGACCTCCTAGCTGGGA | 5111 |
| Nfkb2 | GAGGACTTTAGATGACAGGC | 5112 |
| Nfkb2 | GGGACCTCCTAGCTGGGAAG | 5113 |
| Nfkb2 | GCTGTGCACAGGCAAGCTAA | 5114 |
| Nfkb2 | GCCTTTCAAGTCAAATAGTT | 5115 |
| Nfkb2 | GTGCGCTGTGAGTGCGTGTG | 5116 |
| Nfkb2 | GGACTTTAGATGACAGGCTG | 5117 |
| Nfkb2 | GACAGGGTGGTGTGAAACTT | 5118 |
| Nfkbib | GTTCTTTGGGTAGAAAGGAA | 5119 |
| Nfkbib | GCCGGCGGCCATATTGATAA | 5120 |
| Nfkbib | GTACAGGCCTGAGAGCACGA | 5121 |
| Nfkbib | GGAGATGCAGTGAAGGTAGG | 5122 |
| Nfkbib | GAGCTGTOACAGCCTGCTGT | 5123 |
| Nfkbib | GGCGAGACTGGACTGAAGGA | 5124 |
| Nfkbib | GCATTCAGTGGTTGTAGGCA | 5125 |
| Nfkbib | GTCGTATAAGTGAAGTGATA | 5126 |
| Nfkbib | GGAGTACCGGGCAAACTCTG | 5127 |
| Nfkbib | GGGAGACAGGATCTACCTGA | 5128 |
| Nfya | GCGGTGTCAAATCCAGGAAG | 5129 |
| Nfya | GCGCCCGCTCTCGGTAGTAA | 5130 |
| Nfya | GCCTGCGTGGTATATAATTC | 5131 |
| Nfya | GTAGCTATTCTGAAGAGGGA | 5132 |
| Nfya | GCCCTCCTCCAAGCAGGGAA | 5133 |
| Nfya | GTTGCCCTCCTTAGGGTAGG | 5134 |
| Nfya | GGGTCATCCTTCACCTGCAA | 5135 |
| Nfya | GAGAAGCAGGGTTGAAGCAG | 5136 |
| Nfya | GCTTAGAAATAGGTGGGCAG | 5137 |
| Nfya | GAGGATTGTCGAATGGGTGC | 5138 |
| Nfyb | GCTACCATTCTCCCTTGTGG | 5139 |
| Nfyb | GGTACAGGGTGGAAGTCGGC | 5140 |
| Nfyb | GAGGAGGGTGTCCTAGAATT | 5141 |
| Nfyb | GCTGTGTGCCTGAGGTGGCT | 5142 |
| Nfyb | GGAAGGCCTTAAATGCACAG | 5143 |
| Nfyb | GGTAGTAAGCCAACTTGGTA | 5144 |
| Nfyb | GTAAATCTGGCTAGTAAGAA | 5145 |
| Nfyb | GGATGAGAACGCCGGCCTCT | 5146 |
| Nfyb | GGTAAATCTGGCTAGTAAGA | 5147 |
| Nfyc | GCTGCGCACTACGCGTTGCT | 5148 |
| Myc | GGAAACAGTCATGCTGTTAC | 5149 |
| Nfyc | GTCTACAGTAATATCAGCTA | 5150 |
| Nfyc | GGGTTGTGCATTGAGGCAAC | 5151 |
| Nfyc | GCAGGAAGGGCTATAGCCCA | 5152 |
| Nfyc | GTAATACATGCCTCTAATCT | 5153 |
| Nfyc | GATAGTCTGTGATGTAATCT | 5154 |
| Nfyc | GGAGCAGGAGGTCTTTCCCA | 5155 |
| Nfyc | GAAACATTCTAGGGTGCTGA | 5156 |
| Nfyc | GTAATTTCACTGCTTCTGAT | 5157 |
| Nhlh1 | GGTCCTAGTCCTCCTTATCC | 5158 |
| Nhlh1 | GACCCAGGTCCCGCAGACTT | 5159 |
| Nhlh1 | GCACAGTGAGCTACAGTATA | 5160 |
| Nhlh1 | GCAAAGATGAGGAGAGAGGA | 5161 |
| Nhlh1 | GAAGAACCTTGAGAGACCAC | 5162 |
| Nhlh1 | GTTCATCCCAACTCCCTACA | 5163 |
| Nhlh1 | GGAACGAAGGCCTGAGGAGG | 5164 |
| Nhlh1 | GTGTTAAGGCGTCATCCAAA | 5165 |
| Nhlh1 | GGGAAGACAGAGAGGTAGGG | 5166 |
| Nhlh2 | GGAGGTGGAAGATCCAAGAA | 5167 |
| Nhlh2 | GGAGTGACGATGTGGGAGAG | 5168 |
| Nhlh2 | GTCCCTGGTCACCCTCGTGT | 5169 |
| Nhlh2 | GAAGGCTGGGCATCTGTGAG | 5170 |
| Nhlh2 | GAGAGGAAGGTTTCCCAGCC | 5171 |
| Nhlh2 | GAAAGCACAGCTGCTAGGAT | 5172 |
| Nh1h2 | GGAACAGGGAGACAGGAGGT | 5173 |
| Nhlh2 | GTGTCACAGCAAGCTGATGA | 5174 |
| Nhlh2 | GGGAGCCAGGAGTGACGATG | 5175 |
| Nhlh2 | GGGATACCTGGGTTGAGCAG | 5176 |
| Nhlh2 | GAGGATGCTCAAACCATGGC | 5177 |
| Nkx1-1 | GCGGGATCAGTTGGCTGTGG | 5178 |
| Nkx1-1 | GGCGGGAGTCAAAGCCAGTG | 5179 |
| Nkxl-1 | GGGAGCAAAGACCAAGATGG | 5180 |
| Nkx1-1 | GTCAAAGCCAGTGAGGATGG | 5181 |
| Nkx1-1 | GCTCATGTCAGAATATTGAG | 5182 |
| Nkx1-1 | GAAGTGGAAGGAGGAGCAGA | 5183 |
| Nkx1-1 | GTCCCACCTGGGTCCTTCAG | 5184 |
| Nkx1-1 | GAGCCCGGCTTTGGAGGATG | 5185 |
| Nkx1-1 | GACCTGCCTGCTGATGGGTA | 5186 |
| Nkx1-1 | GTGGACTGTGCTCTGGCTCC | 5187 |
| Nkx1-2 | GTAAGAAGTAGGAAGAGGAG | 5188 |
| Nkx1-2 | GATTTGCACGCATTGTCCCT | 5189 |
| Nkx1-2 | GGATATGTGTGTGTGTGCGG | 5190 |
| Nkx1-2 | GGCTGTCCTCCCTGGAGACT | 5191 |
| Nkx1-2 | GGCAAGGCTTTAAAGTCGGC | 5192 |
| Nkx1-2 | GCCCTAAGCCTTCAGCTCTC | 5193 |
| Nkx1-2 | GGCATCACATCCCAAAGCAG | 5194 |
| Nkx1-2 | GCAATCAGGTCTGGCCTCTG | 5195 |
| Nkx1-2 | GGACAATCGCTTGAGAAGCC | 5196 |
| Nkx1-2 | GTTCTGTGTGATCGTGGCTG | 5197 |
| Nkx2-1 | GTGTGCATACACACTGTATG | 5198 |
| Nkx2-1 | GGATTAGCTAGGTTAGTGCT | 5199 |
| Nkx2-1 | GTGCATACACACTGTATGTG | 5200 |
| Nkx2-1 | GTGTCTAGGAGGCACCTGCC | 5201 |
| Nkx2-1 | GGTGGTCATAGGAACACCAA | 5202 |
| Nkx2-1 | GACTCAGTTCCACTCTGCAA | 5203 |
| Nkx2-1 | GTGTCAGTGACTTAAATAAT | 5204 |
| Nkx2-1 | GCGTTGTGTCTCTGTAGCTA | 5205 |
| Nkx2-1 | GGCAAGTGGTAGATCTGGTT | 5206 |
| Nkx2-1 | GCAGGAGGCACCAGCCATGA | 5207 |
| Nkx2-2 | GTTGGGAGGGTAGAGGGCCT | 5208 |
| Nkx2-2 | GGCCCAAGCAGCTGTGAGCT | 5209 |
| Nkx2-2 | GGTTGAATGCCATGACAACT | 5210 |
| Nkx2-2 | GTTCTGCTTCGCCTGGACTA | 5211 |
| Nkx2-2 | GGTTTCCTTAATATTGTGGA | 5212 |
| Nkx2-2 | GTCACAAGGCTCTAGAAACC | 5213 |
| Nkx2-2 | GGTGAAGACCCAGAAATCCA | 5214 |
| Nkx2-2 | GGGCGGTCTAGAGAAGGGAG | 5215 |
| Nkx2-2 | GCTCTAGCAGTGGCAGGGTT | 5216 |
| Nkx2-2 | GAGCACTGCTTGGTTGGACC | 5217 |
| Nkx2-3 | GGTTCACCCACCCAGGGTTC | 5218 |
| Nkx2-3 | GGAGAGGAGTGTTGTATCTG | 5219 |
| Nkx2-3 | GAGCCGAATTGCCTCTTCTA | 5220 |
| Nkx2-3 | GTTTCAGAAAGTTGAGGCCT | 5221 |
| Nkx2-3 | GTCTGAIGGAGACCACCTTC | 5222 |
| Nkx2-3 | GGGTGGGTGGAAGTCTCCAG | 5223 |
| Nkx2-3 | GCCTGGCCTGAGTCAGTATT | 5224 |
| Nkx2-3 | GCTGTCTGCTCCCTACCTGC | 5225 |
| Nkx2-3 | GGGTACCCAACAAGGATCCC | 5226 |
| Nkx2-3 | GGAAAGAAAGAACTGCGGGT | 5227 |
| Nkx2-4 | GTGAGCTTGATAATAGACTC | 5228 |
| Nkx2-4 | GTATGGTGCTCCTACTCTCA | 5229 |
| Nkx2-4 | GGACTTGGGACACTTGAGCT | 5230 |
| Nkx2-4 | GTGAGGAGAGGAAATGGGAA | 5231 |
| Nkx2-4 | GTGTTGTGAGGAGAGGAAAT | 5232 |
| Nkx2-4 | GCGAAGGATGGAGCTAGAAA | 5233 |
| Nkx2-4 | GAGTCCAGGTTAAACTTTGG | 5234 |
| Nkx2-4 | GCAAAGAATCTGCCTGTTGT | 5235 |
| Nkx2-5 | GTTATGCTGAGTCTAAACGC | 5236 |
| Nkx2-5 | GGGAGTCCTGTTAAGTGAAT | 5237 |
| Nkx2-5 | GTTGTGCCTTTCAGAGCACA | 5238 |
| Nkx2-5 | GGGTTCTGAGCTGAATGGAA | 5239 |
| Nkx2-5 | GATCGGGCTAGAAAGGGTCT | 5240 |
| Nkx2-5 | GCTGAGTCTAAACGCAGGGT | 5241 |
| Nkx2-5 | GCGGCTGATTGCAGGAAAGG | 5242 |
| Nkx2-5 | GATTGAAGATTGGTTTGTGT | 5243 |
| Nkx2-5 | GTTGAAAGGGACAGAGACAA | 5244 |
| Nkx2-5 | GATAGTCTCCCACTCCTGCA | 5245 |
| Nkx2-6 | GGGTTTGGAGGGCTAGTTAG | 5246 |
| Nkx2-6 | GAAGGCTCAGGGTTAGCACG | 5247 |
| Nkx2-6 | GCTTAAGAGCAAAGACCTGG | 5248 |
| Nkx2-6 | GAAAGCAGGGAGCCAGCCAG | 5249 |
| Nkx2-6 | GGGTTAGCACGTGGTTTCTG | 5250 |
| NkX2-6 | GTGCTATCTAGACCTGGGAG | 5251 |
| Nkx2-6 | GAGATCAGTGGACCACTTGA | 5252 |
| Nkx2-6 | GTACAACAGAGAGCTCCCGA | 5253 |
| Nkx2-6 | GTGGTTTCTCTGGGAACCAA | 5254 |
| Nkx2-6 | GGGAGAGTGCTGTTCAAACT | 5255 |
| Nkx2-9 | GGGCTCAGTTGGGAGGACCA | 5256 |
| Nkx2-9 | GCAATGTACAAGTCTTCCTT | 5257 |
| Nkx2-9 | GGACCCTGAGTCTGGGACTC | 5258 |
| Nkx2-9 | GTCTTGGGAGAAAGCAGGAG | 5259 |
| Nkx2-9 | GTTTGAGCAGGGAAATGACC | 5260 |
| Nkx2-9 | GGCACCGACTTGGGAGATGA | 5261 |
| Nkx2-9 | GCTGAATTCGAACCTGACAA | 5262 |
| Nkx2-9 | GAGCCAGAGGAAGACTAGAA | 5263 |
| Nkx2-9 | GCACCGACTTGGGAGATGAA | 5264 |
| Nkx2-9 | GCCAGAGGCAGAGGATGCAC | 5265 |
| Nkx3-1 | GGGAAACCAGGAAAGGTTAA | 5266 |
| Nkx3-1 | GGCATAGCCACTGCACCACT | 5267 |
| Nkx3-1 | GGAATCAGAACTGAGCAGGC | 5268 |
| Nkx3-1 | GGTTAAGGGCTCATCAGGGA | 5269 |
| Nkx3-1 | GCAGTTACTCACTGTTTGGA | 5270 |
| Nkx3-1 | GGGCTCCAGGTGACCCTCAA | 5271 |
| Nkx3-1 | GTTGTCTAGATGTGTCCAGC | 5272 |
| Nkx3-1 | GGTACAGTGCTATTTCAGTT | 5273 |
| Nkx3-2 | GCTGGAGAAGGAACAGATTG | 5274 |
| Nkx3-2 | GATGTTAATTTCAGAAGCTG | 5275 |
| Nkx3-2 | GCGAGGAATTGGAAAGCATT | 5276 |
| Nkx3-2 | GTGAGGAATGACACTCTGAT | 5277 |
| Nkx3-2 | GTGAGCTCTGGACATGCTGA | 5278 |
| Nkx3-2 | GTGTGATGGCCTGTGGACAT | 5279 |
| Nkx3-2 | GGACCCTGCAGCATCTTCAT | 5280 |
| Nkx3-2 | GCGAGGCGGACGACTTTGAC | 5281 |
| Nkx3-2 | GGAATGACACTCTGATGGGA | 5282 |
| Nkx3-2 | GTGGATTGGCTGGTTCCAAC | 5283 |
| Nkx6-1 | GCCTGCCAGTCTCTAGGCTC | 5284 |
| Nkx6-1 | GTGATAATGATCTAGGGAGT | 5285 |
| Nkx6-1 | GGTTTGAAAGCAGCAAACCC | 5286 |
| Nkx6-1 | GAGCTAATGGAGCAGGCAGG | 5287 |
| Nkx6-1 | GCCTCTAGCCAGGTGCTGTC | 5288 |
| Nkx6-1 | GTGTCACTGACTGCCCTTTC | 5289 |
| Nkx6-1 | GGGTTTAGGTAGCAGAGGGC | 5290 |
| Nkx6-1 | GGTCCAGACACCGTTGGAGG | 5291 |
| Nkx6-1 | GATCATTATCACTTATGAGG | 5292 |
| Nkx6-1 | GGGCAGTTGATACACCAGTG | 5293 |
| Nkx6-2 | GGATGAATGAAGCGGGAGTG | 5294 |
| Nkx6-2 | GAGACAGGGTAGGTGTGCTC | 5295 |
| Nkx6-2 | GCTTAGTTCAGGGAAGAGCC | 5296 |
| Nkx6-2 | GTGGGCTGTTGTGAACTTGT | 5297 |
| Nkx6-2 | GTGCCTAGTGGTCCTGTCCT | 5298 |
| Nkx6-2 | GGCGAACTATGAGACAGGGT | 5299 |
| Nkx6-2 | GTTCAGGGAAGAGCCTGGGA | 5300 |
| Nkx6-2 | GGGCGAATGGAAATTTGTTA | 5301 |
| Nkx6-2 | GCATCTCCGTAGGTGGGCTG | 5302 |
| Nkx6-2 | GGTCCTGGCGATTTAAGCAG | 5303 |
| Nkx6-3 | GAGCAATCACTATTCTCTGG | 5304 |
| Nkx6-3 | GAACAGAGCTACACAGAAAG | 5305 |
| Nkx6-3 | GGCATTCCAcTGAAGAATGG | 5306 |
| Nkx6-3 | GAGACCTAAGCAGGGCAGTC | 5307 |
| Nkx6-3 | GATGAGCCAAGAAGAAGCGA | 5308 |
| Nkx6-3 | GTCCACCAATGCCCAGATCC | 5309 |
| Nkx6-3 | GGCTCATCTTTGGGAGTTCG | 5310 |
| Nkx6-3 | GCGTCACATTCATTCCGACA | 5311 |
| Nkx6-3 | GTAGGGACTGGAGGCTCCTG | 5312 |
| Nobox | GAGAGACTTCTGACAGGAGT | 5313 |
| Nobox | GGGTCAGCACTTCTAAGAAG | 5314 |
| Nobox | GACTTCCAATAAGCTGCTGT | 5315 |
| Nobox | GTTTAGTCTCCTCCAGGCCT | 5316 |
| Nobox | GCTTCCCAAGGAAGGCCTTG | 5317 |
| Nobox | GCCTGCTTGATGGAAAGGTA | 5318 |
| Nobox | GGAGCAGAACAGCAATGGAA | 5319 |
| Nobox | GCTCATATTCAAGGGTCAAG | 5320 |
| Nobox | GCATGGTGCTCTTGCTGGTG | 5321 |
| Nov | GCTGGAGAGTCAAGTCAAGC | 5322 |
| Nov | GGTTGGAACTGTGAGGGCGG | 5323 |
| Nov | GGAGCCATATGAGCTGGGCA | 5324 |
| Nov | GGAGGCGTCCATCAGGTTAG | 5325 |
| Nov | GCTGATTCTTGACCCTCTCC | 5326 |
| Nov | GCAAAGTTTAGGCAGAGGTA | 5327 |
| Nov | GTGCCATCTTGGAGTATTAG | 5328 |
| Nov | GACTAAGCTTTGCCTAAAGG | 5329 |
| Nov | GGGAAGAAAGGTGTAATTTA | 5330 |
| Npas1 | GCTGGCAGAGCTTCCTGATG | 5331 |
| Npas1 | GAGGCATAGAGACAAGACCT | 5332 |
| Npas1 | GTGAGGATGCICCTACACTC | 5333 |
| Npas1 | GGCATCCTGGAATTCTCACT | 5334 |
| Npas1 | GTTACAGAACCTTCCAACAT | 5335 |
| Npas1 | GCGATCGTGGTGGGACTCCA | 5336 |
| Npas1 | GTGTGCTCACACGCATTCCA | 5337 |
| Npas1 | GGGAACTATCCAGCAGGCAG | 5338 |
| Npas1 | GTGACAGTTAAAGCTGCGCA | 5339 |
| Npas2 | GTGTTTCTTCTCACCCAGGA | 5340 |
| Npas2 | GAGGTGAGTCCTGCGCACTC | 5341 |
| Npas2 | GATCTCTGGACGCCAGTAGA | 5342 |
| Npas2 | GGCAGGGTTTGTAGGATGCT | 5343 |
| Npas2 | GTCCTGGCTCATGGTGTTCT | 5344 |
| Npas2 | GGCTAGACCAGCCGGAAGAG | 5345 |
| Npas2 | GGTGCAGGTCCAGTTTGCAC | 5346 |
| Npas2 | GATAGGTAGCCAGGAGCCAA | 5347 |
| Npas2 | GTCAGAAACAAGCCTGAGGA | 5348 |
| Npas2 | GTGAGAGTGAACGCTGTTCG | 5349 |
| Npas3 | GAGCATTTCTACCTGGGTTA | 5350 |
| Npas3 | ACAGTCACAGGGAGACTGGG | 5351 |
| Npas3 | GAAAGATTGCATGGCACTAC | 5352 |
| Npas3 | GGAGAACTTGATAGTTATCT | 5353 |
| Npas3 | GTGAGCGGAAGAGTTGGTCT | 5354 |
| Npas3 | GGGTTTCACTGAGCTAGGGT | 5355 |
| Npas3 | GCCTGCACAGAGCAAAGGGC | 5356 |
| Npas3 | GACGCCTGCCTTTCATTAGG | 5357 |
| Npas3 | GTTTCCAGAATCATCAGGGT | 5358 |
| Npas3 | GAAATAACCACCATCCGGGC | 5359 |
| Nr0b1 | GATGCTGGATCGAGGAGCTG | 5360 |
| Nr0b1 | GTTACTATCCTATGATGGTT | 5361 |
| Nr0b1 | GAGGTCAGAGTCTAAGTTAA | 5362 |
| Nr0b1 | GACCTTAAGGTGCAGGACTT | 5363 |
| Nr0b1 | GGGACACTATCAGGAATAAA | 5364 |
| Nr0b1 | GAACACTGAGCCAATGGGTA | 5365 |
| Nr0b1 | GGTGACGAAGGCCAGCAATT | 5366 |
| Nr0b1 | GGAACACTGAGCCAATGGGT | 5367 |
| Nr0b1 | GTGGAGTGAAGAAGGAAAGG | 5368 |
| Nr0b1 | GGATGCTGGATCGAGGAGCT | 5369 |
| Nr0b2 | GAGACAAATGTCCAGGACAG | 5370 |
| Nr0b2 | GAGAGAACAAACAGAGCTCA | 5371 |
| Nr0b2 | GCGATAAGCCACTTCCAGGC | 5372 |
| Nr0b2 | GTTCTACCCATACTGTAGGT | 5373 |
| Nr0b2 | GAACCCTGGTCTTATGTGCA | 5374 |
| Nr0b2 | GTCTCTAGIGGTCAGAGGTA | 5375 |
| Nr0b2 | GGTTCTACCCATACTGTAGG | 5376 |
| Nr0b2 | GTGACTGCTCCTTTCCATCA | 5377 |
| Nr0b2 | GTATGGCCCACCTACAGTAT | 5378 |
| Nr0b2 | GAGACTGTAAGGTCTTCCTG | 5379 |
| Nr1d1 | GGGTAGGACTGGCATAGCAC | 5380 |
| Nr1d1 | GCAAGGGCATGTGAATTCCT | 5381 |
| Nr1d1 | GGGAGGAGCTAAGACAAACA | 5382 |
| Nr1d1 | GAGGTAGTACTGGGACTAGG | 5383 |
| Nr1d1 | GTGAGAAACACAGAGGCCTG | 5384 |
| Nr1d1 | GTTGGCTGGGAGGAGGGAGA | 5385 |
| Nr1d1 | GCTCCTCCCAGTTCCTCCCA | 5386 |
| Nr1d1 | GATCTCAACGTGCCGGCTGC | 5367 |
| Nr1d1 | GCTGGGAGGAAGGGAAGGAG | 5388 |
| Nr1d1 | GGCAAGGGCATGTGAATTCC | 5389 |
| Nr1d2 | GCTCAGAGTCCTGGAAAGCT | 5390 |
| Nr1d2 | GCAGTAACCATGTGGGACCA | 5391 |
| Nr1d2 | GGAGCCTGTATAGAGGAAGT | 5392 |
| Nr1d2 | GAGGTCAGGACCGCTCGTTG | 5393 |
| Nr1d2 | GGGATAAGCGGCTGCGAGAC | 5394 |
| Nr1d2 | GGTCCACGGATTGGAAGAAG | 5395 |
| Nr1d2 | GCGAGACAGGCTGGGAAGGA | 5396 |
| Nr1d2 | GAGCAAACAACCTCTAGCAG | 5397 |
| Nr1d2 | GTCACCTCCATGGTCCCACA | 5398 |
| Nr1h2 | GATTCCCAACTGTCCATAAG | 5399 |
| Nr1h2 | GCTGCGAGGAAAGTGAGGGA | 5400 |
| Nr1h2 | GACAGACTTCCGGTCTGCCA | 5401 |
| Nr1h2 | GGAGATGGCAAGATGGTTAC | 5402 |
| Nr1h2 | GAGGTTATCTGAGGTTGGAC | 5403 |
| Nr1h2 | GAGATGCTGGCCCTGGAAGC | 5404 |
| Nr1h2 | GCGTCACTTCCGGAAGTAGG | 5405 |
| Nr1h2 | GAGAGCTGCGAGGAAAGTGA | 5406 |
| Nr1h2 | GGAAGTAACTTCAGAAGCCT | 5407 |
| Nr1h3 | GAGGGAGCGCCAAGAGTAAA | 5408 |
| Nr1h3 | GGGTGAAGACAGGCAGGTGC | 5409 |
| Nr1h3 | GGGAAGGGTGAACATGGTTG | 5410 |
| Nr1h3 | GAGCCTGTGAGCAGGAAACT | 5411 |
| Nr1h3 | GACTGGGAACACGTGCAAGA | 5412 |
| Nr1h3 | GAGGCTGCTGGGATTAGGGT | 5413 |
| Nr1h3 | GAAGAGATTAGGGAGTCAGG | 5414 |
| Nr1h3 | GAGGCAGAAGCTGAAGATGG | 5415 |
| Nr1h3 | GACAGTGCTGCCTCTTCTAC | 5416 |
| Nr1h3 | GAGGTGTCTTTGGGAGGAGG | 5417 |
| Nr1h4 | GGAGGAGAAAGAAATGTATT | 5418 |
| Nr1h4 | GGGATTCTCCAAACTGCTTC | 5419 |
| Nr1h4 | GATACATGTAGAGGAGCTGA | 5420 |
| Nr1h4 | GGATGTCAGCAAATTATGGC | 5421 |
| Nr1h4 | GGAATAATTCCAACCATCAC | 5422 |
| Nr1h4 | GGATCTTTACCTTTGTAACT | 5423 |
| Nr1h4 | GCTGGAGGTTAAATGCCACA | 5424 |
| Nr1h4 | GATCAAGGTGTTTACAAAGG | 5425 |
| Nr1h4 | GTTCTTAGGAGATTAGAGGG | 5426 |
| Nr1h5 | GTCCCAGCACCTATGTTAAT | 5427 |
| Nr1h5 | GATCATTGCTGGCAAGGCAA | 5428 |
| Nr1h5 | GAACTCCTCACTTACCCTTA | 5429 |
| Nr1h5 | GATCTTGCTGCTGCGTGTCT | 5430 |
| Nr1h5 | GAGAGCTGTACAGAAAGAAC | 5431 |
| Nr1h5 | GAGCTGTACAGAAAGAACAG | 5432 |
| Nr1h5 | GCACTGCTCTGCAGAGTGTC | 5433 |
| Nr1h5 | GATGAAATCAAACGGACAGG | 5434 |
| Nr1h5 | GTCACATCTTCTCTGACGGA | 5435 |
| Nr1i2 | GGAGGAAATAGCTTCGAGAC | 5436 |
| Nr1i2 | GAAGAGCATTTCTCTCCTTT | 5437 |
| Nr1i2 | GAGTCACTCCCACGCATGGC | 5438 |
| Nr1i2 | GGACAAGACGGGCTCCATTG | 5439 |
| Nr1i2 | GGGACACTTATTTCCACGAG | 5440 |
| Nr1i2 | GAGTGACTCAGGTCCTCTCT | 5441 |
| Nr1i2 | GCCAGAGAACCAGAGAGAAT | 5442 |
| Nr1i2 | GGGAAATTGAACAAACCAGA | 5443 |
| Nr1i2 | GCTAGCTCGGGTGCTGGACT | 5444 |
| Nr1i2 | GGAGTGACTCAGGTCCTCTC | 5445 |
| Nr1i3 | GTGTTGGTTGGTGGCAGATG | 5446 |
| Nr1i3 | GTGCCTGCTGAGGTCAGAAG | 5447 |
| Nr1i3 | GTAGCATTGGGCAAGCTATG | 5448 |
| Nr1i3 | GTATCAGGGTTGGAGCCTGG | 5449 |
| Nr1i3 | GACCTCAGCAGGCACAAATA | 5450 |
| Nr1i3 | GCATGGATCCTGAATAAGCC | 5451 |
| Nr1i3 | GGATCCCACTTTCTTACGTG | 5452 |
| Nr1i3 | GCCACCAACCAACACTTCTC | 5453 |
| Nr1i3 | GGGATCCCACTTTCTTACGT | 5454 |
| Nr2c1 | GCAGGAACTGTTAACTATCT | 5455 |
| Nr2c1 | GGAGTCTGTGTAGGATAACA | 5456 |
| Nr2r1 | GACACCAGAGTTGCAGGTAT | 5457 |
| Nr2c1 | GTACCCTTCTCCCTCGAATC | 5458 |
| Nr2c1 | GCCAGTGAGGTTCATCTAAA | 5459 |
| Nr2c1 | GCAGAATCCTGAGCCGGAGG | 5460 |
| Nr2c1 | GTGCCCAAGACGGCAGAGAA | 5461 |
| Nr2c1 | GACTTAAGTCCATGAACTGG | 5462 |
| Nr2c1 | GAAACTCTGACTCAGCCTCA | 5463 |
| Nr2c1 | GCTGGAGAGAGCAGAGGCGA | 5464 |
| Nr2c2 | GGATATCACCTTACTTTGGA | 5465 |
| Nr2c2 | GGACACCTCAGGAGAGTTTA | 5466 |
| Nr2c2 | GTTCACCGGTGAAGTTAGCC | 5467 |
| Nr2c2 | GGCCGTGGCCCTCCTATAAG | 5468 |
| Nr2c2 | GGCGGGCTTGCTCTTACCTC | 5469 |
| Nr2c2 | GCTGCTCTTACCCTCAGGGT | 5470 |
| Nr2c2 | GCATGTTACTGAGCTCTCCC | 5471 |
| Nr2c2 | GAGCTCCCGGTACCTTCCTT | 5472 |
| Nr2c2 | GAAAGCTACCATCCATCCCA | 5473 |
| Nr2r2 | GGTACCTTCCTTGGGATGGA | 5474 |
| Nr2e1 | GTGGGAAAGAAAGAAGTCCT | 5475 |
| Nr2e1 | GACGAGTTGAGAGTGAATAC | 5476 |
| Nr2el | GTACACGCAATGGAGGCGAG | 5477 |
| Nr2e1 | GGGAGGAGATGGGAAGAGGG | 5478 |
| Nr2e1 | GTTCTCTCGGTGTGGAGTGG | 5479 |
| Nr2e1 | GAGTCAGCAGGCACTGCAGG | 5480 |
| Nr2e1 | GACCCGGTCCTTGGATCTGC | 5481 |
| Nr2e1 | GGTCTGACGTCAGCCATGTG | 5482 |
| Nr2e1 | GTTTGAGCTGTGCCGCGAGC | 5483 |
| Nr2e1 | GCAAAGATGTGGGCAAGTGG | 5484 |
| Nr2e3 | GAGGACACTGAGGGTCTTGA | 5485 |
| Nr2e3 | GCAGAGGGTATTGGGCAGGC | 5486 |
| Nr2e3 | GAGGGTCTTGAAGGATGGTC | 5487 |
| Nr2e3 | GCCTGCCCAATACCCTCTGC | 5488 |
| Nr2e3 | GCAGCCAAGTCAGGGCTTCA | 5489 |
| Nr2e3 | GCCGAGATGAGGCAGGACCT | 5490 |
| Nr2e3 | GAGGGTTAAGCCCACTTAGG | 5491 |
| Nr2e3 | GAGGAAGAGAGACTAGGGTA | 5492 |
| Nr2e3 | GTCGAGAGCCACCAGGTTAG | 5493 |
| Nr2e3 | GCAAACAAGTAGAGTGGGTA | 5494 |
| Nr2f1 | GGAGCCAAGAGAAGGGCTGC | 5495 |
| Nr2f1 | GTTTGGAGTTTGAGCATCCT | 5496 |
| Nr2f1 | GGAGGAGAAGAGAAAGTGAG | 5497 |
| Nr2f1 | GTAACTCCTCATATTGTTGT | 5498 |
| Nr2f1 | GTACGCAGATGATGGAGAGG | 5499 |
| Nr2f1 | GATGATGGAGAGGCGGGACA | 5500 |
| Nr2f1 | GTGTCAAGGAGCCAAGAGAA | 5501 |
| Nr2f1 | GCGCTGCCTTCCTGAATGGC | 5502 |
| Nr2f1 | GAAATGGCACAGGCGGCAGC | 5503 |
| Nr2f2 | GTCTCATCAGTTACAAAGAG | 5504 |
| Nr2f2 | GATGAGTTGCCAGGTCTAAT | 5505 |
| Nr2f2 | GACAGAGTGTGAGACAAGGA | 5506 |
| Nr2f2 | GATGCAGAGTAGGACACTGC | 5507 |
| Nr2f2 | GCCATCGAAATCAGGAGGAC | 5508 |
| Nr2f2 | GTATTATTGCCATTTGGAGC | 5509 |
| Nr2f2 | GCTCTGGTCTTTGTCTTAGA | 5510 |
| Nr2f2 | GGCTTCAAGACAGAAGTAGG | 5511 |
| Nr2f2 | GAATTCTCACAATCAACTAG | 5512 |
| Nr2f2 | GTGGGTTCTACATAATGCGC | 5513 |
| Nr2f6 | GGTTGGGTCCCAAAGGTTAG | 5514 |
| Nr2f6 | GACATTTGACCTTGTGGTTG | 5515 |
| Nr2f6 | GCTTGCTCCAGTAGAATTGG | 5516 |
| Nr2f6 | GCTTGCCTGAATTCGATTCT | 5517 |
| Nr2f6 | GTATCCAGGTGGATTCTTCT | 5518 |
| Nr2f6 | GATTCTGGGCACTGCATGAT | 5519 |
| Nr2f6 | GAAGAAAGGCTCTGGAAGAG | 5520 |
| Nr2f6 | GGCCAGAGAGAGGGCTTAGG | 5521 |
| Nr3c1 | GTCACTGCTCTTTACCAAGA | 5522 |
| Nr3c1 | GACTCTTCTGCTCAGTTTGC | 5523 |
| Nr3c1 | GGTGTTATGGTGTTGCTTTG | 5524 |
| Nr3c1 | GTCCCTGGAACTCAGAAAGA | 5525 |
| Nr3c1 | GTGATTAAGGAAGCCTTGCG | 5526 |
| Nr3c1 | GCTCTTCATAACTCCTCTCC | 5527 |
| Nr3c1 | GATCCCATAATTTACATGAA | 5528 |
| Nr3c1 | GAGGAAGGTGGAGAGAGGGC | 5529 |
| Nr3c1 | GTGTTGTTATGGTTTCAGGC | 5530 |
| Nr4a1 | GCCACCTAGGAGAAGAAGTG | 5531 |
| Nr4a1 | GCTAACGTGTAGTCTCGTTG | 5532 |
| Nr4a1 | GACCTTACCCTAGGGTACAC | 5533 |
| Nr4a1 | GGGTTCATGCTCCACATTGG | 5534 |
| Nr4a1 | GGCCTGCAAGGATGAAGTGT | 5535 |
| Nr4a1 | GAGAGGGAGCTGTTGGCACC | 5536 |
| Nr4a1 | GTGGCITCCATATTTAAACA | 5537 |
| Nr4a1 | GAGTCCTGGGCTAGTGTTGT | 5538 |
| Nr4a1 | GCAGCAGAAATCGGGAACCA | 5539 |
| Nr4a1 | GTGCAGGTCCTGTCTTCACC | 5540 |
| Nr4a2 | GACTGTCTGAAGATAGCTGC | 5541 |
| Nr4a2 | GTTCCAGGAGAGCGGGTATC | 5542 |
| Nr4a2 | GGCCACAAAGATGTAAAGAA | 5543 |
| Nr4a2 | GAGCCAAATGCCTCAGGCAT | 5544 |
| Nr4a2 | GTCAAGGCTGCCATCTAAAG | 5545 |
| Nr4a2 | GTGTGAGGACGCAAGGTCTG | 5546 |
| Nr4a2 | GACCTCTCATCCTTCGAAGC | 5547 |
| Nr4a2 | GTCCTTTCTTTACATCTTTG | 5548 |
| Nr4a2 | GTTCCTATGCCTGAGGCATT | 5549 |
| Nr4a2 | GTGAAAGGGACTGAAGGGCT | 5550 |
| Nr4a3 | GCAGGCGATGTTTCTAAATT | 5551 |
| Nr4a3 | GATTGAAGGAGGATCTTCTC | 5552 |
| Nr4a3 | GTTCGACCCTGTCTGATGCC | 5553 |
| Nr4a3 | GGCGATGTTTCTAAATTGGG | 5554 |
| Nr4a3 | GATTAGCAGCCTGCACAAAC | 5555 |
| Nr4a3 | GAGGCTGAGAGTGTAGGAGG | 5556 |
| Nr4a3 | GATAATGCCACTTATGTGTG | 5557 |
| Nr4a3 | GGAGACATGACATCTTTCCA | 5558 |
| Nr4a3 | GCGCAAGATACCCTCCAGGT | 5559 |
| Nr4a3 | GGTGCTTCACTTCTTCTTGG | 5560 |
| Nr5a1 | GATATGGTCCATTGGTAGCT | 5561 |
| Nr5a1 | GCTTGTCCCAGATCTGAGTG | 5562 |
| Nr5a1 | GTTGGTGTTTCTCTTCATTT | 5563 |
| Nr5a1 | GGCAGCGGCTTGTTAGCGAC | 5564 |
| Nr5a1 | GAGGCTGGCCATTAGAGGCC | 5565 |
| Nr5a1 | GGGCATGAGTCCACAAAGTA | 5566 |
| Nr5a1 | GCCCTTCATCCATCTACCCA | 5567 |
| Nr5a1 | GGTGTGGCTICAGGGACTTC | 5568 |
| Nr5a1 | GTTCCTCAACACTCTGGCTT | 5569 |
| Nr5a1 | GGCACCTAAACCTCAGGGAT | 5570 |
| Nr5a2 | GGTATCGGTGGTCCTAGCCT | 5571 |
| Nr5a2 | GCTAAGACTTCTTCTGTGTG | 5572 |
| Nr5a2 | GAAGAAAGCTCACTGATAGG | 5573 |
| Nr5a2 | GTATCGGTGGTCCTAGCCTA | 5574 |
| Nr5a2 | GTAGTGACAGCCCTGAGCAT | 5575 |
| Nr5a2 | GTGAGCTGTAAAGAGAACCT | 5576 |
| Nr5a2 | GCTCCTTAGTTTGAGGAAGA | 5577 |
| Nr5a2 | GAGTCACTGAGTTCAGAAGA | 5578 |
| Nr5a2 | GGGATGATCTTAAGCTGGGA | 5579 |
| Nr5a2 | GTCCTCTTATCAACCGGCAC | 5580 |
| Nr6a1 | GATGACGGTCGGCCGTAGTT | 5581 |
| Nr6a1 | GAATCAGGAAGGCTGTAGCA | 5582 |
| Nr6a1 | GAAATGTAGTCCTCCCAACG | 5583 |
| Nr6a1 | GGAAGACAGAAGAAATGGAA | 5584 |
| Nr6a1 | GATAGCGTGTATGTGAGAGT | 5585 |
| Nr6a1 | GAAGAGGCATGGGAGCAACG | 5586 |
| Nr6a1 | GCTTCGATACGGCCCATTAG | 5587 |
| Nr6a1 | GGTCCCTCTGTATTCCCAGA | 5588 |
| Nr6al | GCTCTCACATACAAATGAAG | 5589 |
| Nr6a1 | GGCCTGTTTGCCTCTCTACA | 5590 |
| Nrf1 | GGAGCTAATGCAGAIGTCGC | 5591 |
| Nrf1 | GTTTGGAGAGATGAAATGAA | 5592 |
| Nrf1 | GTTGTGTTATTTCCCTGTTT | 5593 |
| Nrf1 | GTTTGCTAACAGGTGCAcTT | 5594 |
| Nrf1 | GTTAATGCTGTCTGACACTA | 5595 |
| Nrf1 | GCAATGCCCAGAACCCAGGC | 5596 |
| Nrf1 | GTCTTACACAATCTAGGCGC | 5597 |
| Nrf1 | GATTCAGTGTGCACTCTCCA | 5598 |
| Nrf1 | GGTTACTACTATCCAGTCTT | 5599 |
| Nrl | GCACCTATTTAAACAGCTTC | 5600 |
| Nrl | GTATGATTCTCAGGGACCAG | 5601 |
| Nrl | GTCGGAGTATCTTTGTGCCT | 5602 |
| Nrl | GGCAGGTTTAAACATCTCCT | 5603 |
| Nrl | GGGATAGTAGGACTTAGCCA | 5604 |
| Nrl | GACGAGTCAGGGTGAAGGTA | 5605 |
| Nrl | GCCTCATCCAATAAGATGAA | 5606 |
| Nrl | GCGTATATGTCTCCTTGACA | 5607 |
| Nrl | GTCAGGGTGAAGGTAGGGCA | 5608 |
| Nrl | GGCTGAGAATTGTGTTTCCA | 5609 |
| Ntrk1 | GCCTGCCTCTTATCAGTCAG | 5610 |
| Ntrk1 | GGTTTAAACTCAGACTTTAC | 5611 |
| Ntrk1 | GGGACATTAGGAAGGCGAGC | 5612 |
| Ntrk1 | GGTGTTCTGGAGGGAGATGG | 5613 |
| Ntrk1 | GCCACTTCACACTGGAACCT | 5614 |
| Ntrk1 | GGCGGAGAGAACAGAGAAGT | 5615 |
| Ntrk1 | GCTGTCCTTGGGATCTGGCC | 5616 |
| Ntrk1 | GTATCCTCCAGGGAGAAAGG | 5617 |
| Ntrk1 | GGATACCTGGAAGACAACCA | 5618 |
| Ntrk1 | GCGCAAGACTTGCCATTTAG | 5619 |
| Numb | GGGACTCGACCACTAAGTTT | 5620 |
| Numb | GGGCGGTAAAGAGAGGATGA | 5621 |
| Numb | GGGCGGTAAAGAGAGGATGA | 5621 |
| Numb | GCTATTGTTTCCGATCTGCT | 5623 |
| Numb | GGTAATCCTCCTTTGTCAGT | 5624 |
| Numb | GAGTCAACCAATCGCAGCAG | 5625 |
| Numb | GGTTCCAGAAGATAACTAGG | 5626 |
| Numb | GCACACTTAGAACTAACCAA | 5627 |
| Numb | GATTTCTAAGAGGCCCTGTG | 5628 |
| Numb | GCTTGGAAGTGGTAGTGGTG | 5629 |
| Obox3 | GCTTCAGGAAGGGCCATATT | 5630 |
| Obox5 | GGGTGGAGCCAACGTGAAGG | 5631 |
| Obox6 | GCAGACTGAGCGGAGCTTCT | 5632 |
| Obox6 | GGCTGAATGGTGTTACAGCC | 5633 |
| Obox6 | GGAGAGCTTGCTGATGAATA | 5634 |
| Obox6 | GCTGATGAATAGGGTGAATT | 5635 |
| Obox6 | GGGAGAGCTTGCTGATGAAT | 5636 |
| Obox6 | GTGGAAGAGCCCTGCATTGG | 5637 |
| Obox6 | GTGGCACCAAAGGGTGGGTG | 5638 |
| Obox6 | GAGGCTTGTATGTATAAGGC | 5639 |
| Obox6 | GATGGTTTCCTAAGTGTGAA | 5640 |
| Olig1 | GGAGAGAGCTGAAGGGATAA | 5641 |
| Olig1 | GGTCAGGTAGACACACACAT | 5642 |
| Olig1 | GGAGCCCACTTGGGAACAGA | 5643 |
| Olig1 | GTCCTCCTCCCACGGCAGAA | 5644 |
| Olig1 | GAGCACAATGGGATTCCTTG | 5645 |
| Olig1 | GGAAGCTTGGCCAGGAAGAG | 5646 |
| Olig1 | GGGCAAGGGAGGGAGCTTTA | 5647 |
| Olig1 | GTGAGCTCAGATAAAGGCGG | 5648 |
| Olig1 | GTTGCCAGAGAGGGTTATCG | 5649 |
| Olig1 | GTAGAGACAAACAGGTGCTC | 5650 |
| Olig2 | GTCCACCCTCGAGTGTCAGT | 5651 |
| Olig2 | GTTCACTTGCCTAGGCTAAT | 5652 |
| Olig2 | GGATCTGGGTGAATTGCCTG | 5653 |
| Olig2 | GCTTGCTGAAATCAATTCCT | 5654 |
| Olig2 | GATGTTGGAAGTTCAGTGGC | 5655 |
| Olig2 | GACAGATTCTGCTAATTGAA | 5656 |
| Olig2 | GTCACTGTAGCGTCAGGCCA | 5657 |
| Olig2 | GTGACCCTGCCTACCGTGGA | 5698 |
| Olig2 | GGCATGGCCTTGGAGAGCAC | 5699 |
| Olig2 | GTGGTCCTCACTCTCTAAGT | 5660 |
| Onecut1 | GCTTTCTCAGCGGCGCCGAA | 5661 |
| Onecut1 | GAGGATCATGGATGGCAGTT | 5662 |
| Onecut1 | GGAACTAGCAACTCAGACTC | 5663 |
| Onecut1 | GAACTAGCAACTCAGACTCA | 5664 |
| Onecut1 | GCCCGGGTTCAATTCCGGAT | 5665 |
| Onecut1 | GACCAAGCTGGCTTGAAGTA | 5666 |
| Onecut1 | GGTGTGGTCAACCCAGGGTG | 5667 |
| Onecut1 | GAGTCTGAGTTGCTAGTTCC | 5668 |
| Onecut1 | GCACATCTGTCCTTTCTCCA | 5669 |
| Onecut1 | GATTTCAGGTTACCACACCC | 5670 |
| Onecut2 | GGCGCCGACGTCTTCTGTTT | 5671 |
| Onecut2 | GCCCTACAAACTTCTCCTGG | 5672 |
| Onecut2 | GGAGATTTCCGCGAACTGTG | 5673 |
| Onecut2 | GTCTTCTTGGGACTAGAGAA | 5674 |
| Onecut2 | GTGGCTGAAGACAGCCAGAG | 5675 |
| Onecut2 | GCTCCTAGAACCAAGCATCA | 5676 |
| Onecut2 | GAAGACACATACAGTATTGT | 5677 |
| Onecut2 | GCTGCCAATGGCTATAGAAG | 5678 |
| Onecut2 | GTGAGCGGGAGCTGTCCAGA | 5679 |
| Onecut2 | GGGAAGAAGGGAGGAGAAGG | 5680 |
| Osr1 | GAGTTCTGTTCTGGAGCTTT | 5681 |
| Osr1 | GTTTCCCAATGCGCAGGCGC | 5682 |
| Osr1 | GTTACTAAGGGATTGCTTCT | 5683 |
| Osr1 | GCACAGTAAAGCTGAGGAGT | 5684 |
| Osr1 | GGAGCTTTAGAATGGAATTC | 5685 |
| Osr1 | GAAACTAGTGGATGGAGGGC | 5686 |
| Osr1 | GTATGCCTAAGGTGCTGGTG | 5687 |
| Osr1 | GGAGTTTCCTCTTCTTCACT | 5688 |
| Osr1 | GGGACTGAGGTCACCTCAGT | 5689 |
| Osr1 | GCTTGCTTCCAGATGCATCC | 5690 |
| Osr2 | GTCACCTTGGGCTCTGTGTC | 5691 |
| Osr2 | GGTCTGTCTACCTGGTGAGC | 5692 |
| Osr2 | GAGAACGCCTAGGAAGAGTT | 5693 |
| Osr2 | GGACTTCTCTGCGGGCTTGG | 5694 |
| Osr2 | GAGCTGTCCCAGCTCCCTGT | 5695 |
| Osr2 | GACACGGAGCTGAGGGTGGA | 5696 |
| Osr2 | GGTCTTGAGCCTCAGCTCCC | 5697 |
| Osr2 | GGACACCACGGCTCGTTTGG | 5698 |
| Osr2 | GGCAGGTTATTGTTTGCCAA | 5699 |
| Otp | GCAGAGCGAGAACAGGGAGT | 5700 |
| Otp | GTTTGGGAGCAGATCAGAAA | 5701 |
| Otp | GGCTCAGTAGGCCAGAGTCT | 5702 |
| Otp | GGGAAATCTGAGAATGGGAG | 5703 |
| Otp | GGGCTCAGTAGGCCAGAGTC | 5704 |
| Otp | GAAGAGCAAGGAGACAAAGG | 5705 |
| Otp | GGTGGGATGTGTGTGGGCTG | 5706 |
| Otp | GGAAATCTGAGAATGGGAGG | 5707 |
| Otp | GTGCTTGTGCTCTGAAGTCT | 5708 |
| Otp | GAGCAAGGAGACAAAGGAGG | 5709 |
| Otx1 | GTTTATTCGGCTGGAAACTG | 5710 |
| Otx1 | GAGGATGGAGGAGTTTGTGG | 5711 |
| Otxl | GGTGAGAACGGCAGAAGATG | 5712 |
| Otx1 | GGCACTCCTTGATGCTCCCT | 5713 |
| Otx1 | GAGCGGAGGAGGAGTTGCTG | 5714 |
| Otx1 | GACAAAGGATCAGGGCCGCC | 5715 |
| Otx1 | GGCATCTCCTACTGAATACT | 5716 |
| Otx1 | GAACGAGTTGAGGAGGGCGA | 5717 |
| Otx1 | GAGGAGTGGCGTCAAGCTGC | 5718 |
| Otx1 | GCTAGGBAAAGGTACGGGTC | 5719 |
| Otx2 | GAGCCGGAGGGAAAGGAAGA | 5720 |
| Otx2 | GCCTGTATTAACATCGATGG | 5721 |
| Oxt2 | GCAAAGAATGTGTGGGTCAG | 5722 |
| Oxt2 | GCCCTAGCGGCTTTGAGAAG | 5723 |
| Oxt2 | GAAAGGAAGAAGGAGGTACG | 5724 |
| Otx2 | GGACAGCCAGACCTGAGGGT | 5725 |
| Oxt2 | GCTAATTGCTGTAAATCCCA | 5726 |
| Oxt2 | GAGCGTGCATTTGGAGGCGT | 5727 |
| Oxt2 | GTCCCTAAGGCCTTTCATTT | 5728 |
| Ovol1 | GGGATGGAACCTGCAGTTAA | 5729 |
| Oval1 | GGAATGGAAACCGGTTCGAC | 5730 |
| Ovol1 | GAATTGCTCACCCTGGCCTT | 5731 |
| Ovol1 | GAGAGGTATTTGCTGGGCAC | 5732 |
| Ovol1 | GCCTGGGTTAGGTTGGACTC | 5733 |
| Ovol1 | GGTGCTGGCCTGGGTTAGGT | 5734 |
| Ovol1 | GTCCAAATCAGAGTGAAAGG | 5735 |
| Ovol1 | GACGATTCATCCCATGAACA | 5736 |
| Ovol1 | GAAGTGTGGGCTATGACAGA | 5737 |
| Ovol2 | GGAGGGAAGTGTGTGTGTGT | 5738 |
| Ovol2 | GCGAGAGGAAACTTGGCGCG | 5739 |
| Ovol2 | GTGCAGGAAGAGGGCAGGCT | 5740 |
| Ovol2 | GGCCAAAGTGGCCTGGAAGT | 5741 |
| Ovol2 | GTTGACACCGTTATGTTGCA | 5742 |
| Ovol2 | GGGTTCCTACAACGATAGCT | 5743 |
| Ovol2 | GGAATCTCGGTGGTCGGATG | 5744 |
| Ovol2 | GGGCTAAATTGCCTGGCGGC | 5745 |
| Ovol2 | GACCTTCAGGAGCGGTTTAG | 5746 |
| Ovol2 | GGGATCCTCTCTTCCGACTG | 5747 |
| Parp1 | GATTACTGATGCCTAGCGGC | 5748 |
| Parp1 | GCACGTGTCCTTGGGAGGTT | 5749 |
| Parp1 | GAAATTTAACAAATGGCGTG | 5750 |
| Parp1 | GTGGGTGAGGTGGAATTATT | 5751 |
| Parp1 | GGGCTTCAFCACGTGTCCTT | 5752 |
| Parp1 | GCTGGTCTCTGCCTCTGGGA | 5753 |
| Parp1 | GATAGATTACTGATGCCTAG | 5754 |
| Parp1 | GGGCAAGCTGCAGCTGTTTG | 5755 |
| Patz1 | GGCGTTCGGCACAAAGAAAG | 5756 |
| Patz1 | GGTAGACCTAGAGAGGGAGG | 5757 |
| Patz1 | GTCAAGGATGGTCCAGAAGA | 5758 |
| Patz1 | GTGTTCAACTGTGCTATTGA | 5759 |
| Patz1 | GAAACTGTGTACCTCAGTCT | 5760 |
| Patz1 | GGTATTACCATGCAAATGAA | 5761 |
| Patz1 | GATGCGCACGTGCTGAGTCG | 5762 |
| Patz1 | GGATCAGGCTGCGCTAGCAT | 5763 |
| Patz1 | GAAGGTAGACCTAGAGAGGG | 5764 |
| Patz1 | GTATTACCATGCAAATGAAC | 5765 |
| Pax1 | GACCAGCTCATTGCTTCCTT | 5766 |
| Pax1 | GGGCCAGAGGACTAAGGTGA | 5767 |
| Pax1 | GGAGAGGAATGAGTTCTGGT | 5768 |
| Pax1 | GAAACCACTACTCGCTCACT | 5769 |
| Pax1 | GTTCCTGGCTCAGGTATCCC | 5770 |
| Pax1 | GGGTAAGAGAAAGGCGGAGG | 5771 |
| Pax1 | GGAAGCCAAGAACTAGGAGG | 5772 |
| Pax1 | GGCACTCCTGTTATGAGTAC | 5773 |
| Pax1 | GGAAGGCTGCCCTGTTCTAA | 5774 |
| Pax2 | GAGAATCATGCGTGCGTGGA | 5775 |
| Pax2 | GGTAGGAGTCAGTCTGAGAC | 5776 |
| Pax2 | GAAGCCCTTTGTCTCCTAAC | 5777 |
| Pax2 | GTATAAGTCACATGCGGCTT | 5778 |
| Pax2 | GAGTTTGAGAGGCGACACGG | 5779 |
| Pax2 | GCTGGCGAATCACAGAGTGG | 5780 |
| Pax2 | GAACCAAGAGAGCTCAGGGC | 5781 |
| Pax2 | GGCGGTTCTAAATGCCCGGT | 5782 |
| Pax2 | GCATGCCTTCTCAGGACTCC | 5783 |
| Pax2 | GGCCAGCCTAATGAATATTC | 5784 |
| Pax3 | GGCTCCAAGTTGCAAGCAAT | 5785 |
| Pax3 | GGCAAAGACACGCGCTGATT | 5786 |
| Pax3 | GGGCAGACAGAGGAAATAAG | 5787 |
| Pax3 | GGCTTGGGATCCTGCACTCA | 5788 |
| Pax3 | GAACTGGAGAGTGCTAGGTA | 5789 |
| Pax3 | GCAACAAGTAGGGATGAAGA | 5790 |
| Pax3 | GACTTCCCTGGACACATGTG | 5791 |
| Pax3 | GCAGACCACACATGTGTCCA | 5792 |
| Pax4 | GTCTGGGTAGGGTAGGGTGC | 5793 |
| Pax4 | GGAGCTGGAATGGCCTTGGC | 5794 |
| Pax4 | GGGCTTCAGAGTCCACCTTG | 5795 |
| Pax4 | GGGTATTCAACATGAACACC | 5796 |
| Pax4 | GGATCGTTGGCTCCTGCCTT | 5797 |
| Pax4 | GAGCCTTCACTCAGGAGCAG | 5798 |
| Pax4 | GTATAATTGTGAGCAGATGG | 5799 |
| Pax4 | GCTTCACAGAGCCTTCACTC | 5800 |
| Pax4 | GTTGTAAAGACTGAGAGAGA | 5801 |
| Pax5 | GCAGTGTGTCAGGCCCAGAC | 5802 |
| Pax5 | GGCAGAAACGAAATAGTGAT | 5803 |
| Pax5 | GAAGGAGAACCTGAGTCACA | 5804 |
| Pax5 | GAGGCGGCATTGCTGCTCTC | 5805 |
| Pax5 | GGAAGGAGAACCTGAGTCAC | 5806 |
| Pax5 | GCAAAGGGCTGCAGAAGGGT | 5807 |
| Pax5 | GCTCTAGGGCCACTGGACAA | 5808 |
| Pax5 | GGTGTAGGAGAAAGCAGAAA | 5809 |
| Pax5 | GCCTAGAGTTCGAGGATAGG | 5810 |
| Pax6 | GAACCTAAGGACAGGCTACG | 5811 |
| Pax6 | GAGGACCTAAGGCACTGGAT | 5812 |
| Pax6 | GCTAATGGGCCAGTGAGGAG | 5813 |
| Pax6 | GTATTGTCCTCCCTGAGGTT | 5814 |
| Pax6 | GAGGGCTGCTGGAGCTTGTT | 5815 |
| Pax6 | GGGAAAGGTGGCTGACTAGC | 5816 |
| Pax6 | GACAGATAGATAAGCTGGCG | 5817 |
| Pax6 | GAAGAGTCTAAGGCAATGAA | 5818 |
| Pax6 | GACTTCTCTGATCTGGAACT | 5819 |
| Pax6 | GAAGTTCTGACTGGAAGGTA | 5820 |
| Pax7 | GAGGACCAGACCACAGAGTC | 5821 |
| Pax7 | GGGTCAGTAGCATGCTGAGA | 5822 |
| Pax7 | GGGAGACAGACAGATAAAGC | 5823 |
| Pax7 | GTCCACTAGAAAGGGCTCCA | 5824 |
| Pax7 | GTGGACTCAGAATCTCCTGG | 5825 |
| Pax7 | GCAGCTATGTGTCCTGTGCA | 5826 |
| Pax7 | GCGCTGAGAGGAGGGATCGA | 5827 |
| Pax7 | GAGGCGACATCAGCAAGGCT | 5828 |
| Pax7 | GGCGATTTCACATCCAGGAG | 5829 |
| Pax7 | GGGATCTTCTCTGTCCGCTC | 5830 |
| Pax8 | GGAGAATCTACAGGCCAGTG | 5831 |
| Pax8 | GAGTGAGAACTTTGGTCTGA | 5832 |
| Pax8 | GATGCTGCATTTAGGTACCT | 5833 |
| Pax8 | GAGCTTCTGTTTCTGGAGGA | 5834 |
| Pax8 | GAGGGACTGTTCTGCCTTGA | 5835 |
| Pax8 | GGAGATAGGTTGTTAGCTTG | 5836 |
| Pax8 | GGTTGCCTGCACAATCTTCC | 5837 |
| Pax8 | GTATGTGGGAGCAAATGTCC | 5838 |
| Pax8 | GAGTGGGAGATGAAAGCCTG | 5839 |
| Pax9 | GCCATGTCATCTCCAAGAAG | 5840 |
| Pax9 | GGCACTGTGTGCCCTTGCTT | 5841 |
| Pax9 | GGGCTAAACCTCAGTCAAGC | 5842 |
| Pax9 | GTTCGTGCCCATCCAAAGCT | 5843 |
| Pax9 | GGAGGTGTGCGACAGCTAAA | 5844 |
| Pax9 | GGTCTCCTCTGGACAATTAC | 5845 |
| Pax9 | GGAGGGTCAGAGCAAACAGC | 5846 |
| Pax9 | GCTAAGACTCCTGGGATCTG | 5847 |
| Pax9 | GAATTGAGTGCGGGTTACTG | 5848 |
| Pax9 | GTGGAATCTAGCTCTTCCCA | 5849 |
| Pbx1 | GCAAACATTTGGCAAGATAA | 5850 |
| Pbx1 | GTAAGCGCAAGTGGGCTTTG | 5851 |
| Pbx1 | GAATCTCATAAAGTGTTGCC | 5852 |
| Pbx1 | GGTGTGAGGGAGAAAGATAA | 5853 |
| Pbx1 | GAGATCACTCTGGCCCGGAG | 5854 |
| Pbx1 | GATTTATGTCTTGGCCCACA | 5855 |
| Pbx1 | GCTGCACAAATCGTCTGAGA | 5856 |
| Pbx1 | GGCCAGAGTGATCTCAAAGG | 5857 |
| Pbx1 | GCCAGGTCAAGTATTAAGAG | 5858 |
| Pbx1 | GCAATAGAAACCGGAAGGCA | 5859 |
| Pbx2 | GGACTGAAGACTGGTATCTG | 5860 |
| Pbx2 | GAGGGAGGAGCAGATCTCCA | 5861 |
| Pbx2 | GCTCTCAGCGATCTGGCCAG | 5862 |
| Pbx2 | GACATGTGGGACCTAGTGAC | 8863 |
| Pbx2 | GTCAAACAAGCTGTTTGGGT | 5864 |
| Pbx2 | GACAGACTCAGCAGCAGGGT | 5865 |
| Pbx2 | GGGTCAAGATCTTCCAGGGT | 5866 |
| Pbx2 | GGGAACCAACCCAGGGAGAA | 5867 |
| Pbx2 | GAGAGGAGGGAGGAGGGAGA | 5868 |
| Pbx2 | GAGTTGCTGCTGAGAGTTCA | 5869 |
| Pbx3 | GGCGGGACAGAGCCAAGAAA | 5870 |
| Pbx3 | GACTTTCGAACGCTCCAATT | 5871 |
| Pbx3 | GGTAAGCCTTGTAGCTTGCC | 5872 |
| Pbx3 | GTCCACAGCGGCTGCTGACT | 5873 |
| Pbx3 | GTGAAGTTGACTACAGCCGA | 5874 |
| Pbx3 | GCGCAAAGCCCAATGAGAGA | 5875 |
| Pbx3 | GTCAAGATTATGAGCCTAGA | 5876 |
| Pbx3 | GCTGTCCTCAGTCTCCTCCG | 5877 |
| Pbx3 | GCAGTTCTTTAAATGCCAGA | 5878 |
| Pbx3 | GCTAATAATAGGGAAGGAGC | 5879 |
| Pcbp1 | GAACTGCTCGCTTGCTCCCT | 5880 |
| Pcbp1 | GCGCCTTGTGCTTTCTTTGC | 5881 |
| Pcbp1 | GGACCCGGCAGAGATTGAGA | 5882 |
| Pcbp1 | GACCTCCTCCAGGCTGACTA | 5883 |
| Pcbp1 | GCACCGCTCCTCTAAGGTCT | 5884 |
| Pcbp1 | GAGCCGGCAAAGAAAGCACA | 5885 |
| Pcbp1 | GTCAGGGCGACCTCCCAAGA | 5886 |
| Pcbp1 | GGAGCCAGGTGGAGGGAAAT | 5887 |
| Pcbp1 | GGGACCCGGCAGAGATTGAG | 5888 |
| Pcbp2 | GCTCAGGCCTAAATACGAAG | 5889 |
| Pcbp2 | GGATGACAAAGGTGAACCTC | 5890 |
| Pcbp2 | GCTTTGGGATCAAGATAGGA | 5891 |
| Pcbp2 | GTTCTTCCGCTAGAGGCCAC | 5892 |
| Pcbp2 | GTGATACAAGCTGATACCAT | 5893 |
| Pcbp2 | GAAGAGGTGGCCAATGCTTT | 5894 |
| Pcbp2 | GGCCAAAGGTAAAGGGTAAA | 5895 |
| Pcbp2 | GTGGTGGAAAGAAAGACATT | 5896 |
| Pcbp2 | GTGGCCTCTAGCGGAAGAAC | 5897 |
| Pcbp2 | GAAAGACATTTGGAGTCACT | 5898 |
| Pcbp3 | GATGCTTCTCGAAAGTCTGG | 5899 |
| Pcbp3 | GAGTGGCTGGTTGCACGGAG | 5900 |
| Pcbp3 | GGTATCTAGGTACTGATGGA | 5901 |
| Pcbp3 | GGAAGACACACTTAGTCATT | 5902 |
| Pcbp3 | GAAGGCAGGAAGCCTGCATT | 5903 |
| Pcbp3 | GGCAACCTGTAATCTGGAGG | 5904 |
| Pcbp3 | GGCCTCTGCTGACCTTCAGC | 5905 |
| Pcbp3 | GTCCAGCTGAAGGTCAGCAG | 5906 |
| Pcbp3 | GCTACTTGGTTACTATGGTG | 5907 |
| Pcbp3 | GGTGGTTCTGATGGTCGGGC | 5908 |
| Pcbp4 | GAAAGCTGGAGGAGCCCATG | 5909 |
| Pcbp4 | GAGCCTGTAGAGGAAGCTTA | 5910 |
| Pcbp4 | GGTTACAGACTGCCTGCTCT | 5911 |
| Pcbp4 | GGTTACAGACTGCCTGCTCT | 5912 |
| Pcbp4 | GATCTCTTGCTGTCTTCTCC | 5913 |
| Pcbp4 | GAGCCATACAGGGAGGATCC | 5914 |
| Pcbp4 | GATCCGTGCTTGATTACCTG | 5915 |
| Pcbp4 | GCCGCGCTGTAATCGGATCC | 5916 |
| Pcbp4 | GGAGAGACAACGGCAATGAA | 5917 |
| Pcbp4 | GCAGTTTCCTGAAGGATGGA | 5918 |
| Pdx1 | GTGGTTGAGCAGTTGGGCAA | 5919 |
| Pdx1 | GAAATGCGTATCACCCATAA | 5920 |
| Pdx1 | GAGCTGCTGTTAAATGGCTC | 5921 |
| Pdx1 | GAGTGTCTCTGATTTCTTCA | 5922 |
| Pdx1 | GCCTCTGACCTGGTCCTCCA | 5923 |
| Pdx1 | GGAGGACTGCCTTAAGAAGG | 5924 |
| Pdx1 | GGTGTTCGGTAGCAACCAAG | 5925 |
| Pdx1 | GCGAGACTTGGGACAAAGAT | 5926 |
| Pdx1 | GAAATTCCACTAAAGACGCC | 5927 |
| Pgr | GTCATGAGAACACTGTGGAG | 5928 |
| Pgr | GCAGATCATCGGTTACTGTG | 5929 |
| Pgr | GGTAGTAATGTTGCAAAGAA | 5930 |
| Pgr | GCAGGAGAACGAGTAAGAAT | 5931 |
| Pgr | GAGAAATGGCTGATTCTAGG | 5932 |
| Pgr | GCTGGGATTGTAGAGAACCT | 5933 |
| Pgr | GTGTGGGAAGCAAAGAAATG | 5934 |
| Pgr | GATCTAGCCAGTGATTGGCT | 5935 |
| Pgr | GCCATAGAGACTGTCGCTGC | 5936 |
| Phox2a | GGGACAGGATAAGGGACTCT | 5937 |
| Phox2a | GGATAAGGGACTCTCGGATT | 5938 |
| Phox2a | GTCACAAATCCCAGCTCTCG | 5939 |
| Phox2a | GCATCTTGTGTAGAGATCGA | 5940 |
| Phox2a | GAGGTCACAAGCTATTGGAG | 5941 |
| Phox2a | GACAACAGAGCTGAGAAGCC | 5942 |
| Phox2a | GACAGGGATGAGAAAGAGAC | 5943 |
| Phox2a | GCCAGGTCATTGACCCTCCA | 5944 |
| Phox2b | GTAGGGTGGCAGTGGAGAGC | 5945 |
| Phox2b | GCTGCGATGAAAGGCTTGTG | 5946 |
| Phox2b | GCTGGTTAGAAGGGAGGATC | 5947 |
| Phox2b | GCCCTGTAACATAGCGTAAG | 5948 |
| Phox2b | GCAGCTTGGAGCCAGACTAC | 5949 |
| Phox2b | GCAGGAAATGCCCTCGGAGG | 5950 |
| Phox2b | GAAAGAGAGTGCGAGAGCAA | 5951 |
| Phox2b | GAACACAGTGTAGAAATTCG | 5952 |
| Phox2b | GGCCTCAGCCCTAACTCCCA | 5953 |
| Phox2b | GGTACATTTCGTGCTGGGCT | 5954 |
| Pitx1 | GGAAAGCTACAATCTTTCTG | 5955 |
| Pitx1 | GGTAACTTTGCTCCAGTGTG | 5956 |
| Pitx1 | GACTGATGTCTCACAACCCT | 5957 |
| Pitx1 | GGAATCACGGATGGCCCTGG | 5958 |
| Pitx1 | GGATCTATGAGGATAGTGGA | 5959 |
| Pitx1 | GTGGTAGAGAAGGTAGGAGA | 5960 |
| Pitx1 | GAGCAAAGTTACCCTAAAGG | 5961 |
| Pitx1 | GCCGAGTGGAGGACTCTCAT | 5962 |
| Pitx1 | GACCACTCTTGTGAGCCTAG | 5963 |
| Pitx1 | GTCGCTTCTCCCAGGAACTC | 5964 |
| Pitx2 | GTGGGAGAGCCACAGCCGAT | 5965 |
| Pitx2 | GGGCACAGCAAGGAATTAGT | 5966 |
| Pitx2 | GGGATGGTAGGAAAGGGACA | 5967 |
| Pitx2 | GTTCAGGAAGTATTCCGGTG | 5968 |
| Pitx2 | GTGAGCTAGCGGCAGAAGGT | 5969 |
| Pitx2 | GCATTGTGGGAGATGGCACT | 5970 |
| Pitx2 | GAGGTTTGCAGCCAGGGCTG | 5971 |
| Pitx2 | GAGAAATGCAGTTTAATGGC | 5972 |
| Pitx2 | GTCATTGGCTGGCAAGTGCC | 5973 |
| Pitx2 | GTCTGGGTGGCTTACGAGGA | 5974 |
| Pitx3 | GCATAGACACAGGGAGTTGT | 5975 |
| Pitx3 | GAAAGACAGACAGCGACAAG | 5976 |
| Pitx3 | GACAACAGATTTGGTCCTGT | 5977 |
| Pitx3 | GGAGTCACGAGAAGCAGTGC | 5978 |
| Pitx3 | GATGCCTCCCTGGCTTCCAG | 5979 |
| Pitx3 | GAAAGATAGGATGGAAAGGA | 5980 |
| Pitx3 | GGACAGAGAACCCGGCTGTC | 5981 |
| Pitx3 | GTCAGGCGGGACAGAGAACC | 5982 |
| Pitx3 | GAGGCATACCTAGATAGGGA | 5983 |
| Pknox1 | GTACGCTGCTGCAGATGATC | 5984 |
| Pknox1 | GGAAGAACAGAGCCGTGCAC | 5985 |
| Pknox1 | GTCAGACCGCAGTCACTTCA | 5985 |
| Pknox1 | GCCCTCCAGCAATGTAGTGG | 5987 |
| Pknox1 | GCCCTGGGACACCAAAGCAC | 5988 |
| Pknox1 | GAGCTGTCTGTACTAGGAGG | 5989 |
| Pknox1 | GAGCCGGGACTGGAATCACT | 5990 |
| Pknox1 | GTGCCAAATGACCACAAACT | 5991 |
| Pknox1 | GCGCCTCGCAATCAAGGTTC | 5992 |
| Pknox1 | GAGAGGTCTGACTAGTGAAG | 5993 |
| Pknox1 | GTGGAGTTGCTGGCAGGACC | 5994 |
| Pknox2 | GCTGGGCACCGTAAGGTGAG | 5995 |
| Pknox2 | GCCTGAAACCGCTTCTCAAG | 5996 |
| Pknox2 | GATTCTCTGGTCCTCTGTGT | 5997 |
| Pknox2 | GTTGAGAAGCAGGGTGGACA | 5998 |
| Pknox2 | GGTGGACAGACCCAAAGGGC | 5999 |
| Pknox2 | GAGCACACACATATTTGTGA | 6000 |
| Pknox2 | GGAGTTCTTAAGATTCTCCT | 6001 |
| Pknox2 | GCTAGAGTGTCTCCCTCTAC | 6002 |
| Plagl1 | GGTTTCAAGGCATGGAGGCC | 6003 |
| Plagl1 | GGTTGGGAGAGCAGGCTGTT | 6004 |
| Plagl1 | GTGACAAATCGCAGATGCCG | 6005 |
| Plagl1 | GTGAATCACTAAGATCGGTG | 6006 |
| Plagl1 | GCTCGTCAACAGGGAGAATA | 6007 |
| Plagl1 | GACACTGTAAGAATGCCGTT | 6008 |
| Plagl1 | GTTACAGGGTTTCTGCCTGT | 6009 |
| Plagl1 | GTTTGCGATGTGGCCGAGGG | 6010 |
| Plagl1 | GGGAGAATAAGGTTTCTACA | 6011 |
| Plxna2 | GAAAGGTTGTACCAGGGTCT | 6012 |
| Plxna2 | GTTGGCTTTCTAGAATTTGT | 6013 |
| Plxna2 | GGAGTCTGGCTTTGCATCTC | 6014 |
| Plxna2 | GGGTCACCTAGGAAAGACAA | 6015 |
| Plxna2 | GGTCTAGGGACTCATGTCTT | 6016 |
| Plxna2 | GCCTGGTAAGCCAGCTGGCT | 6017 |
| Plxna2 | GACAGATCACACTGCAGGCT | 6018 |
| Plxna2 | GAAGACCCTGAGACCTTGCA | 6019 |
| Plxna2 | GTAATATAGGAGGCCGGCTG | 6020 |
| Plxna2 | GGGAGGCTTTGCTTGGTGAC | 6021 |
| Pml | GGAACAGAGAATAGGCACAT | 6022 |
| Pml | GGCCTATGAGAAGTAACTGT | 6023 |
| Pml | GTGGACAAGTTGAAGTCAGA | 6024 |
| Pml | GGGAACTTAGAAATGAGCTA | 6025 |
| Pml | GTCCCACAGTTACTTCTCAT | 6026 |
| Pml | GGTAGACAACAGGGAGGCAA | 6027 |
| Pml | GTCTTTCTCTTAACTTTGGA | 6028 |
| Pml | GCCAGTTTGGGAAGTAGTCA | 6029 |
| Pou1f1 | GGTGAAATATGACAATGCAT | 6030 |
| Pou1f1 | GACACTTAGGAAAGCATTGG | 6031 |
| Pou1f1 | GACAAAGCAACACTCCTGTG | 6032 |
| Pou1f1 | GTTGACACTTAGGAAAGCAT | 6033 |
| Pou1f1 | GGTACGTCCCTTACAGAGAT | 6034 |
| Pou1f1 | GTAGAGCTCCCATCTCTGTA | 6035 |
| Pou1f1 | GTAAGTAGAAATAAAGGGAT | 6036 |
| Pou1f1 | GTGTCTTCTCTGCGTATTCC | 6037 |
| Pou2f1 | GGTAGGAGGATGTGATGACG | 6038 |
| Pou2f1 | GGGTAGTCCAGAGTCCTTGC | 6039 |
| Pou2f1 | GTGTTCCCTTAATACATGAC | 6040 |
| Pou2f1 | GTTACACATGATGTAAACAA | 6041 |
| Pou2f1 | GCAATCTTCTCTCAGATGTG | 6042 |
| Pou2f1 | GTGAGGCTTGAAGAGAGGGC | 6043 |
| Pou2f1 | GGAGAGAGTACCAGCAGGTG | 6044 |
| Pou2f1 | ACTTTAGTGTCTCAGCTCTG | 6045 |
| Pou2f1 | GGGTTGGGTTTCTCTTCAGA | 6046 |
| Pou2f1 | GAATGTGCATCGTCCTCAAA | 6047 |
| Pou2f1 | GGTAAGAATAAATAGGTCCT | 6048 |
| Pou2f1 | GACTGGACAAGTTCTACTAT | 6049 |
| Pou2f1 | GTTCAAGTCACTGAACCTGG | 6050 |
| Pou2f1 | GGTAGGGATCTGTTTATTTA | 6051 |
| Pou2f1 | GGTCCAAGATGCCAGGCCAT | 6052 |
| Pou2f1 | GAATGAATAGTGTATCCACC | 6053 |
| Pou2f1 | GCATTTCCGTGGCCCATTTG | 6054 |
| Pou2f1 | GTGCCAGCATAATAATTAGG | 6055 |
| Pou2f2 | GAGCTGAGTTCGTTCCTGTC | 6056 |
| Pou2f2 | GATCCGGCGAGAAATATGAG | 6057 |
| Pou2f2 | GTGCCACAGGTAAGGCATCC | 6058 |
| Pou2f2 | GGAGTTGAGAGAGATGAACT | 6059 |
| Pou2f2 | GCTCGCTGAAAGCGCTCCAC | 6060 |
| Pou2f2 | GCAGCGTCCAGTCAATGGGC | 6061 |
| Pou2f2 | GGCACATGGCTTAGATGGTG | 6062 |
| Pou2f2 | GCCACTGTGCCACTGCTCAT | 6063 |
| Pou2f2 | GTAATTAAAGGAGACGCAGG | 6064 |
| Pou2f2 | GACAGCTAGAAGCCTGTCAG | 6065 |
| Pou2f3 | GAACTCTGCAGCCTATGTGG | 6066 |
| Pou2f3 | GACCATCCTCGGAAATGACT | 6067 |
| Pou2f3 | GAGATACGGAAATCACAGAT | 6068 |
| Pou2f3 | GGACCAGAGGTGATTGATGG | 6069 |
| Pou2f3 | GTCATTTCCGAGGATGGTCT | 6070 |
| Pou2f3 | GGACAGGTGTTCCTCAGGGT | 6071 |
| Pou2f3 | GATTGATGGTGGGAACCGGC | 6072 |
| Pou2f3 | GACAGGTGTTCCTCAGGGTC | 6073 |
| Pou2f3 | GTCCCTTCCTTCTTGCCAGG | 6074 |
| Pou3f1 | GTTGTGGCGCTGAGTCTAGA | 6075 |
| Pou3f1 | GGTCGCATCGCGTTCTCCCA | 6076 |
| Pou3f1 | GCTAATGACATCATCCTCAT | 6077 |
| Pou3f1 | GGTTACGCTGTACAGATTTG | 6078 |
| Pou3f1 | GATTTCTGGGTGCCGAGCTG | 6079 |
| Pou3f1 | GACTCTCAGACTGTCAAACT | 6080 |
| Pou3f1 | GCAGCAGCATGAACTAGGAA | 6081 |
| Pou3f1 | GTTCTCTCATTCCAGAACCC | 6082 |
| Pou3f1 | GACTCAGCGCCACAACTAGC | 6083 |
| Pou3f1 | GGCAACTAACTTTCCTCAGT | 6084 |
| Pou3f2 | GAGGAAGGACTGAGAAGACT | 6085 |
| Pou3f2 | GTGTAAGGGATCTTTGTTAC | 6086 |
| Pou3f2 | GTCTAGCTGTGTGTGTGTGT | 6087 |
| Pou3f2 | GGATGGTGGAAGAGAAAGAC | 6088 |
| Pou3f2 | GAGGCTTGGAAGACTTGTAT | 6089 |
| Pou3f2 | GCCACTTAGGCTGCGCCTTT | 6090 |
| Pou3f2 | GTTTCCAGATTTCTTTCCGA | 6091 |
| Pou3f2 | GGCGTGGACATTTCACAACC | 6092 |
| Pou3f2 | GTCTACTTTCTCTTCCCAAT | 6093 |
| Pou3f2 | GTTTATGAAAGTGTATGGAG | 6094 |
| Pou3f3 | GCGTGCCCTTGTGAGTTTGG | 6095 |
| Pou3f3 | GCCAAGGGAACGCAGACGTT | 6096 |
| Pou3f3 | GAAGCGGTTCCTTTCTTCCT | 6097 |
| Pou3f3 | GCTGCAGGTTTCCCAGATGA | 6098 |
| Pou3f3 | GCGGCTGCACAAAGTTGCAG | 6099 |
| Pou3f3 | GAAGAGTGCATTGGTGGAGG | 6100 |
| Pou3f3 | GGCACCCTCCAAACICACAA | 6101 |
| Pou3f3 | GCTTGCTATTCTTGGGCATG | 6102 |
| Pou3f3 | GCAATCTGGCCGCTCCTAGT | 6103 |
| Pou3f4 | GAGAGACCTCTGATGGAAAC | 6104 |
| Pou3f4 | GGATAACGTTTATTGGATCA | 6105 |
| Pou3f4 | GCAAATATAATTACACAGCT | 6106 |
| Pou3f4 | GAGGTCTCTCCACTATGCCA | 6107 |
| Pou3f4 | GATGCTCCTTAGCTATATAA | 6108 |
| Pou3f4 | GCCACCAAGATCTCTCTCAC | 6109 |
| Pou3f4 | GACCTCTGATGGAAACTGGC | 6110 |
| Pou3f4 | GGGAACCAAAGCCGCTAGCG | 6111 |
| Pou4f1 | GCCTTTCTATCTGTCATTTA | 6112 |
| Pou4f1 | GTCTGATTTCTGGGAGTTGA | 6113 |
| Pou4f1 | GCGGGAAATGGACTATGAGC | 6114 |
| Pou4f1 | GTTCATTTGCTGGTGCAAGA | 6115 |
| Pou4f1 | GCAGGTGATGCACCTGTAGC | 6116 |
| Pou4f1 | GCCCGTTATTGTTGAAGGGT | 6117 |
| Pou4f1 | GAGGGTGTCCAGCCTTCAGC | 6118 |
| Pou4f1 | GCTTGTTAGGCACCGGGTAG | 6119 |
| Pou4f1 | GCAGATCAAGGGCCTCTCTG | 6120 |
| Pou4f1 | GGGCAACTGCACCCTGTGTC | 6121 |
| Pou4f2 | GTGCCTAGTGCAGAAAGACG | 6122 |
| Pou4f2 | GCTAGTGCAAACCACTTTCC | 6123 |
| Pou4f2 | GAGCAACTGACAGGACGAGA | 6124 |
| Pou4f2 | GACTTGCCCAAACACTATTG | 6125 |
| Pou4f2 | GACTGTTCAAGTAAGTGCTT | 6126 |
| Pou4f2 | GGAGAGCAGGTGGCCAGGTA | 6127 |
| Pou4f2 | GGTCCCGTGGCAGAGAGAGA | 6128 |
| Pou4f2 | GAGAGGAAATAGGTTGTGTT | 6129 |
| Pou4f2 | GACCAGCAAGACACTGGCAA | 6130 |
| Pou4f2 | GTCCTTGCCCAGGCTTCTTA | 6131 |
| Pou4f3 | GCTTTCTTGGGTCTTGCTGG | 6132 |
| Pou4f3 | GGGAAGTAGTGGTATTGTCC | 6133 |
| Pou4f3 | GTGGCACAAAGCCAGTAATA | 6134 |
| Pou4f3 | GCGCCACTCTGAGCCTGATG | 6135 |
| Pou4f3 | GGAAGCGGGAATAAACATGG | 6136 |
| Pou4f3 | GGGTATAAATGCTGTGGAGG | 6137 |
| Pou4f3 | GAAGAGCCCAAAGTCAGACA | 6138 |
| Pou4f3 | GCTCGAGCTGCCTGGATGAA | 6139 |
| Pou4f3 | GGCAGTCCTGAGGAAAGAGG | 6140 |
| Pou4f3 | GACCCTTAAGAGGCTCCATG | 6141 |
| Pou5f1 | GACCCATGTGGTAGAAGGAG | 6142 |
| Pou5f1 | GGATGGCTGAGTGGGCTGTA | 6143 |
| Pou5f1 | GAAACCGGCCTGGATTGTTT | 6144 |
| Pou5f1 | GTGCAATGGCTGTCTTGTCC | 6145 |
| Pou5f1 | GACTTTGCAGCCTGAGGGCC | 6146 |
| Pou5f1 | GTCTGGACAGGACAACCCTT | 6147 |
| Pou5f1 | GCTTCCTCAATAGCAGATTA | 6148 |
| Pou5f1 | GAGTGCCTGTCTGCAAGGGA | 6149 |
| Pou5f1 | GAAGCCTGGGATGAGGAGGT | 6150 |
| Pou5f1 | GTGTCTTCCAGACGGAGGTT | 6151 |
| Pou5f1 | GCTTCCACTGGAGACGTTTA | 6152 |
| Pou6f1 | GGCCAGACAGACAGGTAGGC | 6153 |
| Pou6f1 | GTGCTGCTTGACTAGCCCGC | 6154 |
| Pou6f1 | GACAGACGTGAACTTGAGGT | 6155 |
| Pou6f1 | GGAGACTCAGAGGCCGATTA | 6156 |
| Pou6f1 | GCGAAGAGGGAAACACTGTT | 6157 |
| Pou6f1 | GGGATCTTGCTCTGCCCTGG | 6158 |
| Pou6f1 | GTCAAGTGGCTGGGCAGCAG | 6159 |
| Pou6f1 | GTGAGTCTGTTGTGAATGAA | 6160 |
| Pou6f1 | GGGAGACTCAGAGGCCGATT | 6161 |
| POU6F1 | GAAATCTCGTTTGCTCTTGC | 6162 |
| POU6F1 | GCAGGCTGGACAGTGATCTG | 6163 |
| POU6F1 | GCCGTCGCTTGCTTTAGTCT | 6164 |
| POU6F1 | GACAATCCCTACAGCAACTG | 6165 |
| POU6F1 | GGGGCAGCCACATTGCTGTA | 6166 |
| POU6F1 | GCATAAGGAGAAGACCTCAA | 6167 |
| POU6F1 | GATGCTTGCTTGTGCTCAGT | 6168 |
| POU6F1 | GCCAGCCCTTCAAGAATCAA | 6169 |
| POU6F1 | GGGAAAGGGAGTACAGTCAA | 6170 |
| Ppara | GAGTCCCAGGATGAGAAATG | 6171 |
| Ppara | GGAAGGAAGGTGTAACGTGG | 6172 |
| Ppara | GGTGAGTGCCAGTCCTAGGA | 6173 |
| Ppara | GACAGTGAGGTGGGTGGACA | 6174 |
| Ppara | GATGTCACCTGCAAATGTGT | 6175 |
| Ppara | GTCTGGGTTGAGCTTTCCTC | 6176 |
| Ppara | GCACGCTTCCAGGAGATCAG | 6177 |
| Ppara | GAGGGTACATGCCTGACTCT | 6178 |
| Ppara | GGCAAGTAGGGAATGTTCTG | 6179 |
| Ppara | GAGGCGTTTCCTGAGACCCT | 6180 |
| Ppard | GTGCACCCGATGCACTTTCA | 6181 |
| Ppard | GTGAGACCTAGAAGAGCAAG | 6182 |
| Ppard | GGGCGAGTGCTTAGTTGTGG | 6183 |
| Ppard | GCGGCGATTGGCTACTGCTA | 6184 |
| Ppard | GCTGATGATGGCAGCTGATG | 6185 |
| Ppard | GCCAGAAGGCAATCTGTCAC | 6186 |
| Ppard | GCTTGGGAGAGGCATATGGT | 6187 |
| Ppard | GGCAGCCTCCACTCCTAGTG | 6188 |
| Ppard | GTCAAAGGAGTGGCTCCAGG | 6189 |
| Pparg | GTCCTCAAATAATAAGACAC | 6190 |
| Pparg | GCACTAAAGTCTGTTGATTA | 6191 |
| Pparg | GCTAGGTTGGCAAGGAATTG | 6192 |
| Pparg | GGACATCGGTCTGAGGGACA | 6193 |
| Pparg | GTAATACATTATTCTCAGGG | 6194 |
| Pparg | GTCTTCCCAACCTTCTTCCA | 6195 |
| Pparg | GTCTCTGTTATTATCTGGGT | 6196 |
| Pparg | GGTTCATATAGGGACTCTAA | 6197 |
| Pparg | GACCTGTGTCTTATTATTTG | 6198 |
| Ppargc1a | GTCTGAGCACCCAAGTGTTA | 6199 |
| Ppacgc1a | GCAGGGCTCCGGTTTAGAGT | 6200 |
| Ppargc1a | GTAGTTACTGTGTCAGTAAC | 6201 |
| Ppargc1a | GTTTCTCTTTGTCATCCATT | 6202 |
| Ppargc1a | GAAGCAAACAGCAAGCTTGT | 6203 |
| Ppargc1a | GGACTCCAACCCTAGTGCCT | 6204 |
| Ppargc1a | GTCGTTCCTGAGTCAATGAG | 6205 |
| Ppargc1a | GGCCCAGTGAGAAATGCACA | 6206 |
| Ppargc1a | GAGAGAGAGAGAGACAACCC | 6207 |
| Ppargc1a | GGAAACACTTGGCCTTTGGG | 6208 |
| Prdm1 | GCAAACAGAGGAAGCTGCCG | 6209 |
| Prdm1 | GGCGAAATAGGCTTGAGTCT | 6210 |
| Prdm1 | GCTAACAGCCTGTTTCTTCT | 6211 |
| Prdm1 | GTCCTGGAGCAAATACTTCA | 6212 |
| Prdm1 | GCTGTGGGTTTGGGCATGAG | 6213 |
| Prdm1 | GCCTATTTCGCCACCTCGGA | 6214 |
| Prdm1 | GCTGAATGTATTCAGTTAGC | 6215 |
| Prdm1 | GGGACAAGAGTAGAATACAC | 6216 |
| Preb | GATGGACAAACACTCTAACT | 6217 |
| Preb | GCGGAGGATGCTCTCAAAGT | 6218 |
| Preb | GGATGGACAAACACTCTAAC | 6219 |
| Preb | GTGCTGATAAACACCCACTT | 6220 |
| Preb | GTTGATTGGACTCTTTCTTA | 6221 |
| Preb | GTCTGTCCTCCACACAACCC | 6222 |
| Preb | GAGCATCCTCCGCGGTTGAT | 6223 |
| Preb | GTCCTTCCCTCTGCCCTTGT | 6224 |
| Preb | GGGAGAACCCATTTCCTCCC | 6225 |
| Prkaa1 | GCTCTGGATTCTCTCAAGGA | 6226 |
| Prkaa1 | GCAACATCCTGTTGACGTAA | 6227 |
| Prkaa1 | GCTATGTGAATTCCAGAAAG | 6228 |
| Prkaal | GCTTGCTTACACGTTGCCCG | 6229 |
| Prkaa1 | GTAAGCAAGCATCGTCCTCC | 6230 |
| Prkaa1 | GGCGTGCTTTGAAACAAGAC | 6231 |
| Prkaa1 | GCGTGCTTTGAAACAAGACA | 6232 |
| Prkaa1 | GGTGTCCCATAGTAATTTAC | 6233 |
| Prkaa1 | GGATGTTGCCAAGGAGCGAA | 6234 |
| Prkar1a | GACAATAAAGGAGACAGAAA | 6235 |
| Prkar1a | GAATACCCAGACTATGATAA | 6236 |
| Prkar1a | GTCTGGCATAATAGGAGGCT | 6237 |
| Prkar1a | GTGGCTGACACAGGAAATGG | 6238 |
| Prkar1a | GGTTTGACCCACACACGCTA | 6239 |
| Prkar1a | GGATGCTGAGATGCTCCTGA | 6240 |
| Prkar1a | GGTGCTTCTGTAGGCACGGT | 6241 |
| Prkar1a | GGCCAATGGATAGTTGAGCC | 6242 |
| Prkar1a | GGAGACTTCCAAGAGGAGCC | 6243 |
| Prkar1a | GTGGGTGCATGCTTCAAAGG | 6244 |
| Prkar1b | GCTGGGAAATGCTCATGTCA | 6245 |
| Prkar1b | GGTCAGTGTAGATAGACGAG | 6246 |
| Prkar1b | GGACGATTGGGCCAGAGACG | 6247 |
| Prkar1b | GCCCACATTCCCTATCGTTC | 6248 |
| Prkar1b | GTTGGTTCCATAATTCCTGA | 6249 |
| Prkar1b | GCACATGAGTTACTTAGGAA | 6250 |
| Prkar1b | GAGACATGTGTGTCAGGAGG | 6251 |
| Prkar1b | GAGTCAGGACAGGTGAGTGG | 6252 |
| Prkar1b | GAATGTGGGCAGGTGAGTGG | 6253 |
| Prkar1b | GTGGACTGGAGAATATAGAC | 6254 |
| Prkar1a | GCCTTTATGGGCGGTGCTAG | 6255 |
| Prkar1a | GCCTTTCAGTTAGCATAAAT | 6256 |
| Prkar2a | GCTGGGAACTGAGCTGTGTA | 6257 |
| Prkar2a | GCCCTCTGAATGCTGTCTGT | 6258 |
| Prkar2a | GTCCTGTAGCTGGACAGGCT | 6259 |
| Prkar2a | GAGGAAGACAACATGGGATG | 6260 |
| Prkar2a | GACGTCGTGAGATGTCAAAG | 6261 |
| Prkar2a | GGTCTACCTAGCTTACAGCC | 6262 |
| Prkar2a | GGTTTGTGCCAGGCCTTTAT | 6263 |
| Prrx1 | GGTGCTTTCGGAGATGCCAA | 6264 |
| Prrx1 | GATACTCCAGAAGACTGTCA | 6265 |
| Prrx1 | GTTCTTCCTAGAAGGTCTCC | 6266 |
| Prrx1 | GGCACTTAATGATATTTCTG | 6267 |
| Prrx1 | GGCTCTCTACGATCTAAAGA | 6268 |
| Prrx1 | GACTGATGCTGTCTGGCCTT | 6269 |
| Prrx1 | GAACATTTGATGCCATCAAA | 6270 |
| Prrx1 | GCTACAGGTTTCTAGAACAA | 6271 |
| Prrxl | GTGCTAATCTGTATCCAGTT | 6272 |
| Prrx2 | GCAGTGGCACCTGTAATCCC | 6273 |
| Prrx2 | GTCAGCAATGAATGGATGAT | 6274 |
| Prrx2 | GGGAACAATGAGGGACAATC | 6275 |
| Prrx2 | GGTCTTCAGGGTGTCAGAGG | 6276 |
| Prrx2 | GGATGGGTGGTTGGGTTCTG | 6277 |
| Prrx2 | GGACTTGCCTCCCAGAGGGA | 6278 |
| Prrx2 | GTTAAGCCTTGCTACTTACT | 6279 |
| Prrx2 | GTCTTTCTGGCAGTAGCAAG | 6280 |
| Prrx2 | GGGAAGTAGAGACAAGCCCT | 6281 |
| Prrx2 | GGACACCCATAACTGATACA | 6282 |
| Ptf1a | GCTGTCTGTTGAGGAACTGC | 6283 |
| Ptf1a | GCCCTCTCCAACCTCAAGAA | 6284 |
| Ptf1a | GTCTGTGAGAAAGTGTGTCT | 6285 |
| Ptf1a | GCCAGTCAGAAAGGTGAAAC | 6286 |
| Ptf1a | GGATTCTTGCAAGTTTGCGA | 6287 |
| Ptf1a | GTGGACTTTATCAGCTTACT | 6288 |
| Ptf1a | GCCCACTGCCCAGATAATTC | 6289 |
| Ptf1a | GCCACTTTCTAGTGAGATGG | 6290 |
| Ptf1a | GCCCAGATAATTCTGGATTC | 6291 |
| Ptf1a | GGTTATATATTCTTCTTGCA | 6292 |
| Ptn | GCCTGGTAATGTGTGTACCA | 6293 |
| Ptn | GTTAGTTTCTCCCAGCAGGA | 6294 |
| Ptn | GTAAGCCATAACAGTTTCCC | 6295 |
| Ptn | GCTGTCACAGCCATGTTCAT | 6296 |
| Ptn | GATGTGCATAGCCTGGGAGT | 6297 |
| Ptn | GCAATTTGTGTGTGGAAAGG | 6298 |
| Ptn | GTCATCTTCTTAGCTCACTG | 6299 |
| Ptn | GGTATCTGTCACTGAGGGAT | 6300 |
| Ptn | GTTTCTCCCAGCAGGAGGGC | 6301 |
| Ptn | GAAGAACAATCCCATGAACA | 6302 |
| Ptn | GGAGGGCAAGGGAGCTGAAG | 6303 |
| Ptx3 | GCTTCACTTATTTGAGATCA | 6304 |
| Ptx3 | GGGAAGGGTCACACTGAACG | 6305 |
| Ptx3 | GGAGCAGAGGAATTTACCTA | 6306 |
| Ptx3 | GCCACTCATGAGTCTCTGTC | 6307 |
| Ptx3 | GGTGAGGAGCACAGAGGTAC | 6308 |
| Ptx3 | GGTCTTGAGAAAGTATCCAA | 6309 |
| Ptx3 | GTGAGGAGCACAGAGGTACA | 6310 |
| Pura | GAAGCATAACAACCAAGAGT | 6311 |
| Pura | GATGGAAGTGATCATCAGGA | 6312 |
| Pura | GCCAGCAAACCATGCAGCCT | 6313 |
| Pura | GCCAGGAAGTTTCTTCAACA | 6314 |
| Pura | GGTCATCGAAAGATAGTTTG | 6315 |
| Pura | GTCAACAATAAAGTACAGTT | 6316 |
| Pura | GGCAGCGAGCCTTTCATCTC | 6317 |
| Purb | GAGGAACATATGTCTCCAGA | 5318 |
| Purb | GATTACCCAATACATAGTAC | 6319 |
| Purb | GAAACCGTATTTAGAATAGG | 6320 |
| Purb | GTGGAGGAGCCACATGGACA | 6321 |
| Purb | GGTCAGTCTAGTATTGGGCT | 6322 |
| Purb | GATGTTAACAGGTGGAGATA | 6323 |
| Purb | GGAGCAATCACATAAAGCAA | 6324 |
| Purb | GATTTGAGCAGTGTGTCCTG | 6325 |
| random | GACTGTCGTATTGCGAAACT | 6326 |
| random | GTTACGATTTGTGCCCGGCC | 6327 |
| random | GCACCGCCGTACTCTACACT | 6328 |
| random | GGGCGCACGATGTCGTATAG | 6329 |
| random | GGGTGCAAAGTAACGCGGCC | 6330 |
| random | GTCCGAGGAGTACGAAGGGT | 6331 |
| random | GTCAAGTGGACTGCGAGGGT | 6332 |
| random | GGTTACGGGCGGAGAGGGAT | 6333 |
| random | GCGGTATAACTACCAAGGGT | 6334 |
| random | GCGCCAGCGATGTTGAGGGT | 6335 |
| Rara | GAAACAGGGTGCCTGGCTGA | 6336 |
| Rara | GGAGAGAACTGAGGCACACT | 6337 |
| Rara | GACTTCTTGAAACCTGTTTG | 6338 |
| Rara | GGCTTGTCTGTAAAGGTTCT | 6339 |
| Rara | GGCGGAAATCCAGACGGGAG | 6340 |
| Rara | GCACACTCCACTCCACTCCA | 6341 |
| Rara | GGTTCTCACACCCTTCAGCC | 6342 |
| Rara | GGTGAAGGGCCAGAAGACCA | 6343 |
| Rara | GAAGGTAGGTAGCACAGCTC | 6344 |
| Rara | GCATAGAACCTGGCTGGGTA | 6345 |
| Rarb | GCTGGGCATTCAGAACACAT | 6346 |
| Rarb | GGCTCCTGATGGGCAGTTCG | 6347 |
| Rarb | GCTTCAATATGCCTTGCCCA | 6348 |
| Rarb | GGGACTTCACAAGGAAGCTG | 6349 |
| Rarb | GCTGAAGGGAGAGCTCTCAC | 6350 |
| Rarb | GATTGGCGTGGGTTCAGACA | 6351 |
| Rarb | GAAGGTTAGCAGCCCGGGAA | 6352 |
| Rarb | GCTGGGAATTCTCCCACAGG | 6353 |
| Rarb | GCAGCAGCCTCCTGGAGAAA | 6354 |
| Rarb | GGGAAGGACACTGACACACA | 6355 |
| Rarg | GGCAAAGAAGGCGGGAACGG | 6356 |
| Rarg | GACCTTTCAGATTTCGGAGG | 6357 |
| Rarg | GTAGACTCCGCTGCGCTGGA | 6358 |
| Rarg | GAGTTTGGCCTGGACTGGGT | 6359 |
| Rarg | GATGGTCACGACTCCAGAAG | 6360 |
| Rarg | GAAGAAAGGGTTGAAGGGAG | 6361 |
| Rarg | GCATCTGCTGGAGAGAAAGG | 6362 |
| Rarg | GCGTAGCGCAAGGCATCTCA | 6353 |
| Rarg | GGGTGTGAGGGAGAAAGCCC | 6364 |
| Rarg | GTGATATCCTGGAGGTCAGA | 6365 |
| Rax | GAGCTTGTGGTTATTACTGC | 6366 |
| Rax | GTCCTGTGGGACTGGAACCT | 6367 |
| Rax | GACTAACACTTCAGTGAGGC | 6368 |
| Rax | GTGGCTGAACCAGTGCATGC | 6369 |
| Rax | GTCAGCCACAGGTTGGAGCT | 6370 |
| Rax | GTGGGAGGTTTGACAGGAGG | 6371 |
| Rax | GATTAAGGGAAGATTCAAAC | 6372 |
| Rax | GAATCCTGCATCTCTGAGGC | 6373 |
| Rax | GAGCCAGTCAGGACCATTGT | 6374 |
| Rb1 | GGCTACATACAGTCTAGGTT | 6375 |
| Rb1 | GAGGAATCGAGAACTTAATT | 6376 |
| Rb1 | GGAATGTAGCCCAGGAGAGG | 6377 |
| Rb1 | GGCTGTCCTGTGTTCTCATG | 6378 |
| Rb1 | GGTGAAGGAGAAATGGAGGC | 6379 |
| Rb1 | GACAGCCGGTCAAACTGGGA | 6380 |
| Rb1 | GCTCAGGCTACATACAGTCT | 6381 |
| Rb1 | GTTCACTTTGTCCCAGATTT | 6382 |
| Rb1 | GTTTCTGGTAGTTTGCCACC | 6383 |
| Rb1 | GGAGGTGTCCAATAGCCAGG | 6384 |
| Rbl1 | GCTTTGACCCTCGATGGAGG | 6385 |
| Rbl1 | GTCTTACATGACGTATCGGA | 6386 |
| Rbl1 | GGATGCAGGGACAAGGGTTT | 6387 |
| Rbl1 | GGTCCTCGCGCTTACCTGAA | 6388 |
| Rbl1 | GATGCAGGGACAAGGGTTTG | 6389 |
| Rbl1 | GGGTCCACAGGACAGTGTCA | 6390 |
| Rbl1 | GGGTCAAAGCTCAGTGAGAG | 6391 |
| Rbl1 | GGGATGCAGGGACAAGGGTT | 6392 |
| Rbl1 | GAACTAGCTTGATATTCCCA | 6393 |
| Rbl2 | GCAACACATGTGAGTTATAC | 6394 |
| Rbl2 | GTCAACGCTATCAATGGCAA | 6395 |
| Rbl2 | GAGTTATACAGGTTGATCTC | 6396 |
| Rbl2 | GTATAACTCACATGTGTTGC | 6397 |
| Rbl2 | GCCATTGATAGCGTTGACAT | 6398 |
| Rbl2 | GACATCAAGAGCAGGGCTGT | 6399 |
| Rbl2 | GCTAAGGCAGCCCAGACACC | 6400 |
| Rbl2 | GATTACATGCCATTAAGCTC | 6401 |
| Rbmxl1 | GGAGTTCCTGCTGACGTGTG | 6402 |
| Rbmxl1 | GACTTGCTCTGCTGCATGTC | 6403 |
| Rbmxl1 | GACCACATCAGAGGTTAGTT | 6404 |
| Rbmxl1 | GCTTGGCCGTGTATGCACGG | 6405 |
| Rbmxl1 | GTAATGTGACCTGTGGACAC | 6406 |
| Rbmxl1 | GTCGGGATAGTGGTGGCGAT | 6407 |
| Rbmxl1 | GTTAGGGAACAATGGAAACC | 6408 |
| Rbmxl1 | GAGATTATTGGCGGGAGCGG | 6409 |
| Rbmxl1 | GAGTCACGGGCTGCACTAAC | 6410 |
| Rbp3 | GTTTACCAAGTGGTTTAGGA | 6411 |
| Rbp3 | GGTGTAGAAAGCATATCACA | 6412 |
| Rbp3 | GCCTGGAGTTGGATTCTTCC | 6413 |
| Rbp3 | GGCTCCATTGCTCTTAGGCC | 6414 |
| Rbp3 | GTCTGACAGGCACTATGACA | 6415 |
| Rbp3 | GAAACCTACAAGTCAGTTTC | 6416 |
| Rbp3 | GGCACACAGAGGCCTCTGTG | 6417 |
| Rbp3 | GGGAGAGGGTGAAGACTACC | 6418 |
| Rbpj | GTTGTGTCTGAGCCGAGCGG | 6419 |
| Rbpj | GATGAAACAGTACCTAGAAC | 6420 |
| Rbpj | GGAAAGTACCAGGCTCGGGA | 6421 |
| Rbpj | GGCTTGTAGTGGTGGCTCTG | 6422 |
| Rbpj | GCAGCCAGCTTGCAGGATGT | 6423 |
| Rbpj | GCAGGGAGGATCAGCAGGTA | 6424 |
| Rbpj | GTCGTGCGGGAGCAAATGAA | 6425 |
| Rbpj | GGAAAGCAAAGGGCCGGGAA | 6426 |
| Rbpj | GAAAGCAAAGGGCCGGGAAG | 6427 |
| Rbpj | GAGCAGATCCCTTAGTCCGC | 6428 |
| Rbpjl | GCCCACTAGGAACTGCCTCA | 6429 |
| Rbpjl | GGTCTGGCTTCTACTGCCGT | 6430 |
| Rbpjl | GGAAGGATGGGTGTGTGTAT | 6431 |
| Rbpjl | GCTCCAGTCTGCAGGTGAGT | 6432 |
| Rbpjl | GGGCAGATGTAGGCTTCCCA | 6433 |
| Rbpjl | GTCTTGGTCATCGTGACAGA | 6434 |
| Rbpjl | GAGTTTGGCCTGCAGTTCCA | 6435 |
| Rbpjl | GCTCCCTTCCTCCCATGGTG | 6436 |
| Rbpjl | GAGAGAGTGGCCAGCAGCAG | 6437 |
| Rbpjl | GCATCTGCATTGCCTGAGCT | 6438 |
| Rel | GTGACGTCATGCTGGCCGAG | 6439 |
| Rel | GATATTCCACATCATAGACC | 6440 |
| Rel | GAGAGCGCCGCTTAAAGCCG | 6441 |
| Rel | GAGATTGGCCGAGATACCCA | 6442 |
| Rel | GCATTAGTATGCAGTATGCA | 6443 |
| Rel | GCCTCAGGCCGTGGGTATCT | 6444 |
| Rel | GCTCTCAAGGACTCTGGAGG | 6445 |
| Rel | GCATACTAATGCTGAGAGAA | 6446 |
| Rela | GCTGCCTCCACTATGCCAGA | 6447 |
| Rela | GCTGAAACCTCCTGTGGCCC | 6448 |
| Rela | GCTGCATCCGACAGGCCTTA | 6449 |
| Rela | GGCCTTGAAGGAGATGTTCA | 6450 |
| Rela | GAAACTGAAATGAGTGGGAG | 6451 |
| Rela | GTTCATGTGAGGACCAATGA | 6452 |
| Rela | GATGGGAAGAGCCTGAACTC | 6453 |
| Rela | GCGCAGCCGGATCTAGGTTG | 6454 |
| Rela | GAGAAACTGAAATGAGTGGG | 6455 |
| Relb | GACGTCACGTCAAAGGGAAT | 6456 |
| Relb | GTACCAGTGGGCAGAGCCTC | 6457 |
| Relb | GTTGGTTTGCGTTGAGACAA | 6458 |
| Relb | GGCTGCTAACTTCCCACTCC | 6459 |
| Relb | GGTGTGTGTGTGTGTGTGAC | 6460 |
| Relb | GACAGTACATGGTGCATCCA | 6461 |
| Relb | GACATATCCAGTGCCCTTAT | 6462 |
| Relb | GACAAGGGCCTGCTATGCAG | 6463 |
| Rest | GATCTCAGCGCCGTGCGGAA | 6464 |
| Rest | GGAGCCGCACATTCCAGCAC | 6465 |
| Rest | GCATAAAGATCTGTGTAGGC | 6466 |
| Rest | GCGTCCTGTGCTGGAATGTG | 6467 |
| Rest | GAGGTTCACACATTTAGATC | 6468 |
| Rest | GAAATAGTAACAAAGTAGCC | 6469 |
| Rest | GGAAACTTACCTAATCCGCC | 6470 |
| Rest | GACAATACTTCTCAAGAGGG | 6471 |
| Rest | GTACCTGACGTCTTATAATG | 6472 |
| Rest | GAGGCTGGAAGCAGAGCTTC | 6473 |
| Rfx1 | GACTGCATTTGTTTGACATT | 6474 |
| Rfx1 | GAAGTACAACCAAGTCTTCT | 6475 |
| Rfx1 | GCAGCCCTGAGAATTACAAG | 6476 |
| Rfx1 | GCTGCAACTGACCTATCTCT | 6477 |
| Rfx1 | GCTCTCTTGCACAGGTCCAC | 6478 |
| Rfx1 | GGCGTGTGACGTAGTAGGGT | 6479 |
| Rfx1 | GGCCACAGATAGAGCCTGTA | 6480 |
| Rfx1 | GTGACAGGTGAACACATACA | 6481 |
| Rfxl | GTCTTCACAGAAGTGGCAGT | 6482 |
| Rfx1 | GCCAGGCTGTGACCTTTCCG | 6483 |
| Rfx2 | GGAGGCGCTCACCGTCTAAG | 6484 |
| Rfx2 | GTGCCACAACACCACTTTGT | 6485 |
| Rfx2 | GGTCTTTAGCAAGGAGACTG | 6486 |
| Rfx2 | GAGGACAGAGAAGCGAGATG | 6487 |
| Rfx2 | GTAACTATCATGGAGGCAAA | 6488 |
| Rfx2 | GGTTTCCAACCCATGAGCTC | 6489 |
| Rfx2 | GGGAAGGGTTGGCTCTAAGG | 6490 |
| Rfx2 | GCCTTGCGCCTGCTCTTTCA | 6491 |
| Rfx2 | GGGCACTATGAAAGCCAATG | 6492 |
| Rfx2 | GAGGTCATGGGTTGGAAACC | 6493 |
| Rfx3 | GTCGCCAGTGTGGTGTTTCC | 6494 |
| Rfx3 | GAGAAAGCGGAATTCGATGG | 6495 |
| Rfx3 | GCGCGCGTCTCACACAGTGT | 6496 |
| Rfx3 | GAACGGTGACCCATCTCGCT | 6497 |
| Rfx3 | GTTAAGTGATGGGGAGGTAA | 6498 |
| Rfx3 | GTGTGTGTGTGAGAGAGGGC | 6499 |
| Rfx3 | GCACTCACAAGGTTGACATA | 6500 |
| Rfx3 | GGTAACTTCTTACTCTGGTA | 6501 |
| Rfx3 | GCGCTTGATTCACAAGGCAA | 6502 |
| Rfx3 | GTGACTGACAGCTCGGAGCC | 6503 |
| Rfx5 | GACCACGCAGAACGAGGCAC | 6504 |
| Rfx5 | GCGTGTATCCAGGCAGATCG | 6505 |
| Rfx5 | GAGATCTCTTTGGGTATACA | 6506 |
| Rfx5 | GGAAAGGGTTCTGTTCTTAA | 6507 |
| Rfx5 | GGATGTCGTGGGATGACGTA | 6508 |
| Rfx5 | GGGAACAGGGTCCCAGATTC | 6509 |
| Rfx5 | GGCTGGAGGTGTTGCAGCAC | 6510 |
| Rfx5 | GGCTGCCTCAGGTCTTGGTT | 6511 |
| Rfx5 | GATCGACCGCGGGCTTTACT | 6512 |
| Rfx5 | GAAACATGAATAGGCAAAGG | 6513 |
| Rfx7 | GAGAATTGACAAAGTGGCTG | 6514 |
| Rfx7 | GAGTGCGTTTACGGCGACTT | 6515 |
| Rfx7 | GCTTCCAGTCTGTATGAACC | 6516 |
| Rfx7 | GCCTCACATTGCCACATTCG | 6517 |
| Rfx7 | GTCTGTATGAACCAGGCCTG | 6518 |
| Rfx7 | GTTACCTTAAACCTTGGGTA | 6519 |
| Rfx7 | GAGCTGTGTGTCTCCGAGCC | 6520 |
| Rfx7 | GCTGTCTATGAATGGGAAGG | 6521 |
| Rfxank | GCAGTCCTATGCGAGCGTGT | 6522 |
| Rfxank | GGGATGGTCTCTAGACAGCA | 6523 |
| Rfxank | GTAGCAGGGAGTCCTTGACG | 6524 |
| Rfxank | GGAGTTAGAGAGGAGCCGCC | 6525 |
| Rfxank | GCTGTGTGGGAACAGCTCTG | 6526 |
| Rfxank | GGTGTTGCGGGACCCTAGAG | 6527 |
| Rfxank | GTCATCTGCAGGTCCAGGGA | 6528 |
| Rfxank | GTCATCTGCCACCATTAGCC | 6529 |
| Rfxank | GTATGCTCCAGTTATAAAGG | 6530 |
| Rfxank | GAGTTAGAGAGGAGCCGCCA | 6531 |
| Rfxap | GACAGCTGCGCATGCGCAAT | 6532 |
| Rfxap | GGCACGTTCTTTAGCTTCCT | 6533 |
| Rfxap | GCCATCCGCAAGCGATTCCT | 6534 |
| Rfxap | GTGCAGGCTGACCAAGAACC | 6535 |
| Rfxap | GAACAGTGCCTAGTGAAGTT | 6536 |
| Rhox11 | GGAGGCACTTCCCTTGTCTG | 6537 |
| Rhox11 | GACAATGATCACTCTGGAGA | 6538 |
| Rhox11 | GCTAATGCTACTGACTGTCT | 6539 |
| Rhox11 | GATTTCCCAAACAGGCTTAC | 6540 |
| Rhox11 | GAATAAGTCACCCATATTGA | 6541 |
| Rhox11 | GTACCTGAAAGTTCTGTATT | 6542 |
| Rhox5 | GAGGTCTTCAGGAAGCTGTT | 6543 |
| Rhox5 | GAACATTGGGATGATGTCAT | 6544 |
| Rhox5 | GTAATTGCCTCCCATTCACT | 6545 |
| Rhox5 | GGCTAAAGAGGAGAGAACAT | 6546 |
| Rhox5 | GGTCTCTCTTCCTTTGTCTA | 6547 |
| Rhox5 | GGATGTGGCAATGTATCTCA | 6548 |
| Rhox5 | GTCTCTCTTCCTTTGTCTAT | 6549 |
| Rhox5 | GAAGAAGGAGGAGGAGGAGG | 6550 |
| Rhox5 | GAAAGTGTGCACTTTCTCAA | 6551 |
| Rhox6 | GCCCTAACTACACAGGCTAT | 5552 |
| Rhox6 | GCTGACACTAGCTGCAAGCC | 5553 |
| Rhox6 | GGCTTGCAGCTAGTGTCAGC | 6554 |
| Rhox6 | GAAATGTATCTCAGAATCCA | 6555 |
| Rhox6 | GCACGAGGAGTTATGCTTAG | 6556 |
| Rhox6 | GATTAGCTGCTCTACAAGCC | 6557 |
| Rhox6 | GCTTTCCTTTAGGACTTGGT | 6558 |
| Rhox6 | GCAGAGGTGCTAGGGCTACA | 6559 |
| Rhxo9 | GGAGACTCTGATGGGCGAGT | 6560 |
| Rhox9 | GGTTGTGCAAGCTCAGTATG | 6561 |
| Rhox9 | GCTTCTTCCCTGAGGCGCAC | 6562 |
| Rhox9 | GAGATGGCAATGAACAGACT | 6563 |
| Rhox9 | GGCCTGAGCAGTGACTGTGA | 6564 |
| Rhox9 | GCTAGAAATTTCGGAGCCCG | 6565 |
| Rohx9 | GAGATTCAACTGGATCCTGG | 6566 |
| Rnf2 | GAACATTCCGCTTTGATGGA | 6567 |
| Rnf2 | GGTCGATATAAAGAGTAGCA | 6568 |
| Rnf2 | GTTCCTCCATCCTCTGGAGA | 6569 |
| Rnf2 | GAGTAGCAAGGAGATCATTA | 6570 |
| Rnf2 | GAGGGAAGGCGCAAACCTGT | 6571 |
| Rnf2 | GAACACTGAAAGCGTCAAGG | 6572 |
| Rnf2 | GCGCCTATTTCCAGAGTTGA | 6573 |
| Rnf2 | GTTTCTGCTGCAGAACTTTC | 6574 |
| Rnf2 | GACTACCCGTCTCCAGAGGA | 6575 |
| Rnf2 | GGAGCTCGGGAAATACAACA | 6576 |
| Rnf6 | GACTCTGAGCTTCGCGCCTC | 6577 |
| Rnf6 | GGACACTGAAATACGAGAAG | 6578 |
| Rnf6 | GTGTTTAACATGTCCCTGGA | 6579 |
| Rnf6 | GTTGCTGCTCGGAGTCGACC | 6580 |
| Rnf6 | GAGCTATGTGATGGAGACAG | 6581 |
| Rnf6 | GGCATTTCCAAAGGGRGGAA | 6582 |
| Rnf6 | GGAGAGGAGGACGTGCTAGG | 6583 |
| Rnf6 | GTTTCTGCCTGTGCCCGGTT | 6584 |
| Rnf6 | GTTCCGCATCTGCTGTGCGC | 6585 |
| Rnps1 | GATTGTGAGAACTGAATCTT | 6586 |
| Rnps1 | GGATCTAAGGCACCCTGCTG | 6587 |
| Rnps1 | GTGTTGGCAAAGTCGGGAGG | 6588 |
| Rnps1 | GGACAGCAACTGTGTGTGGG | 6589 |
| Rnps1 | GTCAGAGGTGAGAAGCGGGA | 6590 |
| Rnps1 | GCGCGCGATGATTGGCTGAC | 6591 |
| Rnpsl | GCCTACCGGATCTGTGTGGA | 6592 |
| Rnps1 | GACTTCAGCCGTTCTTCGTA | 6593 |
| Rnps1 | GGAGCATAGAGTACTTACCA | 6594 |
| Rora | GAGACTGTAGCTTCCTCAGA | 6595 |
| Rora | GTTGGCTAATCTCAGCCAAG | 6596 |
| Rora | GAGATAAAGTCTGCTCCCTT | 6597 |
| Rora | GACGTTATTAATACCTCTCC | 6598 |
| Rora | GAATTTCAAGACTACTTACC | 6599 |
| Rora | GAAAGATCCCAGAGAAGGGT | 6600 |
| Rora | GCCTTGTGTAGCTCCCGGTC | 6601 |
| Rora | GAGTCAGAAGTCTGGCGGGC | 6602 |
| Rora | GAGGTGGAGAAGAGGGAGGC | 6603 |
| Rora | GAAGCTGACTGACAACCCTC | 6604 |
| Rorc | GTCAGAGATGACCTAGTCAG | 6605 |
| Rorc | GAATATTGGATGCCTCAGTT | 6606 |
| Rorc | GCGACTCTCAAGCCAGGACC | 6607 |
| Rorc | GAACAAATAGTTGAAGCTGT | 6608 |
| Rorc | GAATGGAATGCTGGGAGCGA | 6609 |
| Rorc | GAAGAGCAAATTGAGAGGTG | 6610 |
| Rorc | GTTGGGTAAGCAGGAAAGCC | 6611 |
| Rorc | GGCACAGCTAATCAAACTCT | 6612 |
| Rorc | GATATACTTCCCTGCAGCTT | 6613 |
| Rorc | GCAGAAGAGAGCAACTGCAC | 6614 |
| Runx1 | GCAGCTGGGACTCTACCGAG | 6615 |
| Runx1 | GGGTCGATCTTGTGAGTTTG | 6616 |
| Runx1 | GAAGTCCAAGCAAGACTGCA | 6617 |
| Runxl | GCACAGAGACTTGAATAATG | 6618 |
| Runx1 | GAGCACAGATGAAAGTGGAG | 6619 |
| Runx1 | GTTTGCATAGAGGAGACCGA | 6620 |
| Runx1 | GAATCCCACCCTGTCCTCCC | 6621 |
| Runx1 | GGATCTCTCCAAGGCAGAGC | 6622 |
| Runx1 | GCAGCTACAGGCTTGGATCC | 6623 |
| Runx1 | GTGAGCCCTGCAGTCTTGCT | 6624 |
| Runx2 | GATAGTGTCGATAGTGGGAG | 6625 |
| Runx2 | GTAAGTGGTGCAAGCAGAAA | 6626 |
| Runx2 | GTACAAGGAATCGCAGCACT | 6627 |
| Runx2 | GAGGGAGACTGAGTGGCTCA | 6628 |
| Runx2 | GCGCTAGGGAGGGTCATGAC | 6629 |
| Runx2 | GCGAGGATACAAGTTAGTTT | 6630 |
| Runx2 | GCTCAGAATTTGAGGCTGGT | 6631 |
| Runx2 | GGCGGATTTCCCGGCTTCTG | 6632 |
| Runx2 | GGAAGTCGGGTGGGAGATGT | 6633 |
| Runx2 | GCGGATTTCCCGGCTTCTGT | 6634 |
| Runx3 | GCAGCTCAGGACCAGAGGTT | 6635 |
| Runx3 | GATGTCTGCCCAGGTCGCAG | 6636 |
| Runx3 | GAAACAGCCACTGCTGGGAG | 6637 |
| Runx3 | GAAACAGCCTCTGGACCAGA | 6638 |
| Runx3 | GCTATAACCCTCGGAAGACG | 6639 |
| Runx3 | GTTGACCATCACTAGGCCTT | 6640 |
| Runx3 | GGTGTCAGGGTAGTGGTAGA | 6641 |
| Runx3 | GGCCTGGCCTTGTGGTTCTG | 6642 |
| Runx3 | GGCGGCGCCTTTCTGTTGAA | 6643 |
| Runx3 | GGTGAGAAGTTAGAAAGTGG | 6644 |
| Rxra | GCTGAAGACTGTAGTCAGGC | 6645 |
| Rxra | GGTTTGAACTCAGTGCGAGA | 6646 |
| Rxra | GAGCTAAAGCACCATCAACA | 6647 |
| Rsra | GAGGAGATCAAGGTCCTATG | 6648 |
| Rxra | GGGTACCCACGTTAACACGA | 6649 |
| Rxra | GATTAGGGTTCAGGGATTCC | 6650 |
| Rxra | GGTGTGTCATCCTGACTCAA | 6651 |
| Rxra | GCCACTATGACCCTAGAAAT | 6652 |
| Rxra | GAGTTGTTAGGGCTGACTGC | 6653 |
| Rxra | GACTGCAGAAGCCTTGGATC | 6654 |
| Rxrb | GGGACTGGTGTGCTGGGAAA | 6655 |
| Rxrb | GATTGTCGCCTTCCTCGTGG | 6656 |
| Rxrb | GCCATGTTGGTAAAGGTATC | 6657 |
| Rxrb | GGGACTGGTTCTTAATCGGT | 6658 |
| Rxrb | GCAGTGGACAGTGACGTGGC | 6659 |
| Rxrb | GACTCTCAATCTACCTATTC | 6660 |
| Rxrb | GCCGCCATCTTTGTACAGAC | 6661 |
| Rxrb | GACGGTGGGAGTCTAGGAAA | 6662 |
| Rxrb | GGCCGCCATCTTTGTACAGA | 6663 |
| Rxrg | GACACAGGGACTAGCAGGCT | 6664 |
| Rxrg | GAGTTCTCTGATATGGCCTT | 6665 |
| Rxrg | GGCATAGTGCAGCTCGCCAG | 6666 |
| Rxrg | GTAACAAGGGCCAATGTCAC | 6667 |
| Rxrg | GTTGCTAGTTTGATTAACTC | 6668 |
| Rxrg | GGACTAGAGAGGCCATTCCA | 6669 |
| Rxrg | GAGTTGGTTGGCTCTAACCA | 6670 |
| Rxrg | GGCATACGGCCCAGGAATAG | 6671 |
| Rxrg | GCTCCTTTAGCCTAAGACAC | 6672 |
| Rxrg | GTTATTTGATCTGTGAAAGG | 6673 |
| Sall1 | GGGTGCTCAAACTGCACAGA | 6674 |
| Sall1 | GGGACACAGCCAGAGCGCTT | 6675 |
| Sall1 | GGAAACCCTGTCTTGCCGCG | 6676 |
| Sall1 | GTTCCAAGGCTCTGCTGTGA | 6677 |
| Sall1 | GAATTGGTCTTTATTGTTGG | 6678 |
| Sall1 | GGTGGCGATACATCAATTAC | 6679 |
| Sall1 | GCTGATTGCTGGAGAAGTGA | 6680 |
| Sall1 | GACATGGGTCCTGAGTTCCA | 6681 |
| Sebox | GGAACTGGCATGGTGTGCCA | 6682 |
| Sebox | GCCTCTGGAGGGAAGAGGCT | 6683 |
| Sebox | GCCTAACACAGCAGAAGGAG | 6684 |
| Sebox | GGGACTGAGTTGTGTGTCTT | 6685 |
| Sebox | GTACTCAGGGTGGAGGAGAA | 6686 |
| Sebox | GTTAGGCAAAGTCCAAGGTA | 6687 |
| Sebox | GTCTATTTCTTCTCTGGAGG | 6688 |
| Sebox | GGCCTTCCTAGTTAACTTCA | 6689 |
| Sebox | GCACTTTGCCTTGCTTCAGC | 6690 |
| Sebox | GGAAGAATTTCACAAAGTAC | 6691 |
| Setdb1 | GGGAAACAGCGTGAGGAGGC | 6692 |
| Setdb1 | GAAGACAGTGTACTTGAGTT | 6693 |
| Setdb1 | GCAAACTGAAGGAGAGACGG | 6694 |
| Setdb1 | GCAGCGCTATGCAATAAATT | 6695 |
| Setdb1 | GCCAAACCCAGGCAAACTGA | 6696 |
| Setdb1 | GTCTCTCCTTCAGTTTGCCT | 6697 |
| Setdb1 | GAGACTGTGGTACACCTCTG | 6698 |
| Setdb1 | GTAGGGCATTTCCAGATAAG | 6699 |
| Setdb1 | GGAGTTTACTTACACAGCAG | 6700 |
| Shh | GGTTCAGCTATTCCTCCTGC | 6701 |
| Shh | GACCAAGTACAGATTCTTAG | 6702 |
| Shh | GGCGAACTATTTATGTGGAA | 6703 |
| Shh | GCATTTCTGCAACCTGGAAC | 6704 |
| Shh | GGAAGCAGGACTAGGCTCTT | 6705 |
| Shh | GAAATTCTGCAGTCTCCAGT | 6706 |
| Shh | GGGATGTACACAGAGGATAC | 6707 |
| Shh | GCAAGCTGTCCCTGGGTACG | 6708 |
| Shh | GCCTCTGGGAGTTAAATGGC | 6709 |
| Shh | GGTAAAGGIGGGTGGGAGGG | 6710 |
| Shox2 | GCAGGGTGCAGGGAGTTGTT | 6711 |
| Shox2 | GCATTGCAATCAGAGTCAGT | 6712 |
| Shox2 | GCAGCGGCTTGGAGCAAGAA | 6713 |
| Shox2 | GGGTCTCCTGATCTCTTACC | 6714 |
| Shox2 | GAACTGGATAGACTTCTCGG | 6715 |
| Shox2 | GATGGCGAGGAAGGGAATGG | 6716 |
| Shox2 | GGGAAATGTTTCTAGAAGGA | 6717 |
| Shox2 | GATGCTAAATAATTAAGGGC | 6718 |
| Shox2 | GATCCAGGCTGGAGTCCACC | 6719 |
| Shox2 | GGGATGGCGAGGAAGGGAAT | 6720 |
| Sin3a | GAGGTTCCAGCCACTAGCCT | 6721 |
| Sin3a | GCCAAGCCAAGCCCTGTTCC | 6722 |
| Sin3a | GAAGGATGCTAAAGGCTGGA | 6723 |
| Sin3a | GTAAATCTCTTCCTAGTTCA | 6724 |
| Sin3a | GAGGCAGCTCCATGTTTGCG | 6725 |
| Sin3a | GTTACTAGATGAAAGAGGGT | 6726 |
| Sin3a | GCTCCGCGCCCTTAGTTAGG | 6727 |
| Sin3a | GTAGATGAGGTTTACATTTG | 6728 |
| Sin3a | GTGATTGGCTAAACCATTGA | 6729 |
| Sin3a | GACTGAACCTCAGCCTTCCA | 6730 |
| Sin3b | GTGCAAGAATTCAGTCCACA | 6731 |
| Sin3b | GTGGTCAAGGTAGACACCTA | 6732 |
| Sin3b | GGAGACTCGTGGCGTCAAAT | 6733 |
| Sin3b | GTCACTCTGAGAGGAGTTAA | 6734 |
| Sin3b | GCTTTCTGGGACAAGGACTT | 6735 |
| Sin3b | GGAAGGAGAGTATACCAGGT | 6736 |
| Sin3b | GCTTGCTCTAAGCAAGCAGG | 6737 |
| Sin3b | GACAGAATCCTAGAGCAAGG | 6738 |
| Sin3b | GGTTTGCACACATTTGTGAA | 6739 |
| Six1 | GAAGCTACCGAGTGCTGCCT | 6740 |
| Six1 | GGAGAGGTGGGAAGTGAGGT | 6741 |
| Six1 | GCAAGTAGGTCCCAGATACA | 6742 |
| Six1 | GTGCTGCCTAGGATAAGAAG | 6743 |
| Six1 | GTGACACGTGGGAAGAGGAG | 6744 |
| Six1 | GGCTACTTACAATCTCTCCA | 6745 |
| Six1 | GTATAACTCACAGATAAGGA | 6746 |
| Six1 | GTGGAGATAAGGGAAGGTGG | 6747 |
| Six1 | GTCTCTATGCTACAGTGCCA | 6748 |
| Six2 | GGCAGTCTGCGGGTCTATGC | 6749 |
| Six2 | GTCTGTGCCTCCTTGGATCT | 6750 |
| Six2 | GAGGCCCTAGGCAGGATTGG | 6751 |
| Six2 | GTCCTGCCAATGCTGACAGT | 6752 |
| Six2 | GGGTAATTGTCGCACTTCCC | 6753 |
| Six2 | GTGGACTTCCTCTGTGGGTA | 6754 |
| Six2 | GGTGCAAATTCTGGGAAGGA | 6755 |
| Six2 | GAGCAGCTGCTTAAGAGGCC | 6756 |
| Six2 | GGCCTTAGAAAGTGGGTAGG | 6757 |
| Six2 | GAGCATAGGCTGTCTGGGTA | 6758 |
| Six3 | GTTTCAATACGCGTTGTACA | 5759 |
| Six3 | GGGTTTAAGGAGGAGCCGAG | 6760 |
| Six3 | GGCCAGAAACCTAGGGACTC | 6761 |
| Six3 | GATGACTTGCGACTAACTTC | 6762 |
| Six3 | GGGCCTAAACTCGCTGACCA | 6763 |
| Six3 | GGCTAAGATTAACAAGCAGG | 6764 |
| Six3 | GAGCACAATTTCCCAGGCAA | 6765 |
| Six3 | GGGAGGCAGCATAGGGCTTC | 6766 |
| Six3 | GTCACCGTAGCAAGCTGCTG | 6767 |
| Six3 | GAGATTCTCAATCTCCAATG | 6768 |
| Six4 | GGTGTGGTGGGAAGAGCAAG | 6769 |
| Six4 | GCAACCGGAGGAGTCACGTT | 6770 |
| Six4 | GGTCTTAGCTCAGAGAGGGA | 6771 |
| Six4 | GTTCCTAGTAGATTCAAACA | 6772 |
| Six4 | GGGTTGAGGCTGAAGGGAGG | 6773 |
| Six4 | GTAGCCCACCGAGATGACAA | 6774 |
| Six4 | GAAAGGCCCAGTGATTCCCA | 6775 |
| Six4 | GATCTTGAAGAGTGGAAGAG | 6776 |
| Six4 | GAGCTAAATTATAATGGACT | 6777 |
| Six5 | GCCCATCTATGGGTATAGGC | 6778 |
| Six5 | GACCCAGGCTCACAGAAGTG | 6779 |
| Six5 | GGTCCGAAACCGAGACCTGG | 6780 |
| Six5 | GTTTAGGGTCCATTCTCCTT | 6781 |
| Six5 | GGGTAGGCGGTGGATCTAGC | 6782 |
| Six5 | GGTGAGAACCTCTTCTTCCA | 6783 |
| Six5 | GGCGCATGTTCGGCAGCTAC | 6784 |
| Six5 | GGATCTGCAGGAGAGAAGGT | 6785 |
| Six5 | GTGGGTAGCATATCTAAGAT | 6786 |
| Six5 | GACAGACCCGAGTGCAGAGC | 6787 |
| Six6 | GTTCATCCCTGAATTGGACT | 6788 |
| Six6 | GGGAGGTGCTGAAACGACCG | 6789 |
| Six6 | GAAATGATAGGAATGGTTGC | 6790 |
| Six6 | GTTGGTAGAAAGGAAACACT | 6791 |
| Six6 | GACTTGCTTACAAAGGTTAA | 6792 |
| Six6 | GGCAGAGCTTGGCAGAGTGA | 6793 |
| Six6 | GCAGCATCCTACCTCTCTGG | 6794 |
| Six6 | GATTCTCCCTCTCCCTCAAG | 6795 |
| Six6 | GGGCTTATTAGTTGGTAGAA | 6796 |
| Six6 | GAGTGAGGGCCCTAGAGGAA | 6797 |
| Smad1 | GTGTATGGCCATACCCTCCC | 6798 |
| Smad1 | GAGGTGTCCAGGATGGCACT | 6799 |
| Smad1 | GAGGATCCCTAAGCGGCAGC | 6800 |
| Smad1 | GCCTGGCTTAAAGCCACTCA | 6801 |
| Smad1 | GTCTCTGGGAAGGGCTGTCC | 6802 |
| Smad1 | GTTTCTTGTTTAAGICCTGA | 6803 |
| Smad1 | GCCCTGAGTGGCTTTAAGCC | 6804 |
| Smad1 | GAAGGCGCGGGCCGGTAATT | 6805 |
| Smad1 | GCTCATAGTAGACAAAGCCA | 6806 |
| Smadl | GCTCATGCTACATGAAGGGC | 6807 |
| Smad2 | GGTGGGTAATAGATGATTCT | 6808 |
| Smad2 | GTGCGGTTGGTATTAGGGCT | 6809 |
| Smad2 | GTCTCCAGGAACATTGAAAT | 6810 |
| Smad2 | GTTTCTCCAGCCCGAGCCGT | 6811 |
| Smad2 | GAGCTCAAAGTCTGACACTT | 6812 |
| Smad2 | GGAAGTAGGCTGGAAACAGT | 6813 |
| Smad2 | GATGAAGAAGCTTGGAGGGT | 6814 |
| Smad2 | GGTGACCCGGTACCTTTAGT | 6815 |
| Smad2 | GGATGAAGAAGCTTGGAGGG | 6816 |
| Smad2 | GTAGTGCTAGTGAGGCTTGC | 6817 |
| Smad3 | GGGAGAGAATTAACATTTCA | 6818 |
| Smad3 | GGAGGGCAGAGGACAGAAAG | 6819 |
| Smad3 | GCTGAGGTCTACTGAGCCTC | 6820 |
| Smad3 | GGCCATACCCAAAGAAACCT | 6821 |
| Smad3 | GTCTCTAGCAGCAAGTGGAA | 6822 |
| Smad3 | GCTTGGTTCACTGGGCCCAA | 6823 |
| Smad3 | GTGGCCAGAGCTGCTTTAGG | 6824 |
| Smad3 | GCAAGCAGGGCTGGGATCAT | 6825 |
| Smad3 | GGAGTGCAGCCAGCCCTTGA | 6826 |
| Smad3 | GGGATGAAGGTTTGCTTAGG | 6827 |
| Smad4 | GGACCACAGGGCATGAACAC | 6828 |
| Smad4 | GACAGCTCGGGATGAGCGAT | 6829 |
| Smad4 | GCTAAATAGGTTGTGCAGGC | 6830 |
| Smad4 | GGACACAGCTGGACCGAGTG | 6831 |
| Smad4 | GACCACATCCGGGTAATTTC | 6832 |
| Smad4 | GGCCAAACCCTGAAATTACC | 6833 |
| Smad4 | GTCAGCTAGAGGTCCTCCCA | 6834 |
| Smad4 | GGGAATGAGTCTTCTTTCCT | 6835 |
| Smad4 | GAGAAAGGAGCGCTGCGGGA | 6836 |
| Smad5 | GCGAGCCTGGAAGTGGCACT | 6837 |
| Smad5 | GCAAGACTTCTTTATGCCTC | 6838 |
| Smad5 | GGAATTACACTCCGGCCAGC | 6839 |
| Smad5 | GTCCAAGCTGACCGTTTGGA | 6840 |
| Smad5 | GAGATTAAATAAATGCCGTG | 6841 |
| Smad5 | GGAAATGTAATCAAGTACAA | 6842 |
| Smad5 | GGCATAAAGAAGTCTTGCTT | 6843 |
| Smad5 | GTTTCCTGGCTGACAACTGC | 6844 |
| Smad5 | GGGAGTTGTAAATCCATGCC | 6845 |
| Smad5 | GGTGCTCGAGCTGTCTTACA | 6846 |
| Smad6 | GGCATAAGGTAAATCCTCGA | 6847 |
| Smad6 | GCATCCAATTCAGGTTGTCA | 6848 |
| Smad6 | GTGAATATGAGTAGCTGTCC | 6849 |
| Smad6 | GAAGCCTGCTACCTTAAGCT | 6850 |
| Smad6 | GGCCTTGGCACTCTATATAA | 6851 |
| Smad6 | GTGGGTTCACCCAGCAGAGC | 6852 |
| Smad6 | GAATTTCTTTCATTGAGCTC | 6853 |
| Smad6 | GTGGTGTGCAAGTCCAGGAA | 6854 |
| Smad6 | GTGTTTGTTAGCGCGTGTGC | 6855 |
| Smad7 | GTCTAAATCGGGCCACTAAC | 6856 |
| Smad7 | GAGGGCACAGGCTAGTGTGG | 6857 |
| Smad7 | GACAGCAGTCAAGAAGACCA | 6858 |
| Smad7 | GCAGCATCCTGGAGGGAGGA | 6859 |
| Smad7 | GACATTTACACCGGCCAGGA | 6860 |
| Smad7 | GCTGGTGCTTTATGGTTCCC | 6861 |
| Smad7 | GGCGACAGCAGCAACAGCAG | 6862 |
| Smad7 | GTGTGTCTTGTGCACAGCTC | 6863 |
| Smad7 | GACACACATTTAGAGGGCTG | 6864 |
| Smad7 | GCAGTCAAGAAGACCAAGGA | 6865 |
| Smarca4 | GCTACTGCCTCTTAAACGCT | 6866 |
| Smarca4 | GAACTGAGCTGTGTGTGTTG | 6867 |
| Smarca4 | GAGGCATGAATCTACAGATT | 6868 |
| Smarca4 | GCTAATTACTGGGCCTCAGC | 6869 |
| Smarca4 | GTTCTGCAGGAAATGTGGCC | 6870 |
| Smarca4 | GCACGCGTACTAGTCCTTTG | 6871 |
| Smarca4 | GAGTTGCCCACTCAATAGAC | 6872 |
| Smarca4 | GTTCTATGCTCCAAGGCTAA | 6873 |
| Smarca4 | GAAATTAAAGTCCTCAGGCT | 6874 |
| Smarca4 | GGTCCAGATGGGAGATAGTA | 6875 |
| Snai1 | GTGTTGGAACGTTCACAGGG | 6876 |
| Snail | GGAGGCAGAGCTAGAAACTT | 6877 |
| Snai1 | GGCAGAGGTAGCAAGGACCA | 6878 |
| Snai1 | GCTGTATGGTCTTCTATTGT | 6879 |
| Snai1 | GCTGGCATGCCGCTTAGGAA | 6880 |
| Snai1 | GGGAGGTGTGATTTGATGAA | 6881 |
| Snai1 | GAACAGGCTTTCCTACCACG | 6882 |
| Snai1 | GCAGGTGTGAGGTTGTGAAC | 6883 |
| Snai1 | GCTGCTGACCTTTGGGCGCT | 6884 |
| Snai3 | GATGCAGTCTGTTTATGCCT | 6885 |
| Snai3 | GGAACTGGCCAGCGATCCCT | 6886 |
| Snai3 | GCCTGAGTGGTTAGCAACGA | 6887 |
| Snai3 | GTCTGGTGGGACAGTCTCTG | 6888 |
| Snai3 | GGGATCTCCAATTTCCTTCA | 6889 |
| Snai3 | GGAACTGCCAGTTTCATGAA | 6890 |
| Snai3 | GATGACATCCIGAAAGCATT | 6891 |
| Snai3 | GGCAGCGTAGGAGACAGTGG | 6892 |
| Snai3 | GAACTCTGCTCTTTCATCCA | 6893 |
| Snai3 | GCTCAGAATGAGGGTGGAGG | 6894 |
| Sox1 | GAAACCCAGCAGAGGTACTT | 6895 |
| Sox1 | GCAGAATAACAGCGGTGCGG | 6896 |
| Sox1 | GGCACAGAGTTGGCTGGCTG | 6897 |
| Sox1 | GAGGAAGAAAGAATCGCTGT | 6898 |
| Sox1 | GCTGACTTGCCCTAACACAG | 6899 |
| Sox1 | GAACTCGGGTTTGCGAGGGT | 6900 |
| Sox1 | GCTCCGAATGATTAACGATT | 6901 |
| Sox1 | GAATCTGTAAAGGCCTTTGC | 6902 |
| Sox1 | GAAGAACTTGTAGACTCTAA | 6903 |
| Sox1 | GTGCTTCGGGAGGTTGCTGG | 6904 |
| Sox10 | GTTGAGTGGCTAGGCGGAAC | 6905 |
| Sox10 | GGGTGTGAGTGTGTGTGTGG | 6906 |
| Sox10 | GTCCTTACCCGGTCCTAATG | 6907 |
| Sox10 | GGCATAGAGGAGTGCTGTGG | 6908 |
| Sox10 | GACACACTGGCCCAATTGTC | 6909 |
| Sox10 | GCCAAACCCAAGCTGAGTCC | 6910 |
| Sox10 | GTGTCTCTCACTTCCATGAA | 6911 |
| Sox10 | GAGATAGTCACATAGGGCAA | 6912 |
| Sox10 | GAGACACAGGAGGCTGAGGC | 6913 |
| Sox10 | GTTGTATGTGTACAGGGCAA | 6914 |
| Sox11 | GGCCTATGGAGTAGAAAGTG | 6915 |
| Sox11 | GAAAGAGGATCCCAAATAAG | 6916 |
| Sox11 | GCGGTTCGGAAAGGAGTTCA | 6917 |
| Sox11 | GACCGTTACTCCAGCCGAAC | 6918 |
| Sox11 | GGTTTCAGGACCGAGCTGCA | 6919 |
| Sox11 | GTGCAGTACACCAACCTGAA | 6920 |
| Sox11 | GGAAAGGAGTTCACGGATTC | 6921 |
| Sox11 | GTCGAAGCGCCACCTTCTGC | 6922 |
| Sox11 | GCGACCGGGCTCTAGAAAGA | 6923 |
| Sox12 | GGATGCTAGAGCCTGGGTGT | 6924 |
| Sox12 | GAGGTCACCTTCATGGCGCC | 6925 |
| Sox12 | GAGTGATCTGGAAGGCAGGC | 6926 |
| Sox12 | GCTGCACCTGGAGTTGAGTG | 6927 |
| Sox12 | GTCTCTTACTGGGACACTGA | 6928 |
| Sox12 | GATCCGGGCTGGAGTGAAGT | 6929 |
| Sox12 | GAGCCAGTTGTAGCACCGCC | 6930 |
| Sox12 | GCTGTTAGCATGGATTTCCA | 6931 |
| Sox12 | GCTGGACCCTGTGTGTAGTA | 6932 |
| Sox12 | GACCGCCAGACTAGCTAGAA | 6933 |
| Sox13 | GTCCTAAAGCAGGCTTGTGT | 6934 |
| Sox13 | GACACACGATTGCCACGTAT | 6935 |
| Sox13 | GTGATCCCTGGCATCTGcTT | 6936 |
| Sox13 | GCCAGGTCCTTGTGTGCTAC | 6937 |
| Sox13 | GGTGCACAGACATGCTGTCT | 6938 |
| Sox13 | GGGCTGGGAGGTGCTTTGTT | 6939 |
| Sox13 | GACACAGTGGCAGCCCTTTC | 6940 |
| Sox13 | GTACACTTCAGTTCCCAGGC | 6941 |
| Sox13 | GCTCGTACACTTCAGTTCCC | 6942 |
| Sox14 | GGGTCCAGGGAATGAGGTCT | 6943 |
| Sox14 | GGAGTGTGTTCCAGACTTAT | 6944 |
| Sox14 | GGCCGATGCGAAATGCCCTT | 6945 |
| Sox14 | GGACGTAACACAACTCGTGC | 6946 |
| Sox14 | GGTGCACAGTCTGCATTTGA | 6947 |
| Sox14 | GTGAAGAGTCCTAGTGGCAA | 6948 |
| Sox14 | GCGAAGTTCAAAGGCGAGGT | 6949 |
| Sox14 | GeCGCCTCCACCTGTAATCC | 6950 |
| Sox14 | GAGCTCTGGGCTTGCTGGCT | 6951 |
| Sox14 | GCTTCATGCGGGCTTCGCAG | 6952 |
| Sox15 | GTAGGGTGGACAAGAGGGAG | 6953 |
| Sox15 | GGCAGGTTGTATTTCTGGCC | 6954 |
| Sox15 | GGCCTCCGGTGGAACGTTAG | 6955 |
| Sox15 | GGAGCTGCTCTTATCTACGG | 6956 |
| Sox15 | GCTGGAGCTGCTCTTATCTA | 6997 |
| Sox15 | GCCTCCGGTGGAACGTTAGG | 6958 |
| Sox15 | GAGTTGGGTAGTTTGGTGAA | 6959 |
| Sox15 | GAACACTACCTCTCCGGTAA | 6960 |
| Sox15 | GGGTAGGGTGGACAAGAGGG | 6961 |
| Sox15 | GGATTCTCTTTCAGGACAGA | 6962 |
| Sox17 | GTCCTACCCAGTTTGCTCTC | 6963 |
| sox17 | GAGTCAGTAGTGATGGATTA | 6964 |
| Sox17 | GGTACATCCTTGGAATGTTA | 6965 |
| Sox17 | GGACTTGAATGTCCTTTAAC | 6966 |
| Sox17 | GTTTACTTCCTGCTTCGCCG | 6967 |
| Sox17 | GAGTCGCCAGCTGCTAGGGT | 6968 |
| Sox17 | GTCGATTGGCACCTTTCACC | 6969 |
| Sox17 | GGAGAGCAAGTTCATGAGGG | 6970 |
| Sox17 | GAGACAAATTGGAATTTACA | 6971 |
| Sox17 | GGCTCATTCCGCACACCGTT | 6972 |
| Sox18 | GTCCTGAAAGCATTTCACCT | 6973 |
| Sox18 | GAACAACTGGTACAGGAGGA | 6974 |
| Sox18 | GTTTCTGAACACTCTTGCCA | 6975 |
| Sox18 | GCTCTGGTGGCTGGATTTGG | 6976 |
| Sox18 | GCCCTACCATTCCAACTTTC | 6977 |
| Sox18 | GTCCCATCTGGAAGGAGGGT | 6978 |
| Sox18 | GGAATTCTGGGATCTCTCCA | 6979 |
| Sox18 | GAATAGGGTGCTGAACCAGA | 6980 |
| Sox18 | GCTACTTCCCTGGCTAAGTC | 6981 |
| Sox18 | GCTCCTCAGACTAAAGGATG | 6982 |
| Sox2 | GTGACAATAACAGCCAAGCC | 6983 |
| Sox2 | GCTGGCGACAAGGTTGGAAG | 6984 |
| Sox2 | GGCTGTGGGAGAATGGGCTG | 6985 |
| Sox2 | GGAAAGAAGCTCCCGAGTGC | 6986 |
| Sox2 | GACTGTCCAACTAGTATTTC | 6987 |
| Sox2 | GCCTTTGCACCCTTTGGATG | 6988 |
| Sox2 | GGCAGTTTCAGAGGAAACCT | 6989 |
| Sox2 | GGGTTGGGAGTTAGAAAGAG | 6990 |
| Sox2 | GATAAACAGGGCAGTTTGTA | 6991 |
| Sox2 | GCAGCCACATCTCAGAAACT | 6992 |
| Sox21 | GAGTCACACCTGGCCCTCCA | 6993 |
| Sox21 | GGCCTCAGTGGAGACTGTCC | 6994 |
| Sox21 | GTAGGTTATAGGAAAGGGAA | 6995 |
| Sox21 | GTGGATCCCACCATGAGGCT | 6996 |
| Sox21 | GGAAAGGAGAGCAATTATGA | 6997 |
| Sox21 | GCGAGGAAGAGGGTTGAGCC | 6998 |
| Sox21 | GAAGCTTTCGGGACTGGGAA | 6999 |
| Sox21 | GCTATATCACCTGAGATCGC | 7000 |
| Sox21 | GTGGGATCCACGTGGAATCG | 7001 |
| Sox3 | GACAAACAGCTAATCTGCTT | 7002 |
| Sox3 | GGAGCGGGTTTAGGATGCAA | 7003 |
| Sox3 | GGGCTCGGTGTTGATTGGCC | 7004 |
| Sox3 | GTCACCGCAGAGAAGCCAAG | 7005 |
| Sox3 | GAGACAGAAGCCGGGAGTAC | 7006 |
| Sox3 | GCCATGCCACTTGCTTGAGC | 7007 |
| Sox3 | GGTGTCTTAGTCTTCAGTGC | 7008 |
| Sox3 | GGTCTGCGCCCTGCAAACGT | 7009 |
| Sox3 | GAGCTTTCCAGGTGGGCCAT | 7010 |
| Sox4 | GATGTTCGAGAGACTAAGGT | 7011 |
| Sox4 | GAGTGTGTGATTATAAACCA | 7012 |
| Sox4 | GTCACACATTCAGAGTATTT | 7013 |
| Sox4 | GCTCTTTGAGACAAGGACTT | 7014 |
| Sox4 | GCATCGGGTTCCAAGCCAAT | 7015 |
| Sox4 | GTCTATGTTTCTCTTAGACC | 7016 |
| Sox4 | GCACGATGTTCGAGAGACTA | 7017 |
| Sox4 | GACAATGGGTAAGAAAGAGA | 7018 |
| Sox4 | GTAATAGTATATGCCATCAA | 7019 |
| SoX5 | GGACCTAATCAAACTGCGGT | 7020 |
| Sox5 | GGTAAAGCGAATCATAGGAG | 7021 |
| Sox5 | GAGTGTGCGGCTGTGCAGAG | 7022 |
| Sox5 | GCAATCCTGAAGGTCAGCAC | 7023 |
| Sox5 | GCTCACAGCATCTCACCTTA | 7024 |
| Sox5 | GTTAATGCTCACAGTTTGAT | 7025 |
| Sox5 | GTGAGTGACAGCCTGTTTAC | 7026 |
| Sox5 | GGAAGGTGGAAGGAGTGGAG | 7027 |
| Sox5 | GGACTGGTCAGGCCATCTTC | 7028 |
| Sox5 | GGTCAGGCCATCTTCTGGTT | 7029 |
| Sox6 | GTGTTACATACCTCTGAGTT | 7030 |
| Sox6 | GGAGTGGGAGAAATGGGCTC | 7031 |
| Sox6 | GCCTACAAGAAACTGTATAC | 7032 |
| Sox6 | GGCCCTTGTAGATGGATCGT | 7033 |
| Sox6 | GAAACAGCTGGGCTGCACAC | 7034 |
| Sox6 | GTTGTGCCTTACTCCGGAGG | 7035 |
| Sox6 | GCCACTACGACCCATCATGC | 7036 |
| Sox6 | GATCAGCTCATCTATAGCTG | 7037 |
| Sox6 | GCTATTTAGCTGAGAACTCT | 7038 |
| Sox6 | GGTGGCAAGGGTACTTGGGT | 7039 |
| Sox7 | GCCATCTGTAGGCTGGAACC | 7040 |
| Sox7 | GGACGACAATGGATCACAAG | 7041 |
| Sox7 | GGCCCTTATTTATCAGCTTC | 7042 |
| Sox7 | GTGGCTGCCCACGTTTACTG | 7043 |
| Sox7 | GAGAGGCCAGCGCCTGTTTG | 7044 |
| Sox7 | GTGAGATCAGCCTTATCGCC | 7045 |
| Sox7 | GGAAAGGTCTTGGGAGATAC | 7046 |
| Sox7 | GCTTTCTGAGAAAGAGGAAC | 7047 |
| Sox7 | GATAAGGCTGATCTCACAGG | 7048 |
| Sox7 | GCAGCGATCACCGGCTTTAA | 7049 |
| Sox8 | GAGTTACCAGGGTCACCTGG | 2050 |
| Sox8 | GGAATGCCCAATACAAACTC | 7051 |
| Sox8 | GCTAGAAAGAACGTTATTCA | 7052 |
| Sox8 | GTCTGGGTGGCATAGAGCTG | 7053 |
| Sox8 | GGCTGAGGATGTGAACCAAT | 7054 |
| Sox8 | GAAAGAACGTTATTCAGGGT | 7055 |
| Sox8 | GCTAGACAGAGGTGGGAGGG | 7056 |
| Sox8 | GTCCTTCCGGGTATGACCTG | 7057 |
| Sox8 | GTTCTCTGGGCAGCTCTTCC | 7058 |
| Sox8 | GGAGAAGCAGGCCAAGGCTG | 7059 |
| Sox9 | GGACAGACTTGGCCTGATCT | 7060 |
| Sox9 | GCTGGCATTTCTTCCAGAAC | 7061 |
| Sox9 | GGTTGGGTGACGAGACAGGA | 7062 |
| Sox9 | GTAGACGCACTTCTATGTTC | 7063 |
| Sox9 | GTCCACACTTAGCAAATTAG | 7064 |
| Sox9 | GGAGTGGACTTTACCTGTTC | 7065 |
| Sox9 | GAGGGCGAAGTTTGCAAAGG | 7066 |
| Sox9 | GCACACAGGTGGGCGTTCTG | 7067 |
| Sox9 | GTCCTCTTAGACCTGCACAC | 7068 |
| Sox9 | GAGGATTGTGGCTCCGGGTT | 7069 |
| Sp1 | GTGCTAAATGCCTATTTAAC | 7070 |
| Sp1 | GAGTTGGTTTAGCAGGTCTG | 7071 |
| Sp1 | GTGGAGGCTGGAACTTGGAA | 7072 |
| Sp1 | GTCGCCATGTTGGCCCTCCT | 7073 |
| Sp1 | GCCCAATGAGGGAGGGTGAA | 7074 |
| Sp1 | GGAGAAAGAAGGCGAAATGG | 7075 |
| Sp1 | GCTCCGTGAGCGGTAGGGAT | 7076 |
| Sp1 | GAAATAGGCCGGAATGGGAT | 7077 |
| Sp1 | GGGCCTTGCAGAGGAAAGGC | 7078 |
| Sp100 | GTTCCATTGTCTAGAGTCCT | 7079 |
| Sp100 | GGATGCTTGGATAGTCTGAG | 7080 |
| Sp100 | GGATGGATGACCACTATAAA | 7081 |
| Sp100 | GTTCTGACAAAGTGTAGAAT | 7082 |
| Sp100 | GTAGAGATGGGAGCCGACCT | 7083 |
| Sp100 | GAACTGAAATTGCTGGTGAT | 7084 |
| Sp100 | GAGTGGGTAGAAAGCTCAGG | 7085 |
| Sp100 | GATCTCTTCTGTCTTTCAGA | 7086 |
| Sp100 | GCTACAGCATCGCTTCCTGC | 7087 |
| Sp2 | GGGCTGACTATCCTGCTGGG | 7088 |
| Sp2 | GAGATGTATAAGCTCTTTAC | 7089 |
| Sp2 | GCCCATACATTCTGTTCCCA | 7090 |
| Sp2 | GTCTGAAGCTGAGAGGATCA | 7091 |
| Sp2 | GGAAGCATCTAGAGTGACGT | 7092 |
| Sp2 | GAGCGCATCGCCTTCACCTC | 7093 |
| Sp2 | GCTTGACAGGCACCACAGGT | 7094 |
| Sp2 | GAGAAAGCTAAACCTACCTG | 7095 |
| Sp2 | GAGACACATACATCCCTGCT | 7096 |
| Sp3 | GTGTTTAGGACAGCTCAGGC | 7097 |
| Sp3 | GACTAGCTAGAAACGTTATA | 7098 |
| Sp3 | GTGTCACAGTGACTCAACTG | 7099 |
| Sp3 | GCTGCTGCTACTGAGCAAAC | 7100 |
| Sp3 | GCATTGAGGATGTCAAAGGA | 7101 |
| Sp3 | GCTCTAAGTGCCCGCCTCCA | 7102 |
| Sp3 | GGCAAATGAGAGCCGGGAAG | 7103 |
| Sp3 | GACTTTCTTGGTTAAGAAGC | 7104 |
| Sp4 | GACAACCTTGTGAGACCTCT | 7105 |
| Sp4 | GGAACTCTCCAATTCATGCC | 7106 |
| Sp4 | GAGGAAGGCGGTGCCTCAAT | 7107 |
| Sp4 | GTTGTCTAAAGAGAACCACA | 7108 |
| Sp4 | GAGCTGACCCACATGCAGCC | 7109 |
| Sp4 | GCAAAGAAAGCAGGGCGAAG | 7110 |
| Sp4 | GGCTCGGCTCTCATTGGATG | 7111 |
| Sp4 | GTACTGGTATCCAGGAAACA | 7112 |
| Sp4 | GCGTTCCACATTTATTGACG | 7113 |
| Sp4 | GTGGTCATTGTACTTCACAT | 7114 |
| Sp6 | GGATCTCTGGAAACCAGGAG | 7115 |
| Sp6 | GATGCCATGGAAATCTAACC | 7116 |
| Sp6 | GCAGGAGAGAATAAAGTGAT | 7117 |
| Sp6 | GGTTTATTCTGTTCCTAGCA | 7118 |
| Sp6 | GGTTGGGCGGGCATCTGAAA | 7119 |
| Sp6 | GCGCACTGGGATCAGAGGGT | 7120 |
| Sp6 | GGAAACTGAGGAAGACATTG | 7121 |
| Sp6 | GTGAGGTAGGATGGGCTCCA | 7122 |
| Sp6 | GCTTAGTGCTGGGTGTGGGC | 7123 |
| Sp6 | GCTCTTAACTCAGAAGTGGT | 7124 |
| Spdef | GCCTGATGCCCTCAAAGGCC | 7125 |
| Spdef | GAACGCAACAGATGTGTCCT | 7126 |
| Spdef | GAAGAACAGAGACAAATGGA | 7127 |
| Spdef | GTAGTCAGCCCAGCCTGCTG | 7128 |
| Spdef | GGGAAAGCCACCTGACATTC | 7129 |
| Spdef | GCTTCCTGGAGGTGGTGCAG | 7130 |
| Spdef | GAGGGATGGACAGAGAGGGT | 7131 |
| Spdef | GCCCTCAAAGGCCCGGGAAA | 7132 |
| Spdef | GCCTTCCTGGATGTGTGCTA | 7133 |
| Spdef | GCTGCCCAAATGTGCCTTCC | 7134 |
| Spi1 | GATGCTGGCCTCAGGATGAC | 7135 |
| Spi1 | GAGTTTCTGTTTGTTCTAAG | 7136 |
| Spi1 | GAGTTCCTAGTGAAGGTCCA | 7137 |
| Spi1 | GAGATGTGCAGACAGATTGT | 7138 |
| Spi1 | GGAGGTCTTGGAGCCAGTGG | 7139 |
| Spi1 | GATGCCAGGCTGCATAGCAA | 7140 |
| Spi1 | GAAGGCTGAGAAGCCCAGCT | 7141 |
| Spi1 | GGAGGAAGGAGGGAAGGCTA | 7142 |
| Spi1 | GCCAAACAGACCATGGAACA | 7143 |
| Spi1 | GAAGGGTCAGAGCAAGGCCA | 7144 |
| Spib | GCGCAAGGACCTGGAAGACC | 7145 |
| Spib | GTTCTGTCAGCCACGGGAGT | 7146 |
| Spib | GAGCTACACACTGTATCTGC | 7147 |
| Spib | GGCTGTGCTCCAGCACAAAC | 7148 |
| Spib | GTGTGGTCACCGCCTAGAGG | 7149 |
| Spib | GGCTCAAAGATGCGCAAGAG | 7150 |
| Spib | GTTGCCCTGAGGTGTGCTAG | 7151 |
| Spib | GACTGTGCTCACCAGCAAGG | 7152 |
| Spib | GCTGTATATCAGCTGTCACC | 7153 |
| Spib | GTTAAGTGCAGAGGCGGGAA | 7154 |
| Spz1 | GAGCCTAGGAGAGAAGAGAG | 7155 |
| Spz1 | GTTGTGGGACAGGAATCTAA | 7156 |
| Spz1 | GGCTGTGGTGTCTGAGTTGT | 7157 |
| Spz1 | GCTCTTAGAATGAAGAGCCT | 7158 |
| Spz1 | GGGAGGAGTAAGGTTGGCTG | 7159 |
| Spz1 | GGAAGTGTTGCTCCAGCTGT | 7160 |
| Spz1 | GTCACTCTCATACTCTTTCT | 7161 |
| Spz1 | GGAGGACTGAGGATACAGTT | 7162 |
| Spz1 | GATTCCCAAGTGGAGGACTG | 7163 |
| Spz1 | GACAACTAGGCTCAACGTCA | 7164 |
| Srebf1 | GAGAATGCTGGCCCTAGATG | 7165 |
| Srebf1 | GTTCCTAAGTCACAGGGCCC | 7166 |
| Srebf1 | GAGGACCTGAGCCCAGCTAC | 7167 |
| Srebf1 | GACCTCTGAGTCCTTCTGGC | 7168 |
| Srebf1 | GCTCCACAGATTGGTTTACT | 7169 |
| Srebf1 | GTCTGCAGTGCTTAAAGGGT | 7170 |
| Srebf1 | GCTTCTTCTGTATCAGGCCA | 7171 |
| Srebf1 | GGAACAGGTAAAGCAAGGGA | 7172 |
| Srebf1 | GGACTACTCAACTGCAAGCA | 7173 |
| Srebf1 | GTCCTCTCTGCTCCAATGGT | 7174 |
| Srebf2 | GGACCTAAGTGTATACTGAG | 7175 |
| Srebf2 | GGATGGGATAAGTGTGACTT | 7176 |
| Srebf2 | GAATAACAACCTAGCTCCTG | 7177 |
| Srebf2 | GGAGCTAGGTGCCAGCTGAA | 7178 |
| Srebf2 | GAGGTGTGGGACCAGTGTGG | 7179 |
| Srebf2 | GCTCTCGACAAAGTTGCTCC | 7180 |
| Srebf2 | GTCGAGAGCCCGGAAATAGA | 7181 |
| Srebf2 | GATGGGATAAGTGTGACTTA | 7182 |
| Srebf2 | GACTTGGAAGTTTAGGAGAC | 7183 |
| Srebf2 | GACATATCTCTGGAGGGCAG | 7184 |
| Srf | GCACAGGCCTGAAAGTACAG | 7185 |
| Srf | GGCAGACACACATCTGGAGG | 7186 |
| Srf | GACCTCGCAGCCAGACTTGT | 7187 |
| Srf | GTGTTACAAAGCCCAGGTTA | 7188 |
| Srf | GACTCTCAGACCCTTAACCT | 7189 |
| Srf | GTGGCAAGTCACAAACTTCC | 7190 |
| Srf | GAGGACTGCAGGGCAGAAGA | 7191 |
| Srf | GTTGAAACTATCCTAGAGGA | 7192 |
| Srf | GTTCTAAGTCCAGATTTAAA | 7193 |
| Ssrp1 | GCTGGCTTTAATGTAGACTT | 7194 |
| Ssrp1 | GCCTGAGATGCAGCTGGCTA | 7195 |
| Ssrp1 | GGTATGTGCTCCTAAGAGGC | 7196 |
| Ssrp1 | GGCTTATCCTGTCTTTGTGT | 7197 |
| Ssrp1 | GACTTTGGCAAACAATGGCT | 7198 |
| Ssrp1 | GCCCTCCTGCACACATACAA | 7199 |
| Ssrp1 | GCTGCATTAAATTCAAGTGG | 7200 |
| Ssrp1 | GACTCTAAAGACTCAATGGA | 7201 |
| Ssrp1 | GCTTGCTATGGAATCCCACG | 7202 |
| Ssrp1 | GGAAGACCTGCCCAAGAGAA | 7203 |
| Stat1 | GTTCTGTGATGCCTTTGTGA | 7204 |
| Stat1 | GACAGTCATCAAAGGCACAA | 7205 |
| Stat1 | GTGCGGTGCAAACCGCAGAC | 7206 |
| Stat1 | GACACTTGGTCCTCGAGCCT | 7207 |
| Stat1 | GGCCAATCTCTGCCGCTGAT | 7208 |
| Stat1 | GTTCTCTCTGTGTTCTGCCT | 7209 |
| Stat1 | GGCTTGCGCAAGCTCAGTCT | 7210 |
| Stat1 | GGCTCGAGGACCAAGTGTCC | 7211 |
| Stat1 | GGAGCAGCTGCACCATTTCT | 7212 |
| Stat1 | GACTAAATGGGCAACGTCTA | 7213 |
| Stat2 | GGAACTACCGAAGCTACCCA | 7214 |
| Stat2 | GGATTGACATCAGGATGAGT | 7215 |
| Stat2 | GGATGCCACTTCACACGGAG | 7216 |
| Stat2 | GGTTGCCTCTCCGTGTGAAG | 7217 |
| Stat2 | GAGCTGCAGAGCAGAGGACA | 7218 |
| Stat2 | GAAGAGTGGACACACAGACC | 7219 |
| Stat2 | GGCTAAGAGCTGCAGAGCAG | 7220 |
| Stat2 | GCTTATGTTCTGTTTAGCAA | 7221 |
| Stat2 | GGGAAAGGAAACTGAAACCA | 7222 |
| Stat3 | GAGCTGCAGTGTAGACAGGG | 7223 |
| Stat3 | GGAGTGGATCACCCAGGTAA | 7224 |
| Stat3 | GAGTGGATCACCCAGGTAAT | 7275 |
| Stat3 | GTTATATATACACCTAGGGA | 7226 |
| Stat3 | GTGGCAATCAGCCACTTAGG | 7227 |
| Stat3 | GTGGGAAAGTCAGGAAGAAC | 7228 |
| Stat3 | GGCCATTCCTTAATTATGCA | 7229 |
| Stat3 | GGAGAGGCCATAGAATCCAC | 7230 |
| Stat3 | GTAATTACTAGATTGCGTGG | 7231 |
| Stat3 | GCCAGAACCATGCTCTTCCT | 7232 |
| Stat3 | GCTCCAGCAGGTTCAGCTCC | 7233 |
| Stat3 | GCACCTATGACAAAGGGAAG | 7234 |
| Stat3 | GTCTAGGATTTCACTGTGTG | 7235 |
| Stat3 | GACTTCACCAAGAACTTTCC | 7236 |
| Stat3 | GGCTCAGACTCACTCCTTAT | 7237 |
| Stat3 | GAGCACCTATGACAAAGGGA | 7238 |
| Stat3 | GAGTCTCGATCTGGTGGCTT | 7239 |
| Stat3 | GCCATTCTGGACAGCTTAGG | 7240 |
| Stat5a | GGCTTCAGTGTACCTGGGCT | 7241 |
| Stat5a | GGAGATAGGGCAGAAGAAAC | 7242 |
| Stat5a | GACCTTTCTAGGTCACTGGA | 7243 |
| Stat5a | GTCAATGCCTGGAAGTGGGT | 7244 |
| Stat5a | GAGAGAGCCAGAAGCAAGGC | 7245 |
| Stat5a | GCCCATTGCCTCATGGTAGG | 7246 |
| Stat5a | GCATGGGCAGTACCAAAGGA | 7247 |
| Stat5a | GGGTTTGCAAGGAGGATCAG | 7248 |
| Stat5a | GGCCTGCAAAGCACGTGGTA | 7249 |
| Stat5a | GCAGGGAGCCAGCTACCTTT | 7250 |
| Stat5b | GCACTTCTGTATCCCAAGGC | 7251 |
| Stat5b | GGGCTCTCAGATTCCCTAAG | 7252 |
| Stat5b | GTGTTTGGAGCCACAAAGGA | 7253 |
| Stat5b | GGGCAATCCACTGATCCAGT | 7254 |
| Stat5b | GGACCATACAGCTTCTATGT | 7255 |
| Stat5b | GAGGTGATAGCTTACAGGTA | 7256 |
| Stat5b | GTTCTTCAACACAAGAGGTA | 7257 |
| Stat5b | GGGAGTGACAGGTTTATCCA | 7258 |
| Stat5b | GCCTCCTTTCGTCATGATCG | 7259 |
| Stat5b | GAGCCTTCAAGTACAACTGG | 7260 |
| Stat6 | GGGAATGGATCAGTGCTAAG | 7261 |
| Stat6 | GGAAGTGTGAGTCCAAGAAC | 7262 |
| Stat6 | GCTCCATTGAACCACACTGG | 7263 |
| Stat6 | GTGGGCACCTGGAAGCACAT | 7264 |
| Stat6 | GAACCCTTAGCTCAGAATTC | 7265 |
| Stat6 | GTGCAAACTTAGATCCACCC | 7266 |
| Stat6 | GAGGGTAAGTTGTGAGGGTA | 7267 |
| Stat6 | GATACTGTAGGGAGGAAGTG | 7268 |
| Stat6 | GATGCACATGCGTGAGTTCA | 7269 |
| Stat6 | GCAGAGTGGCTTAAGCTGTG | 7270 |
| Stra13 | GAATAACATTGGCCTCCTGG | 7271 |
| Stra13 | GGCTTAAGGCATGGTGGCTC | 7272 |
| Stra13 | GCGTTCCACGTTCATTGGTT | 7273 |
| Stra13 | GTTGGGATGTGGGAGGGTTC | 7274 |
| Stra13 | GACCTCACCCAGCTGTTGGA | 7275 |
| Stra13 | GATCAGTCACAAGGGAGCAG | 7276 |
| Stra13 | GGCTCTGACAGCCATCAGGT | 7277 |
| Stra13 | GTTCAGGGCTTAAGGCATGG | 7278 |
| Stra13 | GCGTGAGGCTACAGGAAGGG | 7279 |
| Stra13 | GTAGAAAGTAAATGTGGTAG | 7280 |
| Sub1 | GTGGCCTTCGTGCCATTGGG | 7281 |
| Sub1 | GTAGACAGGAGTCACGGTGG | 7282 |
| Sub1 | GGATTTCCTCCGCGAGACTT | 7283 |
| Sub1 | GTACTTAGCTCCTGTATTCT | 7284 |
| Sub1 | GGCACGAAGGCCACGTGAAG | 7285 |
| Sub1 | GGCCCTTTCCAGGGCCTTAA | 7286 |
| Sub1 | GCTATAATAGTCTCCGTGCC | 7287 |
| Sub1 | GAGGCGGAACACCAAGTCCA | 7288 |
| Sub1 | GGAGTAGACAGGAGTCACGG | 7289 |
| Sub1 | GCTATAGGCTGCCCTGGAGG | 7290 |
| Suz12 | GAAGCTCTCAAGGCGAGAAA | 7291 |
| Suz12 | GCTCAGTCTCATCTCCACTG | 7292 |
| Suz12 | GAAAGGAGAAATGCACCTAA | 7293 |
| Suz12 | GCGGGTGACTGAGAAACTGA | 7294 |
| Suz12 | GATTTCGGCCATGGGTGGCT | 7295 |
| Suz12 | GCCAGACAAGACCAAGCTAG | 7296 |
| Suz12 | GCCTCTTTGAACTGAATTCG | 7297 |
| Suz12 | GTCAGGGTTCAGTTGTAGGG | 7298 |
| Suz12 | GCAACAACCTGTCCAATCAA | 7299 |
| T | GAACCTCTGCCGGGAGAGTG | 7300 |
| T | GTGATTCTCTTTCACAGTCG | 7301 |
| T | GCCTGAGACTTCCTGGAACT | 7302 |
| T | GAGTCTCCCTGGGAAGTCTT | 7303 |
| T | GCGTTTAACCTCTGGCGTGA | 7304 |
| T | GGTGCTCATTGCAGGAGGGT | 7305 |
| T | GGAGCACCGAGATCGGGATG | 7306 |
| T | GCACCAGCCAGTTTGTGTTG | 7307 |
| T | GGAATAAATCTCGGTGGAGG | 7308 |
| T | GGGCAGAGGAGGGTAGTCTA | 7309 |
| Tal1 | GGTGTGATCCTCACCCTGTG | 7310 |
| Tal1 | GTCGGGTTGTTTGTAGGGAG | 7311 |
| Tal1 | GGGTTCCTACAATGTACCTA | 7312 |
| Tal1 | GCCACCTTAGCTGGACAAGA | 7313 |
| Tal1 | GGAAAGACGGAGGAAACGGA | 7314 |
| Tal1 | GATAAGCGCCTCGGTCATTA | 7315 |
| Tal1 | GAATGTTAAAGGAAAGTAGG | 7316 |
| Tal1 | GAAACGGACGGGCAATTCCA | 7317 |
| Tal1 | GAGAGATCGAGGCGCTGGTG | 7318 |
| Tal1 | GAAGTGGCGTCGGTCTGCTT | 7319 |
| Tal2 | GATGGACATGTATTCAATAT | 7320 |
| Tal2 | GCGGTGTCCTATAAAGGCTG | 7321 |
| Tal2 | GGAACAGTTAAGTACAGCTA | 7322 |
| Tal2 | GATTAAAGTAAGGAGTCCTA | 7323 |
| Tal2 | GGACATGGTTATTTCAGGGA | 7324 |
| Tal2 | GTCATGGGCCATCAGGTGGT | 7325 |
| Tal2 | GTCTCTGCCACAGCCTTTAT | 7326 |
| Tal2 | GGTAGCATTGGTCTCTCCCA | 7327 |
| Tal2 | GAAAGATACAGGAGAGAAGA | 7328 |
| Tal2 | GCCTAGAACCTTGGTGCAGA | 7329 |
| Taz | GTTTCCAGACCCACCCAAAG | 7330 |
| Taz | GCCTGTAGACACTAGAATTA | 7331 |
| Taz | GGGAGGAACTTCAGAAGGAA | 7332 |
| Taz | GAATCCTGCGGGTAGGGAAA | 7333 |
| Taz | GGTATGAGAATCCTGCGGGT | 7334 |
| Taz | GCGCAGTTGGGTGTGTGTGG | 7335 |
| Taz | GTGCATAAGGTCCTTTGCTT | 7336 |
| Taz | GGTTTCCAGACCCACCCAAA | 7337 |
| Taz | GTATGAGAATCCTGCGGGTA | 7338 |
| Taz | GGTTTCCAGACACACCCAAA | 7339 |
| Tbp | GAGTAGCTGTTTCTGTCGCT | 7340 |
| Tbp | GAGCTGGTGTGAATTAGAAC | 7341 |
| Tbp | GTGCCGTTTGCTCCAGCAAC | 7342 |
| Tbp | GGCGTTCGGTGGATCGAGTC | 7343 |
| Tbp | GCCCAGCACTCAGTTGTGCA | 7344 |
| Tbp | GGTCCGACTGCCTAAGGCTG | 7345 |
| Tbp | GGAAGATTGAGGTGGGAGCC | 7346 |
| Tbp | GTTTGAGGAGATACAACCCA | 7347 |
| Tbx1 | GAGTCCCACGTGAGGATGTA | 7348 |
| Tbx1 | GCACCTGGGTAAGAGAGCTC | 7349 |
| Tbx1 | GGACTAAGAGGTGTAAGCTC | 7350 |
| Tbx1 | GTTGCTGCTACAGCCCGGGA | 7351 |
| Tbx1 | GAGAAATTCAGACCGCATGG | 7352 |
| Tbx1 | GAAGGTCTTATACTAGGGTA | 7353 |
| Tbx1 | GGGAACTTCAGGAATTCTAC | 7354 |
| Tbx1 | GGTACTGTCAGGCAGAGGTG | 7355 |
| Tbx1 | GCAACTAAGTGGAAGGATCA | 7356 |
| T1x1 | GTTAGGCCTTTCGTGTGGGC | 7357 |
| Tbx15 | GCGGTTGTCCCGGCAGATTC | 7358 |
| Tbx15 | GAGAGTTAAGAGACCTGCAT | 7359 |
| Tbx15 | GGTTGTTTGGAATAAGAGCC | 7360 |
| Tbx15 | GCCAGGTTTGGACTGAGAAA | 7361 |
| Tbx15 | GCTGCTGAGGGAAGGAGGAA | 7362 |
| Tbx15 | GGTGTTGATGCTTACCTTGA | 7363 |
| Tbx15 | GGTTATCTGTGGTGAATGAA | 7364 |
| Tbx15 | GTTGTTGCTTCCAGCAGCAG | 7365 |
| Tbx15 | GCCAACAGTTCACCAGGATG | 7366 |
| Tbx18 | GCTTTCTTCTGGCTTCTCCT | 7367 |
| Tbx18 | GTGACGAATGCACTGCCACT | 7368 |
| Tbx18 | GGAGTGTGTTCCTATAACTC | 7369 |
| Tbx18 | GGTCATTCTCTCCATACAGT | 7370 |
| Tbx18 | GCTCCATTGGACCATCTATG | 7371 |
| Tbx18 | GGGTTCAGCTTTCTAGAGAC | 7372 |
| Tbx18 | GAGAAACCTGCAGTTCCTTC | 7373 |
| Tbx18 | GGAGCTGTCCATCACCGAAA | 7374 |
| Tbx18 | GGCTGGGCGCTCTAGCTCAA | 7375 |
| Tbx2 | GAGGGTGGGAGTATCCACTG | 7376 |
| Tbx2 | GATTCTCCACACGCGCCAGA | 7377 |
| Tbx2 | GAATGGATGCGGGAAGGCTG | 7378 |
| Tbx2 | GACCGATCTGACCCGCCGTA | 7379 |
| Tbx2 | GCACTTCAGAGGGAGGCTGC | 7380 |
| Tbx2 | GATCATACACTTGCCTGTTT | 7381 |
| Tbx2 | GGTCCATGCACTTCAGAGGG | 7382 |
| Tbx2 | GAGCCCTCATAGAATGGATG | 7383 |
| Tbx2 | GGCGGGCAAATCAGGAGGCT | 7384 |
| Tbx2 | GCCATGGCAGAACCCTGATG | 7385 |
| Tbx20 | GCTGCATCGCTTTGCTCCTG | 7386 |
| Tbx20 | GCGCCTTAATTTGCTGGCGG | 7387 |
| Tbx20 | GTTTGTTTCCCTTCTAGTCT | 7388 |
| Tbx20 | GATGAGGAATTTGCTCTACT | 7389 |
| Tbx20 | GCAGATAGATGGTTCCGTGT | 7390 |
| Tbx20 | GGAGTCATCGTCGTTACTTA | 7391 |
| Tbx20 | GGTTTGTTTCCCTTCTAGTC | 7392 |
| Tbx20 | GAAGTGGCCTGAAGCAAGGA | 7393 |
| Tbx20 | GCTGACAGGCTTGTGTGTTC | 7394 |
| Tbx20 | GGGAATGACTGGGACATGGT | 7395 |
| Tbx22 | GTGCCAGCAGTGTCAGTCCT | 7396 |
| Tbx22 | GAGGAGCAGTTCGTGGAGGA | 7397 |
| Tbx22 | GGCTGTTGACTGTCCCTAGA | 7398 |
| Tbx22 | GGTGGTGGGTTGCCAGCCTA | 7399 |
| Tbx22 | GGATGAGGACATCTGTGGAG | 7400 |
| Tbx22 | GCCAGTTGTTGGCTTCTGAC | 7401 |
| Tbx22 | GCTGCTTGAGTGTACTTACC | 7402 |
| Tbx22 | GGAGATGCAGCCTGAGCTGC | 7403 |
| Tbx22 | GGGACATTAATGCTCTGGTG | 7404 |
| Tbx22 | GCCATTTAACATCAAGTTCC | 7405 |
| Tbx3 | GGAGAATCTACAAGGTTAGC | 7406 |
| Tbx3 | GTGTTCTGCCAGGGAGGCCA | 7407 |
| Tbx3 | GAGGCGCCTTCCCGTTTCTC | 7408 |
| Tbx3 | GCGAGAGAATATTTCTGCTA | 7409 |
| Tbx3 | GCGAGGGAGGATCAAGAAGA | 7410 |
| Tbx3 | GGGAATTCTAGAGGCGGAGG | 7411 |
| Tbx3 | GTTTATCACCCACCAGGAGG | 7412 |
| Tbx3 | GCAGTTCCTTCTCCCAGGTA | 7413 |
| Tbx3 | GCCCTGAGCTTTCCCTGGTG | 7414 |
| Tbx3 | GTCTCCGCTCATCCTAGGGT | 7415 |
| Tbx4 | GAGGGCAGCCAGGATATCTG | 7416 |
| Tbx4 | GAGGAAAGGGATGGTCGGAA | 7417 |
| Tbx4 | GTAACCGTGAACTCCGTGCC | 7418 |
| Tbx4 | GTGTCATTAGGAACTTCCTC | 7419 |
| Tbx4 | GACACATTGATGAGGATTGC | 7420 |
| Tbx4 | GCTTGTGCGAATGTGAGGAC | 7421 |
| Tbx4 | GCTAGGATAAGCTTCCTCCA | 7422 |
| Tbx4 | GTGTGTGCTCTCTTAAGGGC | 7423 |
| Tbx4 | GGAGACCTCCGTAGAGCAGC | 7424 |
| Tbx4 | GGGCATACTCTGAAACACCA | 7425 |
| Tbx5 | GCGACTATCTCACCAGCCGC | 7426 |
| Tbx5 | GGATGCAATGGGTCCCAGAG | 7427 |
| Tbx5 | GAACCAAGACTGGATGCATT | 7428 |
| Tbx5 | GGAAGGAAGTGTTTCTGGCT | 7429 |
| Tbx5 | GGGCCTGCTGATTATTTATG | 7430 |
| Tbx5 | GAGGGAAACAGAATGTGATT | 7431 |
| Tbx5 | GGCAGGCCTAGCTTATTGCC | 7432 |
| Tbx5 | GGACAATGAGTCTGAAGTGG | 7433 |
| Tbx5 | GATGTCAAGGCAGCTAGTCC | 7434 |
| Tbx6 | GGGACGCAGTTTGGCGCTTC | 7435 |
| Tbx6 | GTTTATCTTGTGGGAGGGCC | 7436 |
| Tbx6 | GGGAATTGTAGTTCAGACTG | 7437 |
| Tbx6 | GGGTCACCTTCCAGAAAGTC | 7438 |
| Tbx6 | GCTGTGATCTCTGGTTTGGA | 7439 |
| Tbx6 | GCTAAGACAGGGACGCAGTT | 7440 |
| Tbx6 | GGGAGCATAAAGCCACTACC | 7441 |
| Tbx6 | GCTCACATGGAATACAGAGA | 7442 |
| Tbx6 | GCAACCTGTGCTGGGATCCC | 7443 |
| Tbx6 | GCCTTTACGTGCGACTGGCG | 7444 |
| Tceb2 | GCCACAAAGCATGGCTGAAT | 7445 |
| Tceb2 | GGAAGGTGAGCTGTTGACCC | 7446 |
| Tceb2 | GACTTCTGCTGTAGTTATTA | 7447 |
| Tceb2 | GCTCTTGCAGGGAATGTGAG | 7448 |
| Tceb2 | GGCTTCCGGATCGCTTAAGA | 7449 |
| Tceb2 | GAGTAGTGTTAGGCAAGGTA | 7450 |
| Tceb2 | GTCCTCTCCCTCCAGGAACC | 7451 |
| Tceb2 | GGATATTGTCCTCTCCCTCC | 7452 |
| Tcf12 | GCGCAGTGAGCTTGAGGAGA | 7453 |
| Tcf12 | GGTGAGCAAGCTGATGAGCG | 7454 |
| Tcf12 | GTATATTGCATAACCCAAAG | 7455 |
| Tcf12 | GAGGAGGTTTAGGAACTGCC | 7456 |
| Tcf12 | GGGTTTGGTTATCCGTAATT | 7457 |
| Tcf12 | GAGCACAGAGAAGACCAGCC | 7458 |
| Tcf12 | GAGATTGTACCACAGAAATA | 7459 |
| Tcf12 | GGGATTTGTTGGCAGGTCGG | 7460 |
| Tcf12 | GGAAGTTAAGGTTTACTTGA | 7461 |
| Tcf15 | GGGATATGCTCACTTTGGGA | 7462 |
| Tcf15 | GGTCGTCGCCTTATAGCCGG | 7463 |
| Tcf15 | GAAGTGACAGGATCAGCTAT | 7464 |
| Tef15 | GTAGTTATTAAGTGACTGAA | 7465 |
| Tcf15 | GCTGTCCAGGAGCGCAGATC | 7466 |
| Tcf15 | GACAGGATCAGCTATAGGTA | 7467 |
| Tcf15 | GAAGACATCTTCCAGCTCCA | 7468 |
| Tcf15 | GCTCAGTTCAAGGCCAGCAG | 7469 |
| Tcf15 | GGAGACCACTCAGCAAGAGA | 7470 |
| Tcf15 | GATATGATGTGAGGGCTGGC | 7471 |
| Tcf3 | GGTGTGATGGTAATCTTTGT | 7472 |
| Tcf3 | GTGAAGACTGAGCAGAAGCT | 7473 |
| Tcf3 | GCCCTTCCTGTGTGATGCTG | 7474 |
| Tcf3 | GTGCGTGTATACCGCCGCGT | 7475 |
| Tcf3 | GAGGCCGCGAGAAACTCAAC | 7476 |
| Tcf3 | GACAGTGGGCGTGGTCACTT | 7477 |
| Tcf3 | GGCGGGCAGACATAGAAGGA | 7478 |
| Tcf3 | GAAGGTGAGGGAGAGGGAGC | 7479 |
| Tcf3 | GATGAATTCCCACAGAAAGG | 7480 |
| Tcf7 | GTGAAACGGGCATCCCGGCT | 7481 |
| Tcf7 | GTTTCAACTGCTTTCCCAAG | 7482 |
| Tcf7 | GGTGAATGAGTCCGAAGGCG | 7483 |
| Tcf7 | GATCATCCCTGTGCCGATTA | 7484 |
| Tcf7 | GAGGTTGTCCCGGCTAACTT | 7485 |
| Tcf7 | GGCACAGCAGCTTTGGGAGC | 7486 |
| Tcf7 | GAAGAAGGCGCTAGAACCGG | 7487 |
| Tcf7 | GGTTGTCCCGGCTAACTTTG | 7488 |
| Tcf7 | GAGGTCGAGAGACCCGGAAT | 7489 |
| Tcf7 | GAAGCCTCTCAGCGTCTCAG | 7490 |
| Tcf7l2 | GGGCAGCCTGGGAGTTGAGA | 7491 |
| Tcf7l2 | GGCGGCCAACAATGATCCTT | 7492 |
| Tcf7l2 | GATGCTTTGGCCGCTAACTT | 7493 |
| Tcf7l2 | GAGGTGGTGGTGGACACCAG | 7494 |
| Tcf7l2 | GTGGTGGTATCCAGATGGGT | 7495 |
| Tcf7l2 | GGGTGGTCAGTGGGTGTTGA | 7496 |
| Tcf7l2 | GGGTTGATAATGGCATTAGA | 7497 |
| Tcf7l2 | GCACAATATAGACTATGCCA | 7498 |
| Tcf7l2 | GGGATAAGCATAAACAGTTG | 7499 |
| Tcf7l2 | GGCCATCTACAGGGAGGGTA | 7500 |
| Tead1 | GCCATAGGAAATGGGTCTTA | 7501 |
| Tead1 | GTGTTCTCTGAATGATGGCT | 7502 |
| Tead1 | GGCTCCTTTCTGGGAAACTA | 7503 |
| Tead1 | GTCTTATTCGCTGGTGTTAA | 7504 |
| Tead1 | GCTGGTCAGGCCATAGGAAA | 7505 |
| Tead1 | GATGTAGGCATTGATCTTTG | 7506 |
| Tead1 | GGATGCAGTTGGTAGAGTGA | 7507 |
| Tead1 | GAAGGGTAACTGGCCTGTCA | 7508 |
| Tead1 | GGACAGACATGCTGGCAGTT | 7509 |
| Tead1 | GTGAAACCATAGACCAAGCC | 7510 |
| Tead2 | GAGACCCAGAGAAAGTTGCC | 7511 |
| Tead2 | GCAAACACCCAGGGTGACCC | 7512 |
| Tead2 | CCTTAGACTTGGGATTTCTT | 7513 |
| Tead2 | GCAAGCAGGGACTGAGTGAG | 7514 |
| Tead2 | GGGCAGAGAGTTGGAACCCA | 7515 |
| Tead2 | GCCAGGCTCTGCCTCTAAGT | 7516 |
| Tead2 | GTCCATCTAAGGACTGGGTA | 7517 |
| Tead2 | GATGAGTCCATCTAAGGACT | 7518 |
| Tead2 | GTGGATCTTCAGAAACGCAG | 7519 |
| Tef | GTCAGGTTGCCCACTGATTT | 7520 |
| Tef | GGTCCCTAGGATGGTCGTTA | 7521 |
| Tef | GGCTGGAATCAAGAGGGAGC | 7522 |
| Tef | GGTTATGGTTCCCAGACTGG | 7523 |
| Tef | GTTACATTTGTGTGTGCAAG | 7524 |
| Tef | GAGCAACTGGATAATTCCCA | 7525 |
| Tef | GAATTATCCAGTTGCTCCAC | 7526 |
| Tef | GACTGGCCTGTTGTGTTGTT | 7527 |
| Tef | GCTCCACTCTTGGAAGGCTG | 7528 |
| Tef | GCGGAGCGATACTCCTCACC | 7529 |
| Tfam | GGGATGGATGGATGATGATG | 7530 |
| Tfam | GTACAGGTAGGCAGCAGAAG | 7531 |
| Tfam | GCTTCGTGACGCTGGTGCTG | 7532 |
| Tfam | GCCCAGGAGAACACATGGCA | 7533 |
| Tfam | GTCCCACAATTTCAGTGGTT | 7534 |
| Tfam | GATGGTAAACTGGGCTTAGA | 7535 |
| Tfam | GCAGCATTCCTCCTCAGCAG | 7536 |
| Tfam | GAAACATGCAGTTTGCTGTT | 7537 |
| Tfam | GATCTCTAACTTCAGTAGCC | 7538 |
| Tfam | GTTGTGACAGGAGGTTTGAA | 7539 |
| Tfap2a | GTCAGCTCTGGTCTTGTCTC | 7540 |
| Tfap2a | GTCGTTGATCCACAATTAGC | 7541 |
| Tfap2a | GGACGAAAGGCAGATAGTGG | 7542 |
| Tfap2a | GAGTTGTGGTAATGAAGCTC | 7543 |
| Tfap2a | GCGGTTTGGCTCACTCCAGA | 7544 |
| Tfap2a | GCAGTGTGGGCGCTGATGAA | 7545 |
| Tfap2a | GGACCCGAAGACAGGCGAAG | 7546 |
| Tfap2a | GTTGTTGTCCACGTTGCACC | 7547 |
| Tfap2a | GAGCGGAGGCGATCTCTAGT | 7548 |
| Tfap2a | GATTTGGCTGAGACTCTGCA | 7549 |
| Tfap2b | GAGGCTCCGTGAGTAGGAGA | 7550 |
| Tfap2b | GAGTCCTCAGCTTGGCTGTT | 7551 |
| Tfap2b | GCCGGAGCTCTAGAATGCAC | 7552 |
| Tfap2b | GTTCAGATTTCTTTCAGAAC | 7553 |
| Tfap2b | GCCAGTGCTATGTTTACTAT | 7554 |
| Tfap2b | GTGCCTCCCTACTGACTCCA | 7555 |
| Tfap2b | GGAGACTTTGCTCTCCAGAA | 7556 |
| Tfap2b | GTGCATTCTAGAGCTCCGGC | 7557 |
| Tfab2b | GAGGTGAGTGCATTCATGTG | 7558 |
| Tfap2b | GATTTCTGATCGGGTTTCTG | 7559 |
| Tfap2c | GGTATCAAGAATTGTGTAAT | 7560 |
| Tfap2c | GTCTCACATTTGGGCATTTA | 7561 |
| Tfap2c | GTGGTGGCAGGATAGGAGAC | 7562 |
| Tfap2c | GGGTAGCAGGGACACAGGAG | 7563 |
| Tfap2c | GTGAGAACCTTGACCATGGC | 7564 |
| Tfap2c | GGAAGTGACACTCTCAGGTA | 7565 |
| Tfap2c | GAGATATGGGAGTGGAAGGA | 7566 |
| Tfap2c | GAAGAGGATTGCGAGGTGAG | 7567 |
| Tfap2c | GCTCCGGTGCAGAAAGCACT | 7568 |
| Tfap2c | GCTTCAAACAGTGGAATTGG | 7569 |
| Tfap2d | GAGGGTGCTGGAAAGGGACA | 7570 |
| Tfap2d | GTGGGAAGATGATATAAAGA | 7571 |
| Tfap2d | GTACTTGCTGAGAATTTCTT | 7572 |
| Tfap2d | GGAGCATCTGTCTTCAGTTA | 7573 |
| Tfap2d | GTTAGTTGCTGAACACTCTT | 7574 |
| Tfap2d | GTCTGTAAGCTGCATGTATT | 7575 |
| Tfap2d | GGCCTGTCACTCAGAGCTAT | 7576 |
| Tfap2d | GCAATCAGTCTTGCCCAGTT | 7577 |
| Tfap2d | GGACTTGGTCTTTAAGTAGG | 7578 |
| Tfap2e | GAGGGAAGCAGGCACAGACA | 7579 |
| Tfap2e | GACTGGGAAGGACACATTTG | 7580 |
| Tfap2e | GGCTAGAACGAAGCCGAGGC | 7581 |
| Tfap2e | GCTGTGAAGCCTTTCTCTTC | 7582 |
| Tfap2e | GGAACCAGCAGCTATGATGG | 7583 |
| Tfap2e | GTTAGGAGACTCCATATTAA | 7584 |
| Tfap2e | GCGGGCAGAGTAAGAGGAGC | 7585 |
| Tfap2e | GACTCCTACTCTCTGTGCCT | 7586 |
| Tfap2e | GACAAATGGTCATTAGACTT | 7587 |
| Ifap4 | GAAAGAGGCAAGACAGGACC | 7588 |
| Tfap4 | GGCAGCCACCTGGGTAAAGA | 7589 |
| Tfap4 | GCAAGGTACCTCGGATGTTT | 7590 |
| Tfap4 | GCAAGTGGGAAGGAAGGGAA | 7591 |
| Tfap4 | GGAAGAGAGAGCAAGTGGGA | 7592 |
| Tfap4 | GACCCATTCGCTGCCTTCCA | 7593 |
| Tfap4 | GATTTGGGTGGAACCTTGGA | 7594 |
| Tfap4 | GAGAGAGCAAGTGGGAAGGA | 7595 |
| Tfap4 | GCGCTAGAATTCTGGATTAC | 7596 |
| Tfcp2 | GTAATTAATCCTCAGGAGCA | 7597 |
| Tfcp2 | GAGTTTAGGACTGGAGACAC | 7598 |
| Tfcp2 | GTCTCTTCTTCATTGGTAGA | 7599 |
| Tfcp2 | GCCGTAGCGGACGTCCTGAT | 7600 |
| Tfcp2 | GTCCTGATTGGTTGGCGCTG | 7601 |
| Tfcp2 | GAAGATAAGGGAAGGCAAAT | 7602 |
| Tfcp2 | GTTAGAGTCCTTTCAGTGAG | 7603 |
| Tfcp2 | GTGATAGGCTATCGACGCGA | 7604 |
| Tfcp2l1 | GCAGAGCCAGCAAAGGAAGG | 7605 |
| Tfcp2l1 | GGGAGAGCTGGGAAGGGAAG | 7606 |
| Tfcp2l1 | GTCAGGTGGGCGTGGCATTA | 7607 |
| Tfcp2l1 | GAGTTGGGTGTGATAGCTAC | 7608 |
| Tfcp2l1 | GTTCCTAGAGGTGAACCCAC | 7609 |
| Tfcp2l1 | GTCAGCTGTGACCTGAGTGG | 7610 |
| Tfcp2l1 | GTGGATGTGTGACAATGCAA | 7611 |
| Tfcp2l1 | GTGCCTATGCTTGCAGGCCC | 7612 |
| Tfcp2l1 | GCAAGTGCCAGCTCCCAGAA | 7613 |
| Tfcp2l1 | GGAGATGGGATGAAGTGTCC | 7614 |
| Tfdp1 | GATGCTAAAGAAGGGCTGTT | 7615 |
| Tfdp1 | GCATATCTGTGTGCATGCGC | 7616 |
| Tfdp1 | GTCAGTAGTTGAGCAGAGGT | 7617 |
| Tfdp1 | GACAAGACTTGCCACCTCCC | 7618 |
| Tfdp1 | GTCATGGGACTGAGGCAGCC | 7619 |
| Tfdp1 | GTGCATGCGCAGGGAGTAGA | 7620 |
| Tfdp1 | GCTATTGACACACTCATGCC | 7621 |
| Tfdp1 | GCTTCAGCAATAAGCAGCTG | 7622 |
| Tfdp1 | GGGAGCATTTCCATTTAGAG | 7623 |
| Tfdp1 | GCCTGGGCTGTCAGTGATTC | 7624 |
| Tfdp2 | GTTTCATGGATCCTTTGGTT | 7625 |
| Tfdp2 | GACAGACACCTTAGTTATGT | 7626 |
| Tfdp2 | GGCTGGCACATTAAATGTTT | 7627 |
| Tfdp2 | GCAAACACTGGTTTCACAGC | 7628 |
| Tfdp2 | GCTAAACTGAAACTATGAGT | 7629 |
| Tfdp2 | GAGCTTTGCTGTATGGAACC | 7630 |
| Tfdp2 | GAATAAATTGACTTGCTCCA | 7631 |
| Tfdp2 | GAAGCATGGCCTATCTCAGG | 7632 |
| Tfdp2 | GTTTGGGTTAATGTGGAGAA | 7633 |
| Tfdp2 | GCCAACAATGATATGATACA | 7634 |
| Tfe3 | GAACTGAGAGACCGGCTGGG | 7635 |
| Tfe3 | GTCTCAGGATGCTGGGAAGT | 7636 |
| Tfe3 | GGGAGGCAGAGGTGTTATTT | 7637 |
| Tfe3 | GAGGAGGCAGCGGCAAACAG | 7638 |
| Tfe3 | GAGGAGGGCAGAGTCATATT | 7639 |
| Tfe3 | GCTCCATGGCTTAACGGAGG | 7640 |
| Tfe3 | GCTGCCTTGCTCAGGAGGTA | 7641 |
| Tfe3 | GAGTCATCACCCGCCCTGAA | 7642 |
| Tfe3 | GCGGTAGGGAACACCGGCTT | 7643 |
| Tfe3 | GTAGTGGGAGTGCGTGAGGA | 7644 |
| Tfeb | GAGCTTCCAGCAGGAGGGAC | 7645 |
| Tfeb | GTTGTCATTGCCTGGGTTGT | 7646 |
| Tfeb | GTCAGATTCCTTGGGTCTCG | 7647 |
| Tfeb | GTTGAGTCATTTGTATGTAG | 7648 |
| Tfeb | GTGAAATGGAGTTGGAAGCC | 7649 |
| Tfeb | GACATGGGCAATAACAGGGT | 7650 |
| Tfeb | GGTATGAAATACTCAGGATG | 7651 |
| Tfeb | GTGGAAGTTGCTAAGGGATA | 7652 |
| Tfeb | GATGACACTGAGTAAGTCCC | 7653 |
| Tfeb | GGAGTCTTGATTGCAGATAT | 7654 |
| Tfec | GAATGGAGACAAACAGCTCG | 7655 |
| Tfec | GTGTAATTCCTAACTGAAAG | 7656 |
| Tfec | GAATTTACAATGGCAGTATG | 7657 |
| Tfec | GTGCTGTGGTCATGCATTTG | 7658 |
| Tfec | GTCCTTGCCCACTTTCAGTT | 7659 |
| Tfec | GCCATTACTGCAGCAGAACT | 7660 |
| Tfec | GCAATAACGGCATCAATGAG | 7661 |
| Tfec | GAATGTGTTGCAATAACTGT | 7662 |
| Tgfb1 | GGCGGCAGGGACAGAATGTA | 7663 |
| Tgfb1 | GGCACAGTGCACCTTGGTAT | 7664 |
| Tgfb1 | GGTTTCAATGCTGGGAACCC | 7665 |
| Tgfb1 | GCAGCAGCAGGCCGATACCA | 7666 |
| Tgfb1 | GGCCACTAGAAACCTAACGA | 7667 |
| Tgfb1 | GGGTAGAAAGGGCTGTGGGT | 7668 |
| Tgfb1 | GTTGGAGGGAGCAGCTAGCA | 7669 |
| Tgfb1 | GATCGAAGTGGCGCAGCAGC | 7670 |
| Tgfb1 | GTGGGTGAGAAGGACAGTGG | 7671 |
| Tgif1 | GAACAACTATTCCTTGTATG | 7672 |
| Tgif1 | GAGCAGAAGGTGTGCACTGG | 7673 |
| Tgif1 | GAGTTCCGGATTGGATTGCA | 7674 |
| Tgif1 | GCTGTGTCTCTGATGGCAGT | 7675 |
| Tgif1 | GCTCTTTAAACGTCTGGGCT | 7676 |
| Tgif1 | GCACCAGTGCGGGACATGTG | 7677 |
| Tgif1 | GGTGCGGTTCATATCCTCAA | 7678 |
| Tgif1 | GTGTGGGCGCTCTGAGTTGG | 7679 |
| Tgif1 | GAAGGAATCCTTCAATTGCA | 7680 |
| Tgif1 | GATCAAAGGGCAGGCTTTAG | 7681 |
| Tgif2 | GCCACCTCCAAATTGCGACA | 7682 |
| Tgif2 | GAAGACTGTTCGGATGCTGT | 7683 |
| Igif2 | GAGGCGAACTTACAAAGGTA | 7684 |
| Tgif2 | GCCTGGATCGTATGAGATCC | 7685 |
| Tgif2 | GCGGCATTCCTAAGGTGGGT | 7686 |
| Tgif2 | GCCCTCTCCCAGAGGAGCTT | 7687 |
| Tgif2 | GCCAGTGTCCTTTGGCCAGG | 7688 |
| Tgif2 | GACCCGCTTGGCAGGATCCA | 7689 |
| Tgif2 | GGCATCTGTCACTCCCAGGC | 7690 |
| Tgif2 | GGCACATCTCCAGATTCACT | 7691 |
| Thra | GGAGCCAGAGAGTGTGTCTG | 7692 |
| Thra | GTACAGCGGCAGCTGCAGTG | 7693 |
| Thra | GTCTGTTATCAGCCCATATT | 7694 |
| Thra | GCTGGAACTAAGATGTGCAA | 7695 |
| Thra | GTTGCATGTCCGCTGGGAGC | 7696 |
| Thra | GATAGAATCGTTTGCTGAGT | 7697 |
| Thra | CATCCTAGCATCTGGCGAGA | 7698 |
| Thra | GCAAACGTCTCTGTTCTCCA | 7699 |
| Thra | GAAGTAGGCTCTTGGGATTG | 7700 |
| Thra | GTGACTAGAAAGAGCTTCCA | 7701 |
| Thrb | GAGTTACAACCCAAGCATGG | 7702 |
| Thrb | GCTGTAAAGGTCTTAAGCTG | 7703 |
| Thrb | GGGTGAGGGAGGAACACATG | 7704 |
| Thrb | GTAATCATTFCCTAATCACG | 7705 |
| Thrb | GCTCCAACAGGAAATTTGGT | 7706 |
| Thrb | GACCTTCGGAACTTGAGGTA | 7707 |
| Thrb | GCAAGTGCAGAACCCTTCCA | 7708 |
| Thrb | GGGCTGTCTGGTTAGGAACT | 7709 |
| Thrb | GACTCTCAGGTGGGAGTCAC | 7710 |
| Thrb | GGAGGTGGCAGATTCAGACA | 7711 |
| Tle4 | GAGCCTGAGGGCTATTGAAA | 7712 |
| Tle4 | GTTTGTGTGTTCACAAGCCC | 7713 |
| Tle4 | GGGTTCTGACCCTCTTCCGT | 7714 |
| Tle4 | GCTGTCAATCAAAGTAACGT | 7715 |
| Tle4 | GCATCTTTAGAAGCAGGTTT | 7716 |
| Tle4 | GCCCAATTCAAGGCGTTCTG | 7717 |
| Tle4 | GCCTGCACTTCGAGTTAAGG | 7718 |
| Tle4 | GGCACCAGTCAACTTACTCC | 7719 |
| Tle4 | GATGACTTTGGTGGCACTAA | 7720 |
| Tle4 | GCACTAGGGAACAGCGGCCA | 7721 |
| Tlx1 | GGTATGCGGAGTAAATGCCC | 7722 |
| Tlx1 | GCTAACCACGCAATCTCAGT | 7723 |
| Tlx1 | GGTGCTTGTCCCAGGGTAGC | 7724 |
| tlx1 | GCCTTACAGAGTAGCCCTGT | 7725 |
| Tlx1 | GAAAGAGGTACCTTGAGGAA | 7726 |
| Tlx1 | GAAGTACAGCACTGGTGGGA | 7727 |
| Tlx1 | GAACCTTTCCCAGACCTGGT | 7728 |
| Tlx1 | GACAGACGGACCAAAGGAGA | 7729 |
| Tlx1 | GTGCTTGTCCCAGGGTAGCT | 7730 |
| Tlx1 | GACCAGCAGGTGTCAGGAGC | 7731 |
| Tlx2 | GTAAGCACAGCCGCCCTTTG | 7732 |
| Tlx2 | GCCAAAGTGGAGCACTGGAT | 7733 |
| Tlx2 | GCTAGTTCAGGTTGAAGATG | 7734 |
| Tlx2 | GGTCCAGGCCTGTCATAGTG | 7735 |
| Tlx2 | GGGAGTGGAGGTGCAGATAG | 7736 |
| Tlx2 | GTGTTAACCCAGTGGAGGGT | 7737 |
| Tlx2 | GAAGTCAGGCAACTCAGGGT | 7738 |
| Tlx2 | GTCACAGGGTGGGAGGTAGA | 7739 |
| Tlx2 | GGATATTTGGGCATCTGGAC | 7740 |
| Tlx2 | GGTGTTAACCCAGTGGAGGG | 7741 |
| Tlx3 | GGAATTGATGGTCAGACTGG | 7742 |
| Tlx3 | GGTCACCTTTCTCTCGGTCT | 7743 |
| Tlx3 | GGTATGAGATGACCAGGACA | 7744 |
| Tlx3 | GTGTCCAGGCCTGGAACCCA | 7745 |
| Tlx3 | GAACGGTCCTACGAAATCTG | 7746 |
| Tlx3 | GACCCAGAGTCATTTCTTAG | 7747 |
| Tlx3 | GCAGAGATGCAACCCAAGAA | 7748 |
| Tlx3 | GGACCCGAGGTCAACTGGTG | 7749 |
| Tlx3 | GGTTTGAGAAGCTGCGGTTC | 7750 |
| Tlx3 | GGAGCTTAGGGACTGTTCCA | 7751 |
| Trerf1 | GACTTAGGAGAGTCGAGCTG | 7752 |
| Trerf1 | GCAGGAAGCAATCCTGCAAT | 7753 |
| Trerf1 | GACAGGGCTATGACAGAAGA | 7754 |
| Trerf1 | GAACCCTCAGATCCCTTCCT | 7755 |
| Trerf1 | GGCGATAAGACAGGTACAAG | 7756 |
| Trerf1 | GTTCCCTGAGCGAGTTGGCT | 7757 |
| Trerf1 | GCGTTGCAAATCTAACGACT | 7758 |
| Trerf1 | GTGGGATGGGTCAGGGACAG | 7759 |
| Trerf1 | GGCTCTGACTGAAGGAAGAG | 7760 |
| Trerf1 | GTCTCTGAACTGGAGAGGAT | 7761 |
| Trim24 | GGAGCTTTAGAGAAAGAGTA | 7762 |
| Trim24 | GCTGGATTAAGGTTTACAGA | 7763 |
| Trim24 | GAACTACTGCACIATGGGCC | 7764 |
| Trim24 | GATTTACAGCTTGCCTGCAT | 7765 |
| Trim24 | GGAGTCATTTGAAGCCACAC | 7766 |
| Trim24 | GAATTTCCGGTAACAAGCAC | 7767 |
| Trim24 | GGTGTGTCCAGTGAGGTCAA | 7768 |
| Trim24 | GTAACAGGTGGCACTTCCCA | 7769 |
| Trim24 | GGGCAGACTCAGCTGGATTA | 7770 |
| Trim28 | GCCGAGGAAGGAACAAAGGC | 7771 |
| Trim28 | GTTCAGCGCTCACCCTTCGG | 7772 |
| Trim28 | GAACCAGGTGTTCACCCACG | 7773 |
| Trim28 | GGAAGTGAGAGTCCAGAGGC | 7774 |
| Trim28 | GGTAGGAAGTGAGAGTCCAG | 7775 |
| Trim28 | GGAAGCTGGCAAAGCAAGCA | 7776 |
| Trim28 | GGGACAATACAGGGTGGGCG | 7777 |
| Trim28 | GTTGCCCGCAAGCAGTTCCA | 7778 |
| Trim28 | GGTTCACAGGCACCCTATCC | 7779 |
| Trp53 | GATGGCTATGACTATCTAGC | 7780 |
| Trp53 | GGAGGATGCGGAGAGCCTAT | 7781 |
| Trp53 | GGTTGGTCATCACCACCGCA | 7782 |
| Trp53 | GGCTAGGTTGGTTGTGTCCC | 7783 |
| Trp53 | GAACGCGCTGAAGTGGATGA | 7784 |
| Trp53 | GTCTGTCAACAGGTGACGCA | 7785 |
| Trp53 | GTAAGTGACACTGGAATCTG | 7786 |
| Trp53 | GGATAGCCTCCCTCCTGACC | 7787 |
| Trp53 | GCCTAACCCAGGACTATACA | 7788 |
| Trp63 | GCTGAAAGGGAGGCAGAAGG | 7789 |
| Trp63 | GACACTAGAAGCCAAGACTT | 7790 |
| Trp63 | GGAAGTCTGTGTCTTTGCCT | 7791 |
| Trp63 | GTCAGATTTGGCTGGAGCGC | 7792 |
| Trp63 | GATGCATGTTGCAGATTTCA | 7793 |
| Trp63 | GTCTATGGCTGTGGCATGAA | 7794 |
| Trp63 | GAGGACCACTACCAGGTGCA | 7795 |
| Trp63 | GAAGCTGAGTTCAGGGCAAC | 7796 |
| Trp63 | GATAGTACGACCCTCTTCCA | 7797 |
| Trp63 | GATCCCATGCCAGGGATCCC | 7798 |
| Trp73 | GGGCAGTGGTATCCACTGTA | 7799 |
| Trp73 | GTCTTGGGAGATTGAGTGGA | 7800 |
| Trp73 | GAGGGAGAAGGAAGCTGGTT | 7801 |
| Trp73 | GATCCCTACGCTGGTCTGAG | 7802 |
| Trp73 | GAAACCACTGGAAGTGGTGG | 7803 |
| Trp73 | GAGTCAGGTGTGAGTTCTCA | 7804 |
| Trp73 | GTGTTGTGGAGAGAACGCCA | 7805 |
| Trp73 | GTGGACTTGATTCAGAGGTG | 7806 |
| Trp73 | GGCTCACAGACTCTCAGGCC | 7807 |
| Trp73 | GCAAGCTCTCTGGAGGGAGA | 7808 |
| Tsn | GACACTTGGCTCGTAGAAGG | 7809 |
| Tsn | GAGCATGCATAAACTGAATG | 7810 |
| Tsn | GAGTATCATGAAGATCTCTC | 7811 |
| Tsn | GTCTACCACATCCTGAACTT | 7812 |
| Tsn | GGCTGGTGAGAAGTGTTGGG | 7813 |
| Tsn | GTTACTGTAACCTTCAACCA | 7814 |
| Tsn | GCAGCAACATTTGGGAAGAG | 7815 |
| Tsn | GTGGCTCCAGCAGCAACATT | 7816 |
| Twist1 | GAAAGTACAGTCGGGTTTAC | 7817 |
| Twist1 | GCAGAAAGCGGTGTCTTACC | 7818 |
| Twist1 | GAGAGCCCAGACGTTTCTCC | 7819 |
| Twist1 | GGTGTCATTGGCCTGACGTG | 7820 |
| Twist1 | GCGGTGTCATTGGCCTGACG | 7821 |
| Twist1 | GTCGGAAACCTCTAGTCCCA | 7822 |
| Twist1 | GGAGAACTCCGAGGGATCCC | 7823 |
| Twist1 | GGCGAATCAACTCTCAGCAA | 7824 |
| Twist1 | GGTGAGGAGAAATAGTACCC | 7825 |
| Twist1 | GCAGCCCATCTCAGCTTGTC | 7826 |
| Twist2 | GAACCTATTCCCAGGTGACC | 7827 |
| Twist2 | GCTCAGTTCAGCCAGAGGAT | 7828 |
| Twist2 | GTGGCTCCTTAAACATTCTC | 7829 |
| Twist2 | GGCGTCTCTATAGATCCTAG | 7830 |
| Twist2 | GCCTGGGCTCTCCACCTTTG | 7831 |
| Twist2 | GGCAGGGCCAAATCTGCTCC | 7832 |
| Twist2 | GGAGCAGATTTGGCCCTGCC | 7833 |
| Twist2 | GAGCAGATTTGGCCCTGCCA | 7834 |
| Ubp1 | GGTATCTGTGTGTGTGTGTG | 7835 |
| Ubp1 | GTTCTTCTCAAAGCTTGTTA | 7836 |
| Ubp1 | GGGAAGGAAGGCAGCCAAAT | 7837 |
| Ubp1 | GGACGATCCATGCCTTGGGA | 7838 |
| Ubp1 | GGACCCACATCCGAATCCTT | 7839 |
| Ubp1 | GATCCATGCCTTGGGAAGGA | 7840 |
| Ubp1 | GCAATAATCGAGGGCCGGCT | 7841 |
| Ubp1 | GGCCAATGAGCTCTTACTTA | 7842 |
| Ubp1 | GTTCCAGGTCCTACTTCCGT | 7843 |
| Ubtf | GGAGCAGATGAGGACTAGGC | 7844 |
| Ubtf | GGGTTTCTTCCGTGCGTGTT | 7845 |
| Ubtf | GAAGGGCTGCAACCAAGTGC | 7846 |
| Ubtf | GGTCATGAGTCTTAAGATGT | 7847 |
| Ubtf | GGTTTCTTCCGTGCGTGTTG | 7848 |
| Ubtf | GTTACTGAAGCCCAGGGTAA | 7849 |
| Ubtf | GGAGCAATGGGAGAGGGAGC | 7850 |
| Ubtf | GAAACGCAGAGCGATGGAGG | 7851 |
| Ubtf | GCGTTTCTCTTCCAATCAGC | 7852 |
| Ubtf | GACACACACCAGTGGCGACA | 7853 |
| Uncx | GATGATCAGGCTTCCTTCTG | 7854 |
| Uncx | GATGCCACCCCAGAGGAGGA | 7855 |
| Uncx | GATGATGAATCTCCCATTAT | 7856 |
| Uncx | GGCCCOGACATGAGTGTTGG | 7857 |
| Uncx | GCAAACTTGTCACTGTGCCC | 7858 |
| Uncx | GCTCCTTCAGAGACAGAGGG | 7859 |
| Uncx | GCATCCAGGACCCATATGTG | 7860 |
| Uncx | GCTGTGATCAATCCAGCCCG | 7861 |
| Uncx | GGGTTAGACTCCTTATAGGT | 7862 |
| Usf1 | GGGCCACAAGAGGGACAACA | 7863 |
| Usf1 | GGATCAGGGCATCACTTTGA | 7864 |
| Usf1 | GGCCACCATTTAGCAATGGT | 7865 |
| Usf1 | GGATGGAAGTACAATTTAGT | 7866 |
| Usf1 | GCTACAGCCATCTGAACCAG | 7867 |
| Usf1 | GCAAGCCCATGTCCAAGGCC | 7868 |
| Usf1 | GGATCCAGAGCATGTGTTCC | 7869 |
| Usf1 | GACCTGTATTCTTGTCCCTG | 7870 |
| Usf1 | GAGGGTGATGATAGGAAGGA | 7871 |
| Usf1 | GACCTGTCGGACCTGAACTA | 7872 |
| Usf2 | GGCCACCAACTAATAGAGAC | 7873 |
| Usf2 | GTCTGTCTTTGGTGACGGCC | 7874 |
| Usf2 | GGTCCTATACTATATGGAGA | 7875 |
| Usf2 | GAGTCCCAGAACATGGGAGC | 7876 |
| Usf2 | GGGAGGACGCATGGGAATCA | 7877 |
| Usf2 | GGAATCATGGCAGGCGGAGG | 7878 |
| Usf2 | GAGGCCCAATCCATACATGG | 7879 |
| Usf2 | GTATAGGAGCCCGGAGGTTG | 7880 |
| Usf2 | GCCCTCTCCGTCCACTACTT | 7881 |
| Usf2 | GCCAGCAGCATAAACTGGGA | 7882 |
| Utf1 | GTTGCCTAAAGTGTCCGAAC | 7883 |
| Utf1 | GAACCTCACCTAGGATCTCC | 7884 |
| Utf1 | GAGGAACTAGGTAGGCGAGG | 7885 |
| Utf1 | GAACCTTACATCTCAGGTCC | 7886 |
| Utf1 | GTGATGGGATCTGGTGGCTC | 7887 |
| Utf1 | GGACAGATGCATTAGAGGTG | 7888 |
| Utf1 | GGGCTTTGGCTCACTGGGAA | 7889 |
| Utf1 | GCCAATCAGTAGAAACTGGT | 7890 |
| Utf1 | GTAAGCGGGACTGAGAGCCC | 7891 |
| Vav1 | GGGCACAAGTGCAAAGGCCC | 7892 |
| Vav1 | GAATTGTCTTGGTTTACCGT | 7893 |
| Vav1 | GCAATACTACGTTTATTCAA | 7894 |
| Vav1 | GCAGTTAGGGTAGGAAGGCC | 7895 |
| Vav1 | GCGGCGCTAAACGGCTTCAC | 7896 |
| Vav1 | GGCACAAGTGCAAAGGCCCT | 7897 |
| Vav1 | GGCCTCTAGGCGGCGCTAAA | 7898 |
| Vav1 | GACAGTTACAGTCACAGAAG | 7899 |
| Vav1 | GTTAGAGGAAGTCGAGGGTT | 7900 |
| Vax1 | GGATTCCTGAGGCTTCGGAT | 7901 |
| Vax1 | GTTGAGTGATGTTCACTGAG | 7902 |
| Vax1 | GGAGTTGACTTTATATGATT | 7903 |
| Vax1 | GTCCTCTGGGAAACCTGTCA | 7904 |
| Vax1 | GGTGTGTTAAGGAGTCGCTT | 7905 |
| Vax1 | GCTACCTGATCGCCAGGCTG | 7906 |
| Vax1 | GAACTAAGTCAGAGCCGACC | 7907 |
| Vax1 | GGAGGTGACAGCCGGACTGT | 7908 |
| Vax1 | GCTCACATACTGGCTAAAGG | 7909 |
| Vax1 | GTTGTGGTTGTCCGACACCC | 7910 |
| Vax2 | GCTTCTGTTTAGACAGTGTC | 7911 |
| Vax2 | GAGTGACAACCTCAGAGCTG | 7912 |
| Vax2 | GCCAGTGAGTGCCACAGTCA | 7913 |
| Vax2 | GACACCCGCTACCAGATCTT | 7914 |
| Vax2 | GTGCCTGAGTGTGAGTGCCT | 7915 |
| Vax2 | GGTGTTTAGAGCCTGGCAAT | 7916 |
| Vax2 | GCTTGCTGCTTCCCTCTTGC | 7917 |
| Vax2 | GATGGATGAGGTTGGAAGAG | 7918 |
| Vax2 | GAGAAATTACAACACAAAGG | 7919 |
| Vdr | GTTCTCAACAGCCAACACTT | 7920 |
| Vdr | GACGGTAGTGGAAACATTCT | 7921 |
| Vdr | GAAGCTACAGCAAGGCTTGC | 7922 |
| Vdr | GAGGCAGTGTGAAATGATGG | 7923 |
| Vdr | GGTGAGAAACCCTGGGCTAG | 7924 |
| Vdr | GTCCCGGGTCAACTCAGGTA | 7925 |
| Vdr | GTTAGAAGGTGAGAAACCCT | 7926 |
| Vdr | GACCTCACACATACCTGGGT | 7927 |
| Vdr | GTATATGTTCCTCTAGCCCA | 7928 |
| Vdr | GTTGCCGGGAGATGGTGGAA | 7929 |
| Vezf1 | GTGGTCTTCCAATTTGAGCA | 7930 |
| Vezf1 | GGACTTGTCCTCATCACCCA | 7931 |
| Vezf1 | GAAGGGAATCTTCTAAGGCA | 7932 |
| Vezf1 | GGAAGCTGACTTGTTTGGGA | 7933 |
| Vezf1 | GATGGCAAGAGCGCTGAGTG | 7934 |
| Vezf1 | GAATAGAAACTGAAGAGGTA | 7935 |
| Vezf1 | GGAATATAATCAAACCAAGC | 7936 |
| Vezf1 | GTTATAACACTTCAGGTGGA | 7937 |
| Vezf1 | GCACACACAAGTCAGACTTC | 7938 |
| Vsx1 | GCCTTCCACAGAACCAGGCT | 7939 |
| Vsx1 | GCAAGCCAGTAACTTTCCTT | 7940 |
| Vsx1 | GCAAGGGAGATGCGCTGTGT | 7941 |
| Vsx1 | GTCTTTATCCACCCAGAGGT | 7942 |
| Vsx1 | GGCTATCTGTCCGCCTGATT | 7943 |
| Vsx1 | GGCTGCCAACACACCAGGGT | 7944 |
| Vsx1 | GGACAGCTGGAGAGAGAAAG | 7945 |
| Vsx1 | GGGACAGCTGGAGAGAGAAA | 7946 |
| Vsxl | GAACCGTCCCTAGATCTTAC | 7947 |
| Vsx2 | GTCGCAGCTAACCTAGGCAC | 7948 |
| Vsx2 | GAGCTGAACAGCCAATCACC | 7949 |
| Vsx2 | GCTTCTCCAGAGGCTCTAGA | 7950 |
| Vsx2 | GCAACAAGGAGCTAAACTGA | 7951 |
| VSx2 | GAACATAATGTCCCGTGCTG | 7952 |
| Vsx2 | GGTGGTGGAGTAAGGAGGAC | 7953 |
| Vsx2 | GGTTAGCTGCGACAGATCCC | 7954 |
| Vsx2 | GAGGCTGCTTAGTTAAGGGA | 7955 |
| Vsx2 | GGGTTAGGATCGAGCCCTCG | 7956 |
| Vsx2 | GCTAGTCACTTTGAGCAGAA | 7957 |
| Wt1 | GTTATCCTTTCTGAGGCCCG | 7958 |
| Wt1 | GAGAACTCTCCTGGGTTCTG | 7959 |
| Wt1 | GCGCCTTGTTGAGAAGAAAC | 7960 |
| Wt1 | GCTGTTTGGAATCTTGGAAC | 7961 |
| Wt1 | GACGCCTTGCTACACTGACT | 7962 |
| Wt1 | GGCTGTTAATCAGGAAGGGT | 7963 |
| Wt1 | GAGCAATTGCCGGTTCCTCT | 7964 |
| Wt1 | GTTTCATTACCAAAGGAAAG | 7965 |
| Wt1 | GCAAATAACTTTCTGAGCCT | 7966 |
| Wt1 | GCTGGTGGCAGTCAGGCATC | 7967 |
| Xbp1 | CCCATGTGCCAAGCACGGAG | 7968 |
| Xbp1 | GCCTTGGCTAGCATGTAGTA | 7969 |
| Xbp1 | GTCACGCAGGAGGCTAGAAC | 7970 |
| Xbp1 | GGACAAAGCCCAAGATGCAG | 7971 |
| Xbp1 | GCATGTGCCAAGCACGGAGT | 7972 |
| Xbp1 | GTGCTAGAGCATTAGGTTCT | 7973 |
| Xbp1 | GTATTCCTTTCATTAGGGAA | 7974 |
| Xbp1 | GGAGGAGAGCCAGGCTCATT | 7975 |
| Ybx1 | GGAATAAACGTTAACTGCTG | 7976 |
| Ybx1 | GGTGGTGACATTACAGGCAA | 7977 |
| Ybx1 | GCCTATTGGCTCACGCTCCG | 7978 |
| Ybx1 | GGCTGCAGAGAGAGAAGGGA | 7979 |
| Ybx1 | GGCCTGAGAAGCTGTGGGTC | 7980 |
| Ybx1 | GGTGATCCAGTCACCTTGGA | 7981 |
| Ybx1 | GAGAGAAGGGATGGGAGTGG | 7982 |
| Ybx1 | GTGCTCACCCAACCAAGAAG | 7983 |
| Ybx1 | GGCGAATCTCCTCACAATTC | 7984 |
| Yy1 | GGAGTTGTTAGTGTTGAGGC | 7985 |
| Yy1 | GCCTGTTGGAACGAAGGGTC | 7986 |
| Yy1 | GCTTCATCTGTTGAATATTC | 7987 |
| Yy1 | GCAATTTGATGATTTGAACA | 7988 |
| Yy1 | GTTCATACAGTGTCTTTCAA | 7989 |
| Yy1 | GTGTGCTGCCACGGGCTCTT | 7990 |
| Yy1 | GGACCCTGGTTGGGAGTAGG | 7991 |
| Yy1 | GCCAGACCCTTCGTTCCAAC | 7992 |
| Yy1 | GTATGAATGTGGGAAGGCTG | 7993 |
| Yy1 | GGAGTTGGTATTTGTGTGGA | 7994 |
| Zbtb12 | GGCAGCAGTGATCCTAAGAT | 7995 |
| Zbtb12 | GACAAATAATCCUGGCCAAA | 7996 |
| Zbtb12 | GACAAGCTGAGGGCAAGCGC | 7997 |
| Zbtb12 | GGACAAATAATCCTGGCCAA | 7998 |
| Zbtb12 | GCAGTGATCCTAAGATTGGT | 7999 |
| Zbtb12 | GGAAGAATGCATATTTCACT | 8000 |
| Zbtb12 | GGATTGAGAAGCTTCCTGGA | 8001 |
| Zbtb14 | GCAGCAGAGGAAGTGGTGAC | 8002 |
| Zbtb14 | GGTTGACTCTTGCTATCAGC | 8003 |
| Zbtb14 | GGATGCCTAAGTAAACATGA | 8004 |
| Zbtb14 | GGTTGCTTTCCCTGGGTCTA | 8005 |
| Zbtb14 | GAGAAGTGGAGGATGGTGGA | 8006 |
| Zbtb14 | GTGTGTGCTCGTGCAGGAGG | 8007 |
| Zbtb14 | GCAGTAGTTAATAGGGATCA | 8008 |
| Zbtb14 | GCCACTGAGGCATTGTTTAT | 8009 |
| Zbtb14 | GCTGGTCCTTGCTTATCATC | 8010 |
| Zbtb16 | GCGCTCTTGAGTACTGGAGT | 8011 |
| Zbtb16 | GAACTTTCCATCCAGGTTCC | 8012 |
| Zbtb16 | GGAATCAAGATACGATTCTG | 8013 |
| Zbtb16 | GTGGTCAGGCATCAGAGGCC | 8014 |
| Zbtb16 | GGGATACACCGCATGCCCAG | 8015 |
| Zbtb16 | GGCCAGCCTTCATCGCTAAG | 8016 |
| Zbtb16 | GGAGGAGACGGTGCTTTCCG | 8017 |
| Zbtb16 | GCTAGATGTCAGGAGAAGCC | 8018 |
| Zbtb16 | GAGTAATGCCTAGATTTAAG | 8019 |
| Zbtb16 | GCTTACGGAGCCTGGAGCTT | 8020 |
| Zbtb18 | GAGCAGGAAGACAAGCTGTG | 8021 |
| Zbtb18 | GCTCTGACATCATGTTCAGA | 8022 |
| Zbtb18 | GGGTGTGTGTGTGAGGTTCA | 8023 |
| Zbtb18 | GTCTGCCATTATCTCCGCAG | 8024 |
| Zbtb18 | GTAAACTGGGTGATTAATGT | 8052 |
| Zbtb18 | GCCCGAAGACATCCACTCCA | 8026 |
| Zbtb18 | GCAGACGGCGACTGTTGGGT | 8027 |
| Zbtb18 | GTTGTGATCACCAGGAGGGT | 8028 |
| Zbtb18 | GCCATATGGCCACAGTCAGC | 8029 |
| Zbtb20 | GGTCGTTGAACTGCTCGATT | 8030 |
| Zbtb20 | GCACCTAAGTGTGGCTCAGA | 8031 |
| Zbtb20 | GGGCGACTGAAGACCAAGTG | 8032 |
| Zbtb20 | GATGCCTGCTGGTCAGACCT | 8033 |
| Zbtb20 | GAGAGGGACAGAGGGAGGAC | 8034 |
| Zatb20 | GCCTGTTCCTCTATGAGGTA | 8035 |
| Zbtb20 | GACAGGCTGATCAGACTGCC | 8036 |
| Zbtb20 | GGCCTAGATCTTTCCAATCA | 8037 |
| Zbtb20 | GTTGCTTGTCCGAGTCTTCC | 8038 |
| Zbtb20 | GAGTGACAGAGACAGAGAAA | 8039 |
| Xbtb3 | GCTTGAGCCAATTAAGATAG | 8040 |
| Zbtb3 | GGTGGAGAATGACTTAGGGA | 8041 |
| Zbtb3 | GCAATTAGGGAACTGGTTGA | 8042 |
| Zbtb3 | GGCTACAGGAACATTATGCT | 8043 |
| Zbtb3 | GTCATGTGCAATTAGGGAAC | 8044 |
| Zbtb3 | GCCAATTAAGATAGCGGAGG | 8045 |
| Zbtb3 | GTCAGCAAGCACAGCTGAGG | 8046 |
| Zbtb3 | GGGAAATTACCCGCCTGGGT | 8047 |
| Zbtb32 | GATCAGACAGGGTGTGTCGG | 8048 |
| Zbtb32 | GGAAGGcATCCTAGGTCTGG | 8049 |
| Zbtb32 | GTTGTAGCGGGAAAGGCACT | 8050 |
| Zbtb32 | GTGGGTGAAGCATACTAGTG | 8051 |
| Zbtb32 | GAAGCTAGGGAGAAACCTCA | 8052 |
| Zbtb32 | GGAGAGCATTATGAGAGGTG | 8053 |
| Zbtb32 | GAGGGATAAAGGCAACTACA | 8054 |
| Zbtb32 | GCACCCAAGCTAGAATCGGA | 8055 |
| Zbtb32 | GAAGAGTAATCACAGACACT | 8056 |
| Zbtb32 | GCGACCCTCCTAGATCAGAC | 8057 |
| Zbtb33 | GGAAGCCGCTTTGACGTCGG | 8058 |
| Zbtb33 | GCCACTCCTAACAGTGTCAT | 8059 |
| Zbtb33 | GCAGTCACGGAAAGAGCCGA | 8060 |
| Zbtb33 | GTCACGGACAGAGCCGAAGG | 8061 |
| Zbtb33 | GCTTTGACGTCGGCGGAAAC | 8062 |
| Zbtb5 | GCCTGTGGAGGCGGTGACAT | 8063 |
| Zbtb5 | GATCTAGGTATAGTGGTGTA | 8064 |
| Zbtb5 | GTTGTTCTCTGTTGTGAGTG | 8065 |
| Zbtb5 | GTCTCCGCTTTGATGTTTAT | 8066 |
| Zbtb5 | GATTGGCCATTGGAGGCCTG | 8067 |
| Zbtb5 | GTGATTGGTCAGAGCGCTCA | 8068 |
| Zbtb5 | GTTCTAGTTCTGAGTTAACC | 8069 |
| Zbtb5 | GAGTTGTTGAAGCTGGTGAA | 8070 |
| Zbtb5 | GTTAATCTGGAAAGGAAACT | 8071 |
| Zbtb5 | GAGAGAATGTCAGGAGCTAA | 8072 |
| Zbtb7a | GTAATTTAAGGCGCAGATGG | 8073 |
| Zbtb7a | GGTAATTTAAGGCGCAGATG | 8074 |
| Zbtb7a | GAGTAAACTGAGGTTTATGT | 8075 |
| Zbtb7a | GACTCCTCTTCGGCTCTGGC | 8076 |
| Zbtb7a | GTCGTGGGAGAGGTCTGGAG | 8077 |
| Zbtb7a | GATGCTCGCGACTCCCTTCC | 8078 |
| Zbtb7a | GGGAGTCGCGAGCATCATAC | 8079 |
| Zbtb7a | GCGAAAGAACTACAGAGCCC | 8080 |
| Zbtb7a | GGAGAGGTCTGGAGAGGGAG | 8081 |
| Zbtb7a | GGTTTGTCCGAGGGCAAGAG | 8082 |
| Zbtb7b | GACAAGAGCTGGCTGAGGAG | 8083 |
| Zbtb7b | GTGTATATGGGATTGATATG | 8084 |
| Zbtb7b | GTGAAGTCAAGGTAAGGGCA | 8085 |
| Zbtb7b | GGCTTAAGGACAGGGTCTTG | 8086 |
| Zbtb7b | GTGGCATTGGCAGGACTGGA | 8087 |
| Zbtb7b | GTCCTTATTGGGCGGAGGGA | 8088 |
| 2btb7b | GGAAAGTTCTGAGATGAACT | 8089 |
| Zbtb7b | GCAGACTGCTCAGGIGGAGG | 8090 |
| Zbtb7b | GACCCTGACAGTAGAAGGAA | 8091 |
| Zbtb7b | GGGCAGAGACCTTAGGAGGT | 8092 |
| Zc3h11a | GGGACCGGAATTTCTTTCTG | 8093 |
| Zc3h11a | GGCATAAGAGCTGGAATGAG | 8094 |
| Zc3h11a | GTCACGTGTCACGGAGGCAC | 8095 |
| Zc3h11a | GTGGCATAAGAGCTGGAATC | 8096 |
| Zc3h11a | GGAATTTCTTTCTGTGGACC | 8097 |
| Zc3h11a | GCATTATCCCTTAGATGCCA | 8098 |
| Zc3h11a | GGGTATGTTCCTTGTCCATA | 8099 |
| Zc3h11a | GGATGGAATTGAGGCATACA | 8100 |
| Zc3h11a | GGTCATAGGGTCACGTGTCA | 8101 |
| Zeb1 | GAAGGAACTAAGTTTCTTCT | 8102 |
| Zeb1 | GTGACAGGTGATCTAGGCGC | 8103 |
| Zeb1 | GCTCAGGTGTGGTGGAGTAG | 8104 |
| Zeb1 | GGAACCTTGTTGCTAGGGCC | 8105 |
| Zeb1 | GCCAGGTACTCAAGATGCCA | 8106 |
| Zeb1 | GAGTCTGCCATACCCAAGGA | 8107 |
| Zeb1 | GAGCAGTTGTCGCACTGGGA | 8108 |
| Zeb1 | GGAGTAGCGGAGAATAGTGC | 8109 |
| Zeb1 | GGAGAGCTTACGGTCTAGAA | 8110 |
| Zeb1 | GTAAGACTGGCTTACAAGTC | 8111 |
| 2eb2 | GAGATCAGTTCTAACCTGCT | 8112 |
| Zeb2 | GTATGAGGGAATGCACACGG | 8113 |
| Zeb2 | GTGCACACCATTCACAGAAC | 8114 |
| Zeb2 | GTAATCCAATCAGGTTACAT | 8115 |
| Zeb2 | GGCGGCAGAGAAAGGGTTAA | 8116 |
| Zeb2 | GTACTATGCTGGCCAATCTC | 8117 |
| Zeb2 | GGGTGACACTAAACTGTGTG | 8118 |
| Zeb2 | GTGGTACAGGGAAGATCGCG | 8119 |
| Zeb2 | GTCTCATTGTGCCTTTGCAC | 8120 |
| Zeb2 | GCAGGCACCCAGGTAGCTAC | 8121 |
| Zfhx3 | GGCAGCCTGAAGCAGGTCTA | 8122 |
| Zfhx3 | GGTAACAGACTGCGCCCAAC | 8123 |
| Zfhx3 | GAGACTGATGGATCAGGGTT | 8124 |
| Zfhx3 | GCCTGCACCAACCCAGGAAC | 8125 |
| Zfhx3 | GAAATGGTCTGTGGCTCCTA | 8126 |
| Zfhx3 | GCTGGCATGCTGACATCCTC | 8127 |
| Zfhx3 | GTGGATTTCGGAGAAATTGA | 8128 |
| Zfhx3 | GGGCACTTCTAGGTCTCCCA | 8129 |
| Zfhx3 | GGACTTTAGCCAATGTGGAC | 8130 |
| Zfp105 | GGAGAAAGCATTCAAGTGTG | 8131 |
| Zfp105 | GTCCACAGTCTTTGCGCTCA | 8132 |
| Zfp105 | GCCTGTGAGTGCAGTGAGTG | 8133 |
| Zfp105 | GTATTGTGAAACTCATTTGG | 8134 |
| Zfp105 | GTCGTACACCTCGGTGCCCA | 8135 |
| Zfp105 | GTTCAGTAAGGTTTCTGTGT | 8136 |
| Zfp105 | GAAATGATAAACCCAGAGGA | 8137 |
| Zfp105 | GAAGGCAGCGCCCTTTACTC | 8138 |
| Zfp148 | GCGTTGCATATTGAGGTTAG | 8139 |
| Zfp148 | GAGCTGGGAAAGCCACTGAG | 8140 |
| Zfp148 | GAGGGCGCTCCTAAGAAACC | 8141 |
| 2fp148 | GCCAGAATCAAAGCCACGCT | 8142 |
| Zfp148 | GACTCAAGAATTACAGTTTC | 8143 |
| Zfp148 | GCAAACCGCGATGCAGGATG | 8144 |
| Zfp148 | GACTTCGTTGGCCCAAACCA | 8145 |
| Zfp148 | GGTGCCCACATCCTGCATCG | 8146 |
| Zfp148 | GGAAGAAATTTAAGATGGAA | 8147 |
| Zfp2 | GGACTAGTTGGGAAGGATGG | 8148 |
| Zfp2 | GCACATGAATGAGGGTCGCT | 8149 |
| Zfp2 | GGGCATAGTGGACACCAAAG | 8150 |
| Zfp2 | GTACCTTGGTTCCCTATCCT | 8151 |
| Zfp2 | GCAACTGGAAGCCCTGTCGA | 8152 |
| 2fp2 | GAAGGACTAGTTGGGAAGGA | 8153 |
| Zfp2 | GAAGCCCTGTCGAGGGCTCA | 8154 |
| Zfp2 | GTTCCGAACCAAATGTCGGA | 8155 |
| Zfp2 | GGCTGTTAGGCCTCTGCCAT | 8156 |
| Zfp2 | GCCATTGCCTTCCTCCTTCC | 8157 |
| Zfp239 | GAAAGTGATACCATACATAT | 8158 |
| Zfp239 | GGCCAACCACAACCTCAAAC | 8159 |
| Zfp239 | GCCAACCACAACCTCAAACT | 8160 |
| Zfp239 | GGGCTTTCACAACGTTCCTG | 8161 |
| Zfp239 | GCTAAGTTTCACTTACCAAC | 8162 |
| Zfp239 | GAGCTCACTTCCGGCGACGA | 8163 |
| Zfp239 | GGACCATGTGGGCTACAGAT | 8164 |
| Zfp239 | GCAGAAACTAGTAACAGAAT | 8165 |
| Zfp239 | GCTACTTCCGAGCTCACTTC | 8166 |
| Zfp281 | GTGATTGGTGGCCCTGCTGA | 8167 |
| Zfp281 | GTTGTTCCTTATTCAGTTGG | 8168 |
| Zfp281 | GGCTAGCCAGGCGGAAACTG | 8169 |
| Zfp281 | GCGTGAATGAGAGAAATGAC | 8170 |
| Zfp281 | GCCCAAGCATGAAGGGCAGT | 8171 |
| Ztp281 | GTTACGAGTTTAAAGCTTTC | 8172 |
| Zfp281 | GCTCAAGGTACGACTTCTAA | 8173 |
| Zfp281 | GACAGTGGCTGAGGGTTTCT | 8174 |
| Zfp281 | GGTCCTGTGAACTAAATCTA | 8175 |
| Zfp35 | GACTCCCAGCTTTAGCATAG | 8176 |
| Zfp35 | GAAGCAGTGCCCAGACCACT | 8177 |
| Zfp35 | GCTAACCTGCCAAGTGGTCT | 8178 |
| Zfp35 | GGTCACGAAGAGCTAATAAC | 8179 |
| Zfp35 | GCCAGGTCTCATCACGGTCT | 8180 |
| Zfp35 | GACTCCGAGAGAAGGGTGCA | 8181 |
| Zfp35 | GGTAAATGAAGAAGCTCAGG | 8182 |
| Zfp35 | GCTGGTAAATGAAGAAGCTC | 8183 |
| Zfp35 | GCAGGGAAGAAGGGAAAGGG | 8184 |
| Zfp36 | GGATAGAAGACGGTTTAAGC | 8185 |
| Zfp36 | GGGCTACTCTCTAAAGGATG | 8186 |
| Zfp36 | GAGCCAAGGCACAAGGTGTG | 8187 |
| Zfp36 | GCTTCCTGGAAGCCGTGACG | 8188 |
| Zfp36 | GGTCTAAGACTTAAAGATCT | 8189 |
| Zfp36 | GGTTGTGTACGACCAACTGG | 8190 |
| Zfp36 | GTGCGTTGCGTATCGAGGTA | 8191 |
| Zfp36 | GGCAGTCGGGAAGAGAACTG | 8192 |
| Zfp36 | GACTCAGCAGATAGAGGAGG | 8193 |
| Zfp384 | GAAGGAAGGAGCCGAGGGAG | 8194 |
| Zfp384 | GCGGCAGCAGGAAAGGAAAG | 8195 |
| Zfp384 | GTGGGAGGATGGAAGCTAGA | 8196 |
| Zfp384 | GCTACCTTCACTAATCTGCT | 8197 |
| Zfp384 | GGCTTCCGGAAGAGACCTCT | 8198 |
| Zfp384 | GCTGCCTTCACGTCCGGTTT | 8199 |
| Zfp384 | GAGAGCCGCTCGAGCACATC | 8200 |
| Zfp384 | GTCACAGTCTTAAGAGAGGT | 8201 |
| Zfp384 | GTGAAGGCAGCGTGTGTTAA | 8202 |
| Zfp410 | GAACACTGATCAGTACTCTA | 8203 |
| Zfp410 | GTTGTGGGAAGGTACCATTG | 8204 |
| Zfp410 | GAGAAGATAAATGAGTGGTT | 8205 |
| Zfp410 | GGTAGCTTTCTCCGGAGCTG | 8206 |
| Zfp410 | GGTCCGCGAAATCTGAAACC | 8207 |
| Zfp410 | GCATAAGTACATAGGATTTC | 8208 |
| Zfp410 | GAAATGTTCATGGCCCAAAG | 8209 |
| Zfp410 | GTGGCAAGGAGATGTTTACT | 8210 |
| 2fp410 | GCATTCCAGTCCATGATGAC | 8211 |
| Zfp42 | GTTTCCTTTCACCTACAATT | 8212 |
| Zfp42 | GGAGTAACTAACTCCGAGCT | 8213 |
| Zfp42 | GAGTCTCAAGGCCAGGCGAT | 8214 |
| Zfp42 | GCATTCCAGCCTACCCAGCT | 8215 |
| Zfp42 | GCCTGGACAAGGATTCACGT | 8216 |
| Zfp42 | GTACAATCTTGTCCTAGGTA | 8217 |
| Zfp42 | GGAATGAGACCTACCAAAGG | 8218 |
| Zfp42 | GTGAATCCTTGTCCAGGCCC | 8219 |
| Zfp42 | GTAGCCTTCAGCAAATTTCG | 8220 |
| Zfp42 | GAATCCTTGTCCAGGCCCTG | 8221 |
| Zfp423 | GCTGGACATCTCTAGGCCGT | 8222 |
| Zfp423 | GGGAGGGAAATGGTCAGGGA | 8223 |
| Zfp423 | GGCTCGGATCAGACGGAGAG | 8224 |
| Zfp423 | GCTTGGGCTCTGACAGCACT | 8225 |
| Zfp423 | GCCTAGGGATGACTGATCCC | 8226 |
| Zfp423 | GTCCATGGAGGCAGCTAGCG | 8227 |
| Zfp423 | GGAAGGGAGGGAAATGGTCA | 8228 |
| Zfp423 | GCCTGCCTCTTTCCACCCTC | 8229 |
| Zfp423 | GCTGCCTCTCCTAGGTGGAG | 8230 |
| Zfp423 | GGCCTCAAAGGGCAGTTTAG | 8231 |
| Zfp628 | GTTCGCCAGATGCAGAATCC | 8232 |
| Zfp628 | GTCATGACGCACGAAGGCGG | 8233 |
| Zfp691 | GGCCTTGAAGGCTGATGTTT | 8234 |
| Zfp691 | GACTCTAAAGACACGGTGTG | 8235 |
| Zfp691 | GGCGGATCCTCTCTGGGTTC | 8236 |
| Zfp691 | GTCAAATCTAATAGTCTTGT | 8236 |
| Zfp691 | GTAGGACGGCCAATAAACAT | 8238 |
| Zfp691 | GGAATGACCTCCAGCCAGGT | 8239 |
| Zfp691 | GGTTTAGTGCGCGGCATGCT | 8240 |
| Zfp691 | GCAGGGAGGGTAGTAAGACT | 8241 |
| Zfp691 | GAATTCAATAATCTCTGACC | 8242 |
| Zfp740 | GTTTGGCTAGGTCAAGACTG | 8243 |
| Zfp740 | GAGGGCAGAACTGTGTTGGA | 8244 |
| Zfp740 | GTACTGAGTGTTCTTTGAGA | 8245 |
| Zfp740 | GACTATTCTAAGTGCACACT | 8246 |
| Zfp740 | GAGCTGGGATTTGCCATCTT | 8247 |
| Zfp740 | GGGCGCTAGTATTCGAGGAT | 8248 |
| Zfp740 | GAACTGTCCCGGCTTCTCTT | 8249 |
| Zfp740 | GGTTCGTAAATTCGCCGCCG | 8250 |
| Zfp740 | GGGCCGAAAGAGCAGCCAAC | 8251 |
| Zfp740 | GTGGGACTTGTTGGAATTCA | 8252 |
| Zfpm1 | GGCTTAGGGTCTGGGAGTCC | 8253 |
| Zfpm1 | GGTGGAAGATAGAGAAAGGA | 8254 |
| Zfpm1 | GTTCATGACCAGAGCCAGCA | 8255 |
| Zfpm1 | GGACCCTGTGTAGGTCTCAA | 8256 |
| Zfpm1 | GCTGGCATATGGTGAGAGGC | 8257 |
| Zfpm1 | GTCTATCTAGAAACGAGGGT | 8258 |
| Zfpm1 | GCTGAGGATTGGACAGACGT | 8259 |
| Zfpm1 | GCCTTTCAGGGAGCCAGCAG | 8260 |
| Zfpm1 | GCCACAGTGAACATCTGCCA | 8261 |
| Zfpm1 | GGGACAGGGTCGACGAAAGG | 8262 |
| Zfx | GCCGTAAATGTGTGTGTGTG | 8263 |
| Zfx | GTAATCTTCAGCACACTAAA | 8264 |
| Zfx | GGATAAAGCAAATTGCAAAC | 8265 |
| Zfx | GTGACGTGACGTGCTGACGG | 8266 |
| Zfx | GGGCGCTGTCACGGAAACTC | 8267 |
| Zfx | GCTCTGGAAACTGAAAGGAA | 8268 |
| Zfx | GGAAATTCGGGCTATGATAC | 8269 |
| Zhx1 | GAGGAGCCGAGAGTGACAGT | 8270 |
| Zhx1 | GAGCGCGGGACTGTTGACAG | 8271 |
| Zhx1 | GCTGACTTTGAAAGCTTGTT | 8272 |
| Zhx1 | GTGCAAGGATGCCAGATAAT | 8273 |
| Zhx1 | GCCGAGCAATCGCTTGAACG | 8274 |
| Zhx1 | GACTCTCCCTGACAGAGGGC | 8275 |
| Zhx1 | GCCCTTGTGGGTTCAGGGAG | 8276 |
| Zhx1 | GCACTCTGCATTTACATGAA | 8277 |
| Zhx1 | GGCTTCTAGTGACAGAGGCG | 8278 |
| Zhx1 | GAGGGCTGAAGGGCAGAGGT | 8279 |
| Zic1 | GGAAGGGTGGCTGTTGGGAG | 8280 |
| Zic1 | GTCTCCGAGAGCAGCAGTTG | 8281 |
| Zic1 | GTTAGGAAGTAGAAATTCTA | 8282 |
| Zic1 | GTTAAGCATCTATGCCTGTG | 8283 |
| Zic1 | GGGCAAAGACAGGTTAATCG | 8284 |
| Zic1 | GTCAAGCGCTTTACAATACC | 8285 |
| Zic1 | GGTGGGAGCAAGCCACACAA | 8286 |
| Zic1 | GCGAGTGTTGTATGTTTGCA | 8287 |
| Zic1 | GGGCAGAGTGAGCGAGTTGG | 8288 |
| Zic2 | GCCGCGTTGACTTCATTCGG | 8289 |
| Zic2 | GCACACAAGCTGGCAGGGAG | 8290 |
| Zic2 | GAACCAATGTGGCTGTGGAC | 8291 |
| Zic2 | GTTTATTTCGGTGGGCGCTG | 8292 |
| Zic2 | GAACTCTTGAATTAGCCGGC | 8293 |
| Zic2 | GGGAGCCTTGGTAGAAAGGT | 8294 |
| Zic2 | GGCCGAGCCTTGGATAAGGA | 8295 |
| Zic2 | GGGTTGTGCAGCTGCAGCAG | 8296 |
| Zic2 | GCCCAGTCTATGGGTGCGTT | 8297 |
| Zic2 | GCTTGGTGACTCTATAGCTA | 8298 |
| Zic3 | GCTTGCTGCCGGCTTAGAGC | 8299 |
| Zic3 | GGCAAAGCACTAGCAAAGGG | 8300 |
| Zic3 | GCAAAGCCCGGGATGTTAAG | 8301 |
| Zic3 | GTGGACAGCTACCTCTAGTT | 8302 |
| Zic3 | GCAGCGCGAAGCGGAGAACA | 8303 |
| Zic3 | GAGAAGTGGGAAGGTGGGAA | 8304 |
| Zic3 | GCCCTGACTCTTAGTCGCGA | 8305 |
| Zic3 | GGTGGGAGTTTCCATATTTG | 8306 |
| Zic3 | GAGTCTCGAACGCAGGGCAA | 8307 |
| Zic3 | GCAACGACAAGTATCTTCTT | 8308 |
| Zscan26 | GTAAAGCCAGAGGAGGAAAC | 8309 |
| Zscan26 | GGGAGGACCAGGACTCAACT | 8310 |
| Zscan26 | GCAGGGCCCAAATCTGTCAG | 8311 |
| Zscan26 | GTAGGATGTCAAAGACTTGC | 8312 |
| Zscan26 | GTGACTAACAGTTAGACAAA | 8313 |
| Zscan26 | GCTTGAGAAGGTAAAGCCAG | 8314 |
| Zscan26 | GATTGGATGGCCTGAGGATG | 8315 |
| Zscan26 | GGAGCCGGAAACTACTGGGC | 8316 |
| Zscan26 | GAGACTTTAAACCATTTACC | 8317 |

All publications and patents mentioned in the above specification are herein incorporated by reference as if expressly set forth herein. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in relevant fields are intended to be within the scope of the following claims.

1-20. (canceled)

21. A method for converting cell fate of a population of cells, the method comprising:
regulating an expression level of a first endogenous gene and an expression level of a second endogenous gene in the population of cells, via using (i) a CRISPR-Cas protein, (ii) a first guide ribonucleic acid molecule (first guide RNA) exhibiting specific binding to the first endogenous gene, and (iii) a second guide ribonucleic acid molecule (second guide RNA) exhibiting specific binding to the second endogenous gene, wherein the first endogenous gene and the second endogenous gene are different,
wherein the regulating synergistically yields a greater degree of conversion of the cell fate of the population of cells, as compared to that from regulating an expression level of only one of the first endogenous gene and the second endogenous gene.

22. The method of claim 21, wherein the regulating induces expression of a cellular marker indicative of the conversion in the population of cells.

23. The method of claim 22, wherein, upon the regulating, at least 50% of the population is positive for the cellular marker.

24. The method of claim 22, wherein a portion of the population of cells positive for the cellular marker is greater than a sum of (i) a portion of a first control population of cells subjected to regulation of the expression of the first endogenous gene but not that of the second endogenous gene and (ii) a portion of a second control population of cells subjected to regulation of the expression of the second endogenous gene but not that of the first endogenous gene.

25. The method of claim 21, wherein the population of cells is contacted by the first guide RNA and the second guide RNA substantially simultaneously.

26. The method of claim 21, wherein the CRISPR-Cas protein is a nuclease dead CRISPR-Cas protein.

27. The method of claim 21, wherein the CRISPR-Cas protein is CRISPR-Cas9.

28. The method of claim 21, wherein the CRISPR-Cas protein is fused to a gene activator.

29. The method of claim 21, wherein the CRISPR-Cas protein is fused to a gene repressor.

30. The method of claim 21, wherein the first endogenous gene or the second endogenous gene is a non-coding gene.

31. The method of claim 21, wherein the first endogenous gene or the second endogenous gene is a transcription factor.

32. The method of claim 21, wherein the first endogenous gene or the second endogenous gene is a cell differentiation factor.

33. The method of claim 21, wherein at least one of the first guide RNA and the second guide RNA is substantially free of a RNA homopolymer sequence having a length greater than 3 nucleotides.

34. The method of claim 21, wherein at least one of the first guide RNA and the second guide RNA exhibits less than 2 mismatches with a different genomic sequence of the population of cells.

35. The method of claim 21, wherein at least one of the first guide RNA and the second guide RNA comprises a GC content of at least about 30%.

36. The method of claim 21, wherein at least one of the first guide RNA and the second guide RNA comprises a GC content of at most about 70%.

37. The method of claim 21, wherein at least one of the first guide RNA and the second guide RNA comprises a GC content of between about 30% and about 70%.

38. The method of claim 21, wherein the population of cells comprises mammalian cells.

39. The method of claim 21, wherein the population of cells comprises stem cells.

40. The method of claim 21, wherein the conversion comprises a change of cell type in the population of cells.
